(19) United States
(12) Patent Application Publication
(10) Pub. No.: US 2024/0425860 A1
(43) Pub. Date: Dec. 26, 2024

(54) OLIGONUCLEOTIDES FOR SARS-CoV-2 MODULATION

(71) Applicant: UNIVERSITY OF MASSACHUSETTS, Boston, MA (US)

(72) Inventors: Anastasia Khvorova, Westborough, MA (US); Jonathan Watts, Worcester, MA (US); Zachary Kennedy, Worcester, MA (US); Annabelle Biscans, Gothenburg (SE); Bruno Miguel da Cruz Godinho, Worcester, MA (US); Chantal Ferguson, Worcester, MA (US); Dimas Echeverria Moreno, Worcester, MA (US); Ken Yamada, Westborough, MA (US); Daniel O'Reilly, Barnham (GB); Kathryn Monopoli, Boston, MA (US); Vignesh Narayan Hariharan, Southborough, MA (US); Qi Tang, Worcester, MA (US); Sarah Davis, Wellesley, MA (US); Samuel Hildebrand, Boston, MA (US); Socheata Ly, Boston, MA (US); Minwook Shin, Worcester, MA (US); Pranathi Meda Krishnamurthy, Boston, MA (US); Nicholas McHugh, Boston, MA (US); Jacquelyn Sousa, Boston, MA (US); Jillian Caiazzi, Durham, NC (US); Yann Thillier, Boston, MA (US); Gitali Devi, Boston, MA (US)

(21) Appl. No.: 18/675,670

(22) Filed: May 28, 2024

Related U.S. Application Data
(62) Division of application No. 17/333,839, filed on May 28, 2021, now Pat. No. 12,077,758.
(60) Provisional application No. 63/084,817, filed on Sep. 29, 2020.
(60) Provisional application No. 63/031,222, filed on May 28, 2020.

(51) Int. Cl. C12N 15/113

(52) U.S. Cl. CPC C12N 15/1131; C12N 15/1136; C12N 15/1137; C12N 2310/14; C12N 2310/315; C12N 2310/321; C12N 2310/351; C12N 2310/3515; C12N 2320/32

(57) ABSTRACT

This disclosure relates to novel SARS-CoV-2 targeting sequences. Novel SARS-CoV-2 targeting oligonucleotides for the treatment of SARS-CoV-2 infection are also provided.

### CROSS-REFERENCE TO RELATED APPLICATIONS

This application is a divisional of U.S. patent application Ser. No. 17/333,839, filed May 18, 2021, which claims the benefit of U.S. Provisional Application Ser. No. 63/031,222, filed May 28, 2020, and U.S. Provisional Application Ser. No. 63/084,817, filed Sep. 29, 2020, the entire disclosures of which are incorporated herein by reference.

### SEQUENCE LISTING

The instant application contains a Sequence Listing which has been submitted electronically in XML format and is hereby incorporated by reference in its entirety. Said XML file, created on May 27, 2024, is named 754968 SA9-258 DIV.xml and is 2,661,424 bytes in size.

### FIELD OF THE INVENTION

This disclosure relates to novel SARS-CoV-2 targeting sequences, novel branched oligonucleotides, and novel methods for treating and preventing SARS-CoV-2-related infection.

### BACKGROUND

SARS-CoV-2 (severe acute respiratory syndrome coronavirus 2) is a highly infectious virus that causes severe respiratory illness. The SARS-CoV-2 genome encodes for four structural proteins, S (spike), E (envelope), M (membrane) and N (nucleocapsid). The spike protein plays a critical role in viral entry into a host cell. SARS-CoV-2 is the causative agent of the COVID-19 epidemic that has infected and kills millions of people worldwide.

RNAi-based therapeutics are revolutionizing human medicine. Currently, a single subcutaneous injection of chemically modified oligonucleotide compounds supports up to 12-months of target silencing in the liver with a clean adverse events profile. The ability to develop RNAi-based drugs is dependent on efficient delivery to the targeted tissues. Currently, the liver is the only tissue validated for clinical delivery.

With the current clinical approaches, it is not possible to halt or cure SARS-CoV-2 infection. The highly infectious virus is spreading throughout the world, leaving a path of destruction and death. Survivors of a severe infection with SARS-CoV-2 often present with long lasting lung injury and scarring.

There is a clear need for a therapeutic that can effectively neutralize SARS-CoV-2 particles from causing infection, and especially to selectively do so in the lung. This could be accomplished using optimized RNAi-based therapeutics, which is addressed in the present application.

### SUMMARY

In one aspect, the disclosure provides an RNA molecule having a length of from about 8 nucleotides to about 80 nucleotides; and a nucleic acid sequence that is substantially complementary to a SARS-CoV-2 nucleic acid sequence of SEQ ID NO: 1. In certain embodiments, the RNA molecule is from 8 nucleotides to 80 nucleotides in length (e.g., 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, 60 nucleotides, 61 nucleotides, 62 nucleotides, 63 nucleotides, 64 nucleotides, 65 nucleotides, 66 nucleotides, 67 nucleotides, 68 nucleotides, 69 nucleotides, 70 nucleotides, 71 nucleotides, 72 nucleotides, 73 nucleotides, 74 nucleotides, 75 nucleotides, 76 nucleotides, 77 nucleotides, 78 nucleotides, 79 nucleotides, or 80 nucleotides in length).

In certain embodiments, the RNA molecule is from 10 to 50 nucleotides in length (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides in length).

In certain embodiments, the RNA molecule comprises about 15 nucleotides to about 25 nucleotides in length. In certain embodiments, the RNA molecule is from 15 to 25 nucleotides in length (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length).

In one aspect, the disclosure provides an oligonucleotide compound comprising 15 to 35 bases in length, comprising a sequence substantially complementary to a SARS-CoV-2 nucleic acid sequence of SEQ ID NO: 1.

In certain embodiments, the oligonucleotide compound comprises a sequence substantially complementary to a SARS-CoV-2 nucleic acid sequence of any one of SEQ ID NOs: 2-10.

In certain embodiments, the oligonucleotide compound comprises a sequence substantially complementary to a SARS-CoV-2 nucleic acid sequence of any one of the 45-nucleotide target gene regions in Table 6A.

In certain embodiments, the oligonucleotide compound comprises a sequence substantially complementary to a SARS-CoV-2 nucleic acid sequence of any one of the 20-nucleotide targets in Table 6A.

In certain embodiments, the oligonucleotide compound comprises a sequence substantially complementary to a SARS-CoV-2 nucleic acid sequence of any one of the 45-nucleotide target gene regions of 7a_27751, N_29293, Orf1a_2290, and Orf1ab_18571 in Table 6A.

In certain embodiments, the oligonucleotide compound comprises a sequence substantially complementary to a SARS-CoV-2 nucleic acid sequence of any one of the 20-nucleotide targets of 7a_27751, N_29293, Orf1a_2290, and Orf1ab_18571 in Table 6A.

In certain embodiments, the oligonucleotide compound comprises complementarity to at least 10, 11, 12 or 13 contiguous nucleotides of the SARS-CoV-2 nucleic acid sequence of any one of SEQ ID NOs: 1-10. In certain embodiments, the oligonucleotide compound comprises no more than 3 mismatches with the SARS-CoV-2 nucleic acid sequence of any one of SEQ ID NOs: 1-10. In certain embodiments, the oligonucleotide compound comprises full complementarity to the SARS-CoV-2 nucleic acid sequence of any one of SEQ ID NOs: 1-10.

In another aspect, the disclosure provides an oligonucleotide compound comprising 15 to 35 bases in length, comprising a sequence substantially complementary to an Angiotensin I Converting Enzyme 2 (ACE2) nucleic acid sequence of any one of the 45-nucleotide target gene regions in Table 11D or Table 12C.

In certain embodiments, the oligonucleotide compound comprises a sequence substantially complementary to an ACE2 nucleic acid sequence of any one of the 45-nucleotide target gene regions of ACE2_119, ACE2_336, ACE2_349, ACE_1034, ACE_1775, ACE_784, ACE_908, and ACE_1071, as recited in Table 12C

In another aspect, the disclosure provides an oligonucleotide compound comprising 15 to 35 bases in length, comprising a sequence substantially complementary to a FURIN nucleic acid sequence of any one of the 45-nucleotide target gene regions in Table 11C or Table 12D.

In certain embodiments, the oligonucleotide compound comprises a sequence substantially complementary to a FURIN nucleic acid sequence of any one of the 45-nucleotide target gene regions of FURIN_443, FURIN_1959, FURIN_2711, FURIN_2712, FURIN_3524, and FURIN_3526, as recited in Table 12D.

In one aspect, the disclosure provides an oligonucleotide compound comprising 15 to 35 bases in length, comprising a sequence substantially complementary to an Interleukin 6 (IL-6) nucleic acid sequence of any one of the 45-nucleotide target gene regions in Table 11B or Table 12B.

In one aspect, the disclosure provides an oligonucleotide compound comprising 15 to 35 bases in length, comprising a sequence substantially complementary to an Interleukin 6 Receptor (IL-6R) nucleic acid sequence of any one of the 45-nucleotide target gene regions in Table 12E.

In one aspect, the disclosure provides an oligonucleotide compound comprising 15 to 35 bases in length, comprising a sequence substantially complementary to a Transmembrane Serine Protease 2 (TMPRSS2) nucleic acid sequence of any one of the 45-nucleotide target gene regions in Table 11A or Table 12A.

In certain embodiments, the oligonucleotide compound comprises one or more naturally occurring nucleotides.

In certain embodiments, the oligonucleotide compound comprises one or more modified nucleotide.

In certain embodiments, the one or more modified nucleotides each independently comprise a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 2′-O-alkyl, 2′-O-alkoxy, 2′-O-alkylamino, 2′-NH₂, and a constrained nucleotide.

In certain embodiments, the constrained nucleotide is selected from the group consisting of a locked nucleic acid (LNA), an ethyl-constrained nucleotide, a 2′-(S)-constrained ethyl (S-cEt) nucleotide, a constrained MOE, a 2′-0,4′-C-aminomethylene bridged nucleic acid (2′,4′-BNA^{NC}), an alpha-L-locked nucleic acid, a tricyclo-DNA, and any combination thereof.

In certain embodiments, the constrained nucleotide is a locked nucleic acid (LNA), a 2′-(S)-constrained ethyl (S-cEt) nucleotide, and a combination thereof

In certain embodiments, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, each modification of the phosphate group is independently selected from the group consisting of a phosphorothioate, phosphonoacetate (PACE), thiophosphonoacetate (thioPACE), amide, triazole, phosphonate, and phosphotriester modification.

In certain embodiments, the modification of the phosphate group is phosphorothioate

In certain embodiments, the oligonucleotide compound comprises 4-16 phosphorothioate modifications. In certain embodiments, the oligonucleotide compound comprises 6-13 phosphorothioate modifications.

In certain embodiments, the oligonucleotide compound comprises at least one modified internucleotide linkage.

In certain embodiments, the oligonucleotide compound comprises at least one modified internucleotide linkage of Formula I:

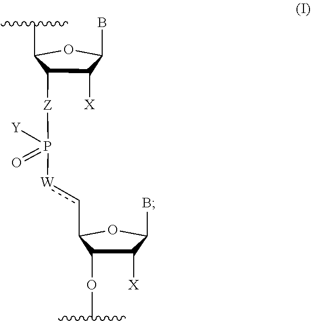

wherein:
B is a base pairing moiety;
W is selected from the group consisting of O, OCH₂, OCH, CH₂, and CH;
X is selected from the group consisting of halo, hydroxy, and C₁₋₆ alkoxy;
Y is selected from the group consisting of O⁻, OH, OR, NH⁻, NH₂, S⁻, and SH;
Z is selected from the group consisting of O and CH₂;
R is a protecting group; and
[symbol] is an optional double bond.

In certain embodiments, the oligonucleotide compound comprises at least 80% chemically modified nucleotides. In certain embodiments, the oligonucleotide compound is fully chemically modified.

In certain embodiments, the oligonucleotide compound comprises an antisense oligonucleotide or a double stranded (ds) RNA.

In certain embodiments, the dsRNA comprises an antisense strand and a sense strand. In certain embodiments, the antisense strand comprises about 15 nucleotides to 25 nucleotides in length. In certain embodiments, the sense strand comprises about 15 nucleotides to 25 nucleotides in length. In certain embodiments, the antisense strand is 20 nucleotides in length, 21 nucleotides in length, or 22 nucleotides in length. In certain embodiments, the sense strand is 15 nucleotides in length, 16 nucleotides in length, 18 nucleotides in length, or 20 nucleotides in length.

In certain embodiments, the dsRNA comprises a double-stranded region of 15 base pairs to 20 base pairs. In certain embodiments, the dsRNA comprises a double-stranded region of 15 base pairs, 16 base pairs, 18 base pairs, or 20 base pairs.

In certain embodiments, the dsRNA comprises a blunt-end. In certain embodiments, the dsRNA comprises at least one single stranded nucleotide overhang. In certain embodiments, the dsRNA comprises about a 2-nucleotide to 5-nucleotide single stranded nucleotide overhang. In certain embodiments, the dsRNA comprises a 2-nucleotide single stranded nucleotide overhang or a 5-nucleotide single stranded nucleotide overhang.

In certain embodiments, the dsRNA comprises at least 70% 2′-O-methyl nucleotide modifications.

In certain embodiments, the antisense strand comprises at least 70% 2′-O-methyl nucleotide modifications. In certain embodiments, the antisense strand comprises about 70% to 90% 2′-O-methyl nucleotide modifications. In certain embodiments, the sense strand comprises at least 65% 2′-O-methyl nucleotide modifications. In certain embodiments, the sense strand comprises 100% 2′-O-methyl nucleotide modifications.

In certain embodiments, the sense strand comprises one or more nucleotide mismatches between the antisense strand and the sense strand. In certain embodiments, the one or more nucleotide mismatches are present at positions 2, 6, and 12 from the 5′ end of sense strand. In certain embodiments, the nucleotide mismatches are present at positions 2, 6, and 12 from the 5′ end of the sense strand.

In certain embodiments, the antisense strand comprises a 5′ phosphate, a 5′-alkyl phosphonate, a 5′ alkylene phosphonate, or a 5′ alkenyl phosphonate. In certain embodiments, the antisense strand comprises a 5′ vinyl phosphonate.

In certain embodiments, the dsRNA comprises an antisense strand and a sense strand, each strand with a 5′ end and a 3′ end, wherein: (1) the antisense strand comprises a sequence substantially complementary to a SARS-CoV-2 nucleic acid sequence of any one of the 45-nucleotide target gene regions in Table 6A; (2) the antisense strand comprises alternating 2′-methoxy-ribonucleotides and 2′-fluoro-ribonucleotides; (3) the nucleotides at positions 2 and 14 from the 5′ end of the antisense strand are not 2′-methoxy-ribonucleotides; (4) the nucleotides at positions 1-2 to 1-7 from the 3′ end of the antisense strand are connected to each other via phosphorothioate internucleotide linkages; (5) a portion of the antisense strand is complementary to a portion of the sense strand; (6) the sense strand comprises alternating 2′-methoxy-ribonucleotides and 2′-fluoro-ribonucleotides; and (7) the nucleotides at positions 1-2 from the 5′ end of the sense strand are connected to each other via phosphorothioate internucleotide linkages.

In certain embodiments, the dsRNA comprises an antisense strand and a sense strand, each strand with a 5′ end and a 3′ end, wherein: (1) the antisense strand comprises a sequence substantially complementary to a SARS-CoV-2 nucleic acid sequence of any one of the 45-nucleotide target gene regions in Table 6A; (2) the antisense strand comprises at least 70% 2′-O-methyl modifications; (3) the nucleotides at positions 2, 6, 14, and 16 from the 5′ end of the antisense strand are not a 2′-methoxy-ribonucleotide; (4) the nucleotides at positions 1-2 to 1-7 from the 3′ end of the antisense strand are connected to each other via phosphorothioate internucleotide linkages; (5) a portion of the antisense strand is complementary to a portion of the sense strand; (6) the sense strand comprises at least 70% 2′-O-methyl modifications; (7) the nucleotides at positions 6, 7, 8 and 10 from the 5′ end of the sense strand are not a 2′-methoxy-ribonucleotide; and (8) the nucleotides at positions 1-2 from the 5′ end of the sense strand are connected to each other via phosphorothioate internucleotide linkages.

In certain embodiments, the dsRNA further comprises 1 to 5 internucleotide linkages of Formula I. In certain embodiments, the 3′ end of the antisense strand comprises 1 to 5 internucleotide linkages of Formula I. In certain embodiments, the 3′ end of the antisense strand comprises 4 consecutive internucleotide linkages of Formula I.

In certain embodiments, a functional moiety is linked to one or both of the 5′ end and 3′ end of the antisense strand. In certain embodiments, a functional moiety is linked to one or both of the 5′ end and 3′ end of the sense strand. In certain embodiments, a functional moiety is linked to the 3′ end of the sense strand.

In certain embodiments, the functional moiety comprises a hydrophobic moiety. In certain embodiments, the hydrophobic moiety is selected from the group consisting of fatty acids, steroids, secosteroids, lipids, gangliosides, nucleoside analogs, endocannabinoids, vitamins, and a mixture thereof. In certain embodiments, the steroid selected from the group consisting of cholesterol and Lithocholic acid (LCA). In certain embodiments, the fatty acid selected from the group consisting of Eicosapentaenoic acid (EPA), Docosahexaenoic acid (DHA) and Docosanoic acid (DCA). In certain embodiments, the vitamin is selected from the group consisting of choline, vitamin A, vitamin E, and derivatives or metabolites thereof. In certain embodiments, the vitamin is selected from the group consisting of retinoic acid and alpha-tocopheryl succinate.

In certain embodiments, the functional moiety is linked to one or both of the antisense strand and sense strand by a linker. In certain embodiments, the linker comprises a divalent or trivalent linker. In certain embodiments, the divalent or trivalent linker is selected from the group consisting of:

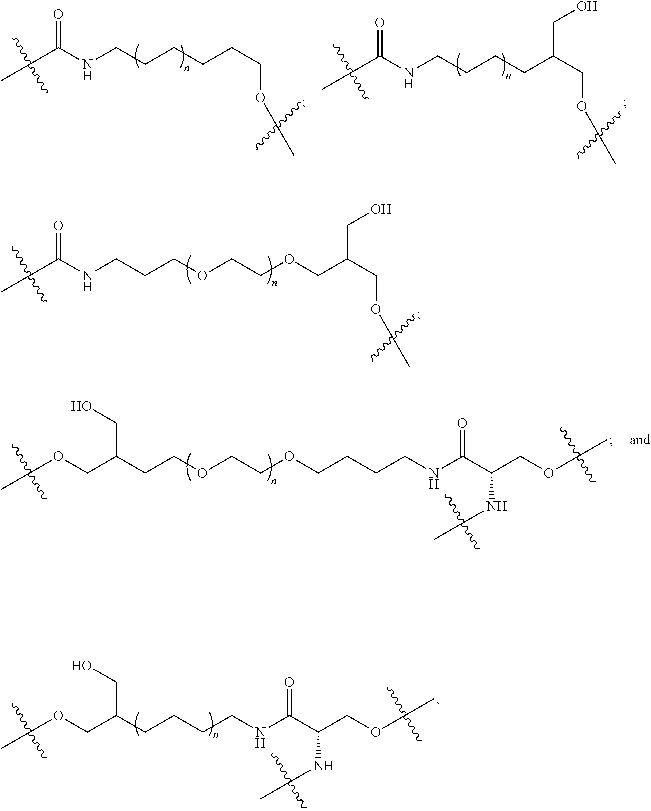

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, the linker comprises a dTdT dinucleotide.

In certain embodiments, the functional moiety is linked to the 3′ end of the sense strand by a dTdT dinucleotide followed by the linker

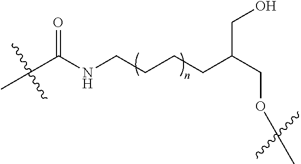

wherein n is 1.

In certain embodiments, the trivalent linker further links a phosphodiester or phosphodiester derivative.

In certain embodiments, the phosphodiester or phosphodiester derivative is selected from the group consisting of:

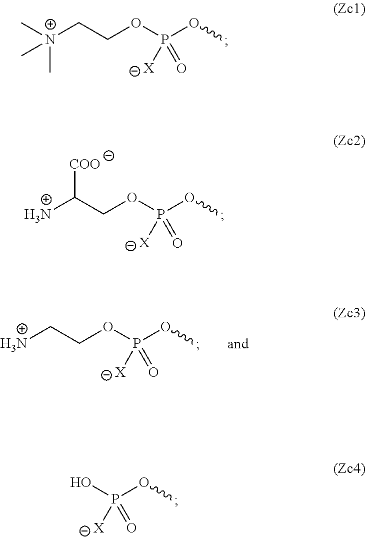

wherein X is O, S or BH₃.

In certain embodiments, the nucleotides at positions 1 and 2 from the 5′ end of antisense strand are connected to adjacent ribonucleotides via phosphorothioate linkages.

In one aspect, the disclosure provides a double stranded (ds) RNA, comprising an antisense strand and a sense strand, each strand with a 5′ end and a 3′ end, wherein: (1) the antisense strand comprises a sequence substantially complementary to a SARS-CoV-2 nucleic acid sequence of any one of the 45-nucleotide target gene regions in Table 6A; (2) the antisense strand comprises at least 70% 2′-O-methyl modifications; (3) the nucleotides at positions 2, 6, 14, and 16 from the 5′ end of the antisense strand are not a 2′-methoxy-ribonucleotide; (4) the nucleotides at positions 1-2 to 1-7 from the 3′ end of the antisense strand are connected to each other via phosphorothioate internucleotide linkages; (5) a portion of the antisense strand is complementary to a portion of the sense strand; (6) the sense strand comprises at least 70% 2′-O-methyl modifications; (7) the nucleotides at positions 6, 7, 8 and 10 from the 5′ end of the sense strand are not a 2′-methoxy-ribonucleotide; (8) the nucleotides at positions 1-2 from the 5′ end of the sense strand are connected to each other via phosphorothioate internucleotide linkages; and (9) the antisense strand comprises at least one modified intersubunit linkages of Formula II:

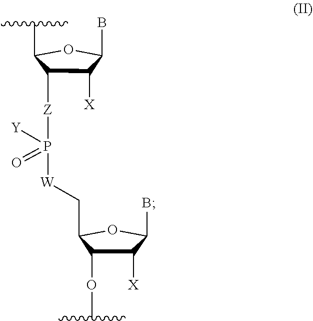

wherein:
B is a base pairing moiety;
W is O or O(CH₂)ₙ¹, wherein n¹ is 1 to 10;
X is selected from the group consisting of H, OH, OR¹, F, SH, SR, NR²₂ and C₁₋₆-alkoxy;
Y is selected from the group consisting of O⁻, OH, OR, OR₂, NH⁻, NH₂, NR²₂, BH₃, S⁻, R¹, and SH;
Z is O or O(CH₂)ₙ² wherein n2 is 1 to 10;
R¹ is alkyl, allyl or aryl; and
R² is alkyl, allyl or aryl.

In certain embodiments, the 3′ end of the antisense strand comprises four consecutive modified intersubunit linkages of Formula II.

In certain embodiments, the antisense strand comprises a 5′ vinyl phosphonate.

In certain embodiments, the antisense strand is 20 nucleotides in length, 21 nucleotides in length, or 22 nucleotides in length. In certain embodiments, the sense strand is 15 nucleotides in length, 16 nucleotides in length, 18 nucleotides in length, or 20 nucleotides in length.

In certain embodiments, a functional moiety is linked to the 3′ end of the sense strand.

In certain embodiments, the functional moiety comprises Eicosapentaenoic acid (EPA) or Docosanoic acid (DCA).

In certain embodiments, a functional moiety is linked to the 3′ end of the sense strand by a linker.

In certain embodiments, the linker comprises a dTdT dinucleotide.

In certain embodiments, the functional moiety is linked to the 3′ end of the sense strand by a dTdT dinucleotide followed by the linker

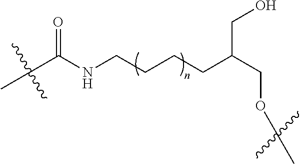

wherein n is 1.

In one aspect, the disclosure provides a combination comprising two or more oligonucleotide compounds or dsRNA recited above, wherein each oligonucleotide compound or dsRNA in the combination comprises complementarity to a different SARS-CoV-2 nucleic acid sequence.

In certain embodiments, the combination comprises two, three, four, or five oligonucleotide compounds or dsRNA.

In one aspect, the disclosure provides a combination comprising two or more oligonucleotide compounds for inhibiting the expression of a SARS-CoV-2 gene in a cell of an organism, wherein each oligonucleotide compound in the combination comprises complementarity to a different SARS-CoV-2 nucleic acid sequence.

In certain embodiments, the combination comprises a first oligonucleotide compound, a second oligonucleotide compound, and a third oligonucleotide compound, wherein:
i) the first oligonucleotide compound comprises a sequence substantially complementary to the 45-nucleotide target gene region orf1a_416, as recited in Table 6A;
ii) the second oligonucleotide compound comprises a sequence substantially complementary to the 45-nucleotide target gene region orf1a_9679, as recited in Table 6A; and
iii) the third oligonucleotide compound comprises a sequence substantially complementary to the 45-nucleotide target gene region orf1ab_21391, as recited in Table 6A; or
i) the first oligonucleotide compound comprises a sequence substantially complementary to the 45-nucleotide target gene region orf1a_416, as recited in Table 6A;
ii) the second oligonucleotide compound comprises a sequence substantially complementary to the 45-nucleotide target gene region orf1a_8744, as recited in Table 6A; and
iii) the third oligonucleotide compound comprises a sequence substantially complementary to the 45-nucleotide target gene region orf1ab_21391, as recited in Table 6A; or
i) the first oligonucleotide compound comprises a sequence substantially complementary to the 45-nucleotide target gene region orf1a_9679, as recited in Table 6A;
ii) the second oligonucleotide compound comprises a sequence substantially complementary to the 45-nucleotide target gene region orf1a_8744, as recited in Table 6A; and
iii) the third oligonucleotide compound comprises a sequence substantially complementary to the 45-nucleotide target gene region orf1ab_21391, as recited in Table 6A; or
i) the first oligonucleotide compound comprises a sequence substantially complementary to the 45-nucleotide target gene region orf1a_416, as recited in Table 6A;
ii) the second oligonucleotide compound comprises a sequence substantially complementary to the 45-nucleotide target gene region orf1a_9679, as recited in Table 6A; and
iii) the third oligonucleotide compound comprises a sequence substantially complementary to the 45-nucleotide target gene region 7a_27565, as recited in Table 6A; or
i) the first oligonucleotide compound comprises a sequence substantially complementary to the 45-nucleotide target gene region orf1a_416, as recited in Table 6A;
ii) the second oligonucleotide compound comprises a sequence substantially complementary to the 45-nucleotide target gene region orf1ab_21391, as recited in Table 6A; and
iii) the third oligonucleotide compound comprises a sequence substantially complementary to the 45-nucleotide target gene region 7a_27565, as recited in Table 6A; or
i) the first oligonucleotide compound comprises a sequence substantially complementary to the 45-nucleotide target gene region orf1ab_21391, as recited in Table 6A;
ii) the second oligonucleotide compound comprises a sequence substantially complementary to the 45-nucleotide target gene region 7a_27656, as recited in Table 6A; and
iii) the third oligonucleotide compound comprises a sequence substantially complementary to the 45-nucleotide target gene region 7a_27751, as recited in Table 6A; or
i) the first oligonucleotide compound comprises a sequence substantially complementary to the 45-nucleotide target gene region orf1a_416, as recited in Table 6A;
ii) the second oligonucleotide compound comprises a sequence substantially complementary to the 45-nucleotide target gene region S_23174, as recited in Table 6A; and
iii) the third oligonucleotide compound comprises a sequence substantially complementary to the 45-nucleotide target gene region E_26305, as recited in Table 6A; or
i) the first oligonucleotide compound comprises a sequence substantially complementary to the 45-nucleotide target gene region orf1a_9679, as recited in Table 6A;
ii) the second oligonucleotide compound comprises a sequence substantially complementary to the 45-nucleotide target gene region S_23174, as recited in Table 6A; and
iii) the third oligonucleotide compound comprises a sequence substantially complementary to the 45-nucleotide target gene region N_29293, as recited in Table 6A; or
i) the first oligonucleotide compound comprises a sequence substantially complementary to the 45-nucleotide target gene region orf1ab_21391, as recited in Table 6A;
ii) the second oligonucleotide compound comprises a sequence substantially complementary to the 45-nucleotide target gene region E_26305, as recited in Table 6A; and
iii) the third oligonucleotide compound comprises a sequence substantially complementary to the 45-nucleotide target gene region N_29293, as recited in Table 6A; or
i) the first oligonucleotide compound comprises a sequence substantially complementary to the 45-nucleotide target gene region S_23174, as recited in Table 6A;
ii) the second oligonucleotide compound comprises a sequence substantially complementary to the 45-nucleotide target gene region E_26470, as recited in Table 6A; and
iii) the third oligonucleotide compound comprises a sequence substantially complementary to the 45-nucleotide target gene region 7a_27565, as recited in Table 6A; or
i) the first oligonucleotide compound comprises a sequence substantially complementary to the 45-nucleotide target gene region S_23174, as recited in Table 6A;
ii) the second oligonucleotide compound comprises a sequence substantially complementary to the 45-nucleotide target gene region M_27123, as recited in Table 6A; and
iii) the third oligonucleotide compound comprises a sequence substantially complementary to the 45-nucleotide target gene region 7a_27656, as recited in Table 6A; or
i) the first oligonucleotide compound comprises a sequence substantially complementary to the 45-nucleotide target gene region S_23174, as recited in Table 6A;
ii) the second oligonucleotide compound comprises a sequence substantially complementary to the 45-nucleotide target gene region M_27032, as recited in Table 6A; and
iii) the third oligonucleotide compound comprises a sequence substantially complementary to the 45-nucleotide target gene region 7a_27751, as recited in Table 6A; or
i) the first oligonucleotide compound comprises a sequence substantially complementary to the 45-nucleotide target gene region orf1a_416, as recited in Table 6A;
ii) the second oligonucleotide compound comprises a sequence substantially complementary to the 45-nucleotide target gene region E_26305, as recited in Table 6A; and
iii) the third oligonucleotide compound comprises a sequence substantially complementary to the 45-nucleotide target gene region 7a_27565, as recited in Table 6A; or
i) the first oligonucleotide compound comprises a sequence substantially complementary to the 45-nucleotide target gene region orf1a_9679, as recited in Table 6A;
ii) the second oligonucleotide compound comprises a sequence substantially complementary to the 45-nucleotide target gene region E_26369, as recited in Table 6A; and
iii) the third oligonucleotide compound comprises a sequence substantially complementary to the 45-nucleotide target gene region 7a_27656, as recited in Table 6A; or
i) the first oligonucleotide compound comprises a sequence substantially complementary to the 45-nucleotide target gene region orf1ab_21391, as recited in Table 6A;
ii) the second oligonucleotide compound comprises a sequence substantially complementary to the 45-nucleotide target gene region M_27032, as recited in Table 6A; and
iii) the third oligonucleotide compound comprises a sequence substantially complementary to the 45-nucleotide target gene region 7a_27751, as recited in Table 6A; or
i) the first oligonucleotide compound comprises a sequence substantially complementary to the 45-nucleotide target gene region 3a_25868, as recited in Table 6A;
ii) the second oligonucleotide compound comprises a sequence substantially complementary to the 45-nucleotide target gene region 7a_27751, as recited in Table 6A; and
iii) the third oligonucleotide compound comprises a sequence substantially complementary to the 45-nucleotide target gene region S_23774, as recited in Table 6A.

In one aspect, the disclosure provides a combination comprising one or more oligonucleotide compounds for inhibiting the expression of a SARS-CoV-2 gene and one or more oligonucleotide compounds for inhibiting the expression of one or more of an ACE2 gene, a FURIN gene, an IL-6 gene, a TMPRSS2 gene, and a IL-6R gene.

In one aspect, the disclosure provides a pharmaceutical composition for inhibiting the expression of one or more of a SARS-CoV-2 gene, an ACE2 gene, a FURIN gene, an IL-6 gene, a TMPRSS2 gene, and a IL-6R gene in a cell of an organism, comprising the oligonucleotide compound, dsRNA, or combination recited above and a pharmaceutically acceptable carrier.

In certain embodiments, the oligonucleotide compound, dsRNA, or combination inhibits the expression of one or more of the SARS-CoV-2 genes, the ACE2 gene, the FURIN gene, the IL-6 gene, the TMPRSS2 gene, and the IL-6R gene by at least 50%. In certain embodiments, the oligonucleotide compound, dsRNA, or combination inhibits the expression of one or more of the SARS-CoV-2 genes, the ACE2 gene, the FURIN gene, the IL-6 gene, the TMPRSS2 gene, and the IL-6R gene by at least 75%.

In one aspect, the disclosure provides a pharmaceutical composition for inhibiting the expression of one or more SARS-CoV-2 genes in a cell of an organism, comprising the oligonucleotide compound, dsRNA, or combination recited above and a pharmaceutically acceptable carrier.

In certain embodiments, the oligonucleotide compound, dsRNA, or combination inhibits the expression of one or more SARS-CoV-2 genes by at least 50%. In certain embodiments, the oligonucleotide compound, dsRNA, or combination inhibits the expression of one or more SARS-CoV-2 genes by at least 75%.

In one aspect, the disclosure provides a method for inhibiting expression of a SARS-CoV-2 gene in a cell of an organism, the method comprising: (a) introducing into the cell an oligonucleotide compound, dsRNA, or combination recited above; and (b) maintaining the cell produced in step (a) for a time sufficient to obtain degradation of the mRNA transcript of the SARS-CoV-2 gene, thereby inhibiting expression of the SARS-CoV-2 gene in the cell.

In one aspect, the disclosure provides a method for inhibiting expression of one or more of a SARS-CoV-2 gene, an ACE2 gene, a FURIN gene, an IL-6 gene, a TMPRSS2 gene, and an IL-6R gene in a cell, the method comprising: (a) introducing into the cell an oligonucleotide compound, dsRNA, or combination recited above; and (b) maintaining the cell produced in step (a) for a time sufficient to obtain degradation of the mRNA transcript of the SARS-CoV-2 gene, the ACE2 gene, the FURIN gene, the IL-6 gene, the TMPRSS2 gene, and the IL-6R gene thereby inhibiting expression of the SARS-CoV-2 gene, the ACE2 gene, the FURIN gene, the IL-6 gene, the TMPRSS2 gene, and the IL-6R gene in the cell.

In one aspect, the disclosure provides a method of treating or managing a SARS-CoV-2 infection, comprising administering to a patient in need of such treatment a therapeutically effective amount of the oligonucleotide compound, dsRNA, or combination recited above.

In certain embodiments, the oligonucleotide compound, dsRNA, or combination is administered by intratracheal (IT) injection, intravenous (IV) injection, subcutaneous (SQ) injection, or a combination thereof.

In certain embodiments, the oligonucleotide compound, dsRNA, or combination is administered sequentially or simultaneously.

In certain embodiments, administering the oligonucleotide compound, dsRNA, or combination causes a decrease in one or more of SARS-CoV-2 gene mRNA, ACE2 gene mRNA, FURIN gene mRNA, IL-6 gene mRNA, TMPRSS2 gene mRNA, and IL-6R gene mRNA in the lung.

In certain embodiments, the oligonucleotide compound, dsRNA, or combination inhibits the expression of one or more of the SARS-CoV-2 gene, the ACE2 gene, the FURIN gene, the IL-6 gene, the TMPRSS2 gene, and the IL-6R gene by at least 50%. In certain embodiments, the oligonucleotide compound, dsRNA, or combination inhibits the expression of one or more of the SARS-CoV-2 gene, the ACE2 gene, the FURIN gene, the IL-6 gene, the TMPRSS2 gene, and the IL-6R gene by at least 75%.

In one aspect, the disclosure provides a vector comprising a regulatory sequence operably linked to a nucleotide sequence that encodes an oligonucleotide compound recited above.

In certain embodiments of the vector, the oligonucleotide compound inhibits the expression of one or more of the SARS-CoV-2 gene, the ACE2 gene, the FURIN gene, the IL-6 gene, the TMPRSS2 gene, and the IL-6R gene by at least 30%. In certain embodiments of the vector, the oligonucleotide compound inhibits the expression of one or more of the SARS-CoV-2 gene, the ACE2 gene, the FURIN gene, the IL-6 gene, the TMPRSS2 gene, and the IL-6R gene by at least 50%. In certain embodiments of the vector, the oligonucleotide compound inhibits the expression of one or more of the SARS-CoV-2 gene, the ACE2 gene, the FURIN gene, the IL-6 gene, the TMPRSS2 gene, and the IL-6R gene by at least 75%.

In one aspect, the disclosure provides a cell comprising the vector recited above.

In one aspect, the disclosure provides a recombinant adeno-associated virus (rAAV) comprising the vector recited above and an AAV capsid.

In one aspect, the disclosure provides a branched oligonucleotide compound comprising two or more of the oligonucleotide compounds or dsRNA recited above covalently bound to one another.

In certain embodiments, the oligonucleotide compounds are covalently bound to one another by way of a linker, spacer, a branching point, or a mixture thereof.

In one aspect, the disclosure provides a method of treating or managing a SARS-CoV-2 infection, comprising administering to a patient in need of such treatment a therapeutically effective amount of the branched oligonucleotide compound recited above.

In certain embodiments, the branched oligonucleotide compound is administered by intratracheal (IT) injection, intravenous (IV) injection, subcutaneous (SQ) injection, or a combination thereof.

In certain embodiments, the branched oligonucleotide compound accumulates in lung tissue to a greater extent than a non-branched oligonucleotide compound when administered by intratracheal (IT) injection.

In one aspect, the disclosure provides a branched RNA compound comprising: two or more RNA molecules comprising 15 to 35 nucleotides in length, and a sequence substantially complementary to a SARS-CoV-2 nucleic acid sequence, wherein the two or more RNA molecules are connected to one another by one or more moieties independently selected from a linker, a spacer and a branching point.

In certain embodiments, the branched RNA compound comprises a sequence substantially complementary to a SARS-CoV-2 nucleic acid sequence of any one of the 45-nucleotide target gene regions in Table 6A.

In certain embodiments of the branched RNA compound, each RNA molecule comprises 15 to 25 nucleotides in length.

In certain embodiments of the branched RNA compound, each RNA molecule comprises a dsRNA comprising a sense strand and an antisense strand, wherein each antisense strand independently comprises a sequence substantially complementary to a SARS-CoV-2 nucleic acid sequence of any one of the 45-nucleotide target gene regions in Table 6A.

In certain embodiments, the branched RNA compound comprises complementarity to at least 10, 11, 12 or 13 contiguous nucleotides of a SARS-CoV-2 nucleic acid sequence of any one of the 45-nucleotide target gene regions in Table 6A. In certain embodiments of the branched RNA compound, each RNA molecule comprises no more than 3 mismatches with a SARS-CoV-2 nucleic acid sequence of any one of the 45-nucleotide target gene regions in Table 6A. In certain embodiments, the branched RNA compound comprises full complementary to a SARS-CoV-2 nucleic acid sequence of any one of the 45-nucleotide target gene regions in Table 6A.

In certain embodiments of the branched RNA compound, the antisense strand comprises a portion having the nucleic acid sequence of any one of the antisense strands recited in Table 6B.

In certain embodiments of the branched RNA compound, the antisense strand and/or sense strand comprises about 15 nucleotides to 25 nucleotides in length. In certain embodiments of the branched RNA compound, the antisense strand is 20 nucleotides in length, 21 nucleotides in length, or 22 nucleotides in length. In certain embodiments of the branched RNA compound, the sense strand is 15 nucleotides in length, 16 nucleotides in length, 18 nucleotides in length, or 20 nucleotides in length.

In certain embodiments of the branched RNA compound, the dsRNA comprises a double-stranded region of 15 base pairs to 20 base pairs. In certain embodiments of the branched RNA compound, the dsRNA comprises a double-stranded region of 15 base pairs, 16 base pairs, 18 base pairs, or 20 base pairs.

In certain embodiments of the branched RNA compound, the dsRNA comprises a blunt-end. In certain embodiments of the branched RNA compound, the dsRNA comprises at least one single stranded nucleotide overhang. In certain embodiments of the branched RNA compound, the dsRNA comprises between a 2-nucleotide to 5-nucleotide single stranded nucleotide overhang.

In certain embodiments of the branched RNA compound, the dsRNA comprises naturally occurring nucleotides.

In certain embodiments of the branched RNA compound, the dsRNA comprises at least one modified nucleotide.

In certain embodiments of the branched RNA compound, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, or a non-natural base comprising nucleotide.

In certain embodiments of the branched RNA compound, the dsRNA comprises at least one modified internucleotide linkage. In certain embodiments of the branched RNA compound, the modified internucleotide linkage comprises a phosphorothioate internucleotide linkage. In certain embodiments, the branched RNA compound comprises 4-16 phosphorothioate internucleotide linkages. In certain embodiments, the branched RNA compound comprises 6-13 phosphorothioate internucleotide linkages.

In certain embodiments of the branched RNA compound, the dsRNA comprises at least one modified internucleotide linkage of Formula I:

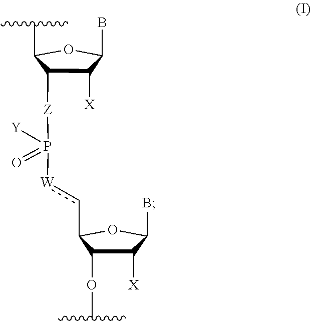

wherein:
B is a base pairing moiety;
W is selected from the group consisting of O, OCH₂, OCH, CH₂, and CH;
X is selected from the group consisting of halo, hydroxy, and C₁₋₆ alkoxy;
Y is selected from the group consisting of O⁻, OH, OR, NH⁻, NH₂, S⁻, and SH;
Z is selected from the group consisting of O and CH₂;
R is a protecting group; and
[symbol] is an optional double bond.

In certain embodiments of the branched RNA compound, the dsRNA comprises at least 80% chemically modified nucleotides. In certain embodiments of the branched RNA compound, the dsRNA is fully chemically modified. In certain embodiments of the branched RNA compound, the dsRNA comprises at least 70% 2′-O-methyl nucleotide modifications. In certain embodiments of the branched RNA compound, the antisense strand comprises at least 70% 2′-O-methyl nucleotide modifications. In certain embodiments of the branched RNA compound, the antisense strand comprises about 70% to 90% 2′-O-methyl nucleotide modifications. In certain embodiments of the branched RNA compound, the sense strand comprises at least 65% 2′-O-methyl nucleotide modifications. In certain embodiments of the branched RNA compound, the sense strand comprises 100% 2′-O-methyl nucleotide modifications.

In certain embodiments of the branched RNA compound, the sense strand comprises one or more nucleotide mismatches between the antisense strand and the sense strand. In certain embodiments of the branched RNA compound, the one or more nucleotide mismatches are present at positions 2, 6, and 12 from the 5′ end of sense strand. In certain embodiments of the branched RNA compound, the nucleotide mismatches are present at positions 2, 6, and 12 from the 5′ end of the sense strand.

In certain embodiments of the branched RNA compound, the antisense strand comprises a 5′ phosphate, a 5′-alkyl phosphonate, a 5′ alkylene phosphonate, a 5′ alkenyl phosphonate, or a mixture thereof. In certain embodiments of the branched RNA compound, the antisense strand comprises a 5′ vinyl phosphonate.

In certain embodiments of the branched RNA compound, the dsRNA comprises an antisense strand and a sense strand, each strand with a 5′ end and a 3′ end, wherein: (1) the antisense strand comprises a sequence substantially complementary to a SARS-CoV-2 nucleic acid sequence of any one of the 45-nucleotide target gene regions in Table 6A; (2) the antisense strand comprises at least 70% 2′-O-methyl modifications; (3) the nucleotides at positions 2, 6, 14, and 16 from the 5′ end of the antisense strand are not 2′-methoxy-ribonucleotides; (4) the nucleotides at positions 1-2 to 1-7 from the 3′ end of the antisense strand are connected to each other via phosphorothioate internucleotide linkages; (5) a portion of the antisense strand is complementary to a portion of the sense strand; (6) the sense strand comprises at least 70% 2′-O-methyl modifications; (7) the nucleotides at positions 7, 9, 10, and 11 from the 3′ end of the sense strand are not 2′-methoxy-ribonucleotides; and (8) the nucleotides at positions 1-2 from the 5′ end of the sense strand are connected to each other via phosphorothioate internucleotide linkages.

In certain embodiments of the branched RNA compound, the antisense strand comprises a 5′ vinyl phosphonate.

In certain embodiments of the branched RNA compound, the antisense strand is 20 nucleotides in length, 21 nucleotides in length, or 22 nucleotides in length. In certain embodiments of the branched RNA compound, the sense strand is 15 nucleotides in length, 16 nucleotides in length, 18 nucleotides in length, or 20 nucleotides in length.

In certain embodiments of the branched RNA compound, a functional moiety is linked to one or both of the 5′ end and 3′ end of the antisense strand. In certain embodiments of the branched RNA compound, a functional moiety is linked to one or both of the 5′ end and 3′ end of the sense strand. In certain embodiments of the branched RNA compound, a functional moiety is linked to the 3′ end of the sense strand.

In certain embodiments of the branched RNA compound, the functional moiety comprises a hydrophobic moiety. In certain embodiments of the branched RNA compound, the hydrophobic moiety is selected from the group consisting of fatty acids, steroids, secosteroids, lipids, gangliosides, nucleoside analogs, endocannabinoids, vitamins, and a mixture thereof. In certain embodiments of the branched RNA compound, the steroid is selected from the group consisting of cholesterol and Lithocholic acid (LCA). In certain embodiments of the branched RNA compound, the fatty acid is selected from the group consisting of Eicosapentaenoic acid (EPA), Docosahexaenoic acid (DHA) and Docosanoic acid (DCA). In certain embodiments of the branched RNA compound, the vitamin is selected from the group consisting of choline, vitamin A, vitamin E, derivatives thereof, and metabolites thereof. In certain embodiments of the branched RNA compound, the vitamin is selected from the group consisting of retinoic acid and alpha-tocopheryl succinate.

In certain embodiments of the branched RNA compound, the functional moiety is linked to one or both of the antisense strand and sense strand by a linker. In certain embodiments of the branched RNA compound, the linker comprises a divalent or trivalent linker.

In certain embodiments of the branched RNA compound, the divalent or trivalent linker is selected from the group consisting of:

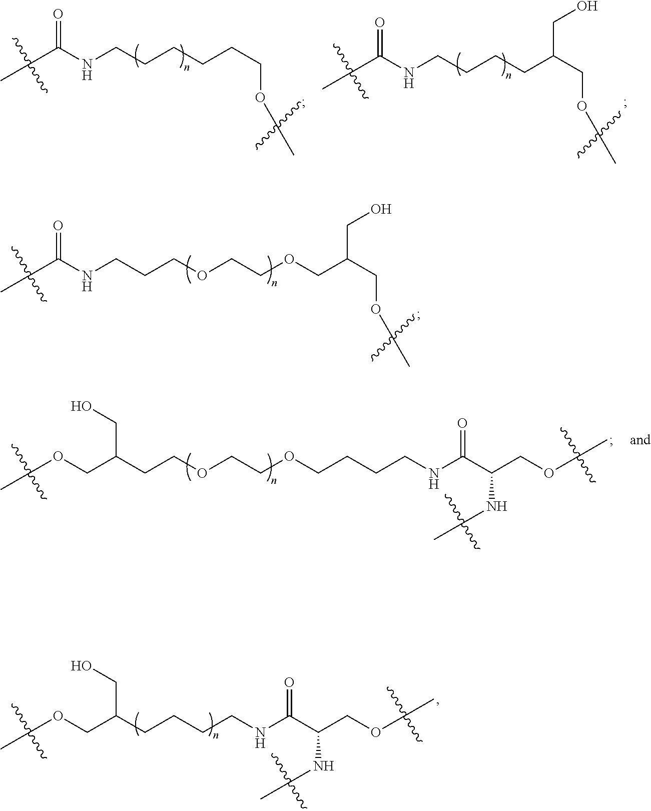

wherein n is 1, 2, 3, 4, or 5.

In certain embodiments of the branched RNA compound, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments of the branched RNA compound, the trivalent linker further links a phosphodiester or phosphodiester derivative. In certain embodiments of the branched RNA compound, the phosphodiester or phosphodiester derivative is selected from the group consisting of:

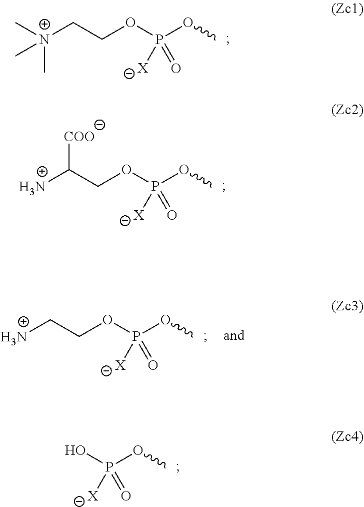

wherein X is O, S or BH₃.

In certain embodiments of the branched RNA compound, the nucleotides at positions 1 and 2 from the 3′ end of sense strand, and the nucleotides at positions 1 and 2 from the 5′ end of antisense strand, are connected to adjacent ribonucleotides via phosphorothioate linkages.

In one aspect, the disclosure provides compound of formula (I):

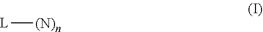

wherein:
L comprises an ethylene glycol chain, an alkyl chain, a peptide, an RNA, a DNA, a phosphate, a phosphonate, a phosphoramidate, an ester, an amide, a triazole, or combinations thereof, and wherein formula (I) optionally further comprises one or more branch point B, and one or more spacer S, wherein
B is independently for each occurrence a polyvalent organic species or derivative thereof;
S comprises independently for each occurrence an ethylene glycol chain, an alkyl chain, a peptide, an RNA, a DNA, a phosphate, a phosphonate, a phosphoramidate, an ester, an amide, a triazole, or a combination thereof; and
N is a double stranded nucleic acid comprising 15 to 35 bases in length comprising a sense strand and an antisense strand; wherein
the antisense strand comprises a sequence substantially complementary to a SARS-CoV-2 nucleic acid sequence of any one of the 45-nucleotide target gene regions in Table 6A;
the sense strand and antisense strand each independently comprise one or more chemical modifications; and
n is 2, 3, 4, 5, 6, 7 or 8.

In certain embodiments, the compound has a structure selected from formulas (I-1)-(I-9):

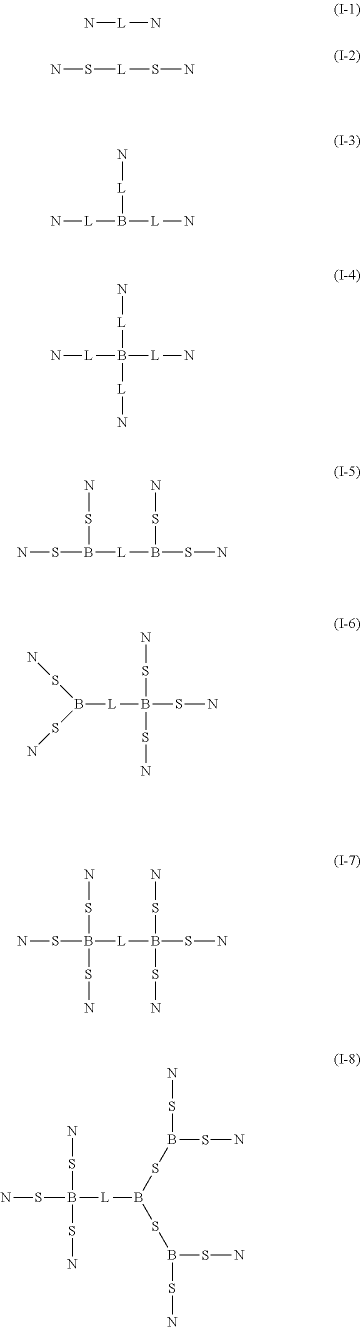

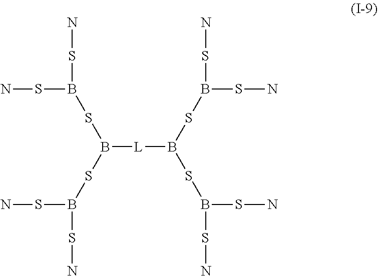

In certain embodiments, the antisense strand comprises a 5′ terminal group R selected from the group consisting of:

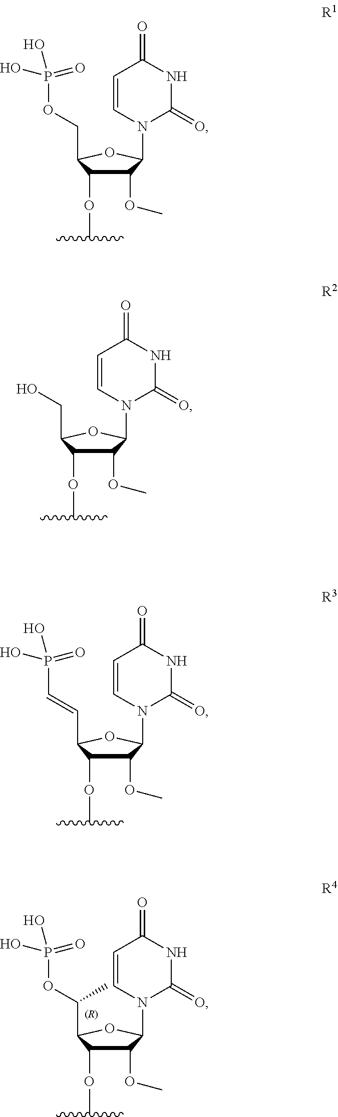

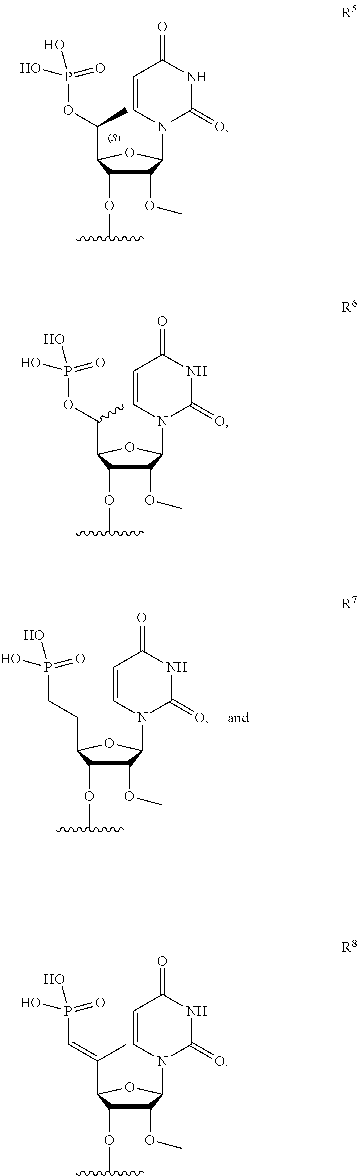

In certain embodiments, the compound comprises the structure of formula (II):

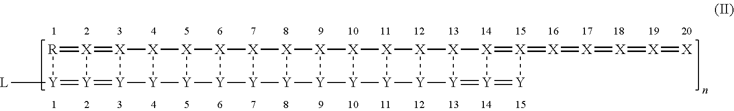

wherein:
X, for each occurrence, independently, is selected from adenosine, guanosine, uridine, cytidine, and chemically-modified derivatives thereof;
Y, for each occurrence, independently, is selected from adenosine, guanosine, uridine, cytidine, and chemically-modified derivatives thereof;
— represents a phosphodiester internucleoside linkage;
═ represents a phosphorothioate internucleoside linkage; and
[symbol] represents, individually for each occurrence, a base-pairing interaction or a mismatch.

In certain embodiments, the compound comprises structure of formula (IV):

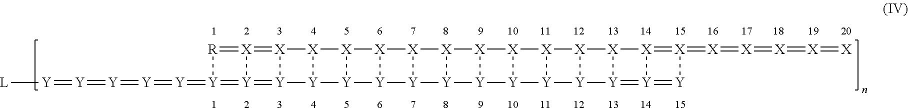

wherein:
X, for each occurrence, independently, is selected from adenosine, guanosine, uridine, cytidine, and chemically-modified derivatives thereof;
Y, for each occurrence, independently, is selected from adenosine, guanosine, uridine, cytidine, and chemically-modified derivatives thereof;
— represents a phosphodiester internucleoside linkage;
═ represents a phosphorothioate internucleoside linkage; and
[symbol] represents, individually for each occurrence, a base-pairing interaction or a mismatch.

In certain embodiments, L is structure L1:

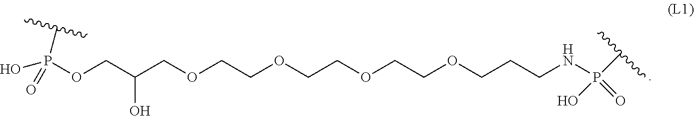 [SMILES: *P(=O)(O)NCCCOCCOCCOCCOCC(O)COP(*)(=O)O]

In certain embodiments, R is R³ and n is 2.

In certain embodiments, L is structure L2:

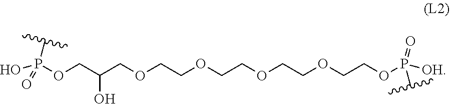 [SMILES: *P(=O)(O)OCCOCCOCCOCCOCC(O)COP(*)(=O)O]

In certain embodiments, R is R³ and n is 2.

In one aspect, the disclosure provides a delivery system for therapeutic nucleic acids having the structure of Formula (VI):

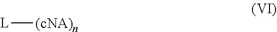

wherein:
L comprises an ethylene glycol chain, an alkyl chain, a peptide, an RNA, a DNA, a phosphate, a phosphonate, a phosphoramidate, an ester, an amide, a triazole, or combinations thereof,
wherein formula (VI) optionally further comprises one or more branch point B, and one or more spacer S, wherein
B comprises independently for each occurrence a polyvalent organic species or a derivative thereof;
S comprises independently for each occurrence an ethylene glycol chain, an alkyl chain, a peptide, RNA, DNA, a phosphate, a phosphonate, a phosphoramidate, an ester, an amide, a triazole, or combinations thereof;
each cNA, independently, is a carrier nucleic acid comprising one or more chemical modifications;
each cNA, independently, comprises at least 15 contiguous nucleotides of a SARS-CoV-2 nucleic acid sequence of any one of the 45-nucleotide target gene regions in Table 6A; and
n is 2, 3, 4, 5, 6, 7 or 8.

In certain embodiments, the delivery system has a structure selected from formulas (VI-1)-(VI-9):

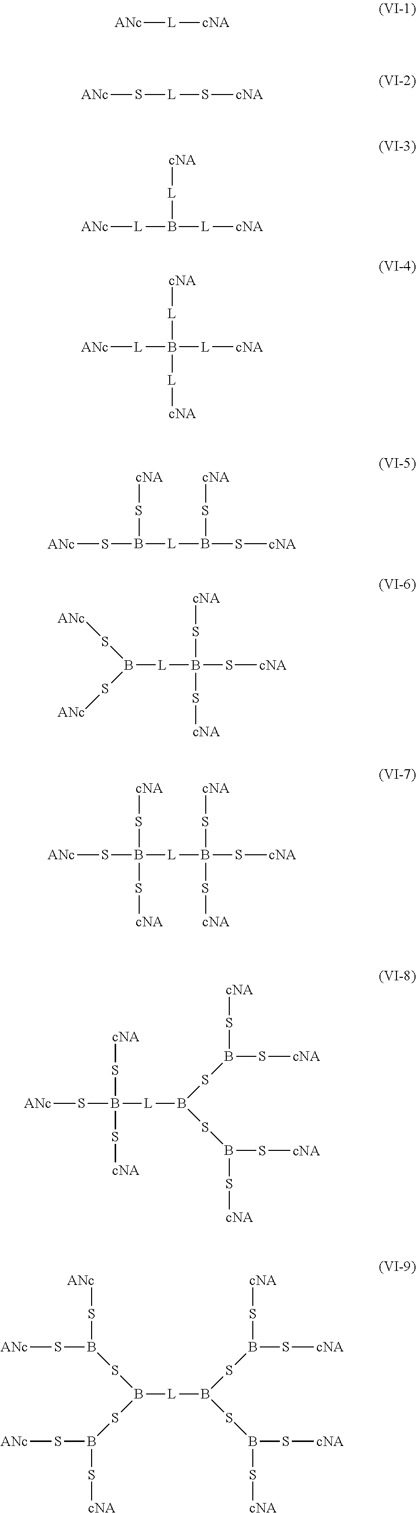

In certain embodiments, each cNA independently comprises a chemically-modified nucleotide.

In certain embodiments, the delivery system further comprises n therapeutic nucleic acids (NA), wherein each NA is hybridized to at least one cNA.

In certain embodiments, each NA independently comprises at least 16 contiguous nucleotides. In certain embodiments, each NA independently comprises 16-20 contiguous nucleotides.

In certain embodiments, each NA comprises an unpaired overhang of at least 2 nucleotides. In certain embodiments, the nucleotides of the overhang are connected via phosphorothioate linkages.

In certain embodiments, each NA, independently, is selected from the group consisting of DNAs, siRNAs, antagomiRs, miRNAs, gapmers, mixmers, and guide RNAs.

In certain embodiments, each NA is substantially complementary to a SARS-CoV-2 nucleic acid sequence of any one of the 45-nucleotide target gene regions in Table 6A.

In one aspect, the disclosure provides a pharmaceutical composition for inhibiting the expression of a SARS-CoV-2 gene in an organism, comprising a compound or a system recited above, and a pharmaceutically acceptable carrier.

In certain embodiments, the compound or system inhibits the expression of the SARS-CoV-2 gene by at least 50%. In certain embodiments, the compound or system inhibits the expression of the SARS-CoV-2 gene by at least 75%.

In one aspect, the disclosure provides a method for inhibiting expression of a SARS-CoV-2 gene in a cell, the method comprising: (a) introducing into the cell a compound or a system recited above; and (b) maintaining the cell produced in step (a) for a time sufficient to obtain degradation of the mRNA transcript of the SARS-CoV-2 gene, thereby inhibiting expression of the SARS-CoV-2 gene in the cell.

In one aspect, the disclosure provides a method of treating or managing a SARS-CoV-2 infection comprising administering to a patient in need of such treatment or management a therapeutically effective amount of a compound or a system recited above.

In certain embodiments, the compound or system is administered to the lung of the patient.

In certain embodiments, the compound or system is administered by intratracheal (IT) injection, intravenous (IV) injection, subcutaneous (SQ) injection, or a combination thereof.

In certain embodiments, administering the compound or system causes a decrease in SARS-CoV-2 gene mRNA in one or more of the club cells and alveoli cells of the lung.

In certain embodiments, the compound or system inhibits the expression of the SARS-CoV-2 gene by at least 50%. In certain embodiments, the compound or system inhibits the expression of the SARS-CoV-2 gene by at least 75%.

In certain embodiments, the compound or system accumulates in lung tissue to a greater extent than a non-branched oligonucleotide compound when administered by intratracheal (IT) injection.

In one aspect, the disclosure provides a method of delivering an oligonucleotide compound to the lung of a patient, comprising administering the oligonucleotide compound, wherein the oligonucleotide compound is conjugated to a functional moiety selected from Eicosapentaenoic acid (EPA) and Docosanoic acid (DCA).

In certain embodiments, the oligonucleotide compound is a dsRNA comprising an antisense strand and a sense strand, each strand with a 5′ end and a 3′ end. In certain embodiments, the dsRNA is conjugated to the sense strand 3′ end.

In certain embodiments, the functional moiety is conjugated to the sense strand by a linker. In certain embodiments, the linker comprises a divalent or trivalent linker.

In certain embodiments, the divalent or trivalent linker is selected from the group consisting of:

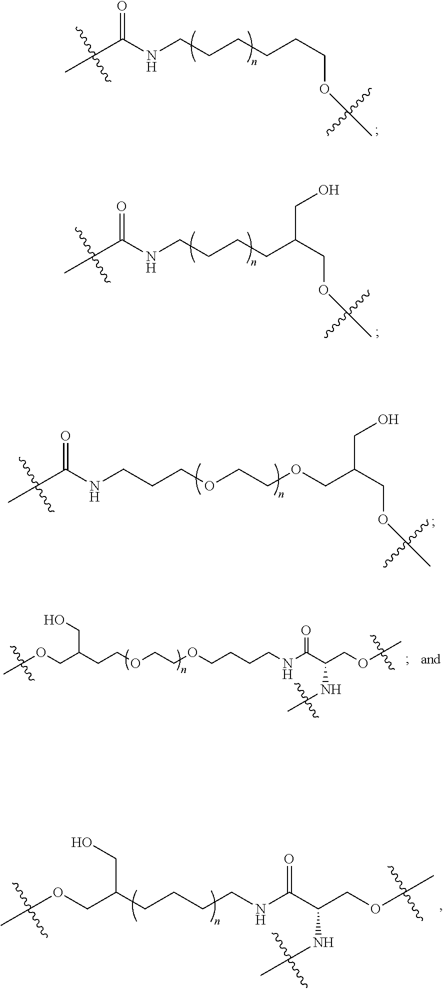

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, the trivalent linker further links a phosphodiester or phosphodiester derivative.

In certain embodiments, the phosphodiester or phosphodiester derivative is selected from the group consisting of:

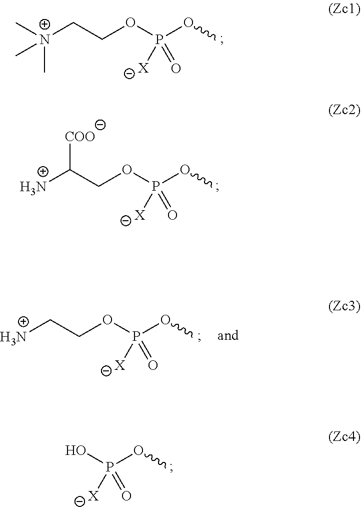

wherein X is O, S or BH₃.

### BRIEF DESCRIPTION OF THE DRAWINGS

The foregoing and other features and advantages of the present disclosure will be more fully understood from the following detailed description of illustrative embodiments taken in conjunction with the accompanying drawings.

FIG. 1A-1B depict an exemplary chemically modified siRNA (FIG. 1A), and a schematic of the SARS-CoV-2 genome (FIG. 1B).

FIG. 2 depicts a diagram of siRNA and ASO target positions on encoded proteins in the SARS-CoV-2 Genome. siRNAs were designed to target nine genes encoding SARS-CoV-2 proteins: orf1a, orf1ab, spike surface glycoprotein (S), small envelope protein (E), matrix protein (M), nucleocapsid protein (N), and accessory proteins 3a, 8b, 7a. Grey arrows indicate siRNA and ASO target positions. Inset shows detailed view of siRNA target positions on genes in the 3′ region of the genome.

FIG. 3A-3B depict an alignment of siRNAs and ASOs selected for synthesis directed to six closely-related CoVs using a novel algorithm. Aligned genome regions of CoVs are shaded based on homology with darker coloring indicating higher homology with respect to SARS-CoV-2. The siRNA position is indicated on the top. Per position percent homology of SARS-CoV-2 to the six related CoVs is plotted on the bottom. SiRNA with low homology scores of 59 are shown in FIG. 3A (SEQ ID NOS 2583-2589, respectively, in order of appearance). SiRNA with a high homology score are shown in FIG. 3B (SEQ ID NOS 2590-2596, respectively, in order of appearance). Gaps in alignment are indicated with dashes (-).

FIG. 4A-4B depict siRNA and ASO target selection based on the ability to target many SARS-CoV-2 genomes from patient isolates. FIG. 4A, siRNAs and ASOs were selected to target regions of the 9 selection genes with low mutation rates in other coronaviruses. FIG. 4B, the proportion of SARS-CoV-2 variants from patient isolates targeted by all selected siRNAs. FIG. FIG. 5A-5I depicts the identification of siRNA hits for SARS-CoV-2. SiRNAs targeting different genes in the SARS-CoV-2 genome were tested for silencing efficacy.

FIG. 5A, gene orf1a, FIG. 5B, gene 3a; FIG. 5C, gene 7a, FIG. 5D, gene orf1ab, FIG. 5E, gene E, FIG. 5F, gene 8b, FIG. 5G, gene S, FIG. 5H, gene M, FIG. 5I, gene N. SiRNAs were tested in Hela cells and silencing was assessed using the psi-check reporter system. Concentration: 1.5 uM; Time point: 72 hours.

FIG. 6A-6I depict the identification of ASO hits for SARS-CoV-2. ASOs targeting different genes in the SARS-CoV2 genome were tested for silencing efficacy. FIG. 6A, gene orf1a, FIG. 6B, gene Orf1ab; FIG. 6C, gene S, FIG. 6D, gene 3a, FIG. 6E, gene E, FIG. 6F, gene M, FIG. 6G, gene 7a, FIG. 6H, gene 8b, FIG. 61, gene N. ASOs were tested in Hela cells and silencing was assessed using the psi-check reporter system. Concentration: 1.5 uM; Time point: 72 hours.

FIG. 7A-7B depict the identification of siRNA hits for SARS-CoV2 and mapping onto genes in the SARS-CoV-2 genome. FIG. 7A, SARS-CoV-2 genome. FIG. 7B, siRNAs targeting different genes in the SARS-CoV2 genome tested for silencing efficacy. siRNAs were tested in Hela cells and silencing was assessed using the psi-check reporter system. Concentration: 1.5 uM; Time point: 72 hours.

FIG. 8A-8B depicts the identification of ASO hits for SARS-CoV-2 and mapping onto genes in the SARS-CoV-2 genome. FIG. 8A, SARS-CoV-2 genome. FIG. 8B, LNA gapmers targeting different genes in the SARS-CoV-2 genome were tested for silencing efficacy. ASOs were tested in Hela cells and silencing was assessed using the psi-check reporter system. Concentration: 1.5 μM; Time point: 72 hours.

FIG. 9A-9I depict the validation and determination of IC₅₀ values. SiRNAs targeting different genes in the SARS-CoV-2 genome were tested for silencing efficacy in 8-point dose response studies. FIG. 9A, gene orf1a, FIG. 9B, gene Orf1ab; FIG. 9C, gene Spike, FIG. 9D, gene 3a, FIG. 9E, gene Envelope, FIG. 9F, gene Membrane, FIG. 9G, gene Orf7a, FIG. 9H, gene Orf8a, FIG. 91, gene Nucleocapsid. siRNAs were tested in Hela cells and silencing was assessed using the psi-check reporter system. Concentration: Top=1.5 μM; Time point: 72 hours.

FIG. 10 depicts a schematic showing SARS-CoV-2 genes and their functions. Non-structural genes undergo primary translation while structural and accessory proteins are translated from sub-genomic mRNAs.

FIG. 11A-11E depict validation and determination of IC₅₀ values for siRNA cocktails targeting SARS-CoV-2 Genes. FIG. 11A, replication cocktails; FIG. 11B, Replication/immuno cocktails; FIG. 11C, Replication/Capsid cocktails; FIG. 11D, Immuno/Capsid cocktails; and FIG. 11E, Replication/Immuno/Capsid cocktails. siRNAs were tested in Hela cells and silencing was assessed using the psi-check reporter system. Concentration: Top=1.5 uM; Time point: 72 hours.

FIG. 12A-12B depict design of siRNAs targeting ACE2 (FIG. 12A) and FURIN (FIG. 12B).

FIG. 13A-13B depict the identification of siRNA hits for ACE2 (FIG. 13A) and FURIN (FIG. 13B). siRNAs were tested in human Hacat cells and silencing was assessed using the QuantiGene assay and confirmed using psicheck reporter system. Concentration: 1.5 μM; Time point: 72 hours.

FIG. 14A-14B depict validation and determination of IC₅₀ values for siRNAs targeting ACE2 (FIG. 14A) and FURIN (FIG. 14B). siRNAs were tested in Hela cells and silencing was assessed using the psi-check reporter system. Concentration: Top=1.5 μM; Time point: 72 hours.

FIG. 15A-15D depict validation and determination of IC₅₀ values for siRNAs targeting FURIN. FIG. 15A, 2711; FIG. 15B, 2712; FIG. 15C, 3524; FIG. 15D, 3526. siRNAs were tested in HaCat cells and silencing was assessed using QuantiGene. Concentration: Top=1.5 μM; Time point: 72 hours.

FIG. 16A-16B depict the identification of ASO hits for ACE2 and FURIN. Twelve LNA gapmers targeting ACE2 (FIG. 16A) and FURIN (FIG. 16B) were tested for silencing efficacy. ASOs were tested in human U2OS cells and silencing was assessed using QRT-PCR assay. Concentration: 1.5 μM; Time point: 72 hours.

FIG. 17A-17B depict the dentification of ASO hits for ACE2 and FURIN. Twelve LNA gapmers targeting ACE2 (FIG. 17A) and FURIN (FIG. 17B), were tested for silencing efficacy. ASOs were tested in human U2OS cells and silencing was assessed using QRT-PCR assay. Concentration: 1.5 μM; Time point: 72 hours.

FIG. 18A-18B depict validation and determination of IC₅₀ values for ASOs targeting ACE2 (FIG. 18A) and FURIN (FIG. 18B). Concentration: Top=1.5 μM; Time point: 5 days.

FIG. 19A-19F show that the presence of a two-thymidine linker between the conjugate and the siRNA does not impact siRNA tissue distribution profile. The siRNA structural configurations studied to evaluate the impact of the nature of the linker on distribution is shown in FIG. 19A, FIG. 19C, and FIG. 19E. The corresponding bar graphs for siRNA accumulation in the various tissues are shown in FIG. 19B, FIG. 19D, and FIG. 19F, respectively. Shown are siRNA strand accumulation of DCA-conjugated siRNA in liver, kidney, spleen, lung, heart, muscle and fat 1-week after a single SC injection with 20 mg/kg (n=5-6 mice per group±SD), measured by PNA hybridization assay.

FIG. 20A-20B show that the presence of a two-thymidine linker increases DCA-conjugated siRNA silencing in multiple tissues. FIG. 20A, effect on huntingtin mRNA expression. FIG. 20B, effect on cyclophilin B mRNA expression. The presence of a two-thymidine linker increases DCA-conjugated siRNA silencing in multiple tissues. SC injection (FVB/N mice); 20 mg/kg; collection of tissues one week after injection; n=6 per group. Huntingtin (Htt) (A.) or Cyclophilin B (Ppib) (B.) mRNA levels were measured using QuantiGene® (Affymetrix), normalized to a housekeeping gene, Hprt (Hypoxanthine-guanine phosphoribosyl transferase), and presented as percent of PBS (Phosphate buffered saline) control (mean±SD). Data analysis: t test (****P<0.0001, ***P<0.001, **P<0.01, *P<0.1).

FIG. 21 depicts designs and structural configurations of siRNAs for lung delivery via systemic (SC). Schematic of siRNA structural configuration studied to evaluate the impact of the chemical composition on siRNA distribution and efficacy.

FIG. 22 depicts distribution and accumulation of siRNAs conjugated to DCA and containing different numbers of 3′ exNA modifications and phosphorothioates. These data show increased accumulation of DCA-conjugated siRNAs with exNA modifications compared to those without exNAs in all tissues including the lungs. SC, 20 mg/kg, n=3, 1 week, PNA hybridization assay. p2 scaffold: 4PS-exNa≈7PS in liver, spleen, lung; 4PS-exNa >7 PS in heart, muscle, fat; 7PS >4PS-exNa in kidney. p5 scaffold: 4PS-exNa >2PO-exNa 2PS-exNa ≥4PS>2PS.

FIG. 23A-23H depict target mRNA silencing (Htt) after systemic administration of siRNAs conjugated to DCA and containing different numbers of 3′ exNA modifications and phosphorothioates in various organ tissues. FIG. 23A, liver; FIG. 23B, kidney; FIG. 23C, spleen; FIG. 23D, muscle; FIG. 23E, lung; FIG. 23F, heart; FIG. 23G, adrenal glands; FIG. 23H, Fat. These data show increased silencing of DCA-conjugated siRNAs with exNA modifications compared to those without exNAs in all tissues including the lungs. SC, 20 mg/kg, n=5, 1 week, bDNA QuantiGene assay.

FIG. 24 depicts designs of siRNAs for lung delivery via intratracheal administration. Schematic of siRNA structural configuration studied to evaluate the impact of the chemical composition on siRNA distribution and efficacy.

FIG. 25A-25B depict siRNA accumulation after intratracheal administration. FIG. 25A, Distribution and delivery throughout the lung of mono and divalent siRNAs (Cy-3) compared to PBS controls; FIG. 25B, distribution and delivery throughout the lung of EPA and DCA conjugated siRNAs (Cy-3) compared to PBS controls. Intratracheal; 20 nmol for mono, 40 nmol for di; n=2, 24 h, 5×, Scale=1 mm. Subcutaneous; 40 nmol; n=3, 48 h, 5×, Scale=1 mm.

FIG. 26A-26C depict mono and di-valent siRNA accumulation after intratracheal administration. FIG. 26A, distribution and delivery throughout the lung of mono and divalent siRNAs (Cy-3) compared to PBS controls; FIG. 26B, distribution and delivery throughout the lung of mono and divalent siRNAs (Red, Cy3) to club cells (epithelial) (green) of the lungs compared to PBS controls; FIG. 26C, distribution and delivery throughout the lung of mono and divalent siRNAs (Red, Cy3) to alveoli type II cells (green) in the lung compared to PBS controls. Divalent siRNAs distribute to all cells of the lungs and saturate both alveolar and epithelial (club) cells 24 hours after intratracheal administration.

FIG. 27A-27C depict EPA and DCA conjugated siRNA distribution throughout the lungs after subcutaneous (SC) administration). FIG. 27A, distribution and delivery throughout the lung of EPA and DCA conjugated siRNAs (Cy-3) compared to PBS controls; FIG. 27B, distribution of EPA and DCA conjugated siRNAs (Red, Cy3) to club cells (epithelial) (green) of the lungs compared to PBS controls; FIG. 27C, distribution of EPA and DCA conjugated siRNAs (Red, Cy3) to alveoli cells (green) in the lung compared to PBS controls.

FIG. 28A-28D depict quantification of siRNA accumulation after systemic and intratracheal administration. FIG. 28A, fluorescence uptake in alveolar cells; FIG. 28B, percent in alveoli cells/total cells; FIG. 28C, fluorescence uptake in club cells; FIG. 28D, percent in club cells/total cells.

FIG. 29A-29G depict quantification of EPA and DCA conjugated siRNA accumulation after systemic administration. FIG. 29A, distribution in various lung cells; FIG. 29B, distribution by cell type; FIG. 29C-29G, CY3 signals in total cells, immune cells, endothelial cells, epithelial cells and fibroblasts, respectively. Using flow cytometry siRNA accumulation was quantified after systemic (SC) administration of EPA and DCA conjugated siRNAs.

FIG. 30A-30G depict quantification of siRNA accumulation after intratracheal administration. Using flow cytometry, siRNA accumulation was quantified after intratracheal administration of mono and di-valent siRNAs. FIG. 30A, distribution in various lung cells;

FIG. 30B, distribution by cell type; FIG. 30C-30G, CY3 signals in total cells, immune cells, endothelial cells, epithelial cells and fibroblasts, respectively. Divalent siRNAs showed the highest amount of uptake in all cell types compared to other siRNAs.

FIG. 31A-31B depict distribution and accumulation of mono and di-siRNAs. FIG. 31A, di-siRNAs after intratracheal administration; FIG. 31B, di-siRNAs after intratracheal administration and DAC/EPA siRNA after SC injection. Intratracheal or SC, 7.5 and 15 nmol and 40 nmol, n=3, 1 week, PNA hybridization assay.

FIG. 32A-32H show target mRNA silencing (Htt) after intratracheal administration of mono and di-siRNAs in various tissues. FIG. 32A, liver; FIG. 32B, kidney;

FIG. 32C, spleen; FIG. 32D, lung; FIG. 32E, heart, FIG. 32F, adrenal glands; FIG. 32G, muscle; FIG. 32H, fat. Low dose of di-siRNA achieved the best silencing in lungs without silence the gene in other tissues. Intratracheal, 7.5 or 15 nmol, n=5, 1 week, bDNA QuantiGene assay.

FIG. 33 depicts target mRNA silencing (Htt) after intratracheal administration of mono and di-siRNAs. Intratracheal, 7.5 or 15 nmol, n=5, 1 week, bDNA QuantiGene assay.

FIG. 34 depicts a screen of siRNAs and ASOs targeting various SARS-CoV2 genes and tested for silencing efficacy. siRNAs and ASOs were tested in in A549-ACE2 cells and silencing was assessed using the psi-check reporter system. siRNA concentration: 10 nM; ASO concentration: 25 nM; Time point: 72 hours.

FIG. 35 depicts dose response data of select siRNAs targeting various SARS-CoV2 genes. The data reports relative mRNA abundance of the targeted SARS-CoV2 genes and the percent of cells that are postive for the SARS-CoV2 spike protein. siRNAs were tested in in A549-ACE2 cells and silencing was assessed using the psi-check reporter system.

FIG. 36 depicts dose response data of select siRNAs targeting various SARS-CoV2 genes. The A549-ACE2 cells were infected with SARS-CoV-2 at a MOI of 0.1 and 0.4. The data reports relative mRNA abundance of the targeted SARS-CoV2 genes.

FIG. 37 depicts a screen of siRNAs targeting the orf7a SARS-CoV2 gene. siRNAs were tested in in A549-ACE2 cells and the data reports relative mRNA abundance of the targeted orf7a SARS-CoV2 gene and the percent of cells that are postive for the SARS-CoV2 spike protein. siRNA concentration: 10 nM; Time point: 72 hours.

### DETAILED DESCRIPTION

Novel SARS-CoV-2 target sequences are provided. Also provided are novel RNA molecules, such as siRNAs and branched RNA compounds containing the same, that target one or more SARS-CoV-2 genes mRNA, such as one or more target sequences of the disclosure. Also provided are novel ACE2, FURIN, IL-6, and TMPRSS2 target sequences.

Unless otherwise specified, nomenclature used in connection with cell and tissue culture, molecular biology, immunology, microbiology, genetics and protein and nucleic acid chemistry and hybridization described herein are those well-known and commonly used in the art. Unless otherwise specified, the methods and techniques provided herein are performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “of” means “and/or” unless stated otherwise. The use of the term “including,” as well as other forms, such as “includes” and “included,” is not limiting.

So that the invention may be more readily understood, certain terms are first defined.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide, which may be derivatized include: the 5 position, e.g., 5-(2-amino)propyl uridine, 5-bromo uridine, 5-propyne uridine, 5-propenyl uridine, etc.; the 6 position, e.g., 6-(2-amino)propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs, such as those described in Herdewijn, Antisense Nucleic Acid Drug Dev., 2000 Aug. 10(4):297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, or COOR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions, which allow the nucleotide to perform its intended function, such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “oligonucleotide” refers to a short polymer of nucleotides and/or nucleotide analogs.

The term “RNA analog” refers to a polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Some RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An RNAi agent, e.g., an RNA silencing agent, having a strand, which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules, which result in the inhibition or “silencing” of the expression of a corresponding protein-coding gene. RNA silencing has been observed in many types of organisms, including plants, animals, and fungi.

The term “discriminatory RNA silencing” refers to the ability of an RNA molecule to substantially inhibit the expression of a “first” or “target” polynucleotide sequence while not substantially inhibiting the expression of a “second” or “non-target” polynucleotide sequence,” e.g., when both polynucleotide sequences are present in the same cell. In certain embodiments, the target polynucleotide sequence corresponds to a target gene, while the non-target polynucleotide sequence corresponds to a non-target gene. In other embodiments, the target polynucleotide sequence corresponds to a target allele, while the non-target polynucleotide sequence corresponds to a non-target allele. In certain embodiments, the target polynucleotide sequence is the DNA sequence encoding the regulatory region (e.g. promoter or enhancer elements) of a target gene. In other embodiments, the target polynucleotide sequence is a target mRNA encoded by a target gene.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the term “transgene” refers to any nucleic acid molecule, which is inserted by artifice into a cell, and becomes part of the genome of the organism that develops from the cell. Such a transgene may include a gene that is partly or entirely heterologous (i.e., foreign) to the transgenic organism, or may represent a gene homologous to an endogenous gene of the organism. The term “transgene” also means a nucleic acid molecule that includes one or more selected nucleic acid sequences, e.g., DNAs, that encode one or more engineered RNA precursors, to be expressed in a transgenic organism, e.g., animal, which is partly or entirely heterologous, i.e., foreign, to the transgenic animal, or homologous to an endogenous gene of the transgenic animal, but which is designed to be inserted into the animal's genome at a location which differs from that of the natural gene. A transgene includes one or more promoters and any other DNA, such as introns, necessary for expression of the selected nucleic acid sequence, all operably linked to the selected sequence, and may include an enhancer sequence.

A gene “involved” in a disease or disorder includes a gene, the normal or aberrant expression or function of which effects or causes the disease or disorder or at least one symptom of said disease or disorder.

The term “gain-of-function mutation” as used herein, refers to any mutation in a gene in which the protein encoded by said gene (i.e., the mutant protein) acquires a function not normally associated with the protein (i.e., the wild type protein) and causes or contributes to a disease or disorder. The gain-of-function mutation can be a deletion, addition, or substitution of a nucleotide or nucleotides in the gene, which gives rise to the change in the function of the encoded protein. In one embodiment, the gain-of-function mutation changes the function of the mutant protein or causes interactions with other proteins. In another embodiment, the gain-of-function mutation causes a decrease in or removal of normal wild-type protein, for example, by interaction of the altered, mutant protein with said normal, wild-type protein.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g. mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g. an orthologue or paralogue) of the target gene.

As described herein, the term “SARS-CoV-2” refers to the severe acute respiratory syndrome coronavirus 2, which can cause severe respiratory illness. The SARS-CoV-2 genome contains nine genes. Four genes encode for four structural proteins, S (spike), E (envelope), M (matrix) and N (nucleocapsid). The five other genes are orf1a, orf1ab, 3a, 8b, and 7a. The sequence of the SARS-CoV-2 and the nine genes are recited in Table 1 and Table 2, respectively.

The phrase “examining the function of a gene in a cell or organism” refers to examining or studying the expression, activity, function or phenotype arising therefrom.

As used herein, the term “RNA silencing agent” refers to an RNA, which is capable of inhibiting or “silencing” the expression of a target gene. In certain embodiments, the RNA silencing agent is capable of preventing complete processing (e.g., the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small noncoding RNAs can be generated. Exemplary RNA silencing agents include siRNAs, miRNAs, siRNA-like duplexes, antisense oligonucleotides, GAPMER molecules, and dual-function oligonucleotides, as well as precursors thereof. In one embodiment, the RNA silencing agent is capable of inducing RNA interference. In another embodiment, the RNA silencing agent is capable of mediating translational repression.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, 2N-methylguanosine and 2,2N,N-dimethylguanosine.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by a human. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

As used herein, the term “microRNA” (“miRNA”), also known in the art as “small temporal RNAs” (“stRNAs”), refers to a small (10-50 nucleotide) RNA, which are genetically encoded (e.g., by viral, mammalian, or plant genomes) and are capable of directing or mediating RNA silencing. An “miRNA disorder” shall refer to a disease or disorder characterized by an aberrant expression or activity of a miRNA.

As used herein, the term “dual functional oligonucleotide” refers to a RNA silencing agent having the formula T-L-p, wherein T is an mRNA targeting moiety, L is a linking moiety, and p is a miRNA recruiting moiety. As used herein, the terms “mRNA targeting moiety,” “targeting moiety,” “mRNA targeting portion” or “targeting portion” refer to a domain, portion or region of the dual functional oligonucleotide having sufficient size and sufficient complementarity to a portion or region of an mRNA chosen or targeted for silencing (i.e., the moiety has a sequence sufficient to capture the target mRNA).

As used herein, the term “linking moiety” or “linking portion” refers to a domain, portion or region of the RNA-silencing agent which covalently joins or links the mRNA.

As used herein, the term “antisense strand” of an RNA silencing agent, e.g., an siRNA or RNA silencing agent, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target mRNA.

As used herein, the term “asymmetry,” as in the asymmetry of the duplex region of an RNA silencing agent (e.g., the stem of an shRNA), refers to an inequality of bond strength or base pairing strength between the termini of the RNA silencing agent (e.g., between terminal nucleotides on a first strand or stem portion and terminal nucleotides on an opposing second strand or stem portion), such that the 5′ end of one strand of the duplex is more frequently in a transient unpaired, e.g., single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, van der Waals interactions, and the like, between said nucleotides (or nucleotide analogs).

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein the term “destabilizing nucleotide” refers to a first nucleotide or nucleotide analog capable of forming a base pair with second nucleotide or nucleotide analog such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of a RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g. certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portion of universal nucleotides typically comprise a nitrogen-containing aromatic heterocyclic moiety.

As used herein, the terms “sufficient complementarity” or “sufficient degree of complementarity” mean that the RNA silencing agent has a sequence (e.g. in the antisense strand, mRNA targeting moiety or miRNA recruiting moiety), which is sufficient to bind the desired target RNA, respectively, and to trigger the RNA silencing of the target mRNA.

As used herein, the term “translational repression” refers to a selective inhibition of mRNA translation. Natural translational repression proceeds via miRNAs cleaved from shRNA precursors. Both RNAi and translational repression are mediated by RISC. Both RNAi and translational repression occur naturally or can be initiated by the hand of man, for example, to silence the expression of target genes.

Various methodologies of the instant invention include a step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

As used herein, the terms “extended nucleic acid” or “exNA” or ex-NA” refer to a novel oligonucleotide backbone modification. This chemical modification of the backbone significantly enhances oligonucleotide metabolic stability. The chemical modification includes one or more carbon atoms or chains inserted in the backbone at the 5′-position, 3′-position, or both. This structural modulation forms non-canonical stretched/flexible structure on oligo-backbones, which protect oligonucleotides from cleavage by various nucleases.

The novel exNA-modification is widely compatible in any oligonucleotide, such as an siRNA, antisense oligonucleotide, and mRNA. The combination of an exNA-phosphorothioate (exNA-PS) backbone enables drastic enhancement of metabolic stability (10-50 orders of magnitude as compared to unmodified oligonucleotides) without compromising the function of the oligonucleotide (e.g., siRNA-mediated silencing efficacy). For example, 5′-[exNA-PS]4-3′ modification induce NO negative impact on siRNA efficacy while inducing drastically high exonuclease stability, as will be shown below. Moreover, an exNA-phosphodiester (exNA-PO) backbone also enables drastic enhancement of metabolic stability without compromising the function of the oligonucleotide. It has been previously shown that phosphorothioate-containing backbones in oligonucleotides are toxic when administered in vivo. Accordingly, the exNA-PO backbone can be employed to enhancement of metabolic stability while decreasing toxicity. Thus, this metabolically stabilizing exNA modification is widely and robustly improves the performance of therapeutic oligonucleotide candidates in vivo.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and example are illustrative only and not intended to be limiting.

Various aspects of the invention are described in further detail in the following subsections.

### I. Novel Target Sequences

In certain exemplary embodiments, RNA silencing agents of the invention are capable of targeting a SARS-CoV-2 nucleic acid sequence of any one of SEQ ID NOs: 1-10, as recited in Table 4 and Table 5.

In certain exemplary embodiments, RNA silencing agents of the invention are capable of targeting one or more of a SARS-CoV-2 nucleic acid sequence of any one of the 45-nucleotide target gene regions recited in Table 6A. In certain exemplary embodiments, RNA silencing agents of the invention are capable of targeting one or more of a SARS-CoV-2 nucleic acid sequence of any one of the 20-nucleotide target regions recited in Table 6A.

In certain exemplary embodiments, RNA silencing agents of the invention are capable of targeting one or more of a SARS-CoV-2 nucleic acid sequence of any one of the 45-nucleotide target gene regions recited in Table 7. In certain exemplary embodiments, RNA silencing agents of the invention are capable of targeting one or more of a SARS-CoV-2 nucleic acid sequence of any one of the 20-nucleotide target regions recited in Table 7.

In certain exemplary embodiments, RNA silencing agents of the invention are capable of targeting one or more of a SARS-CoV-2 nucleic acid sequence of any one of the 45-nucleotide target gene regions recited in Table 8. In certain exemplary embodiments, RNA silencing agents of the invention are capable of targeting one or more of a SARS-CoV-2 nucleic acid sequence of any one of the 20-nucleotide target regions recited in Table 8.

In certain exemplary embodiments, RNA silencing agents of the invention comprise an antisense strand as recited in Table 6B. In certain exemplary embodiments, RNA silencing agents of the invention comprise a sense strand as recited in Table 6C. In certain exemplary embodiments, RNA silencing agents of the invention comprise a sense strand as recited in Table 6D.

In one embodiment, the RNA silencing agents of the invention capable of targeting one or more of a SARS-CoV-2 nucleic acid sequence can be combined with RNA silencing agents of the invention capable of targeting one or more of an ACE2, FURIN, TMPRSS2, IL-6, and IL-6R nucleic acid sequence. The endogenous genes ACE2, FURIN, TMPRSS2, IL-6, and IL-6R each may play a role in SARS-CoV-2 infection and pathogenesis.

“ACE2”, as described herein, refers to angiotensin I converting enzyme 2. ACE2 belongs to a family of dipeptidyl carboxydipeptidases and is a zinc-containing metallo protease. ACE2 cleaves angiotensin I into angiotensin II, which has vasoconstrictive properties. It also is a functional receptor of the spike glycoprotein of the human corona viruses. ACE2 expression is age and disease state dependent. Children have lower ACE2 expression, which may explain their decreased susceptibility and milder disease symptoms upon SARS-CoV-2 infection. As such, a reduction in ACE2 expression is a viable therapeutic approach.

“FURIN”, as described herein, refers to a subtilisin-like proprotein belonging to the convertase family of proteases. They include proteases that process protein and peptides precursors as they traffic through the constitute branches of the secretory pathway. Furin is exploited by viruses for cleaving envelope proteins. The spike protein of the SARS-CoV-2 virus must be cleaved by furin to become functional, and as such furin represents an attractive target for siRNA (Coutard et al. Antiviral Research. 176:104727. April 2020).

“TMPRSS2”, as described herein, refers to Transmembrane Serine Protease 2. TMPRSS2 has been shown to contribute to virus spread and immunopathology in the airways of murine models after coronavirus infection (Iwata-Yoshikawa et al. J. Virol. 93(6):e01815-18. March 2019).

“IL-6”, as described herein, refers to interleukin-6. “IL-6R”, as described herein, refers to interlukin-6 receptor. IL-6 is an inflammatory agent that contributes to cytokine release syndrome (CRS), a severe and potentially deadly response to an infection. IL-6 stimulates inflammation through an interaction with IL-6R (Liu et al. J Autoimmun. 10: 102452. April 2020). Inhibition of IL-6 or its receptor, IL-6R may prevent or reduce the cytokine release syndrome that occurs during a SARS-CoV-2 infection.

In certain exemplary embodiments, RNA silencing agents of the invention are capable of targeting an ACE2 nucleic acid sequence of any one of the 45-nucleotide target gene regions recited in Table 11D and Table 12C. In certain exemplary embodiments, RNA silencing agents of the invention are capable of targeting an ACE2 nucleic acid sequence of any one of the 20-nucleotide target regions recited in Table 11D and Table 12C.

In certain exemplary embodiments, RNA silencing agents of the invention are capable of targeting an FURIN nucleic acid sequence of any one of the 45-nucleotide target gene regions recited in Table 11C and Table 12D. In certain exemplary embodiments, RNA silencing agents of the invention are capable of targeting an FURIN nucleic acid sequence of any one of the 20-nucleotide target regions recited in Table 11C and Table 12D.

In certain exemplary embodiments, RNA silencing agents of the invention are capable of targeting an TMPRSS2 nucleic acid sequence of any one of the 45-nucleotide target gene regions recited in Table 11A and Table 12A. In certain exemplary embodiments, RNA silencing agents of the invention are capable of targeting an TMPRSS2 nucleic acid sequence of any one of the 20-nucleotide target regions recited in Table 11A and Table 12A.

In certain exemplary embodiments, RNA silencing agents of the invention are capable of targeting an IL-6 nucleic acid sequence of any one of the 45-nucleotide target gene regions recited in Table 11B and Table 12B. In certain exemplary embodiments, RNA silencing agents of the invention are capable of targeting an IL-6 nucleic acid sequence of any one of the 20-nucleotide target regions recited in Table 11B and Table 12B.

In certain exemplary embodiments, RNA silencing agents of the invention are capable of targeting an IL-6R nucleic acid sequence of any one of the 45-nucleotide target gene regions recited in Table 12E. In certain exemplary embodiments, RNA silencing agents of the invention are capable of targeting an IL-6R nucleic acid sequence of any one of the 20-nucleotide target regions recited in Table 12E.

Genomic sequence for each target sequence can be found in, for example, the publicly available database maintained by the NCBI.

II. siRNA Design

In some embodiments, siRNAs are designed as follows. First, a portion of the target gene (e.g., the SARS-CoV-2 gene), e.g., one or more of the target sequences set forth in Table 4, Table 5, Table 6A, Table 7, or Table 8 is selected. Cleavage of mRNA at these sites should eliminate translation of corresponding protein. Antisense strands were designed based on the target sequence and sense strands were designed to be complementary to the antisense strand. Hybridization of the antisense and sense strands forms the siRNA duplex. The antisense strand includes about 19 to 25 nucleotides, e.g., 19, 20, 21, 22, 23, 24 or 25 nucleotides. In other embodiments, the antisense strand includes 20, 21, 22 or 23 nucleotides. The sense strand includes about 14 to 25 nucleotides, e.g., 14, 15, 16, 17, 18, 19, 20, 21, 22, 23, 24 or 25 nucleotides. In other embodiments, the sense strand is 15 nucleotides. In other embodiments, the sense strand is 18 nucleotides. In other embodiments, the sense strand is 20 nucleotides. The skilled artisan will appreciate, however, that siRNAs having a length of less than 19 nucleotides or greater than 25 nucleotides can also function to mediate RNAi. Accordingly, siRNAs of such length are also within the scope of the instant invention, provided that they retain the ability to mediate RNAi. Longer RNAi agents have been demonstrated to elicit an interferon or PKR response in certain mammalian cells, which may be undesirable. In certain embodiments, the RNAi agents of the invention do not elicit a PKR response (i.e., are of a sufficiently short length). However, longer RNAi agents may be useful, for example, in cell types incapable of generating a PKR response or in situations where the PKR response has been down-regulated or dampened by alternative means.

The sense strand sequence can be designed such that the target sequence is essentially in the middle of the strand. Moving the target sequence to an off-center position can, in some instances, reduce efficiency of cleavage by the siRNA. Such compositions, i.e., less efficient compositions, may be desirable for use if off-silencing of the wild-type mRNA is detected.

The antisense strand can be the same length as the sense strand and includes complementary nucleotides. In one embodiment, the strands are fully complementary, i.e., the strands are blunt-ended when aligned or annealed. In another embodiment, the strands align or anneal such that 1-, 2-, 3-, 4-, 5-, 6-, 7-, or 8-nucleotide overhangs are generated, i.e., the 3′ end of the sense strand extends 1, 2, 3, 4, 5, 6, 7, or 8 nucleotides further than the 5′ end of the antisense strand and/or the 3′ end of the antisense strand extends 1, 2, 3, 4, 5, 6, 7, or 8 nucleotides further than the 5′ end of the sense strand. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material.

To facilitate entry of the antisense strand into RISC (and thus increase or improve the efficiency of target cleavage and silencing), the base pair strength between the 5′ end of the sense strand and 3′ end of the antisense strand can be altered, e.g., lessened or reduced, as described in detail in U.S. Pat. Nos. 7,459,547, 7,772,203 and 7,732,593, entitled “Methods and Compositions for Controlling Efficacy of RNA Silencing” (filed Jun. 2, 2003) and U.S. Pat. Nos. 8,309,704, 7,750,144, 8,304,530, 8,329,892 and 8,309,705, entitled “Methods and Compositions for Enhancing the Efficacy and Specificity of RNAi” (filed Jun. 2, 2003), the contents of which are incorporated in their entirety by this reference. In one embodiment of these aspects of the invention, the base-pair strength is less due to fewer G:C base pairs between the 5′ end of the first or antisense strand and the 3′ end of the second or sense strand than between the 3′ end of the first or antisense strand and the 5′ end of the second or sense strand. In another embodiment, the base pair strength is less due to at least one mismatched base pair between the 5′ end of the first or antisense strand and the 3′ end of the second or sense strand. In certain exemplary embodiments, the mismatched base pair is selected from the group consisting of G:A, C:A, C:U, G:G, A:A, C:C and U:U. In another embodiment, the base pair strength is less due to at least one wobble base pair, e.g., G:U, between the 5′ end of the first or antisense strand and the 3′ end of the second or sense strand. In another embodiment, the base pair strength is less due to at least one base pair comprising a rare nucleotide, e.g., inosine (I). In certain exemplary embodiments, the base pair is selected from the group consisting of an I:A, I:U and I:C. In yet another embodiment, the base pair strength is less due to at least one base pair comprising a modified nucleotide. In certain exemplary embodiments, the modified nucleotide is selected from the group consisting of 2-amino-G, 2-amino-A, 2,6-diamino-G, and 2,6-diamino-A.

The design of siRNAs suitable for targeting the SARS-CoV-2 target sequences set forth in Table 4, Table 5, Table 6A, Table 7, or Table 8 is described in detail below. siRNAs can be designed according to the above exemplary teachings for any other target sequences found in the SARS-CoV-2 gene. Moreover, the technology is applicable to targeting any other target sequences, e.g., non-disease-causing target sequences.

To validate the effectiveness by which siRNAs destroy mRNAs (e.g., SARS-CoV-2 mRNA), the siRNA can be incubated with cDNA (e.g., SARS-CoV-2 cDNA) in a Drosophila-based in vitro mRNA expression system. Radiolabeled with ³²P, newly synthesized mRNAs (e.g., SARS-CoV-2 mRNA) are detected autoradiographically on an agarose gel. The presence of cleaved mRNA indicates mRNA nuclease activity. Suitable controls include omission of siRNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence. Sites of siRNA-mRNA complementation are selected which result in optimal mRNA specificity and maximal mRNA cleavage.

### III. RNAi Agents

The present invention includes RNAi molecules, such as siRNA molecules designed, for example, as described above. The siRNA molecules of the invention can be chemically synthesized, or can be transcribed in vitro from a DNA template, or in vivo from e.g., shRNA, or by using recombinant human DICER enzyme, to cleave in vitro transcribed dsRNA templates into pools of 20-, 21- or 23-bp duplex RNA mediating RNAi. The siRNA molecules can be designed using any method known in the art.

In one aspect, instead of the RNAi agent being an interfering ribonucleic acid, e.g., an siRNA or shRNA as described above, the RNAi agent can encode an interfering ribonucleic acid, e.g., an shRNA, as described above. In other words, the RNAi agent can be a transcriptional template of the interfering ribonucleic acid. Thus, RNAi agents of the present invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21-23 nucleotides (Brummelkamp et al., 2002; Lee et al., 2002, Supra; Miyagishi et al., 2002; Paddison et al., 2002, supra; Paul et al., 2002, supra; Sui et al., 2002 supra; Yu et al., 2002, supra. More information about shRNA design and use can be found on the internet at the following addresses: katandin.cshl.org:9331/RNAi/docs/BseRI-BamHI_Strategy.pdf and katandin.cshl.org:9331/RNAi/docs/Web_version_of_PCR_strategy1.pdf).

Expression constructs of the present invention include any construct suitable for use in the appropriate expression system and include, but are not limited to, retroviral vectors, linear expression cassettes, plasmids and viral or virally-derived vectors, as known in the art. Such expression constructs can include one or more inducible promoters, RNA Pol III promoter systems, such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct. (Tuschl, T., 2002, Supra).

Synthetic siRNAs can be delivered into cells by methods known in the art, including cationic liposome transfection and electroporation. To obtain longer term suppression of the target genes (e.g., SARS-CoV-2 genes) and to facilitate delivery under certain circumstances, one or more siRNA can be expressed within cells from recombinant DNA constructs. Such methods for expressing siRNA duplexes within cells from recombinant DNA constructs to allow longer-term target gene suppression in cells are known in the art, including mammalian Pol III promoter systems (e.g., H1 or U6/snRNA promoter systems (Tuschl, T., 2002, supra) capable of expressing functional double-stranded siRNAs; (Bagella et al., 1998; Lee et al., 2002, supra; Miyagishi et al., 2002, supra; Paul et al., 2002, supra; Yu et al., 2002, supra; Sui et al., 2002, supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al., 1998; Lee et al., 2002, supra; Miyagishi et al., 2002, supra; Paul et al., 2002, supra; Yu et al., 2002), supra; Sui et al., 2002, supra). Constructs containing siRNA sequence under the control of T7 promoter also make functional siRNAs when co-transfected into the cells with a vector expressing T7 RNA polymerase (Jacque et al., 2002, supra). A single construct may contain multiple sequences coding for siRNAs, such as multiple regions of the gene encoding SARS-CoV-2, targeting the same gene or multiple genes, and can be driven, for example, by separate PolIII promoter sites.

Animal cells express a range of noncoding RNAs of approximately 22 nucleotides termed micro RNA (miRNAs), which can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with sequence complementary to the target mRNA, a vector construct that expresses the engineered precursor can be used to produce siRNAs to initiate RNAi against specific mRNA targets in mammalian cells (Zeng et al., 2002, supra). When expressed by DNA vectors containing polymerase III promoters, micro-RNA designed hairpins can silence gene expression (McManus et al., 2002, supra). MicroRNAs targeting polymorphisms may also be useful for blocking translation of mutant proteins, in the absence of siRNA-mediated gene-silencing. Such applications may be useful in situations, for example, where a designed siRNA caused off-target silencing of wild type protein.

Viral-mediated delivery mechanisms can also be used to induce specific silencing of targeted genes through expression of siRNA, for example, by generating recombinant adenoviruses harboring siRNA under RNA Pol II promoter transcription control (Xia et al., 2002, supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., 2002). In adult mice, efficient delivery of siRNA can be accomplished by “high-pressure” delivery technique, a rapid injection (within 5 seconds) of a large volume of siRNA containing solution into animal via the tail vein (Liu et al., 1999, supra; McCaffrey et al., 2002, supra; Lewis et al., 2002. Nanoparticles and liposomes can also be used to deliver siRNA into animals. In certain exemplary embodiments, recombinant adeno-associated viruses (rAAVs) and their associated vectors can be used to deliver one or more siRNAs into cells, e.g., lung (e.g., endothelial cells, epithelial cells, fibroblasts, and immune cells in the lungs, e.g. clara cells, alveolar cells, and club cells)

The nucleic acid compositions of the invention include both unmodified siRNAs and modified siRNAs, such as crosslinked siRNA derivatives or derivatives having non-nucleotide moieties linked, for example to their 3′ or 5′ ends. Modifying siRNA derivatives in this way may improve cellular uptake or enhance cellular targeting activities of the resulting siRNA derivative, as compared to the corresponding siRNA, and are useful for tracing the siRNA derivative in the cell, or improving the stability of the siRNA derivative compared to the corresponding siRNA.

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage and destruction. In this fashion, the mRNA, which will be targeted by the siRNA generated from the engineered RNA precursor, and will be depleted from the cell or organism, leading to a decrease in the concentration of the protein encoded by that mRNA in the cell or organism. The RNA precursors are typically nucleic acid molecules that individually encode either one strand of a dsRNA or encode the entire nucleotide sequence of an RNA hairpin loop structure.

The nucleic acid compositions of the invention can be unconjugated or can be conjugated to another moiety, such as a nanoparticle, to enhance a property of the compositions, e.g., a pharmacokinetic parameter such as absorption, efficacy, bioavailability and/or half-life. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The nucleic acid molecules of the present invention can also be labeled using any method known in the art. For instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P or another appropriate isotope.

Moreover, because RNAi is believed to progress via at least one single-stranded RNA intermediate, the skilled artisan will appreciate that ss-siRNAs (e.g., the antisense strand of a ds-siRNA) can also be designed (e.g., for chemical synthesis), generated (e.g., enzymatically generated), or expressed (e.g., from a vector or plasmid) as described herein and utilized according to the claimed methodologies. Moreover, in invertebrates, RNAi can be triggered effectively by long dsRNAs (e.g., dsRNAs about 100-1000 nucleotides in length, such as about 200-500, for example, about 250, 300, 350, 400 or 450 nucleotides in length) acting as effectors of RNAi. (Brondani et al., Proc Natl Acad Sci USA. 2001 Dec. 4; 98(25):14428-33. Epub 2001 Nov. 27.)

### IV. Anti-SARS-CoV-2 RNA Silencing Agents

In certain embodiment, the present invention provides novel anti-SARS-CoV-2 RNA silencing agents (e.g., siRNA, shRNA, and antisense oligonucleotides), methods of making said RNA silencing agents, and methods (e.g., research and/or therapeutic methods) for using said improved RNA silencing agents (or portions thereof) for RNA silencing of SARS-CoV-2 protein. The RNA silencing agents comprise an antisense strand (or portions thereof), wherein the antisense strand has sufficient complementary to a target SARS-CoV-2 mRNA to mediate an RNA-mediated silencing mechanism (e.g. RNAi).

In certain embodiments, siRNA compounds are provided having one or any combination of the following properties: (1) fully chemically-stabilized (i.e., no unmodified 2′-OH residues); (2) asymmetry; (3) 11-20 base pair duplexes; (4) greater than 50% 2′-methoxy modifications, such as 70%-100% 2′-methoxy modifications, although an alternating pattern of chemically-modified nucleotides (e.g., 2′-fluoro and 2′-methoxy modifications), are also contemplated; and (5) single-stranded, fully phosphorothioated tails of 5-8 bases. In certain embodiments, the number of phosphorothioate modifications is varied from 4 to 16 total. In certain embodiments, the number of phosphorothioate modifications is varied from 8 to 13 total.

In certain embodiments, the siRNA compounds described herein can be conjugated to a variety of targeting agents, including, but not limited to, docosanoic acid (DCA), cholesterol, docosahexaenoic acid (DHA), phenyltropanes, cortisol, vitamin A, vitamin D, N-acetylgalactosamine (GalNac), and gangliosides. The cholesterol-modified version showed 5-10 fold improvement in efficacy in vitro versus previously used chemical stabilization patterns (e.g., wherein all purine but not pyrimidines are modified) in wide range of cell types (e.g., HeLa, neurons, hepatocytes, trophoblasts. lung epithelial cells).

Certain compounds of the invention having the structural properties described above and herein may be referred to as “hsiRNA-ASP” (hydrophobically-modified, small interfering RNA, featuring an advanced stabilization pattern). In addition, siRNAs conjugated to DCA or EPA and containing different numbers of 3′ exNA modifications and phosphorothioates showed a dramatically improved distribution through the lung, making them accessible for therapeutic intervention.

In certain embodiments, the siRNA comprises between 6 and 13 total phosphorothioate modifications. In certain embodiments, the siRNA comprises 6 phosphorothioate modifications. In certain embodiments, the siRNA comprises 8 phosphorothioate modifications. In certain embodiments, the siRNA antisense strand comprises 6 phosphorothioate modifications. In certain embodiments, the siRNA antisense strand comprises 4 phosphorothioate modifications. In certain embodiments, the siRNA sense strand comprises 2 phosphorothioate modifications.

In certain embodiments, the siRNA sense strand 3′ end is conjugated to DCA. In certain embodiments, the siRNA sense strand 3′ end is conjugated to EPA.

In certain embodiments, the siRNA antisense strand comprises two to five 3′ exNA modifications. In certain embodiments, the siRNA antisense strand comprises two 3′ exNA modifications. In certain embodiments, the siRNA antisense strand comprises three 3′ exNA modifications. In certain embodiments, the siRNA antisense strand comprises four 3′ exNA modifications. In certain embodiments, the siRNA antisense strand comprises five 3′ exNA modifications. In certain embodiments, the siRNA antisense strand comprises four consecutive 3′ exNA modifications.

In certain embodiments, the siRNA antisense strand comprises two to five 3′ exNA modifications and 6 phosphorothioate modifications. In certain embodiments, the siRNA antisense strand comprises two to five 3′ exNA modifications and 4 phosphorothioate modifications. In certain embodiments, the siRNA antisense strand comprises four 3′ exNA modifications and 6 phosphorothioate modifications. In certain embodiments, the siRNA antisense strand comprises four 3′ exNA modifications and 4 phosphorothioate modifications.

The compounds of the invention can be described in the following aspects and embodiments.

In a first aspect, provided herein is a double stranded RNA (dsRNA) comprising an antisense strand and a sense strand, each strand comprising at least 14 contiguous nucleotides, with a 5′ end and a 3′ end, wherein:
(1) the antisense strand comprises a sequence substantially complementary to a SARS-CoV-2 nucleic acid sequence of any one of the 45-nucleotide target gene regions in Table 6A;
(2) the antisense strand comprises alternating 2′-methoxy-ribonucleotides and 2′-fluoro-ribonucleotides;
(3) the nucleotides at positions 2 and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises alternating 2′-methoxy-ribonucleotides and 2′-fluoro-ribonucleotides; and
(7) the nucleotides at positions 1-2 from the 5′ end of the sense strand are connected to each other via phosphorothioate internucleotide linkages.

In a second aspect, provided herein is a dsRNA comprising an antisense strand and a sense strand, each strand comprising at least 14 contiguous nucleotides, with a 5′ end and a 3′ end, wherein:
(1) the antisense strand comprises a sequence substantially complementary to a SARS-CoV-2 nucleic acid sequence of any one of the 45-nucleotide target gene regions in Table 6A;
(2) the antisense strand comprises at least 70% 2′-O-methyl modifications;
(3) the nucleotide at position 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications; and
(7) the nucleotides at positions 1-2 from the 5′ end of the sense strand are connected to each other via phosphorothioate internucleotide linkages.

In a third aspect, provided herein is a dsRNA comprising an antisense strand and a sense strand, each strand comprising at least 14 contiguous nucleotides, with a 5′ end and a 3′ end, wherein:
(1) the antisense strand comprises a sequence substantially complementary to a SARS-CoV-2 nucleic acid sequence of any one of the 45-nucleotide target gene regions in Table 6A;
(2) the antisense strand comprises at least 85% 2′-O-methyl modifications;
(3) the nucleotides at positions 2 and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises 100% 2′-O-methyl modifications; and
(7) the nucleotides at positions 1-2 from the 5′ end of the sense strand are connected to each other via phosphorothioate internucleotide linkages.

In a fourth aspect, provided herein is a dsRNA comprising an antisense strand and a sense strand, each strand comprising at least 14 contiguous nucleotides, with a 5′ end and a 3′ end, wherein:
(1) the antisense strand comprises a sequence substantially complementary to a SARS-CoV-2 nucleic acid sequence of any one of the 45-nucleotide target gene regions in Table 6A;
(2) the antisense strand comprises at least 75% 2′-O-methyl modifications;
(3) the nucleotides at positions 4, 5, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises 100% 2′-O-methyl modifications; and
(7) the nucleotides at positions 1-2 from the 5′ end of the sense strand are connected to each other via phosphorothioate internucleotide linkages.

In a fifth aspect, provided herein is a dsRNA comprising an antisense strand and a sense strand, each strand comprising at least 14 contiguous nucleotides, with a 5′ end and a 3′ end, wherein:
(1) the antisense strand comprises a sequence substantially complementary to a SARS-CoV-2 nucleic acid sequence of any one of the 45-nucleotide target gene regions in Table 6A;
(2) the antisense strand comprises at least 75% 2′-O-methyl modifications;
(3) the nucleotides at positions 2, 4, 5, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises 100% 2′-O-methyl modifications; and
(7) the nucleotides at positions 1-2 from the 5′ end of the sense strand are connected to each other via phosphorothioate internucleotide linkages.

In a sixth aspect, provided herein is a dsRNA comprising an antisense strand and a sense strand, each strand comprising at least 14 contiguous nucleotides, with a 5′ end and a 3′ end, wherein:
(1) the antisense strand comprises a sequence substantially complementary to a SARS-CoV-2 nucleic acid sequence of any one of the 45-nucleotide target gene regions in Table 6A;
(2) the antisense strand comprises at least 75% 2′-O-methyl modifications;
(3) the nucleotides at positions 2, 6, 14, and 16 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 70% 2′-O-methyl modifications;
(7) the nucleotides at positions 7, 9, 10, and 11 from the 3′ end of the sense strand are not 2′-methoxy-ribonucleotides; and
(8) the nucleotides at positions 1-2 from the 5′ end of the sense strand are connected to each other via phosphorothioate internucleotide linkages.

In a seventh aspect, provided herein is a dsRNA comprising an antisense strand and a sense strand, each strand comprising at least 14 contiguous nucleotides, with a 5′ end and a 3′ end, wherein:
(1) the antisense strand comprises a sequence substantially complementary to a SARS-CoV-2 nucleic acid sequence of any one of the 45-nucleotide target gene regions in Table 6A;
(2) the antisense strand comprises at least 75% 2′-O-methyl modifications;
(3) the nucleotides at positions 2, 6, and 14 from the 5′ end of the antisense strand are not 2′-methoxy-ribonucleotides;
(4) the nucleotides at positions 1-2 to 1-7 from the 3′ end of the antisense strand are connected to each other via phosphorothioate internucleotide linkages;
(5) a portion of the antisense strand is complementary to a portion of the sense strand;
(6) the sense strand comprises at least 80% 2′-O-methyl modifications;
(7) the nucleotides at positions 7, 10, and 11 from the 3′ end of the sense strand are not 2′-methoxy-ribonucleotides; and
(8) the nucleotides at positions 1-2 from the 5′ end of the sense strand are connected to each other via phosphorothioate internucleotide linkages.
   a) Design of Anti-SARS-CoV-2 siRNA Molecules

An siRNA molecule of the application is a duplex made of a sense strand and complementary antisense strand, the antisense strand having sufficient complementary to a SARS-CoV-2 mRNA to mediate RNAi. In certain embodiments, the siRNA molecule has a length from about 10-50 or more nucleotides, i.e., each strand comprises 10-50 nucleotides (or nucleotide analogs). In other embodiments, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In certain embodiments, the strands are aligned such that there are at least 1, 2, 3, 4, 5, 6, 7, 8, 9, or 10 bases at the end of the strands, which do not align (i.e., for which no complementary bases occur in the opposing strand), such that an overhang of 1, 2, 3, 4, 5, 6, 7, 8, 9, or 10 residues occurs at one or both ends of the duplex when strands are annealed.

Usually, siRNAs can be designed by using any method known in the art, for instance, by using the following protocol:
1. The siRNA should be specific for a target sequence, e.g., a target sequence set forth in the Examples. The first strand should be complementary to the target sequence, and the other strand is substantially complementary to the first strand. (See Examples for exemplary sense and antisense strands.) Exemplary target sequences are selected from any region of the target gene that leads to potent gene silencing. Regions of the target gene include, but are not limited to, the 5′ untranslated region (5′-UTR) of a target gene, the 3′ untranslated region (3′-UTR) of a target gene, an exon of a target gene, or an intron of a target gene. Cleavage of mRNA at these sites should eliminate translation of corresponding SARS-CoV-2 protein. Target sequences from other regions of the SARS-CoV-2 gene are also suitable for targeting. A sense strand is designed based on the target sequence.
2. The sense strand of the siRNA is designed based on the sequence of the selected target site. In certain embodiments, the sense strand includes about 15 to 25 nucleotides, e.g., 15, 16, 17, 18, 19, 20, 21, 22, 23, 24 or 25 nucleotides. In certain embodiments, the sense strand includes 15, 16, 17, 18, 19, or 20 nucleotides. In certain embodiments, the sense strand is 15 nucleotides in length. In certain embodiments, the sense strand is 18 nucleotides in length. In certain embodiments, the sense strand is 20 nucleotides in length. The skilled artisan will appreciate, however, that siRNAs having a length of less than 15 nucleotides or greater than 25 nucleotides can also function to mediate RNAi. Accordingly, siRNAs of such length are also within the scope of the instant invention, provided that they retain the ability to mediate RNAi. Longer RNA silencing agents have been demonstrated to elicit an interferon or Protein Kinase R (PKR) response in certain mammalian cells which may be undesirable. In certain embodiments, the RNA silencing agents of the invention do not elicit a PKR response (i.e., are of a sufficiently short length). However, longer RNA silencing agents may be useful, for example, in cell types incapable of generating a PKR response or in situations where the PKR response has been down-regulated or dampened by alternative means.

The siRNA molecules of the invention have sufficient complementarity with the target sequence such that the siRNA can mediate RNAi. In general, siRNA containing nucleotide sequences sufficiently complementary to a target sequence portion of the target gene to effect RISC-mediated cleavage of the target gene are contemplated. Accordingly, in a certain embodiment, the antisense strand of the siRNA is designed to have a sequence sufficiently complementary to a portion of the target. For example, the antisense strand may have 100% complementarity to the target site. However, 100% complementarity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% complementarity, between the antisense strand and the target RNA sequence is contemplated. The present application has the advantage of being able to tolerate certain sequence variations to enhance efficiency and specificity of RNAi. In one embodiment, the antisense strand has 4, 3, 2, 1, or 0 mismatched nucleotide(s) with a target region, such as a target region that differs by at least one base pair between a wild-type and mutant allele, e.g., a target region comprising the gain-of-function mutation, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and the percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.
3. The antisense or guide strand of the siRNA is routinely the same length as the sense strand and includes complementary nucleotides. In one embodiment, the guide and sense strands are fully complementary, i.e., the strands are blunt-ended when aligned or annealed. In another embodiment, the strands of the siRNA can be paired in such a way as to have a 3′ overhang of 1 to 7 (e.g., 2, 3, 4, 5, 6 or 7), or 1 to 4, e.g., 2, 3 or 4 nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus, in another embodiment, the nucleic acid molecules may have a 3′ overhang of 2 nucleotides, such as TT. The overhanging nucleotides may be either RNA or DNA. As noted above, it is desirable to choose a target region wherein the mutant:wild type mismatch is a purine:purine mismatch.
4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.
5. Select one or more sequences that meet your criteria for evaluation.

Further general information about the design and use of siRNA may be found in “The siRNA User Guide,” available at The Max-Plank-Institut fur Biophysikalische Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tₘ is determined according to the following equations. For hybrids less than 18 base pairs in length, Tₘ(° C.)=2(#of A+T bases)+4(#of G+C bases). For hybrids between 18 and 49 base pairs in length, Tₘ(° C.)=81.5+16.6(log 10[Na+])+0.41(% G+C)-(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

6. To validate the effectiveness by which siRNAs destroy target mRNAs (e.g., wild-type or mutant SARS-CoV-2 mRNA), the siRNA may be incubated with target cDNA (e.g., SARS-CoV-2 cDNA) in a Drosophila-based in vitro mRNA expression system. Radiolabeled with ³²P, newly synthesized target mRNAs (e.g., SARS-CoV-2 mRNA) are detected autoradiographically on an agarose gel. The presence of cleaved target mRNA indicates mRNA nuclease activity. Suitable controls include omission of siRNA and use of non-target cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

Anti-SARS-CoV-2 siRNAs may be designed to target any of the target sequences described supra. Said siRNAs comprise an antisense strand, which is sufficiently complementary with the target sequence to mediate silencing of the target sequence. In certain embodiments, the RNA silencing agent is a siRNA.

In certain embodiments, the siRNA comprises a sense strand comprising a sequence set forth in Table 6C or Table 6D, and an antisense strand comprising a sequence set forth in Table 6B.

Sites of siRNA-mRNA complementation are selected, which result in optimal mRNA specificity and maximal mRNA cleavage.

b) siRNA-Like Molecules

siRNA-like molecules of the invention have a sequence (i.e., have a strand having a sequence) that is “sufficiently complementary” to a target sequence of an SARS-CoV-2 mRNA to direct gene silencing either by RNAi or translational repression. siRNA-like molecules are designed in the same way as siRNA molecules, but the degree of sequence identity between the sense strand and target RNA approximates that observed between a miRNA and its target. In general, as the degree of sequence identity between a miRNA sequence and the corresponding target gene sequence is decreased, the tendency to mediate post-transcriptional gene silencing by translational repression rather than RNAi is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g. within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity. In one embodiment, where gene silencing by translational repression is desired, at least one non-identical nucleotide is present in the central portion of the complementarity site so that duplex formed by the miRNA guide strand and the target mRNA contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected such that it forms a wobble base pair (e.g., G:U) or a mismatched base pair (G:A, C:A, C:U, G:G, A:A, C:C, U:U). In a further embodiment, the “bulge” is centered at nucleotide positions 12 and 13 from the 5′ end of the miRNA molecule.

c) Short Hairpin RNA (shRNA) Molecules

In certain featured embodiments, the instant invention provides shRNAs capable of mediating RNA silencing of an SARS-CoV-2 target sequence with enhanced selectivity. In contrast to siRNAs, shRNAs mimic the natural precursors of micro RNAs (miRNAs) and enter at the top of the gene silencing pathway. For this reason, shRNAs are believed to mediate gene silencing more efficiently by being fed through the entire natural gene silencing pathway.

miRNAs are noncoding RNAs of approximately 22 nucleotides, which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof. Naturally-occurring miRNA precursors (pre-miRNA) have a single strand that forms a duplex stem including two portions that are generally complementary, and a loop, that connects the two portions of the stem. In typical pre-miRNAs, the stem includes one or more bulges, e.g., extra nucleotides that create a single nucleotide “loop” in one portion of the stem, and/or one or more unpaired nucleotides that create a gap in the hybridization of the two portions of the stem to each other. Short hairpin RNAs, or engineered RNA precursors, of the present application are artificial constructs based on these naturally occurring pre-miRNAs, but which are engineered to deliver desired RNA silencing agents (e.g., siRNAs of the invention). By substituting the stem sequences of the pre-miRNA with sequence complementary to the target mRNA, a shRNA is formed. The shRNA is processed by the entire gene silencing pathway of the cell, thereby efficiently mediating RNAi.

The requisite elements of a shRNA molecule include a first portion and a second portion, having sufficient complementarity to anneal or hybridize to form a duplex or double-stranded stem portion. The two portions need not be fully or perfectly complementary. The first and second “stem” portions are connected by a portion having a sequence that has insufficient sequence complementarity to anneal or hybridize to other portions of the shRNA. This latter portion is referred to as a “loop” portion in the shRNA molecule. The shRNA molecules are processed to generate siRNAs. shRNAs can also include one or more bulges, i.e., extra nucleotides that create a small nucleotide “loop” in a portion of the stem, for example a one-, two- or three-nucleotide loop. The stem portions can be the same length, or one portion can include an overhang of, for example, 1-5 nucleotides. The overhanging nucleotides can include, for example, uracils (Us), e.g., all Us. Such Us are notably encoded by thymidines (Ts) in the shRNA-encoding DNA which signal the termination of transcription.

In shRNAs (or engineered precursor RNAs) of the instant invention, one portion of the duplex stem is a nucleic acid sequence that is complementary (or anti-sense) to the SARS-CoV-2 target sequence. In certain embodiments, one strand of the stem portion of the shRNA is sufficiently complementary (e.g., antisense) to a target RNA (e.g., mRNA) sequence to mediate degradation or cleavage of said target RNA via RNA interference (RNAi). Thus, engineered RNA precursors include a duplex stem with two portions and a loop connecting the two stem portions. The antisense portion can be on the 5′ or 3′ end of the stem. The stem portions of a shRNA are about 15 to about 50 nucleotides in length. In certain embodiments, the two stem portions are about 18 or 19 to about 21, 22, 23, 24, 25, 30, 35, 37, 38, 39, or 40 or more nucleotides in length. In certain embodiments, the length of the stem portions should be 21 nucleotides or greater. When used in mammalian cells, the length of the stem portions should be less than about 30 nucleotides to avoid provoking non-specific responses like the interferon pathway. In non-mammalian cells, the stem can be longer than 30 nucleotides. In fact, the stem can include much larger sections complementary to the target mRNA (up to, and including the entire mRNA). In fact, a stem portion can include much larger sections complementary to the target mRNA (up to, and including the entire mRNA).

The two portions of the duplex stem must be sufficiently complementary to hybridize to form the duplex stem. Thus, the two portions can be, but need not be, fully or perfectly complementary. In addition, the two stem portions can be the same length, or one portion can include an overhang of 1, 2, 3, or 4 nucleotides. The overhanging nucleotides can include, for example, uracils (Us), e.g., all Us. The loop in the shRNAs or engineered RNA precursors may differ from natural pre-miRNA sequences by modifying the loop sequence to increase or decrease the number of paired nucleotides, or replacing all or part of the loop sequence with a tetraloop or other loop sequences. Thus, the loop in the shRNAs or engineered RNA precursors can be 2, 3, 4, 5, 6, 7, 8, 9, or more, e.g., 15 or 20, or more nucleotides in length.

The loop in the shRNAs or engineered RNA precursors may differ from natural pre-miRNA sequences by modifying the loop sequence to increase or decrease the number of paired nucleotides, or replacing all or part of the loop sequence with a tetraloop or other loop sequences. Thus, the loop portion in the shRNA can be about 2 to about 20 nucleotides in length, i.e., about 2, 3, 4, 5, 6, 7, 8, 9, or more, e.g., 15 or 20, or more nucleotides in length. In certain embodiments, a loop consists of or comprises a “tetraloop” sequence. Exemplary tetraloop sequences include, but are not limited to, the sequences GNRA, where N is any nucleotide and R is a purine nucleotide, GGGG, and UUUU.

In certain embodiments, shRNAs of the present application include the sequences of a desired siRNA molecule described supra. In other embodiments, the sequence of the antisense portion of a shRNA can be designed essentially as described above or generally by selecting an 18, 19, 20, 21 nucleotides, or longer, sequence from within the target RNA (e.g., SARS-CoV-2 mRNA), for example, from a region 100 to 200 or 300 nucleotides upstream or downstream of the start of translation. In general, the sequence can be selected from any portion of the target RNA (e.g., mRNA) including the 5′ UTR (untranslated region), coding sequence, or 3′ UTR. This sequence can optionally follow immediately after a region of the target gene containing two adjacent AA nucleotides. The last two nucleotides of the nucleotide sequence can be selected to be UU. This 21 or so nucleotide sequence is used to create one portion of a duplex stem in the shRNA. This sequence can replace a stem portion of a wild-type pre-miRNA sequence, e.g., enzymatically, or is included in a complete sequence that is synthesized. For example, one can synthesize DNA oligonucleotides that encode the entire stem-loop engineered RNA precursor, or that encode just the portion to be inserted into the duplex stem of the precursor, and using restriction enzymes to build the engineered RNA precursor construct, e.g., from a wild-type pre-miRNA.

Engineered RNA precursors include, in the duplex stem, the 21-22 or so nucleotide sequences of the siRNA or siRNA-like duplex desired to be produced in vivo. Thus, the stem portion of the engineered RNA precursor includes at least 18 or 19 nucleotide pairs corresponding to the sequence of an exonic portion of the gene whose expression is to be reduced or inhibited. The two 3′ nucleotides flanking this region of the stem are chosen so as to maximize the production of the siRNA from the engineered RNA precursor and to maximize the efficacy of the resulting siRNA in targeting the corresponding mRNA for translational repression or destruction by RNAi in vivo and in vitro.

In certain embodiments, shRNAs of the invention include miRNA sequences, optionally end-modified miRNA sequences, to enhance entry into RISC. The miRNA sequence can be similar or identical to that of any naturally occurring miRNA (see e.g. The miRNA Registry; Griffiths-Jones S, Nuc. Acids Res., 2004). Over one thousand natural miRNAs have been identified to date and together they are thought to comprise about 1% of all predicted genes in the genome. Many natural miRNAs are clustered together in the introns of pre-mRNAs and can be identified in silico using homology-based searches (Pasquinelli et al., 2000; Lagos-Quintana et al., 2001; Lau et al., 2001; Lee and Ambros, 2001) or computer algorithms (e.g. MiRScan, MiRSeeker) that predict the capability of a candidate miRNA gene to form the stem loop structure of a pri-mRNA (Grad et al., Mol. Cell., 2003; Lim et al., Genes Dev., 2003; Lim et al., Science, 2003; Lai E C et al., Genome Bio., 2003). An online registry provides a searchable database of all published miRNA sequences (The miRNA Registry at the Sanger Institute website; Griffiths-Jones S, Nuc. Acids Res., 2004). Exemplary, natural miRNAs include lin-4, let-7, miR-10, mirR-15, miR-16, miR-168, miR-175, miR-196 and their homologs, as well as other natural miRNAs from humans and certain model organisms including Drosophila melanogaster, Caenorhabditis elegans, zebrafish, Arabidopsis thalania, Mus musculus, and Rattus norvegicus as described in International PCT Publication No. WO 03/029459.

Naturally-occurring miRNAs are expressed by endogenous genes in vivo and are processed from a hairpin or stem-loop precursor (pre-miRNA or pri-miRNAs) by Dicer or other RNAses (Lagos-Quintana et al., Science, 2001; Lau et al., Science, 2001; Lee and Ambros, Science, 2001; Lagos-Quintana et al., Curr. Biol., 2002; Mourelatos et al., Genes Dev., 2002; Reinhart et al., Science, 2002; Ambros et al., Curr. Biol., 2003; Brennecke et al., 2003; Lagos-Quintana et al., RNA, 2003; Lim et al., Genes Dev., 2003; Lim et al., Science, 2003). miRNAs can exist transiently in vivo as a double-stranded duplex, but only one strand is taken up by the RISC complex to direct gene silencing. Certain miRNAs, e.g., plant miRNAs, have perfect or near-perfect complementarity to their target mRNAs and, hence, direct cleavage of the target mRNAs. Other miRNAs have less than perfect complementarity to their target mRNAs and, hence, direct translational repression of the target mRNAs. The degree of complementarity between a miRNA and its target mRNA is believed to determine its mechanism of action. For example, perfect or near-perfect complementarity between a miRNA and its target mRNA is predictive of a cleavage mechanism (Yekta et al., Science, 2004), whereas less than perfect complementarity is predictive of a translational repression mechanism. In certain embodiments, the miRNA sequence is that of a naturally-occurring miRNA sequence, the aberrant expression or activity of which is correlated with a miRNA disorder.

### d) Dual Functional Oligonucleotide Tethers

In other embodiments, the RNA silencing agents of the present invention include dual functional oligonucleotide tethers useful for the intercellular recruitment of a miRNA. Animal cells express a range of miRNAs, noncoding RNAs of approximately 22 nucleotides which can regulate gene expression at the post transcriptional or translational level. By binding a miRNA bound to RISC and recruiting it to a target mRNA, a dual functional oligonucleotide tether can repress the expression of genes involved e.g., in the arteriosclerotic process. The use of oligonucleotide tethers offers several advantages over existing techniques to repress the expression of a particular gene. First, the methods described herein allow an endogenous molecule (often present in abundance), a miRNA, to mediate RNA silencing. Accordingly, the methods described herein obviate the need to introduce foreign molecules (e.g., siRNAs) to mediate RNA silencing. Second, the RNA-silencing agents and the linking moiety (e.g., oligonucleotides such as the 2′-O-methyl oligonucleotide), can be made stable and resistant to nuclease activity. As a result, the tethers of the present invention can be designed for direct delivery, obviating the need for indirect delivery (e.g. viral) of a precursor molecule or plasmid designed to make the desired agent within the cell. Third, tethers and their respective moieties, can be designed to conform to specific mRNA sites and specific miRNAs. The designs can be cell and gene product specific. Fourth, the methods disclosed herein leave the mRNA intact, allowing one skilled in the art to block protein synthesis in short pulses using the cell's own machinery. As a result, these methods of RNA silencing are highly regulatable.

The dual functional oligonucleotide tethers (“tethers”) of the invention are designed such that they recruit miRNAs (e.g., endogenous cellular miRNAs) to a target mRNA so as to induce the modulation of a gene of interest. In certain embodiments, the tethers have the formula T-L-μ, wherein T is an mRNA targeting moiety, L is a linking moiety, and μ is a miRNA recruiting moiety. Any one or more moiety may be double stranded. In certain embodiments, each moiety is single stranded.

Moieties within the tethers can be arranged or linked (in the 5′ to 3′ direction) as depicted in the formula T-L-μ (i.e., the 3′ end of the targeting moiety linked to the 5′ end of the linking moiety and the 3′ end of the linking moiety linked to the 5′ end of the miRNA recruiting moiety). Alternatively, the moieties can be arranged or linked in the tether as follows: μ-T-L (i.e., the 3′ end of the miRNA recruiting moiety linked to the 5′ end of the linking moiety and the 3′ end of the linking moiety linked to the 5′ end of the targeting moiety).

The mRNA targeting moiety, as described above, is capable of capturing a specific target mRNA. According to the invention, expression of the target mRNA is undesirable, and, thus, translational repression of the mRNA is desired. The mRNA targeting moiety should be of sufficient size to effectively bind the target mRNA. The length of the targeting moiety will vary greatly, depending, in part, on the length of the target mRNA and the degree of complementarity between the target mRNA and the targeting moiety. In various embodiments, the targeting moiety is less than about 200, 100, 50, 30, 25, 20, 19, 18, 17, 16, 15, 14, 13, 12, 11, 10, 9, 8, 7, 6, or 5 nucleotides in length. In a certain embodiment, the targeting moiety is about 15 to about 25 nucleotides in length.

The miRNA recruiting moiety, as described above, is capable of associating with a miRNA. According to the present application, the miRNA may be any miRNA capable of repressing the target mRNA. Mammals are reported to have over 250 endogenous miRNAs (Lagos-Quintana et al. (2002) Current Biol. 12:735-739; Lagos-Quintana et al. (2001) Science 294:858-862; and Lim et al. (2003) Science 299:1540). In various embodiments, the miRNA may be any art-recognized miRNA.

The linking moiety is any agent capable of linking the targeting moieties such that the activity of the targeting moieties is maintained. Linking moieties can be oligonucleotide moieties comprising a sufficient number of nucleotides, such that the targeting agents can sufficiently interact with their respective targets. Linking moieties have little or no sequence homology with cellular mRNA or miRNA sequences. Exemplary linking moieties include one or more 2′-O-methylnucleotides, e.g., 2′-β-methyladenosine, 2′-O-methylthymidine, 2′-O-methylguanosine or 2′-O-methyluridine.

### e) Gene Silencing Oligonucleotides

In certain exemplary embodiments, gene expression (i.e., SARS-CoV-2 gene expression) can be modulated using oligonucleotide-based compounds comprising two or more single stranded antisense oligonucleotides that are linked through their 5′-ends that allow the presence of two or more accessible 3′-ends to effectively inhibit or decrease SARS-CoV-2 gene expression. Such linked oligonucleotides are also known as Gene Silencing Oligonucleotides (GSOs). (See, e.g., U.S. Pat. No. 8,431,544 assigned to Idera Pharmaceuticals, Inc., incorporated herein by reference in its entirety for all purposes.)

The linkage at the 5′ ends of the GSOs is independent of the other oligonucleotide linkages and may be directly via 5′, 3′ or 2′hydroxyl groups, or indirectly, via a non-nucleotide linker or a nucleoside, utilizing either the 2′ or 3′ hydroxyl positions of the nucleoside. Linkages may also utilize a functionalized sugar or nucleobase of a 5′ terminal nucleotide.

GSOs can comprise two identical or different sequences conjugated at their 5′-5′ ends via a phosphodiester, phosphorothioate or non-nucleoside linker. Such compounds may comprise 15 to 27 nucleotides that are complementary to specific portions of mRNA targets of interest for antisense down regulation of a gene product. GSOs that comprise identical sequences can bind to a specific mRNA via Watson-Crick hydrogen bonding interactions and inhibit protein expression. GSOs that comprise different sequences are able to bind to two or more different regions of one or more mRNA target and inhibit protein expression. Such compounds are comprised of heteronucleotide sequences complementary to target mRNA and form stable duplex structures through Watson-Crick hydrogen bonding. Under certain conditions, GSOs containing two free 3′-ends (5′-5′-attached antisense) can be more potent inhibitors of gene expression than those containing a single free 3′-end or no free 3′-end.

In some embodiments, the non-nucleotide linker is glycerol or a glycerol homolog of the formula HO—(CH₂)ₒ—CH(OH)—(CH₂)ₚ—OH, wherein o and p independently are integers from 1 to about 6, from 1 to about 4 or from 1 to about 3. In some other embodiments, the non-nucleotide linker is a derivative of 1,3-diamino-2-hydroxypropane. Some such derivatives have the formula HO—(CH₂)m--C(O)NH—CH₂—CH(OH)—CH₂—NHC(O)—(CH₂)ₘ—OH, wherein m is an integer from 0 to about 10, from 0 to about 6, from 2 to about 6 or from 2 to about 4.

Some non-nucleotide linkers permit attachment of more than two GSO components. For example, the non-nucleotide linker glycerol has three hydroxyl groups to which GSO components may be covalently attached. Some oligonucleotide-based compounds of the invention, therefore, comprise two or more oligonucleotides linked to a nucleotide or a non-nucleotide linker. Such oligonucleotides according to the invention are referred to as being “branched.”

In certain embodiments, GSOs are at least 14 nucleotides in length. In certain exemplary embodiments, GSOs are 15 to 40 nucleotides long or 20 to 30 nucleotides in length. Thus, the component oligonucleotides of GSOs can independently be 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

These oligonucleotides can be prepared by the art recognized methods, such as phosphoramidate or H-phosphonate chemistry, which can be carried out manually or by an automated synthesizer. These oligonucleotides may also be modified in a number of ways without compromising their ability to hybridize to mRNA. Such modifications may include at least one internucleotide linkage of the oligonucleotide being an alkylphosphonate, phosphorothioate, phosphorodithioate, methylphosphonate, phosphate ester, alkylphosphonothioate, phosphoramidate, carbamate, carbonate, phosphate hydroxyl, acetamidate, carboxymethyl ester, or a combination of these and other internucleotide linkages between the 5′ end of one nucleotide and the 3′ end of another nucleotide, in which the 5′ nucleotide phosphodiester linkage has been replaced with any number of chemical groups.

### V. Modified Anti-SARS-CoV-2 RNA Silencing Agents

In certain aspects of the invention, an RNA silencing agent (or any portion thereof) of the present application, as described supra, may be modified, such that the activity of the agent is further improved. For example, the RNA silencing agents described in Section II supra, may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

### 1) Modifications to Enhance Target Discrimination

In certain embodiments, the RNA silencing agents of the present application may be substituted with a destabilizing nucleotide to enhance single nucleotide target discrimination (see U.S. application Ser. No. 11/698,689, filed Jan. 25, 2007 and U.S. Provisional Application No. 60/762,225 filed Jan. 25, 2006, both of which are incorporated herein by reference). Such a modification may be sufficient to abolish the specificity of the RNA silencing agent for a non-target mRNA (e.g. wild-type mRNA), without appreciably affecting the specificity of the RNA silencing agent for a target mRNA (e.g. gain-of-function mutant mRNA).

In certain embodiments, the RNA silencing agents of the present application are modified by the introduction of at least one universal nucleotide in the antisense strand thereof. Universal nucleotides comprise base portions that are capable of base pairing indiscriminately with any of the four conventional nucleotide bases (e.g. A, G, C, U). A universal nucleotide is contemplated because it has relatively minor effect on the stability of the RNA duplex or the duplex formed by the guide strand of the RNA silencing agent and the target mRNA. Exemplary universal nucleotides include those having an inosine base portion or an inosine analog base portion selected from the group consisting of deoxyinosine (e.g. 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In certain embodiments, the universal nucleotide is an inosine residue or a naturally occurring analog thereof.

In certain embodiments, the RNA silencing agents of the invention are modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destablilizing nucleotide and the specificity-determining nucleotide). In RNA silencing agents having two strands or strand portions (e.g. siRNAs and shRNAs), the destabilizing nucleotide may be introduced in the strand or strand portion that does not contain the specificity-determining nucleotide. In certain embodiments, the destabilizing nucleotide is introduced in the same strand or strand portion that contains the specificity-determining nucleotide.

### 2) Modifications to Enhance Efficacy and Specificity

In certain embodiments, the RNA silencing agents of the invention may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the present application or an siRNA produced from a shRNA) into RISC in favor of the sense strand, such that the antisense strand preferentially guides cleavage or translational repression of a target mRNA, and thus increasing or improving the efficiency of target cleavage and silencing. In certain embodiments, the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the present application may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the invention may be enhanced such that there is at least one mismatched base pair between the 5′ end of the first or antisense strand and the 3′ end of the sense strand portion. In certain embodiments, the mismatched base pair is selected from the group consisting of G:A, C:A, C:U, G:G, A:A, C:C and U:U. In another embodiment, the asymmetry of an RNA silencing agent of the invention may be enhanced such that there is at least one wobble base pair, e.g., G:U, between the 5′ end of the first or antisense strand and the 3′ end of the sense strand portion. In another embodiment, the asymmetry of an RNA silencing agent of the invention may be enhanced such that there is at least one base pair comprising a rare nucleotide, e.g., inosine (I). In certain embodiments, the base pair is selected from the group consisting of an I:A, I:U and I:C. In yet another embodiment, the asymmetry of an RNA silencing agent of the invention may be enhanced such that there is at least one base pair comprising a modified nucleotide. In certain embodiments, the modified nucleotide is selected from the group consisting of 2-amino-G, 2-amino-A, 2,6-diamino-G, and 2,6-diamino-A.

3) RNA Silencing Agents with Enhanced Stability

The RNA silencing agents of the present application can be modified to improve stability in serum or in growth medium for cell cultures. In order to enhance the stability, the 3′-residues may be stabilized against degradation, e.g., they may be selected such that they consist of purine nucleotides, such as adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a one aspect, the present application features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In one aspect, the present application features RNA silencing agents that are at least 80% chemically modified. In certain embodiments, the RNA silencing agents may be fully chemically modified, i.e., 100% of the nucleotides are chemically modified. In another aspect, the present application features RNA silencing agents comprising 2′-OH ribose groups that are at least 80% chemically modified. In certain embodiments, the RNA silencing agents comprise 2′-OH ribose groups that are about 80%, 85%, 90%, 95%, or 100% chemically modified.

In certain embodiments, the RNA silencing agents may contain at least one modified nucleotide analogue. The nucleotide analogues may be located at positions where the target-specific silencing activity, e.g., the RNAi mediating activity or translational repression activity is not substantially affected, e.g., in a region at the 5′-end and/or the 3′-end of the siRNA molecule. Moreover, the ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphorothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In certain embodiments, the modifications are 2′-fluoro, 2′-amino and/or 2′-thio modifications. Modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a certain embodiment, the 2′-fluoro ribonucleotides are every uridine and cytidine. Additional exemplary modifications include 5-bromo-uridine, 5-iodo-uridine, 5-methyl-cytidine, ribothymidine, 2-aminopurine, 2′-amino-butyryl-pyrene-uridine, 5-fluoro-cytidine, and 5-fluoro-uridine. 2′-deoxy-nucleotides and 2′-O-Me nucleotides can also be used within modified RNA-silencing agents of the instant invention. Additional modified residues include, deoxy-abasic, inosine, N3-methyl-uridine, N6,N6-dimethyl-adenosine, pseudouridine, purine ribonucleoside and ribavirin. In a certain embodiment, the 2′ moiety is a methyl group such that the linking moiety is a 2′-O-methyl oligonucleotide.

In a certain embodiment, the RNA silencing agent of the present application comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-O,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the present application comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone, which is highly resistant to nuclease digestion and imparts improved binding specificity to the molecule (Nielsen, et al., Science, (2001), 254: 1497-1500).

Also contemplated are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the present application includes RNA silencing agents having two complementary strands of nucleic acid, wherein the two strands are crosslinked. The present application also includes RNA silencing agents which are conjugated or unconjugated (e.g., at its 3′ terminus) to another moiety (e.g. a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Other exemplary modifications include: (a) 2′ modification, e.g., provision of a 2′ OMe moiety on a U in a sense or antisense strand, but especially on a sense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an O with an S, in the phosphate backbone, e.g., the provision of a phosphorothioate modification, on the U or the A or both, especially on an antisense strand; e.g., with the replacement of a O with an S; (c) replacement of the U with a C5 amino linker; (d) replacement of an A with a G (sequence changes can be located on the sense strand and not the antisense strand in certain embodiments); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ O Me moiety and modification of the backbone, e.g., with the replacement of a O with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### Heavily Modified RNA Silencing Agents

In certain embodiments, the RNA silencing agent comprises at least 80% chemically modified nucleotides. In certain embodiments, the RNA silencing agent is fully chemically modified, i.e., 100% of the nucleotides are chemically modified.

In certain embodiments, the RNA silencing agent is 2′-O-methyl rich, i.e., comprises greater than 50% 2′-O-methyl content. In certain embodiments, the RNA silencing agent comprises at least about 55%, 60%, 65%, 70%, 75%, 80%, 85%, 90%, 95%, or 100% 2′-O-methyl nucleotide content. In certain embodiments, the RNA silencing agent comprises at least about 70% 2′-O-methyl nucleotide modifications. In certain embodiments, the RNA silencing agent comprises between about 70% and about 90% 2′-O-methyl nucleotide modifications. In certain embodiments, the RNA silencing agent is a dsRNA comprising an antisense strand and sense strand. In certain embodiments, the antisense strand comprises at least about 70% 2′-O-methyl nucleotide modifications. In certain embodiments, the antisense strand comprises between about 70% and about 90% 2′-O-methyl nucleotide modifications. In certain embodiments, the sense strand comprises at least about 70% 2′-O-methyl nucleotide modifications. In certain embodiments, the sense strand comprises between about 70% and about 90% 2′-O-methyl nucleotide modifications. In certain embodiments, the sense strand comprises between 100% 2′-O-methyl nucleotide modifications.

2′-O-methyl rich RNA silencing agents and specific chemical modification patterns are further described in U.S. Ser. No. 16/550,076 (filed Aug. 23, 2019) and U.S. Ser. No. 62/891,185 (filed Aug. 23, 2019), each of which is incorporated herein by reference.

### Internucleotide Linkage Modifications

In certain embodiments, at least one internucleotide linkage, intersubunit linkage, or nucleotide backbone is modified in the RNA silencing agent. In certain embodiments, all of the internucleotide linkages in the RNA silencing agent are modified. In certain embodiments, the modified internucleotide linkage comprises a phosphorothioate internucleotide linkage. In certain embodiments, the RNA silencing agent comprise 1, 2, 3, 4, 5, 6, 7, 8, 9, 10, 11, 12, 13, 14, 15, 16, 17, 18, 19, 20, 21, 22, 23, 24, or 25 phosphorothioate internucleotide linkages. In certain embodiments, the RNA silencing agent comprises 4-16 phosphorothioate internucleotide linkages. In certain embodiments, the RNA silencing agent comprises 8-13 phosphorothioate internucleotide linkages. In certain embodiments, the RNA silencing agent is a dsRNA comprising an antisense strand and a sense strand, each comprising a 5′ end and a 3′ end. In certain embodiments, the nucleotides at positions 1 and 2 from the 5′ end of sense strand are connected to adjacent ribonucleotides via phosphorothioate internucleotide linkages. In certain embodiments, the nucleotides at positions 1 and 2 from the 3′ end of sense strand are connected to adjacent ribonucleotides via phosphorothioate internucleotide linkages. In certain embodiments, the nucleotides at positions 1 and 2 from the 5′ end of antisense strand are connected to adjacent ribonucleotides via phosphorothioate internucleotide linkages. In certain embodiments, the nucleotides at positions 1-2 to 1-8 from the 3′ end of antisense strand are connected to adjacent ribonucleotides via phosphorothioate internucleotide linkages. In certain embodiments, the nucleotides at positions 1-2, 1-3, 1-4, 1-5, 1-6, 1-7, or 1-8 from the 3′ end of antisense strand are connected to adjacent ribonucleotides via phosphorothioate internucleotide linkages. In certain embodiments, the nucleotides at positions 1-2 to 1-7 from the 3′ end of antisense strand are connected to adjacent ribonucleotides via phosphorothioate internucleotide linkages.

In one aspect, the disclosure provides a modified oligonucleotide, said oligonucleotide having a 5′ end, a 3′ end, that is complementary to a target, wherein the oligonucleotide comprises a sense and antisense strand, and at least one modified intersubunit linkage of Formula (I):

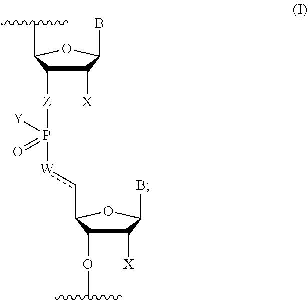

wherein:
   B is a base pairing moiety;
   W is selected from the group consisting of O, OCH₂, OCH, CH₂, and CH;
   X is selected from the group consisting of halo, hydroxy, and C₁₋₆ alkoxy;
   Y is selected from the group consisting of O⁻, OH, OR, NH⁻, NH₂, S⁻, and SH;
   Z is selected from the group consisting of O and CH₂;
   R is a protecting group; and
   [symbol] is an optional double bond.

In an embodiment of Formula (I), when W is CH, [symbol] is a double bond.

In an embodiment of Formula (I), when W selected from the group consisting of O, OCH₂, OCH, CH₂, [symbol] is a single bond.

In an embodiment of Formula (I), when Y is O⁻, either Z or W is not O.

In an embodiment of Formula (I), Z is CH₂ and W is CH₂. In another embodiment, the modified intersubunit linkage of Formula (I) is a modified intersubunit linkage of Formula (II):

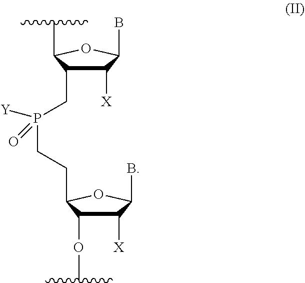

In an embodiment of Formula (I), Z is CH₂ and W is O. In another embodiment, wherein the modified intersubunit linkage of Formula (I) is a modified intersubunit linkage of Formula (III):

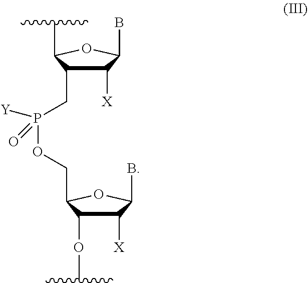

In an embodiment of Formula (I), Z is O and W is CH₂. In another embodiment, the modified intersubunit linkage of Formula (I) is a modified intersubunit linkage of Formula (IV):

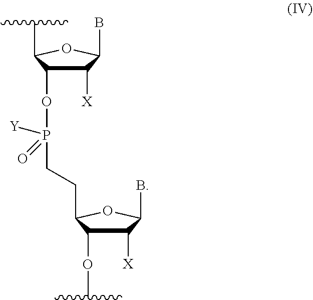

In an embodiment of Formula (I), Z is O and W is CH. In another embodiment, the modified intersubunit linkage of Formula (I) is a modified intersubunit linkage of Formula V:

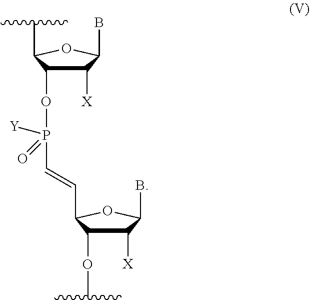

In an embodiment of Formula (I), Z is O and W is OCH₂. In another embodiment, the modified intersubunit linkage of Formula (I) is a modified intersubunit linkage of Formula VI:

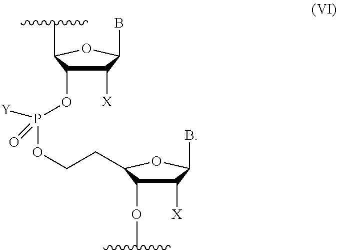

In an embodiment of Formula (I), Z is CH₂ and W is CH. In another embodiment, the modified intersubunit linkage of Formula (I) is a modified intersubunit linkage of Formula VII:

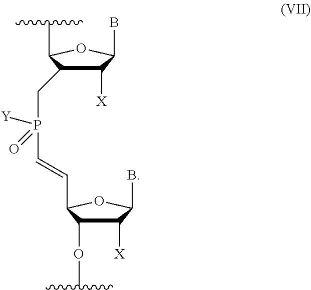

In an embodiment of Formula (I), the base pairing moiety B is selected from the group consisting of adenine, guanine, cytosine, and uracil.

In an embodiment, the modified oligonucleotide is incorporated into siRNA, said modified siRNA having a 5′ end, a 3′ end, that is complementary to a target, wherein the siRNA comprises a sense and antisense strand, and at least one modified intersubunit linkage of any one or more of Formula (I), Formula (II), Formula (III), Formula (IV), Formula (V), Formula (VI), or Formula (VII).

In an embodiment, the modified oligonucleotide is incorporated into siRNA, said modified siRNA having a 5′ end, a 3′ end, that is complementary to a target and comprises a sense and antisense strand, wherein the siRNA comprises at least one modified intersubunit linkage is of Formula VIII:

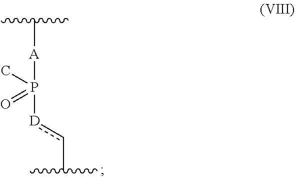

wherein:
   D is selected from the group consisting of O, OCH₂, OCH, CH₂, and CH;
   C is selected from the group consisting of O⁻, OH, OR¹, NH⁻, NH₂, S⁻, and SH;
   A is selected from the group consisting of O and CH₂;
R¹ is a protecting group;
   [symbol] is an optional double bond; and
   the intersubunit is bridging two optionally modified nucleosides.

In an embodiment, when C is O—, either A or D is not O.

In an embodiment, D is CH₂. In another embodiment, the modified intersubunit linkage of Formula VIII is a modified intersubunit linkage of Formula (IX):

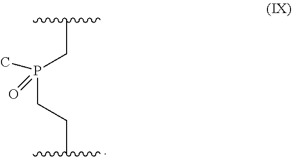 [SMILES: *CCP(C)(=O)C*]

In an embodiment, D is O. In another embodiment, the modified intersubunit linkage of Formula VIII is a modified intersubunit linkage of Formula (X):

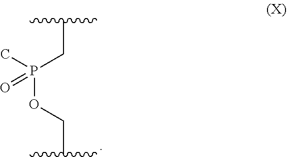 [SMILES: *COP(C)(=O)C*]

In an embodiment, D is CH₂. In another embodiment, the modified intersubunit linkage of Formula (VIII) is a modified intersubunit linkage of Formula (XI):

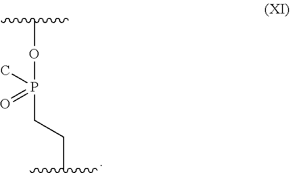 [SMILES: *CCP(C)(=O)O*]

In an embodiment, D is CH. In another embodiment, the modified intersubunit linkage of Formula VIII is a modified intersubunit linkage of Formula (XII):

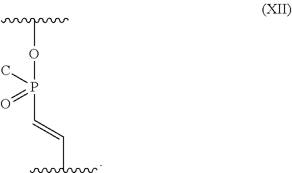 [SMILES: */C=C/P(C)(=O)O*]

In another embodiment, the modified intersubunit linkage of Formula (VII) is a modified intersubunit linkage of Formula (XIV):

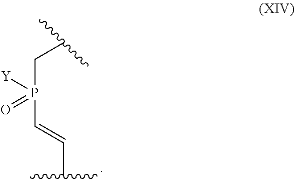

In an embodiment, D is OCH₂. In another embodiment, the modified intersubunit linkage of Formula (VII) is a modified intersubunit linkage of Formula (XIII):

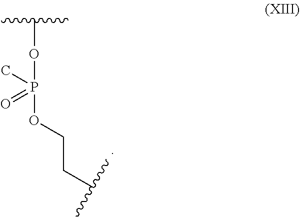 [SMILES: *CCOP(C)(=O)O*]

In another embodiment, the modified intersubunit linkage of Formula (VII) is a modified intersubunit linkage of Formula (XXa):

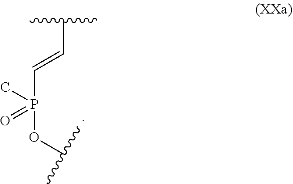 [SMILES: */C=C/P(C)(=O)O*]

In an embodiment of the modified siRNA linkage, each optionally modified nucleoside is independently, at each occurrence, selected from the group consisting of adenosine, guanosine, cytidine, and uridine.

In certain exemplary embodiments of Formula (I), W is O. In another embodiment, W is CH₂. In yet another embodiment, W is CH.

In certain exemplary embodiments of Formula (I), X is OH. In another embodiment, X is OCH₃. In yet another embodiment, X is halo.

In a certain embodiment of Formula (I), the modified siRNA does not comprise a 2′-fluoro substituent.

In an embodiment of Formula (I), Y is O. In another embodiment, Y is OH. In yet another embodiment, Y is OR. In still another embodiment, Y is NH⁻. In an embodiment, Y is NH₂. In another embodiment, Y is S⁻. In yet another embodiment, Y is SH.

In an embodiment of Formula (I), Z is O. In another embodiment, Z is CH₂.

In an embodiment, the modified intersubunit linkage is inserted on position 1-2 of the antisense strand. In another embodiment, the modified intersubunit linkage is inserted on position 6-7 of the antisense strand. In yet another embodiment, the modified intersubunit linkage is inserted on position 10-11 of the antisense strand. In still another embodiment, the modified intersubunit linkage is inserted on position 19-20 of the antisense strand. In an embodiment, the modified intersubunit linkage is inserted on positions 5-6 and 18-19 of the antisense strand.

In an exemplary embodiment of the modified siRNA linkage of Formula (VIII), C is O⁻. In another embodiment, C is OH. In yet another embodiment, C is OR¹. In still another embodiment, C is NH⁻. In an embodiment, C is NH₂. In another embodiment, C is S⁻. In yet another embodiment, C is SH.

In an exemplary embodiment of the modified siRNA linkage of Formula (VIII), A is O. In another embodiment, A is CH₂. In yet another embodiment, C is OR¹. In still another embodiment, C is NH⁻. In an embodiment, C is NH₂. In another embodiment, C is S⁻. In yet another embodiment, C is SH.

In a certain embodiment of the modified siRNA linkage of Formula (VIII), the optionally modified nucleoside is adenosine. In another embodiment of the modified siRNA linkage of Formula (VIII), the optionally modified nucleoside is guanosine. In another embodiment of the modified siRNA linkage of Formula (VIII), the optionally modified nucleoside is cytidine. In another embodiment of the modified siRNA linkage of Formula (VIII), the optionally modified nucleoside is uridine.

In an embodiment of the modified siRNA linkage, wherein the linkage is inserted on position 1-2 of the antisense strand. In another embodiment, the linkage is inserted on position 6-7 of the antisense strand. In yet another embodiment, the linkage is inserted on position 10-11 of the antisense strand. In still another embodiment, the linkage is inserted on position 19-20 of the antisense strand. In an embodiment, the linkage is inserted on positions 5-6 and 18-19 of the antisense strand.

In certain embodiments of Formula (I), the base pairing moiety B is adenine. In certain embodiments of Formula (I), the base pairing moiety B is guanine. In certain embodiments of Formula (I), the base pairing moiety B is cytosine. In certain embodiments of Formula (I), the base pairing moiety B is uracil.

In an embodiment of Formula (I), W is O. In an embodiment of Formula (I), W is CH₂. In an embodiment of Formula (I), W is CH.

In an embodiment of Formula (I), X is OH. In an embodiment of Formula (I), X is OCH₃. In an embodiment of Formula (I), X is halo.

In an exemplary embodiment of Formula (I), the modified oligonucleotide does not comprise a 2′-fluoro substituent.

In an embodiment of Formula (I), Y is O⁻. In an embodiment of Formula (I), Y is OH. In an embodiment of Formula (I), Y is OR. In an embodiment of Formula (I), Y is NH⁻. In an embodiment of Formula (I), Y is NH₂. In an embodiment of Formula (I), Y is S⁻. In an embodiment of Formula (I), Y is SH.

In an embodiment of Formula (I), Z is O. In an embodiment of Formula (I), Z is CH₂.

In an embodiment of the Formula (I), the linkage is inserted on position 1-2 of the antisense strand. In another embodiment of Formula (I), the linkage is inserted on position 6-7 of the antisense strand. In yet another embodiment of Formula (I), the linkage is inserted on position 10-11 of the antisense strand. In still another embodiment of Formula (I), the linkage is inserted on position 19-20 of the antisense strand. In an embodiment of Formula (I), the linkage is inserted on positions 5-6 and 18-19 of the antisense strand.

Modified intersubunit linkages are further described in WO20200198509 and PCT/US2021/024425, each of which is incorporated herein by reference.

### 4) Conjugated Functional Moieties

In other embodiments, RNA silencing agents may be modified with one or more functional moieties. A functional moiety is a molecule that confers one or more additional activities to the RNA silencing agent. In certain embodiments, the functional moieties enhance cellular uptake by target cells (e.g., lung cells). Thus, the invention includes RNA silencing agents which are conjugated or unconjugated (e.g., at its 5′ and/or 3′ terminus) to another moiety (e.g. a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a certain embodiment, the functional moiety is a hydrophobic moiety. In a certain embodiment, the hydrophobic moiety is selected from the group consisting of fatty acids, steroids, secosteroids, lipids, gangliosides and nucleoside analogs, endocannabinoids, and vitamins. In a certain embodiment, the steroid selected from the group consisting of cholesterol and Lithocholic acid (LCA). In a certain embodiment, the fatty acid selected from the group consisting of Eicosapentaenoic acid (EPA), Docosahexaenoic acid (DHA) and Docosanoic acid (DCA). In a certain embodiment, the vitamin selected from the group consisting of choline, vitamin A, vitamin E, and derivatives or metabolites thereof. In a certain embodiment, the vitamin is selected from the group consisting of retinoic acid and alpha-tocopheryl succinate.

In a certain embodiment, an RNA silencing agent of invention is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the Y end of the sense strand. In certain embodiments, the lipophilic moiety is selected from the group consisting of cholesterol, vitamin E, vitamin K, vitamin A, folic acid, a cationic dye (e.g., Cy3). In an exemplary embodiment, the lipophilic moiety is cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In certain embodiments, the functional moieties may comprise one or more ligands tethered to an RNA silencing agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These can be located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., O-phenanthroline), a polyamine, a tripeptide (e.g., lys-tyr-lys tripeptide), or a metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to a RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine, has an increased affinity for the HIV Rev-response element (RRE). In some embodiments, the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, either directly or indirectly, via an intervening tether, to a ligand-conjugated carrier. In certain embodiments, the coupling is through a covalent bond. In certain embodiments, the ligand is attached to the carrier via an intervening tether. In certain embodiments, a ligand alters the distribution, targeting or lifetime of an RNA silencing agent into which it is incorporated. In certain embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine (GalNAc) or derivatives thereof, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycosides, artificial endonucleases (e.g. EDTA), lipophilic molecules, e.g, cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₈, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, 03-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu³⁺ complexes of tetraazamacrocycles), dinitrophenyl, HRP or AP. In certain embodiments, the ligand is GalNAc or a derivative thereof.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-kB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNFQ), interleukin-1 beta, or gamma interferon. In one aspect, the ligand is a lipid or lipid-based molecule. Such a lipid or lipid-based molecule can bind a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a certain embodiment, the lipid based ligand binds HSA. A lipid-based ligand can bind HSA with a sufficient affinity such that the conjugate will be distributed to a non-kidney tissue. However, it is contemplated that the affinity not be so strong that the HSA-ligand binding cannot be reversed. In another embodiment, the lipid based ligand binds HSA weakly or not at all, such that the conjugate will be distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These can be useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent can be an alpha-helical agent, which may have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

In certain embodiments, the functional moiety is linked to the 5′ end and/or 3′ end of the RNA silencing agent of the disclosure. In certain embodiments, the functional moiety is linked to the 5′ end and/or 3′ end of an antisense strand of the RNA silencing agent of the disclosure. In certain embodiments, the functional moiety is linked to the 5′ end and/or 3′ end of a sense strand of the RNA silencing agent of the disclosure. In certain embodiments, the functional moiety is linked to the 3′ end of a sense strand of the RNA silencing agent of the disclosure.

In certain embodiments, the functional moiety is linked to the RNA silencing agent by a linker. In certain embodiments, the functional moiety is linked to the antisense strand and/or sense strand by a linker. In certain embodiments, the functional moiety is linked to the 3′ end of a sense strand by a linker. In certain embodiments, the linker comprises a divalent or trivalent linker. In certain embodiments, the linker comprises an ethylene glycol chain, an alkyl chain, a peptide, RNA, DNA, a phosphodiester, a phosphorothioate, a phosphoramidate, an amide, a carbamate, or a combination thereof. In certain embodiments, the divalent or trivalent linker is selected from:

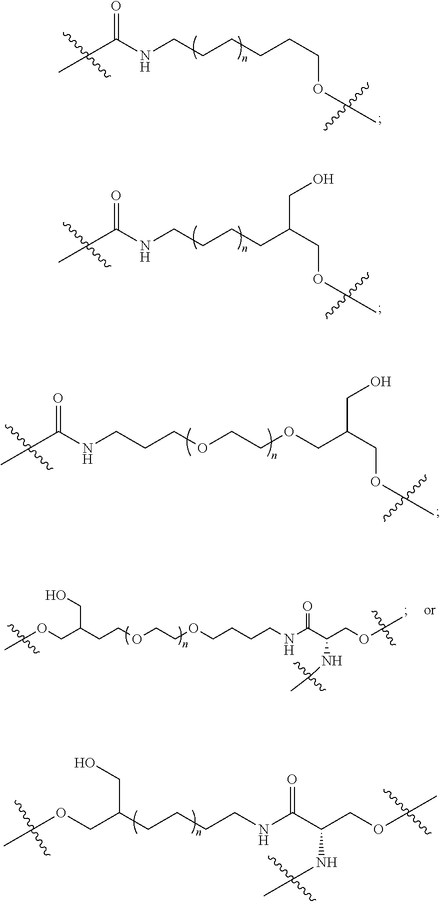

wherein n is 1, 2, 3, 4, or 5.

In certain embodiments, the linker further comprises a phosphodiester or phosphodiester derivative. In certain embodiments, the phosphodiester or phosphodiester derivative is selected from the group consisting of:

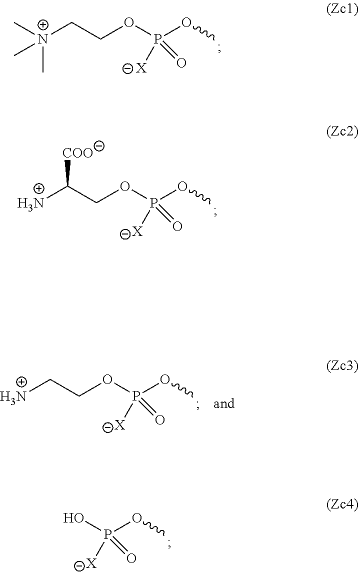

wherein X is O, S or BH₃.

The various functional moieties of the disclosure and means to conjugate them to RNA silencing agents are described in further detail in WO2017/030973A1 and WO2018/031933A2, incorporated herein by reference.

### VI. Branched Oligonucleotides

Two or more RNA silencing agents as disclosed supra, for example oligonucleotide constructs such as anti-SARS-CoV-2 siRNAs, may be connected to one another by one or more moieties independently selected from a linker, a spacer and a branching point, to form a branched oligonucleotide RNA silencing agent. In certain embodiments, the branched oligonucleotide RNA silencing agent consists of two siRNAs to form a di-branched siRNA (“di-siRNA”) scaffolding for delivering two siRNAs. In representative embodiments, the nucleic acids of the branched oligonucleotide each comprise an antisense strand (or portions thereof), wherein the antisense strand has sufficient complementarity to a target mRNA (e.g., SARS-CoV-2 mRNA) to mediate an RNA-mediated silencing mechanism (e.g. RNAi).

In exemplary embodiments, the branched oligonucleotides may have two to eight RNA silencing agents attached through a linker. The linker may be hydrophobic. In an embodiment, branched oligonucleotides of the present application have two to three oligonucleotides. In an embodiment, the oligonucleotides independently have substantial chemical stabilization (e.g., at least 40% of the constituent bases are chemically-modified). In an exemplary embodiment, the oligonucleotides have full chemical stabilization (i.e., all the constituent bases are chemically-modified). In some embodiments, branched oligonucleotides comprise one or more single-stranded phosphorothioated tails, each independently having two to twenty nucleotides. In a non-limiting embodiment, each single-stranded tail has two to ten nucleotides.

In certain embodiments, branched oligonucleotides are characterized by three properties: (1) a branched structure, (2) full metabolic stabilization, and (3) the presence of a single-stranded tail comprising phosphorothioate linkers. In certain embodiments, branched oligonucleotides have 2 or 3 branches. It is believed that the increased overall size of the branched structures promotes increased uptake. Also, without being bound by a particular theory of activity, multiple adjacent branches (e.g., 2 or 3) are believed to allow each branch to act cooperatively and thus dramatically enhance rates of internalization, trafficking and release.

Branched oligonucleotides are provided in various structurally diverse embodiments. In some embodiments nucleic acids attached at the branching points are single stranded or double stranded and consist of miRNA inhibitors, gapmers, mixmers, SSOs, PMOs, or PNAs. These single strands can be attached at their 3′ or 5′ end. Combinations of siRNA and single stranded oligonucleotides could also be used for dual function. In another embodiment, short nucleic acids complementary to the gapmers, mixmers, miRNA inhibitors, SSOs, PMOs, and PNAs are used to carry these active single-stranded nucleic acids and enhance distribution and cellular internalization. The short duplex region has a low melting temperature (Tm ˜37° C.) for fast dissociation upon internalization of the branched structure into the cell.

The Di-siRNA branched oligonucleotides may comprise chemically diverse conjugates, such as the functional moieties described above. Conjugated bioactive ligands may be used to enhance cellular specificity and to promote membrane association, internalization, and serum protein binding. Examples of bioactive moieties to be used for conjugation include DHA, GalNAc, and cholesterol. These moieties can be attached to Di-siRNA either through the connecting linker or spacer, or added via an additional linker or spacer attached to another free siRNA end.

Branched oligonucleotides comprise a variety of therapeutic nucleic acids, including siRNAs, ASOs, miRNAs, miRNA inhibitors, splice switching, PMOs, PNAs. In some embodiments, branched oligonucleotides further comprise conjugated hydrophobic moieties and exhibit unprecedented silencing and efficacy in vitro and in vivo.

### Linkers

In an embodiment of the branched oligonucleotide, each linker is independently selected from an ethylene glycol chain, an alkyl chain, a peptide, RNA, DNA, a phosphate, a phosphonate, a phosphoramidate, an ester, an amide, a triazole, and combinations thereof; wherein any carbon or oxygen atom of the linker is optionally replaced with a nitrogen atom, bears a hydroxyl substituent, or bears an oxo substituent. In one embodiment, each linker is an ethylene glycol chain. In another embodiment, each linker is an alkyl chain. In another embodiment, each linker is a peptide. In another embodiment, each linker is RNA. In another embodiment, each linker is DNA. In another embodiment, each linker is a phosphate. In another embodiment, each linker is a phosphonate. In another embodiment, each linker is a phosphoramidate. In another embodiment, each linker is an ester. In another embodiment, each linker is an amide. In another embodiment, each linker is a triazole.

### VII. Compound of Formula (I)

In another aspect, provided herein is a branched oligonucleotide compound of formula (I):

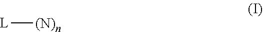

wherein L is selected from an ethylene glycol chain, an alkyl chain, a peptide, RNA, DNA, a phosphate, a phosphonate, a phosphoramidate, an ester, an amide, a triazole, and combinations thereof, wherein formula (I) optionally further comprises one or more branch point B, and one or more spacer S; wherein B is independently for each occurrence a polyvalent organic species or derivative thereof; S is independently for each occurrence selected from an ethylene glycol chain, an alkyl chain, a peptide, RNA, DNA, a phosphate, a phosphonate, a phosphoramidate, an ester, an amide, a triazole, and combinations thereof.

Moiety N is an RNA duplex comprising a sense strand and an antisense strand; and n is 2, 3, 4, 5, 6, 7 or 8. In an embodiment, the antisense strand of N comprises a sequence substantially complementary to a SARS-CoV-2 nucleic acid sequence of any one of SEQ ID NOs: 1-10, as recited in Table 4 and Table 5.

In an embodiment, the antisense strand of N comprises a sequence substantially complementary to a SARS-CoV-2 nucleic acid sequence of any one of the 45-nucleotide target gene regions recited in Table 6A.

The sense strand and antisense strand may each independently comprise one or more chemical modifications.

In an embodiment, the compound of formula (I) has a structure selected from formulas (I-1)-(I-9) of Table 1.

| | |
|---|---|
| 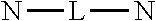 | (I-1) |
| 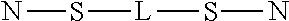 | (I-2) |
| 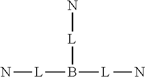 | (I-3) |
| 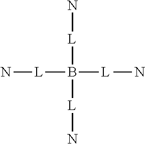 | (I-4) |
| 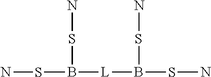 | (I-5) |
| 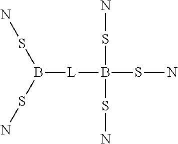 | (I-6) |
| 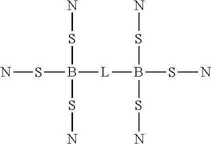 | (I-7) |
| 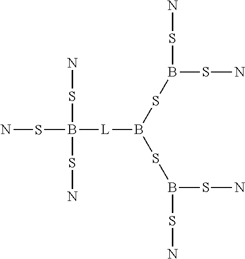 | (I-8) |
| 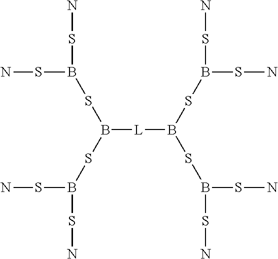 | (I-9) |

In one embodiment, the compound of formula (I) is formula (I-1). In another embodiment, the compound of formula (I) is formula (I-2). In another embodiment, the compound of formula (I) is formula (I-3). In another embodiment, the compound of formula (I) is formula (I-4). In another embodiment, the compound of formula (I) is formula (I-5). In another embodiment, the compound of formula (I) is formula (I-6). In another embodiment, the compound of formula (I) is formula (I-7). In another embodiment, the compound of formula (I) is formula (I-8). In another embodiment, the compound of formula (I) is formula (I-9).

In an embodiment of the compound of formula (I), each linker is independently selected from an ethylene glycol chain, an alkyl chain, a peptide, RNA, DNA, a phosphate, a phosphonate, a phosphoramidate, an ester, an amide, a triazole, and combinations thereof; wherein any carbon or oxygen atom of the linker is optionally replaced with a nitrogen atom, bears a hydroxyl substituent, or bears an oxo substituent. In one embodiment of the compound of formula (I), each linker is an ethylene glycol chain. In another embodiment, each linker is an alkyl chain. In another embodiment of the compound of formula (I), each linker is a peptide. In another embodiment of the compound of formula (I), each linker is RNA. In another embodiment of the compound of formula (I), each linker is DNA. In another embodiment of the compound of formula (I), each linker is a phosphate. In another embodiment, each linker is a phosphonate. In another embodiment of the compound of formula (I), each linker is a phosphoramidate. In another embodiment of the compound of formula (I), each linker is an ester. In another embodiment of the compound of formula (I), each linker is an amide. In another embodiment of the compound of formula (I), each linker is a triazole.

In one embodiment of the compound of formula (I), B is a polyvalent organic species. In another embodiment of the compound of formula (I), B is a derivative of a polyvalent organic species. In one embodiment of the compound of formula (I), B is a triol or tetrol derivative. In another embodiment, B is a tri- or tetra-carboxylic acid derivative. In another embodiment, B is an amine derivative. In another embodiment, B is a tri- or tetra-amine derivative. In another embodiment, B is an amino acid derivative. In another embodiment of the compound of formula (I), B is selected from the formulas of:

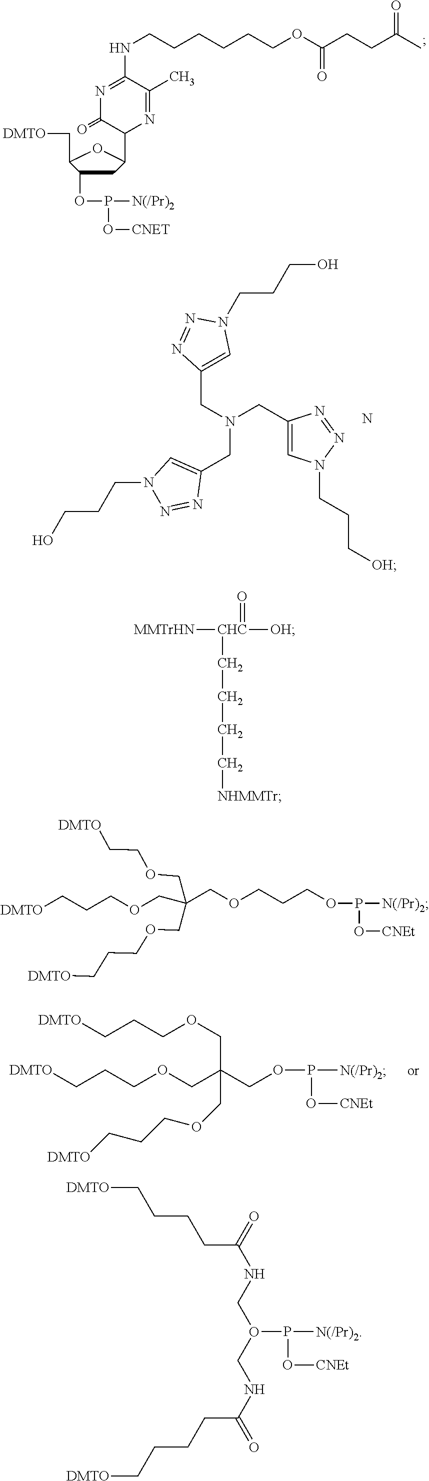

Polyvalent organic species are moieties comprising carbon and three or more valencies (i.e., points of attachment with moieties such as S, L or N, as defined above). Non-limiting examples of polyvalent organic species include triols (e.g., glycerol, phloroglucinol, and the like), tetrols (e.g., ribose, pentaerythritol, 1,2,3,5-tetrahydroxybenzene, and the like), tri-carboxylic acids (e.g., citric acid, 1,3,5-cyclohexanetricarboxylic acid, trimesic acid, and the like), tetra-carboxylic acids (e.g., ethylenediaminetetraacetic acid, pyromellitic acid, and the like), tertiary amines (e.g., tripropargylamine, triethanolamine, and the like), triamines (e.g., diethylenetriamine and the like), tetramines, and species comprising a combination of hydroxyl, thiol, amino, and/or carboxyl moieties (e.g., amino acids such as lysine, serne, cysteine, and the like).

In an embodiment of the compound of formula (I), each nucleic acid comprises one or more chemically-modified nucleotides. In an embodiment of the compound of formula (I), each nucleic acid consists of chemically-modified nucleotides. In certain embodiments of the compound of formula (I), >95%, >90%, >85%, >80%, >75%, >70%, >65%, >60%, >55% or >50% of each nucleic acid comprises chemically-modified nucleotides.

In an embodiment, each antisense strand independently comprises a 5′ terminal group R selected from the groups of Table 2.

TABLE 2

| | |
|---|---|
| 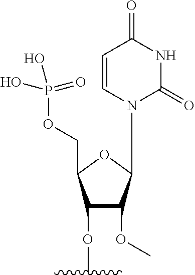 | R¹ |
| 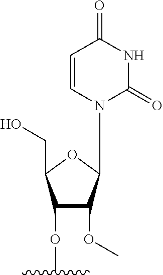 | R² |
| 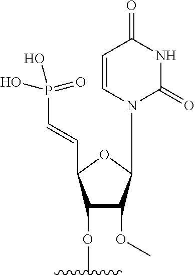 | R³ |
| 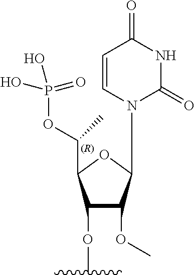 | R⁴ |
| 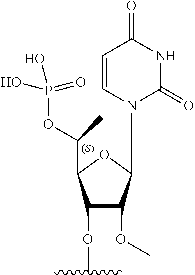 | R⁵ |
| 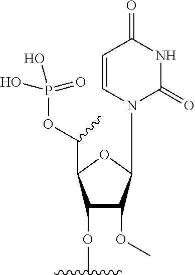 | R⁶ |
| 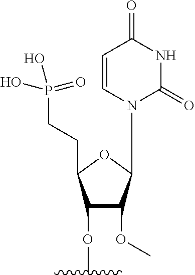 | R⁷ |
| 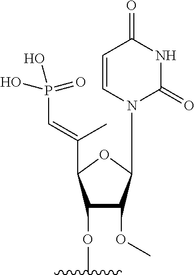 | R⁸ |

In one embodiment, R is R₁. In another embodiment, R is R₂. In another embodiment, R is R₃. In another embodiment, R is R₄. In another embodiment, R is R₅. In another embodiment, R is R₆. In another embodiment, R is R₇. In another embodiment, R is R₈.

### Structure of Formula (II)

In an embodiment, the compound of formula (I) has the structure of formula (II):

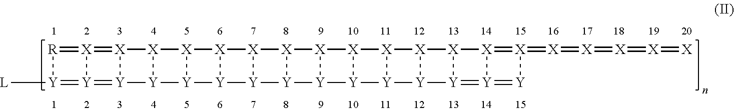

wherein X, for each occurrence, independently, is selected from adenosine, guanosine, uridine, cytidine, and chemically-modified derivatives thereof; Y, for each occurrence, independently, is selected from adenosine, guanosine, uridine, cytidine, and chemically-modified derivatives thereof; - represents a phosphodiester internucleoside linkage; =represents a phosphorothioate internucleoside linkage; and --- represents, individually for each occurrence, a base-pairing interaction or a mismatch.

In certain embodiments, the structure of formula (II) does not contain mismatches. In one embodiment, the structure of formula (II) contains 1 mismatch. In another embodiment, the compound of formula (II) contains 2 mismatches. In another embodiment, the compound of formula (II) contains 3 mismatches. In another embodiment, the compound of formula (II) contains 4 mismatches. In an embodiment, each nucleic acid consists of chemically-modified nucleotides.

In certain embodiments, >95%, >90%, >85%, >80%, >75%, >70%, >65%, >60%, >55% or >50% of X's of the structure of formula (II) are chemically-modified nucleotides. In other embodiments, >95%, >90%, >85%, >80%, >75%, >70%, >65%, >60%, >55% or >50% of X's of the structure of formula (II) are chemically-modified nucleotides.

### Structure of Formula (III)

In an embodiment, the compound of formula (I) has the structure of formula (III):

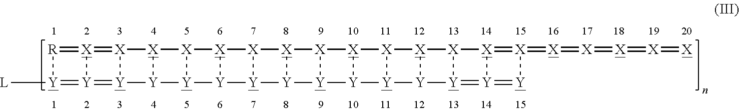

wherein X, for each occurrence, independently, is a nucleotide comprising a 2′-deoxy-2′-fluoro modification; X, for each occurrence, independently, is a nucleotide comprising a 2′-O-methyl modification; Y, for each occurrence, independently, is a nucleotide comprising a 2′-deoxy-2′-fluoro modification; and Y, for each occurrence, independently, is a nucleotide comprising a 2′-O-methyl modification.

In an embodiment, X is chosen from the group consisting of 2′-deoxy-2′-fluoro modified adenosine, guanosine, uridine or cytidine. In an embodiment, X is chosen from the group consisting of 2′-O-methyl modified adenosine, guanosine, uridine or cytidine. In an embodiment, Y is chosen from the group consisting of 2′-deoxy-2′-fluoro modified adenosine, guanosine, uridine or cytidine. In an embodiment, Y is chosen from the group consisting of 2′-O-methyl modified adenosine, guanosine, uridine or cytidine.

In certain embodiments, the structure of formula (III) does not contain mismatches. In one embodiment, the structure of formula (III) contains 1 mismatch. In another embodiment, the compound of formula (III) contains 2 mismatches. In another embodiment, the compound of formula (III) contains 3 mismatches. In another embodiment, the compound of formula (III) contains 4 mismatches.

### Structure of Formula (IV)

In an embodiment, the compound of formula (I) has the structure of formula (IV):

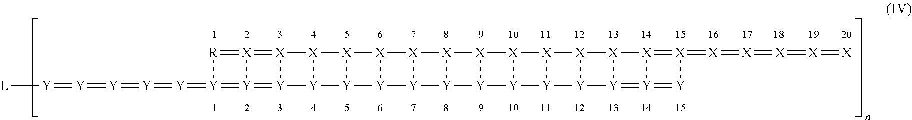

wherein X, for each occurrence, independently, is selected from adenosine, guanosine, uridine, cytidine, and chemically-modified derivatives thereof; Y, for each occurrence, independently, is selected from adenosine, guanosine, uridine, cytidine, and chemically-modified derivatives thereof; - represents a phosphodiester internucleoside linkage; =represents a phosphorothioate internucleoside linkage; and --- represents, individually for each occurrence, a base-pairing interaction or a mismatch.

In certain embodiments, the structure of formula (IV) does not contain mismatches. In one embodiment, the structure of formula (IV) contains 1 mismatch. In another embodiment, the compound of formula (IV) contains 2 mismatches. In another embodiment, the compound of formula (IV) contains 3 mismatches. In another embodiment, the compound of formula (IV) contains 4 mismatches. In an embodiment, each nucleic acid consists of chemically-modified nucleotides.

In certain embodiments, >95%, >90%, >85%, >80%, >75%, >70%, >65%, >60%, >55% or >50% of X's of the structure of formula (IV) are chemically-modified nucleotides. In other embodiments, >95%, >90%, >85%, >80%, >75%, >70%, >65%, >60%, >55% or >50% of X's of the structure of formula (IV) are chemically-modified nucleotides.

### Structure of Formula (V)

In an embodiment, the compound of formula (I) has the structure of formula (V):

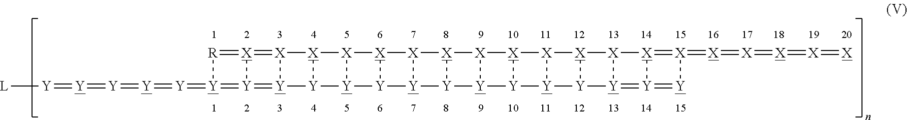

wherein X, for each occurrence, independently, is a nucleotide comprising a 2′-deoxy-2′-fluoro modification; X, for each occurrence, independently, is a nucleotide comprising a 2′-O-methyl modification; Y, for each occurrence, independently, is a nucleotide comprising a 2′-deoxy-2′-fluoro modification; and Y, for each occurrence, independently, is a nucleotide comprising a 2′-O-methyl modification.

In certain embodiments, X is chosen from the group consisting of 2′-deoxy-2′-fluoro modified adenosine, guanosine, uridine or cytidine. In an embodiment, X is chosen from the group consisting of 2′-O-methyl modified adenosine, guanosine, uridine or cytidine. In an embodiment, Y is chosen from the group consisting of 2′-deoxy-2′-fluoro modified adenosine, guanosine, uridine or cytidine. In an embodiment, Y is chosen from the group consisting of 2′-O-methyl modified adenosine, guanosine, uridine or cytidine.

In certain embodiments, the structure of formula (V) does not contain mismatches. In one embodiment, the structure of formula (V) contains 1 mismatch. In another embodiment, the compound of formula (V) contains 2 mismatches. In another embodiment, the compound of formula (V) contains 3 mismatches. In another embodiment, the compound of formula (V) contains 4 mismatches.

### Variable Linkers

In an embodiment of the compound of formula (I), L has the structure of L1:

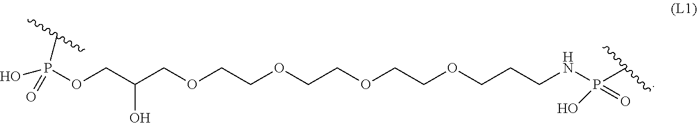 [SMILES: *P(=O)(O)NCCCOCCOCCOCCOCC(O)COP(*)(=O)O]

In an embodiment of L1, R is R³ and n is 2.

In an embodiment of the structure of formula (II), L has the structure of L1. In an embodiment of the structure of formula (III), L has the structure of L1. In an embodiment of the structure of formula (IV), L has the structure of L1. In an embodiment of the structure of formula (V), L has the structure of L1. In an embodiment of the structure of formula (VI), L has the structure of L1. In an embodiment of the structure of formula (VI), L has the structure of L1.

In an embodiment of the compound of formula (I), L has the structure of L2:

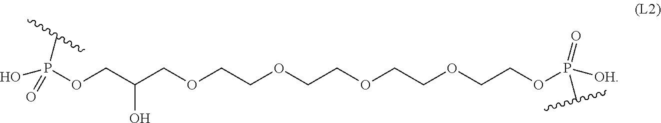 [SMILES: *P(=O)(O)OCCOCCOCCOCCOCC(O)COP(*)(=O)O]

In an embodiment of L2, R is R3 and n is 2. In an embodiment of the structure of formula (II), L has the structure of L2. In an embodiment of the structure of formula (III), L has the structure of L2. In an embodiment of the structure of formula (IV), L has the structure of L2. In an embodiment of the structure of formula (V), L has the structure of L2. In an embodiment of the structure of formula (VI), L has the structure of L2. In an embodiment of the structure of formula (VI), L has the structure of L2.

### Delivery System

In a third aspect, provided herein is a delivery system for therapeutic nucleic acids having the structure of formula (VI):

L-(cNA)ₙ (VI)
wherein L is selected from an ethylene glycol chain, an alkyl chain, a peptide, RNA, DNA, a phosphate, a phosphonate, a phosphoramidate, an ester, an amide, a triazole, and combinations thereof, wherein formula (VI) optionally further comprises one or more branch point B, and one or more spacer S; wherein B is independently for each occurrence a polyvalent organic species or derivative thereof; S is independently for each occurrence selected from an ethylene glycol chain, an alkyl chain, a peptide, RNA, DNA, a phosphate, a phosphonate, a phosphoramidate, an ester, an amide, a triazole, and combinations thereof; each cNA, independently, is a carrier nucleic acid comprising one or more chemical modifications; and n is 2, 3, 4, 5, 6, 7 or 8.

In one embodiment of the delivery system, L is an ethylene glycol chain. In another embodiment of the delivery system, L is an alkyl chain. In another embodiment of the delivery system, L is a peptide. In another embodiment of the delivery system, L is RNA. In another embodiment of the delivery system, L is DNA. In another embodiment of the delivery system, L is a phosphate. In another embodiment of the delivery system, L is a phosphonate. In another embodiment of the delivery system, L is a phosphoramidate. In another embodiment of the delivery system, L is an ester. In another embodiment of the delivery system, L is an amide. In another embodiment of the delivery system, L is a triazole.

In one embodiment of the delivery system, S is an ethylene glycol chain. In another embodiment, S is an alkyl chain. In another embodiment of the delivery system, S is a peptide. In another embodiment, S is RNA. In another embodiment of the delivery system, S is DNA. In another embodiment of the delivery system, S is a phosphate. In another embodiment of the delivery system, S is a phosphonate. In another embodiment of the delivery system, S is a phosphoramidate. In another embodiment of the delivery system, S is an ester. In another embodiment, S is an amide. In another embodiment, S is a triazole.

In one embodiment of the delivery system, n is 2. In another embodiment of the delivery system, n is 3. In another embodiment of the delivery system, n is 4. In another embodiment of the delivery system, n is 5. In another embodiment of the delivery system, n is 6. In another embodiment of the delivery system, n is 7. In another embodiment of the delivery system, n is 8.

In certain embodiments, each cNA comprises >95%, >90%, >85%, >80%, >75%, >70%, >65%, >60%, >55% or >50% chemically-modified nucleotides.

In an embodiment, the compound of formula (VI) has a structure selected from formulas (VI-1)-(VI-9) of Table 3:

TABLE 3

| | |
|---|---|
| 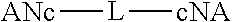 | (VI-1) |
| 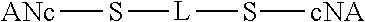 | (VI-2) |
| 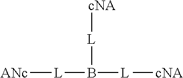 | (VI-3) |
| 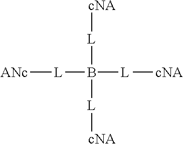 | (VI-4) |
| 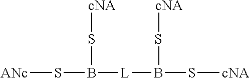 | (VI-5) |
| 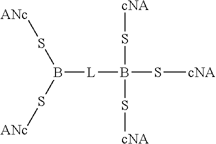 | (VI-6) |
| 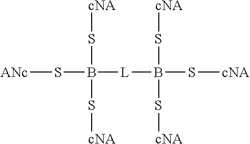 | (VI-7) |
| 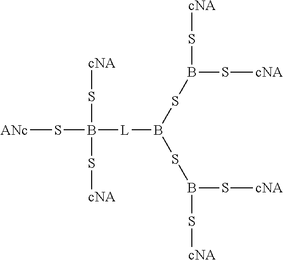 | (VI-8) |
| 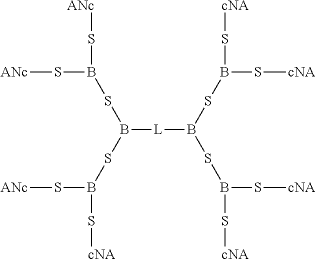 | (VI-9) |

In an embodiment, the compound of formula (VI) is the structure of formula (VI-1). In an embodiment, the compound of formula (VI) is the structure of formula (VI-2). In an embodiment, the compound of formula (VI) is the structure of formula (VI-3). In an embodiment, the compound of formula (VI) is the structure of formula (VI-4). In an embodiment, the compound of formula (VI) is the structure of formula (VI-5). In an embodiment, the compound of formula (VI) is the structure of formula (VI-6). In an embodiment, the compound of formula (VI) is the structure of formula (VI-7). In an embodiment, the compound of formula (VI) is the structure of formula (VI-8). In an embodiment, the compound of formula (VI) is the structure of formula (VI-9).

In an embodiment, the compound of formulas (VI) (including, e.g., formulas (VI-1)-(VI-9), each cNA independently comprises at least 15 contiguous nucleotides. In an embodiment, each cNA independently consists of chemically-modified nucleotides.

In an embodiment, the delivery system further comprises n therapeutic nucleic acids (NA), wherein each NA comprises a sequence substantially complementary to a SARS-CoV-2 nucleic acid sequence of any one of SEQ ID NOs: 1-10, as recited in Table 4 and Table. In further embodiments, NA includes strands that are capable of targeting one or more SARS-CoV-2 nucleic acid sequences of any one of the 45-nucleotide target gene regions recited in Table 6A.

Also, each NA is hybridized to at least one cNA. In one embodiment, the delivery system is comprised of 2 NAs. In another embodiment, the delivery system is comprised of 3 NAs. In another embodiment, the delivery system is comprised of 4 NAs. In another embodiment, the delivery system is comprised of 5 NAs. In another embodiment, the delivery system is comprised of 6 NAs. In another embodiment, the delivery system is comprised of 7 NAs. In another embodiment, the delivery system is comprised of 8 NAs.

In an embodiment, each NA independently comprises at least 15 contiguous nucleotides. In an embodiment, each NA independently comprises 15-25 contiguous nucleotides. In an embodiment, each NA independently comprises 15 contiguous nucleotides. In an embodiment, each NA independently comprises 16 contiguous nucleotides. In another embodiment, each NA independently comprises 17 contiguous nucleotides. In another embodiment, each NA independently comprises 18 contiguous nucleotides. In another embodiment, each NA independently comprises 19 contiguous nucleotides. In another embodiment, each NA independently comprises 20 contiguous nucleotides. In an embodiment, each NA independently comprises 21 contiguous nucleotides. In an embodiment, each NA independently comprises 22 contiguous nucleotides. In an embodiment, each NA independently comprises 23 contiguous nucleotides. In an embodiment, each NA independently comprises 24 contiguous nucleotides. In an embodiment, each NA independently comprises 25 contiguous nucleotides.

In an embodiment, each NA comprises an unpaired overhang of at least 2 nucleotides. In another embodiment, each NA comprises an unpaired overhang of at least 3 nucleotides. In another embodiment, each NA comprises an unpaired overhang of at least 4 nucleotides. In another embodiment, each NA comprises an unpaired overhang of at least 5 nucleotides. In another embodiment, each NA comprises an unpaired overhang of at least 6 nucleotides. In an embodiment, the nucleotides of the overhang are connected via phosphorothioate linkages.

In an embodiment, each NA, independently, is selected from the group consisting of: DNA, siRNAs, antagomiRs, miRNAs, gapmers, mixmers, or guide RNAs. In one embodiment, each NA, independently, is a DNA. In another embodiment, each NA, independently, is a siRNA. In another embodiment, each NA, independently, is an antagomiR. In another embodiment, each NA, independently, is a miRNA. In another embodiment, each NA, independently, is a gapmer. In another embodiment, each NA, independently, is a mixmer. In another embodiment, each NA, independently, is a guide RNA. In an embodiment, each NA is the same. In an embodiment, each NA is not the same.

In an embodiment, the delivery system further comprising n therapeutic nucleic acids (NA) has a structure selected from formulas (I), (II), (III), (IV), (V), (VI), and embodiments thereof described herein. In one embodiment, the delivery system has a structure selected from formulas (I), (II), (III), (IV), (V), (VI), and embodiments thereof described herein further comprising 2 therapeutic nucleic acids (NA). In another embodiment, the delivery system has a structure selected from formulas (I), (II), (III), (IV), (V), (VI), and embodiments thereof described herein further comprising 3 therapeutic nucleic acids (NA). In one embodiment, the delivery system has a structure selected from formulas (I), (II), (III), (IV), (V), (VI), and embodiments thereof described herein further comprising 4 therapeutic nucleic acids (NA). In one embodiment, the delivery system has a structure selected from formulas (I), (II), (III), (IV), (V), (VI), and embodiments thereof described herein further comprising 5 therapeutic nucleic acids (NA). In one embodiment, the delivery system has a structure selected from formulas (I), (II), (III), (IV), (V), (VI), and embodiments thereof described herein further comprising 6 therapeutic nucleic acids (NA). In one embodiment, the delivery system has a structure selected from formulas (I), (II), (III), (IV), (V), (VI), and embodiments thereof described herein further comprising 7 therapeutic nucleic acids (NA). In one embodiment, the delivery system has a structure selected from formulas (I), (II), (III), (IV), (V), (VI), and embodiments thereof described herein further comprising 8 therapeutic nucleic acids (NA).

In one embodiment, the delivery system has a structure selected from formulas (I), (II), (III), (IV), (V), (VI), further comprising a linker of structure L1 or L2 wherein R is R³ and n is 2. In another embodiment, the delivery system has a structure selected from formulas (I), (II), (III), (IV), (V), (VI), further comprising a linker of structure L1 wherein R is R³ and n is 2. In another embodiment, the delivery system has a structure selected from formulas (I), (II), (III), (IV), (V), (VI), further comprising a linker of structure L2 wherein R is R³ and n is 2.

In an embodiment of the delivery system, the target of delivery is selected from the group consisting of: lung, brain, liver, skin, kidney, spleen, pancreas, colon, fat, muscle, adrenal glands, and thymus. In one embodiment, the target of delivery is the lung. In another embodiment, the target of delivery are alveolar cells in the lung. In another embodiment, the target of delivery are club cells in the lung. In another embodiment, the target of delivery is the striatum of the brain. In one embodiment, the target of delivery is the liver. In one embodiment, the target of delivery is the skin. In one embodiment, the target of delivery is the kidney. In one embodiment, the target of delivery is the spleen. In one embodiment, the target of delivery is the pancreas. In one embodiment, the target of delivery is the colon. In one embodiment, the target of delivery is the fat. In one embodiment, the target of delivery are the adrenal glands. In one embodiment, the target of delivery is the muscle. In one embodiment, the target of delivery is the thymus. In one embodiment, the target of delivery is the spinal cord.

In one embodiment, efficacy of delivery to lung cells is achieved through combinations of unique conjugates, optimization of siRNA stability, structural configuration, cleavable linker, and Phosphorothioate (PS) content. In another embodiment, three conjugates, EPA, DCA and PC-DCA, have different distribution profiles. In one embodiment, DCA and PC-DCA are being cleared mostly by the liver and EPA is being cleared mostly by the kidneys. In another embodiment the two classes of conjugates show different cell-type preferences in the lung, where EPA accumulation is higher in epithelial (Clara) cells of the lung.

In certain embodiments, compounds of the invention are characterized by the following properties: (1) two or more branched oligonucleotides, e.g., wherein there is a non-equal number of 3′ and 5′ ends; (2) substantially chemically stabilized, e.g., wherein more than 40%, optimally 100%, of oligonucleotides are chemically modified (e.g., no RNA and optionally no DNA); and (3) phoshorothioated single oligonucleotides containing at least 3, phosphorothioated bonds. In certain embodiments, the phoshorothioated single oligonucleotides contain 4-20 phosphorothioated bonds.

It is to be understood that the methods described in this disclosure are not limited to particular methods and experimental conditions disclosed herein; as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

Branched oligonucleotides, including synthesis and methods of use, are described in greater detail in WO2017/132669, incorporated herein by reference.

### Methods of Introducing Nucleic Acids, Vectors and Host Cells

RNA silencing agents of the invention may be directly introduced into the cell (e.g., a cell in the lung) (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The RNA silencing agents of the invention can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or other-wise increase inhibition of the target gene.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus, the RNA may be introduced along with components that perform one or more of the following activities: enhance RNA uptake by the cell, inhibit annealing of single strands, stabilize the single strands, or other-wise increase inhibition of the target gene.

RNA may be directly introduced into the cell (i.e., intracellularly); or introduced extracellularly into a cavity, interstitial space, into the circulation of an organism, introduced orally, or may be introduced by bathing a cell or organism in a solution containing the RNA. Vascular or extravascular circulation, the blood or lymph system, and the cerebrospinal fluid are sites where the RNA may be introduced.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of double stranded RNA material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, Enzyme Linked ImmunoSorbent Assay (ELISA), Western blotting, RadioImmunoAssay (RIA), other immunoassays, and Fluorescence Activated Cell Sorting (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracycline. Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of RNAi agent may result in inhibition in a smaller fraction of cells (e.g., at least 10%, 20%, 50%, 75%, 90%, or 95% of targeted cells). Quantization of gene expression in a cell may show similar amounts of inhibition at the level of accumulation of target mRNA or translation of target protein. As an example, the efficiency of inhibition may be determined by assessing the amount of gene product in the cell; mRNA may be detected with a hybridization probe having a nucleotide sequence outside the region used for the inhibitory double-stranded RNA, or translated polypeptide may be detected with an antibody raised against the polypeptide sequence of that region.

The RNA may be introduced in an amount which allows delivery of at least one copy per cell. Higher doses (e.g., at least 5, 10, 100, 500 or 1000 copies per cell) of material may yield more effective inhibition; lower doses may also be useful for specific applications.

In an exemplary aspect, the efficacy of an RNAi agent of the invention (e.g., an siRNA targeting an SARS-CoV-2 target sequence) is tested for its ability to specifically degrade mutant mRNA (e.g., SARS-CoV-2 mRNA and/or the production of SARS-CoV-2 protein) in cells, such as cells in the lung. In certain embodiments, cells in the lung include, but are not limited to, clara cells, alveolar cells, and club cells. Also suitable for cell-based validation assays are other readily transfectable cells, for example, HeLa cells or COS cells. Cells are transfected with human wild type or mutant cDNAs (e.g., human wild type or mutant SARS-CoV-2 cDNA). Standard siRNA, modified siRNA or vectors able to produce siRNA from U-looped mRNA are co-transfected. Selective reduction in target mRNA (e.g., SARS-CoV-2 mRNA) and/or target protein (e.g., SARS-CoV-2 protein) is measured. Reduction of target mRNA or protein can be compared to levels of target mRNA or protein in the absence of an RNAi agent or in the presence of an RNAi agent that does not target SARS-CoV-2 mRNA. Exogenously-introduced mRNA or protein (or endogenous mRNA or protein) can be assayed for comparison purposes. When utilizing lung cells, it may be desirable to introduce RNAi agents (e.g., siRNAs) by passive uptake.

### Recombinant Adeno-Associated Viruses and Vectors

In certain exemplary embodiments, recombinant adeno-associated viruses (rAAVs) and their associated vectors can be used to deliver one or more siRNAs into cells, e.g., lung cells (e.g., clara cells, alveolar cells or club cells). AAV is able to infect many different cell types, although the infection efficiency varies based upon serotype, which is determined by the sequence of the capsid protein. Several native AAV serotypes have been identified, with serotypes 1-9 being the most commonly used for recombinant AAV. AAV-2 is the most well-studied and published serotype. The AAV-DJ system includes serotypes AAV-DJ and AAV-DJ/8. These serotypes were created through DNA shuffling of multiple AAV serotypes to produce AAV with hybrid capsids that have improved transduction efficiencies in vitro (AAV-DJ) and in vivo (AAV-DJ/8) in a variety of cells and tissues.

In certain embodiments, widespread lung delivery can be achieved by intratracheal (IT) delivery of recombinant adeno-associated virus 7 (rAAV7), RAAV9 and rAAV10, or other suitable rAAVs. rAAVs and their associated vectors are well-known in the art and are described in US Patent Applications 2014/0296486, 2010/0186103, 2008/0269149, 2006/0078542 and 2005/0220766, each of which is incorporated herein by reference in its entirety for all purposes.

rAAVs may be delivered to a subject in compositions according to any appropriate methods known in the art. An rAAV can be suspended in a physiologically compatible carrier (i.e., in a composition), and may be administered to a subject, i.e., a host animal, such as a human, mouse, rat, cat, dog, sheep, rabbit, horse, cow, goat, pig, guinea pig, hamster, chicken, turkey, a non-human primate (e.g., Macaque) or the like. In certain embodiments, a host animal is a non-human host animal.

Delivery of one or more rAAVs to a mammalian subject may be performed, for example, by intramuscular injection or by administration into the bloodstream of the mammalian subject. Administration into the bloodstream may be by injection into a vein, an artery, or any other vascular conduit. In certain embodiments, one or more rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer virions into the vasculature of an isolated limb to potentially enhance transduction into muscle cells or tissue. Moreover, in certain instances, it may be desirable to deliver virions to the lung of a subject. By “lung” is meant all cells and tissue of the lung of a vertebrate. Thus, the term includes, but is not limited to, clara cells, alveolar cells, club cells, and the like. Recombinant AAVs may be delivered directly to the lung by injection, e.g., intratracheal injection.

The compositions of the invention may comprise an rAAV alone, or in combination with one or more other viruses (e.g., a second rAAV encoding having one or more different transgenes). In certain embodiments, a composition comprises 1, 2, 3, 4, 5, 6, 7, 8, 9, 10 or more different rAAVs each having one or more different transgenes.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of one or more rAAVs is generally in the range of from about 1 ml to about 100 ml of solution containing from about 10⁹ to 10¹⁶ genome copies. In some cases, a dosage between about 10¹¹ to 10¹² rAAV genome copies is appropriate. In certain embodiments, 10¹² rAAV genome copies is effective to target lung, heart, liver, and pancreas tissues. In some cases, stable transgenic animals are produced by multiple doses of an rAAV.

In some embodiments, rAAV compositions are formulated to reduce aggregation of AAV particles in the composition, particularly where high rAAV concentrations are present (e.g., about 10¹³ genome copies/mL or more). Methods for reducing aggregation of rAAVs are well known in the art and, include, for example, addition of surfactants, pH adjustment, salt concentration adjustment, etc. (See, e.g., Wright et al. (2005) Molecular Therapy 12:171-178, the contents of which are incorporated herein by reference.)

“Recombinant AAV (rAAV) vectors” comprise, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., siRNA) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are usually about 145 basepairs in length. In certain embodiments, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including mammalian AAV types described further herein.

### VIII. Methods of Treatment

In one aspect, the present invention provides for both prophylactic and therapeutic methods of treating a subject at risk of (or susceptible to) SARS-CoV-2 infection. In one embodiment, the disease or disorder is such that SARS-CoV-2 levels in blood or another biological sample have been found to be a marker of infection. In another embodiment, the infection with SARS-CoV-2 is characterized by a clinical manifestation of viral infection, e.g. an increase in body temperature. In a certain embodiment, a reduction in SARS-CoV-2 mRNA reduces clinical manifestations of SARS-CoV-2 infection.

“Treatment,” or “treating,” as used herein, is defined as the application or administration of a therapeutic agent (e.g., a RNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., an RNAi agent or vector or transgene encoding same). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the invention pertains to methods treating subjects therapeutically, i.e., alter onset of symptoms of the disease or disorder. In an exemplary embodiment, the modulatory method of the invention involves contacting a lung cell with a therapeutic agent (e.g., a RNAi agent or vector or transgene encoding same) that is specific for a target sequence within the gene (e.g., SARS-CoV-2 target sequences of Tables 4, 5, 6A, 7, and 8), such that sequence specific interference with the gene is achieved. These methods can be performed in vitro (e.g., by culturing the cell with the agent) or, alternatively, in vivo (e.g., by administering the agent to a subject).

### IX. Pharmaceutical Compositions and Methods of Administration

The invention pertains to uses of the above-described agents for prophylactic and/or therapeutic treatments as described infra. Accordingly, the modulators (e.g., RNAi agents) of the present invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration.

The nucleic acid molecules of the invention can be inserted into expression constructs, e.g., viral vectors, retroviral vectors, expression cassettes, or plasmid viral vectors, e.g., using methods known in the art, including but not limited to those described in Xia et al., (2002), Supra. Expression constructs can be delivered to a subject by, for example, inhalation, orally, intravenous injection, local administration (see U.S. Pat. No. 5,328,470) or by stereotactic injection (see e.g., Chen et al. (1994), Proc. Natl. Acad. Sci. USA, 91, 3054-3057). The pharmaceutical preparation of the delivery vector can include the vector in an acceptable diluent, or can comprise a slow release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The nucleic acid molecules of the invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides. Brummelkamp et al. (2002), Science, 296, 550-553; Lee et al, (2002). supra; Miyagishi and Taira (2002), Nature Biotechnol., 20, 497-500; Paddison et al. (2002), supra; Paul (2002), supra; Sui (2002) supra; Yu et al. (2002), supra.

The expression constructs may be any construct suitable for use in the appropriate expression system and include, but are not limited to retroviral vectors, linear expression cassettes, plasmids and viral or virally-derived vectors, as known in the art. Such expression constructs may include one or more inducible promoters, RNA Pol III promoter systems such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct, Tuschl (2002), Supra.

In certain embodiments, a composition that includes a compound of the invention can be delivered to the lungs of a subject by a variety of routes. Exemplary routes include intratracheal or nasal delivery. The composition can also be delivered systemically, e.g., by intravenous, subcutaneous or intramuscular injection.

For example, compositions can include one or more species of a compound of the invention and a pharmaceutically acceptable carrier. The pharmaceutical compositions of the present invention may be administered in a number of ways depending upon whether local or systemic treatment is desired and upon the area to be treated.

As used herein “intratracheal administration” refers to the direct administration to the lung through the trachea. Intratracheal administration includes, but is not limited to, intratracheal inhalation and intratracheal instillation. Intratracheal administration (IT) is non-invasive and can be used in the field. Both the chemical architectures of the siRNAs and routes of administration might have benefits in different clinical contexts and disease stages. The chemical architectures optimal for different routes of administration is different. For example, DCA-conjugated siRNAs or divalent siRNAs can be effective for delivery of siRNA to the lung.

In one embodiment, divalent siRNAs are delivered to lung tissues. A variety of lung delivery systems can be employed to accomplish delivery to the lung tissues, for example, but not limited to, direct intrathecal instillation, or by using a nebulizer. Formulations in which the siRNA can be delivered are, for example, but not limited to, dry powder, direct powder, vapor droplets, etc.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following example, which is included for purposes of illustration only and is not intended to be limiting.

### EXAMPLES

### Example 1. In Vitro Identification of SARS-CoV-2 Targeting Sequences

Hyper functional siRNAs targeting all 9 genes (regions) of SARS-CoV2 were identified. A combination of bioinformatic approaches were employed to identify regions of conservation (based on 718 patients isolates) in combination with features essential for RISC entry and tolerance of chemical modifications. Over 100 chemically optimized compounds were synthesized, and reporter systems developed to test these compounds in cells, screened at 1.5 μM concentration. Selected hits were further screened in dose response studies, and at least two lead compounds were identified per gene with IC₅₀ values <20 nM.

Target sequences were derived from the severe acute respiratory syndrome coronavirus 2 (SARS CoV-2) isolate Wuhan-Hu-1 (NCBI accession: NC_045512). Fully chemically modified and conjugated oligonucleotides targeting SARS-CoV-2 and the human host receptors of SARS-CoV2 could potentially prevent and treat viral infections from viruses within the family Coronaviridiae.

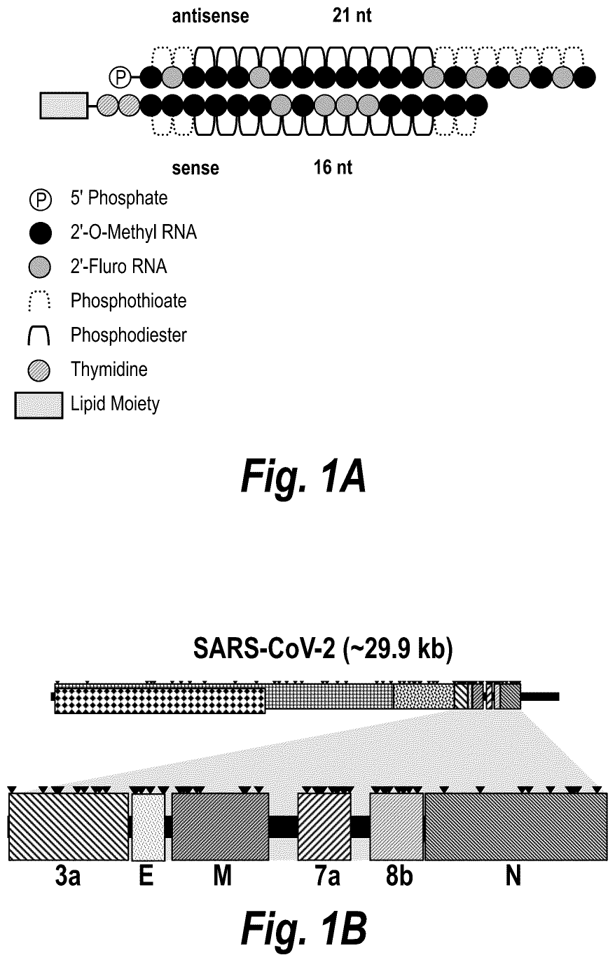

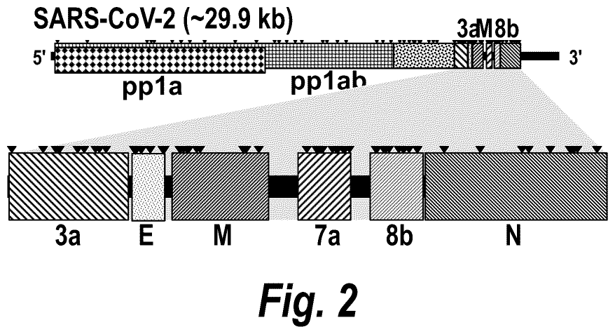

Nine SARS-CoV-2 transcripts were selected for knockdown: the genes coding for the four major structural proteins spike surface glycoprotein (S), small envelope protein (E), matrix protein (M), and nucleocapsid protein (N), as well as genes coding for pp1a, pp1ab, which make up the 16 non-structural proteins of SARS-CoV-2, and genes coding for the accessory proteins 3a, 8b, 7a (FIG. 1). Table 4 shows the full-length sequence of the SARS-CoV-2 genome, Table 5 the sequences of the SARS-CoV-2 genes. FIG. 2 depicts a diagram of siRNA and antisense oligonucleotides (ASOs) target positions on encoded proteins in the SARS-CoV-2 genome. siRNAs were designed to target nine genes encoding SARS-CoV-2 proteins: orf1a, orf1ab, spike surface glycoprotein (S), small envelope protein (E), matrix protein (M), nucleocapsid protein (N), and accessory proteins 3a, 8b, 7a. Grey arrows indicate siRNA and ASO target positions. Inset in FIG. 2 shows a detailed view of siRNA target positions on genes in the 3′ region of the genome.

TABLE 4
SARS-CoV2 genomic sequence (NCBI Accession Number
NC_045512)

SARS-CoV2 genome sequence
ATTAAAGGTTTATACCTTCCCAGGTAACAAACCAACCAACTTTCGATCTC
TTGTAGATCTGTTCTCTAAACGAACTTTAAAATCTGTGTGGCTGTCACTC
GGCTGCATGCTTAGTGCACTCACGCAGTATAATTAATAACTAATTACTGT
CGTTGACAGGACACGAGTAACTCGTCTATCTTCTGCAGGCTGCTTACGGT
TTCGTCCGTGTTGCAGCCGATCATCAGCACATCTAGGTTTCGTCCGGGTG
TGACCGAAAGGTAAGATGGAGAGCCTTGTCCCTGGTTTCAACGAGAAAAC
ACACGTCCAACTCAGTTTGCCTGTTTTACAGGTTCGCGACGTGCTCGTAC
GTGGCTTTGGAGACTCCGTGGAGGAGGTCTTATCAGAGGCACGTCAACAT
CTTAAAGATGGCACTTGTGGCTTAGTAGAAGTTGAAAAAGGCGTTTTGCC
TCAACTTGAACAGCCCTATGTGTTCATCAAACGTTCGGATGCTCGAACTG
CACCTCATGGTCATGTTATGGTTGAGCTGGTAGCAGAACTCGAAGGCATT
CAGTACGGTCGTAGTGGTGAGACACTTGGTGTCCTTGTCCCTCATGTGGG
CGAAATACCAGTGGCTTACCGCAAGGTTCTTCTTCGTAAGAACGGTAATA
AAGGAGCTGGTGGCCATAGTTACGGCGCCGATCTAAAGTCATTTGACTTA
GGCGACGAGCTTGGCACTGATCCTTATGAAGATTTTCAAGAAAACTGGAA
CACTAAACATAGCAGTGGTGTTACCCGTGAACTCATGCGTGAGCTTAACG
GAGGGGCATACACTCGCTATGTCGATAACAACTTCTGTGGCCCTGATGGC
TACCCTCTTGAGTGCATTAAAGACCTTCTAGCACGTGCTGGTAAAGCTTC
ATGCACTTTGTCCGAACAACTGGACTTTATTGACACTAAGAGGGGTGTAT
ACTGCTGCCGTGAACATGAGCATGAAATTGCTTGGTACACGGAACGTTCT
GAAAAGAGCTATGAATTGCAGACACCTTTTGAAATTAAATTGGCAAAGAA
ATTTGACACCTTCAATGGGGAATGTCCAAATTTTGTATTTCCCTTAAATT
CCATAATCAAGACTATTCAACCAAGGGTTGAAAAGAAAAAGCTTGATGGC
TTTATGGGTAGAATTCGATCTGTCTATCCAGTTGCGTCACCAAATGAATG
CAACCAAATGTGCCTTTCAACTCTCATGAAGTGTGATCATTGTGGTGAAA
CTTCATGGCAGACGGGCGATTTTGTTAAAGCCACTTGCGAATTTTGTGGC
ACTGAGAATTTGACTAAAGAAGGTGCCACTACTTGTGGTTACTTACCCCA
AAATGCTGTTGTTAAAATTTATTGTCCAGCATGTCACAATTCAGAAGTAG
GACCTGAGCATAGTCTTGCCGAATACCATAATGAATCTGGCTTGAAAACC
ATTCTTCGTAAGGGTGGTCGCACTATTGCCTTTGGAGGCTGTGTGTTCTC
TTATGTTGGTTGCCATAACAAGTGTGCCTATTGGGTTCCACGTGCTAGCG
CTAACATAGGTTGTAACCATACAGGTGTTGTTGGAGAAGGTTCCGAAGGT
CTTAATGACAACCTTCTTGAAATACTCCAAAAAGAGAAAGTCAACATCAA
TATTGTTGGTGACTTTAAACTTAATGAAGAGATCGCCATTATTTTGGCAT
CTTTTTCTGCTTCCACAAGTGCTTTTGTGGAAACTGTGAAAGGTTTGGAT
TATAAAGCATTCAAACAAATTGTTGAATCCTGTGGTAATTTTAAAGTTAC
AAAAGGAAAAGCTAAAAAAGGTGCCTGGAATATTGGTGAACAGAAATCAA
TACTGAGTCCTCTTTATGCATTTGCATCAGAGGCTGCTCGTGTTGTACGA
TCAATTTTCTCCCGCACTCTTGAAACTGCTCAAAATTCTGTGCGTGTTTT
ACAGAAGGCCGCTATAACAATACTAGATGGAATTTCACAGTATTCACTGA
GACTCATTGATGCTATGATGTTCACATCTGATTTGGCTACTAACAATCTA
GTTGTAATGGCCTACATTACAGGTGGTGTTGTTCAGTTGACTTCGCAGTG
GCTAACTAACATCTTTGGCACTGTTTATGAAAAACTCAAACCCGTCCTTG
ATTGGCTTGAAGAGAAGTTTAAGGAAGGTGTAGAGTTTCTTAGAGACGGT
TGGGAAATTGTTAAATTTATCTCAACCTGTGCTTGTGAAATTGTCGGTGG
ACAAATTGTCACCTGTGCAAAGGAAATTAAGGAGAGTGTTCAGACATTCT
TTAAGCTTGTAAATAAATTTTTGGCTTTGTGTGCTGACTCTATCATTATT
GGTGGAGCTAAACTTAAAGCCTTGAATTTAGGTGAAACATTTGTCACGCA
CTCAAAGGGATTGTACAGAAAGTGTGTTAAATCCAGAGAAGAAACTGGCC
TACTCATGCCTCTAAAAGCCCCAAAAGAAATTATCTTCTTAGAGGGAGAA
ACACTTCCCACAGAAGTGTTAACAGAGGAAGTTGTCTTGAAAACTGGTGA
TTTACAACCATTAGAACAACCTACTAGTGAAGCTGTTGAAGCTCCATTGG
TTGGTACACCAGTTTGTATTAACGGGCTTATGTTGCTCGAAATCAAAGAC
ACAGAAAAGTACTGTGCCCTTGCACCTAATATGATGGTAACAAACAATAC
CTTCACACTCAAAGGCGGTGCACCAACAAAGGTTACTTTTGGTGATGACA
CTGTGATAGAAGTGCAAGGTTACAAGAGTGTGAATATCACTTTTGAACTT
GATGAAAGGATTGATAAAGTACTTAATGAGAAGTGCTCTGCCTATACAGT
TGAACTCGGTACAGAAGTAAATGAGTTCGCCTGTGTTGTGGCAGATGCTG
TCATAAAAACTTTGCAACCAGTATCTGAATTACTTACACCACTGGGCATT
GATTTAGATGAGTGGAGTATGGCTACATACTACTTATTTGATGAGTCTGG
TGAGTTTAAATTGGCTTCACATATGTATTGTTCTTTCTACCCTCCAGATG
AGGATGAAGAAGAAGGTGATTGTGAAGAAGAAGAGTTTGAGCCATCAACT
CAATATGAGTATGGTACTGAAGATGATTACCAAGGTAAACCTTTGGAATT
TGGTGCCACTTCTGCTGCTCTTCAACCTGAAGAAGAGCAAGAAGAAGATT
GGTTAGATGATGATAGTCAACAAACTGTTGGTCAACAAGACGGCAGTGAG
GACAATCAGACAACTACTATTCAAACAATTGTTGAGGTTCAACCTCAATT
AGAGATGGAACTTACACCAGTTGTTCAGACTATTGAAGTGAATAGTTTTA
GTGGTTATTTAAAACTTACTGACAATGTATACATTAAAAATGCAGACATT
GTGGAAGAAGCTAAAAAGGTAAAACCAACAGTGGTTGTTAATGCAGCCAA
TGTTTACCTTAAACATGGAGGAGGTGTTGCAGGAGCCTTAAATAAGGCTA
CTAACAATGCCATGCAAGTTGAATCTGATGATTACATAGCTACTAATGGA
CCACTTAAAGTGGGTGGTAGTTGTGTTTTAAGCGGACACAATCTTGCTAA
ACACTGTCTTCATGTTGTCGGCCCAAATGTTAACAAAGGTGAAGACATTC
AACTTCTTAAGAGTGCTTATGAAAATTTTAATCAGCACGAAGTTCTACTT
GCACCATTATTATCAGCTGGTATTTTTGGTGCTGACCCTATACATTCTTT
AAGAGTTTGTGTAGATACTGTTCGCACAAATGTCTACTTAGCTGTCTTTG
ATAAAAATCTCTATGACAAACTTGTTTCAAGCTTTTTGGAAATGAAGAGT
GAAAAGCAAGTTGAACAAAAGATCGCTGAGATTCCTAAAGAGGAAGTTAA
GCCATTTATAACTGAAAGTAAACCTTCAGTTGAACAGAGAAAACAAGATG
ATAAGAAAATCAAAGCTTGTGTTGAAGAAGTTACAACAACTCTGGAAGAA
ACTAAGTTCCTCACAGAAAACTTGTTACTTTATATTGACATTAATGGCAA
TCTTCATCCAGATTCTGCCACTCTTGTTAGTGACATTGACATCACTTTCT
TAAAGAAAGATGCTCCATATATAGTGGGTGATGTTGTTCAAGAGGGTGTT
TTAACTGCTGTGGTTATACCTACTAAAAAGGCTGGTGGCACTACTGAAAT
GCTAGCGAAAGCTTTGAGAAAAGTGCCAACAGACAATTATATAACCACTT
ACCCGGGTCAGGGTTTAAATGGTTACACTGTAGAGGAGGCAAAGACAGTG
CTTAAAAAGTGTAAAAGTGCCTTTTACATTCTACCATCTATTATCTCTAA
TGAGAAGCAAGAAATTCTTGGAACTGTTTCTTGGAATTTGCGAGAAATGC
TTGCACATGCAGAAGAAACACGCAAATTAATGCCTGTCTGTGTGGAAACT
AAAGCCATAGTTTCAACTATACAGCGTAAATATAAGGGTATTAAAATACA
AGAGGGTGTGGTTGATTATGGTGCTAGATTTTACTTTTACACCAGTAAAA
CAACTGTAGCGTCACTTATCAACACACTTAACGATCTAAATGAAACTCTT
GTTACAATGCCACTTGGCTATGTAACACATGGCTTAAATTTGGAAGAAGC
TGCTCGGTATATGAGATCTCTCAAAGTGCCAGCTACAGTTTCTGTTTCTT
CACCTGATGCTGTTACAGCGTATAATGGTTATCTTACTTCTTCTTCTAAA
ACACCTGAAGAACATTTTATTGAAACCATCTCACTTGCTGGTTCCTATAA
AGATTGGTCCTATTCTGGACAATCTACACAACTAGGTATAGAATTTCTTA
AGAGAGGTGATAAAAGTGTATATTACACTAGTAATCCTACCACATTCCAC
CTAGATGGTGAAGTTATCACCTTTGACAATCTTAAGACACTTCTTTCTTT
GAGAGAAGTGAGGACTATTAAGGTGTTTACAACAGTAGACAACATTAACC
TCCACACGCAAGTTGTGGACATGTCAATGACATATGGACAACAGTTTGGT
CCAACTTATTTGGATGGAGCTGATGTTACTAAAATAAAACCTCATAATTC
ACATGAAGGTAAAACATTTTATGTTTTACCTAATGATGACACTCTACGTG
TTGAGGCTTTTGAGTACTACCACACAACTGATCCTAGTTTTCTGGGTAGG
TACATGTCAGCATTAAATCACACTAAAAAGTGGAAATACCCACAAGTTAA
TGGTTTAACTTCTATTAAATGGGCAGATAACAACTGTTATCTTGCCACTG
CATTGTTAACACTCCAACAAATAGAGTTGAAGTTTAATCCACCTGCTCTA
CAAGATGCTTATTACAGAGCAAGGGCTGGTGAAGCTGCTAACTTTTGTGC
ACTTATCTTAGCCTACTGTAATAAGACAGTAGGTGAGTTAGGTGATGTTA
GAGAAACAATGAGTTACTTGTTTCAACATGCCAATTTAGATTCTTGCAAA
AGAGTCTTGAACGTGGTGTGTAAAACTTGTGGACAACAGCAGACAACCCT
TAAGGGTGTAGAAGCTGTTATGTACATGGGCACACTTTCTTATGAACAAT
TTAAGAAAGGTGTTCAGATACCTTGTACGTGTGGTAAACAAGCTACAAAA
TATCTAGTACAACAGGAGTCACCTTTTGTTATGATGTCAGCACCACCTGC
TCAGTATGAACTTAAGCATGGTACATTTACTTGTGCTAGTGAGTACACTG
GTAATTACCAGTGTGGTCACTATAAACATATAACTTCTAAAGAAACTTTG
TATTGCATAGACGGTGCTTTACTTACAAAGTCCTCAGAATACAAAGGTCC
TATTACGGATGTTTTCTACAAAGAAAACAGTTACACAACAACCATAAAAC
CAGTTACTTATAAATTGGATGGTGTTGTTTGTACAGAAATTGACCCTAAG
TTGGACAATTATTATAAGAAAGACAATTCTTATTTCACAGAGCAACCAAT
TGATCTTGTACCAAACCAACCATATCCAAACGCAAGCTTCGATAATTTTA
AGTTTGTATGTGATAATATCAAATTTGCTGATGATTTAAACCAGTTAACT
GGTTATAAGAAACCTGCTTCAAGAGAGCTTAAAGTTACATTTTTCCCTGA
CTTAAATGGTGATGTGGTGGCTATTGATTATAAACACTACACACCCTCTT
TTAAGAAAGGAGCTAAATTGTTACATAAACCTATTGTTTGGCATGTTAAC
AATGCAACTAATAAAGCCACGTATAAACCAAATACCTGGTGTATACGTTG
TCTTTGGAGCACAAAACCAGTTGAAACATCAAATTCGTTTGATGTACTGA
AGTCAGAGGACGCGCAGGGAATGGATAATCTTGCCTGCGAAGATCTAAAA
CCAGTCTCTGAAGAAGTAGTGGAAAATCCTACCATACAGAAAGACGTTCT
TGAGTGTAATGTGAAAACTACCGAAGTTGTAGGAGACATTATACTTAAAC
CAGCAAATAATAGTTTAAAAATTACAGAAGAGGTTGGCCACACAGATCTA
ATGGCTGCTTATGTAGACAATTCTAGTCTTACTATTAAGAAACCTAATGA
ATTATCTAGAGTATTAGGTTTGAAAACCCTTGCTACTCATGGTTTAGCTG
CTGTTAATAGTGTCCCTTGGGATACTATAGCTAATTATGCTAAGCCTTTT
CTTAACAAAGTTGTTAGTACAACTACTAACATAGTTACACGGTGTTTAAA
CCGTGTTTGTACTAATTATATGCCTTATTTCTTTACTTTATTGCTACAAT
TGTGTACTTTTACTAGAAGTACAAATTCTAGAATTAAAGCATCTATGCCG
ACTACTATAGCAAAGAATACTGTTAAGAGTGTCGGTAAATTTTGTCTAGA
GGCTTCATTTAATTATTTGAAGTCACCTAATTTTTCTAAACTGATAAATA
TTATAATTTGGTTTTTACTATTAAGTGTTTGCCTAGGTTCTTTAATCTAC
TCAACCGCTGCTTTAGGTGTTTTAATGTCTAATTTAGGCATGCCTTCTTA
CTGTACTGGTTACAGAGAAGGCTATTTGAACTCTACTAATGTCACTATTG
CAACCTACTGTACTGGTTCTATACCTTGTAGTGTTTGTCTTAGTGGTTTA
GATTCTTTAGACACCTATCCTTCTTTAGAAACTATACAAATTACCATTTC
ATCTTTTAAATGGGATTTAACTGCTTTTGGCTTAGTTGCAGAGTGGTTTT
TGGCATATATTCTTTTCACTAGGTTTTTCTATGTACTTGGATTGGCTGCA
ATCATGCAATTGTTTTTCAGCTATTTTGCAGTACATTTTATTAGTAATTC
TTGGCTTATGTGGTTAATAATTAATCTTGTACAAATGGCCCCGATTTCAG
CTATGGTTAGAATGTACATCTTCTTTGCATCATTTTATTATGTATGGAAA
AGTTATGTGCATGTTGTAGACGGTTGTAATTCATCAACTTGTATGATGTG
TTACAAACGTAATAGAGCAACAAGAGTCGAATGTACAACTATTGTTAATG
GTGTTAGAAGGTCCTTTTATGTCTATGCTAATGGAGGTAAAGGCTTTTGC
AAACTACACAATTGGAATTGTGTTAATTGTGATACATTCTGTGCTGGTAG
TACATTTATTAGTGATGAAGTTGCGAGAGACTTGTCACTACAGTTTAAAA
GACCAATAAATCCTACTGACCAGTCTTCTTACATCGTTGATAGTGTTACA
GTGAAGAATGGTTCCATCCATCTTTACTTTGATAAAGCTGGTCAAAAGAC
TTATGAAAGACATTCTCTCTCTCATTTTGTTAACTTAGACAACCTGAGAG
CTAATAACACTAAAGGTTCATTGCCTATTAATGTTATAGTTTTTGATGGT
AAATCAAAATGTGAAGAATCATCTGCAAAATCAGCGTCTGTTTACTACAG
TCAGCTTATGTGTCAACCTATACTGTTACTAGATCAGGCATTAGTGTCTG
ATGTTGGTGATAGTGCGGAAGTTGCAGTTAAAATGTTTGATGCTTACGTT
AATACGTTTTCATCAACTTTTAACGTACCAATGGAAAAACTCAAAACACT
AGTTGCAACTGCAGAAGCTGAACTTGCAAAGAATGTGTCCTTAGACAATG
TCTTATCTACTTTTATTTCAGCAGCTCGGCAAGGGTTTGTTGATTCAGAT
GTAGAAACTAAAGATGTTGTTGAATGTCTTAAATTGTCACATCAATCTGA
CATAGAAGTTACTGGCGATAGTTGTAATAACTATATGCTCACCTATAACA
AAGTTGAAAACATGACACCCCGTGACCTTGGTGCTTGTATTGACTGTAGT
GCGCGTCATATTAATGCGCAGGTAGCAAAAAGTCACAACATTGCTTTGAT
ATGGAACGTTAAAGATTTCATGTCATTGTCTGAACAACTACGAAAACAAA
TACGTAGTGCTGCTAAAAAGAATAACTTACCTTTTAAGTTGACATGTGCA
ACTACTAGACAAGTTGTTAATGTTGTAACAACAAAGATAGCACTTAAGGG
TGGTAAAATTGTTAATAATTGGTTGAAGCAGTTAATTAAAGTTACACTTG
TGTTCCTTTTTGTTGCTGCTATTTTCTATTTAATAACACCTGTTCATGTC
ATGTCTAAACATACTGACTTTTCAAGTGAAATCATAGGATACAAGGCTAT
TGATGGTGGTGTCACTCGTGACATAGCATCTACAGATACTTGTTTTGCTA
ACAAACATGCTGATTTTGACACATGGTTTAGCCAGCGTGGTGGTAGTTAT
ACTAATGACAAAGCTTGCCCATTGATTGCTGCAGTCATAACAAGAGAAGT
GGGTTTTGTCGTGCCTGGTTTGCCTGGCACGATATTACGCACAACTAATG
GTGACTTTTTGCATTTCTTACCTAGAGTTTTTAGTGCAGTTGGTAACATC
TGTTACACACCATCAAAACTTATAGAGTACACTGACTTTGCAACATCAGC
TTGTGTTTTGGCTGCTGAATGTACAATTTTTAAAGATGCTTCTGGTAAGC
CAGTACCATATTGTTATGATACCAATGTACTAGAAGGTTCTGTTGCTTAT
GAAAGTTTACGCCCTGACACACGTTATGTGCTCATGGATGGCTCTATTAT
TCAATTTCCTAACACCTACCTTGAAGGTTCTGTTAGAGTGGTAACAACTT
TTGATTCTGAGTACTGTAGGCACGGCACTTGTGAAAGATCAGAAGCTGGT
GTTTGTGTATCTACTAGTGGTAGATGGGTACTTAACAATGATTATTACAG
ATCTTTACCAGGAGTTTTCTGTGGTGTAGATGCTGTAAATTTACTTACTA
ATATGTTTACACCACTAATTCAACCTATTGGTGCTTTGGACATATCAGCA
TCTATAGTAGCTGGTGGTATTGTAGCTATCGTAGTAACATGCCTTGCCTA
CTATTTTATGAGGTTTAGAAGAGCTTTTGGTGAATACAGTCATGTAGTTG
CCTTTAATACTTTACTATTCCTTATGTCATTCACTGTACTCTGTTTAACA
CCAGTTTACTCATTCTTACCTGGTGTTTATTCTGTTATTTACTTGTACTT
GACATTTTATCTTACTAATGATGTTTCTTTTTTAGCACATATTCAGTGGA
TGGTTATGTTCACACCTTTAGTACCTTTCTGGATAACAATTGCTTATATC
ATTTGTATTTCCACAAAGCATTTCTATTGGTTCTTTAGTAATTACCTAAA
GAGACGTGTAGTCTTTAATGGTGTTTCCTTTAGTACTTTTGAAGAAGCTG
CGCTGTGCACCTTTTTGTTAAATAAAGAAATGTATCTAAAGTTGCGTAGT
GATGTGCTATTACCTCTTACGCAATATAATAGATACTTAGCTCTTTATAA
TAAGTACAAGTATTTTAGTGGAGCAATGGATACAACTAGCTACAGAGAAG
CTGCTTGTTGTCATCTCGCAAAGGCTCTCAATGACTTCAGTAACTCAGGT
TCTGATGTTCTTTACCAACCACCACAAACCTCTATCACCTCAGCTGTTTT
GCAGAGTGGTTTTAGAAAAATGGCATTCCCATCTGGTAAAGTTGAGGGTT
GTATGGTACAAGTAACTTGTGGTACAACTACACTTAACGGTCTTTGGCTT
GATGACGTAGTTTACTGTCCAAGACATGTGATCTGCACCTCTGAAGACAT
GCTTAACCCTAATTATGAAGATTTACTCATTCGTAAGTCTAATCATAATT
TCTTGGTACAGGCTGGTAATGTTCAACTCAGGGTTATTGGACATTCTATG
CAAAATTGTGTACTTAAGCTTAAGGTTGATACAGCCAATCCTAAGACACC
TAAGTATAAGTTTGTTCGCATTCAACCAGGACAGACTTTTTCAGTGTTAG
CTTGTTACAATGGTTCACCATCTGGTGTTTACCAATGTGCTATGAGGCCC
AATTTCACTATTAAGGGTTCATTCCTTAATGGTTCATGTGGTAGTGTTGG
TTTTAACATAGATTATGACTGTGTCTCTTTTTGTTACATGCACCATATGG
AATTACCAACTGGAGTTCATGCTGGCACAGACTTAGAAGGTAACTTTTAT
GGACCTTTTGTTGACAGGCAAACAGCACAAGCAGCTGGTACGGACACAAC
TATTACAGTTAATGTTTTAGCTTGGTTGTACGCTGCTGTTATAAATGGAG
ACAGGTGGTTTCTCAATCGATTTACCACAACTCTTAATGACTTTAACCTT
GTGGCTATGAAGTACAATTATGAACCTCTAACACAAGACCATGTTGACAT
ACTAGGACCTCTTTCTGCTCAAACTGGAATTGCCGTTTTAGATATGTGTG
CTTCATTAAAAGAATTACTGCAAAATGGTATGAATGGACGTACCATATTG
GGTAGTGCTTTATTAGAAGATGAATTTACACCTTTTGATGTTGTTAGACA
ATGCTCAGGTGTTACTTTCCAAAGTGCAGTGAAAAGAACAATCAAGGGTA
CACACCACTGGTTGTTACTCACAATTTTGACTTCACTTTTAGTTTTAGTC
CAGAGTACTCAATGGTCTTTGTTCTTTTTTTTGTATGAAAATGCCTTTTT
ACCTTTTGCTATGGGTATTATTGCTATGTCTGCTTTTGCAATGATGTTTG
TCAAACATAAGCATGCATTTCTCTGTTTGTTTTTGTTACCTTCTCTTGCC
ACTGTAGCTTATTTTAATATGGTCTATATGCCTGCTAGTTGGGTGATGCG
TATTATGACATGGTTGGATATGGTTGATACTAGTTTGTCTGGTTTTAAGC
TAAAAGACTGTGTTATGTATGCATCAGCTGTAGTGTTACTAATCCTTATG
ACAGCAAGAACTGTGTATGATGATGGTGCTAGGAGAGTGTGGACACTTAT
GAATGTCTTGACACTCGTTTATAAAGTTTATTATGGTAATGCTTTAGATC
AAGCCATTTCCATGTGGGCTCTTATAATCTCTGTTACTTCTAACTACTCA
GGTGTAGTTACAACTGTCATGTTTTTGGCCAGAGGTATTGTTTTTATGTG
TGTTGAGTATTGCCCTATTTTCTTCATAACTGGTAATACACTTCAGTGTA
TAATGCTAGTTTATTGTTTCTTAGGCTATTTTTGTACTTGTTACTTTGGC
CTCTTTTGTTTACTCAACCGCTACTTTAGACTGACTCTTGGTGTTTATGA
TTACTTAGTTTCTACACAGGAGTTTAGATATATGAATTCACAGGGACTAC
TCCCACCCAAGAATAGCATAGATGCCTTCAAACTCAACATTAAATTGTTG
GGTGTTGGTGGCAAACCTTGTATCAAAGTAGCCACTGTACAGTCTAAAAT
GTCAGATGTAAAGTGCACATCAGTAGTCTTACTCTCAGTTTTGCAACAAC
TCAGAGTAGAATCATCATCTAAATTGTGGGCTCAATGTGTCCAGTTACAC
AATGACATTCTCTTAGCTAAAGATACTACTGAAGCCTTTGAAAAAATGGT
TTCACTACTTTCTGTTTTGCTTTCCATGCAGGGTGCTGTAGACATAAACA
AGCTTTGTGAAGAAATGCTGGACAACAGGGCAACCTTACAAGCTATAGCC
TCAGAGTTTAGTTCCCTTCCATCATATGCAGCTTTTGCTACTGCTCAAGA
AGCTTATGAGCAGGCTGTTGCTAATGGTGATTCTGAAGTTGTTCTTAAAA
AGTTGAAGAAGTCTTTGAATGTGGCTAAATCTGAATTTGACCGTGATGCA
GCCATGCAACGTAAGTTGGAAAAGATGGCTGATCAAGCTATGACCCAAAT
GTATAAACAGGCTAGATCTGAGGACAAGAGGGCAAAAGTTACTAGTGCTA
TGCAGACAATGCTTTTCACTATGCTTAGAAAGTTGGATAATGATGCACTC
AACAACATTATCAACAATGCAAGAGATGGTTGTGTTCCCTTGAACATAAT
ACCTCTTACAACAGCAGCCAAACTAATGGTTGTCATACCAGACTATAACA
CATATAAAAATACGTGTGATGGTACAACATTTACTTATGCATCAGCATTG
TGGGAAATCCAACAGGTTGTAGATGCAGATAGTAAAATTGTTCAACTTAG
TGAAATTAGTATGGACAATTCACCTAATTTAGCATGGCCTCTTATTGTAA
CAGCTTTAAGGGCCAATTCTGCTGTCAAATTACAGAATAATGAGCTTAGT
CCTGTTGCACTACGACAGATGTCTTGTGCTGCCGGTACTACACAAACTGC
TTGCACTGATGACAATGCGTTAGCTTACTACAACACAACAAAGGGAGGTA
GGTTTGTACTTGCACTGTTATCCGATTTACAGGATTTGAAATGGGCTAGA
TTCCCTAAGAGTGATGGAACTGGTACTATCTATACAGAACTGGAACCACC
TTGTAGGTTTGTTACAGACACACCTAAAGGTCCTAAAGTGAAGTATTTAT
ACTTTATTAAAGGATTAAACAACCTAAATAGAGGTATGGTACTTGGTAGT
TTAGCTGCCACAGTACGTCTACAAGCTGGTAATGCAACAGAAGTGCCTGC
CAATTCAACTGTATTATCTTTCTGTGCTTTTGCTGTAGATGCTGCTAAAG
CTTACAAAGATTATCTAGCTAGTGGGGGACAACCAATCACTAATTGTGTT
AAGATGTTGTGTACACACACTGGTACTGGTCAGGCAATAACAGTTACACC
GGAAGCCAATATGGATCAAGAATCCTTTGGTGGTGCATCGTGTTGTCTGT
ACTGCCGTTGCCACATAGATCATCCAAATCCTAAAGGATTTTGTGACTTA
AAAGGTAAGTATGTACAAATACCTACAACTTGTGCTAATGACCCTGTGGG
TTTTACACTTAAAAACACAGTCTGTACCGTCTGCGGTATGTGGAAAGGTT
ATGGCTGTAGTTGTGATCAACTCCGCGAACCCATGCTTCAGTCAGCTGAT
GCACAATCGTTTTTAAACGGGTTTGCGGTGTAAGTGCAGCCCGTCTTACA
CCGTGCGGCACAGGCACTAGTACTGATGTCGTATACAGGGCTTTTGACAT
CTACAATGATAAAGTAGCTGGTTTTGCTAAATTCCTAAAAACTAATTGTT
GTCGCTTCCAAGAAAAGGACGAAGATGACAATTTAATTGATTCTTACTTT
GTAGTTAAGAGACACACTTTCTCTAACTACCAACATGAAGAAACAATTTA
TAATTTACTTAAGGATTGTCCAGCTGTTGCTAAACATGACTTCTTTAAGT
TTAGAATAGACGGTGACATGGTACCACATATATCACGTCAACGTCTTACT
AAATACACAATGGCAGACCTCGTCTATGCTTTAAGGCATTTTGATGAAGG
TAATTGTGACACATTAAAAGAAATACTTGTCACATACAATTGTTGTGATG
ATGATTATTTCAATAAAAAGGACTGGTATGATTTTGTAGAAAACCCAGAT
ATATTACGCGTATACGCCAACTTAGGTGAACGTGTACGCCAAGCTTTGTT
AAAAACAGTACAATTCTGTGATGCCATGCGAAATGCTGGTATTGTTGGTG
TACTGACATTAGATAATCAAGATCTCAATGGTAACTGGTATGATTTCGGT
GATTTCATACAAACCACGCCAGGTAGTGGAGTTCCTGTTGTAGATTCTTA
TTATTCATTGTTAATGCCTATATTAACCTTGACCAGGGCTTTAACTGCAG
AGTCACATGTTGACACTGACTTAACAAAGCCTTACATTAAGTGGGATTTG
TTAAAATATGACTTCACGGAAGAGAGGTTAAAACTCTTTGACCGTTATTT
TAAATATTGGGATCAGACATACCACCCAAATTGTGTTAACTGTTTGGATG
ACAGATGCATTCTGCATTGTGCAAACTTTAATGTTTTATTCTCTACAGTG
TTCCCACCTACAAGTTTTGGACCACTAGTGAGAAAAATATTTGTTGATGG
TGTTCCATTTGTAGTTTCAACTGGATACCACTTCAGAGAGCTAGGTGTTG
TACATAATCAGGATGTAAACTTACATAGCTCTAGACTTAGTTTTAAGGAA
TTACTTGTGTATGCTGCTGACCCTGCTATGCACGCTGCTTCTGGTAATCT
ATTACTAGATAAACGCACTACGTGCTTTTCAGTAGCTGCACTTACTAACA
ATGTTGCTTTTCAAACTGTCAAACCCGGTAATTTTAACAAAGACTTCTAT
GACTTTGCTGTGTCTAAGGGTTTCTTTAAGGAAGGAAGTTCTGTTGAATT
AAAACACTTCTTCTTTGCTCAGGATGGTAATGCTGCTATCAGCGATTATG
ACTACTATCGTTATAATCTACCAACAATGTGTGATATCAGACAACTACTA
TTTGTAGTTGAAGTTGTTGATAAGTACTTTGATTGTTACGATGGTGGCTG
TATTAATGCTAACCAAGTCATCGTCAACAACCTAGACAAATCAGCTGGTT
TTCCATTTAATAAATGGGGTAAGGCTAGACTTTATTATGATTCAATGAGT
TATGAGGATCAAGATGCACTTTTCGCATATACAAAACGTAATGTCATCCC
TACTATAACTCAAATGAATCTTAAGTATGCCATTAGTGCAAAGAATAGAG
CTCGCACCGTAGCTGGTGTCTCTATCTGTAGTACTATGACCAATAGACAG
TTTCATCAAAAATTATTGAAATCAATAGCCGCCACTAGAGGAGCTACTGT
AGTAATTGGAACAAGCAAATTCTATGGTGGTTGGCACAACATGTTAAAAA
CTGTTTATAGTGATGTAGAAAACCCTCACCTTATGGGTTGGGATTATCCT
AAATGTGATAGAGCCATGCCTAACATGCTTAGAATTATGGCCTCACTTGT
TCTTGCTCGCAAACATACAACGTGTTGTAGCTTGTCACACCGTTTCTATA
GATTAGCTAATGAGTGTGCTCAAGTATTGAGTGAAATGGTCATGTGTGGC
GGTTCACTATATGTTAAACCAGGTGGAACCTCATCAGGAGATGCCACAAC
TGCTTATGCTAATAGTGTTTTTAACATTTGTCAAGCTGTCACGGCCAATG
TTAATGCACTTTTATCTACTGATGGTAACAAAATTGCCGATAAGTATGTC
CGCAATTTACAACACAGACTTTATGAGTGTCTCTATAGAAATAGAGATGT
TGACACAGACTTTGTGAATGAGTTTTACGCATATTTGCGTAAACATTTCT
CAATGATGATACTCTCTGACGATGCTGTTGTGTGTTTCAATAGCACTTAT
GCATCTCAAGGTCTAGTGGCTAGCATAAAGAACTTTAAGTCAGTTCTTTA
TTATCAAAACAATGTTTTTATGTCTGAAGCAAAATGTTGGACTGAGACTG
ACCTTACTAAAGGACCTCATGAATTTTGCTCTCAACATACAATGCTAGTT
AAACAGGGTGATGATTATGTGTACCTTCCTTACCCAGATCCATCAAGAAT
CCTAGGGGCCGGCTGTTTTGTAGATGATATCGTAAAAACAGATGGTACAC
TTATGATTGAACGGTTCGTGTCTTTAGCTATAGATGCTTACCCACTTACT
AAACATCCTAATCAGGAGTATGCTGATGTCTTTCATTTGTACTTACAATA
CATAAGAAAGCTACATGATGAGTTAACAGGACACATGTTAGACATGTATT
CTGTTATGCTTACTAATGATAACACTTCAAGGTATTGGGAACCTGAGTTT
TATGAGGCTATGTACACACCGCATACAGTCTTACAGGCTGTTGGGGCTTG
TGTTCTTTGCAATTCACAGACTTCATTAAGATGTGGTGCTTGCATACGTA
GACCATTCTTATGTTGTAAATGCTGTTACGACCATGTCATATCAACATCA
CATAAATTAGTCTTGTCTGTTAATCCGTATGTTTGCAATGCTCCAGGTTG
TGATGTCACAGATGTGACTCAACTTTACTTAGGAGGTATGAGCTATTATT
GTAAATCACATAAACCACCCATTAGTTTTCCATTGTGTGCTAATGGACAA
GTTTTTGGTTTATATAAAAATACATGTGTTGGTAGCGATAATGTTACTGA
CTTTAATGCAATTGCAACATGTGACTGGACAAATGCTGGTGATTACATTT
TAGCTAACACCTGTACTGAAAGACTCAAGCTTTTTGCAGCAGAAACGCTC
AAAGCTACTGAGGAGACATTTAAACTGTCTTATGGTATTGCTACTGTACG
TGAAGTGCTGTCTGACAGAGAATTACATCTTTCATGGGAAGTTGGTAAAC
CTAGACCACCACTTAACCGAAATTATGTCTTTACTGGTTATCGTGTAACT
AAAAACAGTAAAGTACAAATAGGAGAGTACACCTTTGAAAAAGGTGACTA
TGGTGATGCTGTTGTTTACCGAGGTACAACAACTTACAAATTAAATGTTG
GTGATTATTTTGTGCTGACATCACATACAGTAATGCCATTAAGTGCACCT
ACACTAGTGCCACAAGAGCACTATGTTAGAATTACTGGCTTATACCCAAC
ACTCAATATCTCAGATGAGTTTTCTAGCAATGTTGCAAATTATCAAAAGG
TTGGTATGCAAAAGTATTCTACACTCCAGGGACCACCTGGTACTGGTAAG
AGTCATTTTGCTATTGGCCTAGCTCTCTACTACCCTTCTGCTCGCATAGT
GTATACAGCTTGCTCTCATGCCGCTGTTGATGCACTATGTGAGAAGGCAT
TAAAATATTTGCCTATAGATAAATGTAGTAGAATTATACCTGCACGTGCT
CGTGTAGAGTGTTTTGATAAATTCAAAGTGAATTCAACATTAGAACAGTA
TGTCTTTTGTACTGTAAATGCATTGCCTGAGACGACAGCAGATATAGTTG
TCTTTGATGAAATTTCAATGGCCACAAATTATGATTTGAGTGTTGTCAAT
GCCAGATTACGTGCTAAGCACTATGTGTACATTGGCGACCCTGCTCAATT
ACCTGCACCACGCACATTGCTAACTAAGGGCACACTAGAACCAGAATATT
TCAATTCAGTGTGTAGACTTATGAAAACTATAGGTCCAGACATGTTCCTC
GGAACTTGTCGGCGTTGTCCTGCTGAAATTGTTGACACTGTGAGTGCTTT
GGTTTATGATAATAAGCTTAAAGCACATAAAGACAAATCAGCTCAATGCT
TTAAAATGTTTTATAAGGGTGTTATCACGCATGATGTTTCATCTGCAATT
AACAGGCCACAAATAGGCGTGGTAAGAGAATTCCTTACACGTAACCCTGC
TTGGAGAAAAGCTGTCTTTATTTCACCTTATAATTCACAGAATGCTGTAG
CCTCAAAGATTTTGGGACTACCAACTCAAACTGTTGATTCATCACAGGGC
TCAGAATATGACTATGTCATATTCACTCAAACCACTGAAACAGCTCACTC
TTGTAATGTAAACAGATTTAATGTTGCTATTACCAGAGCAAAAGTAGGCA
TACTTTGCATAATGTCTGATAGAGACCTTTATGACAAGTTGCAATTTACA
AGTCTTGAAATTCCACGTAGGAATGTGGCAACTTTACAAGCTGAAAATGT
AACAGGACTCTTTAAAGATTGTAGTAAGGTAATCACTGGGTTACATCCTA
CACAGGCACCTACACACCTCAGTGTTGACACTAAATTCAAAACTGAAGGT
TTATGTGTTGACATACCTGGCATACCTAAGGACATGACCTATAGAAGACT
CATCTCTATGATGGGTTTTAAAATGAATTATCAAGTTAATGGTTACCCTA
ACATGTTTATCACCCGCGAAGAAGCTATAAGACATGTACGTGCATGGATT
GGCTTCGATGTCGAGGGGTGTCATGCTACTAGAGAAGCTGTTGGTACCAA
TTTACCTTTACAGCTAGGTTTTTCTACAGGTGTTAACCTAGTTGCTGTAC
CTACAGGTTATGTTGATACACCTAATAATACAGATTTTTCCAGAGTTAGT
GCTAAACCACCGCCTGGAGATCAATTTAAACACCTCATACCACTTATGTA
CAAAGGACTTCCTTGGAATGTAGTGCGTATAAAGATTGTACAAATGTTAA
GTGACACACTTAAAAATCTCTCTGACAGAGTCGTATTTGTCTTATGGGCA
CATGGCTTTGAGTTGACATCTATGAAGTATTTTGTGAAAATAGGACCTGA
GCGCACCTGTTGTCTATGTGATAGACGTGCCACATGCTTTTCCACTGCTT
CAGACACTTATGCCTGTTGGCATCATTCTATTGGATTTGATTACGTCTAT
AATCCGTTTATGATTGATGTTCAACAATGGGGTTTTACAGGTAACCTACA
AAGCAACCATGATCTGTATTGTCAAGTCCATGGTAATGCACATGTAGCTA
GTTGTGATGCAATCATGACTAGGTGTCTAGCTGTCCACGAGTGCTTTGTT
AAGCGTGTTGACTGGACTATTGAATATCCTATAATTGGTGATGAACTGAA
GATTAATGCGGCTTGTAGAAAGGTTCAACACATGGTTGTTAAAGCTGCAT
TATTAGCAGACAAATTCCCAGTTCTTCACGACATTGGTAACCCTAAAGCT
ATTAAGTGTGTACCTCAAGCTGATGTAGAATGGAAGTTCTATGATGCACA
GCCTTGTAGTGACAAAGCTTATAAAATAGAAGAATTATTCTATTCTTATG
CCACACATTCTGACAAATTCACAGATGGTGTATGCCTATTTTGGAATTGC
AATGTCGATAGATATCCTGCTAATTCCATTGTTTGTAGATTTGACACTAG
AGTGCTATCTAACCTTAACTTGCCTGGTTGTGATGGTGGCAGTTTGTATG
TAAATAAACATGCATTCCACACACCAGCTTTTGATAAAAGTGCTTTTGTT
AATTTAAAACAATTACCATTTTTCTATTACTCTGACAGTCCATGTGAGTC
TCATGGAAAACAAGTAGTGTCAGATATAGATTATGTACCACTAAAGTCTG
CTACGTGTATAACACGTTGCAATTTAGGTGGTGCTGTCTGTAGACATCAT
GCTAATGAGTACAGATTGTATCTCGATGCTTATAACATGATGATCTCAGC
TGGCTTTAGCTTGTGGGTTTACAAACAATTTGATACTTATAACCTCTGGA
ACACTTTTACAAGACTTCAGAGTTTAGAAAATGTGGCTTTTAATGTTGTA
AATAAGGGACACTTTGATGGACAACAGGGTGAAGTACCAGTTTCTATCAT
TAATAACACTGTTTACACAAAAGTTGATGGTGTTGATGTAGAATTGTTTG
AAAATAAAACAACATTACCTGTTAATGTAGCATTTGAGCTTTGGGCTAAG
CGCAACATTAAACCAGTACCAGAGGTGAAAATACTCAATAATTTGGGTGT
GGACATTGCTGCTAATACTGTGATCTGGGACTACAAAAGAGATGCTCCAG
CACATATATCTACTATTGGTGTTTGTTCTATGACTGACATAGCCAAGAAA
CCAACTGAAACGATTTGTGCACCACTCACTGTCTTTTTTGATGGTAGAGT
TGATGGTCAAGTAGACTTATTTAGAAATGCCCGTAATGGTGTTCTTATTA
CAGAAGGTAGTGTTAAAGGTTTACAACCATCTGTAGGTCCCAAACAAGCT
AGTCTTAATGGAGTCACATTAATTGGAGAAGCCGTAAAAACACAGTTCAA
TTATTATAAGAAAGTTGATGGTGTTGTCCAACAATTACCTGAAACTTACT
TTACTCAGAGTAGAAATTTACAAGAATTTAAACCCAGGAGTCAAATGGAA
ATTGATTTCTTAGAATTAGCTATGGATGAATTCATTGAACGGTATAAATT
AGAAGGCTATGCCTTCGAACATATCGTTTATGGAGATTTTAGTCATAGTC
AGTTAGGTGGTTTACATCTACTGATTGGACTAGCTAAACGTTTTAAGGAA
TCACCTTTTGAATTAGAAGATTTTATTCCTATGGACAGTACAGTTAAAAA
CTATTTCATAACAGATGCGCAAACAGGTTCATCTAAGTGTGTGTGTTCTG
TTATTGATTTATTACTTGATGATTTTGTTGAAATAATAAAATCCCAAGAT
TTATCTGTAGTTTCTAAGGTTGTCAAAGTGACTATTGACTATACAGAAAT
TTCATTTATGCTTTGGTGTAAAGATGGCCATGTAGAAACATTTTACCCAA
AATTACAATCTAGTCAAGCGTGGCAACCGGGTGTTGCTATGCCTAATCTT
TACAAAATGCAAAGAATGCTATTAGAAAAGTGTGACCTTCAAAATTATGG
TGATAGTGCAACATTACCTAAAGGCATAATGATGAATGTCGCAAAATATA
CTCAACTGTGTCAATATTTAAACACATTAACATTAGCTGTACCCTATAAT
ATGAGAGTTATACATTTTGGTGCTGGTTCTGATAAAGGAGTTGCACCAGG
TACAGCTGTTTTAAGACAGTGGTTGCCTACGGGTACGCTGCTTGTCGATT
CAGATCTTAATGACTTTGTCTCTGATGCAGATTCAACTTTGATTGGTGAT
TGTGCAACTGTACATACAGCTAATAAATGGGATCTCATTATTAGTGATAT
GTACGACCCTAAGACTAAAAATGTTACAAAAGAAAATGACTCTAAAGAGG
GTTTTTTCACTTACATTTGTGGGTTTATACAACAAAAGCTAGCTCTTGGA
GGTTCCGTGGCTATAAAGATAACAGAACATTCTTGGAATGCTGATCTTTA
TAAGCTCATGGGACACTTCGCATGGTGGACAGCCTTTGTTACTAATGTGA
ATGCGTCATCATCTGAAGCATTTTTAATTGGATGTAATTATCTTGGCAAA
CCACGCGAACAAATAGATGGTTATGTCATGCATGCAAATTACATATTTTG
GAGGAATACAAATCCAATTCAGTTGTCTTCCTATTCTTTATTTGACATGA
GTAAATTTCCCCTTAAATTAAGGGGTACTGCTGTTATGTCTTTAAAAGAA
GGTCAAATCAATGATATGATTTTATCTCTTCTTAGTAAAGGTAGACTTAT
AATTAGAGAAAACAACAGAGTTGTTATTTCTAGTGATGTTCTTGTTAACA
ACTAAACGAACAATGTTTGTTTTTCTTGTTTTATTGCCACTAGTCTCTAG
TCAGTGTGTTAATCTTACAACCAGAACTCAATTACCCCCTGCATACACTA
ATTCTTTCACACGTGGTGTTTATTACCCTGACAAAGTTTTCAGATCCTCA
GTTTTACATTCAACTCAGGACTTGTTCTTACCTTTCTTTTCCAATGTTAC
TTGGTTCCATGCTATACATGTCTCTGGGACCAATGGTACTAAGAGGTTTG
ATAACCCTGTCCTACCATTTAATGATGGTGTTTATTTTGCTTCCACTGAG
AAGTCTAACATAATAAGAGGCTGGATTTTTGGTACTACTTTAGATTCGAA
GACCCAGTCCCTACTTATTGTTAATAACGCTACTAATGTTGTTATTAAAG
TCTGTGAATTTCAATTTTGTAATGATCCATTTTTGGGTGTTTATTACCAC
AAAAACAACAAAAGTTGGATGGAAAGTGAGTTCAGAGTTTATTCTAGTGC
GAATAATTGCACTTTTGAATATGTCTCTCAGCCTTTTCTTATGGACCTTG
AAGGAAAACAGGGTAATTTCAAAAATCTTAGGGAATTTGTGTTTAAGAAT
ATTGATGGTTATTTTAAAATATATTCTAAGCACACGCCTATTAATTTAGT
GCGTGATCTCCCTCAGGGTTTTTCGGCTTTAGAACCATTGGTAGATTTGC
CAATAGGTATTAACATCACTAGGTTTCAAACTTTACTTGCTTTACATAGA
AGTTATTTGACTCCTGGTGATTCTTCTTCAGGTTGGACAGCTGGTGCTGC
AGCTTATTATGTGGGTTATCTTCAACCTAGGACTTTTCTATTAAAATATA
ATGAAAATGGAACCATTACAGATGCTGTAGACTGTGCACTTGACCCTCTC
TCAGAAACAAAGTGTACGTTGAAATCCTTCACTGTAGAAAAAGGAATCTA
TCAAACTTCTAACTTTAGAGTCCAACCAACAGAATCTATTGTTAGATTTC
CTAATATTACAAACTTGTGCCCTTTTGGTGAAGTTTTTAACGCCACCAGA
TTTGCATCTGTTTATGCTTGGAACAGGAAGAGAATCAGCAACTGTGTTGC
TGATTATTCTGTCCTATATAATTCCGCATCATTTTCCACTTTTAAGTGTT
ATGGAGTGTCTCCTACTAAATTAAATGATCTCTGCTTTACTAATGTCTAT
GCAGATTCATTTGTAATTAGAGGTGATGAAGTCAGACAAATCGCTCCAGG
GCAAACTGGAAAGATTGCTGATTATAATTATAAATTACCAGATGATTTTA
CAGGCTGCGTTATAGCTTGGAATTCTAACAATCTTGATTCTAAGGTTGGT
GGTAATTATAATTACCTGTATAGATTGTTTAGGAAGTCTAATCTCAAACC
TTTTGAGAGAGATATTTCAACTGAAATCTATCAGGCCGGTAGCACACCTT
GTAATGGTGTTGAAGGTTTTAATTGTTACTTTCCTTTACAATCATATGGT
TTCCAACCCACTAATGGTGTTGGTTACCAACCATACAGAGTAGTAGTACT
TTCTTTTGAACTTCTACATGCACCAGCAACTGTTTGTGGACCTAAAAAGT
CTACTAATTTGGTTAAAAACAAATGTGTCAATTTCAACTTCAATGGTTTA
ACAGGCACAGGTGTTCTTACTGAGTCTAACAAAAAGTTTCTGCCTTTCCA
ACAATTTGGCAGAGACATTGCTGACACTACTGATGCTGTCCGTGATCCAC
AGACACTTGAGATTCTTGACATTACACCATGTTCTTTTGGTGGTGTCAGT
GTTATAACACCAGGAACAAATACTTCTAACCAGGTTGCTGTTCTTTATCA
GGATGTTAACTGCACAGAAGTCCCTGTTGCTATTCATGCAGATCAACTTA
CTCCTACTTGGCGTGTTTATTCTACAGGTTCTAATGTTTTTCAAACACGT
GCAGGCTGTTTAATAGGGGCTGAACATGTCAACAACTCATATGAGTGTGA
CATACCCATTGGTGCAGGTATATGCGCTAGTTATCAGACTCAGACTAATT
CTCCTCGGCGGGCACGTAGTGTAGCTAGTCAATCCATCATTGCCTACACT
ATGTCACTTGGTGCAGAAAATTCAGTTGCTTACTCTAATAACTCTATTGC
CATACCCACAAATTTTACTATTAGTGTTACCACAGAAATTCTACCAGTGT
CTATGACCAAGACATCAGTAGATTGTACAATGTACATTTGTGGTGATTCA
ACTGAATGCAGCAATCTTTTGTTGCAATATGGCAGTTTTTGTACACAATT
AAACCGTGCTTTAACTGGAATAGCTGTTGAACAAGACAAAAACACCCAAG
AAGTTTTTGCACAAGTCAAACAAATTTACAAAACACCACCAATTAAAGAT
TTTGGTGGTTTTAATTTTTCACAAATATTACCAGATCCATCAAAACCAAG
CAAGAGGTCATTTATTGAAGATCTACTTTTCAACAAAGTGACACTTGCAG
ATGCTGGCTTCATCAAACAATATGGTGATTGCCTTGGTGATATTGCTGCT
AGAGACCTCATTTGTGCACAAAAGTTTAACGGCCTTACTGTTTTGCCACC
TTTGCTCACAGATGAAATGATTGCTCAATACACTTCTGCACTGTTAGCGG
GTACAATCACTTCTGGTTGGACCTTTGGTGCAGGTGCTGCATTACAAATA
CCATTTGCTATGCAAATGGCTTATAGGTTTAATGGTATTGGAGTTACACA
GAATGTTCTCTATGAGAACCAAAAATTGATTGCCAACCAATTTAATAGTG
CTATTGGCAAAATTCAAGACTCACTTTCTTCCACAGCAAGTGCACTTGGA
AAACTTCAAGATGTGGTCAACCAAAATGCACAAGCTTTAAACACGCTTGT
TAAACAACTTAGCTCCAATTTTGGTGCAATTTCAAGTGTTTTAAATGATA
TCCTTTCACGTCTTGACAAAGTTGAGGCTGAAGTGCAAATTGATAGGTTG
ATCACAGGCAGACTTCAAAGTTTGCAGACATATGTGACTCAACAATTAAT
TAGAGCTGCAGAAATCAGAGCTTCTGCTAATCTTGCTGCTACTAAAATGT
CAGAGTGTGTACTTGGACAATCAAAAAGAGTTGATTTTTGTGGAAAGGGC
TATCATCTTATGTCCTTCCCTCAGTCAGCACCTCATGGTGTAGTCTTCTT
GCATGTGACTTATGTCCCTGCACAAGAAAAGAACTTCACAACTGCTCCTG
CCATTTGTCATGATGGAAAAGCACACTTTCCTCGTGAAGGTGTCTTTGTT
TCAAATGGCACACACTGGTTTGTAACACAAAGGAATTTTTATGAACCACA
AATCATTACTACAGACAACACATTTGTGTCTGGTAACTGTGATGTTGTAA
TAGGAATTGTCAACAACACAGTTTATGATCCTTTGCAACCTGAATTAGAC
TCATTCAAGGAGGAGTTAGATAAATATTTTAAGAATCATACATCACCAGA
TGTTGATTTAGGTGACATCTCTGGCATTAATGCTTCAGTTGTAAACATTC
AAAAAGAAATTGACCGCCTCAATGAGGTTGCCAAGAATTTAAATGAATCT
CTCATCGATCTCCAAGAACTTGGAAAGTATGAGCAGTATATAAAATGGCC
ATGGTACATTTGGCTAGGTTTTATAGCTGGCTTGATTGCCATAGTAATGG
TGACAATTATGCTTTGCTGTATGACCAGTTGCTGTAGTTGTCTCAAGGGC
TGTTGTTCTTGTGGATCCTGCTGCAAATTTGATGAAGACGACTCTGAGCC
AGTGCTCAAAGGAGTCAAATTACATTACACATAAACGAACTTATGGATTT
GTTTATGAGAATCTTCACAATTGGAACTGTAACTTTGAAGCAAGGTGAAA
TCAAGGATGCTACTCCTTCAGATTTTGTTCGCGCTACTGCAACGATACCG
ATACAAGCCTCACTCCCTTTCGGATGGCTTATTGTTGGCGTTGCACTTCT
TGCTGTTTTTCAGAGCGCTTCCAAAATCATAACCCTCAAAAAGAGATGGC
AACTAGCACTCTCCAAGGGTGTTCACTTTGTTTGCAACTTGCTGTTGTTG
TTTGTAACAGTTTACTCACACCTTTTGCTCGTTGCTGCTGGCCTTGAAGC
CCCTTTTCTCTATCTTTATGCTTTAGTCTACTTCTTGCAGAGTATAAACT
TTGTAAGAATAATAATGAGGCTTTGGCTTTGCTGGAAATGCCGTTCCAAA
AACCCATTACTTTATGATGCCAACTATTTTCTTTGCTGGCATACTAATTG
TTACGACTATTGTATACCTTACAATAGTGTAACTTCTTCAATTGTCATTA
CTTCAGGTGATGGCACAACAAGTCCTATTTCTGAACATGACTACCAGATT
GGTGGTTATACTGAAAAATGGGAATCTGGAGTAAAAGACTGTGTTGTATT
ACACAGTTACTTCACTTCAGACTATTACCAGCTGTACTCAACTCAATTGA
GTACAGACACTGGTGTTGAACATGTTACCTTCTTCATCTACAATAAAATT
GTTGATGAGCCTGAAGAACATGTCCAAATTCACACAATCGACGGTTCATC
CGGAGTTGTTAATCCAGTAATGGAACCAATTTATGATGAACCGACGACGA
CTACTAGCGTGCCTTTGTAAGCACAAGCTGATGAGTACGAACTTATGTAC
TCATTCGTTTCGGAAGAGACAGGTACGTTAATAGTTAATAGCGTACTTCT
TTTTCTTGCTTTCGTGGTATTCTTGCTAGTTACACTAGCCATCCTTACTG
CGCTTCGATTGTGTGCGTACTGCTGCAATATTGTTAACGTGAGTCTTGTA
AAACCTTCTTTTTACGTTTACTCTCGTGTTAAAAATCTGAATTCTTCTAG
AGTTCCTGATCTTCTGGTCTAAACGAACTAAATATTATATTAGTTTTTCT
GTTTGGAACTTTAATTTTAGCCATGGCAGATTCCAACGGTACTATTACCG
TTGAAGAGCTTAAAAAGCTCCTTGAACAATGGAACCTAGTAATAGGTTTC
CTATTCCTTACATGGATTTGTCTTCTACAATTTGCCTATGCCAACAGGAA
TAGGTTTTTGTATATAATTAAGTTAATTTTCCTCTGGCTGTTATGGCCAG
TAACTTTAGCTTGTTTTGTGCTTGCTGCTGTTTACAGAATAAATTGGATC
ACCGGTGGAATTGCTATCGCAATGGCTTGTCTTGTAGGCTTGATGTGGCT
CAGCTACTTCATTGCTTCTTTCAGACTGTTTGCGCGTACGCGTTCCATGT
GGTCATTCAATCCAGAAACTAACATTCTTCTCAACGTGCCACTCCATGGC
ACTATTCTGACCAGACCGCTTCTAGAAAGTGAACTCGTAATCGGAGCTGT
GATCCTTCGTGGACATCTTCGTATTGCTGGACACCATCTAGGACGCTGTG
ACATCAAGGACCTGCCTAAAGAAATCACTGTTGCTACATCACGAACGCTT
TCTTATTACAAATTGGGAGCTTCGCAGCGTGTAGCAGGTGACTCAGGTTT
TGCTGCATACAGTCGCTACAGGATTGGCAACTATAAATTAAACACAGACC
ATTCCAGTAGCAGTGACAATATTGCTTTGCTTGTACAGTAAGTGACAACA
GATGTTTCATCTCGTTGACTTTCAGGTTACTATAGCAGAGATATTACTAA
TTATTATGAGGACTTTTAAAGTTTCCATTTGGAATCTTGATTACATCATA
AACCTCATAATTAAAAATTTATCTAAGTCACTAACTGAGAATAAATATTC
TCAATTAGATGAAGAGCAACCAATGGAGATTGATTAAACGAACATGAAAA
TTATTCTTTTCTTGGCACTGATAACACTCGCTACTTGTGAGCTTTATCAC
TACCAAGAGTGTGTTAGAGGTACAACAGTACTTTTAAAAGAACCTTGCTC
TTCTGGAACATACGAGGGCAATTCACCATTTCATCCTCTAGCTGATAACA
AATTTGCACTGACTTGCTTTAGCACTCAATTTGCTTTTGCTTGTCCTGAC
GGCGTAAAACACGTCTATCAGTTACGTGCCAGATCAGTTTCACCTAAACT
GTTCATCAGACAAGAGGAAGTTCAAGAACTTTACTCTCCAATTTTTCTTA
TTGTTGCGGCAATAGTGTTTATAACACTTTGCTTCACACTCAAAAGAAAG
ACAGAATGATTGAACTTTCATTAATTGACTTCTATTTGTGCTTTTTAGCC
TTTCTGCTATTCCTTGTTTTAATTATGCTTATTATCTTTTGGTTCTCACT
TGAACTGCAAGATCATAATGAAACTTGTCACGCCTAAACGAACATGAAAT
TTCTTGTTTTCTTAGGAATCATCACAACTGTAGCTGCATTTCACCAAGAA
TGTAGTTTACAGTCATGTACTCAACATCAACCATATGTAGTTGATGACCC
GTGTCCTATTCACTTCTATTCTAAATGGTATATTAGAGTAGGAGCTAGAA
AATCAGCACCTTTAATTGAATTGTGCGTGGATGAGGCTGGTTCTAAATCA
CCCATTCAGTACATCGATATCGGTAATTATACAGTTTCCTGTTTACCTTT
TACAATTAATTGCCAGGAACCTAAATTGGGTAGTCTTGTAGTGCGTTGTT
CGTTCTATGAAGACTTTTTAGAGTATCATGACGTTCGTGTTGTTTTAGAT
TTCATCTAAACGAACAAACTAAAATGTCTGATAATGGACCCCAAAATCAG
CGAAATGCACCCCGCATTACGTTTGGTGGACCCTCAGATTCAACTGGCAG
TAACCAGAATGGAGAACGCAGTGGGGCGCGATCAAAACAACGTCGGCCCC
AAGGTTTACCCAATAATACTGCGTCTTGGTTCACCGCTCTCACTCAACAT
GGCAAGGAAGACCTTAAATTCCCTCGAGGACAAGGCGTTCCAATTAACAC
CAATAGCAGTCCAGATGACCAAATTGGCTACTACCGAAGAGCTACCAGAC
GAATTCGTGGTGGTGACGGTAAAATGAAAGATCTCAGTCCAAGATGGTAT
TTCTACTACCTAGGAACTGGGCCAGAAGCTGGACTTCCCTATGGTGCTAA
CAAAGACGGCATCATATGGGTTGCAACTGAGGGAGCCTTGAATACACCAA
AAGATCACATTGGCACCCGCAATCCTGCTAACAATGCTGCAATCGTGCTA
CAACTTCCTCAAGGAACAACATTGCCAAAAGGCTTCTACGCAGAAGGGAG
CAGAGGCGGCAGTCAAGCCTCTTCTCGTTCCTCATCACGTAGTCGCAACA
GTTCAAGAAATTCAACTCCAGGCAGCAGTAGGGGAACTTCTCCTGCTAGA
ATGGCTGGCAATGGCGGTGATGCTGCTCTTGCTTTGCTGCTGCTTGACAG
ATTGAACCAGCTTGAGAGCAAAATGTCTGGTAAAGGCCAACAACAACAAG
GCCAAACTGTCACTAAGAAATCTGCTGCTGAGGCTTCTAAGAAGCCTCGG
CAAAAACGTACTGCCACTAAAGCATACAATGTAACACAAGCTTTCGGCAG
ACGTGGTCCAGAACAAACCCAAGGAAATTTTGGGGACCAGGAACTAATCA
GACAAGGAACTGATTACAAACATTGGCCGCAAATTGCACAATTTGCCCCC
AGCGCTTCAGCGTTCTTCGGAATGTCGCGCATTGGCATGGAAGTCACACC
TTCGGGAACGTGGTTGACCTACACAGGTGCCATCAAATTGGATGACAAAG
ATCCAAATTTCAAAGATCAAGTCATTTTGCTGAATAAGCATATTGACGCA
TACAAAACATTCCCACCAACAGAGCCTAAAAAGGACAAAAAGAAGAAGGC
TGATGAAACTCAAGCCTTACCGCAGAGACAGAAGAAACAGCAAACTGTGA
CTCTTCTTCCTGCTGCAGATTTGGATGATTTCTCCAAACAATTGCAACAA
TCCATGAGCAGTGCTGACTCAACTCAGGCCTAAACTCATGCAGACCACAC
AAGGCAGATGGGCTATATAAACGTTTTCGCTTTTCCGTTTACGATATATA
GTCTACTCTTGTGCAGAATGAATTCTCGTAACTACATAGCACAAGTAGAT
GTAGTTAACTTTAATCTCACATAGCAATCTTTAATCAGTGTGTAACATTA
GGGAGGACTTGAAAGAGCCACCACATTTTCACCGAGGCCACGCGGAGTAC
GATCGAGTGTACAGTGAACAATGCTAGGGAGAGCTGCCTATATGGAAGAG
CCCTAATGTGTAAAATTAATTTTAGTAGTGCTATCCCCATGTGATTTTAA
TAGCTTCTTAGGAGAATGACAAAAAAAAAAAAAAAAAAAAAAAAAAAAAA
AAA (SEQ ID NO: 1)

TABLE 5
SARS-CoV2 gene sequences

| Gene | Start | End | Sequence |
|---|---|---|---|
| orf1a | 266 | 13483 | ATGGAGAGCCTTGTCCCTGGTTTCAACGAGAAAACAC |
| (SEQ ID | | | ACGTCCAACTCAGTTTGCCTGTTTTACAGGTTCGCGAC |
| NO: 2) | | | GTGCTCGTACGTGGCTTTGGAGACTCCGTGGAGGAGG |
| | | | TCTTATCAGAGGCACGTCAACATCTTAAAGATGGCAC |
| | | | TTGTGGCTTAGTAGAAGTTGAAAAAGGCGTTTTGCCTC |
| | | | AACTTGAACAGCCCTATGTGTTCATCAAACGTTCGGAT |
| | | | GCTCGAACTGCACCTCATGGTCATGTTATGGTTGAGCT |
| | | | GGTAGCAGAACTCGAAGGCATTCAGTACGGTCGTAGT |
| | | | GGTGAGACACTTGGTGTCCTTGTCCCTCATGTGGGCGA |
| | | | AATACCAGTGGCTTACCGCAAGGTTCTTCTTCGTAAGA |
| | | | ACGGTAATAAAGGAGCTGGTGGCCATAGTTACGGCGC |
| | | | CGATCTAAAGTCATTTGACTTAGGCGACGAGCTTGGC |
| | | | ACTGATCCTTATGAAGATTTTCAAGAAAACTGGAACA |
| | | | CTAAACATAGCAGTGGTGTTACCCGTGAACTCATGCG |
| | | | TGAGCTTAACGGAGGGGCATACACTCGCTATGTCGAT |
| | | | AACAACTTCTGTGGCCCTGATGGCTACCCTCTTGAGTG |
| | | | CATTAAAGACCTTCTAGCACGTGCTGGTAAAGCTTCAT |
| | | | GCACTTTGTCCGAACAACTGGACTTTATTGACACTAAG |
| | | | AGGGGTGTATACTGCTGCCGTGAACATGAGCATGAAA |
| | | | TTGCTTGGTACACGGAACGTTCTGAAAAGAGCTATGA |
| | | | ATTGCAGACACCTTTTGAAATTAAATTGGCAAAGAAA |
| | | | TTTGACACCTTCAATGGGGAATGTCCAAATTTTGTATT |
| | | | TCCCTTAAATTCCATAATCAAGACTATTCAACCAAGGG |
| | | | TTGAAAAGAAAAAGCTTGATGGCTTTATGGGTAGAAT |
| | | | TCGATCTGTCTATCCAGTTGCGTCACCAAATGAATGCA |
| | | | ACCAAATGTGCCTTTCAACTCTCATGAAGTGTGATCAT |
| | | | TGTGGTGAAACTTCATGGCAGACGGGCGATTTTGTTA |
| | | | AAGCCACTTGCGAATTTTGTGGCACTGAGAATTTGACT |
| | | | AAAGAAGGTGCCACTACTTGTGGTTACTTACCCCAAA |
| | | | ATGCTGTTGTTAAAATTTATTGTCCAGCATGTCACAAT |
| | | | TCAGAAGTAGGACCTGAGCATAGTCTTGCCGAATACC |
| | | | ATAATGAATCTGGCTTGAAAACCATTCTTCGTAAGGGT |
| | | | GGTCGCACTATTGCCTTTGGAGGCTGTGTGTTCTCTTA |
| | | | TGTTGGTTGCCATAACAAGTGTGCCTATTGGGTTCCAC |
| | | | GTGCTAGCGCTAACATAGGTTGTAACCATACAGGTGT |
| | | | TGTTGGAGAAGGTTCCGAAGGTCTTAATGACAACCTT |
| | | | CTTGAAATACTCCAAAAAGAGAAAGTCAACATCAATA |
| | | | TTGTTGGTGACTTTAAACTTAATGAAGAGATCGCCATT |
| | | | ATTTTGGCATCTTTTTCTGCTTCCACAAGTGCTTTTGT |
| | | | GGAAACTGTGAAAGGTTTGGATTATAAAGCATTCAAAC |
| | | | AAATTGTTGAATCCTGTGGTAATTTTAAAGTTACAAAA |
| | | | GGAAAAGCTAAAAAAGGTGCCTGGAATATTGGTGAAC |
| | | | AGAAATCAATACTGAGTCCTCTTTATGCATTTGCATCA |
| | | | GAGGCTGCTCGTGTTGTACGATCAATTTTCTCCCGCAC |
| | | | TCTTGAAACTGCTCAAAATTCTGTGCGTGTTTTACAGA |
| | | | AGGCCGCTATAACAATACTAGATGGAATTTCACAGTA |
| | | | TTCACTGAGACTCATTGATGCTATGATGTTCACATCTG |
| | | | ATTTGGCTACTAACAATCTAGTTGTAATGGCCTACATT |
| | | | ACAGGTGGTGTTGTTCAGTTGACTTCGCAGTGGCTAAC |
| | | | TAACATCTTTGGCACTGTTTATGAAAAACTCAAACCCG |
| | | | TCCTTGATTGGCTTGAAGAGAAGTTTAAGGAAGGTGT |
| | | | AGAGTTTCTTAGAGACGGTTGGGAAATTGTTAAATTTA |
| | | | TCTCAACCTGTGCTTGTGAAATTGTCGGTGGACAAATT |
| | | | GTCACCTGTGCAAAGGAAATTAAGGAGAGTGTTCAGA |
| | | | CATTCTTTAAGCTTGTAAATAAATTTTTGGCTTTGTGT |
| | | | GCTGACTCTATCATTATTGGTGGAGCTAAACTTAAAGC |
| | | | CTTGAATTTAGGTGAAACATTTGTCACGCACTCAAAG |
| | | | GGATTGTACAGAAAGTGTGTTAAATCCAGAGAAGAAA |
| | | | CTGGCCTACTCATGCCTCTAAAAGCCCCAAAAGAAAT |
| | | | TATCTTCTTAGAGGGAGAAACACTTCCCACAGAAGTG |
| | | | TTAACAGAGGAAGTTGTCTTGAAAACTGGTGATTTAC |
| | | | AACCATTAGAACAACCTACTAGTGAAGCTGTTGAAGC |
| | | | TCCATTGGTTGGTACACCAGTTTGTATTAACGGGCTTA |
| | | | TGTTGCTCGAAATCAAAGACACAGAAAAGTACTGTGC |
| | | | CCTTGCACCTAATATGATGGTAACAAACAATACCTTCA |
| | | | CACTCAAAGGCGGTGCACCAACAAAGGTTACTTTTGG |
| | | | TGATGACACTGTGATAGAAGTGCAAGGTTACAAGAGT |
| | | | GTGAATATCACTTTTGAACTTGATGAAAGGATTGATA |
| | | | AAGTACTTAATGAGAAGTGCTCTGCCTATACAGTTGA |
| | | | ACTCGGTACAGAAGTAAATGAGTTCGCCTGTGTTGTG |
| | | | GCAGATGCTGTCATAAAAACTTTGCAACCAGTATCTG |
| | | | AATTACTTACACCACTGGGCATTGATTTAGATGAGTGG |
| | | | AGTATGGCTACATACTACTTATTTGATGAGTCTGGTGA |
| | | | GTTTAAATTGGCTTCACATATGTATTGTTCTTTCTACC |
| | | | CTCCAGATGAGGATGAAGAAGAAGGTGATTGTGAAGA |
| | | | AGAAGAGTTTGAGCCATCAACTCAATATGAGTATGGT |
| | | | ACTGAAGATGATTACCAAGGTAAACCTTTGGAATTTG |
| | | | GTGCCACTTCTGCTGCTCTTCAACCTGAAGAAGAGCA |
| | | | AGAAGAAGATTGGTTAGATGATGATAGTCAACAAACT |
| | | | GTTGGTCAACAAGACGGCAGTGAGGACAATCAGACAA |
| | | | CTACTATTCAAACAATTGTTGAGGTTCAACCTCAATTA |
| | | | GAGATGGAACTTACACCAGTTGTTCAGACTATTGAAG |
| | | | TGAATAGTTTTAGTGGTTATTTAAAACTTACTGACAAT |
| | | | GTATACATTAAAAATGCAGACATTGTGGAAGAAGCTA |
| | | | AAAAGGTAAAACCAACAGTGGTTGTTAATGCAGCCAA |
| | | | TGTTTACCTTAAACATGGAGGAGGTGTTGCAGGAGCC |
| | | | TTAAATAAGGCTACTAACAATGCCATGCAAGTTGAAT |
| | | | CTGATGATTACATAGCTACTAATGGACCACTTAAAGT |
| | | | GGGTGGTAGTTGTGTTTTAAGCGGACACAATCTTGCTA |
| | | | AACACTGTCTTCATGTTGTCGGCCCAAATGTTAACAAA |
| | | | GGTGAAGACATTCAACTTCTTAAGAGTGCTTATGAAA |
| | | | ATTTTAATCAGCACGAAGTTCTACTTGCACCATTATTA |
| | | | TCAGCTGGTATTTTTGGTGCTGACCCTATACATTCTTT |
| | | | AAGAGTTTGTGTAGATACTGTTCGCACAAATGTCTACT |
| | | | TAGCTGTCTTTGATAAAAATCTCTATGACAAACTTGTT |
| | | | TCAAGCTTTTTGGAAATGAAGAGTGAAAAGCAAGTTG |
| | | | AACAAAAGATCGCTGAGATTCCTAAAGAGGAAGTTAA |
| | | | GCCATTTATAACTGAAAGTAAACCTTCAGTTGAACAG |
| | | | AGAAAACAAGATGATAAGAAAATCAAAGCTTGTGTTG |
| | | | AAGAAGTTACAACAACTCTGGAAGAAACTAAGTTCCT |
| | | | CACAGAAAACTTGTTACTTTATATTGACATTAATGGCA |
| | | | ATCTTCATCCAGATTCTGCCACTCTTGTTAGTGACATT |
| | | | GACATCACTTTCTTAAAGAAAGATGCTCCATATATAGT |
| | | | GGGTGATGTTGTTCAAGAGGGTGTTTTAACTGCTGTGG |
| | | | TTATACCTACTAAAAAGGCTGGTGGCACTACTGAAAT |
| | | | GCTAGCGAAAGCTTTGAGAAAAGTGCCAACAGACAAT |
| | | | TATATAACCACTTACCCGGGTCAGGGTTTAAATGGTTA |
| | | | CACTGTAGAGGAGGCAAAGACAGTGCTTAAAAAGTGT |
| | | | AAAAGTGCCTTTTACATTCTACCATCTATTATCTCTAA |
| | | | TGAGAAGCAAGAAATTCTTGGAACTGTTTCTTGGAATT |
| | | | TGCGAGAAATGCTTGCACATGCAGAAGAAACACGCAA |
| | | | ATTAATGCCTGTCTGTGTGGAAACTAAAGCCATAGTTT |
| | | | CAACTATACAGCGTAAATATAAGGGTATTAAAATACA |
| | | | AGAGGGTGTGGTTGATTATGGTGCTAGATTTTACTTTT |
| | | | ACACCAGTAAAACAACTGTAGCGTCACTTATCAACAC |
| | | | ACTTAACGATCTAAATGAAACTCTTGTTACAATGCCAC |
| | | | TTGGCTATGTAACACATGGCTTAAATTTGGAAGAAGC |
| | | | TGCTCGGTATATGAGATCTCTCAAAGTGCCAGCTACA |
| | | | GTTTCTGTTTCTTCACCTGATGCTGTTACAGCGTATAA |
| | | | TGGTTATCTTACTTCTTCTTCTAAAACACCTGAAGAAC |
| | | | ATTTTATTGAAACCATCTCACTTGCTGGTTCCTATAAA |
| | | | GATTGGTCCTATTCTGGACAATCTACACAACTAGGTAT |
| | | | AGAATTTCTTAAGAGAGGTGATAAAAGTGTATATTAC |
| | | | ACTAGTAATCCTACCACATTCCACCTAGATGGTGAAGT |
| | | | TATCACCTTTGACAATCTTAAGACACTTCTTTCTTTGA |
| | | | GAGAAGTGAGGACTATTAAGGTGTTTACAACAGTAGA |
| | | | CAACATTAACCTCCACACGCAAGTTGTGGACATGTCA |
| | | | ATGACATATGGACAACAGTTTGGTCCAACTTATTTGGA |
| | | | TGGAGCTGATGTTACTAAAATAAAACCTCATAATTCA |
| | | | CATGAAGGTAAAACATTTTATGTTTTACCTAATGATGA |
| | | | CACTCTACGTGTTGAGGCTTTTGAGTACTACCACACAA |
| | | | CTGATCCTAGTTTTCTGGGTAGGTACATGTCAGCATTA |
| | | | AATCACACTAAAAAGTGGAAATACCCACAAGTTAATG |
| | | | GTTTAACTTCTATTAAATGGGCAGATAACAACTGTTAT |
| | | | CTTGCCACTGCATTGTTAACACTCCAACAAATAGAGTT |
| | | | GAAGTTTAATCCACCTGCTCTACAAGATGCTTATTACA |
| | | | GAGCAAGGGCTGGTGAAGCTGCTAACTTTTGTGCACT |
| | | | TATCTTAGCCTACTGTAATAAGACAGTAGGTGAGTTA |
| | | | GGTGATGTTAGAGAAACAATGAGTTACTTGTTTCAAC |
| | | | ATGCCAATTTAGATTCTTGCAAAAGAGTCTTGAACGTG |
| | | | GTGTGTAAAACTTGTGGACAACAGCAGACAACCCTTA |
| | | | AGGGTGTAGAAGCTGTTATGTACATGGGCACACTTTCT |
| | | | TATGAACAATTTAAGAAAGGTGTTCAGATACCTTGTA |
| | | | CGTGTGGTAAACAAGCTACAAAATATCTAGTACAACA |
| | | | GGAGTCACCTTTTGTTATGATGTCAGCACCACCTGCTC |
| | | | AGTATGAACTTAAGCATGGTACATTTACTTGTGCTAGT |
| | | | GAGTACACTGGTAATTACCAGTGTGGTCACTATAAAC |
| | | | ATATAACTTCTAAAGAAACTTTGTATTGCATAGACGGT |
| | | | GCTTTACTTACAAAGTCCTCAGAATACAAAGGTCCTAT |
| | | | TACGGATGTTTTCTACAAAGAAAACAGTTACACAACA |
| | | | ACCATAAAACCAGTTACTTATAAATTGGATGGTGTTGT |
| | | | TTGTACAGAAATTGACCCTAAGTTGGACAATTATTATA |
| | | | AGAAAGACAATTCTTATTTCACAGAGCAACCAATTGA |
| | | | TCTTGTACCAAACCAACCATATCCAAACGCAAGCTTC |
| | | | GATAATTTTAAGTTTGTATGTGATAATATCAAATTTGC |
| | | | TGATGATTTAAACCAGTTAACTGGTTATAAGAAACCT |
| | | | GCTTCAAGAGAGCTTAAAGTTACATTTTTCCCTGACTT |
| | | | AAATGGTGATGTGGTGGCTATTGATTATAAACACTAC |
| | | | ACACCCTCTTTTAAGAAAGGAGCTAAATTGTTACATA |
| | | | AACCTATTGTTTGGCATGTTAACAATGCAACTAATAAA |
| | | | GCCACGTATAAACCAAATACCTGGTGTATACGTTGTCT |
| | | | TTGGAGCACAAAACCAGTTGAAACATCAAATTCGTTT |
| | | | GATGTACTGAAGTCAGAGGACGCGCAGGGAATGGATA |
| | | | ATCTTGCCTGCGAAGATCTAAAACCAGTCTCTGAAGA |
| | | | AGTAGTGGAAAATCCTACCATACAGAAAGACGTTCTT |
| | | | GAGTGTAATGTGAAAACTACCGAAGTTGTAGGAGACA |
| | | | TTATACTTAAACCAGCAAATAATAGTTTAAAAATTAC |
| | | | AGAAGAGGTTGGCCACACAGATCTAATGGCTGCTTAT |
| | | | GTAGACAATTCTAGTCTTACTATTAAGAAACCTAATGA |
| | | | ATTATCTAGAGTATTAGGTTTGAAAACCCTTGCTACTC |
| | | | ATGGTTTAGCTGCTGTTAATAGTGTCCCTTGGGATACT |
| | | | ATAGCTAATTATGCTAAGCCTTTTCTTAACAAAGTTGT |
| | | | TAGTACAACTACTAACATAGTTACACGGTGTTTAAACC |
| | | | GTGTTTGTACTAATTATATGCCTTATTTCTTTACTTTA |
| | | | TTGCTACAATTGTGTACTTTTACTAGAAGTACAAATTC |
| | | | TAGAATTAAAGCATCTATGCCGACTACTATAGCAAAGA |
| | | | ATACTGTTAAGAGTGTCGGTAAATTTTGTCTAGAGGCT |
| | | | TCATTTAATTATTTGAAGTCACCTAATTTTTCTAAACT |
| | | | GATAAATATTATAATTTGGTTTTTACTATTAAGTGTTT |
| | | | GCCTAGGTTCTTTAATCTACTCAACCGCTGCTTTAGGT |
| | | | GTTTTAATGTCTAATTTAGGCATGCCTTCTTACTGTAC |
| | | | TGGTTACAGAGAAGGCTATTTGAACTCTACTAATGTCA |
| | | | CTATTGCAACCTACTGTACTGGTTCTATACCTTGTAGT |
| | | | GTTTGTCTTAGTGGTTTAGATTCTTTAGACACCTATCC |
| | | | TTCTTTAGAAACTATACAAATTACCATTTCATCTTTTA |
| | | | AATGGGATTTAACTGCTTTTGGCTTAGTTGCAGAGTGG |
| | | | TTTTTGGCATATATTCTTTTCACTAGGTTTTTCTATGT |
| | | | ACTTGGATTGGCTGCAATCATGCAATTGTTTTTCAGCT |
| | | | ATTTTGCAGTACATTTTATTAGTAATTCTTGGCTTAT |
| | | | GTGGTTAATAATTAATCTTGTACAAATGGCCCCGATTT |
| | | | CAGCTATGGTTAGAATGTACATCTTCTTTGCATCATT |
| | | | TTATTATGTATGGAAAAGTTATGTGCATGTTGTAGACG |
| | | | GTTGTAATTCATCAACTTGTATGATGTGTTACAAACGT |
| | | | AATAGAGCAACAAGAGTCGAATGTACAACTATTGTTAA |
| | | | TGGTGTTAGAAGGTCCTTTTATGTCTATGCTAATGGAG |
| | | | GTAAAGGCTTTTGCAAACTACACAATTGGAATTGTGTT |
| | | | AATTGTGATACATTCTGTGCTGGTAGTACATTTATTAG |
| | | | TGATGAAGTTGCGAGAGACTTGTCACTACAGTTTAAA |
| | | | AGACCAATAAATCCTACTGACCAGTCTTCTTACATCGT |
| | | | TGATAGTGTTACAGTGAAGAATGGTTCCATCCATCTTT |
| | | | ACTTTGATAAAGCTGGTCAAAAGACTTATGAAAGACATTC |
| | | | TCTCTCTCATTTTGTTAACTTAGACAACCTGAGAGCTAA |
| | | | TAACACTAAAGGTTCATTGCCTATTAATGTTATAGTTT |
| | | | TTGATGGTAAATCAAAATGTGAAGAATCATCTGCAAA |
| | | | ATCAGCGTCTGTTTACTACAGTCAGCTTATGTGTCAAC |
| | | | CTATACTGTTACTAGATCAGGCATTAGTGTCTGATGTT |
| | | | GGTGATAGTGCGGAAGTTGCAGTTAAAATGTTTGATG |
| | | | CTTACGTTAATACGTTTTCATCAACTTTTAACGTACCA |
| | | | ATGGAAAAACTCAAAACACTAGTTGCAACTGCAGAAG |
| | | | CTGAACTTGCAAAGAATGTGTCCTTAGACAATGTCTTA |
| | | | TCTACTTTTATTTCAGCAGCTCGGCAAGGGTTTGTTGA |
| | | | TTCAGATGTAGAAACTAAAGATGTTGTTGAATGTCTTA |
| | | | AATTGTCACATCAATCTGACATAGAAGTTACTGGCGA |
| | | | TAGTTGTAATAACTATATGCTCACCTATAACAAAGTTG |
| | | | AAAACATGACACCCCGTGACCTTGGTGCTTGTATTGAC |
| | | | TGTAGTGCGCGTCATATTAATGCGCAGGTAGCAAAAA |
| | | | GTCACAACATTGCTTTGATATGGAACGTTAAAGATTTC |
| | | | ATGTCATTGTCTGAACAACTACGAAAACAAATACGTA |
| | | | GTGCTGCTAAAAAGAATAACTTACCTTTTAAGTTGACA |
| | | | TGTGCAACTACTAGACAAGTTGTTAATGTTGTAACAAC |
| | | | AAAGATAGCACTTAAGGGTGGTAAAATTGTTAATAAT |
| | | | TGGTTGAAGCAGTTAATTAAAGTTACACTTGTGTTCCT |
| | | | TTTTGTTGCTGCTATTTTCTATTTAATAACACCTGTTCA |
| | | | TGTCATGTCTAAACATACTGACTTTTCAAGTGAAATCA |
| | | | TAGGATACAAGGCTATTGATGGTGGTGTCACTCGTGA |
| | | | CATAGCATCTACAGATACTTGTTTTGCTAACAAACATG |
| | | | CTGATTTTGACACATGGTTTAGCCAGCGTGGTGGTAGT |
| | | | TATACTAATGACAAAGCTTGCCCATTGATTGCTGCAGT |
| | | | CATAACAAGAGAAGTGGGTTTTGTCGTGCCTGGTTTGC |
| | | | CTGGCACGATATTACGCACAACTAATGGTGACTTTTTG |
| | | | CATTTCTTACCTAGAGTTTTTAGTGCAGTTGGTAACAT |
| | | | CTGTTACACACCATCAAAACTTATAGAGTACACTGACT |
| | | | TTGCAACATCAGCTTGTGTTTTGGCTGCTGAATGTACA |
| | | | ATTTTTAAAGATGCTTCTGGTAAGCCAGTACCATATTG |
| | | | TTATGATACCAATGTACTAGAAGGTTCTGTTGCTTATG |
| | | | AAAGTTTACGCCCTGACACACGTTATGTGCTCATGGAT |
| | | | GGCTCTATTATTCAATTTCCTAACACCTACCTTGAAGG |
| | | | TTCTGTTAGAGTGGTAACAACTTTTGATTCTGAGTACT |
| | | | GTAGGCACGGCACTTGTGAAAGATCAGAAGCTGGTGT |
| | | | TTGTGTATCTACTAGTGGTAGATGGGTACTTAACAATG |
| | | | ATTATTACAGATCTTTACCAGGAGTTTTCTGTGGTGTA |
| | | | GATGCTGTAAATTTACTTACTAATATGTTTACACCACT |
| | | | AATTCAACCTATTGGTGCTTTGGACATATCAGCATCTA |
| | | | TAGTAGCTGGTGGTATTGTAGCTATCGTAGTAACATGC |
| | | | CTTGCCTACTATTTTATGAGGTTTAGAAGAGCTTTTGG |
| | | | TGAATACAGTCATGTAGTTGCCTTTAATACTTTACTAT |
| | | | TCCTTATGTCATTCACTGTACTCTGTTTAACACCAGTTT |
| | | | ACTCATTCTTACCTGGTGTTTATTCTGTTATTTACTTGT |
| | | | ACTTGACATTTTATCTTACTAATGATGTTTCTTTTTTAG |
| | | | CACATATTCAGTGGATGGTTATGTTCACACCTTTAGTA |
| | | | CCTTTCTGGATAACAATTGCTTATATCATTTGTATTTCC |
| | | | ACAAAGCATTTCTATTGGTTCTTTAGTAATTACCTAAA |
| | | | GAGACGTGTAGTCTTTAATGGTGTTTCCTTTAGTACTT |
| | | | TTGAAGAAGCTGCGCTGTGCACCTTTTTGTTAAATAAA |
| | | | GAAATGTATCTAAAGTTGCGTAGTGATGTGCTATTACC |
| | | | TCTTACGCAATATAATAGATACTTAGCTCTTTATAATA |
| | | | AGTACAAGTATTTTAGTGGAGCAATGGATACAACTAG |
| | | | CTACAGAGAAGCTGCTTGTTGTCATCTCGCAAAGGCTC |
| | | | TCAATGACTTCAGTAACTCAGGTTCTGATGTTCTTTAC |
| | | | CAACCACCACAAACCTCTATCACCTCAGCTGTTTTGCA |
| | | | GAGTGGTTTTAGAAAAATGGCATTCCCATCTGGTAAA |
| | | | GTTGAGGGTTGTATGGTACAAGTAACTTGTGGTACAA |
| | | | CTACACTTAACGGTCTTTGGCTTGATGACGTAGTTTAC |
| | | | TGTCCAAGACATGTGATCTGCACCTCTGAAGACATGCT |
| | | | TAACCCTAATTATGAAGATTTACTCATTCGTAAGTCTA |
| | | | ATCATAATTTCTTGGTACAGGCTGGTAATGTTCAACTC |
| | | | AGGGTTATTGGACATTCTATGCAAAATTGTGTACTTAA |
| | | | GCTTAAGGTTGATACAGCCAATCCTAAGACACCTAAG |
| | | | TATAAGTTTGTTCGCATTCAACCAGGACAGACTTTTTC |
| | | | AGTGTTAGCTTGTTACAATGGTTCACCATCTGGTGTTT |
| | | | ACCAATGTGCTATGAGGCCCAATTTCACTATTAAGGGT |
| | | | TCATTCCTTAATGGTTCATGTGGTAGTGTTGGTTTTAA |
| | | | CATAGATTATGACTGTGTCTCTTTTTGTTACATGCACC |
| | | | ATATGGAATTACCAACTGGAGTTCATGCTGGCACAGA |
| | | | CTTAGAAGGTAACTTTTATGGACCTTTTGTTGACAGGC |
| | | | AAACAGCACAAGCAGCTGGTACGGACACAACTATTAC |
| | | | AGTTAATGTTTTAGCTTGGTTGTACGCTGCTGTTATAA |
| | | | ATGGAGACAGGTGGTTTCTCAATCGATTTACCACAACT |
| | | | CTTAATGACTTTAACCTTGTGGCTATGAAGTACAATTA |
| | | | TGAACCTCTAACACAAGACCATGTTGACATACTAGGA |
| | | | CCTCTTTCTGCTCAAACTGGAATTGCCGTTTTAGATAT |
| | | | GTGTGCTTCATTAAAAGAATTACTGCAAAATGGTATG |
| | | | AATGGACGTACCATATTGGGTAGTGCTTTATTAGAAG |
| | | | ATGAATTTACACCTTTTGATGTTGTTAGACAATGCTCA |
| | | | GGTGTTACTTTCCAAAGTGCAGTGAAAAGAACAATCA |
| | | | AGGGTACACACCACTGGTTGTTACTCACAATTTTGACT |
| | | | TCACTTTTAGTTTTAGTCCAGAGTACTCAATGGTCTTT |
| | | | GTTCTTTTTTTTGTATGAAAATGCCTTTTTACCTTTTGC |
| | | | TATGGGTATTATTGCTATGTCTGCTTTTGCAATGATGT |
| | | | TTGTCAAACATAAGCATGCATTTCTCTGTTTGTTTTTGT |
| | | | TACCTTCTCTTGCCACTGTAGCTTATTTTAATATGGTCT |
| | | | ATATGCCTGCTAGTTGGGTGATGCGTATTATGACATGG |
| | | | TTGGATATGGTTGATACTAGTTTGTCTGGTTTTAAGCT |
| | | | AAAAGACTGTGTTATGTATGCATCAGCTGTAGTGTTAC |
| | | | TAATCCTTATGACAGCAAGAACTGTGTATGATGATGG |
| | | | TGCTAGGAGAGTGTGGACACTTATGAATGTCTTGACA |
| | | | CTCGTTTATAAAGTTTATTATGGTAATGCTTTAGATCA |
| | | | AGCCATTTCCATGTGGGCTCTTATAATCTCTGTTACTT |
| | | | CTAACTACTCAGGTGTAGTTACAACTGTCATGTTTTTG |
| | | | GCCAGAGGTATTGTTTTTATGTGTGTTGAGTATTGCCC |
| | | | TATTTTCTTCATAACTGGTAATACACTTCAGTGTATAA |
| | | | TGCTAGTTTATTGTTTCTTAGGCTATTTTTGTACTTGTT |
| | | | ACTTTGGCCTCTTTTGTTTACTCAACCGCTACTTTAGAC |
| | | | TGACTCTTGGTGTTTATGATTACTTAGTTTCTACACAG |
| | | | GAGTTTAGATATATGAATTCACAGGGACTACTCCCAC |
| | | | CCAAGAATAGCATAGATGCCTTCAAACTCAACATTAA |
| | | | ATTGTTGGGTGTTGGTGGCAAACCTTGTATCAAAGTAG |
| | | | CCACTGTACAGTCTAAAATGTCAGATGTAAAGTGCAC |
| | | | ATCAGTAGTCTTACTCTCAGTTTTGCAACAACTCAGAG |
| | | | TAGAATCATCATCTAAATTGTGGGCTCAATGTGTCCAG |
| | | | TTACACAATGACATTCTCTTAGCTAAAGATACTACTGA |
| | | | AGCCTTTGAAAAAATGGTTTCACTACTTTCTGTTTTGC |
| | | | TTTCCATGCAGGGTGCTGTAGACATAAACAAGCTTTGT |
| | | | GAAGAAATGCTGGACAACAGGGCAACCTTACAAGCTA |
| | | | TAGCCTCAGAGTTTAGTTCCCTTCCATCATATGCAGCT |
| | | | TTTGCTACTGCTCAAGAAGCTTATGAGCAGGCTGTTGC |
| | | | TAATGGTGATTCTGAAGTTGTTCTTAAAAAGTTGAAGA |
| | | | AGTCTTTGAATGTGGCTAAATCTGAATTTGACCGTGAT |
| | | | GCAGCCATGCAACGTAAGTTGGAAAAGATGGCTGATC |
| | | | AAGCTATGACCCAAATGTATAAACAGGCTAGATCTGA |
| | | | GGACAAGAGGGCAAAAGTTACTAGTGCTATGCAGACA |
| | | | ATGCTTTTCACTATGCTTAGAAAGTTGGATAATGATGC |
| | | | ACTCAACAACATTATCAACAATGCAAGAGATGGTTGT |
| | | | GTTCCCTTGAACATAATACCTCTTACAACAGCAGCCAA |
| | | | ACTAATGGTTGTCATACCAGACTATAACACATATAAA |
| | | | AATACGTGTGATGGTACAACATTTACTTATGCATCAGC |
| | | | ATTGTGGGAAATCCAACAGGTTGTAGATGCAGATAGT |
| | | | AAAATTGTTCAACTTAGTGAAATTAGTATGGACAATTC |
| | | | ACCTAATTTAGCATGGCCTCTTATTGTAACAGCTTTAA |
| | | | GGGCCAATTCTGCTGTCAAATTACAGAATAATGAGCT |
| | | | TAGTCCTGTTGCACTACGACAGATGTCTTGTGCTGCCG |
| | | | GTACTACACAAACTGCTTGCACTGATGACAATGCGTT |
| | | | AGCTTACTACAACACAACAAAGGGAGGTAGGTTTGTA |
| | | | CTTGCACTGTTATCCGATTTACAGGATTTGAAATGGGC |
| | | | TAGATTCCCTAAGAGTGATGGAACTGGTACTATCTATA |
| | | | CAGAACTGGAACCACCTTGTAGGTTTGTTACAGACAC |
| | | | ACCTAAAGGTCCTAAAGTGAAGTATTTATACTTTATTA |
| | | | AAGGATTAAACAACCTAAATAGAGGTATGGTACTTGG |
| | | | TAGTTTAGCTGCCACAGTACGTCTACAAGCTGGTAATG |
| | | | CAACAGAAGTGCCTGCCAATTCAACTGTATTATCTTTC |
| | | | TGTGCTTTTGCTGTAGATGCTGCTAAAGCTTACAAAGA |
| | | | TTATCTAGCTAGTGGGGGACAACCAATCACTAATTGT |
| | | | GTTAAGATGTTGTGTACACACACTGGTACTGGTCAGG |
| | | | CAATAACAGTTACACCGGAAGCCAATATGGATCAAGA |
| | | | ATCCTTTGGTGGTGCATCGTGTTGTCTGTACTGCCGTT |
| | | | GCCACATAGATCATCCAAATCCTAAAGGATTTTGTGA |
| | | | CTTAAAAGGTAAGTATGTACAAATACCTACAACTTGT |
| | | | GCTAATGACCCTGTGGGTTTTACACTTAAAAACACAGT |
| | | | CTGTACCGTCTGCGGTATGTGGAAAGGTTATGGCTGTA |
| | | | GTTGTGATCAACTCCGCGAACCCATGCTTCAGTCAGCT |
| | | | GATGCACAATCGTTTTTAAACGGGTTTGCGGTGTA |
| orf1ab | 266 | 21555 | ATGGAGAGCCTTGTCCCTGGTTTCAACGAGAAAACAC |
| (SEQ ID | | | ACGTCCAACTCAGTTTGCCTGTTTTACAGGTTCGCGAC |
| NO: 3) | | | GTGCTCGTACGTGGCTTTGGAGACTCCGTGGAGGAGG |
| | | | TCTTATCAGAGGCACGTCAACATCTTAAAGATGGCAC |
| | | | TTGTGGCTTAGTAGAAGTTGAAAAAGGCGTTTTGCCTC |
| | | | AACTTGAACAGCCCTATGTGTTCATCAAACGTTCGGAT |
| | | | GCTCGAACTGCACCTCATGGTCATGTTATGGTTGAGCT |
| | | | GGTAGCAGAACTCGAAGGCATTCAGTACGGTCGTAGT |
| | | | GGTGAGACACTTGGTGTCCTTGTCCCTCATGTGGGCGA |
| | | | AATACCAGTGGCTTACCGCAAGGTTCTTCTTCGTAAGA |
| | | | ACGGTAATAAAGGAGCTGGTGGCCATAGTTACGGCGC |
| | | | CGATCTAAAGTCATTTGACTTAGGCGACGAGCTTGGC |
| | | | ACTGATCCTTATGAAGATTTTCAAGAAAACTGGAACA |
| | | | CTAAACATAGCAGTGGTGTTACCCGTGAACTCATGCG |
| | | | TGAGCTTAACGGAGGGGCATACACTCGCTATGTCGAT |
| | | | AACAACTTCTGTGGCCCTGATGGCTACCCTCTTGAGTG |
| | | | CATTAAAGACCTTCTAGCACGTGCTGGTAAAGCTTCAT |
| | | | GCACTTTGTCCGAACAACTGGACTTTATTGACACTAAG |
| | | | AGGGGTGTATACTGCTGCCGTGAACATGAGCATGAAA |
| | | | TTGCTTGGTACACGGAACGTTCTGAAAAGAGCTATGA |
| | | | ATTGCAGACACCTTTTGAAATTAAATTGGCAAAGAAA |
| | | | TTTGACACCTTCAATGGGGAATGTCCAAATTTTGTATT |
| | | | TCCCTTAAATTCCATAATCAAGACTATTCAACCAAGGG |
| | | | TTGAAAAGAAAAAGCTTGATGGCTTTATGGGTAGAAT |
| | | | TCGATCTGTCTATCCAGTTGCGTCACCAAATGAATGCA |
| | | | ACCAAATGTGCCTTTCAACTCTCATGAAGTGTGATCAT |
| | | | TGTGGTGAAACTTCATGGCAGACGGGCGATTTTGTTA |
| | | | AAGCCACTTGCGAATTTTGTGGCACTGAGAATTTGACT |
| | | | AAAGAAGGTGCCACTACTTGTGGTTACTTACCCCAAA |
| | | | ATGCTGTTGTTAAAATTTATTGTCCAGCATGTCACAAT |
| | | | TCAGAAGTAGGACCTGAGCATAGTCTTGCCGAATACC |
| | | | ATAATGAATCTGGCTTGAAAACCATTCTTCGTAAGGGT |
| | | | GGTCGCACTATTGCCTTTGGAGGCTGTGTGTTCTCTTA |
| | | | TGTTGGTTGCCATAACAAGTGTGCCTATTGGGTTCCAC |
| | | | GTGCTAGCGCTAACATAGGTTGTAACCATACAGGTGT |
| | | | TGTTGGAGAAGGTTCCGAAGGTCTTAATGACAACCTT |
| | | | CTTGAAATACTCCAAAAAGAGAAAGTCAACATCAATA |
| | | | TTGTTGGTGACTTTAAACTTAATGAAGAGATCGCCATT |
| | | | ATTTTGGCATCTTTTTCTGCTTCCACAAGTGCTTTTGTG |
| | | | GAAACTGTGAAAGGTTTGGATTATAAAGCATTCAAAC |
| | | | AAATTGTTGAATCCTGTGGTAATTTTAAAGTTACAAAA |
| | | | GGAAAAGCTAAAAAAGGTGCCTGGAATATTGGTGAAC |
| | | | AGAAATCAATACTGAGTCCTCTTTATGCATTTGCATCA |
| | | | GAGGCTGCTCGTGTTGTACGATCAATTTTCTCCCGCAC |
| | | | TCTTGAAACTGCTCAAAATTCTGTGCGTGTTTTACAGA |
| | | | AGGCCGCTATAACAATACTAGATGGAATTTCACAGTA |
| | | | TTCACTGAGACTCATTGATGCTATGATGTTCACATCTG |
| | | | ATTTGGCTACTAACAATCTAGTTGTAATGGCCTACATT |
| | | | ACAGGTGGTGTTGTTCAGTTGACTTCGCAGTGGCTAAC |
| | | | TAACATCTTTGGCACTGTTTATGAAAAACTCAAACCCG |
| | | | TCCTTGATTGGCTTGAAGAGAAGTTTAAGGAAGGTGT |
| | | | AGAGTTTCTTAGAGACGGTTGGGAAATTGTTAAATTTA |
| | | | TCTCAACCTGTGCTTGTGAAATTGTCGGTGGACAAATT |
| | | | GTCACCTGTGCAAAGGAAATTAAGGAGAGTGTTCAGA |
| | | | CATTCTTTAAGCTTGTAAATAAATTTTTGGCTTTGTGT |
| | | | GCTGACTCTATCATTATTGGTGGAGCTAAACTTAAAGC |
| | | | CTTGAATTTAGGTGAAACATTTGTCACGCACTCAAAG |
| | | | GGATTGTACAGAAAGTGTGTTAAATCCAGAGAAGAAA |
| | | | CTGGCCTACTCATGCCTCTAAAAGCCCCAAAAGAAAT |
| | | | TATCTTCTTAGAGGGAGAAACACTTCCCACAGAAGTG |
| | | | TTAACAGAGGAAGTTGTCTTGAAAACTGGTGATTTAC |
| | | | AACCATTAGAACAACCTACTAGTGAAGCTGTTGAAGC |
| | | | TCCATTGGTTGGTACACCAGTTTGTATTAACGGGCTTA |
| | | | TGTTGCTCGAAATCAAAGACACAGAAAAGTACTGTGC |
| | | | CCTTGCACCTAATATGATGGTAACAAACAATACCTTCA |
| | | | CACTCAAAGGCGGTGCACCAACAAAGGTTACTTTTGG |
| | | | TGATGACACTGTGATAGAAGTGCAAGGTTACAAGAGT |
| | | | GTGAATATCACTTTTGAACTTGATGAAAGGATTGATA |
| | | | AAGTACTTAATGAGAAGTGCTCTGCCTATACAGTTGA |
| | | | ACTCGGTACAGAAGTAAATGAGTTCGCCTGTGTTGTG |
| | | | GCAGATGCTGTCATAAAAACTTTGCAACCAGTATCTG |
| | | | AATTACTTACACCACTGGGCATTGATTTAGATGAGTGG |
| | | | AGTATGGCTACATACTACTTATTTGATGAGTCTGGTGA |
| | | | GTTTAAATTGGCTTCACATATGTATTGTTCTTTCTACCC |
| | | | TCCAGATGAGGATGAAGAAGAAGGTGATTGTGAAGA |
| | | | AGAAGAGTTTGAGCCATCAACTCAATATGAGTATGGT |
| | | | ACTGAAGATGATTACCAAGGTAAACCTTTGGAATTTG |
| | | | GTGCCACTTCTGCTGCTCTTCAACCTGAAGAAGAGCA |
| | | | AGAAGAAGATTGGTTAGATGATGATAGTCAACAAACT |
| | | | GTTGGTCAACAAGACGGCAGTGAGGACAATCAGACAA |
| | | | CTACTATTCAAACAATTGTTGAGGTTCAACCTCAATTA |
| | | | GAGATGGAACTTACACCAGTTGTTCAGACTATTGAAG |
| | | | TGAATAGTTTTAGTGGTTATTTAAAACTTACTGACAAT |
| | | | GTATACATTAAAAATGCAGACATTGTGGAAGAAGCTA |
| | | | AAAAGGTAAAACCAACAGTGGTTGTTAATGCAGCCAA |
| | | | TGTTTACCTTAAACATGGAGGAGGTGTTGCAGGAGCC |
| | | | TTAAATAAGGCTACTAACAATGCCATGCAAGTTGAAT |
| | | | CTGATGATTACATAGCTACTAATGGACCACTTAAAGT |
| | | | GGGTGGTAGTTGTGTTTTAAGCGGACACAATCTTGCTA |
| | | | AACACTGTCTTCATGTTGTCGGCCCAAATGTTAACAAA |
| | | | GGTGAAGACATTCAACTTCTTAAGAGTGCTTATGAAA |
| | | | ATTTTAATCAGCACGAAGTTCTACTTGCACCATTATTA |
| | | | TCAGCTGGTATTTTTGGTGCTGACCCTATACATTCTTT |
| | | | AAGAGTTTGTGTAGATACTGTTCGCACAAATGTCTACT |
| | | | TAGCTGTCTTTGATAAAAATCTCTATGACAAACTTGTT |
| | | | TCAAGCTTTTTGGAAATGAAGAGTGAAAAGCAAGTTG |
| | | | AACAAAAGATCGCTGAGATTCCTAAAGAGGAAGTTAA |
| | | | GCCATTTATAACTGAAAGTAAACCTTCAGTTGAACAG |
| | | | AGAAAACAAGATGATAAGAAAATCAAAGCTTGTGTTG |
| | | | AAGAAGTTACAACAACTCTGGAAGAAACTAAGTTCCT |
| | | | CACAGAAAACTTGTTACTTTATATTGACATTAATGGCA |
| | | | ATCTTCATCCAGATTCTGCCACTCTTGTTAGTGACATT |
| | | | GACATCACTTTCTTAAAGAAAGATGCTCCATATATAGT |
| | | | GGGTGATGTTGTTCAAGAGGGTGTTTTAACTGCTGTGG |
| | | | TTATACCTACTAAAAAGGCTGGTGGCACTACTGAAAT |
| | | | GCTAGCGAAAGCTTTGAGAAAAGTGCCAACAGACAAT |
| | | | TATATAACCACTTACCCGGGTCAGGGTTTAAATGGTTA |
| | | | CACTGTAGAGGAGGCAAAGACAGTGCTTAAAAAGTGT |
| | | | AAAAGTGCCTTTTACATTCTACCATCTATTATCTCTAA |
| | | | TGAGAAGCAAGAAATTCTTGGAACTGTTTCTTGGAATT |
| | | | TGCGAGAAATGCTTGCACATGCAGAAGAAACACGCAA |
| | | | ATTAATGCCTGTCTGTGTGGAAACTAAAGCCATAGTTT |
| | | | CAACTATACAGCGTAAATATAAGGGTATTAAAATACA |
| | | | AGAGGGTGTGGTTGATTATGGTGCTAGATTTTACTTTT |
| | | | ACACCAGTAAAACAACTGTAGCGTCACTTATCAACAC |
| | | | ACTTAACGATCTAAATGAAACTCTTGTTACAATGCCAC |
| | | | TTGGCTATGTAACACATGGCTTAAATTTGGAAGAAGC |
| | | | TGCTCGGTATATGAGATCTCTCAAAGTGCCAGCTACA |
| | | | GTTTCTGTTTCTTCACCTGATGCTGTTACAGCGTATAA |
| | | | TGGTTATCTTACTTCTTCTTCTAAAACACCTGAAGAAC |
| | | | ATTTTATTGAAACCATCTCACTTGCTGGTTCCTATAAA |
| | | | GATTGGTCCTATTCTGGACAATCTACACAACTAGGTAT |
| | | | AGAATTTCTTAAGAGAGGTGATAAAAGTGTATATTAC |
| | | | ACTAGTAATCCTACCACATTCCACCTAGATGGTGAAGT |
| | | | TATCACCTTTGACAATCTTAAGACACTTCTTTCTTTGA |
| | | | GAGAAGTGAGGACTATTAAGGTGTTTACAACAGTAGA |
| | | | CAACATTAACCTCCACACGCAAGTTGTGGACATGTCA |
| | | | ATGACATATGGACAACAGTTTGGTCCAACTTATTTGGA |
| | | | TGGAGCTGATGTTACTAAAATAAAACCTCATAATTCA |
| | | | CATGAAGGTAAAACATTTTATGTTTTACCTAATGATGA |
| | | | CACTCTACGTGTTGAGGCTTTTGAGTACTACCACACAA |
| | | | CTGATCCTAGTTTTCTGGGTAGGTACATGTCAGCATTA |
| | | | AATCACACTAAAAAGTGGAAATACCCACAAGTTAATG |
| | | | GTTTAACTTCTATTAAATGGGCAGATAACAACTGTTAT |
| | | | CTTGCCACTGCATTGTTAACACTCCAACAAATAGAGTT |
| | | | GAAGTTTAATCCACCTGCTCTACAAGATGCTTATTACA |
| | | | GAGCAAGGGCTGGTGAAGCTGCTAACTTTTGTGCACT |
| | | | TATCTTAGCCTACTGTAATAAGACAGTAGGTGAGTTA |
| | | | GGTGATGTTAGAGAAACAATGAGTTACTTGTTTCAAC |
| | | | ATGCCAATTTAGATTCTTGCAAAAGAGTCTTGAACGTG |
| | | | GTGTGTAAAACTTGTGGACAACAGCAGACAACCCTTA |
| | | | AGGGTGTAGAAGCTGTTATGTACATGGGCACACTTTCT |
| | | | TATGAACAATTTAAGAAAGGTGTTCAGATACCTTGTA |
| | | | CGTGTGGTAAACAAGCTACAAAATATCTAGTACAACA |
| | | | GGAGTCACCTTTTGTTATGATGTCAGCACCACCTGCTC |
| | | | AGTATGAACTTAAGCATGGTACATTTACTTGTGCTAGT |
| | | | GAGTACACTGGTAATTACCAGTGTGGTCACTATAAAC |
| | | | ATATAACTTCTAAAGAAACTTTGTATTGCATAGACGGT |
| | | | GCTTTACTTACAAAGTCCTCAGAATACAAAGGTCCTAT |
| | | | TACGGATGTTTTCTACAAAGAAAACAGTTACACAACA |
| | | | ACCATAAAACCAGTTACTTATAAATTGGATGGTGTTGT |
| | | | TTGTACAGAAATTGACCCTAAGTTGGACAATTATTATA |
| | | | AGAAAGACAATTCTTATTTCACAGAGCAACCAATTGA |
| | | | TCTTGTACCAAACCAACCATATCCAAACGCAAGCTTC |
| | | | GATAATTTTAAGTTTGTATGTGATAATATCAAATTTGC |
| | | | TGATGATTTAAACCAGTTAACTGGTTATAAGAAACCT |
| | | | GCTTCAAGAGAGCTTAAAGTTACATTTTTCCCTGACTT |
| | | | AAATGGTGATGTGGTGGCTATTGATTATAAACACTAC |
| | | | ACACCCTCTTTTAAGAAAGGAGCTAAATTGTTACATA |
| | | | AACCTATTGTTTGGCATGTTAACAATGCAACTAATAAA |
| | | | GCCACGTATAAACCAAATACCTGGTGTATACGTTGTCT |
| | | | TTGGAGCACAAAACCAGTTGAAACATCAAATTCGTTT |
| | | | GATGTACTGAAGTCAGAGGACGCGCAGGGAATGGATA |
| | | | ATCTTGCCTGCGAAGATCTAAAACCAGTCTCTGAAGA |
| | | | AGTAGTGGAAAATCCTACCATACAGAAAGACGTTCTT |
| | | | GAGTGTAATGTGAAAACTACCGAAGTTGTAGGAGACA |
| | | | TTATACTTAAACCAGCAAATAATAGTTTAAAAATTAC |
| | | | AGAAGAGGTTGGCCACACAGATCTAATGGCTGCTTAT |
| | | | GTAGACAATTCTAGTCTTACTATTAAGAAACCTAATGA |
| | | | ATTATCTAGAGTATTAGGTTTGAAAACCCTTGCTACTC |
| | | | ATGGTTTAGCTGCTGTTAATAGTGTCCCTTGGGATACT |
| | | | ATAGCTAATTATGCTAAGCCTTTTCTTAACAAAGTTGT |
| | | | TAGTACAACTACTAACATAGTTACACGGTGTTTAAACC |
| | | | GTGTTTGTACTAATTATATGCCTTATTTCTTTACTTTAT |
| | | | TGCTACAATTGTGTACTTTTACTAGAAGTACAAATTCT |
| | | | AGAATTAAAGCATCTATGCCGACTACTATAGCAAAGA |
| | | | ATACTGTTAAGAGTGTCGGTAAATTTTGTCTAGAGGCT |
| | | | TCATTTAATTATTTGAAGTCACCTAATTTTTCTAAACT |
| | | | GATAAATATTATAATTTGGTTTTTACTATTAAGTGTTT |
| | | | GCCTAGGTTCTTTAATCTACTCAACCGCTGCTTTAGGT |
| | | | GTTTTAATGTCTAATTTAGGCATGCCTTCTTACTGTACT |
| | | | GGTTACAGAGAAGGCTATTTGAACTCTACTAATGTCA |
| | | | CTATTGCAACCTACTGTACTGGTTCTATACCTTGTAGT |
| | | | GTTTGTCTTAGTGGTTTAGATTCTTTAGACACCTATCCT |
| | | | TCTTTAGAAACTATACAAATTACCATTTCATCTTTTAA |
| | | | ATGGGATTTAACTGCTTTTGGCTTAGTTGCAGAGTGGT |
| | | | TTTTGGCATATATTCTTTTCACTAGGTTTTTCTATGTAC |
| | | | TTGGATTGGCTGCAATCATGCAATTGTTTTTCAGCTAT |
| | | | TTTGCAGTACATTTTATTAGTAATTCTTGGCTTATGTG |
| | | | GTTAATAATTAATCTTGTACAAATGGCCCCGATTTCAG |
| | | | CTATGGTTAGAATGTACATCTTCTTTGCATCATTTTATT |
| | | | ATGTATGGAAAAGTTATGTGCATGTTGTAGACGGTTGT |
| | | | AATTCATCAACTTGTATGATGTGTTACAAACGTAATAG |
| | | | AGCAACAAGAGTCGAATGTACAACTATTGTTAATGGT |
| | | | GTTAGAAGGTCCTTTTATGTCTATGCTAATGGAGGTAA |
| | | | AGGCTTTTGCAAACTACACAATTGGAATTGTGTTAATT |
| | | | GTGATACATTCTGTGCTGGTAGTACATTTATTAGTGAT |
| | | | GAAGTTGCGAGAGACTTGTCACTACAGTTTAAAAGAC |
| | | | CAATAAATCCTACTGACCAGTCTTCTTACATCGTTGAT |
| | | | AGTGTTACAGTGAAGAATGGTTCCATCCATCTTTACTT |
| | | | TGATAAAGCTGGTCAAAAGACTTATGAAAGACATTCT |
| | | | CTCTCTCATTTTGTTAACTTAGACAACCTGAGAGCTAA |
| | | | TAACACTAAAGGTTCATTGCCTATTAATGTTATAGTTT |
| | | | TTGATGGTAAATCAAAATGTGAAGAATCATCTGCAAA |
| | | | ATCAGCGTCTGTTTACTACAGTCAGCTTATGTGTCAAC |
| | | | CTATACTGTTACTAGATCAGGCATTAGTGTCTGATGTT |
| | | | GGTGATAGTGCGGAAGTTGCAGTTAAAATGTTTGATG |
| | | | CTTACGTTAATACGTTTTCATCAACTTTTAACGTACCA |
| | | | ATGGAAAAACTCAAAACACTAGTTGCAACTGCAGAAG |
| | | | CTGAACTTGCAAAGAATGTGTCCTTAGACAATGTCTTA |
| | | | TCTACTTTTATTTCAGCAGCTCGGCAAGGGTTTGTTGA |
| | | | TTCAGATGTAGAAACTAAAGATGTTGTTGAATGTCTTA |
| | | | AATTGTCACATCAATCTGACATAGAAGTTACTGGCGA |
| | | | TAGTTGTAATAACTATATGCTCACCTATAACAAAGTTG |
| | | | AAAACATGACACCCCGTGACCTTGGTGCTTGTATTGAC |
| | | | TGTAGTGCGCGTCATATTAATGCGCAGGTAGCAAAAA |
| | | | GTCACAACATTGCTTTGATATGGAACGTTAAAGATTTC |
| | | | ATGTCATTGTCTGAACAACTACGAAAACAAATACGTA |
| | | | GTGCTGCTAAAAAGAATAACTTACCTTTTAAGTTGACA |
| | | | TGTGCAACTACTAGACAAGTTGTTAATGTTGTAACAAC |
| | | | AAAGATAGCACTTAAGGGTGGTAAAATTGTTAATAAT |
| | | | TGGTTGAAGCAGTTAATTAAAGTTACACTTGTGTTCCT |
| | | | TTTTGTTGCTGCTATTTTCTATTTAATAACACCTGTTCA |
| | | | TGTCATGTCTAAACATACTGACTTTTCAAGTGAAATCA |
| | | | TAGGATACAAGGCTATTGATGGTGGTGTCACTCGTGA |
| | | | CATAGCATCTACAGATACTTGTTTTGCTAACAAACATG |
| | | | CTGATTTTGACACATGGTTTAGCCAGCGTGGTGGTAGT |
| | | | TATACTAATGACAAAGCTTGCCCATTGATTGCTGCAGT |
| | | | CATAACAAGAGAAGTGGGTTTTGTCGTGCCTGGTTTGC |
| | | | CTGGCACGATATTACGCACAACTAATGGTGACTTTTTG |
| | | | CATTTCTTACCTAGAGTTTTTAGTGCAGTTGGTAACAT |
| | | | CTGTTACACACCATCAAAACTTATAGAGTACACTGACT |
| | | | TTGCAACATCAGCTTGTGTTTTGGCTGCTGAATGTACA |
| | | | ATTTTTAAAGATGCTTCTGGTAAGCCAGTACCATATTG |
| | | | TTATGATACCAATGTACTAGAAGGTTCTGTTGCTTATG |
| | | | AAAGTTTACGCCCTGACACACGTTATGTGCTCATGGAT |
| | | | GGCTCTATTATTCAATTTCCTAACACCTACCTTGAAGG |
| | | | TTCTGTTAGAGTGGTAACAACTTTTGATTCTGAGTACT |
| | | | GTAGGCACGGCACTTGTGAAAGATCAGAAGCTGGTGT |
| | | | TTGTGTATCTACTAGTGGTAGATGGGTACTTAACAATG |
| | | | ATTATTACAGATCTTTACCAGGAGTTTTCTGTGGTGTA |
| | | | GATGCTGTAAATTTACTTACTAATATGTTTACACCACT |
| | | | AATTCAACCTATTGGTGCTTTGGACATATCAGCATCTA |
| | | | TAGTAGCTGGTGGTATTGTAGCTATCGTAGTAACATGC |
| | | | CTTGCCTACTATTTTATGAGGTTTAGAAGAGCTTTTGG |
| | | | TGAATACAGTCATGTAGTTGCCTTTAATACTTTACTAT |
| | | | TCCTTATGTCATTCACTGTACTCTGTTTAACACCAGTTT |
| | | | ACTCATTCTTACCTGGTGTTTATTCTGTTATTTACTTGT |
| | | | ACTTGACATTTTATCTTACTAATGATGTTTCTTTTTTAG |
| | | | CACATATTCAGTGGATGGTTATGTTCACACCTTTAGTA |
| | | | CCTTTCTGGATAACAATTGCTTATATCATTTGTATTTCC |
| | | | ACAAAGCATTTCTATTGGTTCTTTAGTAATTACCTAAA |
| | | | GAGACGTGTAGTCTTTAATGGTGTTTCCTTTAGTACTT |
| | | | TTGAAGAAGCTGCGCTGTGCACCTTTTTGTTAAATAAA |
| | | | GAAATGTATCTAAAGTTGCGTAGTGATGTGCTATTACC |
| | | | TCTTACGCAATATAATAGATACTTAGCTCTTTATAATA |
| | | | AGTACAAGTATTTTAGTGGAGCAATGGATACAACTAG |
| | | | CTACAGAGAAGCTGCTTGTTGTCATCTCGCAAAGGCTC |
| | | | TCAATGACTTCAGTAACTCAGGTTCTGATGTTCTTTAC |
| | | | CAACCACCACAAACCTCTATCACCTCAGCTGTTTTGCA |
| | | | GAGTGGTTTTAGAAAAATGGCATTCCCATCTGGTAAA |
| | | | GTTGAGGGTTGTATGGTACAAGTAACTTGTGGTACAA |
| | | | CTACACTTAACGGTCTTTGGCTTGATGACGTAGTTTAC |
| | | | TGTCCAAGACATGTGATCTGCACCTCTGAAGACATGCT |
| | | | TAACCCTAATTATGAAGATTTACTCATTCGTAAGTCTA |
| | | | ATCATAATTTCTTGGTACAGGCTGGTAATGTTCAACTC |
| | | | AGGGTTATTGGACATTCTATGCAAAATTGTGTACTTAA |
| | | | GCTTAAGGTTGATACAGCCAATCCTAAGACACCTAAG |
| | | | TATAAGTTTGTTCGCATTCAACCAGGACAGACTTTTTC |
| | | | AGTGTTAGCTTGTTACAATGGTTCACCATCTGGTGTTT |
| | | | ACCAATGTGCTATGAGGCCCAATTTCACTATTAAGGGT |
| | | | TCATTCCTTAATGGTTCATGTGGTAGTGTTGGTTTTAA |
| | | | CATAGATTATGACTGTGTCTCTTTTTGTTACATGCACC |
| | | | ATATGGAATTACCAACTGGAGTTCATGCTGGCACAGA |
| | | | CTTAGAAGGTAACTTTTATGGACCTTTTGTTGACAGGC |
| | | | AAACAGCACAAGCAGCTGGTACGGACACAACTATTAC |
| | | | AGTTAATGTTTTAGCTTGGTTGTACGCTGCTGTTATAA |
| | | | ATGGAGACAGGTGGTTTCTCAATCGATTTACCACAACT |
| | | | CTTAATGACTTTAACCTTGTGGCTATGAAGTACAATTA |
| | | | TGAACCTCTAACACAAGACCATGTTGACATACTAGGA |
| | | | CCTCTTTCTGCTCAAACTGGAATTGCCGTTTTAGATAT |
| | | | GTGTGCTTCATTAAAAGAATTACTGCAAAATGGTATG |
| | | | AATGGACGTACCATATTGGGTAGTGCTTTATTAGAAG |
| | | | ATGAATTTACACCTTTTGATGTTGTTAGACAATGCTCA |
| | | | GGTGTTACTTTCCAAAGTGCAGTGAAAAGAACAATCA |
| | | | AGGGTACACACCACTGGTTGTTACTCACAATTTTGACT |
| | | | TCACTTTTAGTTTTAGTCCAGAGTACTCAATGGTCTTT |
| | | | GTTCTTTTTTTTGTATGAAAATGCCTTTTTACCTTTTGC |
| | | | TATGGGTATTATTGCTATGTCTGCTTTTGCAATGATGT |
| | | | TTGTCAAACATAAGCATGCATTTCTCTGTTTGTTTTTGT |
| | | | TACCTTCTCTTGCCACTGTAGCTTATTTTAATATGGTCT |
| | | | ATATGCCTGCTAGTTGGGTGATGCGTATTATGACATGG |
| | | | TTGGATATGGTTGATACTAGTTTGTCTGGTTTTAAGCT |
| | | | AAAAGACTGTGTTATGTATGCATCAGCTGTAGTGTTAC |
| | | | TAATCCTTATGACAGCAAGAACTGTGTATGATGATGG |
| | | | TGCTAGGAGAGTGTGGACACTTATGAATGTCTTGACA |
| | | | CTCGTTTATAAAGTTTATTATGGTAATGCTTTAGATCA |
| | | | AGCCATTTCCATGTGGGCTCTTATAATCTCTGTTACTT |
| | | | CTAACTACTCAGGTGTAGTTACAACTGTCATGTTTTTG |
| | | | GCCAGAGGTATTGTTTTTATGTGTGTTGAGTATTGCCC |
| | | | TATTTTCTTCATAACTGGTAATACACTTCAGTGTATAA |
| | | | TGCTAGTTTATTGTTTCTTAGGCTATTTTTGTACTTGTT |
| | | | ACTTTGGCCTCTTTTGTTTACTCAACCGCTACTTTAGAC |
| | | | TGACTCTTGGTGTTTATGATTACTTAGTTTCTACACAG |
| | | | GAGTTTAGATATATGAATTCACAGGGACTACTCCCAC |
| | | | CCAAGAATAGCATAGATGCCTTCAAACTCAACATTAA |
| | | | ATTGTTGGGTGTTGGTGGCAAACCTTGTATCAAAGTAG |
| | | | CCACTGTACAGTCTAAAATGTCAGATGTAAAGTGCAC |
| | | | ATCAGTAGTCTTACTCTCAGTTTTGCAACAACTCAGAG |
| | | | TAGAATCATCATCTAAATTGTGGGCTCAATGTGTCCAG |
| | | | TTACACAATGACATTCTCTTAGCTAAAGATACTACTGA |
| | | | AGCCTTTGAAAAAATGGTTTCACTACTTTCTGTTTTGC |
| | | | TTTCCATGCAGGGTGCTGTAGACATAAACAAGCTTTGT |
| | | | GAAGAAATGCTGGACAACAGGGCAACCTTACAAGCTA |
| | | | TAGCCTCAGAGTTTAGTTCCCTTCCATCATATGCAGCT |
| | | | TTTGCTACTGCTCAAGAAGCTTATGAGCAGGCTGTTGC |
| | | | TAATGGTGATTCTGAAGTTGTTCTTAAAAAGTTGAAGA |
| | | | AGTCTTTGAATGTGGCTAAATCTGAATTTGACCGTGAT |
| | | | GCAGCCATGCAACGTAAGTTGGAAAAGATGGCTGATC |
| | | | AAGCTATGACCCAAATGTATAAACAGGCTAGATCTGA |
| | | | GGACAAGAGGGCAAAAGTTACTAGTGCTATGCAGACA |
| | | | ATGCTTTTCACTATGCTTAGAAAGTTGGATAATGATGC |
| | | | ACTCAACAACATTATCAACAATGCAAGAGATGGTTGT |
| | | | GTTCCCTTGAACATAATACCTCTTACAACAGCAGCCAA |
| | | | ACTAATGGTTGTCATACCAGACTATAACACATATAAA |
| | | | AATACGTGTGATGGTACAACATTTACTTATGCATCAGC |
| | | | ATTGTGGGAAATCCAACAGGTTGTAGATGCAGATAGT |
| | | | AAAATTGTTCAACTTAGTGAAATTAGTATGGACAATTC |
| | | | ACCTAATTTAGCATGGCCTCTTATTGTAACAGCTTTAA |
| | | | GGGCCAATTCTGCTGTCAAATTACAGAATAATGAGCT |
| | | | TAGTCCTGTTGCACTACGACAGATGTCTTGTGCTGCCG |
| | | | GTACTACACAAACTGCTTGCACTGATGACAATGCGTT |
| | | | AGCTTACTACAACACAACAAAGGGAGGTAGGTTTGTA |
| | | | CTTGCACTGTTATCCGATTTACAGGATTTGAAATGGGC |
| | | | TAGATTCCCTAAGAGTGATGGAACTGGTACTATCTATA |
| | | | CAGAACTGGAACCACCTTGTAGGTTTGTTACAGACAC |
| | | | ACCTAAAGGTCCTAAAGTGAAGTATTTATACTTTATTA |
| | | | AAGGATTAAACAACCTAAATAGAGGTATGGTACTTGG |
| | | | TAGTTTAGCTGCCACAGTACGTCTACAAGCTGGTAATG |
| | | | CAACAGAAGTGCCTGCCAATTCAACTGTATTATCTTTC |
| | | | TGTGCTTTTGCTGTAGATGCTGCTAAAGCTTACAAAGA |
| | | | TTATCTAGCTAGTGGGGGACAACCAATCACTAATTGT |
| | | | GTTAAGATGTTGTGTACACACACTGGTACTGGTCAGG |
| | | | CAATAACAGTTACACCGGAAGCCAATATGGATCAAGA |
| | | | ATCCTTTGGTGGTGCATCGTGTTGTCTGTACTGCCGTT |
| | | | GCCACATAGATCATCCAAATCCTAAAGGATTTTGTGA |
| | | | CTTAAAAGGTAAGTATGTACAAATACCTACAACTTGT |
| | | | GCTAATGACCCTGTGGGTTTTACACTTAAAAACACAGT |
| | | | CTGTACCGTCTGCGGTATGTGGAAAGGTTATGGCTGTA |
| | | | GTTGTGATCAACTCCGCGAACCCATGCTTCAGTCAGCT |
| | | | GATGCACAATCGTTTTTAAACGGGTTTGCGGTGTAAGT |
| | | | GCAGCCCGTCTTACACCGTGCGGCACAGGCACTAGTA |
| | | | CTGATGTCGTATACAGGGCTTTTGACATCTACAATGAT |
| | | | AAAGTAGCTGGTTTTGCTAAATTCCTAAAAACTAATTG |
| | | | TTGTCGCTTCCAAGAAAAGGACGAAGATGACAATTTA |
| | | | ATTGATTCTTACTTTGTAGTTAAGAGACACACTTTCTC |
| | | | TAACTACCAACATGAAGAAACAATTTATAATTTACTTA |
| | | | AGGATTGTCCAGCTGTTGCTAAACATGACTTCTTTAAG |
| | | | TTTAGAATAGACGGTGACATGGTACCACATATATCAC |
| | | | GTCAACGTCTTACTAAATACACAATGGCAGACCTCGT |
| | | | CTATGCTTTAAGGCATTTTGATGAAGGTAATTGTGACA |
| | | | CATTAAAAGAAATACTTGTCACATACAATTGTTGTGAT |
| | | | GATGATTATTTCAATAAAAAGGACTGGTATGATTTTGT |
| | | | AGAAAACCCAGATATATTACGCGTATACGCCAACTTA |
| | | | GGTGAACGTGTACGCCAAGCTTTGTTAAAAACAGTAC |
| | | | AATTCTGTGATGCCATGCGAAATGCTGGTATTGTTGGT |
| | | | GTACTGACATTAGATAATCAAGATCTCAATGGTAACT |
| | | | GGTATGATTTCGGTGATTTCATACAAACCACGCCAGGT |
| | | | AGTGGAGTTCCTGTTGTAGATTCTTATTATTCATTGTT |
| | | | AATGCCTATATTAACCTTGACCAGGGCTTTAACTGCAG |
| | | | AGTCACATGTTGACACTGACTTAACAAAGCCTTACATT |
| | | | AAGTGGGATTTGTTAAAATATGACTTCACGGAAGAGA |
| | | | GGTTAAAACTCTTTGACCGTTATTTTAAATATTGGGAT |
| | | | CAGACATACCACCCAAATTGTGTTAACTGTTTGGATGA |
| | | | CAGATGCATTCTGCATTGTGCAAACTTTAATGTTTTAT |
| | | | TCTCTACAGTGTTCCCACCTACAAGTTTTGGACCACTA |
| | | | GTGAGAAAAATATTTGTTGATGGTGTTCCATTTGTAGT |
| | | | TTCAACTGGATACCACTTCAGAGAGCTAGGTGTTGTAC |
| | | | ATAATCAGGATGTAAACTTACATAGCTCTAGACTTAGT |
| | | | TTTAAGGAATTACTTGTGTATGCTGCTGACCCTGCTAT |
| | | | GCACGCTGCTTCTGGTAATCTATTACTAGATAAACGCA |
| | | | CTACGTGCTTTTCAGTAGCTGCACTTACTAACAATGTT |
| | | | GCTTTTCAAACTGTCAAACCCGGTAATTTTAACAAAGA |
| | | | CTTCTATGACTTTGCTGTGTCTAAGGGTTTCTTTAAGG |
| | | | AAGGAAGTTCTGTTGAATTAAAACACTTCTTCTTTGCT |
| | | | CAGGATGGTAATGCTGCTATCAGCGATTATGACTACT |
| | | | ATCGTTATAATCTACCAACAATGTGTGATATCAGACA |
| | | | ACTACTATTTGTAGTTGAAGTTGTTGATAAGTACTTTG |
| | | | ATTGTTACGATGGTGGCTGTATTAATGCTAACCAAGTC |
| | | | ATCGTCAACAACCTAGACAAATCAGCTGGTTTTCCATT |
| | | | TAATAAATGGGGTAAGGCTAGACTTTATTATGATTCA |
| | | | ATGAGTTATGAGGATCAAGATGCACTTTTCGCATATAC |
| | | | AAAACGTAATGTCATCCCTACTATAACTCAAATGAAT |
| | | | CTTAAGTATGCCATTAGTGCAAAGAATAGAGCTCGCA |
| | | | CCGTAGCTGGTGTCTCTATCTGTAGTACTATGACCAAT |
| | | | AGACAGTTTCATCAAAAATTATTGAAATCAATAGCCG |
| | | | CCACTAGAGGAGCTACTGTAGTAATTGGAACAAGCAA |
| | | | ATTCTATGGTGGTTGGCACAACATGTTAAAAACTGTTT |
| | | | ATAGTGATGTAGAAAACCCTCACCTTATGGGTTGGGA |
| | | | TTATCCTAAATGTGATAGAGCCATGCCTAACATGCTTA |
| | | | GAATTATGGCCTCACTTGTTCTTGCTCGCAAACATACA |
| | | | ACGTGTTGTAGCTTGTCACACCGTTTCTATAGATTAGC |
| | | | TAATGAGTGTGCTCAAGTATTGAGTGAAATGGTCATG |
| | | | TGTGGCGGTTCACTATATGTTAAACCAGGTGGAACCTC |
| | | | ATCAGGAGATGCCACAACTGCTTATGCTAATAGTGTTT |
| | | | TTAACATTTGTCAAGCTGTCACGGCCAATGTTAATGCA |
| | | | CTTTTATCTACTGATGGTAACAAAATTGCCGATAAGTA |
| | | | TGTCCGCAATTTACAACACAGACTTTATGAGTGTCTCT |
| | | | ATAGAAATAGAGATGTTGACACAGACTTTGTGAATGA |
| | | | GTTTTACGCATATTTGCGTAAACATTTCTCAATGATGA |
| | | | TACTCTCTGACGATGCTGTTGTGTGTTTCAATAGCACT |
| | | | TATGCATCTCAAGGTCTAGTGGCTAGCATAAAGAACT |
| | | | TTAAGTCAGTTCTTTATTATCAAAACAATGTTTTTATG |
| | | | TCTGAAGCAAAATGTTGGACTGAGACTGACCTTACTA |
| | | | AAGGACCTCATGAATTTTGCTCTCAACATACAATGCTA |
| | | | GTTAAACAGGGTGATGATTATGTGTACCTTCCTTACCC |
| | | | AGATCCATCAAGAATCCTAGGGGCCGGCTGTTTTGTA |
| | | | GATGATATCGTAAAAACAGATGGTACACTTATGATTG |
| | | | AACGGTTCGTGTCTTTAGCTATAGATGCTTACCCACTT |
| | | | ACTAAACATCCTAATCAGGAGTATGCTGATGTCTTTCA |
| | | | TTTGTACTTACAATACATAAGAAAGCTACATGATGAG |
| | | | TTAACAGGACACATGTTAGACATGTATTCTGTTATGCT |
| | | | TACTAATGATAACACTTCAAGGTATTGGGAACCTGAG |
| | | | TTTTATGAGGCTATGTACACACCGCATACAGTCTTACA |
| | | | GGCTGTTGGGGCTTGTGTTCTTTGCAATTCACAGACTT |
| | | | CATTAAGATGTGGTGCTTGCATACGTAGACCATTCTTA |
| | | | TGTTGTAAATGCTGTTACGACCATGTCATATCAACATC |
| | | | ACATAAATTAGTCTTGTCTGTTAATCCGTATGTTTGCA |
| | | | ATGCTCCAGGTTGTGATGTCACAGATGTGACTCAACTT |
| | | | TACTTAGGAGGTATGAGCTATTATTGTAAATCACATAA |
| | | | ACCACCCATTAGTTTTCCATTGTGTGCTAATGGACAAG |
| | | | TTTTTGGTTTATATAAAAATACATGTGTTGGTAGCGAT |
| | | | AATGTTACTGACTTTAATGCAATTGCAACATGTGACTG |
| | | | GACAAATGCTGGTGATTACATTTTAGCTAACACCTGTA |
| | | | CTGAAAGACTCAAGCTTTTTGCAGCAGAAACGCTCAA |
| | | | AGCTACTGAGGAGACATTTAAACTGTCTTATGGTATTG |
| | | | CTACTGTACGTGAAGTGCTGTCTGACAGAGAATTACA |
| | | | TCTTTCATGGGAAGTTGGTAAACCTAGACCACCACTTA |
| | | | ACCGAAATTATGTCTTTACTGGTTATCGTGTAACTAAA |
| | | | AACAGTAAAGTACAAATAGGAGAGTACACCTTTGAAA |
| | | | AAGGTGACTATGGTGATGCTGTTGTTTACCGAGGTAC |
| | | | AACAACTTACAAATTAAATGTTGGTGATTATTTTGTGC |
| | | | TGACATCACATACAGTAATGCCATTAAGTGCACCTAC |
| | | | ACTAGTGCCACAAGAGCACTATGTTAGAATTACTGGC |
| | | | TTATACCCAACACTCAATATCTCAGATGAGTTTTCTAG |
| | | | CAATGTTGCAAATTATCAAAAGGTTGGTATGCAAAAG |
| | | | TATTCTACACTCCAGGGACCACCTGGTACTGGTAAGA |
| | | | GTCATTTTGCTATTGGCCTAGCTCTCTACTACCCTTCTG |
| | | | CTCGCATAGTGTATACAGCTTGCTCTCATGCCGCTGTT |
| | | | GATGCACTATGTGAGAAGGCATTAAAATATTTGCCTA |
| | | | TAGATAAATGTAGTAGAATTATACCTGCACGTGCTCGT |
| | | | GTAGAGTGTTTTGATAAATTCAAAGTGAATTCAACATT |
| | | | AGAACAGTATGTCTTTTGTACTGTAAATGCATTGCCTG |
| | | | AGACGACAGCAGATATAGTTGTCTTTGATGAAATTTC |
| | | | AATGGCCACAAATTATGATTTGAGTGTTGTCAATGCCA |
| | | | GATTACGTGCTAAGCACTATGTGTACATTGGCGACCCT |
| | | | GCTCAATTACCTGCACCACGCACATTGCTAACTAAGG |
| | | | GCACACTAGAACCAGAATATTTCAATTCAGTGTGTAG |
| | | | ACTTATGAAAACTATAGGTCCAGACATGTTCCTCGGA |
| | | | ACTTGTCGGCGTTGTCCTGCTGAAATTGTTGACACTGT |
| | | | GAGTGCTTTGGTTTATGATAATAAGCTTAAAGCACATA |
| | | | AAGACAAATCAGCTCAATGCTTTAAAATGTTTTATAA |
| | | | GGGTGTTATCACGCATGATGTTTCATCTGCAATTAACA |
| | | | GGCCACAAATAGGCGTGGTAAGAGAATTCCTTACACG |
| | | | TAACCCTGCTTGGAGAAAAGCTGTCTTTATTTCACCTT |
| | | | ATAATTCACAGAATGCTGTAGCCTCAAAGATTTTGGG |
| | | | ACTACCAACTCAAACTGTTGATTCATCACAGGGCTCA |
| | | | GAATATGACTATGTCATATTCACTCAAACCACTGAAA |
| | | | CAGCTCACTCTTGTAATGTAAACAGATTTAATGTTGCT |
| | | | ATTACCAGAGCAAAAGTAGGCATACTTTGCATAATGT |
| | | | CTGATAGAGACCTTTATGACAAGTTGCAATTTACAAGT |
| | | | CTTGAAATTCCACGTAGGAATGTGGCAACTTTACAAG |
| | | | CTGAAAATGTAACAGGACTCTTTAAAGATTGTAGTAA |
| | | | GGTAATCACTGGGTTACATCCTACACAGGCACCTACA |
| | | | CACCTCAGTGTTGACACTAAATTCAAAACTGAAGGTTT |
| | | | ATGTGTTGACATACCTGGCATACCTAAGGACATGACC |
| | | | TATAGAAGACTCATCTCTATGATGGGTTTTAAAATGAA |
| | | | TTATCAAGTTAATGGTTACCCTAACATGTTTATCACCC |
| | | | GCGAAGAAGCTATAAGACATGTACGTGCATGGATTGG |
| | | | CTTCGATGTCGAGGGGTGTCATGCTACTAGAGAAGCT |
| | | | GTTGGTACCAATTTACCTTTACAGCTAGGTTTTTCTAC |
| | | | AGGTGTTAACCTAGTTGCTGTACCTACAGGTTATGTTG |
| | | | ATACACCTAATAATACAGATTTTTCCAGAGTTAGTGCT |
| | | | AAACCACCGCCTGGAGATCAATTTAAACACCTCATAC |
| | | | CACTTATGTACAAAGGACTTCCTTGGAATGTAGTGCGT |
| | | | ATAAAGATTGTACAAATGTTAAGTGACACACTTAAAA |
| | | | ATCTCTCTGACAGAGTCGTATTTGTCTTATGGGCACAT |
| | | | GGCTTTGAGTTGACATCTATGAAGTATTTTGTGAAAAT |
| | | | AGGACCTGAGCGCACCTGTTGTCTATGTGATAGACGT |
| | | | GCCACATGCTTTTCCACTGCTTCAGACACTTATGCCTG |
| | | | TTGGCATCATTCTATTGGATTTGATTACGTCTATAATC |
| | | | CGTTTATGATTGATGTTCAACAATGGGGTTTTACAGGT |
| | | | AACCTACAAAGCAACCATGATCTGTATTGTCAAGTCC |
| | | | ATGGTAATGCACATGTAGCTAGTTGTGATGCAATCAT |
| | | | GACTAGGTGTCTAGCTGTCCACGAGTGCTTTGTTAAGC |
| | | | GTGTTGACTGGACTATTGAATATCCTATAATTGGTGAT |
| | | | GAACTGAAGATTAATGCGGCTTGTAGAAAGGTTCAAC |
| | | | ACATGGTTGTTAAAGCTGCATTATTAGCAGACAAATTC |
| | | | CCAGTTCTTCACGACATTGGTAACCCTAAAGCTATTAA |
| | | | GTGTGTACCTCAAGCTGATGTAGAATGGAAGTTCTAT |
| | | | GATGCACAGCCTTGTAGTGACAAAGCTTATAAAATAG |
| | | | AAGAATTATTCTATTCTTATGCCACACATTCTGACAAA |
| | | | TTCACAGATGGTGTATGCCTATTTTGGAATTGCAATGT |
| | | | CGATAGATATCCTGCTAATTCCATTGTTTGTAGATTTG |
| | | | ACACTAGAGTGCTATCTAACCTTAACTTGCCTGGTTGT |
| | | | GATGGTGGCAGTTTGTATGTAAATAAACATGCATTCC |
| | | | ACACACCAGCTTTTGATAAAAGTGCTTTTGTTAATTTA |
| | | | AAACAATTACCATTTTTCTATTACTCTGACAGTCCATG |
| | | | TGAGTCTCATGGAAAACAAGTAGTGTCAGATATAGAT |
| | | | TATGTACCACTAAAGTCTGCTACGTGTATAACACGTTG |
| | | | CAATTTAGGTGGTGCTGTCTGTAGACATCATGCTAATG |
| | | | AGTACAGATTGTATCTCGATGCTTATAACATGATGATC |
| | | | TCAGCTGGCTTTAGCTTGTGGGTTTACAAACAATTTGA |
| | | | TACTTATAACCTCTGGAACACTTTTACAAGACTTCAGA |
| | | | GTTTAGAAAATGTGGCTTTTAATGTTGTAAATAAGGG |
| | | | ACACTTTGATGGACAACAGGGTGAAGTACCAGTTTCT |
| | | | ATCATTAATAACACTGTTTACACAAAAGTTGATGGTGT |
| | | | TGATGTAGAATTGTTTGAAAATAAAACAACATTACCT |
| | | | GTTAATGTAGCATTTGAGCTTTGGGCTAAGCGCAACAT |
| | | | TAAACCAGTACCAGAGGTGAAAATACTCAATAATTTG |
| | | | GGTGTGGACATTGCTGCTAATACTGTGATCTGGGACTA |
| | | | CAAAAGAGATGCTCCAGCACATATATCTACTATTGGT |
| | | | GTTTGTTCTATGACTGACATAGCCAAGAAACCAACTG |
| | | | AAACGATTTGTGCACCACTCACTGTCTTTTTTGATGGT |
| | | | AGAGTTGATGGTCAAGTAGACTTATTTAGAAATGCCC |
| | | | GTAATGGTGTTCTTATTACAGAAGGTAGTGTTAAAGGT |
| | | | TTACAACCATCTGTAGGTCCCAAACAAGCTAGTCTTAA |
| | | | TGGAGTCACATTAATTGGAGAAGCCGTAAAAACACAG |
| | | | TTCAATTATTATAAGAAAGTTGATGGTGTTGTCCAACA |
| | | | ATTACCTGAAACTTACTTTACTCAGAGTAGAAATTTAC |
| | | | AAGAATTTAAACCCAGGAGTCAAATGGAAATTGATTT |
| | | | CTTAGAATTAGCTATGGATGAATTCATTGAACGGTATA |
| | | | AATTAGAAGGCTATGCCTTCGAACATATCGTTTATGGA |
| | | | GATTTTAGTCATAGTCAGTTAGGTGGTTTACATCTACT |
| | | | GATTGGACTAGCTAAACGTTTTAAGGAATCACCTTTTG |
| | | | AATTAGAAGATTTTATTCCTATGGACAGTACAGTTAAA |
| | | | AACTATTTCATAACAGATGCGCAAACAGGTTCATCTA |
| | | | AGTGTGTGTGTTCTGTTATTGATTTATTACTTGATGATT |
| | | | TTGTTGAAATAATAAAATCCCAAGATTTATCTGTAGTT |
| | | | TCTAAGGTTGTCAAAGTGACTATTGACTATACAGAAA |
| | | | TTTCATTTATGCTTTGGTGTAAAGATGGCCATGTAGAA |
| | | | ACATTTTACCCAAAATTACAATCTAGTCAAGCGTGGC |
| | | | AACCGGGTGTTGCTATGCCTAATCTTTACAAAATGCAA |
| | | | AGAATGCTATTAGAAAAGTGTGACCTTCAAAATTATG |
| | | | GTGATAGTGCAACATTACCTAAAGGCATAATGATGAA |
| | | | TGTCGCAAAATATACTCAACTGTGTCAATATTTAAACA |
| | | | CATTAACATTAGCTGTACCCTATAATATGAGAGTTATA |
| | | | CATTTTGGTGCTGGTTCTGATAAAGGAGTTGCACCAGG |
| | | | TACAGCTGTTTTAAGACAGTGGTTGCCTACGGGTACGC |
| | | | TGCTTGTCGATTCAGATCTTAATGACTTTGTCTCTGAT |
| | | | GCAGATTCAACTTTGATTGGTGATTGTGCAACTGTACA |
| | | | TACAGCTAATAAATGGGATCTCATTATTAGTGATATGT |
| | | | ACGACCCTAAGACTAAAAATGTTACAAAAGAAAATGA |
| | | | CTCTAAAGAGGGTTTTTTCACTTACATTTGTGGGTTTA |
| | | | TACAACAAAAGCTAGCTCTTGGAGGTTCCGTGGCTAT |
| | | | AAAGATAACAGAACATTCTTGGAATGCTGATCTTTAT |
| | | | AAGCTCATGGGACACTTCGCATGGTGGACAGCCTTTG |
| | | | TTACTAATGTGAATGCGTCATCATCTGAAGCATTTTTA |
| | | | ATTGGATGTAATTATCTTGGCAAACCACGCGAACAAA |
| | | | TAGATGGTTATGTCATGCATGCAAATTACATATTTTGG |
| | | | AGGAATACAAATCCAATTCAGTTGTCTTCCTATTCTTT |
| | | | ATTTGACATGAGTAAATTTCCCCTTAAATTAAGGGGTA |
| | | | CTGCTGTTATGTCTTTAAAAGAAGGTCAAATCAATGAT |
| | | | ATGATTTTATCTCTTCTTAGTAAAGGTAGACTTATAAT |
| | | | TAGAGAAAACAACAGAGTTGTTATTTCTAGTGATGTTC |
| | | | TTGTTAACAACTA |
| S | 21563 | 25384 | ATGTTTGTTTTTCTTGTTTTATTGCCACTAGTCTCTAGT |
| (SEQ ID | | | CAGTGTGTTAATCTTACAACCAGAACTCAATTACCCCC |
| NO: 4) | | | TGCATACACTAATTCTTTCACACGTGGTGTTTATTACC |
| | | | CTGACAAAGTTTTCAGATCCTCAGTTTTACATTCAACT |
| | | | CAGGACTTGTTCTTACCTTTCTTTTCCAATGTTACTTGG |
| | | | TTCCATGCTATACATGTCTCTGGGACCAATGGTACTAA |
| | | | GAGGTTTGATAACCCTGTCCTACCATTTAATGATGGTG |
| | | | TTTATTTTGCTTCCACTGAGAAGTCTAACATAATAAGA |
| | | | GGCTGGATTTTTGGTACTACTTTAGATTCGAAGACCCA |
| | | | GTCCCTACTTATTGTTAATAACGCTACTAATGTTGTTA |
| | | | TTAAAGTCTGTGAATTTCAATTTTGTAATGATCCATTT |
| | | | TTGGGTGTTTATTACCACAAAAACAACAAAAGTTGGA |
| | | | TGGAAAGTGAGTTCAGAGTTTATTCTAGTGCGAATAA |
| | | | TTGCACTTTTGAATATGTCTCTCAGCCTTTTCTTATGGA |
| | | | CCTTGAAGGAAAACAGGGTAATTTCAAAAATCTTAGG |
| | | | GAATTTGTGTTTAAGAATATTGATGGTTATTTTAAAAT |
| | | | ATATTCTAAGCACACGCCTATTAATTTAGTGCGTGATC |
| | | | TCCCTCAGGGTTTTTCGGCTTTAGAACCATTGGTAGAT |
| | | | TTGCCAATAGGTATTAACATCACTAGGTTTCAAACTTT |
| | | | ACTTGCTTTACATAGAAGTTATTTGACTCCTGGTGATT |
| | | | CTTCTTCAGGTTGGACAGCTGGTGCTGCAGCTTATTAT |
| | | | GTGGGTTATCTTCAACCTAGGACTTTTCTATTAAAATA |
| | | | TAATGAAAATGGAACCATTACAGATGCTGTAGACTGT |
| | | | GCACTTGACCCTCTCTCAGAAACAAAGTGTACGTTGA |
| | | | AATCCTTCACTGTAGAAAAAGGAATCTATCAAACTTCT |
| | | | AACTTTAGAGTCCAACCAACAGAATCTATTGTTAGATT |
| | | | TCCTAATATTACAAACTTGTGCCCTTTTGGTGAAGTTT |
| | | | TTAACGCCACCAGATTTGCATCTGTTTATGCTTGGAAC |
| | | | AGGAAGAGAATCAGCAACTGTGTTGCTGATTATTCTG |
| | | | TCCTATATAATTCCGCATCATTTTCCACTTTTAAGTGTT |
| | | | ATGGAGTGTCTCCTACTAAATTAAATGATCTCTGCTTT |
| | | | ACTAATGTCTATGCAGATTCATTTGTAATTAGAGGTGA |
| | | | TGAAGTCAGACAAATCGCTCCAGGGCAAACTGGAAAG |
| | | | ATTGCTGATTATAATTATAAATTACCAGATGATTTTAC |
| | | | AGGCTGCGTTATAGCTTGGAATTCTAACAATCTTGATT |
| | | | CTAAGGTTGGTGGTAATTATAATTACCTGTATAGATTG |
| | | | TTTAGGAAGTCTAATCTCAAACCTTTTGAGAGAGATAT |
| | | | TTCAACTGAAATCTATCAGGCCGGTAGCACACCTTGTA |
| | | | ATGGTGTTGAAGGTTTTAATTGTTACTTTCCTTTACAA |
| | | | TCATATGGTTTCCAACCCACTAATGGTGTTGGTTACCA |
| | | | ACCATACAGAGTAGTAGTACTTTCTTTTGAACTTCTAC |
| | | | ATGCACCAGCAACTGTTTGTGGACCTAAAAAGTCTAC |
| | | | TAATTTGGTTAAAAACAAATGTGTCAATTTCAACTTCA |
| | | | ATGGTTTAACAGGCACAGGTGTTCTTACTGAGTCTAAC |
| | | | AAAAAGTTTCTGCCTTTCCAACAATTTGGCAGAGACAT |
| | | | TGCTGACACTACTGATGCTGTCCGTGATCCACAGACAC |
| | | | TTGAGATTCTTGACATTACACCATGTTCTTTTGGTGGT |
| | | | GTCAGTGTTATAACACCAGGAACAAATACTTCTAACC |
| | | | AGGTTGCTGTTCTTTATCAGGATGTTAACTGCACAGAA |
| | | | GTCCCTGTTGCTATTCATGCAGATCAACTTACTCCTAC |
| | | | TTGGCGTGTTTATTCTACAGGTTCTAATGTTTTTCAAA |
| | | | CACGTGCAGGCTGTTTAATAGGGGCTGAACATGTCAA |
| | | | CAACTCATATGAGTGTGACATACCCATTGGTGCAGGT |
| | | | ATATGCGCTAGTTATCAGACTCAGACTAATTCTCCTCG |
| | | | GCGGGCACGTAGTGTAGCTAGTCAATCCATCATTGCCT |
| | | | ACACTATGTCACTTGGTGCAGAAAATTCAGTTGCTTAC |
| | | | TCTAATAACTCTATTGCCATACCCACAAATTTTACTAT |
| | | | TAGTGTTACCACAGAAATTCTACCAGTGTCTATGACCA |
| | | | AGACATCAGTAGATTGTACAATGTACATTTGTGGTGAT |
| | | | TCAACTGAATGCAGCAATCTTTTGTTGCAATATGGCAG |
| | | | TTTTTGTACACAATTAAACCGTGCTTTAACTGGAATAG |
| | | | CTGTTGAACAAGACAAAAACACCCAAGAAGTTTTTGC |
| | | | ACAAGTCAAACAAATTTACAAAACACCACCAATTAAA |
| | | | GATTTTGGTGGTTTTAATTTTTCACAAATATTACCAGA |
| | | | TCCATCAAAACCAAGCAAGAGGTCATTTATTGAAGAT |
| | | | CTACTTTTCAACAAAGTGACACTTGCAGATGCTGGCTT |
| | | | CATCAAACAATATGGTGATTGCCTTGGTGATATTGCTG |
| | | | CTAGAGACCTCATTTGTGCACAAAAGTTTAACGGCCTT |
| | | | ACTGTTTTGCCACCTTTGCTCACAGATGAAATGATTGC |
| | | | TCAATACACTTCTGCACTGTTAGCGGGTACAATCACTT |
| | | | CTGGTTGGACCTTTGGTGCAGGTGCTGCATTACAAATA |
| | | | CCATTTGCTATGCAAATGGCTTATAGGTTTAATGGTAT |
| | | | TGGAGTTACACAGAATGTTCTCTATGAGAACCAAAAA |
| | | | TTGATTGCCAACCAATTTAATAGTGCTATTGGCAAAAT |
| | | | TCAAGACTCACTTTCTTCCACAGCAAGTGCACTTGGAA |
| | | | AACTTCAAGATGTGGTCAACCAAAATGCACAAGCTTT |
| | | | AAACACGCTTGTTAAACAACTTAGCTCCAATTTTGGTG |
| | | | CAATTTCAAGTGTTTTAAATGATATCCTTTCACGTCTT |
| | | | GACAAAGTTGAGGCTGAAGTGCAAATTGATAGGTTGA |
| | | | TCACAGGCAGACTTCAAAGTTTGCAGACATATGTGAC |
| | | | TCAACAATTAATTAGAGCTGCAGAAATCAGAGCTTCT |
| | | | GCTAATCTTGCTGCTACTAAAATGTCAGAGTGTGTACT |
| | | | TGGACAATCAAAAAGAGTTGATTTTTGTGGAAAGGGC |
| | | | TATCATCTTATGTCCTTCCCTCAGTCAGCACCTCATGG |
| | | | TGTAGTCTTCTTGCATGTGACTTATGTCCCTGCACAAG |
| | | | AAAAGAACTTCACAACTGCTCCTGCCATTTGTCATGAT |
| | | | GGAAAAGCACACTTTCCTCGTGAAGGTGTCTTTGTTTC |
| | | | AAATGGCACACACTGGTTTGTAACACAAAGGAATTTT |
| | | | TATGAACCACAAATCATTACTACAGACAACACATTTG |
| | | | TGTCTGGTAACTGTGATGTTGTAATAGGAATTGTCAAC |
| | | | AACACAGTTTATGATCCTTTGCAACCTGAATTAGACTC |
| | | | ATTCAAGGAGGAGTTAGATAAATATTTTAAGAATCAT |
| | | | ACATCACCAGATGTTGATTTAGGTGACATCTCTGGCAT |
| | | | TAATGCTTCAGTTGTAAACATTCAAAAAGAAATTGAC |
| | | | CGCCTCAATGAGGTTGCCAAGAATTTAAATGAATCTCT |
| | | | CATCGATCTCCAAGAACTTGGAAAGTATGAGCAGTAT |
| | | | ATAAAATGGCCATGGTACATTTGGCTAGGTTTTATAGC |
| | | | TGGCTTGATTGCCATAGTAATGGTGACAATTATGCTTT |
| | | | GCTGTATGACCAGTTGCTGTAGTTGTCTCAAGGGCTGT |
| | | | TGTTCTTGTGGATCCTGCTGCAAATTTGATGAAGACGA |
| | | | CTCTGAGCCAGTGCTCAAAGGAGTCAAATTACATTAC |
| | | | ACATA |
| 3a | 25393 | 26220 | ATGGATTTGTTTATGAGAATCTTCACAATTGGAACTGT |
| (SEQ ID | | | AACTTTGAAGCAAGGTGAAATCAAGGATGCTACTCCT |
| NO: 5) | | | TCAGATTTTGTTCGCGCTACTGCAACGATACCGATACA |
| | | | AGCCTCACTCCCTTTCGGATGGCTTATTGTTGGCGTTG |
| | | | CACTTCTTGCTGTTTTTCAGAGCGCTTCCAAAATCATA |
| | | | ACCCTCAAAAAGAGATGGCAACTAGCACTCTCCAAGG |
| | | | GTGTTCACTTTGTTTGCAACTTGCTGTTGTTGTTTGTAA |
| | | | CAGTTTACTCACACCTTTTGCTCGTTGCTGCTGGCCTT |
| | | | GAAGCCCCTTTTCTCTATCTTTATGCTTTAGTCTACTTC |
| | | | TTGCAGAGTATAAACTTTGTAAGAATAATAATGAGGC |
| | | | TTTGGCTTTGCTGGAAATGCCGTTCCAAAAACCCATTA |
| | | | CTTTATGATGCCAACTATTTTCTTTGCTGGCATACTAA |
| | | | TTGTTACGACTATTGTATACCTTACAATAGTGTAACTT |
| | | | CTTCAATTGTCATTACTTCAGGTGATGGCACAACAAGT |
| | | | CCTATTTCTGAACATGACTACCAGATTGGTGGTTATAC |
| | | | TGAAAAATGGGAATCTGGAGTAAAAGACTGTGTTGTA |
| | | | TTACACAGTTACTTCACTTCAGACTATTACCAGCTGTA |
| | | | CTCAACTCAATTGAGTACAGACACTGGTGTTGAACAT |
| | | | GTTACCTTCTTCATCTACAATAAAATTGTTGATGAGCC |
| | | | TGAAGAACATGTCCAAATTCACACAATCGACGGTTCA |
| | | | TCCGGAGTTGTTAATCCAGTAATGGAACCAATTTATGA |
| | | | TGAACCGACGACGACTACTAGCGTGCCTTTGTA |
| E | 26245 | 26472 | ATGTACTCATTCGTTTCGGAAGAGACAGGTACGTTAAT |
| (SEQ ID | | | AGTTAATAGCGTACTTCTTTTTCTTGCTTTCGTGGTATT |
| NO: 6) | | | CTTGCTAGTTACACTAGCCATCCTTACTGCGCTTCGAT |
| | | | TGTGTGCGTACTGCTGCAATATTGTTAACGTGAGTCTT |
| | | | GTAAAACCTTCTTTTTACGTTTACTCTCGTGTTAAAAA |
| | | | TCTGAATTCTTCTAGAGTTCCTGATCTTCTGGTCTA |
| M | 26523 | 27191 | ATGGCAGATTCCAACGGTACTATTACCGTTGAAGAGC |
| (SEQ ID | | | TTAAAAAGCTCCTTGAACAATGGAACCTAGTAATAGG |
| NO: 7) | | | TTTCCTATTCCTTACATGGATTTGTCTTCTACAATTTGC |
| | | | CTATGCCAACAGGAATAGGTTTTTGTATATAATTAAGT |
| | | | TAATTTTCCTCTGGCTGTTATGGCCAGTAACTTTAGCT |
| | | | TGTTTTGTGCTTGCTGCTGTTTACAGAATAAATTGGAT |
| | | | CACCGGTGGAATTGCTATCGCAATGGCTTGTCTTGTAG |
| | | | GCTTGATGTGGCTCAGCTACTTCATTGCTTCTTTCAGA |
| | | | CTGTTTGCGCGTACGCGTTCCATGTGGTCATTCAATCC |
| | | | AGAAACTAACATTCTTCTCAACGTGCCACTCCATGGCA |
| | | | CTATTCTGACCAGACCGCTTCTAGAAAGTGAACTCGTA |
| | | | ATCGGAGCTGTGATCCTTCGTGGACATCTTCGTATTGC |
| | | | TGGACACCATCTAGGACGCTGTGACATCAAGGACCTG |
| | | | CCTAAAGAAATCACTGTTGCTACATCACGAACGCTTTC |
| | | | TTATTACAAATTGGGAGCTTCGCAGCGTGTAGCAGGT |
| | | | GACTCAGGTTTTGCTGCATACAGTCGCTACAGGATTGG |
| | | | CAACTATAAATTAAACACAGACCATTCCAGTAGCAGT |
| | | | GACAATATTGCTTTGCTTGTACAGTA |
| 7a | 27394 | 27759 | ATGAAAATTATTCTTTTCTTGGCACTGATAACACTCGC |
| (SEQ ID | | | TACTTGTGAGCTTTATCACTACCAAGAGTGTGTTAGAG |
| NO: 8) | | | GTACAACAGTACTTTTAAAAGAACCTTGCTCTTCTGGA |
| | | | ACATACGAGGGCAATTCACCATTTCATCCTCTAGCTGA |
| | | | TAACAAATTTGCACTGACTTGCTTTAGCACTCAATTTG |
| | | | CTTTTGCTTGTCCTGACGGCGTAAAACACGTCTATCAG |
| | | | TTACGTGCCAGATCAGTTTCACCTAAACTGTTCATCAG |
| | | | ACAAGAGGAAGTTCAAGAACTTTACTCTCCAATTTTTC |
| | | | TTATTGTTGCGGCAATAGTGTTTATAACACTTTGCTTC |
| | | | ACACTCAAAAGAAAGACAGAATG |
| 8a | 27894 | 28259 | ATGAAATTTCTTGTTTTCTTAGGAATCATCACAACTGT |
| (SEQ ID | | | AGCTGCATTTCACCAAGAATGTAGTTTACAGTCATGTA |
| NO: 9) | | | CTCAACATCAACCATATGTAGTTGATGACCCGTGTCCT |
| | | | ATTCACTTCTATTCTAAATGGTATATTAGAGTAGGAGC |
| | | | TAGAAAATCAGCACCTTTAATTGAATTGTGCGTGGAT |
| | | | GAGGCTGGTTCTAAATCACCCATTCAGTACATCGATAT |
| | | | CGGTAATTATACAGTTTCCTGTTTACCTTTTACAATTA |
| | | | ATTGCCAGGAACCTAAATTGGGTAGTCTTGTAGTGCGT |
| | | | TGTTCGTTCTATGAAGACTTTTTAGAGTATCATGACGT |
| | | | TCGTGTTGTTTTAGATTTCATCTA |
| N | 28274 | 29533 | ATGTCTGATAATGGACCCCAAAATCAGCGAAATGCAC |
| (SEQ ID | | | CCCGCATTACGTTTGGTGGACCCTCAGATTCAACTGGC |
| NO: 10) | | | AGTAACCAGAATGGAGAACGCAGTGGGGCGCGATCA |
| | | | AAACAACGTCGGCCCCAAGGTTTACCCAATAATACTG |
| | | | CGTCTTGGTTCACCGCTCTCACTCAACATGGCAAGGAA |
| | | | GACCTTAAATTCCCTCGAGGACAAGGCGTTCCAATTA |
| | | | ACACCAATAGCAGTCCAGATGACCAAATTGGCTACTA |
| | | | CCGAAGAGCTACCAGACGAATTCGTGGTGGTGACGGT |
| | | | AAAATGAAAGATCTCAGTCCAAGATGGTATTTCTACT |
| | | | ACCTAGGAACTGGGCCAGAAGCTGGACTTCCCTATGG |
| | | | TGCTAACAAAGACGGCATCATATGGGTTGCAACTGAG |
| | | | GGAGCCTTGAATACACCAAAAGATCACATTGGCACCC |
| | | | GCAATCCTGCTAACAATGCTGCAATCGTGCTACAACTT |
| | | | CCTCAAGGAACAACATTGCCAAAAGGCTTCTACGCAG |
| | | | AAGGGAGCAGAGGCGGCAGTCAAGCCTCTTCTCGTTC |
| | | | CTCATCACGTAGTCGCAACAGTTCAAGAAATTCAACT |
| | | | CCAGGCAGCAGTAGGGGAACTTCTCCTGCTAGAATGG |
| | | | CTGGCAATGGCGGTGATGCTGCTCTTGCTTTGCTGCTG |
| | | | CTTGACAGATTGAACCAGCTTGAGAGCAAAATGTCTG |
| | | | GTAAAGGCCAACAACAACAAGGCCAAACTGTCACTAA |
| | | | GAAATCTGCTGCTGAGGCTTCTAAGAAGCCTCGGCAA |
| | | | AAACGTACTGCCACTAAAGCATACAATGTAACACAAG |
| | | | CTTTCGGCAGACGTGGTCCAGAACAAACCCAAGGAAA |
| | | | TTTTGGGGACCAGGAACTAATCAGACAAGGAACTGAT |
| | | | TACAAACATTGGCCGCAAATTGCACAATTTGCCCCCA |
| | | | GCGCTTCAGCGTTCTTCGGAATGTCGCGCATTGGCATG |
| | | | GAAGTCACACCTTCGGGAACGTGGTTGACCTACACAG |
| | | | GTGCCATCAAATTGGATGACAAAGATCCAAATTTCAA |
| | | | AGATCAAGTCATTTTGCTGAATAAGCATATTGACGCAT |
| | | | ACAAAACATTCCCACCAACAGAGCCTAAAAAGGACAA |
| | | | AAAGAAGAAGGCTGATGAAACTCAAGCCTTACCGCAG |
| | | | AGACAGAAGAAACAGCAAACTGTGACTCTTCTTCCTG |
| | | | CTGCAGATTTGGATGATTTCTCCAAACAATTGCAACAA |
| | | | TCCATGAGCAGTGCTGACTCAACTCAGGCCTA |

The invention covers a combination of bioinformatic approaches to identify regions of conservation (based on 718 patient isolates) in combination with features essential for RISC entry and tolerance of chemical modifications. The method uses a scoring scheme to identify siRNAs that target regions of related viral genomes with low mutation rates. Since viral targets are known to mutate frequently, it was imperative to select siRNAs targeting regions that are predicted to remain constant. We identified these regions as those with high homology to the six most closely-related coronavirus genomes (Middle East respiratory syndrome-related coronavirus (MERS-CoV), Human coronavirus 229E, Human coronavirus NL63 (HCoV-NL63), Human coronavirus HKU1 (HCoV-HKU1), Human coronavirus OC43 (HCoV-OC43), and SARS coronavirus) indicating low rates of mutation within these regions.

The percent homology to the related coronavirus genomes was determined for every position for each of the SARS-CoV-2 target genes. siRNA sequence designs were then scored by the number of positions within the sequence having a percentage homology greater than 70% within position 2-8 and greater than 50% for at least 10 bases within the remaining positions of the 16-nucleotide targeting region of the 20-nucleotide siRNA. The design algorithm identified a 20-nucleotide siRNA sequences and scored them by their predicted efficiency to knockdown the target transcript. The 20-nucleotide siRNA target regions are summarized in Table 6A and Table 7. Top scoring siRNAs had the highest potential to knockdown the target transcript and targeted regions with the highest homology to other closely related coronaviruses (Middle East respiratory syndrome-related coronavirus (MERS-CoV), Human coronavirus 229E, Human coronavirus NL63 (HCoV-NL63), Human coronavirus HKU1 (HCoV-HKU1), Human coronavirus OC43 (HCoV-OC43), and SARS coronavirus) are summarized in Table 8 and were selected for synthesis. ASOs targeting SARS-CoV-2 genes are summarized in Table 9.

TABLE 6A
SARS-CoV2 Screened-20 nucleotide target sequences and 45 nucleotide
target gene regions

| | | SEQ | | SEQ |
|---|---|---|---|---|
| | | ID | | ID |
| Sequence ID | 20 nt Sequence | NO: | 45 nt Gene Region | NO: |
| orf1ab_14080 | GGUAACUGG | 11 | AATCAAGATCTCAATGGTAACTGGTAT | 119 |
| | UAUGAUUUC | | GATTTCGGTGATTTCATA | |
| | GG | | | |
| orf1ab_14361 | UCUGCAUUG | 12 | GGATGACAGATGCATTCTGCATTGTGC | 120 |
| | UGCAAACUU | | AAACTTTAATGTTTTATT | |
| | UA | | | |
| orf1ab_14830 | UGUGAUAUC | 13 | AATCTACCAACAATGTGTGATATCAGA | 121 |
| | AGACAACUA | | CAACTACTATTTGTAGTT | |
| | CU | | | |
| orf1ab_15376 | UGUAGCUUG | 14 | AAACATACAACGTGTTGTAGCTTGTCA | 122 |
| | UCACACCGU | | CACCGTTTCTATAGATTA | |
| | UU | | | |
| orf1ab_15786 | UAAGUCAGU | 15 | TAGCATAAAGAACTTTAAGTCAGTTCT | 123 |
| | UCUUUAUUA | | TTATTATCAAAACAATGT | |
| | UC | | | |
| orf1ab_17107 | UUUGCUAUU | 16 | ACTGGTAAGAGTCATTTTGCTATTGGC | 124 |
| | GGCCUAGCU | | CTAGCTCTCTACTACCCT | |
| | CU | | | |
| orf1ab_17370 | GGCCACAAA | 17 | TGATGAAATTTCAATGGCCACAAATTA | 125 |
| | UUAUGAUU | | TGATTTGAGTGTTGTCAA | |
| | UGA | | | |
| orf1ab_18025 | GUGGCAACU | 18 | ATTCCACGTAGGAATGTGGCAACTTTA | 126 |
| | UUACAAGCU | | CAAGCTGAAAATGTAACA | |
| | GA | | | |
| orf1ab_18571 | UCUGACAGA | 19 | ACACTTAAAAATCTCTCTGACAGAGTC | 127 |
| | GUCGUAUUU | | GTATTTGTCTTATGGGCA | |
| | GU | | | |
| orf1ab_20497 | UCUGUUAUU | 20 | TCTAAGTGTGTGTGTTCTGTTATTGATT | 128 |
| | GAUUUAUU | | TATTACTTGATGATTTT | |
| | ACU | | | |
| orf1ab_20892 | UGCACCAGG | 21 | TTCTGATAAAGGAGTTGCACCAGGTAC | 129 |
| | UACAGCUGU | | AGCTGTTTTAAGACAGTG | |
| | UU | | | |
| orf1ab_21391 | UUUGACAUG | 22 | TCTTCCTATTCTTTATTTGACATGAGTA | 130 |
| | AGUAAAUU | | AATTTCCCCTTAAATTA | |
| | UCC | | | |
| orf1a_416 | UGUGGCUUA | 23 | CTTAAAGATGGCACTTGTGGCTTAGTA | 131 |
| | GUAGAAGU | | GAAGTTGAAAAAGGCGTT | |
| | UGA | | | |
| orf1a_2290 | UCAGACAUU | 24 | AATTAAGGAGAGTGTTCAGACATTCTT | 132 |
| | CUUUAAGCU | | TAAGCTTGTAAATAAATT | |
| | UG | | | |
| orf1a_6059 | UGUGAUAA | 25 | AATTTTAAGTTTGTATGTGATAATATC | 133 |
| | UAUCAAAUU | | AAATTTGCTGATGATTTA | |
| | UGC | | | |
| orf1a_6322 | UGAAACAUC | 26 | GAGCACAAAACCAGTTGAAACATCAA | 134 |
| | AAAUUCGUU | | ATTCGTTTGATGTACTGAA | |
| | UG | | | |
| orf1a_6499 | ACCAGCAAA | 27 | AGACATTATACTTAAACCAGCAAATAA | 135 |
| | UAAUAGUU | | TAGTTTAAAAATTACAGA | |
| | UAA | | | |
| orf1a_7643 | GCUGGUAGU | 28 | TGTGATACATTCTGTGCTGGTAGTACA | 136 |
| | ACAUUUAUU | | TTTATTAGTGATGAAGTT | |
| | AG | | | |
| orf1a_8200 | UGUAGAAAC | 29 | GTTTGTTGATTCAGATGTAGAAACTAA | 137 |
| | UAAAGAUG | | AGATGTTGTTGAATGTCT | |
| | UUG | | | |
| orf1a_8201 | GUAGAAACU | 30 | TTTGTTGATTCAGATGTAGAAACTAAA | 138 |
| | AAAGAUGU | | GATGTTGTTGAATGTCTT | |
| | UGU | | | |
| orf1a_8744 | UUUGCUAAC | 31 | TCTACAGATACTTGTTTTGCTAACAAA | 139 |
| | AAACAUGCU | | CATGCTGATTTTGACACA | |
| | GA | | | |
| orf1a_9679 | CUGGAUAAC | 32 | ACCTTTAGTACCTTTCTGGATAACAAT | 140 |
| | AAUUGCUUA | | TGCTTATATCATTTGTAT | |
| | UA | | | |
| orf1a_11594 | CAGUGUAUA | 33 | ACTGGTAATACACTTCAGTGTATAATG | 141 |
| | AUGCUAGUU | | CTAGTTTATTGTTTCTTA | |
| | UA | | | |
| orf1a_12932 | CCUAAAGUG | 34 | GACACACCTAAAGGTCCTAAAGTGAA | 142 |
| | AAGUAUUU | | GTATTTATACTTTATTAAA | |
| | AUA | | | |
| S_21944 | AUUAAAGUC | 35 | GCTACTAATGTTGTTATTAAAGTCTGT | 143 |
| | UGUGAAUU | | GAATTTCAATTTTGTAAT | |
| | UCA | | | |
| S_22223 | UCGGCUUUA | 36 | CTCCCTCAGGGTTTTTCGGCTTTAGAA | 144 |
| | GAACCAUUG | | CCATTGGTAGATTTGCCA | |
| | GU | | | |
| S_22550 | CCUAAUAUU | 37 | TCTATTGTTAGATTTCCTAATATTACAA | 145 |
| | ACAAACUUG | | ACTTGTGCCCTTTTGGT | |
| | UG | | | |
| S_22820 | GAUUAUAA | 38 | ACTGGAAAGATTGCTGATTATAATTAT | 146 |
| | UUAUAAAU | | AAATTACCAGATGATTTT | |
| | UACC | | | |
| S_22898 | GGUGGUAA | 39 | CTTGATTCTAAGGTTGGTGGTAATTAT | 147 |
| | UUAUAAUU | | AATTACCTGTATAGATTG | |
| | ACCU | | | |
| S_23174 | UGUGUCAAU | 40 | TTGGTTAAAAACAAATGTGTCAATTTC | 148 |
| | UUCAACUUC | | AACTTCAATGGTTTAACA | |
| | AA | | | |
| S_23239 | UCUGCCUUU | 41 | GTCTAACAAAAAGTTTCTGCCTTTCCA | 149 |
| | CCAACAAUU | | ACAATTTGGCAGAGACAT | |
| | UG | | | |
| S_23240 | CUGCCUUUC | 42 | TCTAACAAAAAGTTTCTGCCTTTCCAA | 150 |
| | CAACAAUUU | | CAATTTGGCAGAGACATT | |
| | GG | | | |
| S_23774 | UGUACAAUG | 43 | AAGACATCAGTAGATTGTACAATGTAC | 151 |
| | UACAUUUGU | | ATTTGTGGTGATTCAACT | |
| | GG | | | |
| S_24056 | GGCUUCAUC | 44 | ACACTTGCAGATGCTGGCTTCATCAAA | 152 |
| | AAACAAUAU | | CAATATGGTGATTGCCTT | |
| | GG | | | |
| S_24289 | UGGAGUUAC | 45 | TAGGTTTAATGGTATTGGAGTTACACA | 153 |
| | ACAGAAUGU | | GAATGTTCTCTATGAGAA | |
| | UC | | | |
| S_25375 | UUACACAUA | 46 | AGGAGTCAAATTACATTACACATAAAC | 154 |
| | AACGAACUU | | GAACTTATGGATTTGTTT | |
| | AU | | | |
| 3a_25413 | CUUCACAAU | 47 | TTTGTTTATGAGAATCTTCACAATTGG | 155 |
| | UGGAACUGU | | AACTGTAACTTTGAAGCA | |
| | AA | | | |
| 3a_25630 | GUUUGCAAC | 48 | AAGGGTGTTCACTTTGTTTGCAACTTG | 156 |
| | UUGCUGUUG | | CTGTTGTTGTTTGTAACA | |
| | UU | | | |
| 3a_25717 | UAUGCUUUA | 49 | CCTTTTCTCTATCTTTATGCTTTAGTCT | 157 |
| | GUCUACUUC | | ACTTCTTGCAGAGTATA | |
| | UU | | | |
| 3a_25734 | CUUGCAGAG | 50 | TGCTTTAGTCTACTTCTTGCAGAGTAT | 158 |
| | UAUAAACUU | | AAACTTTGTAAGAATAAT | |
| | UG | | | |
| 3a_25736 | UGCAGAGUA | 51 | CTTTAGTCTACTTCTTGCAGAGTATAA | 159 |
| | UAAACUUUG | | ACTTTGTAAGAATAATAA | |
| | UA | | | |
| 3a_25745 | UAAACUUUG | 52 | ACTTCTTGCAGAGTATAAACTTTGTAA | 160 |
| | UAAGAAUA | | GAATAATAATGAGGCTTT | |
| | AUA | | | |
| 3a_25868 | CUUACAAUA | 53 | ACGACTATTGTATACCTTACAATAGTG | 161 |
| | GUGUAACUU | | TAACTTCTTCAATTGTCA | |
| | CU | | | |
| 3a_25870 | UACAAUAGU | 54 | GACTATTGTATACCTTACAATAGTGTA | 162 |
| | GUAACUUCU | | ACTTCTTCAATTGTCATT | |
| | UC | | | |
| 3a_25914 | CACAACAAG | 55 | TACTTCAGGTGATGGCACAACAAGTCC | 163 |
| | UCCUAUUUC | | TATTTCTGAACATGACTA | |
| | UG | | | |
| 3a_25992 | UGUUGUAU | 56 | TGGAGTAAAAGACTGTGTTGTATTACA | 164 |
| | UACACAGUU | | CAGTTACTTCACTTCAGA | |
| | ACU | | | |
| 3a_26018 | CAGACUAUU | 57 | ACAGTTACTTCACTTCAGACTATTACC | 165 |
| | ACCAGCUGU | | AGCTGTACTCAACTCAAT | |
| | AC | | | |
| 3a_26066 | UUGAACAUG | 58 | GTACAGACACTGGTGTTGAACATGTTA | 166 |
| | UUACCUUCU | | CCTTCTTCATCTACAATA | |
| | UC | | | |
| E_26258 | UUUCGGAAG | 59 | TTATGTACTCATTCGTTTCGGAAGAGA | 167 |
| | AGACAGGUA | | CAGGTACGTTAATAGTTA | |
| | CG | | | |
| E_26261 | CGGAAGAGA | 60 | TGTACTCATTCGTTTCGGAAGAGACAG | 168 |
| | CAGGUACGU | | GTACGTTAATAGTTAATA | |
| | UA | | | |
| E_26269 | ACAGGUACG | 61 | TTCGTTTCGGAAGAGACAGGTACGTTA | 169 |
| | UUAAUAGU | | ATAGTTAATAGCGTACTT | |
| | UAA | | | |
| E_26277 | GUUAAUAG | 62 | GGAAGAGACAGGTACGTTAATAGTTA | 170 |
| | UUAAUAGCG | | ATAGCGTACTTCTTTTTCT | |
| | UAC | | | |
| E_26305 | CUUGCUUUC | 63 | AGCGTACTTCTTTTTCTTGCTTTCGTGG | 171 |
| | GUGGUAUUC | | TATTCTTGCTAGTTACA | |
| | UU | | | |
| E_26313 | CGUGGUAUU | 64 | TCTTTTTCTTGCTTTCGTGGTATTCTTG | 172 |
| | CUUGCUAGU | | CTAGTTACACTAGCCAT | |
| | UA | | | |
| E_26369 | ACUGCUGCA | 65 | TTCGATTGTGTGCGTACTGCTGCAATA | 173 |
| | AUAUUGUU | | TTGTTAACGTGAGTCTTG | |
| | AAC | | | |
| E_26374 | UGCAAUAUU | 66 | TTGTGTGCGTACTGCTGCAATATTGTT | 174 |
| | GUUAACGUG | | AACGTGAGTCTTGTAAAA | |
| | AG | | | |
| E_26455 | CCUGAUCUU | 67 | AATTCTTCTAGAGTTCCTGATCTTCTGG | 175 |
| | CUGGUCUAA | | TCTAAACGAACTAAATA | |
| | AC | | | |
| E_26463 | UCUGGUCUA | 68 | TAGAGTTCCTGATCTTCTGGTCTAAAC | 176 |
| | AACGAACUA | | GAACTAAATATTATATTA | |
| | AA | | | |
| E_26467 | GUCUAAACG | 69 | GTTCCTGATCTTCTGGTCTAAACGAAC | 177 |
| | AACUAAAUA | | TAAATATTATATTAGTTT | |
| | UU | | | |
| E_26470 | UAAACGAAC | 70 | CCTGATCTTCTGGTCTAAACGAACTAA | 178 |
| | UAAAUAUU | | ATATTATATTAGTTTTTC | |
| | AUA | | | |
| M_26573 | UGAACAAUG | 71 | GCTTAAAAAGCTCCTTGAACAATGGAA | 179 |
| | GAACCUAGU | | CCTAGTAATAGGTTTCCT | |
| | AA | | | |
| M_26581 | GGAACCUAG | 72 | AGCTCCTTGAACAATGGAACCTAGTAA | 180 |
| | UAAUAGGU | | TAGGTTTCCTATTCCTTA | |
| | UUC | | | |
| M_26602 | UAUUCCUUA | 73 | TAGTAATAGGTTTCCTATTCCTTACAT | 181 |
| | CAUGGAUUU | | GGATTTGTCTTCTACAAT | |
| | GU | | | |
| M_26624 | UCUACAAUU | 74 | TACATGGATTTGTCTTCTACAATTTGCC | 182 |
| | UGCCUAUGC | | TATGCCAACAGGAATAG | |
| | CA | | | |
| M_26637 | UAUGCCAAC | 75 | CTTCTACAATTTGCCTATGCCAACAGG | 183 |
| | AGGAAUAG | | AATAGGTTTTTGTATATA | |
| | GUU | | | |
| M_26638 | AUGCCAACA | 76 | TTCTACAATTTGCCTATGCCAACAGGA | 184 |
| | GGAAUAGG | | ATAGGTTTTTGTATATAA | |
| | UUU | | | |
| M_26693 | AUGGCCAGU | 77 | TTTCCTCTGGCTGTTATGGCCAGTAAC | 185 |
| | AACUUUAGC | | TTTAGCTTGTTTTGTGCT | |
| | UU | | | |
| M_26717 | UGUGCUUGC | 78 | AACTTTAGCTTGTTTTGTGCTTGCTGCT | 186 |
| | UGCUGUUUA | | GTTTACAGAATAAATTG | |
| | CA | | | |
| M_27014 | GCCUAAAGA | 79 | TGACATCAAGGACCTGCCTAAAGAAA | 187 |
| | AAUCACUGU | | TCACTGTTGCTACATCACG | |
| | UG | | | |
| M_27032 | UGCUACAUC | 80 | TAAAGAAATCACTGTTGCTACATCACG | 188 |
| | ACGAACGCU | | AACGCTTTCTTATTACAA | |
| | UU | | | |
| M_27035 | UACAUCACG | 81 | AGAAATCACTGTTGCTACATCACGAAC | 189 |
| | AACGCUUUC | | GCTTTCTTATTACAAATT | |
| | UU | | | |
| M_27123 | AUUGGCAAC | 82 | TACAGTCGCTACAGGATTGGCAACTAT | 190 |
| | UAUAAAUU | | AAATTAAACACAGACCAT | |
| | AAA | | | |
| 7a_27455 | AAGAGUGU | 83 | AGCTTTATCACTACCAAGAGTGTGTTA | 191 |
| | GUUAGAGG | | GAGGTACAACAGTACTTT | |
| | UACA | | | |
| 7a_27522 | UUCACCAUU | 84 | AACATACGAGGGCAATTCACCATTTCA | 192 |
| | UCAUCCUCU | | TCCTCTAGCTGATAACAA | |
| | AG | | | |
| 7a_27537 | UCUAGCUGA | 85 | TTCACCATTTCATCCTCTAGCTGATAA | 193 |
| | UAACAAAUU | | CAAATTTGCACTGACTTG | |
| | UG | | | |
| 7a_27553 | UUUGCACUG | 86 | CTAGCTGATAACAAATTTGCACTGACT | 194 |
| | ACUUGCUUU | | TGCTTTAGCACTCAATTT | |
| | AG | | | |
| 7a_27565 | UGCUUUAGC | 87 | AAATTTGCACTGACTTGCTTTAGCACT | 195 |
| | ACUCAAUUU | | CAATTTGCTTTTGCTTGT | |
| | GC | | | |
| 7a_27633 | AUCAGUUUC | 88 | TCAGTTACGTGCCAGATCAGTTTCACC | 196 |
| | ACCUAAACU | | TAAACTGTTCATCAGACA | |
| | GU | | | |
| 7a_27656 | UCAGACAAG | 89 | CACCTAAACTGTTCATCAGACAAGAGG | 197 |
| | AGGAAGUUC | | AAGTTCAAGAACTTTACT | |
| | AA | | | |
| 7a_27671 | UUCAAGAAC | 90 | TCAGACAAGAGGAAGTTCAAGAACTT | 198 |
| | UUUACUCUC | | TACTCTCCAATTTTTCTTA | |
| | CA | | | |
| 7a_27705 | UGCGGCAAU | 91 | AATTTTTCTTATTGTTGCGGCAATAGT | 199 |
| | AGUGUUUA | | GTTTATAACACTTTGCTT | |
| | UAA | | | |
| 7a_27715 | GUGUUUAU | 92 | ATTGTTGCGGCAATAGTGTTTATAACA | 200 |
| | AACACUUUG | | CTTTGCTTCACACTCAAA | |
| | CUU | | | |
| 7a_27720 | UAUAACACU | 93 | TGCGGCAATAGTGTTTATAACACTTTG | 201 |
| | UUGCUUCAC | | CTTCACACTCAAAAGAAA | |
| | AC | | | |
| 7a_27751 | ACAGAAUGA | 94 | ACACTCAAAAGAAAGACAGAATGATT | 202 |
| | UUGAACUUU | | GAACTTTCATTAATTGACT | |
| | CA | | | |
| 8b_27932 | AGCUGCAUU | 95 | AATCATCACAACTGTAGCTGCATTTCA | 203 |
| | UCACCAAGA | | CCAAGAATGTAGTTTACA | |
| | AU | | | |
| 8b_27940 | UUCACCAAG | 96 | CAACTGTAGCTGCATTTCACCAAGAAT | 204 |
| | AAUGUAGU | | GTAGTTTACAGTCATGTA | |
| | UUA | | | |
| 8b_27986 | UGUAGUUG | 97 | TCAACATCAACCATATGTAGTTGATGA | 205 |
| | AUGACCCGU | | CCCGTGTCCTATTCACTT | |
| | GUC | | | |
| 8b_28002 | UGUCCUAUU | 98 | GTAGTTGATGACCCGTGTCCTATTCAC | 206 |
| | CACUUCUAU | | TTCTATTCTAAATGGTAT | |
| | UC | | | |
| 8b_28024 | AAUGGUAU | 99 | TTCACTTCTATTCTAAATGGTATATTAG | 207 |
| | AUUAGAGU | | AGTAGGAGCTAGAAAAT | |
| | AGGA | | | |
| 8b_28091 | UUCUAAAUC | 100 | CGTGGATGAGGCTGGTTCTAAATCACC | 208 |
| | ACCCAUUCA | | CATTCAGTACATCGATAT | |
| | GU | | | |
| 8b_28119 | AUCGGUAAU | 101 | ATTCAGTACATCGATATCGGTAATTAT | 209 |
| | UAUACAGUU | | ACAGTTTCCTGTTTACCT | |
| | UC | | | |
| 8b_28127 | UUAUACAGU | 102 | CATCGATATCGGTAATTATACAGTTTC | 210 |
| | UUCCUGUUU | | CTGTTTACCTTTTACAAT | |
| | AC | | | |
| 8b_28128 | UAUACAGUU | 103 | ATCGATATCGGTAATTATACAGTTTCC | 211 |
| | UCCUGUUUA | | TGTTTACCTTTTACAATT | |
| | CC | | | |
| 8b_28163 | CCAGGAACC | 104 | TTTTACAATTAATTGCCAGGAACCTAA | 212 |
| | UAAAUUGG | | ATTGGGTAGTCTTGTAGT | |
| | GUA | | | |
| 8b_28218 | UUAGAGUA | 105 | TTCTATGAAGACTTTTTAGAGTATCAT | 213 |
| | UCAUGACGU | | GACGTTCGTGTTGTTTTA | |
| | UCG | | | |
| 8b_28222 | AGUAUCAUG | 106 | ATGAAGACTTTTTAGAGTATCATGACG | 214 |
| | ACGUUCGUG | | TTCGTGTTGTTTTAGATT | |
| | UU | | | |
| N_28407 | UACCCAAUA | 107 | GTCGGCCCCAAGGTTTACCCAATAATA | 215 |
| | AUACUGCGU | | CTGCGTCTTGGTTCACCG | |
| | CU | | | |
| N_28655 | GACGGCAUC | 108 | TATGGTGCTAACAAAGACGGCATCATA | 216 |
| | AUAUGGGU | | TGGGTTGCAACTGAGGGA | |
| | UGC | | | |
| N_28945 | UGACAGAUU | 109 | TGCTTTGCTGCTGCTTGACAGATTGAA | 217 |
| | GAACCAGCU | | CCAGCTTGAGAGCAAAAT | |
| | UG | | | |
| N_28992 | AACAACAAG | 110 | CTGGTAAAGGCCAACAACAACAAGGC | 218 |
| | GCCAAACUG | | CAAACTGTCACTAAGAAAT | |
| | UC | | | |
| N_29141 | GAACUAAUC | 111 | AATTTTGGGGACCAGGAACTAATCAG | 219 |
| | AGACAAGGA | | ACAAGGAACTGATTACAAA | |
| | AC | | | |
| N_29276 | GGUGCCAUC | 112 | TGGTTGACCTACACAGGTGCCATCAAA | 220 |
| | AAAUUGGA | | TTGGATGACAAAGATCCA | |
| | UGA | | | |
| N_29292 | AUGACAAAG | 113 | GTGCCATCAAATTGGATGACAAAGATC | 221 |
| | AUCCAAAUU | | CAAATTTCAAAGATCAAG | |
| | UC | | | |
| N_29293 | UGACAAAGA | 114 | TGCCATCAAATTGGATGACAAAGATCC | 222 |
| | UCCAAAUUU | | AAATTTCAAAGATCAAGT | |
| | CA | | | |
| N_29303 | CCAAAUUUC | 115 | TTGGATGACAAAGATCCAAATTTCAAA | 223 |
| | AAAGAUCAA | | GATCAAGTCATTTTGCTG | |
| | GU | | | |
| N_29307 | AUUUCAAAG | 116 | ATGACAAAGATCCAAATTTCAAAGATC | 224 |
| | AUCAAGUCA | | AAGTCATTTTGCTGAATA | |
| | UU | | | |
| N_29328 | UGCUGAAUA | 117 | AAGATCAAGTCATTTTGCTGAATAAGC | 225 |
| | AGCAUAUUG | | ATATTGACGCATACAAAA | |
| | AC | | | |
| N_29464 | UGCAGAUUU | 118 | GACTCTTCTTCCTGCTGCAGATTTGGA | 226 |
| | GGAUGAUU | | TGATTTCTCCAAACAATT | |
| | UCU | | | |

TABLE 6B
Modified antisense strand (21 nucleotide length for screening)

| | | SEQ |
|---|---|---|
| | | ID |
| Sequence ID | Modified AS strand (21mer for screening) | NO: |
| orf1ab_14080 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_14361 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_14830 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_15376 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_15786 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_17107 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_17370 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_18025 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_18571 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_20497 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_20892 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1ab_21391 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_416 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_2290 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_6059 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_6322 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_6499 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_7643 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_8200 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_8201 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_8744 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_9679 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_11594 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| orf1a_12932 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_21944 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_22223 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_22550 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_22820 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_22898 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_23174 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_23239 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_23240 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_23774 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_24056 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_24289 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| S_25375 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_25413 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_25630 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_25717 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_25734 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_25736 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_25745 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_25868 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_25870 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_25914 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_25992 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_26018 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 3a_26066 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26258 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26261 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26269 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26277 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26305 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26313 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26369 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26374 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26455 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26463 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26467 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| E_26470 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_26573 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_26581 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_26602 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_26624 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_26637 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_26638 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_26693 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_26717 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_27014 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_27032 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_27035 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| M_27123 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27455 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27522 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27537 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27553 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a 27565 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27633 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27656 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27671 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27705 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27715 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27720 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 7a_27751 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_27932 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_27940 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_27986 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_28002 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_28024 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_28091 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_28119 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_28127 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_28128 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_28163 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_28218 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| 8b_28222 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_28407 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_28655 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_28945 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_28992 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_29141 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_29276 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_29292 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_29293 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_29303 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_29307 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_29328 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |
| N_29464 | P(mU)#(fA)#(mA)(mC)(mA)(fU)(mG)(mA)(mA)(mU)(mA)(mC)(mU)(fU)#(mG)#(fG)#(mC)#(mU)#(mU)#(mU)#(mU) | 227 |

TABLE 6C
Modified sense strand-16 nucleotides in length

| | | SEQ |
|---|---|---|
| Sequence | | ID |
| ID | Modified sense strand 16 nt | NO: |
| orf1ab_14080 | (mA)#(mU)#(mC)(mA)(fU)(fA)(fC)(mC)(fA)(mG)(mU)(mU)(mA)#(mC)#(mA)-TegChol | 228 |
| orf1ab_14361 | (mG)#(mU)#(mU)(mU)(mG)(fC)(fA)(fC)(mA)(fA)(mU)(mG)(mC)(mA)#(mG)#(mA)-TegChol | 229 |
| orf1ab_14830 | (mU)#(mU)#(mU)(mG)(fC)(fA)(fC)(mA)(fA)(mU)(mG)(mC)(mA)#(mG)#(mA)-TegChol | 230 |
| orf1ab_15376 | (mG)#(mU)#(mG)(mU)(fG)(fA)(fC)(mA)(fA)(mG)(mC)(mU)(mA)#(mC)#(mA)-TegChol | 231 |
| orf1ab_15786 | (mU)#(mA)#(mA)(mA)(fG)(fA)(fA)(mC)(fU)(mG)(mA)(mC)(mU)#(mU)#(mA)-TegChol | 232 |
| orf1ab_17107 | (mU)#(mA)#(mG)(mG)(fC)(fC)(fA)(mA)(fU)(mA)(mG)(mC)(mA)#(mA)#(mA)-TegChol | 233 |
| orf1ab_17370 | (mU)#(mC)#(mA)(mU)(fA)(fA)(fU)(mU)(fU)(mG)(mU)(mG)(mG)#(mC)#(mA)-TegChol | 234 |
| orf1ab_18025 | (mU)#(mU)#(mG)(mU)(fA)(fA)(fA)(mG)(fU)(mU)(mG)(mC)(mC)#(mA)#(mA)-TegChol | 235 |
| orf1ab_18571 | (mU)#(mA)#(mC)(mG)(fA)(fC)(fU)(mC)(fU)(mG)(mU)(mC)(mA)#(mG)#(mA)-TegChol | 236 |
| orf1ab_20497 | (mU)#(mA)#(mA)(mA)(fU)(fC)(fA)(mA)(fU)(mA)(mA)(mC)(mA)#(mG)#(mA)-TegChol | 237 |
| orf1ab_20892 | (mG)#(mC)#(mU)(mG)(fU)(fA)(fC)(mC)(fU)(mG)(mG)(mU)(mG)#(mC)#(mA)-TegChol | 238 |
| orf1ab_21391 | (mU)#(mU)#(mU)(mA)(fC)(fU)(fC)(mA)(fU)(mG)(mU)(mC)(mA)#(mA)#(mA)-TegChol | 239 |
| orf1a_416 | (mU)#(mU)#(mC)(mU)(fA)(fC)(fU)(mA)(fA)(mG)(mC)(mC)(mA)#(mC)#(mA)-TegChol | 240 |
| orf1a_2290 | (mU)#(mU)#(mA)(mA)(fA)(fG)(fA)(mA)(fU)(mG)(mU)(mC)(mU)#(mG)#(mA)-TegChol | 241 |
| orf1a_6059 | (mU)#(mU)#(mU)(mG)(fA)(fU)(fA)(mU)(fU)(mA)(mU)(mC)(mA)#(mC)#(mA)-TegChol | 242 |
| orf1a_6322 | (mG)#(mA)#(mA)(mU)(fU)(fU)(fG)(mA)(fU)(mG)(mU)(mU)(mU)#(mC)#(mA)-TegChol | 243 |
| orf1a_6499 | (mC)#(mU)#(mA)(mU)(fU)(fA)(fU)(mU)(fU)(mG)(mC)(mU)(mG)#(mG)#(mA)-TegChol | 244 |
| orf1a_7643 | (mA)#(mA)#(mA)(mU)(fG)(fU)(fA)(mC)(fU)(mA)(mC)(mC)(mA)#(mG)#(mA)-TegChol | 245 |
| orf1a_8200 | (mU)#(mC)#(mU)(mU)(fU)(fA)(fG)(mU)(fU)(mU)(mC)(mU)(mA)#(mC)#(mA)-TegChol | 246 |
| orf1a_8201 | (mA)#(mU)#(mC)(mU)(fU)(fU)(fA)(mG)(fU)(mU)(mU)(mC)(mU)#(mA)#(mA)-TegChol | 247 |
| orf1a_8744 | (mA)#(mU)#(mG)(mU)(fU)(fU)(fG)(mU)(fU)(mA)(mG)(mC)(mA)#(mA)#(mA)-TegChol | 248 |
| orf1a_9679 | (mG)#(mC)#(mA)(mA)(fU)(fU)(fG)(mU)(fU)(mA)(mU)(mC)(mC)#(mA)#(mA)-TegChol | 249 |
| orf1a_11594 | (mU)#(mA)#(mG)(mC)(fA)(fU)(fU)(mA)(fU)(mA)(mC)(mA)(mC)#(mU)#(mA)-TegChol | 250 |
| orf1a_12932 | (mA)#(mU)#(mA)(mC)(fU)(fU)(fC)(mA)(fC)(mU)(mU)(mU)(mA)#(mG)#(mA)-TegChol | 251 |
| S_21944 | (mU)#(mU)#(mC)(mA)(fC)(fA)(fG)(mA)(fC)(mU)(mU)(mU)(mA)#(mA)#(mA)-TegChol | 252 |
| S_22223 | (mU)#(mG)#(mG)(mU)(fU)(fC)(fU)(mA)(fA)(mA)(mG)(mC)(mC)#(mG)#(mA)-TegChol | 253 |
| S_22550 | (mG)#(mU)#(mU)(mU)(fG)(fU)(fA)(mA)(fU)(mA)(mU)(mU)(mA)#(mG)#(mA)-TegChol | 254 |
| S_22820 | (mU)#(mU)#(mU)(mA)(fU)(fA)(fA)(mU)(fU)(mA)(mU)(mA)(mA)#(mU)#(mA)-TegChol | 255 |
| S_22898 | (mA)#(mU)#(mU)(mA)(fU)(fA)(fA)(mU)(fU)(mA)(mC)(mC)(mA)#(mC)#(mA)-TegChol | 256 |
| S_23174 | (mG)#(mU)#(mU)(mG)(fA)(fA)(fA)(mU)(fU)(mG)(mA)(mC)(mA)#(mC)#(mA)-TegChol | 257 |
| S_23239 | (mU)#(mG)#(mU)(mU)(fG)(fG)(fA)(mA)(fA)(mG)(mG)(mC)(mA)#(mG)#(mA)-TegChol | 258 |
| S_23240 | (mU)#(mU)#(mG)(mU)(fU)(fG)(fG)(mA)(fA)(mA)(mG)(mG)(mC)#(mA)#(mA)-TegChol | 259 |
| S_23774 | (mA)#(mA)#(mU)(mG)(fU)(fA)(fC)(mA)(fU)(mU)(mG)(mU)(mA)#(mC)#(mA)-TegChol | 260 |
| S_24056 | (mU)#(mU)#(mG)(mU)(fU)(fU)(fG)(mA)(fU)(mG)(mA)(mA)(mG)#(mC)#(mA)-TegChol | 261 |
| S_24289 | (mU)#(mU)#(mC)(mU)(fG)(fU)(fG)(mU)(fA)(mA)(mC)(mU)(mC)#(mC)#(mA)-TegChol | 262 |
| S_25375 | (mU)#(mU)#(mC)(mG)(fU)(fU)(fU)(mA)(fU)(mG)(mU)(mG)(mU)#(mA)#(mA)-TegChol | 263 |
| 3a_25413 | (mG)#(mU)#(mU)(mC)(fC)(fA)(fA)(mU)(fU)(mG)(mU)(mG)(mA)#(mA)#(mA)-TegChol | 264 |
| 3a_25630 | (mC)#(mA)#(mG)(mC)(fA)(fA)(fG)(mU)(fU)(mG)(mC)(mA)(mA)#(mA)#(mA)-TegChol | 265 |
| 3a_25717 | (mG)#(mU)#(mA)(mG)(fA)(fC)(fU)(mA)(fA)(mA)(mG)(mC)(mA)#(mU)#(mA)-TegChol | 266 |
| 3a_25734 | (mU)#(mU)#(mU)(mA)(fU)(fA)(fC)(mU)(fC)(mU)(mG)(mC)(mA)#(mA)#(mA)-TegChol | 267 |
| 3a_25736 | (mA)#(mG)#(mU)(mU)(fU)(fA)(fU)(mA)(fC)(mU)(mC)(mU)(mG)#(mC)#(mA)-TegChol | 268 |
| 3a_25745 | (mU)#(mU)#(mC)(mU)(fU)(fA)(fC)(mA)(fA)(mA)(mG)(mU)(mU)#(mU)#(mA)-TegChol | 269 |
| 3a_25868 | (mU)#(mU)#(mA)(mC)(fA)(fC)(fU)(mA)(fU)(mU)(mG)(mU)(mA)#(mA)#(mA)-TegChol | 270 |
| 3a_25870 | (mA)#(mG)#(mU)(mU)(fA)(fC)(fA)(mC)(fU)(mA)(mU)(mU)(mG)#(mU)#(mA)-TegChol | 271 |
| 3a_25914 | (mA)#(mU)#(mA)(mG)(fG)(fA)(fC)(mU)(fU)(mG)(mU)(mU)(mG)#(mU)#(mA)-TegChol | 272 |
| 3a_25992 | (mC)#(mU)#(mG)(mU)(fG)(fU)(fA)(mA)(fU)(mA)(mC)(mA)(mA)#(mC)#(mA)-TegChol | 273 |
| 3a_26018 | (mG)#(mC)#(mU)(mG)(fG)(fU)(fA)(mA)(fU)(mA)(mG)(mU)(mC)#(mU)#(mA)-TegChol | 274 |
| 3a_26066 | (mA)#(mG)#(mG)(mU)(fA)(fA)(fC)(mA)(fU)(mG)(mU)(mU)(mC)#(mA)#(mA)-TegChol | 275 |
| E_26258 | (mC)#(mU)#(mG)(mU)(fC)(fU)(fC)(mU)(fU)(mC)(mC)(mG)(mA)#(mA)#(mA)-TegChol | 276 |
| E_26261 | (mU)#(mA)#(mC)(mC)(fU)(fG)(fU)(mC)(fU)(mC)(mU)(mU)(mC)#(mC)#(mA)-TegChol | 277 |
| E_26269 | (mU)#(mA)#(mU)(mU)(fA)(fA)(fC)(mG)(fU)(mA)(mC)(mC)(mU)#(mG)#(mA)-TegChol | 278 |
| E_26277 | (mC)#(mU)#(mA)(mU)(fU)(fA)(fA)(mC)(fU)(mA)(mU)(mU)(mA)#(mA)#(mA)-TegChol | 279 |
| E_26305 | (mU)#(mA)#(mC)(mC)(fA)(fC)(fG)(mA)(fA)(mA)(mG)(mC)(mA)#(mA)#(mA)-TegChol | 280 |
| E_26313 | (mA)#(mG)#(mC)(mA)(fA)(fG)(fA)(mA)(fU)(mA)(mC)(mC)(mA)#(mC)#(mA)-TegChol | 281 |
| E_26369 | (mC)#(mA)#(mA)(mU)(fA)(fU)(fU)(mG)(fC)(mA)(mG)(mC)(mA)#(mG)#(mA)-TegChol | 282 |
| E_26374 | (mG)#(mU)#(mU)(mA)(fA)(fC)(fA)(mA)(fU)(mA)(mU)(mU)(mG)#(mC)#(mA)-TegChol | 283 |
| E_26455 | (mG)#(mA)#(mC)(mC)(fA)(fG)(fA)(mA)(fG)(mA)(mU)(mC)(mA)#(mG)#(mA)-TegChol | 284 |
| E_26463 | (mU)#(mU)#(mC)(mG)(fU)(fU)(fU)(mA)(fG)(mA)(mC)(mC)(mA)#(mG)#(mA)-TegChol | 285 |
| E_26467 | (mU)#(mU)#(mA)(mG)(fU)(fU)(fC)(mG)(fU)(mU)(mU)(mA)(mG)#(mA)#(mA)-TegChol | 286 |
| E_26470 | (mU)#(mA)#(mU)(mU)(fU)(fA)(fG)(mU)(fU)(mC)(mG)(mU)(mU)#(mU)#(mA)-TegChol | 287 |
| M_26573 | (mA)#(mG)#(mG)(mU)(fU)(fC)(fC)(mA)(fU)(mU)(mG)(mU)(mU)#(mC)#(mA)-TegChol | 288 |
| M_26581 | (mC)#(mU)#(mA)(mU)(fU)(fA)(fC)(mU)(fA)(mG)(mG)(mU)(mU)#(mC)#(mA)-TegChol | 289 |
| M_26602 | (mU)#(mC)#(mC)(mA)(fU)(fG)(fU)(mA)(fA)(mG)(mG)(mA)(mA)#(mU)#(mA)-TegChol | 290 |
| M_26624 | (mU)#(mA)#(mG)(mG)(fC)(fA)(fA)(mA)(fU)(mU)(mG)(mU)(mA)#(mG)#(mA)-TegChol | 291 |
| M_26637 | (mA)#(mU)#(mU)(mC)(fC)(fU)(fG)(mU)(fU)(mG)(mG)(mC)(mA)#(mU)#(mA)-TegChol | 292 |
| M_26638 | (mU)#(mA)#(mU)(mU)(fC)(fC)(fU)(mG)(fU)(mU)(mG)(mG)(mC)#(mA)#(mA)-TegChol | 293 |
| M_26693 | (mA)#(mA)#(mA)(mG)(fU)(fU)(fA)(mC)(fU)(mG)(mG)(mC)(mC)#(mA)#(mA)-TegChol | 294 |
| M_26717 | (mA)#(mC)#(mA)(mG)(fC)(fA)(fG)(mC)(fA)(mA)(mG)(mC)(mA)#(mC)#(mA)-TegChol | 295 |
| M_27014 | (mG)#(mU)#(mG)(mA)(fU)(fU)(fU)(mC)(fU)(mU)(mU)(mA)(mG)#(mG)#(mA)-TegChol | 296 |
| M_27032 | (mG)#(mU)#(mU)(mC)(fG)(fU)(fG)(mA)(fU)(mG)(mU)(mA)(mG)#(mC)#(mA)-TegChol | 297 |
| M_27035 | (mA)#(mG)#(mC)(mG)(fU)(fU)(fC)(mG)(fU)(mG)(mA)(mU)(mG)#(mU)#(mA)-TegChol | 298 |
| M_27123 | (mU)#(mU)#(mU)(mA)(fU)(fA)(fG)(mU)(fU)(mG)(mC)(mC)(mA)#(mA)#(mA)-TegChol | 299 |
| 7a_27455 | (mC)#(mU)#(mC)(mU)(fA)(fA)(fC)(mA)(fC)(mA)(mC)(mU)(mC)#(mU)#(mA)-TegChol | 300 |
| 7a_27522 | (mG)#(mG)#(mA)(mU)(fG)(fA)(fA)(mA)(fU)(mG)(mG)(mU)(mG)#(mA)#(mA)-TegChol | 301 |
| 7a_27537 | (mU)#(mU)#(mG)(mU)(fU)(fA)(fU)(mC)(fA)(mG)(mC)(mU)(mA)#(mG)#(mA)-TegChol | 302 |
| 7a_27553 | (mG)#(mC)#(mA)(mA)(fG)(fU)(fC)(mA)(fG)(mU)(mG)(mC)(mA)#(mA)#(mA)-TegChol | 303 |
| 7a_27565 | (mU)#(mU)#(mG)(mA)(fG)(fU)(fG)(mC)(fU)(mA)(mA)(mA)(mG)#(mC)#(mA)-TegChol | 304 |
| 7a_27633 | (mU)#(mU)#(mA)(mG)(fG)(fU)(fG)(mA)(fA)(mA)(mC)(mU)(mG)#(mA)#(mA)-TegChol | 305 |
| 7a_27656 | (mC)#(mU)#(mU)(mC)(fC)(fU)(fC)(mU)(fU)(mG)(mU)(mC)(mU)#(mG)#(mA)-TegChol | 306 |
| 7a_27671 | (mA)#(mG)#(mU)(mA)(fA)(fA)(fG)(mU)(fU)(mC)(mU)(mU)(mG)#(mA)#(mA)-TegChol | 307 |
| 7a_27705 | (mA)#(mA)#(mC)(mA)(fC)(fU)(fA)(mU)(fU)(mG)(mC)(mC)(mG)#(mC)#(mA)-TegChol | 308 |
| 7a_27715 | (mA)#(mA)#(mG)(mU)(fG)(fU)(fU)(mA)(fU)(mA)(mA)(mA)(mC)#(mA)#(mA)-TegChol | 309 |
| 7a_27720 | (mA)#(mA)#(mG)(mC)(fA)(fA)(fA)(mG)(fU)(mG)(mU)(mU)(mA)#(mU)#(mA)-TegChol | 310 |
| 7a_27751 | (mG)#(mU)#(mU)(mC)(fA)(fA)(fU)(mC)(fA)(mU)(mU)(mC)(mU)#(mG)#(mA)-TegChol | 311 |
| 8b_27932 | (mU)#(mG)#(mG)(mU)(fG)(fA)(fA)(mA)(fU)(mG)(mC)(mA)(mG)#(mC)#(mA)-TegChol | 312 |
| 8b_27940 | (mU)#(mA)#(mC)(mA)(fU)(fU)(fC)(mU)(fU)(mG)(mG)(mU)(mG)#(mA)#(mA)-TegChol | 313 |
| 8b_27986 | (mG)#(mG)#(mG)(mU)(fC)(fA)(fU)(mC)(fA)(mA)(mC)(mU)(mA)#(mC)#(mA)-TegChol | 314 |
| 8b_28002 | (mG)#(mA)#(mA)(mG)(fU)(fG)(fA)(mA)(fU)(mA)(mG)(mG)(mA)#(mC)#(mA)-TegChol | 315 |
| 8b_28024 | (mC)#(mU)#(mC)(mU)(fA)(fA)(fU)(mA)(fU)(mA)(mC)(mC)(mA)#(mU)#(mA)-TegChol | 316 |
| 8b_28091 | (mA)#(mU)#(mG)(mG)(fG)(fU)(fG)(mA)(fU)(mU)(mU)(mA)(mG)#(mA)#(mA)-TegChol | 317 |
| 8b_28119 | (mU)#(mG)#(mU)(mA)(fU)(fA)(fA)(mU)(fU)(mA)(mC)(mC)(mG)#(mA)#(mA)-TegChol | 318 |
| 8b_28127 | (mC)#(mA)#(mG)(mG)(fA)(fA)(fA)(mC)(fU)(mG)(mU)(mA)(mU)#(mA)#(mA)-TegChol | 319 |
| 8b_28128 | (mA)#(mC)#(mA)(mG)(fG)(fA)(fA)(mA)(fC)(mU)(mG)(mU)(mA)#(mU)#(mA)-TegChol | 320 |
| 8b_28163 | (mA)#(mA)#(mU)(mU)(fU)(fA)(fG)(mG)(fU)(mU)(mC)(mC)(mU)#(mG)#(mA)-TegChol | 321 |
| 8b_28218 | (mG)#(mU)#(mC)(mA)(fU)(fG)(fA)(mU)(fA)(mC)(mU)(mC)(mU)#(mA)#(mA)-TegChol | 322 |
| 8b_28222 | (mG)#(mA)#(mA)(mC)(fG)(fU)(fC)(mA)(fU)(mG)(mA)(mU)(mA)#(mC)#(mA)-TegChol | 323 |
| N_28407 | (mC)#(mA)#(mG)(mU)(fA)(fU)(fU)(mA)(fU)(mU)(mG)(mG)(mG)#(mU)#(mA)-TegChol | 324 |
| N_28655 | (mC)#(mC)#(mA)(mU)(fA)(fU)(fG)(mA)(fU)(mG)(mC)(mC)(mG)#(mU)#(mA)-TegChol | 325 |
| N_28945 | (mU)#(mG)#(mG)(mU)(fU)(fC)(fA)(mA)(fU)(mC)(mU)(mG)(mU)#(mC)#(mA)-TegChol | 326 |
| N_28992 | (mU)#(mU)#(mU)(mG)(fG)(fC)(fC)(mU)(fU)(mG)(mU)(mU)(mG)#(mU)#(mA)-TegChol | 327 |
| N_29141 | (mU)#(mU)#(mG)(mU)(fC)(fU)(fG)(mA)(fU)(mU)(mA)(mG)(mU)#(mU)#(mA)-TegChol | 328 |
| N_29276 | (mC)#(mA)#(mA)(mU)(fU)(fU)(fG)(mA)(fU)(mG)(mG)(mC)(mA)#(mC)#(mA)-TegChol | 329 |
| N_29292 | (mU)#(mU)#(mG)(mG)(fA)(fU)(fC)(mU)(fU)(mU)(mG)(mU)(mC)#(mA)#(mA)-TegChol | 330 |
| N_29293 | (mU)#(mU)#(mU)(mG)(fG)(fA)(fU)(mC)(fU)(mU)(mU)(mG)(mU)#(mC)#(mA)-TegChol | 331 |
| N_29303 | (mA)#(mU)#(mC)(mU)(fU)(fU)(fG)(mA)(fA)(mA)(mU)(mU)(mU)#(mG)#(mA)-TegChol | 332 |
| N_29307 | (mC)#(mU)#(mU)(mG)(fA)(fU)(fC)(mU)(fU)(mU)(mG)(mA)(mA)#(mA)#(mA)-TegChol | 333 |
| N_29328 | (mU)#(mA)#(mU)(mG)(fC)(fU)(fU)(mA)(fU)(mU)(mC)(mA)(mG)#(mC)#(mA)-TegChol | 334 |
| N_29464 | (mU)#(mC)#(mA)(mU)(fC)(fC)(fA)(mA)(fA)(mU)(mC)(mU)(mG)#(mC)#(mA)-TegChol | 335 |

TABLE 6D
Modified sense strand-18 nucleotides in length

| | | SEQ |
|---|---|---|
| | | ID |
| Sequence ID | Modified sense strand 18 nt | NO: |
| orf1ab_14080 | (mG)#(mA)#(mA)(mA)(mU)(mC)(mA)(mU)(mA)(mC)(mC)(mA)(mG)(mU)(mU)(mA)#(mC)#(mA) | 336 |
| orf1ab_14361 | (mA)#(mA)#(mG)(mU)(mU)(mU)(mG)(mC)(mA)(mC)(mA)(mA)(mU)(mG)(mC)(mA)#(mG)#(mA) | 337 |
| orf1ab_14830 | (mU)#(mA)#(mG)(mU)(mU)(mG)(mU)(mC)(mU)(mG)(mA)(mU)(mA)(mU)(mC)(mA)#(mC)#(mA) | 338 |
| orf1ab_15376 | (mA)#(mC)#(mG)(mG)(mU)(mG)(mU)(mG)(mA)(mC)(mA)(mA)(mG)(mC)(mU)(mA)#(mC)#(mA) | 339 |
| orf1ab_15786 | (mU)#(mA)#(mA)(mU)(mA)(mA)(mA)(mG)(mA)(mA)(mC)(mU)(mG)(mA)(mC)(mU)#(mU)#(mA) | 340 |
| orf1ab_17107 | (mA)#(mG)#(mC)(mU)(mA)(mG)(mG)(mC)(mC)(mA)(mA)(mU)(mA)(mG)(mC)(mA)#(mA)#(mA) | 341 |
| orf1ab_17370 | (mA)#(mA)#(mA)(mU)(mC)(mA)(mU)(mA)(mA)(mU)(mU)(mU)(mG)(mU)(mG)(mG)#(mC)#(mA) | 342 |
| orf1ab_18025 | (mA)#(mG)#(mC)(mU)(mU)(mG)(mU)(mA)(mA)(mA)(mG)(mU)(mU)(mG)(mC)(mC)#(mA)#(mA) | 343 |
| orf1ab_18571 | (mA)#(mA)#(mA)(mU)(mA)(mC)(mG)(mA)(mC)(mU)(mC)(mU)(mG)(mU)(mC)(mA)#(mG)#(mA) | 344 |
| orf1ab_20497 | (mU)#(mA)#(mA)(mU)(mA)(mA)(mA)(mU)(mC)(mA)(mA)(mU)(mA)(mA)(mC)(mA)#(mG)#(mA) | 345 |
| orf1ab_20892 | (mA)#(mC)#(mA)(mG)(mC)(mU)(mG)(mU)(mA)(mC)(mC)(mU)(mG)(mG)(mU)(mG)#(mC)#(mA) | 346 |
| orf1ab_21391 | (mA)#(mA)#(mA)(mU)(mU)(mU)(mA)(mC)(mU)(mC)(mA)(mU)(mG)(mU)(mC)(mA)#(mA)#(mA) | 347 |
| orf1a_416 | (mA)#(mA)#(mC)(mU)(mU)(mC)(mU)(mA)(mC)(mU)(mA)(mA)(mG)(mC)(mC)(mA)#(mC)#(mA) | 348 |
| orf1a_2290 | (mA)#(mG)#(mC)(mU)(mU)(mA)(mA)(mA)(mG)(mA)(mA)(mU)(mG)(mU)(mC)(mU)#(mG)#(mA) | 349 |
| orf1a_6059 | (mA)#(mA)#(mA)(mU)(mU)(mU)(mG)(mA)(mU)(mA)(mU)(mU)(mA)(mU)(mC)(mA)#(mC)#(mA) | 350 |
| orf1a_6322 | (mA)#(mA)#(mC)(mG)(mA)(mA)(mU)(mU)(mU)(mG)(mA)(mU)(mG)(mU)(mU)(mU)#(mC)#(mA) | 351 |
| orf1a_6499 | (mA)#(mA)#(mA)(mC)(mU)(mA)(mU)(mU)(mA)(mU)(mU)(mU)(mG)(mC)(mU)(mG)#(mG)#(mA) | 352 |
| orf1a_7643 | (mA)#(mA)#(mU)(mA)(mA)(mA)(mU)(mG)(mU)(mA)(mC)(mU)(mA)(mC)(mC)(mA)#(mG)#(mA) | 353 |
| orf1a_8200 | (mA)#(mC)#(mA)(mU)(mC)(mU)(mU)(mU)(mA)(mG)(mU)(mU)(mU)(mC)(mU)(mA)#(mC)#(mA) | 354 |
| orf1a_8201 | (mA)#(mA)#(mC)(mA)(mU)(mC)(mU)(mU)(mU)(mA)(mG)(mU)(mU)(mU)(mC)(mU)#(mA)#(mA) | 355 |
| orf1a_8744 | (mA)#(mG)#(mC)(mA)(mU)(mG)(mU)(mU)(mU)(mG)(mU)(mU)(mA)(mG)(mC)(mA)#(mA)#(mA) | 356 |
| orf1a_9679 | (mU)#(mA)#(mA)(mG)(mC)(mA)(mA)(mU)(mU)(mG)(mU)(mU)(mA)(mU)(mC)(mC)#(mA)#(mA) | 357 |
| orf1a_11594 | (mA)#(mA)#(mC)(mU)(mA)(mG)(mC)(mA)(mU)(mU)(mA)(mU)(mA)(mC)(mA)(mC)#(mU)#(mA) | 358 |
| orf1a_12932 | (mU)#(mA)#(mA)(mA)(mU)(mA)(mC)(mU)(mU)(mC)(mA)(mC)(mU)(mU)(mU)(mA)#(mG)#(mA) | 359 |
| S_21944 | (mA)#(mA)#(mA)(mU)(mU)(mC)(mA)(mC)(mA)(mG)(mA)(mC)(mU)(mU)(mU)(mA)#(mA)#(mA) | 360 |
| S_22223 | (mC)#(mA)#(mA)(mU)(mG)(mG)(mU)(mU)(mC)(mU)(mA)(mA)(mA)(mG)(mC)(mC)#(mG)#(mA) | 361 |
| S_22550 | (mC)#(mA)#(mA)(mG)(mU)(mU)(mU)(mG)(mU)(mA)(mA)(mU)(mA)(mU)(mU)(mA)#(mG)#(mA) | 362 |
| S_22820 | (mU)#(mA)#(mA)(mU)(mU)(mU)(mA)(mU)(mA)(mA)(mU)(mU)(mA)(mU)(mA)(mA)#(mU)#(mA) | 363 |
| S_22898 | (mG)#(mU)#(mA)(mA)(mU)(mU)(mA)(mU)(mA)(mA)(mU)(mU)(mA)(mC)(mC)(mA)#(mC)#(mA) | 364 |
| S_23174 | (mG)#(mA)#(mA)(mG)(mU)(mU)(mG)(mA)(mA)(mA)(mU)(mU)(mG)(mA)(mC)(mA)#(mC)#(mA) | 365 |
| S_23239 | (mA)#(mA)#(mU)(mU)(mG)(mU)(mU)(mG)(mG)(mA)(mA)(mA)(mG)(mG)(mC)(mA)#(mG)#(mA) | 366 |
| S_23240 | (mA)#(mA)#(mA)(mU)(mU)(mG)(mU)(mU)(mG)(mG)(mA)(mA)(mA)(mG)(mG)(mC)#(mA)#(mA) | 367 |
| S_23774 | (mA)#(mC)#(mA)(mA)(mA)(mU)(mG)(mU)(mA)(mC)(mA)(mU)(mU)(mG)(mU)(mA)#(mC)#(mA) | 368 |
| S_24056 | (mA)#(mU)#(mA)(mU)(mU)(mG)(mU)(mU)(mU)(mG)(mA)(mU)(mG)(mA)(mA)(mG)#(mC)#(mA) | 369 |
| S_24289 | (mA)#(mC)#(mA)(mU)(mU)(mC)(mU)(mG)(mU)(mG)(mU)(mA)(mA)(mC)(mU)(mC)#(mC)#(mA) | 370 |
| S_25375 | (mA)#(mA)#(mG)(mU)(mU)(mC)(mG)(mU)(mU)(mU)(mA)(mU)(mG)(mU)(mG)(mU)#(mA)#(mA) | 371 |
| 3a_25413 | (mA)#(mC)#(mA)(mG)(mU)(mU)(mC)(mC)(mA)(mA)(mU)(mU)(mG)(mU)(mG)(mA)#(mA)#(mA) | 372 |
| 3a_25630 | (mC)#(mA)#(mA)(mC)(mA)(mG)(mC)(mA)(mA)(mG)(mU)(mU)(mG)(mC)(mA)(mA)#(mA)#(mA) | 373 |
| 3a_25717 | (mG)#(mA)#(mA)(mG)(mU)(mA)(mG)(mA)(mC)(mU)(mA)(mA)(mA)(mG)(mC)(mA)#(mU)#(mA) | 374 |
| 3a_25734 | (mA)#(mA)#(mG)(mU)(mU)(mU)(mA)(mU)(mA)(mC)(mU)(mC)(mU)(mG)(mC)(mA)#(mA)#(mA) | 375 |
| 3a_25736 | (mC)#(mA)#(mA)(mA)(mG)(mU)(mU)(mU)(mA)(mU)(mA)(mC)(mU)(mC)(mU)(mG)#(mC)#(mA) | 376 |
| 3a_25745 | (mU)#(mU)#(mA)(mU)(mU)(mC)(mU)(mU)(mA)(mC)(mA)(mA)(mA)(mG)(mU)(mU)#(mU)#(mA) | 377 |
| 3a_25868 | (mA)#(mA)#(mG)(mU)(mU)(mA)(mC)(mA)(mC)(mU)(mA)(mU)(mU)(mG)(mU)(mA)#(mA)#(mA) | 378 |
| 3a_25870 | (mA)#(mG)#(mA)(mA)(mG)(mU)(mU)(mA)(mC)(mA)(mC)(mU)(mA)(mU)(mU)(mG)#(mU)#(mA) | 379 |
| 3a_25914 | (mG)#(mA)#(mA)(mA)(mU)(mA)(mG)(mG)(mA)(mC)(mU)(mU)(mG)(mU)(mU)(mG)#(mU)#(mA) | 380 |
| 3a_25992 | (mU)#(mA)#(mA)(mC)(mU)(mG)(mU)(mG)(mU)(mA)(mA)(mU)(mA)(mC)(mA)(mA)#(mC)#(mA) | 381 |
| 3a_26018 | (mA)#(mC)#(mA)(mG)(mC)(mU)(mG)(mG)(mU)(mA)(mA)(mU)(mA)(mG)(mU)(mC)#(mU)#(mA) | 382 |
| 3a_26066 | (mA)#(mG)#(mA)(mA)(mG)(mG)(mU)(mA)(mA)(mC)(mA)(mU)(mG)(mU)(mU)(mC)#(mA)#(mA) | 383 |
| E_26258 | (mU)#(mA)#(mC)(mC)(mU)(mG)(mU)(mC)(mU)(mC)(mU)(mU)(mC)(mC)(mG)(mA)#(mA)#(mA) | 384 |
| E_26261 | (mA)#(mC)#(mG)(mU)(mA)(mC)(mC)(mU)(mG)(mU)(mC)(mU)(mC)(mU)(mU)(mC)#(mC)#(mA) | 385 |
| E_26269 | (mA)#(mA)#(mC)(mU)(mA)(mU)(mU)(mA)(mA)(mC)(mG)(mU)(mA)(mC)(mC)(mU)#(mG)#(mA) | 386 |
| E_26277 | (mA)#(mC)#(mG)(mC)(mU)(mA)(mU)(mU)(mA)(mA)(mC)(mU)(mA)(mU)(mU)(mA)#(mA)#(mA) | 387 |
| E_26305 | (mG)#(mA)#(mA)(mU)(mA)(mC)(mC)(mA)(mC)(mG)(mA)(mA)(mA)(mG)(mC)(mA)#(mA)#(mA) | 388 |
| E_26313 | (mA)#(mC)#(mU)(mA)(mG)(mC)(mA)(mA)(mG)(mA)(mA)(mU)(mA)(mC)(mC)(mA)#(mC)#(mA) | 389 |
| E_26369 | (mU)#(mA)#(mA)(mC)(mA)(mA)(mU)(mA)(mU)(mU)(mG)(mC)(mA)(mG)(mC)(mA)#(mG)#(mA) | 390 |
| E_26374 | (mC)#(mA)#(mC)(mG)(mU)(mU)(mA)(mA)(mC)(mA)(mA)(mU)(mA)(mU)(mU)(mG)#(mC)#(mA) | 391 |
| E_26455 | (mU)#(mU)#(mA)(mG)(mA)(mC)(mC)(mA)(mG)(mA)(mA)(mG)(mA)(mU)(mC)(mA)#(mG)#(mA) | 392 |
| E_26463 | (mU)#(mA)#(mG)(mU)(mU)(mC)(mG)(mU)(mU)(mU)(mA)(mG)(mA)(mC)(mC)(mA)#(mG)#(mA) | 393 |
| E_26467 | (mU)#(mA)#(mU)(mU)(mU)(mA)(mG)(mU)(mU)(mC)(mG)(mU)(mU)(mU)(mA)(mG)#(mA)#(mA) | 394 |
| E_26470 | (mU)#(mA)#(mA)(mU)(mA)(mU)(mU)(mU)(mA)(mG)(mU)(mU)(mC)(mG)(mU)(mU)#(mU)#(mA) | 395 |
| M_26573 | (mA)#(mC)#(mU)(mA)(mG)(mG)(mU)(mU)(mC)(mC)(mA)(mU)(mU)(mG)(mU)(mU)#(mC)#(mA) | 396 |
| M_26581 | (mA)#(mA)#(mC)(mC)(mU)(mA)(mU)(mU)(mA)(mC)(mU)(mA)(mG)(mG)(mU)(mU)#(mC)#(mA) | 397 |
| M_26602 | (mA)#(mA)#(mA)(mU)(mC)(mC)(mA)(mU)(mG)(mU)(mA)(mA)(mG)(mG)(mA)(mA)#(mU)#(mA) | 398 |
| M_26624 | (mG)#(mC)#(mA)(mU)(mA)(mG)(mG)(mC)(mA)(mA)(mA)(mU)(mU)(mG)(mU)(mA)#(mG)#(mA) | 399 |
| M_26637 | (mC)#(mC)#(mU)(mA)(mU)(mU)(mC)(mC)(mU)(mG)(mU)(mU)(mG)(mG)(mC)(mA)#(mU)#(mA) | 400 |
| M_26638 | (mA)#(mC)#(mC)(mU)(mA)(mU)(mU)(mC)(mC)(mU)(mG)(mU)(mU)(mG)(mG)(mC)#(mA)#(mA) | 401 |
| M_26693 | (mG)#(mC)#(mU)(mA)(mA)(mA)(mG)(mU)(mU)(mA)(mC)(mU)(mG)(mG)(mC)(mC)#(mA)#(mA) | 402 |
| M_26717 | (mU)#(mA)#(mA)(mA)(mC)(mA)(mG)(mC)(mA)(mG)(mC)(mA)(mA)(mG)(mC)(mA)#(mC)#(mA) | 403 |
| M_27014 | (mA)#(mC)#(mA)(mG)(mU)(mG)(mA)(mU)(mU)(mU)(mC)(mU)(mU)(mU)(mA)(mG)#(mG)#(mA) | 404 |
| M_27032 | (mA)#(mG)#(mC)(mG)(mU)(mU)(mC)(mG)(mU)(mG)(mA)(mU)(mG)(mU)(mA)(mG)#(mC)#(mA) | 405 |
| M_27035 | (mG)#(mA)#(mA)(mA)(mG)(mC)(mG)(mU)(mU)(mC)(mG)(mU)(mG)(mA)(mU)(mG)#(mU)#(mA) | 406 |
| M_27123 | (mU)#(mA)#(mA)(mU)(mU)(mU)(mA)(mU)(mA)(mG)(mU)(mU)(mG)(mC)(mC)(mA)#(mA)#(mA) | 407 |
| 7a_27455 | (mU)#(mA)#(mC)(mC)(mU)(mC)(mU)(mA)(mA)(mC)(mA)(mC)(mA)(mC)(mU)(mC)#(mU)#(mA) | 408 |
| 7a_27522 | (mA)#(mG)#(mA)(mG)(mG)(mA)(mU)(mG)(mA)(mA)(mA)(mU)(mG)(mG)(mU)(mG)#(mA)#(mA) | 409 |
| 7a_27537 | (mA)#(mA)#(mU)(mU)(mU)(mG)(mU)(mU)(mA)(mU)(mC)(mA)(mG)(mC)(mU)(mA)#(mG)#(mA) | 410 |
| 7a_27553 | (mA)#(mA)#(mA)(mG)(mC)(mA)(mA)(mG)(mU)(mC)(mA)(mG)(mU)(mG)(mC)(mA)#(mA)#(mA) | 411 |
| 7a_27565 | (mA)#(mA)#(mA)(mU)(mU)(mG)(mA)(mG)(mU)(mG)(mC)(mU)(mA)(mA)(mA)(mG)#(mC)#(mA) | 412 |
| 7a_27633 | (mA)#(mG)#(mU)(mU)(mU)(mA)(mG)(mG)(mU)(mG)(mA)(mA)(mA)(mC)(mU)(mG)#(mA)#(mA) | 413 |
| 7a_27656 | (mG)#(mA)#(mA)(mC)(mU)(mU)(mC)(mC)(mU)(mC)(mU)(mU)(mG)(mU)(mC)(mU)#(mG)#(mA) | 414 |
| 7a_27671 | (mG)#(mA)#(mG)(mA)(mG)(mU)(mA)(mA)(mA)(mG)(mU)(mU)(mC)(mU)(mU)(mG)#(mA)#(mA) | 415 |
| 7a_27705 | (mA)#(mU)#(mA)(mA)(mA)(mC)(mA)(mC)(mU)(mA)(mU)(mU)(mG)(mC)(mC)(mG)#(mC)#(mA) | 416 |
| 7a_27715 | (mG)#(mC)#(mA)(mA)(mA)(mG)(mU)(mG)(mU)(mU)(mA)(mU)(mA)(mA)(mA)(mC)#(mA)#(mA) | 417 |
| 7a_27720 | (mG)#(mU)#(mG)(mA)(mA)(mG)(mC)(mA)(mA)(mA)(mG)(mU)(mG)(mU)(mU)(mA)#(mU)#(mA) | 418 |
| 7a_27751 | (mA)#(mA)#(mA)(mG)(mU)(mU)(mC)(mA)(mA)(mU)(mC)(mA)(mU)(mU)(mC)(mU)#(mG)#(mA) | 419 |
| 8b_27932 | (mU)#(mC)#(mU)(mU)(mG)(mG)(mU)(mG)(mA)(mA)(mA)(mU)(mG)(mC)(mA)(mG)#(mC)#(mA) | 420 |
| 8b_27940 | (mA)#(mA)#(mC)(mU)(mA)(mC)(mA)(mU)(mU)(mC)(mU)(mU)(mG)(mG)(mU)(mG)#(mA)#(mA) | 421 |
| 8b_27986 | (mC)#(mA)#(mC)(mG)(mG)(mG)(mU)(mC)(mA)(mU)(mC)(mA)(mA)(mC)(mU)(mA)#(mC)#(mA) | 422 |
| 8b_28002 | (mA)#(mU)#(mA)(mG)(mA)(mA)(mG)(mU)(mG)(mA)(mA)(mU)(mA)(mG)(mG)(mA)#(mC)#(mA) | 423 |
| 8b_28024 | (mC)#(mU)#(mA)(mC)(mU)(mC)(mU)(mA)(mA)(mU)(mA)(mU)(mA)(mC)(mC)(mA)#(mU)#(mA) | 424 |
| 8b_28091 | (mU)#(mG)#(mA)(mA)(mU)(mG)(mG)(mG)(mU)(mG)(mA)(mU)(mU)(mU)(mA)(mG)#(mA)#(mA) | 425 |
| 8b_28119 | (mA)#(mA)#(mC)(mU)(mG)(mU)(mA)(mU)(mA)(mA)(mU)(mU)(mA)(mC)(mC)(mG)#(mA)#(mA) | 426 |
| 8b_28127 | (mA)#(mA)#(mA)(mC)(mA)(mG)(mG)(mA)(mA)(mA)(mC)(mU)(mG)(mU)(mA)(mU)#(mA)#(mA) | 427 |
| 8b_28128 | (mU)#(mA)#(mA)(mA)(mC)(mA)(mG)(mG)(mA)(mA)(mA)(mC)(mU)(mG)(mU)(mA)#(mU)#(mA) | 428 |
| 8b_28163 | (mC)#(mC)#(mC)(mA)(mA)(mU)(mU)(mU)(mA)(mG)(mG)(mU)(mU)(mC)(mC)(mU)#(mG)#(mA) | 429 |
| 8b_28218 | (mA)#(mA)#(mC)(mG)(mU)(mC)(mA)(mU)(mG)(mA)(mU)(mA)(mC)(mU)(mC)(mU)#(mA)#(mA) | 430 |
| 8b_28222 | (mC)#(mA)#(mC)(mG)(mA)(mA)(mC)(mG)(mU)(mC)(mA)(mU)(mG)(mA)(mU)(mA)#(mC)#(mA) | 431 |
| N_28407 | (mA)#(mC)#(mG)(mC)(mA)(mG)(mU)(mA)(mU)(mU)(mA)(mU)(mU)(mG)(mG)(mG)#(mU)#(mA) | 432 |
| N_28655 | (mA)#(mA)#(mC)(mC)(mC)(mA)(mU)(mA)(mU)(mG)(mA)(mU)(mG)(mC)(mC)(mG)#(mU)#(mA) | 433 |
| N_28945 | (mA)#(mG)#(mC)(mU)(mG)(mG)(mU)(mU)(mC)(mA)(mA)(mU)(mC)(mU)(mG)(mU)#(mC)#(mA) | 434 |
| N_28992 | (mC)#(mA)#(mG)(mU)(mU)(mU)(mG)(mG)(mC)(mC)(mU)(mU)(mG)(mU)(mU)(mG)#(mU)#(mA) | 435 |
| N_29141 | (mU)#(mC)#(mC)(mU)(mU)(mG)(mU)(mC)(mU)(mG)(mA)(mU)(mU)(mA)(mG)(mU)#(mU)#(mA) | 436 |
| N_29276 | (mA)#(mU)#(mC)(mC)(mA)(mA)(mU)(mU)(mU)(mG)(mA)(mU)(mG)(mG)(mC)(mA)#(mC)#(mA) | 437 |
| N_29292 | (mA)#(mA)#(mU)(mU)(mU)(mG)(mG)(mA)(mU)(mC)(mU)(mU)(mU)(mG)(mU)(mC)#(mA)#(mA) | 438 |
| N_29293 | (mA)#(mA)#(mA)(mU)(mU)(mU)(mG)(mG)(mA)(mU)(mC)(mU)(mU)(mU)(mG)(mU)#(mC)#(mA) | 439 |
| N_29303 | (mU)#(mU)#(mG)(mA)(mU)(mC)(mU)(mU)(mU)(mG)(mA)(mA)(mA)(mU)(mU)(mU)#(mG)#(mA) | 440 |
| N_29307 | (mU)#(mG)#(mA)(mC)(mU)(mU)(mG)(mA)(mU)(mC)(mU)(mU)(mU)(mG)(mA)(mA)#(mA)#(mA) | 441 |
| N_29328 | (mC)#(mA)#(mA)(mU)(mA)(mU)(mG)(mC)(mU)(mU)(mA)(mU)(mU)(mC)(mA)(mG)#(mC)#(mA) | 442 |
| N_29464 | (mA)#(mA)#(mA)(mU)(mC)(mA)(mU)(mC)(mC)(mA)(mA)(mA)(mU)(mC)(mU)(mG)#(mC)#(mA) | 443 |

TABLE 7
SARS-CoV2-Additional target sequences

| | | SEQ | | SEQ |
|---|---|---|---|---|
| | | ID | | ID |
| Sequence ID | 20 nt Sequence | NO: | 45 nt Gene Region | NO: |
| 3a_25688 | CUGGCCUUGAAGCC | 444 | TTTTGCTCGTTGCTGCT | 642 |
| | CCUUUU | | GGCCTTGAAGCCCCTT | |
| | | | TTCTCTATCTTT | |
| 3a_25570 | UCCAAAAUCAUAAC | 445 | GTTTTTCAGAGCGCTTC | 643 |
| | CCUCAA | | CAAAATCATAACCCTC | |
| | | | AAAAAGAGATGG | |
| 3a_25852 | UACGACUAUUGUA | 446 | TGGCATACTAATTGTT | 644 |
| | UACCUUA | | ACGACTATTGTATACC | |
| | | | TTACAATAGTGTA | |
| 3a_25518 | UUUCGGAUGGCUU | 447 | ACAAGCCTCACTCCCT | 645 |
| | AUUGUUG | | TTCGGATGGCTTATTGT | |
| | | | TGGCGTTGCACT | |
| 3a_25974 | AUCUGGAGUAAAA | 448 | TACTGAAAAATGGGAA | 646 |
| | GACUGUG | | TCTGGAGTAAAAGACT | |
| | | | GTGTTGTATTACA | |
| 3a_25835 | GCUGGCAUACUAA | 449 | CCAACTATTTTCTTTGC | 647 |
| | UUGUUAC | | TGGCATACTAATTGTT | |
| | | | ACGACTATTGTA | |
| 3a_25645 | UUGUUGUUUGUAA | 450 | GTTTGCAACTTGCTGTT | 648 |
| | CAGUUUA | | GTTGTTTGTAACAGTTT | |
| | | | ACTCACACCTT | |
| 3a_25773 | UUGGCUUUGCUGG | 451 | AATAATAATGAGGCTT | 649 |
| | AAAUGCC | | TGGCTTTGCTGGAAAT | |
| | | | GCCGTTCCAAAAA | |
| 3a_25921 | AGUCCUAUUUCUG | 452 | GGTGATGGCACAACAA | 650 |
| | AACAUGA | | GTCCTATTTCTGAACAT | |
| | | | GACTACCAGATT | |
| 3a_26114 | AAGAACAUGUCCA | 453 | TTGTTGATGAGCCTGA | 651 |
| | AAUUCAC | | AGAACATGTCCAAATT | |
| | | | CACACAATCGACG | |
| 3a_25750 | UUUGUAAGAAUAA | 454 | TTGCAGAGTATAAACT | 652 |
| | UAAUGAG | | TTGTAAGAATAATAAT | |
| | | | GAGGCTTTGGCTT | |
| 3a_25862 | GUAUACCUUACAA | 455 | ATTGTTACGACTATTGT | 653 |
| | UAGUGUA | | ATACCTTACAATAGTG | |
| | | | TAACTTCTTCAA | |
| 3a_25847 | AUUGUUACGACUA | 456 | TTTGCTGGCATACTAA | 654 |
| | UUGUAUA | | TTGTTACGACTATTGTA | |
| | | | TACCTTACAATA | |
| 3a_26105 | AUGAGCCUGAAGA | 457 | ACAATAAAATTGTTGA | 655 |
| | ACAUGUC | | TGAGCCTGAAGAACAT | |
| | | | GTCCAAATTCACA | |
| 3a_25863 | UAUACCUUACAAU | 458 | TTGTTACGACTATTGTA | 656 |
| | AGUGUAA | | TACCTTACAATAGTGT | |
| | | | AACTTCTTCAAT | |
| 3a_26164 | CCAGUAAUGGAACC | 459 | TCCGGAGTTGTTAATC | 657 |
| | AAUUUA | | CAGTAATGGAACCAAT | |
| | | | TTATGATGAACCG | |
| 3a_25706 | UUCUCUAUCUUUA | 460 | GCCTTGAAGCCCCTTTT | 658 |
| | UGCUUUA | | CTCTATCTTTATGCTTT | |
| | | | AGTCTACTTCT | |
| 3a_25595 | GAUGGCAACUAGC | 461 | TAACCCTCAAAAAGAG | 659 |
| | ACUCUCC | | ATGGCAACTAGCACTC | |
| | | | TCCAAGGGTGTTC | |
| 3a_25611 | CUCCAAGGGUGUUC | 462 | ATGGCAACTAGCACTC | 660 |
| | ACUUUG | | TCCAAGGGTGTTCACT | |
| | | | TTGTTTGCAACTT | |
| 3a_25405 | AUGAGAAUCUUCA | 463 | CTTATGGATTTGTTTAT | 661 |
| | CAAUUGG | | GAGAATCTTCACAATT | |
| | | | GGAACTGTAACT | |
| 3a_25535 | UUGGCGUUGCACU | 464 | TCGGATGGCTTATTGTT | 662 |
| | UCUUGCU | | GGCGTTGCACTTCTTG | |
| | | | CTGTTTTTCAGA | |
| 3a_25653 | UGUAACAGUUUAC | 465 | CTTGCTGTTGTTGTTTG | 663 |
| | UCACACC | | TAACAGTTTACTCACA | |
| | | | CCTTTTGCTCGT | |
| 7a_27598 | GACGGCGUAAAAC | 466 | GCTTTTGCTTGTCCTGA | 664 |
| | ACGUCUA | | CGGCGTAAAACACGTC | |
| | | | TATCAGTTACGT | |
| 7a_27483 | UUUAAAAGAACCU | 467 | AGGTACAACAGTACTT | 665 |
| | UGCUCUU | | TTAAAAGAACCTTGCT | |
| | | | CTTCTGGAACATA | |
| 7a_27258 | GAGGACUUUUAAA | 468 | ATTACTAATTATTATG | 666 |
| | GUUUCCA | | AGGACTTTTAAAGTTT | |
| | | | CCATTTGGAATCT | |
| 7a_27264 | UUUUAAAGUUUCC | 469 | AATTATTATGAGGACT | 667 |
| | AUUUGGA | | TTTAAAGTTTCCATTTG | |
| | | | GAATCTTGATTA | |
| 7a_27257 | UGAGGACUUUUAA | 470 | TATTACTAATTATTATG | 668 |
| | AGUUUCC | | AGGACTTTTAAAGTTT | |
| | | | CCATTTGGAATC | |
| 7a_27334 | ACUGAGAAUAAAU | 471 | TTATCTAAGTCACTAA | 669 |
| | AUUCUCA | | CTGAGAATAAATATTC | |
| | | | TCAATTAGATGAA | |
| 7a_27595 | CCUGACGGCGUAAA | 472 | TTTGCTTTTGCTTGTCC | 670 |
| | ACACGU | | TGACGGCGTAAAACAC | |
| | | | GTCTATCAGTTA | |
| 7a_27466 | AGAGGUACAACAG | 473 | TACCAAGAGTGTGTTA | 671 |
| | UACUUUU | | GAGGTACAACAGTACT | |
| | | | TTTAAAAGAACCT | |
| 7a_27237 | AGAGAUAUUACUA | 474 | TCAGGTTACTATAGCA | 672 |
| | AUUAUUA | | GAGATATTACTAATTA | |
| | | | TTATGAGGACTTT | |
| 7a_27395 | UGAAAAUUAUUCU | 475 | TTGATTAAACGAACAT | 673 |
| | UUUCUUG | | GAAAATTATTCTTTTCT | |
| | | | TGGCACTGATAA | |
| 7a_27289 | GAUUACAUCAUAA | 476 | TCCATTTGGAATCTTG | 674 |
| | ACCUCAU | | ATTACATCATAAACCT | |
| | | | CATAATTAAAAAT | |
| 7a_27243 | AUUACUAAUUAUU | 477 | TACTATAGCAGAGATA | 675 |
| | AUGAGGA | | TTACTAATTATTATGA | |
| | | | GGACTTTTAAAGT | |
| 7a_27319 | UUAUCUAAGUCAC | 478 | CTCATAATTAAAAATT | 676 |
| | UAACUGA | | TATCTAAGTCACTAAC | |
| | | | TGAGAATAAATAT | |
| 7a_27226 | GUUACUAUAGCAG | 479 | CTCGTTGACTTTCAGGT | 677 |
| | AGAUAUU | | TACTATAGCAGAGATA | |
| | | | TTACTAATTATT | |
| 7a_27256 | AUGAGGACUUUUA | 480 | ATATTACTAATTATTAT | 678 |
| | AAGUUUC | | GAGGACTTTTAAAGTT | |
| | | | TCCATTTGGAAT | |
| 7a_27203 | UGUUUCAUCUCGU | 481 | GTAAGTGACAACAGAT | 679 |
| | UGACUUU | | GTTTCATCTCGTTGACT | |
| | | | TTCAGGTTACTA | |
| 7a_27292 | UACAUCAUAAACCU | 482 | ATTTGGAATCTTGATT | 680 |
| | CAUAAU | | ACATCATAAACCTCAT | |
| | | | AATTAAAAATTTA | |
| 7a_27333 | AACUGAGAAUAAA | 483 | TTTATCTAAGTCACTA | 681 |
| | UAUUCUC | | ACTGAGAATAAATATT | |
| | | | CTCAATTAGATGA | |
| 7a_27255 | UAUGAGGACUUUU | 484 | GATATTACTAATTATT | 682 |
| | AAAGUUU | | ATGAGGACTTTTAAAG | |
| | | | TTTCCATTTGGAA | |
| 7a_27381 | UGAUUAAACGAAC | 485 | GCAACCAATGGAGATT | 683 |
| | AUGAAAA | | GATTAAACGAACATGA | |
| | | | AAATTATTCTTTT | |
| 7a_27750 | GACAGAAUGAUUG | 486 | CACACTCAAAAGAAAG | 684 |
| | AACUUUC | | ACAGAATGATTGAACT | |
| | | | TTCATTAATTGAC | |
| 7a_27445 | UAUCACUACCAAGA | 487 | GCTACTTGTGAGCTTT | 685 |
| | GUGUGU | | ATCACTACCAAGAGTG | |
| | | | TGTTAGAGGTACA | |
| 8b_27795 | UUAGCCUUUCUGCU | 488 | TTCTATTTGTGCTTTTT | 686 |
| | AUUCCU | | AGCCTTTCTGCTATTCC | |
| | | | TTGTTTTAATT | |
| 8b_27803 | UCUGCUAUUCCUUG | 489 | GTGCTTTTTAGCCTTTC | 687 |
| | UUUUAA | | TGCTATTCCTTGTTTTA | |
| | | | ATTATGCTTAT | |
| 8b_27888 | ACGAACAUGAAAU | 490 | ACTTGTCACGCCTAAA | 688 |
| | UUCUUGU | | CGAACATGAAATTTCT | |
| | | | TGTTTTCTTAGGA | |
| 8b_28236 | CGUGUUGUUUUAG | 491 | GAGTATCATGACGTTC | 689 |
| | AUUUCAU | | GTGTTGTTTTAGATTTC | |
| | | | ATCTAAACGAAC | |
| 8b_27895 | UGAAAUUUCUUGU | 492 | ACGCCTAAACGAACAT | 690 |
| | UUUCUUA | | GAAATTTCTTGTTTTCT | |
| | | | TAGGAATCATCA | |
| 8b_28142 | UUUACCUUUUACA | 493 | TTATACAGTTTCCTGTT | 691 |
| | AUUAAUU | | TACCTTTTACAATTAAT | |
| | | | TGCCAGGAACC | |
| 8b_27802 | UUCUGCUAUUCCUU | 494 | TGTGCTTTTTAGCCTTT | 692 |
| | GUUUUA | | CTGCTATTCCTTGTTTT | |
| | | | AATTATGCTTA | |
| 8b_27808 | UAUUCCUUGUUUU | 495 | TTTTAGCCTTTCTGCTA | 693 |
| | AAUUAUG | | TTCCTTGTTTTAATTAT | |
| | | | GCTTATTATCT | |
| 8b_27796 | UAGCCUUUCUGCUA | 496 | TCTATTTGTGCTTTTTA | 694 |
| | UUCCUU | | GCCTTTCTGCTATTCCT | |
| | | | TGTTTTAATTA | |
| 8b_27815 | UGUUUUAAUUAUG | 497 | CTTTCTGCTATTCCTTG | 695 |
| | CUUAUUA | | TTTTAATTATGCTTATT | |
| | | | ATCTTTTGGTT | |
| 8b_28044 | GCUAGAAAAUCAG | 498 | TATATTAGAGTAGGAG | 696 |
| | CACCUUU | | CTAGAAAATCAGCACC | |
| | | | TTTAATTGAATTG | |
| 8b_27794 | UUUAGCCUUUCUGC | 499 | CTTCTATTTGTGCTTTT | 697 |
| | UAUUCC | | TAGCCTTTCTGCTATTC | |
| | | | CTTGTTTTAAT | |
| 8b_28234 | UUCGUGUUGUUUU | 500 | TAGAGTATCATGACGT | 698 |
| | AGAUUUC | | TCGTGTTGTTTTAGATT | |
| | | | TCATCTAAACGA | |
| 8b_27817 | UUUUAAUUAUGCU | 501 | TTCTGCTATTCCTTGTT | 699 |
| | UAUUAUC | | TTAATTATGCTTATTAT | |
| | | | CTTTTGGTTCT | |
| 8b_27970 | CUCAACAUCAACCA | 502 | GTTTACAGTCATGTAC | 700 |
| | UAUGUA | | TCAACATCAACCATAT | |
| | | | GTAGTTGATGACC | |
| 8b_28043 | AGCUAGAAAAUCA | 503 | GTATATTAGAGTAGGA | 701 |
| | GCACCUU | | GCTAGAAAATCAGCAC | |
| | | | CTTTAATTGAATT | |
| 8b_28219 | UAGAGUAUCAUGA | 504 | TCTATGAAGACTTTTTA | 702 |
| | CGUUCGU | | GAGTATCATGACGTTC | |
| | | | GTGTTGTTTTAG | |
| 8b_27764 | ACUUUCAUUAAUU | 505 | AGACAGAATGATTGAA | 703 |
| | GACUUCU | | CTTTCATTAATTGACTT | |
| | | | CTATTTGTGCTT | |
| 8b_27963 | UCAUGUACUCAACA | 506 | GAATGTAGTTTACAGT | 704 |
| | UCAACC | | CATGTACTCAACATCA | |
| | | | ACCATATGTAGTT | |
| 8b_28104 | AUUCAGUACAUCG | 507 | GGTTCTAAATCACCCA | 705 |
| | AUAUCGG | | TTCAGTACATCGATAT | |
| | | | CGGTAATTATACA | |
| 8b_28018 | AUUCUAAAUGGUA | 503 | GTCCTATTCACTTCTAT | 706 |
| | UAUUAGA | | TCTAAATGGTATATTA | |
| | | | GAGTAGGAGCTA | |
| 8b_27892 | ACAUGAAAUUUCU | 509 | GTCACGCCTAAACGAA | 707 |
| | UGUUUUC | | CATGAAATTTCTTGTTT | |
| | | | TCTTAGGAATCA | |
| E_26390 | UGAGUCUUGUAAA | 510 | GCAATATTGTTAACGT | 708 |
| | ACCUUCU | | GAGTCTTGTAAAACCT | |
| | | | TCTTTTTACGTTT | |
| E_26391 | GAGUCUUGUAAAA | 511 | CAATATTGTTAACGTG | 709 |
| | CCUUCUU | | AGTCTTGTAAAACCTT | |
| | | | CTTTTTACGTTTA | |
| E_26392 | AGUCUUGUAAAAC | 512 | AATATTGTTAACGTGA | 710 |
| | CUUCUUU | | GTCTTGTAAAACCTTCT | |
| | | | TTTTACGTTTAC | |
| E_26468 | UCUAAACGAACUA | 513 | TTCCTGATCTTCTGGTC | 711 |
| | AAUAUUA | | TAAACGAACTAAATAT | |
| | | | TATATTAGTTTT | |
| E_26321 | UCUUGCUAGUUAC | 514 | TTGCTTTCGTGGTATTC | 712 |
| | ACUAGCC | | TTGCTAGTTACACTAG | |
| | | | CCATCCTTACTG | |
| E_26259 | UUCGGAAGAGACA | 515 | TATGTACTCATTCGTTT | 713 |
| | GGUACGU | | CGGAAGAGACAGGTAC | |
| | | | GTTAATAGTTAA | |
| E_26460 | UCUUCUGGUCUAA | 516 | TTCTAGAGTTCCTGATC | 714 |
| | ACGAACU | | TTCTGGTCTAAACGAA | |
| | | | CTAAATATTATA | |
| E_26284 | GUUAAUAGCGUAC | 517 | ACAGGTACGTTAATAG | 715 |
| | UUCUUUU | | TTAATAGCGTACTTCTT | |
| | | | TTTCTTGCTTTC | |
| E_26314 | GUGGUAUUCUUGC | 518 | CTTTTTCTTGCTTTCGT | 716 |
| | UAGUUAC | | GGTATTCTTGCTAGTTA | |
| | | | CACTAGCCATC | |
| E_26274 | UACGUUAAUAGUU | 519 | TTCGGAAGAGACAGGT | 717 |
| | AAUAGCG | | ACGTTAATAGTTAATA | |
| | | | GCGTACTTCTTTT | |
| E_26260 | UCGGAAGAGACAG | 520 | ATGTACTCATTCGTTTC | 718 |
| | GUACGUU | | GGAAGAGACAGGTAC | |
| | | | GTTAATAGTTAAT | |
| E_26308 | GCUUUCGUGGUAU | 521 | GTACTTCTTTTTCTTGC | 719 |
| | UCUUGCU | | TTTCGTGGTATTCTTGC | |
| | | | TAGTTACACTA | |
| E_26411 | UUUACGUUUACUC | 522 | TTGTAAAACCTTCTTTT | 720 |
| | UCGUGUU | | TACGTTTACTCTCGTGT | |
| | | | TAAAAATCTGA | |
| E_26265 | AGAGACAGGUACG | 523 | CTCATTCGTTTCGGAA | 721 |
| | UUAAUAG | | GAGACAGGTACGTTAA | |
| | | | TAGTTAATAGCGT | |
| E_26279 | UAAUAGUUAAUAG | 524 | AAGAGACAGGTACGTT | 722 |
| | CGUACUU | | AATAGTTAATAGCGTA | |
| | | | CTTCTTTTTCTTG | |
| E_26312 | UCGUGGUAUUCUU | 525 | TTCTTTTTCTTGCTTTC | 723 |
| | GCUAGUU | | GTGGTATTCTTGCTAGT | |
| | | | TACACTAGCCA | |
| E_26464 | CUGGUCUAAACGA | 526 | AGAGTTCCTGATCTTCT | 724 |
| | ACUAAAU | | GGTCTAAACGAACTAA | |
| | | | ATATTATATTAG | |
| E_26231 | AUGAGUACGAACU | 527 | TGTAAGCACAAGCTGA | 725 |
| | UAUGUAC | | TGAGTACGAACTTATG | |
| | | | TACTCATTCGTTT | |
| E_26469 | CUAAACGAACUAA | 528 | TCCTGATCTTCTGGTCT | 726 |
| | AUAUUAU | | AAACGAACTAAATATT | |
| | | | ATATTAGTTTTT | |
| E_26413 | UACGUUUACUCUCG | 529 | GTAAAACCTTCTTTTTA | 727 |
| | UGUUAA | | CGTTTACTCTCGTGTTA | |
| | | | AAAATCTGAAT | |
| E_26281 | AUAGUUAAUAGCG | 530 | GAGACAGGTACGTTAA | 728 |
| | UACUUCU | | TAGTTAATAGCGTACT | |
| | | | TCTTTTTCTTGCT | |
| E_26462 | UUCUGGUCUAAAC | 531 | CTAGAGTTCCTGATCTT | 729 |
| | GAACUAA | | CTGGTCTAAACGAACT | |
| | | | AAATATTATATT | |
| M_26517 | UUAGCCAUGGCAG | 532 | TTTGGAACTTTAATTTT | 730 |
| | AUUCCAA | | AGCCATGGCAGATTCC | |
| | | | AACGGTACTATT | |
| M_26522 | CAUGGCAGAUUCCA | 533 | AACTTTAATTTTAGCC | 731 |
| | ACGGUA | | ATGGCAGATTCCAACG | |
| | | | GTACTATTACCGT | |
| M_26656 | UUUUGUAUAUAAU | 534 | CCAACAGGAATAGGTT | 732 |
| | UAAGUUA | | TTTGTATATAATTAAGT | |
| | | | TAATTTTCCTCT | |
| M_26603 | AUUCCUUACAUGG | 535 | AGTAATAGGTTTCCTA | 733 |
| | AUUUGUC | | TTCCTTACATGGATTTG | |
| | | | TCTTCTACAATT | |
| M_26572 | UUGAACAAUGGAA | 536 | AGCTTAAAAAGCTCCT | 734 |
| | CCUAGUA | | TGAACAATGGAACCTA | |
| | | | GTAATAGGTTTCC | |
| M_27101 | UGCUGCAUACAGUC | 537 | AGGTGACTCAGGTTTT | 735 |
| | GCUACA | | GCTGCATACAGTCGCT | |
| | | | ACAGGATTGGCAA | |
| M_27049 | UUUCUUAUUACAA | 538 | CTACATCACGAACGCT | 736 |
| | AUUGGGA | | TTCTTATTACAAATTGG | |
| | | | GAGCTTCGCAGC | |
| M_26580 | UGGAACCUAGUAA | 539 | AAGCTCCTTGAACAAT | 737 |
| | UAGGUUU | | GGAACCTAGTAATAGG | |
| | | | TTTCCTATTCCTT | |
| M_26733 | UACAGAAUAAAUU | 540 | GTGCTTGCTGCTGTTTA | 738 |
| | GGAUCAC | | CAGAATAAATTGGATC | |
| | | | ACCGGTGGAATT | |
| M_26495 | UUUUCUGUUUGGA | 541 | AAATATTATATTAGTTT | 739 |
| | ACUUUAA | | TTCTGTTTGGAACTTTA | |
| | | | ATTTTAGCCAT | |
| M_26608 | UUACAUGGAUUUG | 542 | TAGGTTTCCTATTCCTT | 740 |
| | UCUUCUA | | ACATGGATTTGTCTTCT | |
| | | | ACAATTTGCCT | |
| M_26590 | UAAUAGGUUUCCU | 543 | AACAATGGAACCTAGT | 741 |
| | AUUCCUU | | AATAGGTTTCCTATTCC | |
| | | | TTACATGGATTT | |
| M_26501 | GUUUGGAACUUUA | 544 | TATATTAGTTTTTCTGT | 742 |
| | AUUUUAG | | TTGGAACTTTAATTTTA | |
| | | | GCCATGGCAGA | |
| M_26796 | UGGCUCAGCUACUU | 545 | CTTGTAGGCTTGATGT | 743 |
| | CAUUGC | | GGCTCAGCTACTTCAT | |
| | | | TGCTTCTTTCAGA | |
| M_26570 | CCUUGAACAAUGG | 546 | AGAGCTTAAAAAGCTC | 744 |
| | AACCUAG | | CTTGAACAATGGAACC | |
| | | | TAGTAATAGGTTT | |
| M_27124 | UUGGCAACUAUAA | 547 | ACAGTCGCTACAGGAT | 745 |
| | AUUAAAC | | TGGCAACTATAAATTA | |
| | | | AACACAGACCATT | |
| M_26814 | GCUUCUUUCAGACU | 548 | CTCAGCTACTTCATTGC | 746 |
| | GUUUGC | | TTCTTTCAGACTGTTTG | |
| | | | CGCGTACGCGT | |
| M_26694 | UGGCCAGUAACUU | 549 | TTCCTCTGGCTGTTATG | 747 |
| | UAGCUUG | | GCCAGTAACTTTAGCT | |
| | | | TGTTTTGTGCTT | |
| M_27047 | GCUUUCUUAUUAC | 550 | TGCTACATCACGAACG | 748 |
| | AAAUUGG | | CTTTCTTATTACAAATT | |
| | | | GGGAGCTTCGCA | |
| M_27153 | UCCAGUAGCAGUG | 551 | TTAAACACAGACCATT | 749 |
| | ACAAUAU | | CCAGTAGCAGTGACAA | |
| | | | TATTGCTTTGCTT | |
| M_27163 | GUGACAAUAUUGC | 552 | ACCATTCCAGTAGCAG | 750 |
| | UUUGCUU | | TGACAATATTGCTTTG | |
| | | | CTTGTACAGTAAG | |
| M_26473 | ACGAACUAAAUAU | 553 | GATCTTCTGGTCTAAA | 751 |
| | UAUAUUA | | CGAACTAAATATTATA | |
| | | | TTAGTTTTTCTGT | |
| N_29327 | UUGCUGAAUAAGC | 554 | AAAGATCAAGTCATTT | 752 |
| | AUAUUGA | | TGCTGAATAAGCATAT | |
| | | | TGACGCATACAAA | |
| N_29216 | UUCGGAAUGUCGC | 555 | AGCGCTTCAGCGTTCT | 753 |
| | GCAUUGG | | TCGGAATGTCGCGCAT | |
| | | | TGGCATGGAAGTC | |
| N_28486 | CGUUCCAAUUAACA | 556 | CCCTCGAGGACAAGGC | 754 |
| | CCAAUA | | GTTCCAATTAACACCA | |
| | | | ATAGCAGTCCAGA | |
| N_28755 | UUCCUCAAGGAACA | 557 | CAATCGTGCTACAACT | 755 |
| | ACAUUG | | TCCTCAAGGAACAACA | |
| | | | TTGCCAAAAGGCT | |
| N_28585 | CAGUCCAAGAUGG | 558 | TAAAATGAAAGATCTC | 756 |
| | UAUUUCU | | AGTCCAAGATGGTATT | |
| | | | TCTACTACCTAGG | |
| N_28565 | GACGGUAAAAUGA | 559 | CGAATTCGTGGTGGTG | 757 |
| | AAGAUCU | | ACGGTAAAATGAAAGA | |
| | | | TCTCAGTCCAAGA | |
| N_28961 | CUUGAGAGCAAAA | 560 | GACAGATTGAACCAGC | 758 |
| | UGUCUGG | | TTGAGAGCAAAATGTC | |
| | | | TGGTAAAGGCCAA | |
| N_29310 | UCAAAGAUCAAGU | 561 | ACAAAGATCCAAATTT | 759 |
| | CAUUUUG | | CAAAGATCAAGTCATT | |
| | | | TTGCTGAATAAGC | |
| N_29177 | CCGCAAAUUGCACA | 562 | GATTACAAACATTGGC | 760 |
| | AUUUGC | | CGCAAATTGCACAATT | |
| | | | TGCCCCCAGCGCT | |
| N_28261 | CGAACAAACUAAA | 563 | TAGATTTCATCTAAAC | 761 |
| | AUGUCUG | | GAACAAACTAAAATGT | |
| | | | CTGATAATGGACC | |
| N_28476 | GAGGACAAGGCGU | 564 | ACCTTAAATTCCCTCG | 762 |
| | UCCAAUU | | AGGACAAGGCGTTCCA | |
| | | | ATTAACACCAATA | |
| N_29130 | UUGGGGACCAGGA | 565 | AAACCCAAGGAAATTT | 763 |
| | ACUAAUC | | TGGGGACCAGGAACTA | |
| | | | ATCAGACAAGGAA | |
| N_29455 | UCUUCCUGCUGCAG | 566 | GCAAACTGTGACTCTT | 764 |
| | AUUUGG | | CTTCCTGCTGCAGATTT | |
| | | | GGATGATTTCTC | |
| N_29465 | GCAGAUUUGGAUG | 567 | ACTCTTCTTCCTGCTGC | 765 |
| | AUUUCUC | | AGATTTGGATGATTTC | |
| | | | TCCAAACAATTG | |
| N_29132 | GGGGACCAGGAAC | 568 | ACCCAAGGAAATTTTG | 766 |
| | UAAUCAG | | GGGACCAGGAACTAAT | |
| | | | CAGACAAGGAACT | |
| N_29148 | UCAGACAAGGAAC | 569 | GGGACCAGGAACTAAT | 767 |
| | UGAUUAC | | CAGACAAGGAACTGAT | |
| | | | TACAAACATTGGC | |
| N_29291 | GAUGACAAAGAUC | 570 | GGTGCCATCAAATTGG | 768 |
| | CAAAUUU | | ATGACAAAGATCCAAA | |
| | | | TTTCAAAGATCAA | |
| N_29342 | AUUGACGCAUACA | 571 | TTGCTGAATAAGCATA | 769 |
| | AAACAUU | | TTGACGCATACAAAAC | |
| | | | ATTCCCACCAACA | |
| N_28589 | CCAAGAUGGUAUU | 572 | ATGAAAGATCTCAGTC | 770 |
| | UCUACUA | | CAAGATGGTATTTCTA | |
| | | | CTACCTAGGAACT | |
| N_29133 | GGGACCAGGAACU | 573 | CCCAAGGAAATTTTGG | 771 |
| | AAUCAGA | | GGACCAGGAACTAATC | |
| | | | AGACAAGGAACTG | |
| N_29057 | CGUACUGCCACUAA | 574 | AAGCCTCGGCAAAAAC | 772 |
| | AGCAUA | | GTACTGCCACTAAAGC | |
| | | | ATACAATGTAACA | |
| N_28682 | GGAGCCUUGAAUA | 575 | TGGGTTGCAACTGAGG | 773 |
| | CACCAAA | | GAGCCTTGAATACACC | |
| | | | AAAAGATCACATT | |
| orf1a_7249 | UUUGGCAUAUAUU | 576 | AGTTGCAGAGTGGTTT | 774 |
| | CUUUUCA | | TTGGCATATATTCTTTT | |
| | | | CACTAGGTTTTT | |
| orf1a_1066 | UGGGGAAUGUCCA | 577 | ATTTGACACCTTCAAT | 775 |
| | AAUUUUG | | GGGGAATGTCCAAATT | |
| | | | TTGTATTTCCCTT | |
| orf1a_11197 | UGCCACUGUAGCUU | 578 | TTTGTTACCTTCTCTTG | 776 |
| | AUUUUA | | CCACTGTAGCTTATTTT | |
| | | | AATATGGTCTA | |
| orf1a_13292 | UGUGACUUAAAAG | 579 | AATCCTAAAGGATTTT | 777 |
| | GUAAGUA | | GTGACTTAAAAGGTAA | |
| | | | GTATGTACAAATA | |
| orf1a_12931 | UCCUAAAGUGAAG | 580 | AGACACACCTAAAGGT | 778 |
| | UAUUUAU | | CCTAAAGTGAAGTATT | |
| | | | TATACTTTATTAA | |
| orf1a_4598 | CUUGUUACAAUGCC | 581 | GATCTAAATGAAACTC | 779 |
| | ACUUGG | | TTGTTACAATGCCACTT | |
| | | | GGCTATGTAACA | |
| orf1a_12515 | UGUGAUGGUACAA | 582 | ACATATAAAAATACGT | 780 |
| | CAUUUAC | | GTGATGGTACAACATT | |
| | | | TACTTATGCATCA | |
| orf1a_9060 | AUUGUUAUGAUAC | 583 | GTAAGCCAGTACCATA | 781 |
| | CAAUGUA | | TTGTTATGATACCAAT | |
| | | | GTACTAGAAGGTT | |
| orf1a_1286 | UGCGAAUUUUGUG | 584 | TTTGTTAAAGCCACTT | 782 |
| | GCACUGA | | GCGAATTTTGTGGCAC | |
| | | | TGAGAATTTGACT | |
| orf1a_9707 | AUUUCCACAAAGC | 585 | GCTTATATCATTTGTAT | 783 |
| | AUUUCUA | | TTCCACAAAGCATTTC | |
| | | | TATTGGTTCTTT | |
| orf1a_5110 | UAAAACAUUUUAU | 586 | TAATTCACATGAAGGT | 784 |
| | GUUUUAC | | AAAACATTTTATGTTTT | |
| | | | ACCTAATGATGA | |
| orf1a_9041 | UCUGGUAAGCCAG | 587 | ATTTTTAAAGATGCTTC | 785 |
| | UACCAUA | | TGGTAAGCCAGTACCA | |
| | | | TATTGTTATGAT | |
| orf1a_11687 | CUUGGUGUUUAUG | 588 | TACTTTAGACTGACTCT | 786 |
| | AUUACUU | | TGGTGTTTATGATTACT | |
| | | | TAGTTTCTACA | |
| orf1a_12215 | UUGAAUGUGGCUA | 589 | AAGTTGAAGAAGTCTT | 787 |
| | AAUCUGA | | TGAATGTGGCTAAATC | |
| | | | TGAATTTGACCGT | |
| orf1a_11254 | UAUGACAUGGUUG | 590 | TTGGGTGATGCGTATT | 788 |
| | GAUAUGG | | ATGACATGGTTGGATA | |
| | | | TGGTTGATACTAG | |
| orf1a_6963 | UUUUACUAUUAAG | 591 | ATATTATAATTTGGTTT | 789 |
| | UGUUUGC | | TTACTATTAAGTGTTTG | |
| | | | CCTAGGTTCTT | |
| orf1a_2969 | AUGGCUACAUACU | 592 | TTAGATGAGTGGAGTA | 790 |
| | ACUUAUU | | TGGCTACATACTACTT | |
| | | | ATTTGATGAGTCT | |
| orf1a_5449 | UAGAGAAACAAUG | 593 | TGAGTTAGGTGATGTT | 791 |
| | AGUUACU | | AGAGAAACAATGAGTT | |
| | | | ACTTGTTTCAACA | |
| orf1a_5238 | ACCCACAAGUUAAU | 594 | CTAAAAAGTGGAAATA | 792 |
| | GGUUUA | | CCCACAAGTTAATGGT | |
| | | | TTAACTTCTATTA | |
| orf1a_8059 | UUCAUCAACUUUU | 595 | TTACGTTAATACGTTTT | 793 |
| | AACGUAC | | CATCAACTTTTAACGT | |
| | | | ACCAATGGAAAA | |
| orf1a_12276 | UGGCUGAUCAAGC | 596 | GTAAGTTGGAAAAGAT | 794 |
| | UAUGACC | | GGCTGATCAAGCTATG | |
| | | | ACCCAAATGTATA | |
| orf1a_4097 | UUCUUAAAGAAAG | 597 | GACATTGACATCACTT | 795 |
| | AUGCUCC | | TCTTAAAGAAAGATGC | |
| | | | TCCATATATAGTG | |
| orf1ab_19120 | UAUAAAAUAGAAG | 598 | TGTAGTGACAAAGCTT | 796 |
| | AAUUAUU | | ATAAAATAGAAGAATT | |
| | | | ATTCTATTCTTAT | |
| orf1ab_17193 | GAAGGCAUUAAAA | 599 | TGATGCACTATGTGAG | 797 |
| | UAUUUGC | | AAGGCATTAAAATATT | |
| | | | TGCCTATAGATAA | |
| orf1ab_17034 | UGCAAAUUAUCAA | 600 | GTTTTCTAGCAATGTTG | 798 |
| | AAGGUUG | | CAAATTATCAAAAGGT | |
| | | | TGGTATGCAAAA | |
| orf1ab_19748 | UUGAAAAUAAAAC | 601 | TTGATGTAGAATTGTTT | 799 |
| | AACAUUA | | GAAAATAAAACAACAT | |
| | | | TACCTGTTAATG | |
| orf1ab_17980 | UAUGACAAGUUGC | 602 | TCTGATAGAGACCTTT | 800 |
| | AAUUUAC | | ATGACAAGTTGCAATT | |
| | | | TACAAGTCTTGAA | |
| orf1ab_21482 | UUAGUAAAGGUAG | 603 | TGATTTTATCTCTTCTT | 801 |
| | ACUUAUA | | AGTAAAGGTAGACTTA | |
| | | | TAATTAGAGAAA | |
| orf1ab_13842 | UGAUGAAGGUAAU | 604 | TGCTTTAAGGCATTTTG | 802 |
| | UGUGACA | | ATGAAGGTAATTGTGA | |
| | | | CACATTAAAAGA | |
| orf1ab_19071 | UGAUGUAGAAUGG | 605 | GTGTGTACCTCAAGCT | 803 |
| | AAGUUCU | | GATGTAGAATGGAAGT | |
| | | | TCTATGATGCACA | |
| orf1ab_13878 | UGUCACAUACAAU | 606 | ATTAAAAGAAATACTT | 804 |
| | UGUUGUG | | GTCACATACAATTGTT | |
| | | | GTGATGATGATTA | |
| orf1ab_16555 | AAUGCAAUUGCAA | 607 | AATGTTACTGACTTTA | 805 |
| | CAUGUGA | | ATGCAATTGCAACATG | |
| | | | TGACTGGACAAAT | |
| orf1ab_17284 | UCAACAUUAGAAC | 608 | AAATTCAAAGTGAATT | 806 |
| | AGUAUGU | | CAACATTAGAACAGTA | |
| | | | TGTCTTTTGTACT | |
| orf1ab_17852 | CAGAAUAUGACUA | 609 | ATTCATCACAGGGCTC | 807 |
| | UGUCAUA | | AGAATATGACTATGTC | |
| | | | ATATTCACTCAAA | |
| orf1ab_17671 | GUUAUCACGCAUG | 610 | ATGTTTTATAAGGGTG | 808 |
| | AUGUUUC | | TTATCACGCATGATGT | |
| | | | TTCATCTGCAATT | |
| orf1ab_21331 | CAUGCAAAUUACA | 611 | GATGGTTATGTCATGC | 809 |
| | UAUUUUG | | ATGCAAATTACATATT | |
| | | | TTGGAGGAATACA | |
| orf1ab_18612 | GUUGACAUCUAUG | 612 | GGCACATGGCTTTGAG | 810 |
| | AAGUAUU | | TTGACATCTATGAAGT | |
| | | | ATTTTGTGAAAAT | |
| orf1ab_19345 | UUUGUUAAUUUAA | 613 | TTTGATAAAAGTGCTT | 811 |
| | AACAAUU | | TTGTTAATTTAAAACA | |
| | | | ATTACCATTTTTC | |
| orf1ab_15409 | AAUGAGUGUGCUC | 614 | TTCTATAGATTAGCTA | 812 |
| | AAGUAUU | | ATGAGTGTGCTCAAGT | |
| | | | ATTGAGTGAAATG | |
| orf1ab_19617 | UCAGAGUUUAGAA | 615 | CACTTTTACAAGACTT | 813 |
| | AAUGUGG | | CAGAGTTTAGAAAATG | |
| | | | TGGCTTTTAATGT | |
| orf1ab_21444 | AAAAGAAGGUCAA | 616 | TGCTGTTATGTCTTTA | 814 |
| | AUCAAUG | | AAAGAAGGTCAAATCA | |
| | | | ATGATATGATTTT | |
| orf1ab_20511 | AUUACUUGAUGAU | 617 | TTCTGTTATTGATTTA | 815 |
| | UUUGUUG | | TTACTTGATGATTTTG | |
| | | | TTGAAATAATAAA | |
| orf1ab_21174 | AGAACAUUCUUGG | 618 | GGCTATAAAGATAACA | 816 |
| | AAUGCUG | | GAACATTCTTGGAATG | |
| | | | CTGATCTTTATAA | |
| orf1ab_19732 | GUUGAUGUAGAAU | 619 | ACAAAAGTTGATGGTG | 817 |
| | UGUUUGA | | TTGATGTAGAATTGTT | |
| | | | TGAAAATAAAACA | |
| S_24473 | GGUGCAAUUUCAA | 620 | CTTAGCTCCAATTTTGG | 818 |
| | GUGUUUU | | TGCAATTTCAAGTGTTT | |
| | | | TAAATGATATC | |
| S_22368 | AUCUUCAACCUAGG | 621 | CTTATTATGTGGGTTAT | 819 |
| | ACUUUU | | CTTCAACCTAGGACTT | |
| | | | TTCTATTAAAAT | |
| S_24635 | GCUGCUACUAAAA | 622 | GCTTCTGCTAATCTTGC | 820 |
| | UGUCAGA | | TGCTACTAAAATGTCA | |
| | | | GAGTGTGTACTT | |
| S_21952 | CUGUGAAUUUCAA | 623 | TGTTGTTATTAAAGTCT | 821 |
| | UUUUGUA | | GTGAATTTCAATTTTGT | |
| | | | AATGATCCATT | |
| S_23698 | UGCCAUACCCACAA | 624 | CTCTAATAACTCTATTG | 822 |
| | AUUUUA | | CCATACCCACAAATTT | |
| | | | TACTATTAGTGT | |
| S_23936 | CCACCAAUUAAAGA | 625 | CAAATTTACAAAACAC | 823 |
| | UUUUGG | | CACCAATTAAAGATTT | |
| | | | TGGTGGTTTTAAT | |
| S_22807 | UGGAAAGAUUGCU | 626 | CGCTCCAGGGCAAACT | 824 |
| | GAUUAUA | | GGAAAGATTGCTGATT | |
| | | | ATAATTATAAATT | |
| S_21938 | GUUGUUAUUAAAG | 627 | AATAACGCTACTAATG | 825 |
| | UCUGUGA | | TTGTTATTAAAGTCTGT | |
| | | | GAATTTCAATTT | |
| S_22138 | UGUGUUUAAGAAU | 628 | AAATCTTAGGGAATTT | 826 |
| | AUUGAUG | | GTGTTTAAGAATATTG | |
| | | | ATGGTTATTTTAA | |
| S_24828 | UUCCUCGUGAAGG | 629 | ATGGAAAAGCACACTT | 827 |
| | UGUCUUU | | TCCTCGTGAAGGTGTC | |
| | | | TTTGTTTCAAATG | |
| S_23315 | CUUGACAUUACACC | 630 | CAGACACTTGAGATTC | 828 |
| | AUGUUC | | TTGACATTACACCATG | |
| | | | TTCTTTTGGTGGT | |
| S_25376 | UACACAUAAACGA | 631 | GGAGTCAAATTACATT | 829 |
| | ACUUAUG | | ACACATAAACGAACTT | |
| | | | ATGGATTTGTTTA | |
| S_22259 | AUUAACAUCACUA | 632 | GATTTGCCAATAGGTA | 830 |
| | GGUUUCA | | TTAACATCACTAGGTT | |
| | | | TCAAACTTTACTT | |
| S_22129 | UAGGGAAUUUGUG | 633 | TAATTTCAAAAATCTT | 831 |
| | UUUAAGA | | AGGGAATTTGTGTTTA | |
| | | | AGAATATTGATGG | |
| S_24254 | UUUGCUAUGCAAA | 634 | GCATTACAAATACCAT | 832 |
| | UGGCUUA | | TTGCTATGCAAATGGC | |
| | | | TTATAGGTTTAAT | |
| S_22276 | UCAAACUUUACUU | 635 | TAACATCACTAGGTTT | 833 |
| | GCUUUAC | | CAAACTTTACTTGCTTT | |
| | | | ACATAGAAGTTA | |
| S_23270 | GCUGACACUACUGA | 636 | TTTGGCAGAGACATTG | 834 |
| | UGCUGU | | CTGACACTACTGATGC | |
| | | | TGTCCGTGATCCA | |
| S_25235 | AUUGCCAUAGUAA | 637 | TTTATAGCTGGCTTGAT | 835 |
| | UGGUGAC | | TGCCATAGTAATGGTG | |
| | | | ACAATTATGCTT | |
| S_23842 | UACACAAUUAAACC | 638 | ATATGGCAGTTTTTGT | 836 |
| | GUGCUU | | ACACAATTAAACCGTG | |
| | | | CTTTAACTGGAAT | |
| S_23307 | UUGAGAUUCUUGA | 639 | GTGATCCACAGACACT | 837 |
| | CAUUACA | | TGAGATTCTTGACATT | |
| | | | ACACCATGTTCTT | |
| S_21808 | UGUCCUACCAUUUA | 640 | GAGGTTTGATAACCCT | 838 |
| | AUGAUG | | GTCCTACCATTTAATG | |
| | | | ATGGTGTTTATTT | |
| S_23935 | ACCACCAAUUAAAG | 641 | ACAAATTTACAAAACA | 839 |
| | AUUUUG | | CCACCAATTAAAGATT | |
| | | | TTGGTGGTTTTAA | |

TABLE 8
Top SARS-CoV-2 based on homology.

| | | SEQ | | SEQ |
|---|---|---|---|---|
| | | ID | | ID |
| Sequence ID | 20 nt Sequence | NO: | 45 nt Gene Region | NO: |
| E_26313 | CGUGGUAUUCU | 64 | TCTTTTTCTTGCTTTCGTGGTATT | 172 |
| | UGCUAGUUA | | CTTGCTAGTTACACTAGCCAT | |
| 8b_27986 | UGUAGUUGAUG | 97 | TCAACATCAACCATATGTAGTTG | 205 |
| | ACCCGUGUC | | ATGACCCGTGTCCTATTCACTT | |
| 7a_27720 | UAUAACACUUU | 93 | TGCGGCAATAGTGTTTATAACAC | 201 |
| | GCUUCACAC | | TTTGCTTCACACTCAAAAGAAA | |
| orf1ab_14080 | GGUAACUGGUA | 11 | AATCAAGATCTCAATGGTAACTG | 119 |
| | UGAUUUCGG | | GTATGATTTCGGTGATTTCATA | |
| M_26581 | GGAACCUAGUA | 72 | AGCTCCTTGAACAATGGAACCTA | 180 |
| | AUAGGUUUC | | GTAATAGGTTTCCTATTCCTTA | |
| S_22820 | GAUUAUAAUUA | 38 | ACTGGAAAGATTGCTGATTATAA | 146 |
| | UAAAUUACC | | TTATAAATTACCAGATGATTTT | |
| orf1ab_21391 | UUUGACAUGAG | 22 | TCTTCCTATTCTTTATTTGACATG | 130 |
| | UAAAUUUCC | | AGTAAATTTCCCCTTAAATTA | |
| orf1ab_17107 | UUUGCUAUUGG | 16 | ACTGGTAAGAGTCATTTTGCTAT | 124 |
| | CCUAGCUCU | | TGGCCTAGCTCTCTACTACCCT | |
| orf1ab_20892 | UGCACCAGGUA | 21 | TTCTGATAAAGGAGTTGCACCAG | 129 |
| | CAGCUGUUU | | GTACAGCTGTTTTAAGACAGTG | |
| orf1ab_20497 | UCUGUUAUUGA | 20 | TCTAAGTGTGTGTGTTCTGTTATT | 128 |
| | UUUAUUACU | | GATTTATTACTTGATGATTTT | |
| N_28655 | GACGGCAUCAU | 108 | TATGGTGCTAACAAAGACGGCAT | 216 |
| | AUGGGUUGC | | CATATGGGTTGCAACTGAGGGA | |
| S_24289 | UGGAGUUACAC | 45 | TAGGTTTAATGGTATTGGAGTTA | 153 |
| | AGAAUGUUC | | CACAGAATGTTCTCTATGAGAA | |
| M_26624 | UCUACAAUUUG | 74 | TACATGGATTTGTCTTCTACAATT | 182 |
| | CCUAUGCCA | | TGCCTATGCCAACAGGAATAG | |
| orf1a_7643 | GCUGGUAGUAC | 28 | TGTGATACATTCTGTGCTGGTAG | 136 |
| | AUUUAUUAG | | TACATTTATTAGTGATGAAGTT | |
| orf1a_12932 | CCUAAAGUGAA | 34 | GACACACCTAAAGGTCCTAAAGT | 142 |
| | GUAUUUAUA | | GAAGTATTTATACTTTATTAAA | |
| E_26305 | CUUGCUUUCGU | 63 | AGCGTACTTCTTTTTCTTGCTTTC | 171 |
| | GGUAUUCUU | | GTGGTATTCTTGCTAGTTACA | |
| M_26602 | UAUUCCUUACA | 73 | TAGTAATAGGTTTCCTATTCCTTA | 181 |
| | UGGAUUUGU | | CATGGATTTGTCTTCTACAAT | |
| 3a_25630 | GUUUGCAACUU | 48 | AAGGGTGTTCACTTTGTTTGCAA | 156 |
| | GCUGUUGUU | | CTTGCTGTTGTTGTTTGTAACA | |
| E_26455 | CCUGAUCUUCU | 67 | AATTCTTCTAGAGTTCCTGATCTT | 175 |
| | GGUCUAAAC | | CTGGTCTAAACGAACTAAATA | |
| E_26463 | UCUGGUCUAAA | 68 | TAGAGTTCCTGATCTTCTGGTCTA | 176 |
| | CGAACUAAA | | AACGAACTAAATATTATATTA | |
| S_22550 | CCUAAUAUUAC | 37 | TCTATTGTTAGATTTCCTAATATT | 145 |
| | AAACUUGUG | | ACAAACTTGTGCCCTTTTGGT | |
| orf1a_8744 | UUUGCUAACAA | 31 | TCTACAGATACTTGTTTTGCTAAC | 139 |
| | ACAUGCUGA | | AAACATGCTGATTTTGACACA | |
| orf1ab_17370 | GGCCACAAAUU | 17 | TGATGAAATTTCAATGGCCACAA | 125 |
| | AUGAUUUGA | | ATTATGATTTGAGTGTTGTCAA | |
| 7a_27633 | AUCAGUUUCAC | 88 | TCAGTTACGTGCCAGATCAGTTT | 196 |
| | CUAAACUGU | | CACCTAAACTGTTCATCAGACA | |
| M_27014 | GCCUAAAGAAA | 79 | TGACATCAAGGACCTGCCTAAAG | 187 |
| | UCACUGUUG | | AAATCACTGTTGCTACATCACG | |
| S_25375 | UUACACAUAAA | 46 | AGGAGTCAAATTACATTACACAT | 154 |
| | CGAACUUAU | | AAACGAACTTATGGATTTGTTT | |
| 7a_27715 | GUGUUUAUAAC | 92 | ATTGTTGCGGCAATAGTGTTTAT | 200 |
| | ACUUUGCUU | | AACACTTTGCTTCACACTCAAA | |

TABLE 9
Antisense oligonucleotides targeting viral factors

| | | SEQ ID |
|---|---|---|
| Oligo ID | Sequence | NO: |
| orf1a_416 | ACTTCTACTAAGCCAC | 840 |
| orf1a_2290 | AAGCTTAAAGAATGTC | 841 |
| orf1a_8744-1 | CAGCATGTTTGTTAGC | 842 |
| orf1a_8744-2 | GCATGTTTGTTAGCAA | 843 |
| orf1a_9679-1 | AAGCAATTGTTATCCA | 844 |
| orf1a_9679-2 | TAAGCAATTGTTATCC | 845 |
| orf1ab_14361 | GTTTGCACAATGCAGA | 846 |
| orf1ab_17107 | AGAGCTAGGCCAATAG | 847 |
| orf1ab_18025 | AGCTTGTAAAGTTGCC | 848 |
| orf1ab_20892-1 | CAGCTGTACCTGGTGC | 849 |
| orf1ab_20892-2 | ACAGCTGTACCTGGTG | 850 |
| orf1ab_20892-3 | AACAGCTGTACCTGGT | 851 |
| S_22223-1 | AATGGTTCTAAAGCCG | 852 |
| S_22223-2 | CAATGGTTCTAAAGCC | 853 |
| S_23174 | TGAAGTTGAAATTGAC | 854 |
| S_23774 | CCACAAATGTACATTG | 855 |
| S_23778 | CAATGTACATTTGTGG | 856 |
| S_25375-1 | AGTTCGTTTATGTGTA | 857 |
| S_25375-2 | TAAGTTCGTTTATGTG | 858 |
| 3a_25717 | GAAGTAGACTAAAGCA | 859 |
| 3a_25914 | AATAGGACTTGTTGTG | 860 |
| 3a_25992 | AACTGTGTAATACAAC | 861 |
| 3a_26018-1 | ACAGCTGGTAATAGTC | 862 |
| 3a_26018-2 | CAGCTGGTAATAGTCT | 863 |
| 3a_26018-3 | TACAGCTGGTAATAGT | 864 |
| E_26258 | TACCTGTCTCTTCCGA | 865 |
| E_26261 | TAACGTACCTGTCTCT | 866 |
| E_26369-1 | AACAATATTGCAGCAG | 867 |
| E_26369-2 | TAACAATATTGCAGCA | 868 |
| E_26374-1 | CGTTAACAATATTGCA | 869 |
| E_26374-2 | CTCACGTTAACAATAT | 870 |
| M_26581-1 | ACCTATTACTAGGTTC | 871 |
| M_26581-2 | AAACCTATTACTAGGT | 872 |
| M_26602-1 | ACAAATCCATGTAAGG | 873 |
| M_26602-2 | CAAATCCATGTAAGGA | 874 |
| M_26624 | TGGCATAGGCAAATTG | 875 |
| M_26717 | GTAAACAGCAGCAAGC | 876 |
| 7a_27455 | GTACCTCTAACACACT | 877 |
| 7a_27553 | AGCAAGTCAGTGCAAA | 878 |
| 7a_27565-1 | ATTGAGTGCTAAAGCA | 879 |
| 7a_27565-2 | GCAAATTGAGTGCTAA | 880 |
| 7a_27705 | AAACACTATTGCCGCA | 881 |
| 7a_27720 | GTGTGAAGCAAAGTGT | 882 |
| 8b_27940 | AAACTACATTCTTGGT | 883 |
| 8b_28002 | AGAAGTGAATAGGACA | 884 |
| 8b_28091-1 | TGAATGGGTGATTTAG | 885 |
| 8b_28091-2 | GAATGGGTGATTTAGA | 886 |
| 8b_28119 | AAACTGTATAATTACC | 887 |
| 8b_28163 | CCCAATTTAGGTTCCT | 888 |
| N_28655 | ACCCATATGATGCCGT | 889 |
| N_28945-1 | CAAGCTGGTTCAATCT | 890 |
| N_28945-2 | GCTGGTTCAATCTGTC | 891 |
| N_29141-1 | CCTTGTCTGATTAGTT | 892 |
| N_29141-2 | CTTGTCTGATTAGTTC | 893 |
| N_29307 | AATGACTTGATCTTTG | 894 |

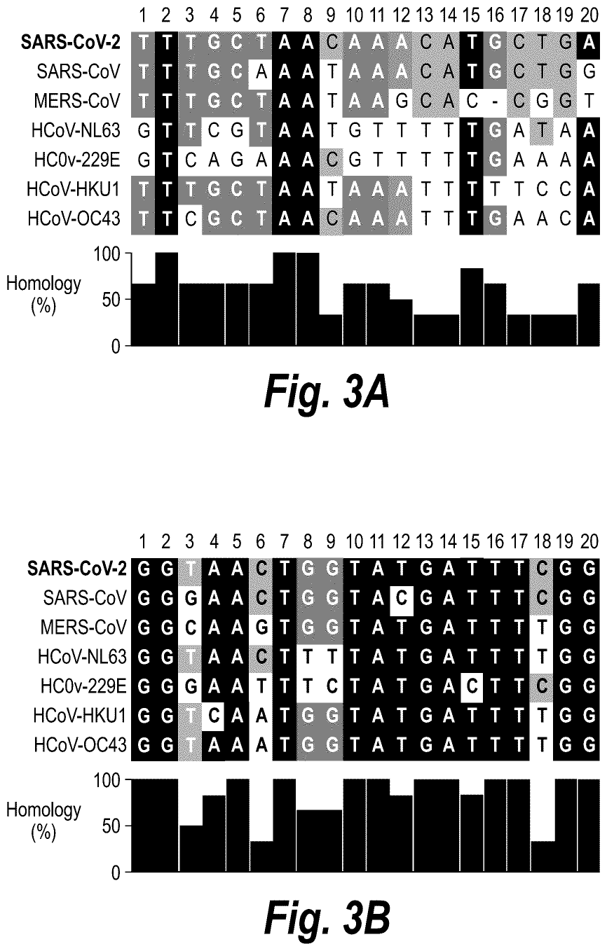

An alignment of siRNA and ASO selected for synthesis to six closely-related CoVs using the novel algorithm is shown in FIG. 3. Aligned genome regions of CoVs are shaded based on homology with darker coloring indicating higher homology with respect to SARS-CoV-2. The siRNA position is indicated on the top. Per position percent homology of SARS-CoV-2 to the six related CoVs is plotted on the bottom. SiRNA with low homology scores of 59 are shown in FIG. 3A. SiRNA with a high homology scores of score of 78 are shown in FIG. 3B. Gaps in alignment are indicated with dashes (-).

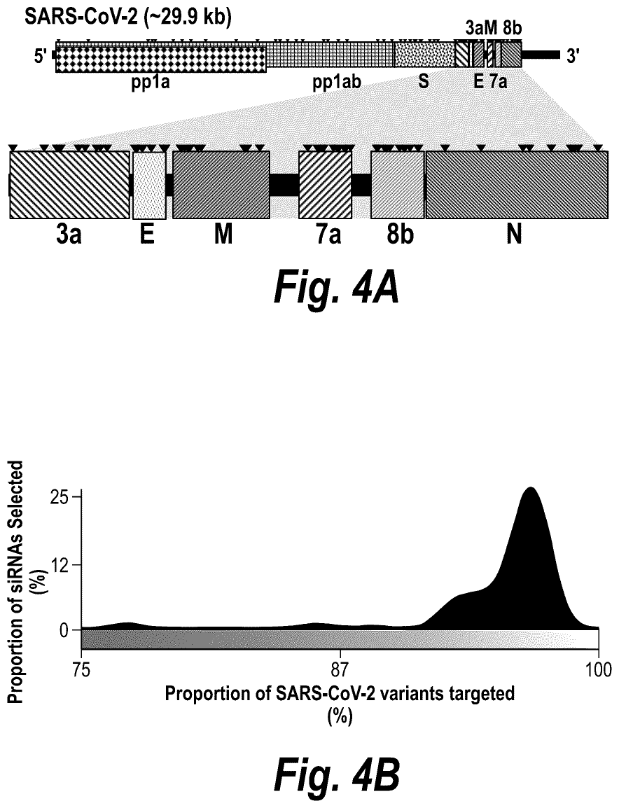

SiRNA and ASO target selections were based on the ability to target many SARS-CoV-2 genomes from patient isolates. siRNAs and ASOs were selected to target regions of the 9 selection genes with low mutation rates in other coronaviruses (FIG. 4A). The proportion of SARS-CoV-2 variants from patient isolates targeted by all selected siRNAs is plotted at the bottom. siRNAs and ASOs were selected to target regions of the 9 selection genes with low mutation rates in other coronaviruses (see previous figure), which resulted in selecting siRNAs that together target all isolates from SARS-CoV-infected patients, with >90% of siRNAs and ASOs selected targeting >95% of these genomes. The proportion of SARS-CoV-2 variants from patient isolates targeted by all selected siRNAs is plotted at the bottom. The coloured gradient indicates proportion of variant genomes targeted, with red indicating siRNAs that target fewer variants (with the lowest value being 77% of all variants targeted) and white indicating higher number of genomes targeted. Arrows in the gene diagram indicate siRNA and ASO target positions in the SARS-CoV2 genome and are coloured based on proportion of SARS-CoV-2 variants targeted and correlate with the colored gradient in the bottom plot. Dark red arrows indicate siRNAs that target <85% of genomes from patient isolates, while white and light pink arrows indicate siRNAs that target >90% of genomes SARS-CoV-2 isolates patients.

The scoring scheme methodology resulted in the selection of siRNAs that target all 708 SARS-CoV2 patient isolates available at the time of design with >95% of siRNAs (103 siRNAs) selected targeting more than 90% of the SARS-CoV2 genomes obtained from patient isolates. Furthermore, >60% of siRNAs (66 siRNAs) selected target more than 97% of these patient isolates.

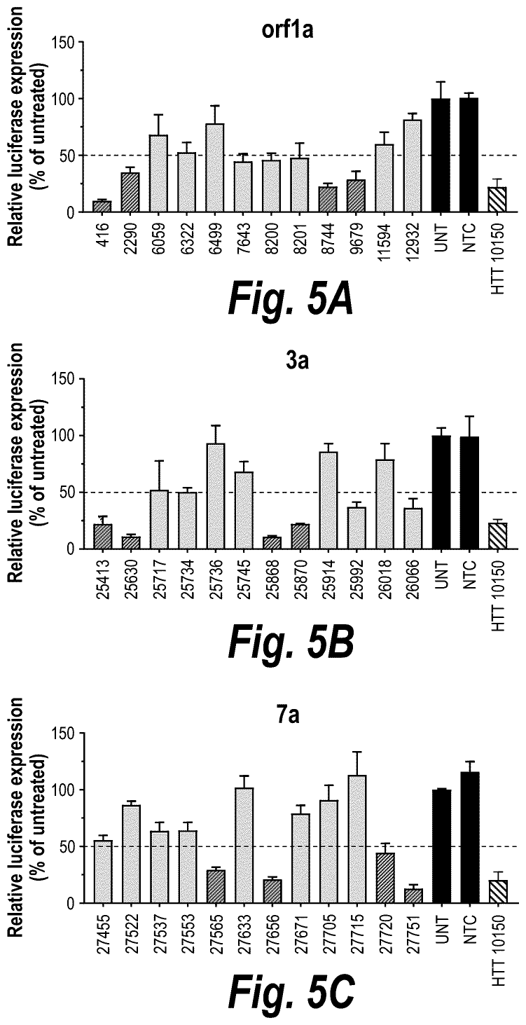

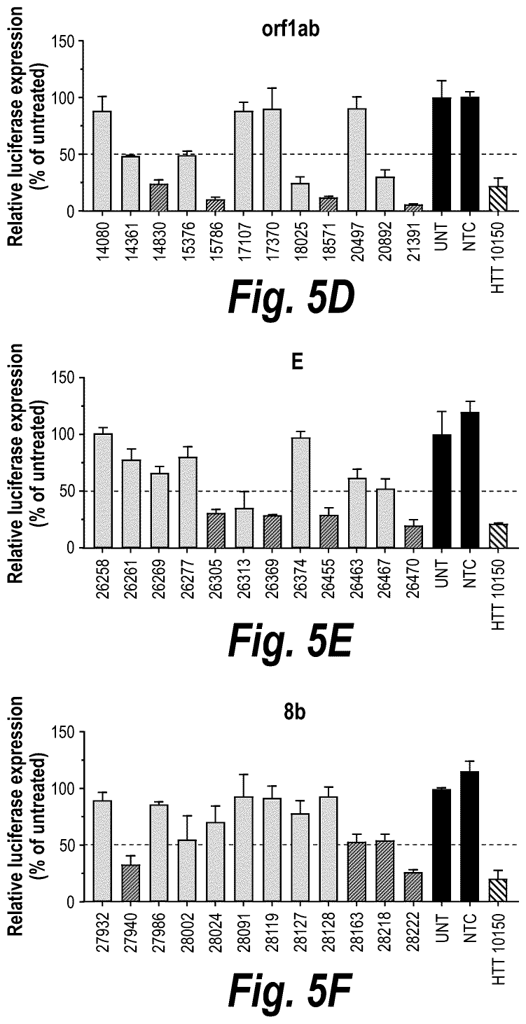

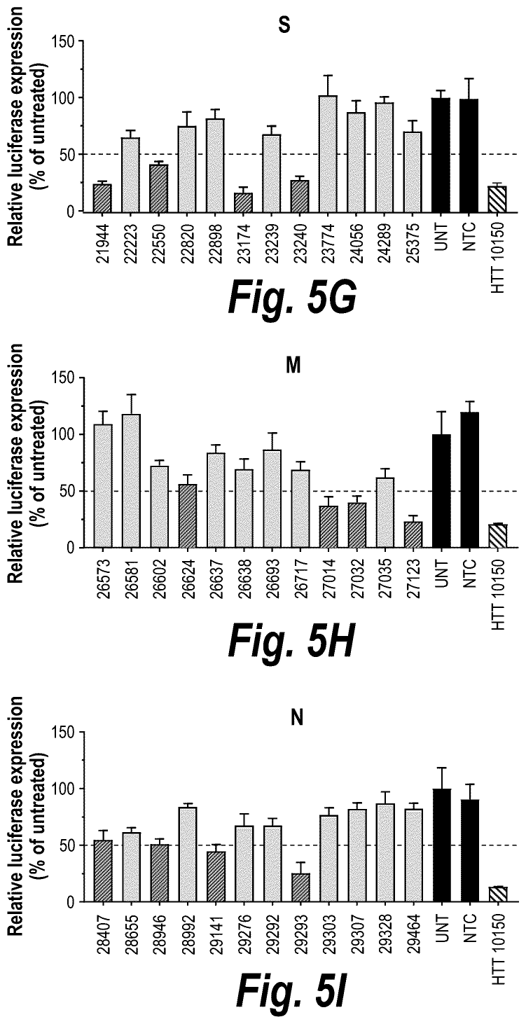

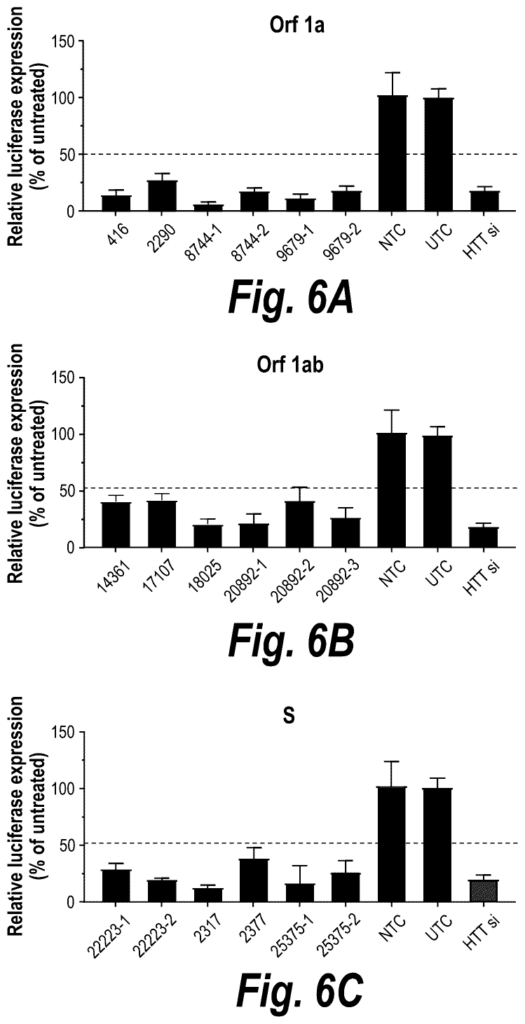

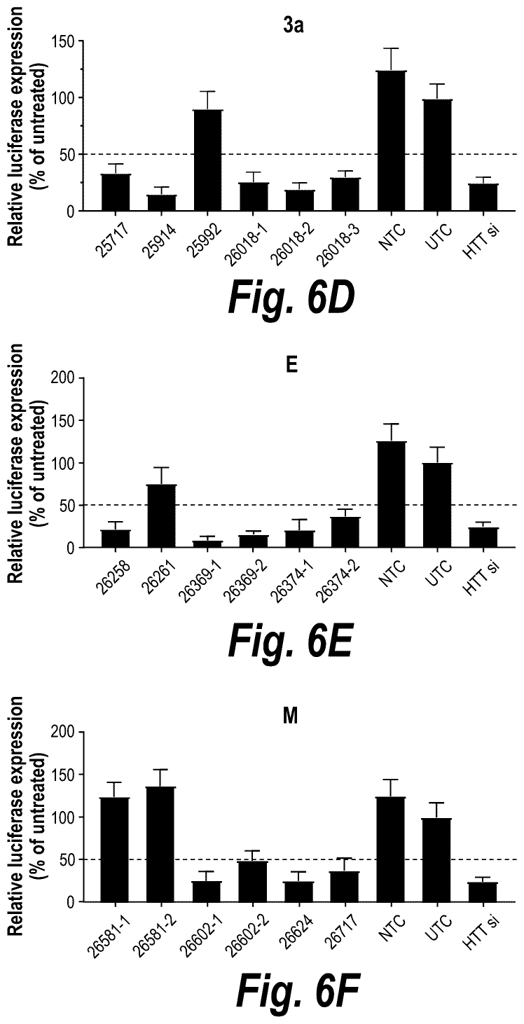

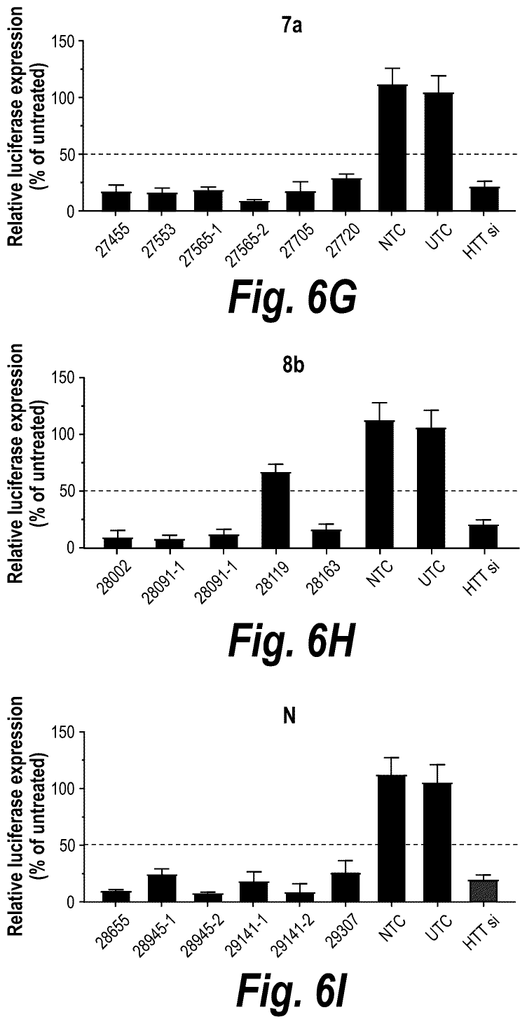

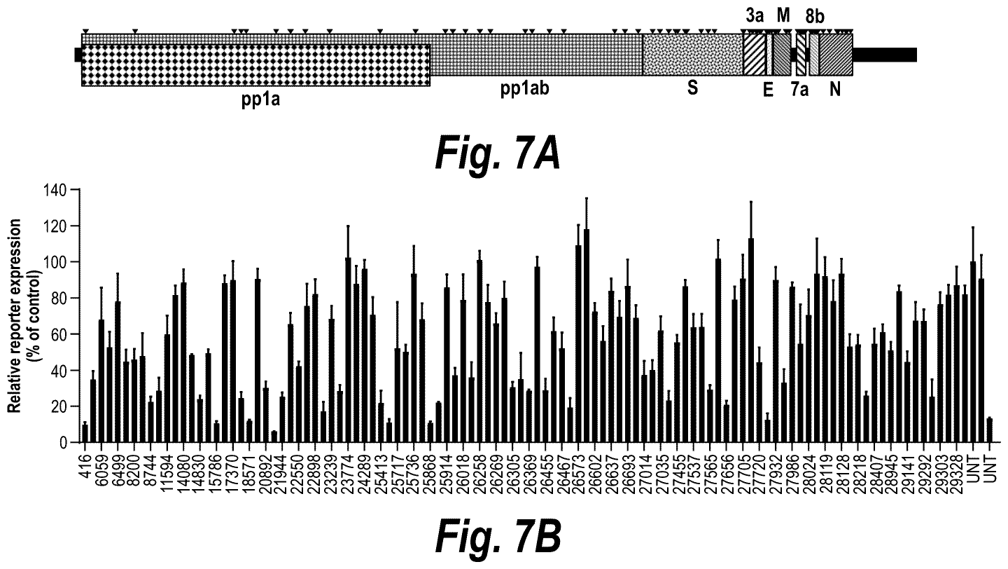

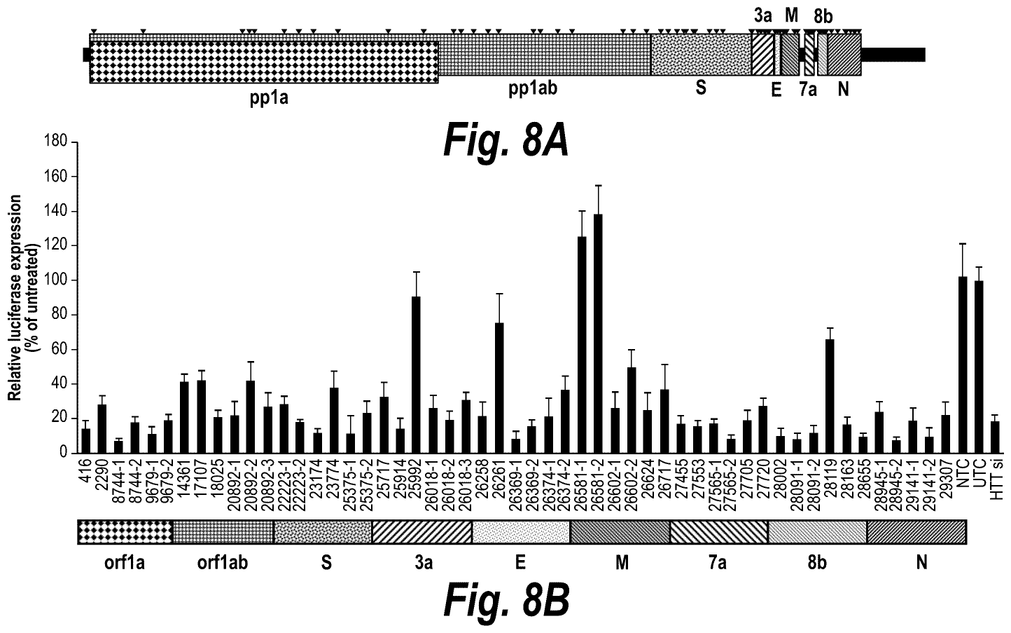

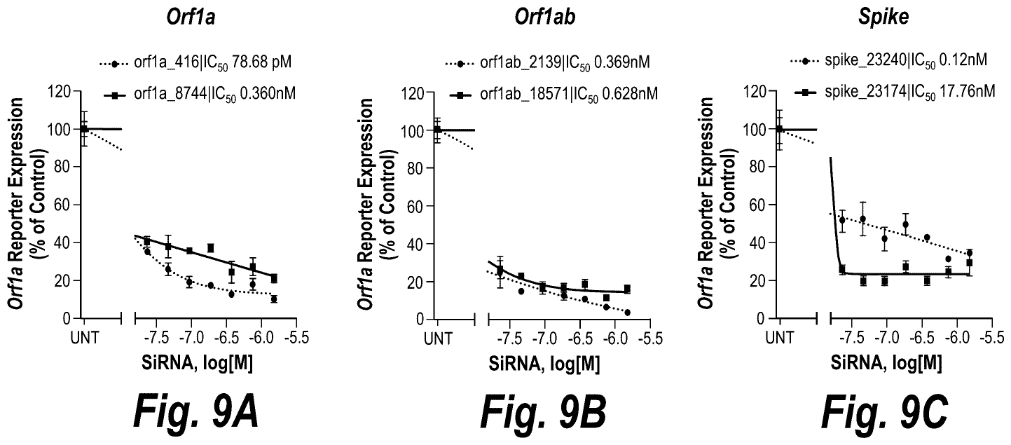

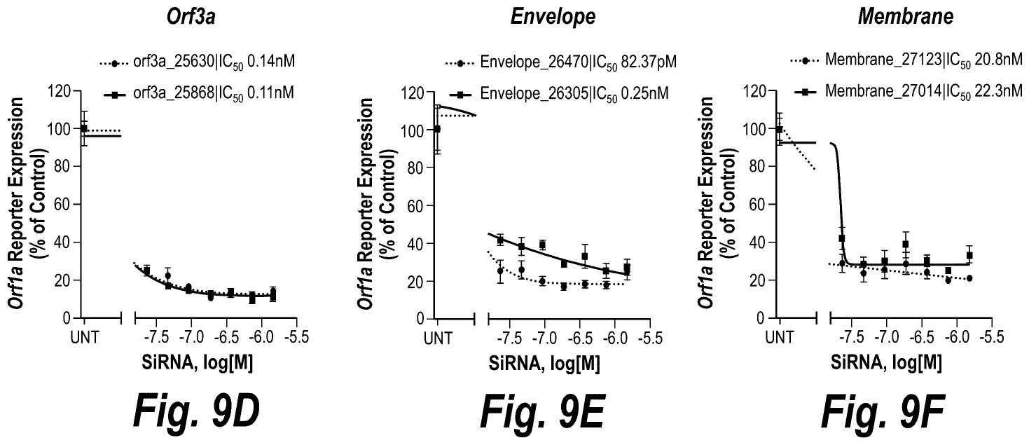

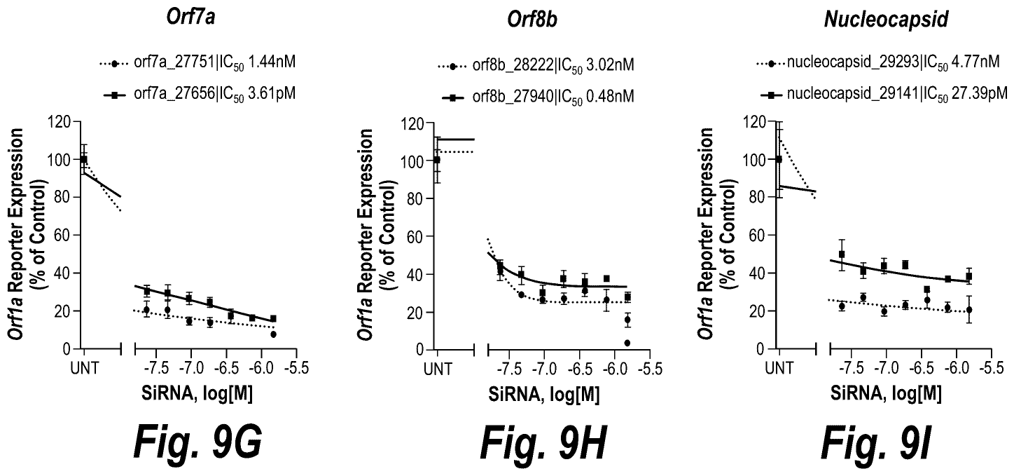

SiRNAs targeting different genes in the SARS-CoV2 genome were tested for silencing efficacy of the orf1a, 3a, 7a, orf1ab, E, gene 8b, S, M and N genes (FIG. 5, FIG. 7). For each target, at least 3 siRNAs were identified that that reduced target mRNA expression below 75% compared to untreated controls. siRNAs were tested in Hela cells and silencing was assessed using the psi-check reporter system, using an siRNA concentration of 1.5 uM and an assessment timepoint of 72 hours. Likewise, ASOs targeting the different genes in the SARS-CoV2 genome were tested for silencing efficacy, as depicted in FIG. 6 and FIG. 8. For each target, at least 3 ASOs were identified that reduced target mRNA expression below 75% compared to untreated controls. SiRNAs targeting the orf1a, 3a, 7a, orf1ab, E, gene 8b, S, M and N genes in the SARS-CoV2 genome were subsequently tested for silencing efficacy in 8-point dose response studies. Each siRNA showed potent and efficacious target silencing with IC₅₀ values in the low nanomolar range.

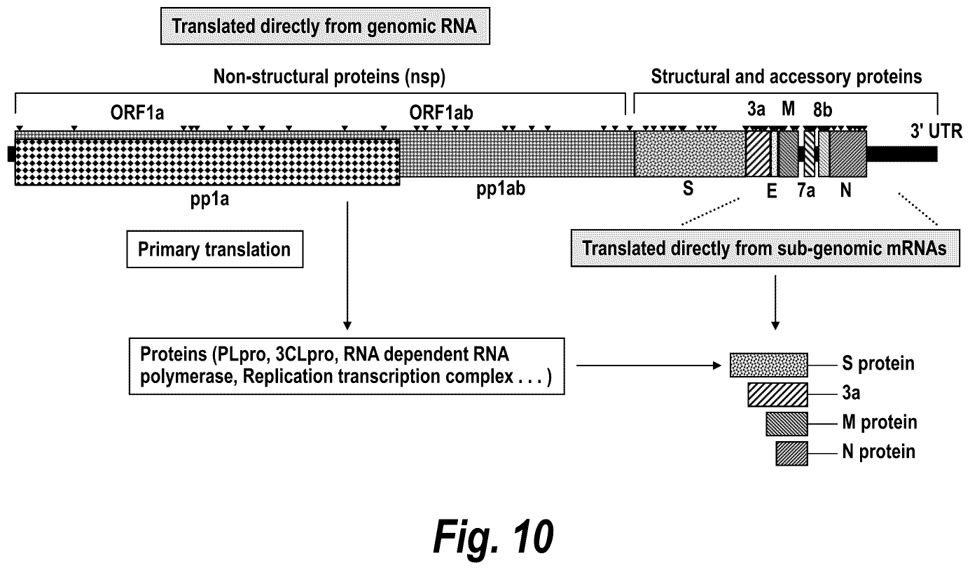

FIG. 10 depicts a schematic showing genes comprising genome and their functions. Comprised of structural and non-structural genes. Non-structural genes undergo primary translation while structural and accessory proteins are translated from sub-genomic mRNAs. The secondary structure of sub-genomic mRNAs may enhance targetability by siRNAs.

Development and optimization of siRNA cocktails targeting SARS-CoV2. Like other RNA viruses, SARS-CoV2 mutates. Therefore, multiple siRNAs in cocktails are necessary to minimize the chances of mutant development. Several siRNA cocktails were developed targeting g+strand only, g+ strand and terminal 3′UTR (region shared by all secondary mRNA variants), as well as a variety of other combinations. The cocktails are screened in SARS-CoV-2 infected VERO6 cells to define optimal siRNA combinations against the live virus. The strategy of targeting the + strand (orf1a and orf1ab with S and N protein) is particularly novel and active in blocking SARS-CoV-2 infection. The cocktails are combinations of at least 3 siRNAs targeting different regions of the viral genome. Table 10 shows the compositions of the various cocktails tested.

TABLE 10
List of siRNA combinations for targeting SARS-COV-2

| Combo | Code name | Strategy | Compounds (Sequence IDs) | | |
|---|---|---|---|---|---|
| 1 | Cosmopolitan | Replication | 416 | 9679 | 21391 |
| 2 | Screwdriver | Replication | 416 | 8744 | 21391 |
| 3 | Long Island | Replication | 9679 | 8744 | 21391 |
| 4 | Negroni | Rep/Immun | 416 | 9679 | 27565 |
| 5 | Old Fashioned | Rep/Immun | 416 | 21391 | 27565 |
| 6 | Manhattan | Rep/Immun | 21391 | 27656 | 27751 |
| 7 | Moscow Mule | Rep/capsid | 416 | 23174 | 26305 |
| 8 | Daiquiri | Rep/capsid | 9679 | 23174 | 29293 |
| 9 | Martini | Rep/capsid | 21391 | 26305 | 29293 |
| 10 | Jaegger Bomb | Immun/capsid | 23174 | 26470 | 27565 |
| 11 | Bloody Mary | Immun/capsid | 23174 | 27123 | 27656 |
| 12 | White Russian | Immun/capsid | 23174 | 27032 | 27751 |
| 13 | Mojito | Rep/imm/cap | 416 | 26305 | 27565 |
| 14 | Salty Dog | Rep/imm/cap | 9679 | 26369 | 27656 |
| 15 | Hanky Panky | Rep/imm/cap | 21391 | 27032 | 27751 |

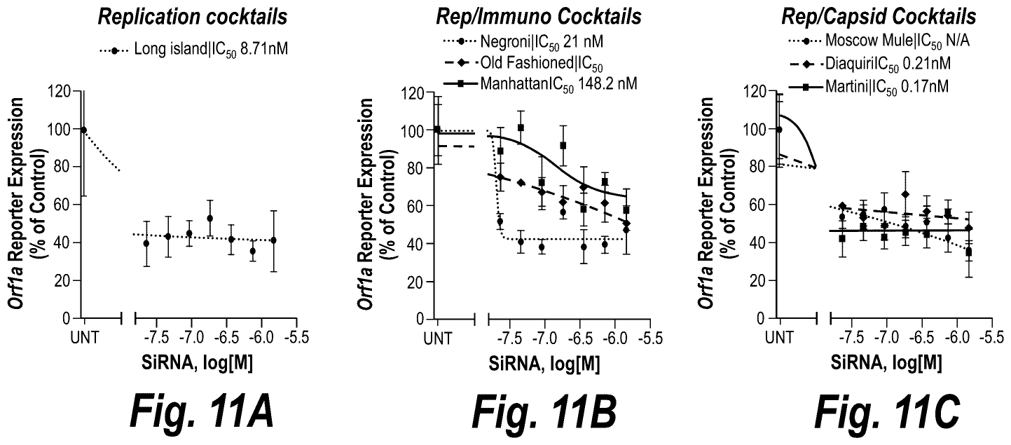

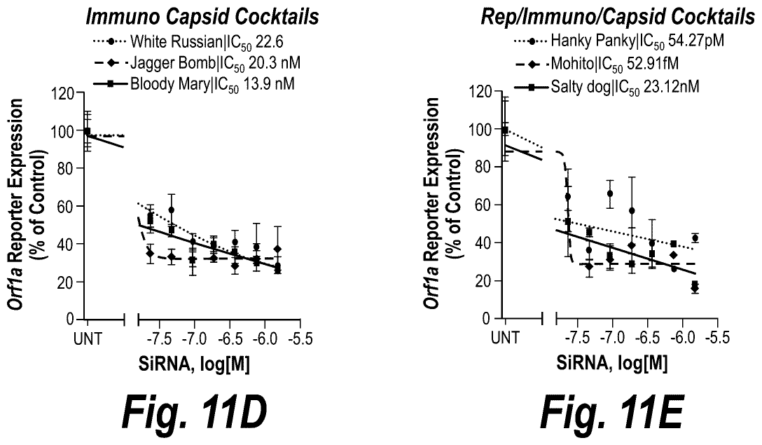

The validation and determination of IC₅₀ values for siRNA cocktails targeting SARS-CoV-2 genes are depicted in FIG. 11. SiRNA cocktails targeting different genes in the SARS-CoV2 genome were tested for silencing efficacy in 8-point dose response studies. Each siRNA showed potent and efficacious target silencing with IC₅₀ values in the low nanomolar range. Results for replication cocktails are shown in FIG. 11A, for Replication/Immuno cocktails in FIG. 11B, for Replication/Capsid cocktails in FIG. 11C, for Immuno/Capsid cocktails in FIG. 11D, and for Replication/Immuno/Capsid cocktails in FIG. 11E.

### Example 2. Targeting of Human Genes

SiRNAs targeting of ACE2, FURIN, TMPRSS2, IL-6, and IL-6 Receptor (IL-6R) can be used alone or in combination with siRNAs targeting SARS-CoV2 for comprehensive treatment of SARS-CoV-2 treatment. Hyper functional, fully chemical stabilized siRNAs were identified targeting these a selection of these host cell genes involved in infection and spread.

Studies of ACE2 knock out mice show some toxicity in the heart and muscle, thus limiting the use of traditional small molecules and antibodies that do not differentiate between tissues. EPA-conjugates have no functional delivery to muscle and heart, and thus might be a better option for ACE2 modulation. In addition, local intratracheal delivery of di-valent compounds results in minimal heart and muscle delivery, representing a very powerful option for modulation of host genes, where lung-selective targeting with minimized overall exposure is required.

Host target 45 nucleotide gene regions and 20 nucleotide target regions are summarized in Table 11A-11D and Table 12A-12E.

TABLE 11A
Host target TMPRSS-20 nucleotide targets and 45 nucleotide gene target regions

| SEQUENCE | | SEQ ID | | SEQ ID | |
|---|---|---|---|---|---|
| ID | 20 nt Sequence | NO: | 45 nt Gene Region | NO: | Species |
| TMPRSS | UCCCGCAU | 895 | AGTGACTCATGTTCAT | 1095 | mouse |
| 2_924 | GGUGGUUU | | CCCGCATGGTGGTTTC | | |
| | CUUU | | TTTGCGCTGTATA | | |
| TMPRSS | UACCACAG | 896 | CTCTATAAAAAACTCT | 1096 | mouse |
| 2_903 | UGACUCAU | | ACCACAGTGACTCATG | | |
| | GUUC | | TTCATCCCGCATG | | |
| TMPRSS | UGAGCUCA | 897 | TTATGAAGCTGAATGT | 1097 | mouse |
| 2_868 | GGCAACGU | | GAGCTCAGGCAACGT | | |
| | UGAC | | TGACCTCTATAAAA | | |
| TMPRSS | UCUUCAAA | 898 | CACAGTGATGCCTGTT | 1098 | human |
| 2_866 | AGCAGUGG | | CTTCAAAAGCAGTGGT | | |
| | UUUC | | TTCTTTACGCTGT | | |
| TMPRSS | UGUGAGCU | 899 | CTTTATGAAGCTGAAT | 1099 | mouse |
| 2_866 | CAGGCAAC | | GTGAGCTCAGGCAAC | | |
| | GUUG | | GTTGACCTCTATAA | | |
| TMPRSS | ACCACAGU | 900 | TCTATAAAAAACTGTA | 1100 | human |
| 2_849 | GAUGCCUG | | CCACAGTGATGCCTGT | | |
| | UUCU | | TCTTCAAAAGCAG | | |
| TMPRSS | CCGGCAAU | 901 | AACTGAACACAAGTG | 1101 | human |
| 2_819 | GUCGAUAU | | CCGGCAATGTCGATAT | | |
| | CUAU | | CTATAAAAAACTGT | | |
| TMPRSS | AGUGCCGG | 902 | ATGAAACTGAACACA | 1102 | human |
| 2_815 | CAAUGUCG | | AGTGCCGGCAATGTC | | |
| | AUAU | | GATATCTATAAAAAA | | |
| TMPRSS | AAAGACAU | 903 | GGGAGAGCAGCATGT | 1103 | mouse |
| 2_783 | GGGAUACA | | AAAGACATGGGATAC | | |
| | AGAA | | AAGAACAATTTTTAT | | |
| TMPRSS | UUUACUCU | 904 | GCTATAAGAATAATTT | 1104 | human |
| 2_753 | AGCCAAGG | | TTACTCTAGCCAAGGA | | |
| | AAUA | | ATAGTGGATGACA | | |
| TMPRSS | GCUUCAUC | 905 | GTCTCTACGGACAAA | 1105 | mouse |
| 2_691 | CUCCAGGU | | GCTTCATCCTCCAGGT | | |
| | UUAC | | TTACTCATCTCAGA | | |
| TMPRSS | ACGGACAA | 906 | GTTGTGTTCGTCTCTA | 1106 | mouse |
| 2_682 | AGCUUCAU | | CGGACAAAGCTTCATC | | |
| | CCUC | | CTCCAGGTTTACT | | |
| TMPRSS | UGGGUCUU | 907 | GTCTGAGATGGAGTGT | 1107 | mouse |
| 2_587 | CAGGCACA | | GGGTCTTCAGGCACAT | | |
| | UGCA | | GCATCAGCTCTTC | | |
| TMPRSS | UCUGGGAC | 908 | TCTTGCTTTGGAGGTT | 1108 | mouse |
| 2_550 | AGCAACUG | | CTGGGACAGCAACTG | | |
| | UUCU | | TTCTACGTCTGAGA | | |
| TMPRSS | ACCUGCAU | 909 | TGCGACTCCTCAGGTA | 1109 | human |
| 2_545 | CAACCCCU | | CCTGCATCAACCCCTC | | |
| | CUAA | | TAACTGGTGTGAT | | |
| TMPRSS | CUUGCUUU | 910 | TGCTGTGGCTGCTGTC | 1110 | mouse |
| 2_536 | GGAGGUUC | | TTGCTTTGGAGGTTCT | | |
| | UGGG | | GGGACAGCAACTG | | |
| TMPRSS | AUGGGCAG | 911 | CTACTCTGGAAGTTCA | 1111 | human |
| 2_497 | CAAGUGCU | | TGGGCAGCAAGTGCT | | |
| | CCAA | | CCAACTCTGGGATA | | |
| TMPRSS | UAAGAAAU | 912 | GTGCACCTCAAAGTCT | 1112 | mouse |
| 2_470 | CGCUGUGU | | AAGAAATCGCTGTGTT | | |
| | UUAG | | TAGCCCTCGCCCT | | |
| TMPRSS | CUCAAAGU | 913 | AGGAGCACTGTGCAC | 1113 | mouse |
| 2_461 | CUAAGAAA | | CTCAAAGTCTAAGAA | | |
| | UCGC | | ATCGCTGTGTTTAGC | | |
| TMPRSS | UAAGAAAG | 914 | GTGCACCTCAAAGACT | 1114 | human |
| 2_415 | CACUGUGC | | AAGAAAGCACTGTGC | | |
| | AUCA | | ATCACCTTGACCCT | | |
| TMPRSS | CUCAACAU | 915 | GATTACAACGCAAGC | 1115 | mouse |
| 2_410 | CUGUCAUC | | CTCAACATCTGTCATC | | |
| | CACA | | CACACACATCCCAA | | |
| TMPRSS | AUCAUGCA | 916 | ATTGTGAAAATGAAT | 1116 | human |
| 2_3204 | AAUAAAUU | | ATCATGCAAATAAATT | | |
| | AUGC | | ATGCAATTTTTTTT | | |
| TMPRSS | UAUCAUGC | 917 | AATTGTGAAAATGAA | 1117 | human |
| 2_3203 | AAAUAAAU | | TATCATGCAAATAAAT | | |
| | UAUG | | TATGCAATTTTTTT | | |
| TMPRSS | GUGAAAAU | 918 | CTGTAAAGTTCAATTG | 1118 | human |
| 2_3192 | GAAUAUCA | | TGAAAATGAATATCAT | | |
| | UGCA | | GCAAATAAATTAT | | |
| TMPRSS | UUGUGAAA | 919 | AACTGTAAAGTTCAAT | 1119 | human |
| 2_3190 | AUGAAUAU | | TGTGAAAATGAATATC | | |
| | CAUG | | ATGCAAATAAATT | | |
| TMPRSS | UUCAAUUG | 920 | TTTTAAACTGTAAAGT | 1120 | human |
| 2_3185 | UGAAAAUG | | TCAATTGTGAAAATGA | | |
| | AAUA | | ATATCATGCAAAT | | |
| TMPRSS | GUUCAAUU | 921 | TTTTTAAACTGTAAAG | 1121 | human |
| 2_3184 | GUGAAAAU | | TTCAATTGTGAAAATG | | |
| | GAAU | | AATATCATGCAAA | | |
| TMPRSS | UGUAAAGU | 922 | GTATCTTTTTTAAACT | 1122 | human |
| 2_3178 | UCAAUUGU | | GTAAAGTTCAATTGTG | | |
| | GAAA | | AAAATGAATATCA | | |
| TMPRSS | AACUGUAA | 923 | TTTGTATCTTTTTTAA | 1123 | human |
| 2_3175 | AGUUCAAU | | ACTGTAAAGTTCAATT | | |
| | UGUG | | GTGAAAATGAATA | | |
| TMPRSS | UUAAACUG | 924 | TTTTTTGTATCTTTTTT | 1124 | human |
| 2_3172 | UAAAGUUC | | AAACTGTAAAGTTCA | | |
| | AAUU | | ATTGTGAAAATGA | | |
| TMPRSS | UUUAAACU | 925 | TTTTTTTGTATCTTTTT | 1125 | human |
| 2_3171 | GUAAAGUU | | TAAACTGTAAAGTTCA | | |
| | CAAU | | ATTGTGAAAATG | | |
| TMPRSS | GUGAACAA | 926 | CCCCTTCTTATTTATG | 1126 | human |
| 2_3139 | CUGUUUGU | | TGAACAACTGTTTGTC | | |
| | CUUU | | TTTTTTTGTATCT | | |
| TMPRSS | AUGUGAAC | 927 | TGCCCCTTCTTATTTA | 1127 | human |
| 2_3137 | AACUGUUU | | TGTGAACAACTGTTTG | | |
| | GUCU | | TCTTTTTTTGTAT | | |
| TMPRSS | UAUGUGAA | 928 | TTGCCCCTTCTTATTT | 1128 | human |
| 2_3136 | CAACUGUU | | ATGTGAACAACTGTTT | | |
| | UGUC | | GTCTTTTTTTGTA | | |
| TMPRSS | UUAUGUGA | 929 | ATTGCCCCTTCTTATT | 1129 | human |
| 2_3135 | ACAACUGU | | TATGTGAACAACTGTT | | |
| | UUGU | | TGTCTTTTTTTGT | | |
| TMPRSS | AUUUAUGU | 930 | TTATTGCCCCTTCTTA | 1130 | human |
| 2_3133 | GAACAACU | | TTTATGTGAACAACTG | | |
| | GUUU | | TTTGTCTTTTTTT | | |
| TMPRSS | AAAUAUAU | 931 | TGAGATTCCACTGTGA | 1131 | mouse |
| 2_3130 | GAAUAAAG | | AATATATGAATAAAG | | |
| | UAUA | | TATATAATTCTTTT | | |
| TMPRSS | UCUUAUUU | 932 | TTCTTTATTGCCCCTTC | 1132 | human |
| 2_3129 | AUGUGAAC | | TTATTTATGTGAACAA | | |
| | AACU | | CTGTTTGTCTTT | | |
| TMPRSS | AUUGCCCC | 933 | ACGTCTTCCTTCTTTA | 1133 | human |
| 2_3120 | UUCUUAUU | | TTGCCCCTTCTTATTT | | |
| | UAUG | | ATGTGAACAACTG | | |
| TMPRSS | GAUUCCAC | 934 | TGTTCTGAGCTGTGAG | 1134 | mouse |
| 2_3118 | UGUGAAAU | | ATTCCACTGTGAAATA | | |
| | AUAU | | TATGAATAAAGTA | | |
| TMPRSS | UCUGAGCU | 935 | CTGCTTTGTGTCTGTT | 1135 | mouse |
| 2_3106 | GUGAGAUU | | CTGAGCTGTGAGATTC | | |
| | CCAC | | CACTGTGAAATAT | | |
| TMPRSS | UGUGUCUG | 936 | GGTTATTTCCTGCTTT | 1136 | mouse |
| 2_3097 | UUCUGAGC | | GTGTCTGTTCTGAGCT | | |
| | UGUG | | GTGAGATTCCACT | | |
| TMPRSS | UAAUGGUG | 937 | TCCTAAAAGGTGTTGT | 1137 | human |
| 2_3094 | AAAACGUC | | AATGGTGAAAACGTC | | |
| | UUCC | | TTCCTTCTTTATTG | | |
| TMPRSS | GUAAUGGU | 938 | ATCCTAAAAGGTGTTG | 1138 | human |
| 2_3093 | GAAAACGU | | TAATGGTGAAAACGT | | |
| | CUUC | | CTTCCTTCTTTATT | | |
| TMPRSS | CCUGCUUU | 939 | GTTGGTTGGTTATTTC | 1139 | mouse |
| 2_3090 | GUGUCUGU | | CTGCTTTGTGTCTGTT | | |
| | UCUG | | CTGAGCTGTGAGA | | |
| TMPRSS | UUGGUUAU | 940 | TGTCTTTGCTGTTGGT | 1140 | mouse |
| 2_3080 | UUCCUGCU | | TGGTTATTTCCTGCTT | | |
| | UUGU | | TGTGTCTGTTCTG | | |
| TMPRSS | ACAUCCUA | 941 | CTTGCTCCCCAAGACA | 1141 | human |
| 2_3076 | AAAGGUGU | | CATCCTAAAAGGTGTT | | |
| | UGUA | | GTAATGGTGAAAA | | |
| TMPRSS | CUGUGCAC | 942 | ATTTGCAGGATCTGTC | 1142 | human |
| 2_2994 | AUGCCUCU | | TGTGCACATGCCTCTG | | |
| | GUAG | | TAGAGAGCAGCAT | | |
| TMPRSS | GCUGUAAG | 943 | GGGACCTTCTTAGATG | 1143 | mouse |
| 2_2988 | GUACCUAC | | CTGTAAGGTACCTACA | | |
| | AUAC | | TACAGACTAAATG | | |
| TMPRSS | UUUGCAGG | 944 | AGTCATGCAATCCCAT | 1144 | human |
| 2_2980 | AUCUGUCU | | TTGCAGGATCTGTCTG | | |
| | GUGC | | TGCACATGCCTCT | | |
| TMPRSS | AAUCCCAU | 945 | AATGGAAAGTCATGC | 1145 | human |
| 2_2973 | UUGCAGGA | | AATCCCATTTGCAGGA | | |
| | UCUG | | TCTGTCTGTGCACA | | |
| TMPRSS | GGGACCUU | 946 | CCTCATGCGTCCTCTG | 1146 | mouse |
| 2_2973 | CUUAGAUG | | GGACCTTCTTAGATGC | | |
| | CUGU | | TGTAAGGTACCTA | | |
| TMPRSS | UGGGACCU | 947 | CCCTCATGCGTCCTCT | 1147 | mouse |
| 2_2972 | UCUUAGAU | | GGGACCTTCTTAGATG | | |
| | GCUG | | CTGTAAGGTACCT | | |
| TMPRSS | UGGAAAGU | 948 | GACTTAACCTTGAAAT | 1148 | human |
| 2_2960 | CAUGCAAU | | GGAAAGTCATGCAAT | | |
| | CCCA | | CCCATTTGCAGGAT | | |
| TMPRSS | CCUUGAAA | 949 | ACAGCTAGGACTTAA | 1149 | human |
| 2_2952 | UGGAAAGU | | CCTTGAAATGGAAAG | | |
| | CAUG | | TCATGCAATCCCATT | | |
| TMPRSS | UACAGCUA | 950 | TGAAATGAATGATTCT | 1150 | human |
| 2_2936 | GGACUUAA | | ACAGCTAGGACTTAA | | |
| | CCUU | | CCTTGAAATGGAAA | | |
| TMPRSS | UUCUACAG | 951 | GAATGAAATGAATGA | 1151 | human |
| 2_2933 | CUAGGACU | | TTCTACAGCTAGGACT | | |
| | UAAC | | TAACCTTGAAATGG | | |
| TMPRSS | UGAAUGAU | 952 | TTTGCAAGAATGAAAT | 1152 | human |
| 2_2926 | UCUACAGC | | GAATGATTCTACAGCT | | |
| | UAGG | | AGGACTTAACCTT | | |
| TMPRSS | GUUUGCAA | 953 | TGGCCAAGCAGGCTG | 1153 | human |
| 2_2910 | GAAUGAAA | | GTTTGCAAGAATGAA | | |
| | UGAA | | ATGAATGATTCTACA | | |
| TMPRSS | CCAAGCAG | 954 | GGTTCTGCCTCCTGGC | 1154 | human |
| 2_2898 | GCUGGUUU | | CAAGCAGGCTGGTTTG | | |
| | GCAA | | CAAGAATGAAATG | | |
| TMPRSS | UGGGUCAU | 955 | TCAGAAGGCAGTGAA | 1155 | mouse |
| 2_2877 | AACUGGGA | | TGGGTCATAACTGGG | | |
| | CUCC | | ACTCCATCTTTGCTG | | |
| TMPRSS | ACCAAAUA | 956 | AGCCATGCCAGAATT | 1156 | mouse |
| 2_2796 | UGAAGUAU | | ACCAAATATGAAGTA | | |
| | GAAU | | TGAATGTCTTACCCA | | |
| TMPRSS | AUUACCAA | 957 | GAAAGCCATGCCAGA | 1157 | mouse |
| 2_2793 | AUAUGAAG | | ATTACCAAATATGAA | | |
| | UAUG | | GTATGAATGTCTTAC | | |
| TMPRSS | UGUGGUCC | 958 | TTGTTTTGGACTCTCT | 1158 | human |
| 2_2792 | CUUCCAAU | | GTGGTCCCTTCCAATG | | |
| | GCUG | | CTGTGGGTTTCCA | | |
| TMPRSS | GCCAGAAU | 959 | TACCAGGAAAGCCAT | 1159 | mouse |
| 2_2787 | UACCAAAU | | GCCAGAATTACCAAA | | |
| | AUGA | | TATGAAGTATGAATG | | |
| TMPRSS | UGCCAGAA | 960 | ATACCAGGAAAGCCA | 1160 | mouse |
| 2_2786 | UUACCAAA | | TGCCAGAATTACCAA | | |
| | UAUG | | ATATGAAGTATGAAT | | |
| TMPRSS | CAUGCCAG | 961 | TTATACCAGGAAAGC | 1161 | mouse |
| 2_2784 | AAUUACCA | | CATGCCAGAATTACCA | | |
| | AAUA | | AATATGAAGTATGA | | |
| TMPRSS | AAAGCCAU | 962 | TGGGTTTATACCAGGA | 1162 | mouse |
| 2_2779 | GCCAGAAU | | AAGCCATGCCAGAAT | | |
| | UACC | | TACCAAATATGAAG | | |
| TMPRSS | UAUACCAG | 963 | TTCTGGCCCTGGGTTT | 1163 | mouse |
| 2_2770 | GAAAGCCA | | ATACCAGGAAAGCCA | | |
| | UGCC | | TGCCAGAATTACCA | | |
| TMPRSS | GAAGAAGA | 964 | GCCCATGGTGGCGGC | 1164 | human |
| 2_2757 | GAAAGAUG | | GAAGAAGAGAAAGAT | | |
| | UGUU | | GTGTTTTGTTTTGGA | | |
| TMPRSS | CGGCGAAG | 965 | AAGTGCCCATGGTGG | 1165 | human |
| 2_2753 | AAGAGAAA | | CGGCGAAGAAGAGAA | | |
| | GAUG | | AGATGTGTTTTGTTT | | |
| TMPRSS | AGAGAGGA | 966 | TCAGATAAAGATGAA | 1166 | mouse |
| 2_2727 | GAUCAUUG | | AGAGAGGAGATCATT | | |
| | UCUU | | GTCTTCTGTCTTCTT | | |
| TMPRSS | UCAGAUAA | 967 | TAGAAGTTCCTAGCTT | 1167 | mouse |
| 2_2712 | AGAUGAAA | | CAGATAAAGATGAAA | | |
| | GAGA | | GAGAGGAGATCATT | | |
| TMPRSS | CCUAGCUU | 968 | ATGAGATTAGAAGTTC | 1168 | mouse |
| 2_2705 | CAGAUAAA | | CTAGCTTCAGATAAAG | | |
| | GAUG | | ATGAAAGAGAGGA | | |
| TMPRSS | CUCCUGAC | 969 | TGACAAAATGACTGG | 1169 | human |
| 2_2699 | UUAACGUU | | CTCCTGACTTAACGTT | | |
| | CUAU | | CTATAAATGAATGT | | |
| TMPRSS | GCUCCUGA | 970 | TTGACAAAATGACTG | 1170 | human |
| 2_2698 | CUUAACGU | | GCTCCTGACTTAACGT | | |
| | UCUA | | TCTATAAATGAATG | | |
| TMPRSS | GGCUCCUG | 971 | TTTGACAAAATGACTG | 1171 | human |
| 2_2697 | ACUUAACG | | GCTCCTGACTTAACGT | | |
| | UUCU | | TCTATAAATGAAT | | |
| TMPRSS | AUUAGAAG | 972 | TAGACTGGCAATGAG | 1172 | mouse |
| 2_2695 | UUCCUAGC | | ATTAGAAGTTCCTAGC | | |
| | UUCA | | TTCAGATAAAGATG | | |
| TMPRSS | AAUGAGAU | 973 | GTTCTGTAGACTGGCA | 1173 | mouse |
| 2_2689 | UAGAAGUU | | ATGAGATTAGAAGTTC | | |
| | CCUA | | CTAGCTTCAGATA | | |
| TMPRSS | GCAAUGAG | 974 | TTGTTCTGTAGACTGG | 1174 | mouse |
| 2_2687 | AUUAGAAG | | CAATGAGATTAGAAG | | |
| | UUCC | | TTCCTAGCTTCAGA | | |
| TMPRSS | UGACAAAA | 975 | CTATTTCAGCTGCTTT | 1175 | human |
| 2_2684 | UGACUGGC | | GACAAAATGACTGGC | | |
| | UCCU | | TCCTGACTTAACGT | | |
| TMPRSS | UUUGACAA | 976 | GCCTATTTCAGCTGCT | 1176 | human |
| 2_2682 | AAUGACUG | | TTGACAAAATGACTG | | |
| | GCUC | | GCTCCTGACTTAAC | | |
| TMPRSS | UAGGUUGU | 977 | CCTCTTCCAGATGGTT | 1177 | mouse |
| 2_2668 | UCUGUAGA | | AGGTTGTTCTGTAGAC | | |
| | CUGG | | TGGCAATGAGATT | | |
| TMPRSS | UUAGGUUG | 978 | ACCTCTTCCAGATGGT | 1178 | mouse |
| 2_2667 | UUCUGUAG | | TAGGTTGTTCTGTAGA | | |
| | ACUG | | CTGGCAATGAGAT | | |
| TMPRSS | UUCCAGAU | 979 | CAGACACTAGACCTCT | 1179 | mouse |
| 2_2657 | GGUUAGGU | | TCCAGATGGTTAGGTT | | |
| | UGUU | | GTTCTGTAGACTG | | |
| TMPRSS | AUCAUCUU | 980 | ACTCTTTGAAACTGTA | 1180 | human |
| 2_2641 | UGCCAAGU | | TCATCTTTGCCAAGTA | | |
| | AAGA | | AGAGTGGTGGCCT | | |
| TMPRSS | UCAGACAC | 981 | CTGTGTGATTGTGCCT | 1181 | mouse |
| 2_2641 | UAGACCUC | | CAGACACTAGACCTCT | | |
| | UUCC | | TCCAGATGGTTAG | | |
| TMPRSS | UGAAACUG | 982 | CTTCATTTAACTCTTT | 1182 | human |
| 2_2632 | UAUCAUCU | | GAAACTGTATCATCTT | | |
| | UUGC | | TGCCAAGTAAGAG | | |
| TMPRSS | UUUGAAAC | 983 | ACCTTCATTTAACTCT | 1183 | human |
| 2_2630 | UGUAUCAU | | TTGAAACTGTATCATC | | |
| | CUUU | | TTTGCCAAGTAAG | | |
| TMPRSS | AACUCUUU | 984 | AGTCCACCTTCATTTA | 1184 | human |
| 2_2625 | GAAACUGU | | ACTCTTTGAAACTGTA | | |
| | AUCA | | TCATCTTTGCCAA | | |
| TMPRSS | UUGGACUG | 985 | GGAGTTCACCTGCATT | 1185 | mouse |
| 2_2621 | UGUGAUUG | | TGGACTGTGTGATTGT | | |
| | UGCC | | GCCTCAGACACTA | | |
| TMPRSS | CCACCUUC | 986 | ATGTCTCCAAGTAGTC | 1186 | human |
| 2_2613 | AUUUAACU | | CACCTTCATTTAACTC | | |
| | CUUU | | TTTGAAACTGTAT | | |
| TMPRSS | UAGUCCAC | 987 | TTTGATGTCTCCAAGT | 1187 | human |
| 2_2609 | CUUCAUUU | | AGTCCACCTTCATTTA | | |
| | AACU | | ACTCTTTGAAACT | | |
| TMPRSS | UUUGAUGU | 988 | CCTGGAAACTTAGCTT | 1188 | human |
| 2_2594 | CUCCAAGU | | TTGATGTCTCCAAGTA | | |
| | AGUC | | GTCCACCTTCATT | | |
| TMPRSS | UUAGCUUU | 989 | AGTGCTCCTGGAAACT | 1189 | human |
| 2_2588 | UGAUGUCU | | TAGCTTTTGATGTCTC | | |
| | CCAA | | CAAGTAGTCCACC | | |
| TMPRSS | CCUGGAAA | 990 | TGCTACCTCAGTGCTC | 1190 | human |
| 2_2579 | CUUAGCUU | | CTGGAAACTTAGCTTT | | |
| | UUGA | | TGATGTCTCCAAG | | |
| TMPRSS | UACUACCU | 991 | TTGTGTTTCTTCTCTTA | 1191 | mouse |
| 2_2564 | CACUGCAC | | CTACCTCACTGCACCT | | |
| | CUGG | | GGACACTAGAGT | | |
| TMPRSS | UCUGGGUU | 992 | TTCAGTCACCTTGCTT | 1192 | mouse |
| 2_2543 | GUGUUUCU | | CTGGGTTGTGTTTCTT | | |
| | UCUC | | CTCTTACTACCTC | | |
| TMPRSS | UUUCCAUG | 993 | GTTTAAGGTACACTGT | 1193 | human |
| 2_2540 | UUAUGUUU | | TTCCATGTTATGTTTC | | |
| | CUAC | | TACACATTGCTAC | | |
| TMPRSS | UACACUGU | 994 | ATGCTCAGTTTAAGGT | 1194 | human |
| 2_2533 | UUCCAUGU | | ACACTGTTTCCATGTT | | |
| | UAUG | | ATGTTTCTACACA | | |
| TMPRSS | GUUUAAGG | 995 | AATCAAGGATGCTCA | 1195 | human |
| 2_2525 | UACACUGU | | GTTTAAGGTACACTGT | | |
| | UUCC | | TTCCATGTTATGTT | | |
| TMPRSS | AGUUUAAG | 996 | AAATCAAGGATGCTC | 1196 | human |
| 2_2524 | GUACACUG | | AGTTTAAGGTACACTG | | |
| | UUUC | | TTTCCATGTTATGT | | |
| TMPRSS | AUCAGAAU | 997 | TGGAGGCTCAGGTCC | 1197 | mouse |
| 2_2506 | CAGGGACU | | ATCAGAATCAGGGAC | | |
| | UGUG | | TTGTGATTTCAGTCA | | |
| TMPRSS | GGGGAAAU | 998 | AAATTGAGGTCCATG | 1198 | human |
| 2_2505 | CAAGGAUG | | GGGGAAATCAAGGAT | | |
| | CUCA | | GCTCAGTTTAAGGTA | | |
| TMPRSS | AGGUCCAU | 999 | ATCAAATGGAGGCTC | 1199 | mouse |
| 2_2500 | CAGAAUCA | | AGGTCCATCAGAATC | | |
| | GGGA | | AGGGACTTGTGATTT | | |
| TMPRSS | UCAGGUCC | 1000 | TAATCAAATGGAGGC | 1200 | mouse |
| 2_2498 | AUCAGAAU | | TCAGGTCCATCAGAAT | | |
| | CAGG | | CAGGGACTTGTGAT | | |
| TMPRSS | UGGAGCCU | 1001 | TGGCCTAGTACCTGAT | 1201 | mouse |
| 2_2463 | GUAUAGCU | | GGAGCCTGTATAGCTC | | |
| | CAGC | | AGCTAATCAAATG | | |
| TMPRSS | ACAGGCAU | 1002 | CTCTTAGCTTTGGCTA | 1202 | mouse |
| 2_2441 | GGCCUAGU | | CAGGCATGGCCTAGT | | |
| | ACCU | | ACCTGATGGAGCCT | | |
| TMPRSS | CCUGAAAC | 1003 | CTCAGCCTCTCAGAGC | 1203 | mouse |
| 2_2414 | UUACCUCU | | CTGAAACTTACCTCTT | | |
| | UAGC | | AGCTTTGGCTACA | | |
| TMPRSS | UCUCAGAG | 1004 | CTGTTTCTCTCAGCCT | 1204 | mouse |
| 2_2406 | CCUGAAAC | | CTCAGAGCCTGAAACT | | |
| | UUAC | | TACCTCTTAGCTT | | |
| TMPRSS | UGGGUGAG | 1005 | TGCCGGCATGTCCCTT | 1205 | mouse |
| 2_2341 | CUCUACAU | | GGGTGAGCTCTACATG | | |
| | GGUG | | GTGTTATTCAGTC | | |
| TMPRSS | AGAGCAAG | 1006 | CTAGGCAGATCTCTCA | 1206 | mouse |
| 2_2311 | AAGCUAAU | | GAGCAAGAAGCTAAT | | |
| | GCCG | | GCCGGCATGTCCCT | | |
| TMPRSS | UUCCCAAG | 1007 | AGGGTGATGGAGGCT | 1207 | mouse |
| 2_2280 | CUAAGGGC | | TTCCCAAGCTAAGGGC | | |
| | CUAG | | CTAGGCAGATCTCT | | |
| TMPRSS | AUAGACAG | 1008 | TTATGGGGTGAGAAT | 1208 | human |
| 2_2257 | UGCCCUUG | | ATAGACAGTGCCCTTG | | |
| | GUGC | | GTGCGAGGGAAGCA | | |
| TMPRSS | GGUGACGU | 1009 | CAGCCCTTCATGGGTG | 1209 | human |
| 2_2196 | GGUAGUCA | | GTGACGTGGTAGTCAC | | |
| | CUUG | | TTGTAAGGGGAAC | | |
| TMPRSS | UGGGCUGG | 1010 | ATCTGCTGTGCAGGTT | 1210 | mouse |
| 2_2165 | UCAUACUG | | GGGCTGGTCATACTGT | | |
| | UCAU | | CATGATTTCATTA | | |
| TMPRSS | GUUGGGCU | 1011 | AAATCTGCTGTGCAGG | 1211 | mouse |
| 2_2163 | GGUCAUAC | | TTGGGCTGGTCATACT | | |
| | UGUC | | GTCATGATTTCAT | | |
| TMPRSS | UGAGUAAC | 1012 | AAGGACTATGACCTCT | 1212 | mouse |
| 2_2047 | CUGAUGAC | | GAGTAACCTGATGAC | | |
| | CUGA | | CTGAGAAAGAGTAA | | |
| TMPRSS | GACCUCUG | 1013 | ATCACTAAGGACTATG | 1213 | mouse |
| 2_2041 | AGUAACCU | | ACCTCTGAGTAACCTG | | |
| | GAUG | | ATGACCTGAGAAA | | |
| TMPRSS | UGGAUCAC | 1014 | TGCTCAGGCCTTTTTT | 1214 | mouse |
| 2_2023 | UAAGGACU | | GGATCACTAAGGACT | | |
| | AUGA | | ATGACCTCTGAGTA | | |
| TMPRSS | UUGGAUCA | 1015 | ATGCTCAGGCCTTTTT | 1215 | mouse |
| 2_2022 | CUAAGGAC | | TGGATCACTAAGGACT | | |
| | UAUG | | ATGACCTCTGAGT | | |
| TMPRSS | ACUCUCAU | 1016 | GCAGAGGAGGGTGGC | 1216 | mouse |
| 2_1978 | GUUGGAAC | | ACTCTCATGTTGGAAC | | |
| | UUCU | | TTCTTTTGGGCTCA | | |
| TMPRSS | CUUUCCAG | 1017 | TCTTCCTGCTGAGTCC | 1217 | human |
| 2_1952 | GGGCCAAU | | TTTCCAGGGGCCAATT | | |
| | UUUG | | TTGGATGAGCATG | | |
| TMPRSS | ACCACCAG | 1018 | TTTGAACTCAGGGTCA | 1218 | human |
| 2_193 | CUAUUGGA | | CCACCAGCTATTGGAC | | |
| | CCUU | | CTTACTATGAAAA | | |
| TMPRSS | UGCCCCAU | 1019 | GAGAGGGGTGGAGGC | 1219 | human |
| 2_1924 | UGAGAUCU | | TGCCCCATTGAGATCT | | |
| | UCCU | | TCCTGCTGAGTCCT | | |
| TMPRSS | GAUGACCA | 1020 | GCTCCCACCAGAATG | 1220 | mouse |
| 2_1922 | GAUUCUGU | | GATGACCAGATTCTGT | | |
| | UGGG | | TGGGTTTGGGCACA | | |
| TMPRSS | CCCACCAG | 1021 | GCCACTTCTGCAGCTC | 1221 | mouse |
| 2_1910 | AAUGGAUG | | CCACCAGAATGGATG | | |
| | ACCA | | ACCAGATTCTGTTG | | |
| TMPRSS | UUCACUUU | 1022 | TGATTCGAAGGGCCTT | 1222 | mouse |
| 2_1821 | UAUUAAAC | | TCACTTTTATTAAACA | | |
| | AGUG | | GTGACTTGTTTGA | | |
| TMPRSS | UUUCACUU | 1023 | ATGATTCGAAGGGCCT | 1223 | mouse |
| 2_1820 | UUAUUAAA | | TTCACTTTTATTAAAC | | |
| | CAGU | | AGTGACTTGTTTG | | |
| TMPRSS | CGAAGGGC | 1024 | CCTTTTGAGATGATTC | 1224 | mouse |
| 2_1811 | CUUUCACU | | GAAGGGCCTTTCACTT | | |
| | UUUA | | TTATTAAACAGTG | | |
| TMPRSS | UUUGAGAU | 1025 | GTGCACAATGTACCTT | 1225 | mouse |
| 2_1799 | GAUUCGAA | | TTGAGATGATTCGAAG | | |
| | GGGC | | GGCCTTTCACTTT | | |
| TMPRSS | UGUACCUU | 1026 | ACTTCCTGTGCACAAT | 1226 | mouse |
| 2_1792 | UUGAGAUG | | GTACCTTTTGAGATGA | | |
| | AUUC | | TTCGAAGGGCCTT | | |
| TMPRSS | GUGCACAA | 1027 | GAATTTTAACTTCCTG | 1227 | mouse |
| 2_1784 | UGUACCUU | | TGCACAATGTACCTTT | | |
| | UUGA | | TGAGATGATTCGA | | |
| TMPRSS | CUGUGCAC | 1028 | CTGAATTTTAACTTCC | 1228 | mouse |
| 2_1782 | AAUGUACC | | TGTGCACAATGTACCT | | |
| | UUUU | | TTTGAGATGATTC | | |
| TMPRSS | CCUGAAUU | 1029 | CAAGAAAACCAAGGG | 1229 | mouse |
| 2_1766 | UUAACUUC | | CCTGAATTTTAACTTC | | |
| | CUGU | | CTGTGCACAATGTA | | |
| TMPRSS | UAAACAGU | 1030 | TCACTTCATTTTTATT | 1230 | human |
| 2_1764 | GAACUUGU | | AAACAGTGAACTTGTC | | |
| | CUGG | | TGGCTTTGGCACT | | |
| TMPRSS | UUAAACAG | 1031 | GTCACTTCATTTTTAT | 1231 | human |
| 2_1763 | UGAACUUG | | TAAACAGTGAACTTGT | | |
| | UCUG | | CTGGCTTTGGCAC | | |
| TMPRSS | AGGGCCUG | 1032 | TCTGCAAGAAAACCA | 1232 | mouse |
| 2_1762 | AAUUUUAA | | AGGGCCTGAATTTTAA | | |
| | CUUC | | CTTCCTGTGCACAA | | |
| TMPRSS | AAGGGCCU | 1033 | TTCTGCAAGAAAACC | 1233 | mouse |
| 2_1761 | GAAUUUUA | | AAGGGCCTGAATTTTA | | |
| | ACUU | | ACTTCCTGTGCACA | | |
| TMPRSS | CAAGAAAA | 1034 | TTCAACAACCTTCTGC | 1234 | mouse |
| 2_1751 | CCAAGGGC | | AAGAAAACCAAGGGC | | |
| | CUGA | | CTGAATTTTAACTT | | |
| TMPRSS | UGCAAGAA | 1035 | TCTTCAACAACCTTCT | 1235 | mouse |
| 2_1749 | AACCAAGG | | GCAAGAAAACCAAGG | | |
| | GCCU | | GCCTGAATTTTAAC | | |
| TMPRSS | CUGCAAGA | 1036 | GTCTTCAACAACCTTC | 1236 | mouse |
| 2_1748 | AAACCAAG | | TGCAAGAAAACCAAG | | |
| | GGCC | | GGCCTGAATTTTAA | | |
| TMPRSS | UUCUGCAA | 1037 | TTGTCTTCAACAACCT | 1237 | mouse |
| 2_1746 | GAAAACCA | | TCTGCAAGAAAACCA | | |
| | AGGG | | AGGGCCTGAATTTT | | |
| TMPRSS | AUUCAGAG | 1038 | TTACTCTTAGAGATGA | 1238 | human |
| 2_1740 | GUCACUUC | | TTCAGAGGTCACTTCA | | |
| | AUUU | | TTTTTATTAAACA | | |
| TMPRSS | UGAUUCAG | 1039 | ATTTACTCTTAGAGAT | 1239 | human |
| 2_1738 | AGGUCACU | | GATTCAGAGGTCACTT | | |
| | UCAU | | CATTTTTATTAAA | | |
| TMPRSS | UUAGAGAU | 1040 | GTGCATGATTTACTCT | 1240 | human |
| 2_1731 | GAUUCAGA | | TAGAGATGATTCAGA | | |
| | GGUC | | GGTCACTTCATTTT | | |
| TMPRSS | UUUACUCU | 1041 | CTTCCCCGTGCATGAT | 1241 | human |
| 2_1724 | UAGAGAUG | | TTACTCTTAGAGATGA | | |
| | AUUC | | TTCAGAGGTCACT | | |
| TMPRSS | UGUCCCAG | 1042 | TAATCCACGTGGCTTT | 1242 | mouse |
| 2_1716 | ACUUCCUU | | GTCCCAGACTTCCTTT | | |
| | UGUC | | GTCTTCAACAACC | | |
| TMPRSS | CCGUGCAU | 1043 | GCTGGTTTTGCTTCCC | 1243 | human |
| 2_1714 | GAUUUACU | | CGTGCATGATTTACTC | | |
| | CUUA | | TTAGAGATGATTC | | |
| TMPRSS | GGCUUUGU | 1044 | ACAGCTAATCCACGTG | 1244 | mouse |
| 2_1711 | CCCAGACU | | GCTTTGTCCCAGACTT | | |
| | UCCU | | CCTTTGTCTTCAA | | |
| TMPRSS | UUCCCCGU | 1045 | TGGGGCTGGTTTTGCT | 1245 | human |
| 2_1710 | GCAUGAUU | | TCCCCGTGCATGATTT | | |
| | UACU | | ACTCTTAGAGATG | | |
| TMPRSS | UUACAAGA | 1046 | GTCCTTGACGTCGTTT | 1246 | human |
| 2_1681 | AAACAAUG | | TACAAGAAAACAATG | | |
| | GGGC | | GGGCTGGTTTTGCT | | |
| TMPRSS | UUUACAAG | 1047 | CGTCCTTGACGTCGTT | 1247 | human |
| 2_1680 | AAAACAAU | | TTACAAGAAAACAAT | | |
| | GGGG | | GGGGCTGGTTTTGC | | |
| TMPRSS | UUUUACAA | 1048 | TCGTCCTTGACGTCGT | 1248 | human |
| 2_1679 | GAAAACAA | | TTTACAAGAAAACAA | | |
| | UGGG | | TGGGGCTGGTTTTG | | |
| TMPRSS | UUCGUCCU | 1049 | CTAATCCACATGGTCT | 1249 | human |
| 2_1663 | UGACGUCG | | TCGTCCTTGACGTCGT | | |
| | UUUU | | TTTACAAGAAAAC | | |
| TMPRSS | ACGGUAUU | 1050 | GTATACGGGAACGTG | 1250 | mouse |
| 2_1656 | UACAGAUU | | ACGGTATTTACAGATT | | |
| | GGAU | | GGATCTACCAGCAA | | |
| TMPRSS | GACGGUAU | 1051 | AGTATACGGGAACGT | 1251 | mouse |
| 2_1655 | UUACAGAU | | GACGGTATTTACAGAT | | |
| | UGGA | | TGGATCTACCAGCA | | |
| TMPRSS | ACGGGAAC | 1052 | TCAGACCTGGAGTATA | 1252 | mouse |
| 2_1645 | GUGACGGU | | CGGGAACGTGACGGT | | |
| | AUUU | | ATTTACAGATTGGA | | |
| TMPRSS | AUGGUAUU | 1053 | GTGTACGGGAATGTG | 1253 | human |
| 2_1604 | CACGGACU | | ATGGTATTCACGGACT | | |
| | GGAU | | GGATTTATCGACAA | | |
| TMPRSS | GAUGGUAU | 1054 | AGTGTACGGGAATGT | 1254 | human |
| 2_1603 | UCACGGAC | | GATGGTATTCACGGAC | | |
| | UGGA | | TGGATTTATCGACA | | |
| TMPRSS | GGGAAUGU | 1055 | AGACCAGGAGTGTAC | 1255 | human |
| 2_1595 | GAUGGUAU | | GGGAATGTGATGGTA | | |
| | UCAC | | TTCACGGACTGGATT | | |
| TMPRSS | ACGGGAAU | 1056 | ACAGACCAGGAGTGT | 1256 | human |
| 2_1593 | GUGAUGGU | | ACGGGAATGTGATGG | | |
| | AUUC | | TATTCACGGACTGGA | | |
| TMPRSS | GUUGAUAA | 1057 | TCATTACTCGATGCTG | 1257 | human |
| 2_158 | CAGCAAGA | | TTGATAACAGCAAGA | | |
| | UGGC | | TGGCTTTGAACTCA | | |
| TMPRSS | CUGUUGAU | 1058 | TATCATTACTCGATGC | 1258 | human |
| 2_156 | AACAGCAA | | TGTTGATAACAGCAA | | |
| | GAUG | | GATGGCTTTGAACT | | |
| TMPRSS | UCUGGCUG | 1059 | GATACAAGCTGGGGT | 1259 | human |
| 2_1559 | UGCCAAAG | | TCTGGCTGTGCCAAAG | | |
| | CUUA | | CTTACAGACCAGGA | | |
| TMPRSS | GGUUACUU | 1060 | CAGTGGAGGGCCGCT | 1260 | mouse |
| 2_1559 | UGAAGAAU | | GGTTACTTTGAAGAAT | | |
| | GGGA | | GGGATCTGGTGGCT | | |
| TMPRSS | UGUAAUAG | 1061 | ATCGAGCCCTCCAAAT | 1261 | mouse |
| 2_1452 | UAAAUACA | | GTAATAGTAAATACAT | | |
| | UAUA | | ATACAACAACCTA | | |
| TMPRSS | CCAAAUGU | 1062 | CCTTGATCGAGCCCTC | 1262 | mouse |
| 2_1447 | AAUAGUAA | | CAAATGTAATAGTAA | | |
| | AUAC | | ATACATATACAACA | | |
| TMPRSS | CUCCAAAU | 1063 | ACCCTTGATCGAGCCC | 1263 | mouse |
| 2_1445 | GUAAUAGU | | TCCAAATGTAATAGTA | | |
| | AAAU | | AATACATATACAA | | |
| TMPRSS | CGAGCCCU | 1064 | CATGGTACCCTTGATC | 1264 | mouse |
| 2_1439 | CCAAAUGU | | GAGCCCTCCAAATGTA | | |
| | AAUA | | ATAGTAAATACAT | | |
| TMPRSS | GAUCGAGC | 1065 | TGCCATGGTACCCTTG | 1265 | mouse |
| 2_1436 | CCUCCAAA | | ATCGAGCCCTCCAAAT | | |
| | UGUA | | GTAATAGTAAATA | | |
| TMPRSS | AUGUCUAU | 1066 | GATGCAACAGCAGAT | 1266 | human |
| 2_1413 | GACAACCU | | ATGTCTATGACAACCT | | |
| | GAUC | | GATCACACCAGCCA | | |
| TMPRSS | UCGGACGU | 1067 | GAGAAAGGGAAGACC | 1267 | mouse |
| 2_1404 | GUUGAAUG | | TCGGACGTGTTGAATG | | |
| | CUGC | | CTGCCATGGTACCC | | |
| TMPRSS | AACAGCAG | 1068 | GAGACACAGAGATGC | 1268 | human |
| 2_1403 | AUAUGUCU | | AACAGCAGATATGTCT | | |
| | AUGA | | ATGACAACCTGATC | | |
| TMPRSS | CUCGGACG | 1069 | TGAGAAAGGGAAGAC | 1269 | mouse |
| 2_1403 | UGUUGAAU | | CTCGGACGTGTTGAAT | | |
| | GCUG | | GCTGCCATGGTACC | | |
| TMPRSS | AGAUGCAA | 1070 | CTCATTGAGACACAG | 1270 | human |
| 2_1397 | CAGCAGAU | | AGATGCAACAGCAGA | | |
| | AUGU | | TATGTCTATGACAAC | | |
| TMPRSS | UGAGAAAG | 1071 | GTGGGGGGCCACCTA | 1271 | mouse |
| 2_1388 | GGAAGACC | | TGAGAAAGGGAAGAC | | |
| | UCGG | | CTCGGACGTGTTGAA | | |
| TMPRSS | AUACCUAU | 1072 | TATTGAACATTCCAGA | 1272 | human |
| 2_136 | CAUUACUC | | TACCTATCATTACTCG | | |
| | GAUG | | ATGCTGTTGATAA | | |
| TMPRSS | UCAGAAGU | 1073 | GAGAAAGGGAAGACC | 1273 | human |
| 2_1352 | GCUGAACG | | TCAGAAGTGCTGAAC | | |
| | CUGC | | GCTGCCAAGGTGCTT | | |
| TMPRSS | CUCAGAAG | 1074 | GGAGAAAGGGAAGAC | 1274 | human |
| 2_1351 | UGCUGAAC | | CTCAGAAGTGCTGAA | | |
| | GCUG | | CGCTGCCAAGGTGCT | | |
| TMPRSS | CCAGAUAC | 1075 | GTCATATTGAACATTC | 1275 | human |
| 2_132 | CUAUCAUU | | CAGATACCTATCATTA | | |
| | ACUC | | CTCGATGCTGTTG | | |
| TMPRSS | UCCAGAUA | 1076 | GGTCATATTGAACATT | 1276 | human |
| 2_131 | CCUAUCAU | | CCAGATACCTATCATT | | |
| | UACU | | ACTCGATGCTGTT | | |
| TMPRSS | UUGGCUUU | 1077 | AAGCTGCAGACACCTT | 1277 | mouse |
| 2_1287 | UAAUGAUC | | TGGCTTTTAATGATCT | | |
| | UAGU | | AGTGAAGCCAGTG | | |
| TMPRSS | UGAAGCUG | 1078 | ACGACATTGCTCTCAT | 1278 | mouse |
| 2_1270 | CAGACACC | | GAAGCTGCAGACACC | | |
| | UUUG | | TTTGGCTTTTAATG | | |
| TMPRSS | AACGACAU | 1079 | TCTAAGACCAAGAAT | 1279 | mouse |
| 2_1254 | UGCUCUCA | | AACGACATTGCTCTCA | | |
| | UGAA | | TGAAGCTGCAGACA | | |
| TMPRSS | GCAGAAGC | 1080 | TGCGCTGATGAAGCTG | 1280 | human |
| 2_1225 | CUCUGACU | | CAGAAGCCTCTGACTT | | |
| | UUCA | | TCAACGACCTAGT | | |
| TMPRSS | UUCCCAUC | 1081 | GGTAGAAAAAGTAAT | 1281 | mouse |
| 2_1220 | CAAAUUAC | | TTCCCATCCAAATTAC | | |
| | GACU | | GACTCTAAGACCAA | | |
| TMPRSS | GGAAGUAG | 1082 | TCTCTCATGTTCTATG | 1282 | mouse |
| 2_1191 | ACACCAGG | | GAAGTAGACACCAGG | | |
| | UAGA | | TAGAAAAAGTAATT | | |
| TMPRSS | UGGAAGUA | 1083 | GTCTCTCATGTTCTAT | 1283 | mouse |
| 2_1190 | GACACCAG | | GGAAGTAGACACCAG | | |
| | GUAG | | GTAGAAAAAGTAAT | | |
| TMPRSS | UGUUCUAU | 1084 | TGAGACAGTCTCTCAT | 1284 | mouse |
| 2_1183 | GGAAGUAG | | GTTCTATGGAAGTAGA | | |
| | ACAC | | CACCAGGTAGAAA | | |
| TMPRSS | UAUGACUC | 1085 | ATTTCTCATCCAAATT | 1285 | human |
| 2_1181 | CAAGACCA | | ATGACTCCAAGACCA | | |
| | AGAA | | AGAACAATGACATT | | |
| TMPRSS | UUCUCAUC | 1086 | AGTAGAAAAAGTGAT | 1286 | human |
| 2_1168 | CAAAUUAU | | TTCTCATCCAAATTAT | | |
| | GACU | | GACTCCAAGACCAA | | |
| TMPRSS | UGAGACAG | 1087 | CATTTGCGGGAATTCT | 1287 | mouse |
| 2_1168 | UCUCUCAU | | GAGACAGTCTCTCATG | | |
| | GUUC | | TTCTATGGAAGTA | | |
| TMPRSS | CUGAGACA | 1088 | GCATTTGCGGGAATTC | 1288 | mouse |
| 2_1167 | GUCUCUCA | | TGAGACAGTCTCTCAT | | |
| | UGUU | | GTTCTATGGAAGT | | |
| TMPRSS | UGCGGGAA | 1089 | GTACTGGACGGCATTT | 1289 | mouse |
| 2_1157 | UUCUGAGA | | GCGGGAATTCTGAGA | | |
| | CAGU | | CAGTCTCTCATGTT | | |
| TMPRSS | CAGGUCAU | 1090 | CCGCCTGGAGCGCGG | 1290 | human |
| 2_114 | AUUGAACA | | CAGGTCATATTGAACA | | |
| | UUCC | | TTCCAGATACCTAT | | |
| TMPRSS | GAGACAAU | 1091 | ATTTGCGGGGATTTTG | 1291 | human |
| 2_1117 | CUUUCAUG | | AGACAATCTTTCATGT | | |
| | UUCU | | TCTATGGAGCCGG | | |
| TMPRSS | CGCGGCAG | 1092 | GAGCGCCGCCTGGAG | 1292 | human |
| 2_109 | GUCAUAUU | | CGCGGCAGGTCATATT | | |
| | GAAC | | GAACATTCCAGATA | | |
| TMPRSS | AUGGCAUU | 1093 | ACCTCTTAACAATCCA | 1293 | human |
| 2_1087 | GGACGGCA | | TGGCATTGGACGGCAT | | |
| | UUUG | | TTGCGGGGATTTT | | |
| TMPRSS | CAUGGCAU | 1094 | AACCTCTTAACAATCC | 1294 | human |
| 2_1086 | UGGACGGC | | ATGGCATTGGACGGC | | |
| | AUUU | | ATTTGCGGGGATTT | | |

TABLE 11B
Host target IL-6 - 20 nucleotide targets and 45 nucleotide gene
target regions

| | | SEQ | | SEQ | |
|---|---|---|---|---|---|
| SEQUENCE | | ID | | ID | |
| ID | 20 nt Sequence | NO: | 45 nt Gene Region | NO: | Species |
| IL6_999 | UUGUAUUU | 1295 | AAAGAAATATTTATATTG | 1495 | human |
| | AUAUAAUG | | TATTTATATAATGTATAAA | | |
| | UAUA | | TGGTTTTT | | |
| IL6_998 | AUUGUAUU | 1296 | TAAAGAAATATTTATATT | 1496 | human |
| | UAUAUAAU | | GTATTTATATAATGTATAA | | |
| | GUAU | | ATGGTTTT | | |
| IL6_995 | UAUAUUGU | 1297 | TTTTAAAGAAATATTTATA | 1497 | human |
| | AUUUAUAU | | TTGTATTTATATAATGTAT | | |
| | AAUG | | AAATGGT | | |
| IL6_994 | UUAUAUUG | 1298 | TTTTTAAAGAAATATTTAT | 1498 | human |
| | UAUUUAUA | | ATTGTATTTATATAATGTA | | |
| | UAAU | | TAAATGG | | |
| IL6_989 | AAUAUUUA | 1299 | ACATATTTTTAAAGAAAT | 1499 | human |
| | UAUUGUAU | | ATTTATATTGTATTTATAT | | |
| | UUAU | | AATGTATA | | |
| IL6_984 | AAAGAAAU | 1300 | CTTATACATATTTTTAAAG | 1500 | human |
| | AUUUAUAU | | AAATATTTATATTGTATTT | | |
| | UGUA | | ATATAAT | | |
| IL6_982 | UUAAAGAA | 1301 | AACTTATACATATTTTTAA | 1501 | human |
| | AUAUUUAU | | AGAAATATTTATATTGTAT | | |
| | AUUG | | TTATATA | | |
| IL6_980 | UUUUAAAG | 1302 | CTAACTTATACATATTTTT | 1502 | human |
| | AAAUAUUU | | AAAGAAATATTTATATTG | | |
| | AUAU | | TATTTATA | | |
| IL6_963 | GGCUAACUU | 1303 | TTACCTCAAATAAATGGC | 1503 | human |
| | AUACAUAUU | | TAACTTATACATATTTTTA | | |
| | UU | | AAGAAATA | | |
| IL6_962 | UGGCUAACU | 1304 | CTTACCTCAAATAAATGG | 1504 | human |
| | UAUACAUAU | | CTAACTTATACATATTTTT | | |
| | UU | | AAAGAAAT | | |
| IL6_955 | AAAUAAAU | 1305 | GTGTAGGCTTACCTCAAA | 1505 | human |
| | GGCUAACUU | | TAAATGGCTAACTTATAC | | |
| | AUA | | ATATTTTTA | | |
| IL6_951 | CCUCAAAUA | 1306 | GAAAGTGTAGGCTTACCT | 1506 | human |
| | AAUGGCUAA | | CAAATAAATGGCTAACTT | | |
| | CU | | ATACATATT | | |
| IL6_950 | ACCUCAAAU | 1307 | GGAAAGTGTAGGCTTACC | 1507 | human |
| | AAAUGGCUA | | TCAAATAAATGGCTAACT | | |
| | AC | | TATACATAT | | |
| IL6_949 | UACCUCAAA | 1308 | TGGAAAGTGTAGGCTTAC | 1508 | human |
| | UAAAUGGCU | | CTCAAATAAATGGCTAAC | | |
| | AA | | TTATACATA | | |
| IL6_948 | UUACCUCAA | 1309 | TTGGAAAGTGTAGGCTTA | 1509 | human |
| | AUAAAUGGC | | CCTCAAATAAATGGCTAA | | |
| | UA | | CTTATACAT | | |
| IL6_947 | CUUACCUCA | 1310 | CTTGGAAAGTGTAGGCTT | 1510 | human |
| | AAUAAAUG | | ACCTCAAATAAATGGCTA | | |
| | GCU | | ACTTATACA | | |
| IL6_943 | UAGGCUUAC | 1311 | ATTTCTTGGAAAGTGTAG | 1511 | human |
| | CUCAAAUAA | | GCTTACCTCAAATAAATG | | |
| | AU | | GCTAACTTA | | |
| IL6_934 | UGGAAAGU | 1312 | AGCCAGATCATTTCTTGG | 1512 | human |
| | GUAGGCUUA | | AAAGTGTAGGCTTACCTC | | |
| | CCU | | AAATAAATG | | |
| IL6_932 | CUUGGAAAG | 1313 | AGAGCCAGATCATTTCTT | 1513 | human |
| | UGUAGGCUU | | GGAAAGTGTAGGCTTACC | | |
| | AC | | TCAAATAAA | | |
| IL6_917 | AGAGCCAGA | 1314 | AATATCCTTTGTTTCAGAG | 1514 | human |
| | UCAUUUCUU | | CCAGATCATTTCTTGGAA | | |
| | GG | | AGTGTAGG | | |
| IL6_912 | GUUUCAGAG | 1315 | GTTTGAATATCCTTTGTTT | 1515 | human |
| | CCAGAUCAU | | CAGAGCCAGATCATTTCTT | | |
| | UU | | GGAAAGT | | |
| IL6_894 | GCAGUUUGA | 1316 | ATGGAAAGTGGCTATGCA | 1516 | human |
| | AUAUCCUUU | | GTTTGAATATCCTTTGTTT | | |
| | GU | | CAGAGCCA | | |
| IL6_878 | AAUGGAAA | 1317 | TAGTTTTGAAATAATAAT | 1517 | human |
| | GUGGCUAUG | | GGAAAGTGGCTATGCAGT | | |
| | CAG | | TTGAATATC | | |
| IL6_870 | GAAAUAAU | 1318 | TTATGTATTAGTTTTGAAA | 1518 | human |
| | AAUGGAAA | | TAATAATGGAAAGTGGCT | | |
| | GUGG | | ATGCAGTT | | |
| IL6_867 | UUUGAAAU | 1319 | ATTTTATGTATTAGTTTTG | 1519 | human |
| | AAUAAUGG | | AAATAATAATGGAAAGTG | | |
| | AAAG | | GCTATGCA | | |
| IL6_858 | UGUAUUAG | 1320 | ACTTGAAACATTTTATGTA | 1520 | human |
| | UUUUGAAA | | TTAGTTTTGAAATAATAAT | | |
| | UAAU | | GGAAAGT | | |
| IL6_856 | UAUGUAUU | 1321 | CCACTTGAAACATTTTATG | 1521 | human |
| | AGUUUUGA | | TATTAGTTTTGAAATAATA | | |
| | AAUA | | ATGGAAA | | |
| IL6_851 | CAUUUUAUG | 1322 | AAGTACCACTTGAAACAT | 1522 | human |
| | UAUUAGUU | | TTTATGTATTAGTTTTGAA | | |
| | UUG | | ATAATAAT | | |
| IL6_843 | ACUUGAAAC | 1323 | TTTTTAAGAAGTACCACTT | 1523 | human |
| | AUUUUAUG | | GAAACATTTTATGTATTAG | | |
| | UAU | | TTTTGAA | | |
| IL6_840 | ACCACUUGA | 1324 | ATATTTTTAAGAAGTACC | 1524 | human |
| | AACAUUUUA | | ACTTGAAACATTTTATGTA | | |
| | UG | | TTAGTTTT | | |
| IL6_839 | UACCACUUG | 1325 | TATATTTTTAAGAAGTACC | 1525 | human |
| | AAACAUUUU | | ACTTGAAACATTTTATGTA | | |
| | AU | | TTAGTTT | | |
| IL6_838 | GUACCACUU | 1326 | TTATATTTTTAAGAAGTAC | 1526 | human |
| | GAAACAUUU | | CACTTGAAACATTTTATGT | | |
| | UA | | ATTAGTT | | |
| IL6_831 | UUAAGAAG | 1327 | GTCATATTTATATTTTTAA | 1527 | human |
| | UACCACUUG | | GAAGTACCACTTGAAACA | | |
| | AAA | | TTTTATGT | | |
| IL6_830 | UUUAAGAA | 1328 | AGTCATATTTATATTTTTA | 1528 | human |
| | GUACCACUU | | AGAAGTACCACTTGAAAC | | |
| | GAA | | ATTTTATG | | |
| IL6_829 | UUUUAAGA | 1329 | AAGTCATATTTATATTTTT | 1529 | human |
| | AGUACCACU | | AAGAAGTACCACTTGAAA | | |
| | UGA | | CATTTTAT | | |
| IL6_809 | UAUGUAAG | 1330 | GAAGCTGAGTTAATTTAT | 1530 | human |
| | UCAUAUUUA | | GTAAGTCATATTTATATTT | | |
| | UAU | | TTAAGAAG | | |
| IL6_808 | UUAUGUAA | 1331 | TGAAGCTGAGTTAATTTAT | 1531 | human |
| | GUCAUAUUU | | GTAAGTCATATTTATATTT | | |
| | AUA | | TTAAGAA | | |
| IL6_805 | AAUUUAUG | 1332 | ATGTGAAGCTGAGTTAAT | 1532 | human |
| | UAAGUCAUA | | TTATGTAAGTCATATTTAT | | |
| | UUU | | ATTTTTAA | | |
| IL6_804 | UAAUUUAU | 1333 | TATGTGAAGCTGAGTTAA | 1533 | human |
| | GUAAGUCAU | | TTTATGTAAGTCATATTTA | | |
| | AUU | | TATTTTTA | | |
| IL6_803 | UUAAUUUA | 1334 | ATATGTGAAGCTGAGTTA | 1534 | human |
| | UGUAAGUCA | | ATTTATGTAAGTCATATTT | | |
| | UAU | | ATATTTTT | | |
| IL6_801 | AGUUAAUU | 1335 | AAATATGTGAAGCTGAGT | 1535 | human |
| | UAUGUAAG | | TAATTTATGTAAGTCATAT | | |
| | UCAU | | TTATATTT | | |
| IL6_800 | GAGUUAAU | 1336 | TAAATATGTGAAGCTGAG | 1536 | human |
| | UUAUGUAA | | TTAATTTATGTAAGTCATA | | |
| | GUCA | | TTTATATT | | |
| IL6_799 | UGAGUUAA | 1337 | TTAAATATGTGAAGCTGA | 1537 | human |
| | UUUAUGUA | | GTTAATTTATGTAAGTCAT | | |
| | AGUC | | ATTTATAT | | |
| IL6_798 | CUGAGUUAA | 1338 | TTTAAATATGTGAAGCTG | 1538 | human |
| | UUUAUGUA | | AGTTAATTTATGTAAGTCA | | |
| | AGU | | TATTTATA | | |
| IL6_794 | GAAGCUGAG | 1339 | AATATTTAAATATGTGAA | 1539 | human |
| | UUAAUUUA | | GCTGAGTTAATTTATGTAA | | |
| | UGU | | GTCATATT | | |
| IL6_793 | UGAAGCUGA | 1340 | TAATATTTAAATATGTGA | 1540 | human |
| | GUUAAUUU | | AGCTGAGTTAATTTATGTA | | |
| | AUG | | AGTCATAT | | |
| IL6_792 | GUGAAGCUG | 1341 | TTAATATTTAAATATGTGA | 1541 | human |
| | AGUUAAUU | | AGCTGAGTTAATTTATGTA | | |
| | UAU | | AGTCATA | | |
| IL6_790 | AUGUGAAGC | 1342 | TATTAATATTTAAATATGT | 1542 | human |
| | UGAGUUAA | | GAAGCTGAGTTAATTTAT | | |
| | UUU | | GTAAGTCA | | |
| IL6_788 | AUAUGUGA | 1343 | TTTATTAATATTTAAATAT | 1543 | human |
| | AGCUGAGUU | | GTGAAGCTGAGTTAATTT | | |
| | AAU | | ATGTAAGT | | |
| IL6_784 | UUAAAUAU | 1344 | TTAATTTATTAATATTTAA | 1544 | human |
| | GUGAAGCUG | | ATATGTGAAGCTGAGTTA | | |
| | AGU | | ATTTATGT | | |
| IL6_783 | UUUAAAUA | 1345 | TTTAATTTATTAATATTTA | 1545 | human |
| | UGUGAAGCU | | AATATGTGAAGCTGAGTT | | |
| | GAG | | AATTTATG | | |
| IL6_782 | AUUUAAAU | 1346 | TTTTAATTTATTAATATTT | 1546 | human |
| | AUGUGAAGC | | AAATATGTGAAGCTGAGT | | |
| | UGA | | TAATTTAT | | |
| IL6_778 | UAAUAUUU | 1347 | TTATTTTTAATTTATTAAT | 1547 | human |
| | AAAUAUGU | | ATTTAAATATGTGAAGCT | | |
| | GAAG | | GAGTTAAT | | |
| IL6_776 | AUUAAUAU | 1348 | AATTATTTTTAATTTATTA | 1548 | human |
| | UUAAAUAU | | ATATTTAAATATGTGAAG | | |
| | GUGA | | CTGAGTTA | | |
| IL6_774 | UUAUUAAU | 1349 | TTAATTATTTTTAATTTAT | 1549 | human |
| | AUUUAAAU | | TAATATTTAAATATGTGA | | |
| | AUGU | | AGCTGAGT | | |
| IL6_770 | UAAUUUAU | 1350 | TATTTTAATTATTTTTAAT | 1550 | human |
| | UAAUAUUU | | TTATTAATATTTAAATATG | | |
| | AAAU | | TGAAGCT | | |
| IL6_768 | UUUAAUUU | 1351 | ACTATTTTAATTATTTTTA | 1551 | human |
| | AUUAAUAU | | ATTTATTAATATTTAAATA | | |
| | UUAA | | TGTGAAG | | |
| IL6_747 | UAGGACACU | 1352 | TAAAAGTATGAGCGTTAG | 1552 | human |
| | AUUUUAAU | | GACACTATTTTAATTATTT | | |
| | UAU | | TTAATTTA | | |
| IL6_746 | UUAGGACAC | 1353 | CTAAAAGTATGAGCGTTA | 1553 | human |
| | UAUUUUAA | | GGACACTATTTTAATTATT | | |
| | UUA | | TTTAATTT | | |
| IL6_745 | GUUAGGACA | 1354 | ACTAAAAGTATGAGCGTT | 1554 | human |
| | CUAUUUUAA | | AGGACACTATTTTAATTAT | | |
| | UU | | TTTTAATT | | |
| IL6_743 | GCGUUAGGA | 1355 | GAACTAAAAGTATGAGCG | 1555 | human |
| | CACUAUUUU | | TTAGGACACTATTTTAATT | | |
| | AA | | ATTTTTAA | | |
| IL6_742 | AGCGUUAGG | 1356 | AGAACTAAAAGTATGAGC | 1556 | human |
| | ACACUAUUU | | GTTAGGACACTATTTTAAT | | |
| | UA | | TATTTTTA | | |
| IL6_741 | GAGCGUUAG | 1357 | GAGAACTAAAAGTATGAG | 1557 | human |
| | GACACUAUU | | CGTTAGGACACTATTTTAA | | |
| | UU | | TTATTTTT | | |
| IL6_740 | UGAGCGUUA | 1358 | GGAGAACTAAAAGTATGA | 1558 | human |
| | GGACACUAU | | GCGTTAGGACACTATTTTA | | |
| | UU | | ATTATTTT | | |
| IL6_739 | AUGAGCGUU | 1359 | TGGAGAACTAAAAGTATG | 1559 | human |
| | AGGACACUA | | AGCGTTAGGACACTATTTT | | |
| | UU | | AATTATTT | | |
| IL6_731 | CUAAAAGUA | 1360 | GTTCTCTATGGAGAACTA | 1560 | human |
| | UGAGCGUUA | | AAAGTATGAGCGTTAGGA | | |
| | GG | | CACTATTTT | | |
| IL6_723 | AUGGAGAAC | 1361 | CTTATGTTGTTCTCTATGG | 1561 | human |
| | UAAAAGUA | | AGAACTAAAAGTATGAGC | | |
| | UGA | | GTTAGGAC | | |
| IL6_717 | UUCUCUAUG | 1362 | ACAGAACTTATGTTGTTCT | 1562 | human |
| | GAGAACUAA | | CTATGGAGAACTAAAAGT | | |
| | AA | | ATGAGCGT | | |
| IL6_697 | UGGGCACAG | 1363 | CAGAAACCTGTCCACTGG | 1563 | human |
| | AACUUAUGU | | GCACAGAACTTATGTTGTT | | |
| | UG | | CTCTATGG | | |
| IL6_691 | GUCCACUGG | 1364 | TCTGGTCAGAAACCTGTC | 1564 | human |
| | GCACAGAAC | | CACTGGGCACAGAACTTA | | |
| | UU | | TGTTGTTCT | | |
| IL6_654 | GUUGUUAA | 1365 | GCACCTCAGATTGTTGTTG | 1565 | human |
| | UGGGCAUUC | | TTAATGGGCATTCCTTCTT | | |
| | CUU | | CTGGTCA | | |
| IL6_629 | UGUAGCAUG | 1366 | GGGCTCTTCGGCAAATGT | 1566 | human |
| | GGCACCUCA | | AGCATGGGCACCTCAGAT | | |
| | GA | | TGTTGTTGT | | |
| IL6_580 | CGCAGCUUU | 1367 | ACTCATCTCATTCTGCGCA | 1567 | human |
| | AAGGAGUUC | | GCTTTAAGGAGTTCCTGC | | |
| | CU | | AGTCCAGC | | |
| IL6_58 | CCAGCUAUG | 1368 | CTCCCCTCCAGGAGCCCA | 1568 | human |
| | AACUCCUUC | | GCTATGAACTCCTTCTCCA | | |
| | UC | | CAATACCC | | |
| IL6_561 | GACAACUCA | 1369 | GTGGCTGCAGGACATGAC | 1569 | human |
| | UCUCAUUCU | | AACTCATCTCATTCTGCGC | | |
| | GC | | AGCTTTAA | | |
| IL6_54 | GAGCCCAGC | 1370 | CTATCTCCCCTCCAGGAGC | 1570 | human |
| | UAUGAACUC | | CCAGCTATGAACTCCTTCT | | |
| | CU | | CCACAAT | | |
| IL6_427 | AUGAGUACA | 1371 | GCCAGAGCTGTGCAGATG | 1571 | human |
| | AAAGUCCUG | | AGTACAAAAGTCCTGATC | | |
| | AU | | CAGTTCCTG | | |
| IL6_423 | GCAGAUGAG | 1372 | ACAAGCCAGAGCTGTGCA | 1572 | human |
| | UACAAAAGU | | GATGAGTACAAAAGTCCT | | |
| | CC | | GATCCAGTT | | |
| IL6_404 | AGGAACAAG | 1373 | GATTTGAGAGTAGTGAGG | 1573 | human |
| | CCAGAGCUG | | AACAAGCCAGAGCTGTGC | | |
| | UG | | AGATGAGTA | | |
| IL6_382 | CAGAACAGA | 1374 | TACCTAGAGTACCTCCAG | 1574 | human |
| | UUUGAGAG | | AACAGATTTGAGAGTAGT | | |
| | UAG | | GAGGAACAA | | |
| IL6_381 | CCAGAACAG | 1375 | ATACCTAGAGTACCTCCA | 1575 | human |
| | AUUUGAGA | | GAACAGATTTGAGAGTAG | | |
| | GUA | | TGAGGAACA | | |
| IL6_352 | UUGGAGUU | 1376 | ATCATCACTGGTCTTTTGG | 1576 | human |
| | UGAGGUAU | | AGTTTGAGGTATACCTAG | | |
| | ACCU | | AGTACCTC | | |
| IL6_343 | ACUGGUCUU | 1377 | CTGGTGAAAATCATCACT | 1577 | human |
| | UUGGAGUU | | GGTCTTTTGGAGTTTGAGG | | |
| | UGA | | TATACCTA | | |
| IL6_339 | CAUCACUGG | 1378 | TTGCCTGGTGAAAATCAT | 1578 | human |
| | UCUUUUGGA | | CACTGGTCTTTTGGAGTTT | | |
| | GU | | GAGGTATA | | |
| IL6_331 | GUGAAAAUC | 1379 | GAGGAGACTTGCCTGGTG | 1579 | human |
| | AUCACUGGU | | AAAATCATCACTGGTCTTT | | |
| | CU | | TGGAGTTT | | |
| IL6_325 | UGCCUGGUG | 1380 | TTCAATGAGGAGACTTGC | 1580 | human |
| | AAAAUCAUC | | CTGGTGAAAATCATCACT | | |
| | AC | | GGTCTTTTG | | |
| IL6_324 | UUGCCUGGU | 1381 | ATTCAATGAGGAGACTTG | 1581 | human |
| | GAAAAUCAU | | CCTGGTGAAAATCATCAC | | |
| | CA | | TGGTCTTTT | | |
| IL6_311 | UCAAUGAGG | 1382 | GCTTCCAATCTGGATTCAA | 1582 | human |
| | AGACUUGCC | | TGAGGAGACTTGCCTGGT | | |
| | UG | | GAAAATCA | | |
| IL6_309 | AUUCAAUGA | 1383 | ATGCTTCCAATCTGGATTC | 1583 | human |
| | GGAGACUUG | | AATGAGGAGACTTGCCTG | | |
| | CC | | GTGAAAAT | | |
| IL6_296 | GCUUCCAAU | 1384 | CTGAAAAAGATGGATGCT | 1584 | human |
| | CUGGAUUCA | | TCCAATCTGGATTCAATG | | |
| | AU | | AGGAGACTT | | |
| IL6_248 | UGGCAGAAA | 1385 | GCAGCAAAGAGGCACTGG | 1585 | human |
| | ACAACCUGA | | CAGAAAACAACCTGAACC | | |
| | AC | | TTCCAAAGA | | |
| IL6_217 | AGUAACAUG | 1386 | GAGACATGTAACAAGAGT | 1586 | human |
| | UGUGAAAGC | | AACATGTGTGAAAGCAGC | | |
| | AG | | AAAGAGGCA | | |
| IL6_210 | UAACAAGAG | 1387 | GAGAAAGGAGACATGTAA | 1587 | human |
| | UAACAUGUG | | CAAGAGTAACATGTGTGA | | |
| | UG | | AAGCAGCAA | | |
| IL6_209 | GUAACAAGA | 1388 | TGAGAAAGGAGACATGTA | 1588 | human |
| | GUAACAUGU | | ACAAGAGTAACATGTGTG | | |
| | GU | | AAAGCAGCA | | |
| IL6_203 | AGACAUGUA | 1389 | CAGCCCTGAGAAAGGAGA | 1589 | human |
| | ACAAGAGUA | | CATGTAACAAGAGTAACA | | |
| | AC | | TGTGTGAAA | | |
| IL6_201 | GGAGACAUG | 1390 | CTCAGCCCTGAGAAAGGA | 1590 | human |
| | UAACAAGAG | | GACATGTAACAAGAGTAA | | |
| | UA | | CATGTGTGA | | |
| IL6_1008 | UAUAAUGU | 1391 | TTTATATTGTATTTATATA | 1591 | human |
| | AUAAAUGG | | ATGTATAAATGGTTTTTAT | | |
| | UUUU | | ACCAATA | | |
| IL6_1007 | AUAUAAUG | 1392 | ATTTATATTGTATTTATAT | 1592 | human |
| | UAUAAAUG | | AATGTATAAATGGTTTTTA | | |
| | GUUU | | TACCAAT | | |
| IL6_1006 | UAUAUAAU | 1393 | TATTTATATTGTATTTATA | 1593 | human |
| | GUAUAAAU | | TAATGTATAAATGGTTTTT | | |
| | GGUU | | ATACCAA | | |
| IL6_1000 | UGUAUUUA | 1394 | AAGAAATATTTATATTGT | 1594 | human |
| | UAUAAUGU | | ATTTATATAATGTATAAAT | | |
| | AUAA | | GGTTTTTA | | |
| IL6_937 | UGUUAUAU | 1395 | AAGTGTCACTTGAAATGT | 1595 | mouse |
| | GUUAUAGU | | TATATGTTATAGTTTTGAA | | |
| | UUUG | | ATGATAAC | | |
| IL6_402 | UUGCCUAUU | 1396 | ATATAATCAGGAAATTTG | 1596 | mouse |
| | GAAAAUUUC | | CCTATTGAAAATTTCCTCT | | |
| | CU | | GGTCTTCT | | |
| IL6_1044 | UUGCUAAUU | 1397 | GTTTACCTCAATGAATTGC | 1597 | mouse |
| | UAAAUAUG | | TAATTTAAATATGTTTTTA | | |
| | UUU | | AAGAAAT | | |
| IL6_891 | UAAUUUAU | 1398 | AAGTAAACTTTAAGTTAA | 1598 | mouse |
| | GAUUGAUA | | TTTATGATTGATATTTATT | | |
| | UUUA | | ATTTTTAT | | |
| IL6_855 | UAAUUUAU | 1399 | TATTTTAATTATTTTTAAT | 1599 | mouse |
| | UGAUAAUU | | TTATTGATAATTTAAATAA | | |
| | UAAA | | GTAAACT | | |
| IL6_1036 | UCAAUGAAU | 1400 | ATGTATAAGTTTACCTCAA | 1600 | mouse |
| | UGCUAAUUU | | TGAATTGCTAATTTAAATA | | |
| | AA | | TGTTTTT | | |
| IL6_886 | UAAGUUAA | 1401 | TAAATAAGTAAACTTTAA | 1601 | mouse |
| | UUUAUGAU | | GTTAATTTATGATTGATAT | | |
| | UGAU | | TTATTATT | | |
| IL6_884 | UUUAAGUU | 1402 | TTTAAATAAGTAAACTTTA | 1602 | mouse |
| | AAUUUAUG | | AGTTAATTTATGATTGATA | | |
| | AUUG | | TTTATTA | | |
| IL6_827 | AAUGUUGG | 1403 | AGAACTGACAATATGAAT | 1603 | mouse |
| | GACACUAUU | | GTTGGGACACTATTTTAAT | | |
| | UUA | | TATTTTTA | | |
| IL6_1029 | GUUUACCUC | 1404 | TCTTGGAATGTATAAGTTT | 1604 | mouse |
| | AAUGAAUU | | ACCTCAATGAATTGCTAA | | |
| | GCU | | TTTAAATA | | |
| IL6_1016 | UUGGAAUG | 1405 | TAGCCAGATGGTTTCTTGG | 1605 | mouse |
| | UAUAAGUU | | AATGTATAAGTTTACCTCA | | |
| | UACC | | ATGAATT | | |
| IL6_779 | UGUCAGGUA | 1406 | GAAAATATATCCTGTTGTC | 1606 | mouse |
| | UCUGACUUA | | AGGTATCTGACTTATGTTG | | |
| | UG | | TTCTCTA | | |
| IL6_1066 | AAAGAAAUC | 1407 | TTTAAATATGTTTTTAAAG | 1607 | mouse |
| | UUUGUGAU | | AAATCTTTGTGATGTATTT | | |
| | GUA | | TTATAAT | | |
| IL6_854 | UUAAUUUA | 1408 | CTATTTTAATTATTTTTAA | 1608 | mouse |
| | UUGAUAAU | | TTTATTGATAATTTAAATA | | |
| | UUAA | | AGTAAAC | | |
| IL6_830 | GUUGGGACA | 1409 | ACTGACAATATGAATGTT | 1609 | mouse |
| | CUAUUUUAA | | GGGACACTATTTTAATTAT | | |
| | UU | | TTTTAATT | | |
| IL6_1089 | UUAUAAUG | 1410 | CTTTGTGATGTATTTTTAT | 1610 | mouse |
| | UUUAGACUG | | AATGTTTAGACTGTCTTCA | | |
| | UCU | | AACAAAT | | |
| IL6_931 | UUGAAAUG | 1411 | TTTATGAAGTGTCACTTGA | 1611 | mouse |
| | UUAUAUGU | | AATGTTATATGTTATAGTT | | |
| | UAUA | | TTGAAAT | | |
| IL6_538 | AACCAAGAG | 1412 | CTAATTCATATCTTCAACC | 1612 | mouse |
| | GUAAAAGA | | AAGAGGTAAAAGATTTAC | | |
| | UUU | | ATAAAATA | | |
| IL6_887 | AAGUUAAU | 1413 | AAATAAGTAAACTTTAAG | 1613 | mouse |
| | UUAUGAUU | | TTAATTTATGATTGATATT | | |
| | GAUA | | TATTATTT | | |
| IL6_408 | AUUGAAAA | 1414 | TCAGGAAATTTGCCTATTG | 1614 | mouse |
| | UUUCCUCUG | | AAAATTTCCTCTGGTCTTC | | |
| | GUC | | TGGAGTA | | |
| IL6_235 | GGCUUAAUU | 1415 | ACTTCACAAGTCGGAGGC | 1615 | mouse |
| | ACACAUGUU | | TTAATTACACATGTTCTCT | | |
| | CU | | GGGAAATC | | |
| IL6_725 | CUAAGCAUA | 1416 | CCTAGTGCGTTATGCCTAA | 1616 | mouse |
| | UCAGUUUGU | | GCATATCAGTTTGTGGAC | | |
| | GG | | ATTCCTCA | | |
| IL6_401 | UUUGCCUAU | 1417 | GATATAATCAGGAAATTT | 1617 | mouse |
| | UGAAAAUU | | GCCTATTGAAAATTTCCTC | | |
| | UCC | | TGGTCTTC | | |
| IL6_973 | AUCUAUUUG | 1418 | AATGATAACCTAAAAATC | 1618 | mouse |
| | AUAUAAAU | | TATTTGATATAAATATTCT | | |
| | AUU | | GTTACCTA | | |
| IL6_926 | GUCACUUGA | 1419 | TTATTTTTATGAAGTGTCA | 1619 | mouse |
| | AAUGUUAU | | CTTGAAATGTTATATGTTA | | |
| | AUG | | TAGTTTT | | |
| IL6_862 | UUGAUAAU | 1420 | ATTATTTTTAATTTATTGA | 1620 | mouse |
| | UUAAAUAA | | TAATTTAAATAAGTAAAC | | |
| | GUAA | | TTTAAGTT | | |
| IL6_868 | AUUUAAAU | 1421 | TTTAATTTATTGATAATTT | 1621 | mouse |
| | AAGUAAACU | | AAATAAGTAAACTTTAAG | | |
| | UUA | | TTAATTTA | | |
| IL6_869 | UUUAAAUA | 1422 | TTAATTTATTGATAATTTA | 1622 | mouse |
| | AGUAAACUU | | AATAAGTAAACTTTAAGT | | |
| | UAA | | TAATTTAT | | |
| IL6_885 | UUAAGUUA | 1423 | TTAAATAAGTAAACTTTA | 1623 | mouse |
| | AUUUAUGA | | AGTTAATTTATGATTGATA | | |
| | UUGA | | TTTATTAT | | |
| IL6_871 | UAAAUAAG | 1424 | AATTTATTGATAATTTAAA | 1624 | mouse |
| | UAAACUUUA | | TAAGTAAACTTTAAGTTA | | |
| | AGU | | ATTTATGA | | |
| IL6_652 | UUCAUCUUG | 1425 | ACCAAGACCATCCAATTC | 1625 | mouse |
| | AAAUCACUU | | ATCTTGAAATCACTTGAA | | |
| | GA | | GAATTTCTA | | |
| IL6_816 | CUGACAAUA | 1426 | GTTCTCTACGAAGAACTG | 1626 | mouse |
| | UGAAUGUU | | ACAATATGAATGTTGGGA | | |
| | GGG | | CACTATTTT | | |
| IL6_802 | UUCUCUACG | 1427 | ATCTGACTTATGTTGTTCT | 1627 | mouse |
| | AAGAACUGA | | CTACGAAGAACTGACAAT | | |
| | CA | | ATGAATGT | | |
| IL6_761 | UCAGAAAAU | 1428 | CATTCCTCACTGTGGTCAG | 1628 | mouse |
| | AUAUCCUGU | | AAAATATATCCTGTTGTCA | | |
| | UG | | GGTATCT | | |
| IL6_1007 | GAUGGUUUC | 1429 | TCTGTTACCTAGCCAGATG | 1629 | mouse |
| | UUGGAAUG | | GTTTCTTGGAATGTATAAG | | |
| | UAU | | TTTACCT | | |
| IL6_923 | AGUGUCACU | 1430 | TTATTATTTTTATGAAGTG | 1630 | mouse |
| | UGAAAUGU | | TCACTTGAAATGTTATATG | | |
| | UAU | | TTATAGT | | |
| IL6_978 | UUUGAUAU | 1431 | TAACCTAAAAATCTATTTG | 1631 | mouse |
| | AAAUAUUCU | | ATATAAATATTCTGTTACC | | |
| | GUU | | TAGCCAG | | |
| IL6_1063 | UUUAAAGA | 1432 | TAATTTAAATATGTTTTTA | 1632 | mouse |
| | AAUCUUUGU | | AAGAAATCTTTGTGATGT | | |
| | GAU | | ATTTTTAT | | |
| IL6_953 | UUUGAAAU | 1433 | GTTATATGTTATAGTTTTG | 1633 | mouse |
| | GAUAACCUA | | AAATGATAACCTAAAAAT | | |
| | AAA | | CTATTTGA | | |
| IL6_389 | AUAAUCAGG | 1434 | GCTACCAAACTGGATATA | 1634 | mouse |
| | AAAUUUGCC | | ATCAGGAAATTTGCCTATT | | |
| | UA | | GAAAATTT | | |
| IL6_541 | CAAGAGGUA | 1435 | ATTCATATCTTCAACCAAG | 1635 | mouse |
| | AAAGAUUU | | AGGTAAAAGATTTACATA | | |
| | ACA | | AAATAGTC | | |
| IL6_268 | GAAAUGAG | 1436 | CTCTGGGAAATCGTGGAA | 1636 | mouse |
| | AAAAGAGU | | ATGAGAAAAGAGTTGTGC | | |
| | UGUG | | AATGGCAAT | | |
| IL6_880 | AAACUUUAA | 1437 | ATAATTTAAATAAGTAAA | 1637 | mouse |
| | GUUAAUUU | | CTTTAAGTTAATTTATGAT | | |
| | AUG | | TGATATTT | | |
| IL6_861 | AUUGAUAA | 1438 | AATTATTTTTAATTTATTG | 1638 | mouse |
| | UUUAAAUA | | ATAATTTAAATAAGTAAA | | |
| | AGUA | | CTTTAAGT | | |
| IL6_875 | UAAGUAAAC | 1439 | TATTGATAATTTAAATAA | 1639 | mouse |
| | UUUAAGUU | | GTAAACTTTAAGTTAATTT | | |
| | AAU | | ATGATTGA | | |
| IL6_256 | UGGGAAAUC | 1440 | ATTACACATGTTCTCTGGG | 1640 | mouse |
| | GUGGAAAU | | AAATCGTGGAAATGAGAA | | |
| | GAG | | AAGAGTTG | | |
| IL6_351 | AGAGAUACA | 1441 | CAATCTGAAACTTCCAGA | 1641 | mouse |
| | AAGAAAUG | | GATACAAAGAAATGATGG | | |
| | AUG | | ATGCTACCA | | |
| IL6_824 | AUGAAUGU | 1442 | CGAAGAACTGACAATATG | 1642 | mouse |
| | UGGGACACU | | AATGTTGGGACACTATTTT | | |
| | AUU | | AATTATTT | | |
| IL6_332 | AAAACAAUC | 1443 | ATGATGCACTTGCAGAAA | 1643 | mouse |
| | UGAAACUUC | | ACAATCTGAAACTTCCAG | | |
| | CA | | AGATACAAA | | |
| IL6_403 | UGCCUAUUG | 1444 | TATAATCAGGAAATTTGC | 1644 | mouse |
| | AAAAUUUCC | | CTATTGAAAATTTCCTCTG | | |
| | UC | | GTCTTCTG | | |
| IL6_762 | CAGAAAAUA | 1445 | ATTCCTCACTGTGGTCAGA | 1645 | mouse |
| | UAUCCUGUU | | AAATATATCCTGTTGTCAG | | |
| | GU | | GTATCTG | | |
| IL6_879 | UAAACUUUA | 1446 | GATAATTTAAATAAGTAA | 1646 | mouse |
| | AGUUAAUU | | ACTTTAAGTTAATTTATGA | | |
| | UAU | | TTGATATT | | |
| IL6_947 | UAUAGUUU | 1447 | TGAAATGTTATATGTTATA | 1647 | mouse |
| | UGAAAUGA | | GTTTTGAAATGATAACCT | | |
| | UAAC | | AAAAATCT | | |
| IL6_649 | CAAUUCAUC | 1448 | AGGACCAAGACCATCCAA | 1648 | mouse |
| | UUGAAAUCA | | TTCATCTTGAAATCACTTG | | |
| | CU | | AAGAATTT | | |
| IL6_897 | AUGAUUGA | 1449 | ACTTTAAGTTAATTTATGA | 1649 | mouse |
| | UAUUUAUU | | TTGATATTTATTATTTTTA | | |
| | AUUU | | TGAAGTG | | |
| IL6_1043 | AUUGCUAAU | 1450 | AGTTTACCTCAATGAATTG | 1650 | mouse |
| | UUAAAUAU | | CTAATTTAAATATGTTTTT | | |
| | GUU | | AAAGAAA | | |
| IL6_576 | UACCCCAAU | 1451 | TAAAATAGTCCTTCCTACC | 1651 | mouse |
| | UUCCAAUGC | | CCAATTTCCAATGCTCTCC | | |
| | UC | | TAACAGA | | |
| IL6_90 | CUCUGCAAG | 1452 | CGCTATGAAGTTCCTCTCT | 1652 | mouse |
| | AGACUUCCA | | GCAAGAGACTTCCATCCA | | |
| | UC | | GTTGCCTT | | |
| IL6_378 | CCAAACUGG | 1453 | AAATGATGGATGCTACCA | 1653 | mouse |
| | AUAUAAUCA | | AACTGGATATAATCAGGA | | |
| | GG | | AATTTGCCT | | |
| IL6_853 | UUUAAUUU | 1454 | ACTATTTTAATTATTTTTA | 1654 | mouse |
| | AUUGAUAA | | ATTTATTGATAATTTAAAT | | |
| | UUUA | | AAGTAAA | | |
| IL6_367 | GAUGGAUGC | 1455 | GAGATACAAAGAAATGAT | 1655 | mouse |
| | UACCAAACU | | GGATGCTACCAAACTGGA | | |
| | GG | | TATAATCAG | | |
| IL6_992 | UCUGUUACC | 1456 | ATTTGATATAAATATTCTG | 1656 | mouse |
| | UAGCCAGAU | | TTACCTAGCCAGATGGTTT | | |
| | GG | | CTTGGAA | | |
| IL6_939 | UUAUAUGU | 1457 | GTGTCACTTGAAATGTTAT | 1657 | mouse |
| | UAUAGUUU | | ATGTTATAGTTTTGAAATG | | |
| | UGAA | | ATAACCT | | |
| IL6_1037 | CAAUGAAUU | 1458 | TGTATAAGTTTACCTCAAT | 1658 | mouse |
| | GCUAAUUUA | | GAATTGCTAATTTAAATAT | | |
| | AA | | GTTTTTA | | |
| IL6_661 | AAAUCACUU | 1459 | ATCCAATTCATCTTGAAAT | 1659 | mouse |
| | GAAGAAUU | | CACTTGAAGAATTTCTAA | | |
| | UCU | | AAGTCACT | | |
| IL6_1042 | AAUUGCUAA | 1460 | AAGTTTACCTCAATGAATT | 1660 | mouse |
| | UUUAAAUA | | GCTAATTTAAATATGTTTT | | |
| | UGU | | TAAAGAA | | |
| IL6_995 | GUUACCUAG | 1461 | TGATATAAATATTCTGTTA | 1661 | mouse |
| | CCAGAUGGU | | CCTAGCCAGATGGTTTCTT | | |
| | UU | | GGAATGT | | |
| IL6_1017 | UGGAAUGU | 1462 | AGCCAGATGGTTTCTTGG | 1662 | mouse |
| | AUAAGUUU | | AATGTATAAGTTTACCTCA | | |
| | ACCU | | ATGAATTG | | |
| IL6_929 | ACUUGAAAU | 1463 | TTTTTATGAAGTGTCACTT | 1663 | mouse |
| | GUUAUAUG | | GAAATGTTATATGTTATA | | |
| | UUA | | GTTTTGAA | | |
| IL6_878 | GUAAACUUU | 1464 | TGATAATTTAAATAAGTA | 1664 | mouse |
| | AAGUUAAU | | AACTTTAAGTTAATTTATG | | |
| | UUA | | ATTGATAT | | |
| IL6_945 | GUUAUAGU | 1465 | CTTGAAATGTTATATGTTA | 1665 | mouse |
| | UUUGAAAU | | TAGTTTTGAAATGATAAC | | |
| | GAUA | | CTAAAAAT | | |
| IL6_285 | GUGCAAUGG | 1466 | AATGAGAAAAGAGTTGTG | 1666 | mouse |
| | CAAUUCUGA | | CAATGGCAATTCTGATTGT | | |
| | UU | | ATGAACAA | | |
| IL6_662 | AAUCACUUG | 1467 | TCCAATTCATCTTGAAATC | 1667 | mouse |
| | AAGAAUUUC | | ACTTGAAGAATTTCTAAA | | |
| | UA | | AGTCACTT | | |
| IL6_852 | UUUUAAUU | 1468 | CACTATTTTAATTATTTTT | 1668 | mouse |
| | UAUUGAUA | | AATTTATTGATAATTTAAA | | |
| | AUUU | | TAAGTAA | | |
| IL6_348 | UCCAGAGAU | 1469 | AAACAATCTGAAACTTCC | 1669 | mouse |
| | ACAAAGAAA | | AGAGATACAAAGAAATGA | | |
| | UG | | TGGATGCTA | | |
| IL6_883 | CUUUAAGUU | 1470 | ATTTAAATAAGTAAACTTT | 1670 | mouse |
| | AAUUUAUG | | AAGTTAATTTATGATTGAT | | |
| | AUU | | ATTTATT | | |
| IL6_1033 | ACCUCAAUG | 1471 | GGAATGTATAAGTTTACC | 1671 | mouse |
| | AAUUGCUAA | | TCAATGAATTGCTAATTTA | | |
| | UU | | AATATGTT | | |
| IL6_758 | UGGUCAGAA | 1472 | GGACATTCCTCACTGTGGT | 1672 | mouse |
| | AAUAUAUCC | | CAGAAAATATATCCTGTT | | |
| | UG | | GTCAGGTA | | |
| IL6_1090 | UAUAAUGU | 1473 | TTTGTGATGTATTTTTATA | 1673 | mouse |
| | UUAGACUGU | | ATGTTTAGACTGTCTTCAA | | |
| | CUU | | ACAAATA | | |
| IL6_979 | UUGAUAUA | 1474 | AACCTAAAAATCTATTTG | 1674 | mouse |
| | AAUAUUCUG | | ATATAAATATTCTGTTACC | | |
| | UUA | | TAGCCAGA | | |
| IL6_916 | UUUAUGAA | 1475 | TTGATATTTATTATTTTTA | 1675 | mouse |
| | GUGUCACUU | | TGAAGTGTCACTTGAAAT | | |
| | GAA | | GTTATATG | | |
| IL6_328 | GCAGAAAAC | 1476 | AACGATGATGCACTTGCA | 1676 | mouse |
| | AAUCUGAAA | | GAAAACAATCTGAAACTT | | |
| | CU | | CCAGAGATA | | |
| IL6_831 | UUGGGACAC | 1477 | CTGACAATATGAATGTTG | 1677 | mouse |
| | UAUUUUAA | | GGACACTATTTTAATTATT | | |
| | UUA | | TTTAATTT | | |
| IL6_385 | GGAUAUAA | 1478 | GGATGCTACCAAACTGGA | 1678 | mouse |
| | UCAGGAAAU | | TATAATCAGGAAATTTGC | | |
| | UUG | | CTATTGAAA | | |
| IL6_1087 | UUUUAUAA | 1479 | ATCTTTGTGATGTATTTTT | 1679 | mouse |
| | UGUUUAGAC | | ATAATGTTTAGACTGTCTT | | |
| | UGU | | CAAACAA | | |
| IL6_249 | UGUUCUCUG | 1480 | AGGCTTAATTACACATGTT | 1680 | mouse |
| | GGAAAUCGU | | CTCTGGGAAATCGTGGAA | | |
| | GG | | ATGAGAAA | | |
| IL6_670 | GAAGAAUU | 1481 | ATCTTGAAATCACTTGAA | 1681 | mouse |
| | UCUAAAAGU | | GAATTTCTAAAAGTCACTT | | |
| | CAC | | TGAGATCT | | |
| IL6_653 | UCAUCUUGA | 1482 | CCAAGACCATCCAATTCA | 1682 | mouse |
| | AAUCACUUG | | TCTTGAAATCACTTGAAG | | |
| | AA | | AATTTCTAA | | |
| IL6_283 | UUGUGCAAU | 1483 | GAAATGAGAAAAGAGTTG | 1683 | mouse |
| | GGCAAUUCU | | TGCAATGGCAATTCTGATT | | |
| | GA | | GTATGAAC | | |
| IL6_532 | AUCUUCAAC | 1484 | GAAACTCTAATTCATATCT | 1684 | mouse |
| | CAAGAGGUA | | TCAACCAAGAGGTAAAAG | | |
| | AA | | ATTTACAT | | |
| IL6_996 | UUACCUAGC | 1485 | GATATAAATATTCTGTTAC | 1685 | mouse |
| | CAGAUGGUU | | CTAGCCAGATGGTTTCTTG | | |
| | UC | | GAATGTA | | |
| IL6_1032 | UACCUCAAU | 1486 | TGGAATGTATAAGTTTAC | 1686 | mouse |
| | GAAUUGCUA | | CTCAATGAATTGCTAATTT | | |
| | AU | | AAATATGT | | |
| IL6_657 | CUUGAAAUC | 1487 | GACCATCCAATTCATCTTG | 1687 | mouse |
| | ACUUGAAGA | | AAATCACTTGAAGAATTT | | |
| | AU | | CTAAAAGT | | |
| IL6_895 | UUAUGAUU | 1488 | AAACTTTAAGTTAATTTAT | 1688 | mouse |
| | GAUAUUUA | | GATTGATATTTATTATTTT | | |
| | UUAU | | TATGAAG | | |
| IL6_400 | AUUUGCCUA | 1489 | GGATATAATCAGGAAATT | 1689 | mouse |
| | UUGAAAAU | | TGCCTATTGAAAATTTCCT | | |
| | UUC | | CTGGTCTT | | |
| IL6_1014 | UCUUGGAAU | 1490 | CCTAGCCAGATGGTTTCTT | 1690 | mouse |
| | GUAUAAGU | | GGAATGTATAAGTTTACC | | |
| | UUA | | TCAATGAA | | |
| IL6_866 | UAAUUUAA | 1491 | TTTTTAATTTATTGATAAT | 1691 | mouse |
| | AUAAGUAA | | TTAAATAAGTAAACTTTA | | |
| | ACUU | | AGTTAATT | | |
| IL6_339 | UCUGAAACU | 1492 | ACTTGCAGAAAACAATCT | 1692 | mouse |
| | UCCAGAGAU | | GAAACTTCCAGAGATACA | | |
| | AC | | AAGAAATGA | | |
| IL6_233 | GAGGCUUAA | 1493 | CCACTTCACAAGTCGGAG | 1693 | mouse |
| | UUACACAUG | | GCTTAATTACACATGTTCT | | |
| | UU | | CTGGGAAA | | |
| IL6_877 | AGUAAACUU | 1494 | TTGATAATTTAAATAAGT | 1694 | mouse |
| | UAAGUUAA | | AAACTTTAAGTTAATTTAT | | |
| | UUU | | GATTGATA | | |

TABLE 11C
Host target FURIN - 20 nucleotide targets and 45 nucleotide gene
target regions

| | | SEQ | | SEQ | |
|---|---|---|---|---|---|
| SEQUENCE | | ID | | ID | |
| ID | 20 nt Sequence | NO: | 45 nt Gene Region | NO: | Species |
| FURIN_4183 | AUGGACAUGAG | 1695 | TGCTGGTTCTATTTAATGGACAT | 1795 | human |
| | AUAAUGUUA | | GAGATAATGTTAGAGGTTTTAA | | |
| FURIN_3561 | UUUAGAUGCUG | 1696 | TTGTGATTATTTCACTTTAGATG | 1796 | human |
| | AUGAUUUGU | | CTGATGATTTGTTTTTGTATTT | | |
| FURIN_3555 | UUUCACUUUAG | 1697 | TTCACTTTGTGATTATTTCACTTT | 1797 | human |
| | AUGCUGAUG | | AGATGCTGATGATTTGTTTTT | | |
| FURIN_3563 | UAGAUGCUGAU | 1698 | GTGATTATTTCACTTTAGATGCT | 1798 | human |
| | GAUUUGUUU | | GATGATTTGTTTTTGTATTTTT | | |
| FURIN_3529 | GAGGAUAUAUU | 1699 | TCTCAGGGGCTGTTTGAGGATAT | 1799 | human |
| | UUCACUUUG | | ATTTTCACTTTGTGATTATTTC | | |
| FURIN_4161 | CCAGCAUUGCU | 1700 | GTAATTTAAACAGGCCCAGCATT | 1800 | human |
| | GGUUCUAUU | | GCTGGTTCTATTTAATGGACAT | | |
| FURIN_3537 | AUUUUCACUUU | 1701 | GCTGTTTGAGGATATATTTTCAC | 1801 | human |
| | GUGAUUAUU | | TTTGTGATTATTTCACTTTAGA | | |
| FURIN_1873 | GCCUUCAUGAC | 1702 | GGGTTTAATGACTGGGCCTTCAT | 1802 | human |
| | AACUCAUUC | | GACAACTCATTCCTGGGATGAG | | |
| FURIN_3545 | UUUGUGAUUAU | 1703 | AGGATATATTTTCACTTTGTGAT | 1803 | human |
| | UUCACUUUA | | TATTTCACTTTAGATGCTGATG | | |
| FURIN_3525 | GUUUGAGGAUA | 1704 | GGGATCTCAGGGGCTGTTTGAGG | 1804 | human |
| | UAUUUUCAC | | ATATATTTTCACTTTGTGATTA | | |
| FURIN_4184 | UGGACAUGAGA | 1705 | GCTGGTTCTATTTAATGGACATG | 1805 | human |
| | UAAUGUUAG | | AGATAATGTTAGAGGTTTTAAA | | |
| FURIN_3995 | UCUGGGAGUCC | 1706 | ATCAGTCCCCTCCCATCTGGGAG | 1806 | human |
| | CCUUUUCUU | | TCCCCTTTTCTTTTCTACCCTA | | |
| FURIN_4152 | UAAACAGGCCC | 1707 | TTTTTTCTTGTAATTTAAACAGG | 1807 | human |
| | AGCAUUGCU | | CCCAGCATTGCTGGTTCTATTT | | |
| FURIN_1878 | CAUGACAACUC | 1708 | TAATGACTGGGCCTTCATGACAA | 1808 | human |
| | AUUCCUGGG | | CTCATTCCTGGGATGAGGATCC | | |
| FURIN_4192 | AGAUAAUGUUA | 1709 | TATTTAATGGACATGAGATAATG | 1809 | human |
| | GAGGUUUUA | | TTAGAGGTTTTAAAGTGATTAA | | |
| FURIN_1969 | ACCAAGUUCAC | 1710 | AACTATGGGACGCTGACCAAGTT | 1810 | human |
| | CCUCGUACU | | CACCCTCGTACTCTATGGCACC | | |
| FURIN_312 | AGCAGCAACAG | 1711 | GTTGCTATGGGTGGTAGCAGCAA | 1811 | human |
| | GAACCUUGG | | CAGGAACCTTGGTCCTGCTAGC | | |
| FURIN_3538 | UUUUCACUUUG | 1712 | CTGTTTGAGGATATATTTTCACT | 1812 | human |
| | UGAUUAUUU | | TTGTGATTATTTCACTTTAGAT | | |
| FURIN_4193 | GAUAAUGUUAG | 1713 | ATTTAATGGACATGAGATAATGT | 1813 | human |
| | AGGUUUUAA | | TAGAGGTTTTAAAGTGATTAAA | | |
| FURIN_3957 | UUUGCACCCCU | 1714 | TCTTCTGACGTGCCTTTTGCACC | 1814 | human |
| | CCCAUUAGG | | CCTCCCATTAGGACAATCAGTC | | |
| FURIN_1925 | UAGAGAUUGAA | 1715 | CTGGCGAGTGGGTCCTAGAGATT | 1815 | human |
| | AACACCAGC | | GAAAACACCAGCGAAGCCAACA | | |
| FURIN_2894 | AAAGGAGUGAA | 1716 | CCCTTCCATGTGGAGAAAGGAGT | 1816 | human |
| | ACCUUUAGG | | GAAACCTTTAGGGCAGCTTGCC | | |
| FURIN_4117 | CUGGGUUGGUG | 1717 | TGGTTTTGTAAGATGCTGGGTTG | 1817 | human |
| | CACAGUGAU | | GTGCACAGTGATTTTTTTCTTG | | |
| FURIN_3534 | UAUAUUUUCAC | 1718 | GGGGCTGTTTGAGGATATATTTT | 1818 | human |
| | UUUGUGAUU | | CACTTTGTGATTATTTCACTTT | | |
| FURIN_4150 | UUUAAACAGGC | 1719 | ATTTTTTTCTTGTAATTTAAACAG | 1819 | human |
| | CCAGCAUUG | | GCCCAGCATTGCTGGTTCTAT | | |
| FURIN_3929 | CCCUCAAACCU | 1720 | TGACCTGTCATGCCCCCCTCAAA | 1820 | human |
| | CCUCUUCUG | | CCTCCTCTTCTGACGTGCCTTT | | |
| FURIN_3560 | CUUUAGAUGCU | 1721 | TTTGTGATTATTTCACTTTAGATG | 1821 | human |
| | GAUGAUUUG | | CTGATGATTTGTTTTTGTATT | | |
| FURIN_4100 | GCUGGUUUUGU | 1722 | CTCGTGGCCAGCCCGGCTGGTTT | 1822 | human |
| | AAGAUGCUG | | TGTAAGATGCTGGGTTGGTGCA | | |
| FURIN_712 | CACGGCAUUGU | 1723 | CAGGGCTACACAGGGCACGGCA | 1823 | human |
| | GGUCUCCAU | | TTGTGGTCTCCATTCTGGACGAT | | |
| FURIN_3541 | UCACUUUGUGA | 1724 | TTTGAGGATATATTTTCACTTTGT | 1824 | human |
| | UUAUUUCAC | | GATTATTTCACTTTAGATGCT | | |
| FURIN_4169 | GCUGGUUCUAU | 1725 | AACAGGCCCAGCATTGCTGGTTC | 1825 | human |
| | UUAAUGGAC | | TATTTAATGGACATGAGATAAT | | |
| FURIN_1303 | AGCAGUGGCAA | 1726 | CTGGCCACGACCTACAGCAGTG | 1826 | human |
| | CCAGAAUGA | | GCAACCAGAATGAGAAGCAGATC | | |
| FURIN_765 | CUUGGCAGGCA | 1727 | GAAGAACCACCCGGACTTGGCA | 1827 | human |
| | AUUAUGAUC | | GGCAATTATGATCCTGGGGCCAG | | |
| FURIN_1942 | AGCGAAGCCAA | 1728 | GAGATTGAAAACACCAGCGAAG | 1828 | human |
| | CAACUAUGG | | CCAACAACTATGGGACGCTGACC | | |
| FURIN_1945 | GAAGCCAACAA | 1729 | ATTGAAAACACCAGCGAAGCCA | 1829 | human |
| | CUAUGGGAC | | ACAACTATGGGACGCTGACCAAG | | |
| FURIN_2780 | GGAGACUGCUU | 1730 | GGAGGCAAGAGGGGTGGAGACT | 1830 | human |
| | CCCAUCCUA | | GCTTCCCATCCTACCCTCGGGCC | | |
| FURIN_3827 | UCAUAGGUCAC | 1731 | CGCCATGCCGGGGGTTCATAGGT | 1831 | human |
| | UGGCUCUCC | | CACTGGCTCTCCAAGTGCCAGA | | |
| FURIN_3547 | UGUGAUUAUUU | 1732 | GATATATTTTCACTTTGTGATTAT | 1832 | human |
| | CACUUUAGA | | TTCACTTTAGATGCTGATGAT | | |
| FURIN_3559 | ACUUUAGAUGC | 1733 | CTTTGTGATTATTTCACTTTAGAT | 1833 | human |
| | UGAUGAUUU | | GCTGATGATTTGTTTTTGTAT | | |
| FURIN_4177 | UAUUUAAUGGA | 1734 | CAGCATTGCTGGTTCTATTTAAT | 1834 | human |
| | CAUGAGAUA | | GGACATGAGATAATGTTAGAGG | | |
| FURIN_2158 | UAUAGCACCGA | 1735 | GTCCTCGATACGCACTATAGCAC | 1835 | human |
| | GAAUGACGU | | CGAGAATGACGTGGAGACCATC | | |
| FURIN_4165 | CAUUGCUGGUU | 1736 | TTTAAACAGGCCCAGCATTGCTG | 1836 | human |
| | CUAUUUAAU | | GTTCTATTTAATGGACATGAGA | | |
| FURIN_4186 | GACAUGAGAUA | 1737 | TGGTTCTATTTAATGGACATGAG | 1837 | human |
| | AUGUUAGAG | | ATAATGTTAGAGGTTTTAAAGT | | |
| FURIN_779 | AUGAUCCUGGG | 1738 | ACTTGGCAGGCAATTATGATCCT | 1838 | human |
| | GCCAGUUUU | | GGGGCCAGTTTTGATGTCAATG | | |
| FURIN_1871 | GGGCCUUCAUG | 1739 | ATGGGTTTAATGACTGGGCCTTC | 1839 | human |
| | ACAACUCAU | | ATGACAACTCATTCCTGGGATG | | |
| FURIN_3552 | UUAUUUCACUU | 1740 | ATTTTCACTTTGTGATTATTTCAC | 1840 | human |
| | UAGAUGCUG | | TTTAGATGCTGATGATTTGTT | | |
| FURIN_4180 | UUAAUGGACAU | 1741 | CATTGCTGGTTCTATTTAATGGA | 1841 | human |
| | GAGAUAAUG | | CATGAGATAATGTTAGAGGTTT | | |
| FURIN_1429 | GCCAAUAAGAA | 1742 | GCTCTCACCCTGGAGGCCAATAA | 1842 | human |
| | CCUCACAUG | | GAACCTCACATGGCGGGACATG | | |
| FURIN_1654 | AAAGACAUCGG | 1743 | ATCCTCACCGAGCCCAAAGACAT | 1843 | human |
| | GAAACGGCU | | CGGGAAACGGCTCGAGGTGCGG | | |
| FURIN_3968 | CCCAUUAGGAC | 1744 | GCCTTTTGCACCCCTCCCATTAG | 1844 | human |
| | AAUCAGUCC | | GACAATCAGTCCCCTCCCATCT | | |
| FURIN_1855 | GAUGGGUUUAA | 1745 | CATGACTACTCCGCAGATGGGTT | 1845 | human |
| | UGACUGGGC | | TAATGACTGGGCCTTCATGACA | | |
| FURIN_3513 | AUCUCAGGGGC | 1746 | CTTTCCCCTGTGGGGATCTCAGG | 1846 | human |
| | UGUUUGAGG | | GGCTGTTTGAGGATATATTTTC | | |
| FURIN_4108 | UGUAAGAUGCU | 1747 | CAGCCCGGCTGGTTTTGTAAGAT | 1847 | human |
| | GGGUUGGUG | | GCTGGGTTGGTGCACAGTGATT | | |
| FURIN_4170 | CUGGUUCUAUU | 1748 | ACAGGCCCAGCATTGCTGGTTCT | 1848 | human |
| | UAAUGGACA | | ATTTAATGGACATGAGATAATG | | |
| FURIN_367 | UUCACCAACAC | 1749 | CAGGGCCAGAAGGTCTTCACCA | 1849 | human |
| | GUGGGCUGU | | ACACGTGGGCTGTGCGCATCCCT | | |
| FURIN_2492 | UGCGCUCUGGC | 1750 | TCCTGGTCCTGCAGCTGCGCTCT | 1850 | human |
| | UUUAGUUUU | | GGCTTTAGTTTTCGGGGGGTGA | | |
| FURIN_2882 | UUCCAUGUGGA | 1751 | CACCCTCAGCACCCCTTCCATGT | 1851 | human |
| | GAAAGGAGU | | GGAGAAAGGAGTGAAACCTTTA | | |
| FURIN_4175 | UCUAUUUAAUG | 1752 | CCCAGCATTGCTGGTTCTATTTA | 1852 | human |
| | GACAUGAGA | | ATGGACATGAGATAATGTTAGA | | |
| FURIN_4201 | UAGAGGUUUUA | 1753 | GACATGAGATAATGTTAGAGGTT | 1853 | human |
| | AAGUGAUUA | | TTAAAGTGATTAAACGTGCAGA | | |
| FURIN_749 | AGAAGAACCAC | 1754 | TGGACGATGGCATCGAGAAGAA | 1854 | human |
| | CCGGACUUG | | CCACCCGGACTTGGCAGGCAATT | | |
| FURIN_3523 | CUGUUUGAGGA | 1755 | TGGGGATCTCAGGGGCTGTTTGA | 1855 | human |
| | UAUAUUUUC | | GGATATATTTTCACTTTGTGAT | | |
| FURIN_1856 | AUGGGUUUAAU | 1756 | ATGACTACTCCGCAGATGGGTTT | 1856 | human |
| | GACUGGGCC | | AATGACTGGGCCTTCATGACAA | | |
| FURIN_1859 | GGUUUAAUGAC | 1757 | ACTACTCCGCAGATGGGTTTAAT | 1857 | human |
| | UGGGCCUUC | | GACTGGGCCTTCATGACAACTC | | |
| FURIN_2638 | UUUAUCAAAGA | 1758 | GGCGAGAGGACCGCCTTTATCA | 1858 | human |
| | CCAGAGCGC | | AAGACCAGAGCGCCCTCTGATGA | | |
| FURIN_3516 | UCAGGGGCUGU | 1759 | TCCCCTGTGGGGATCTCAGGGGC | 1859 | human |
| | UUGAGGAUA | | TGTTTGAGGATATATTTTCACT | | |
| FURIN_3554 | AUUUCACUUUA | 1760 | TTTCACTTTGTGATTATTTCACTT | 1860 | human |
| | GAUGCUGAU | | TAGATGCTGATGATTTGTTTT | | |
| FURIN_1936 | AACACCAGCGA | 1761 | GTCCTAGAGATTGAAAACACCA | 1861 | human |
| | AGCCAACAA | | GCGAAGCCAACAACTATGGGACG | | |
| FURIN_2458 | CUGGUCUUCGU | 1762 | TGCGCCTTCATCGTGCTGGTCTT | 1862 | human |
| | CACUGUCUU | | CGTCACTGTCTTCCTGGTCCTG | | |
| FURIN_313 | GCAGCAACAGG | 1763 | TTGCTATGGGTGGTAGCAGCAAC | 1863 | human |
| | AACCUUGGU | | AGGAACCTTGGTCCTGCTAGCA | | |
| FURIN_2520 | GAAGGUGUACA | 1764 | TAGTTTTCGGGGGGTGAAGGTGT | 1864 | human |
| | CCAUGGACC | | ACACCATGGACCGTGGCCTCAT | | |
| FURIN_1310 | GCAACCAGAAU | 1765 | CGACCTACAGCAGTGGCAACCA | 1865 | human |
| | GAGAAGCAG | | GAATGAGAAGCAGATCGTGACGA | | |
| FURIN_1752 | CACCCUGUCCU | 1766 | CGCTCAGGCGCGGCTCACCCTGT | 1866 | human |
| | AUAAUCGCC | | CCTATAATCGCCGTGGCGACCT | | |
| FURIN_4166 | AUUGCUGGUUC | 1767 | TTAAACAGGCCCAGCATTGCTGG | 1867 | human |
| | UAUUUAAUG | | TTCTATTTAATGGACATGAGAT | | |
| FURIN_834 | GUACACACAGA | 1768 | TGACCCCCAGCCTCGGTACACAC | 1868 | human |
| | UGAAUGACA | | AGATGAATGACAACAGGCACGG | | |
| FURIN_3517 | CAGGGGCUGUU | 1769 | CCCCTGTGGGGATCTCAGGGGCT | 1869 | human |
| | UGAGGAUAU | | GTTTGAGGATATATTTTCACTT | | |
| FURIN_3550 | GAUUAUUUCAC | 1770 | ATATTTTCACTTTGTGATTATTTC | 1870 | human |
| | UUUAGAUGC | | ACTTTAGATGCTGATGATTTG | | |
| FURIN_2694 | UCCCCUCCUUG | 1771 | ACCCCCTCAAGCCAATCCCCTCC | 1871 | human |
| | GGCACUUUU | | TTGGGCACTTTTTAATTCACCA | | |
| FURIN_578 | UGGCAAAGCGA | 1772 | GGCTGGAACAGCAGGTGGCAAA | 1872 | human |
| | CGGACUAAA | | GCGACGGACTAAACGGGACGTGT | | |
| FURIN_1246 | UUUGGCAACGU | 1773 | AGCAGCGCCACGCAGTTTGGCA | 1873 | human |
| | GCCGUGGUA | | ACGTGCCGTGGTACAGCGAGGCC | | |
| FURIN_1425 | GGAGGCCAAUA | 1774 | CATTGCTCTCACCCTGGAGGCCA | 1874 | human |
| | AGAACCUCA | | ATAAGAACCTCACATGGCGGGA | | |
| FURIN_1018 | AUCCACAUCUA | 1775 | CTGAACCCCAACCACATCCACAT | 1875 | human |
| | CAGUGCCAG | | CTACAGTGCCAGCTGGGGCCCC | | |
| FURIN_1858 | GGGUUUAAUGA | 1776 | GACTACTCCGCAGATGGGTTTAA | 1876 | human |
| | CUGGGCCUU | | TGACTGGGCCTTCATGACAACT | | |
| FURIN_1924 | CUAGAGAUUGA | 1777 | TCTGGCGAGTGGGTCCTAGAGAT | 1877 | human |
| | AAACACCAG | | TGAAAACACCAGCGAAGCCAAC | | |
| FURIN_293 | CCUGGUUGCUA | 1778 | CCATGGAGCTGAGGCCCTGGTTG | 1878 | human |
| | UGGGUGGUA | | CTATGGGTGGTAGCAGCAACAG | | |
| FURIN_790 | GCCAGUUUUGA | 1779 | AATTATGATCCTGGGGCCAGTTT | 1879 | human |
| | UGUCAAUGA | | TGATGTCAATGACCAGGACCCT | | |
| FURIN_962 | AUGGCGAGGUG | 1780 | GGGTGCGCATGCTGGATGGCGA | 1880 | human |
| | ACAGAUGCA | | GGTGACAGATGCAGTGGAGGCAC | | |
| FURIN_1757 | UGUCCUAUAAU | 1781 | AGGCGCGGCTCACCCTGTCCTAT | 1881 | human |
| | CGCCGUGGC | | AATCGCCGTGGCGACCTGGCCA | | |
| FURIN_1848 | CUCCGCAGAUG | 1782 | CAGGCCACATGACTACTCCGCAG | 1882 | human |
| | GGUUUAAUG | | ATGGGTTTAATGACTGGGCCTT | | |
| FURIN_3020 | GUCCCUCUAAA | 1783 | CTCTTGCCCTTCCCTGTCCCTCTA | 1883 | human |
| | GCAAUAAUG | | AAGCAATAATGGTCCCATCCA | | |
| FURIN_3511 | GGAUCUCAGGG | 1784 | TGCTTTCCCCTGTGGGGATCTCA | 1884 | human |
| | GCUGUUUGA | | GGGGCTGTTTGAGGATATATTT | | |
| FURIN_760 | CCGGACUUGGC | 1785 | ATCGAGAAGAACCACCCGGACT | 1885 | human |
| | AGGCAAUUA | | TGGCAGGCAATTATGATCCTGGG | | |
| FURIN_770 | CAGGCAAUUAU | 1786 | ACCACCCGGACTTGGCAGGCAA | 1886 | human |
| | GAUCCUGGG | | TTATGATCCTGGGGCCAGTTTTG | | |
| FURIN_827 | AGCCUCGGUAC | 1787 | AGGACCCTGACCCCCAGCCTCGG | 1887 | human |
| | ACACAGAUG | | TACACACAGATGAATGACAACA | | |
| FURIN_1199 | GCUACACCAAC | 1788 | GCTGCAACTGCGACGGCTACACC | 1888 | human |
| | AGUAUCUAC | | AACAGTATCTACACGCTGTCCA | | |
| FURIN_2459 | UGGUCUUCGUC | 1789 | GCGCCTTCATCGTGCTGGTCTTC | 1889 | human |
| | ACUGUCUUC | | GTCACTGTCTTCCTGGTCCTGC | | |
| FURIN_4106 | UUUGUAAGAUG | 1790 | GCCAGCCCGGCTGGTTTTGTAAG | 1890 | human |
| | CUGGGUUGG | | ATGCTGGGTTGGTGCACAGTGA | | |
| FURIN_4173 | GUUCUAUUUAA | 1791 | GGCCCAGCATTGCTGGTTCTATT | 1891 | human |
| | UGGACAUGA | | TAATGGACATGAGATAATGTTA | | |
| FURIN_4199 | GUUAGAGGUUU | 1792 | TGGACATGAGATAATGTTAGAG | 1892 | human |
| | UAAAGUGAU | | GTTTTAAAGTGATTAAACGTGCA | | |
| FURIN_773 | GCAAUUAUGAU | 1793 | ACCCGGACTTGGCAGGCAATTAT | 1893 | human |
| | CCUGGGGCC | | GATCCTGGGGCCAGTTTTGATG | | |
| FURIN_786 | UGGGGCCAGUU | 1794 | AGGCAATTATGATCCTGGGGCCA | 1894 | human |
| | UUGAUGUCA | | GTTTTGATGTCAATGACCAGGA | | |

TABLE 11D
Host target ACE2-20 nucleotide targets and 45 nucleotide gene target regions

| SE- | | SEQ | | SEQ | |
|---|---|---|---|---|---|
| QUENCE | | ID | | ID | |
| ID | 20 nt Sequence | NO: | 45 nt Gene Region | NO: | Species |
| ACE2_ | UUGGAUUUCAUAC | 1895 | TGACATAGATACTCTTTGGATTT | 2095 | human |
| 52 | CAUGUGG | | CATACCATGTGGAGGCTTTCTT | | |
| ACE2_ | UGUCAAAACUAUG | 1896 | AAAGATATCATTAAATGTCAAA | 2096 | human |
| 2730 | ACUCUGU | | ACTATGACTCTGTTCAGAAAAAA | | |
| ACE2_ | CCUAGCAUUGGAA | 1897 | ATCAGAACCCTGGACCCTAGCAT | 2097 | human |
| 1918 | AAUGUUG | | TGGAAAATGTTGTAGGAGCAAA | | |
| ACE2_ | AUGUAAAUGUUAA | 1898 | AGGTGATTTTGTTGTATGTAAAT | 2098 | human |
| 2674 | UUUCAUG | | GTTAATTTCATGGTATAGAAAA | | |
| ACE2_ | UCUGUUUCUUAAU | 1899 | GGATTTGACTTCTGTTCTGTTTCT | 2099 | human |
| 2849 | AAGGAUU | | TAATAAGGATTTTGTATTAGA | | |
| ACE2_ | CAGGAGUUGACAU | 1900 | ATGGCTACAGAGGATCAGGAGT | 2100 | human |
| 30 | AGAUACU | | TGACATAGATACTCTTTGGATTT | | |
| ACE2_ | AAUGAUUACUCAU | 1901 | CTGTTCCATGTTTCTAATGATTA | 2101 | human |
| 1739 | UCAUUCG | | CTCATTCATTCGATATTACACA | | |
| ACE2_ | AUCGAUAUUAGCA | 1902 | AATCCTTATGCCTCCATCGATAT | 2102 | human |
| 2567 | AAGGAGA | | TAGCAAAGGAGAAAATAATCCA | | |
| ACE2_ | CAUGGUAUAGAAA | 1903 | TGTAAATGTTAATTTCATGGTAT | 2103 | human |
| 2690 | AUAUAAG | | AGAAAATATAAGATGATAAAGA | | |
| ACE2_ | UUUGAAACCAAGA | 1904 | TGTGCGAGTGGCTAATTTGAAAC | 2104 | human |
| 2239 | AUCUCCU | | CAAGAATCTCCTTTAATTTCTT | | |
| ACE2_ | AUGGGAGUGAUAG | 1905 | GTTTTTGGAGTTGTGATGGGAGT | 2105 | human |
| 2465 | UGGUUGG | | GATAGTGGTTGGCATTGTCATC | | |
| ACE2_ | UUGAAGAGAUUAA | 1906 | ATGTGGAACATACCTTTGAAGAG | 2106 | human |
| 906 | ACCAUUA | | ATTAAACCATTATATGAACATC | | |
| ACE2_ | UUUGUUGUAUGUA | 1907 | TCCTCTTGAGGTGATTTTGTTGT | 2107 | human |
| 2666 | AAUGUUA | | ATGTAAATGTTAATTTCATGGT | | |
| ACE2_ | UUCAUGGUAUAGA | 1908 | TATGTAAATGTTAATTTCATGGT | 2108 | human |
| 2688 | AAAUAUA | | ATAGAAAATATAAGATGATAAA | | |
| ACE2_ | CUCUGGAUUUGAC | 1909 | AGTATTTATTTCTGTCTCTGGATT | 2109 | human |
| 2830 | UUCUGUU | | TGACTTCTGTTCTGTTTCTTA | | |
| ACE2_ | UAAACCAUUAUAU | 1910 | TACCTTTGAAGAGATTAAACCAT | 2110 | human |
| 916 | GAACAUC | | TATATGAACATCTTCATGCCTA | | |
| ACE2_ | UUCCUCUUGAGGU | 1911 | GAAAAATCTATGTTTTTCCTCTT | 2111 | human |
| 2650 | GAUUUUG | | GAGGTGATTTTGTTGTATGTAA | | |
| ACE2_ | CAUUAAAUGUCAA | 1912 | AGATGATAAAGATATCATTAAAT | 2112 | human |
| 2723 | AACUAUG | | GTCAAAACTATGACTCTGTTCA | | |
| ACE2_ | GGAGAAAAUAAUC | 1913 | ATCGATATTAGCAAAGGAGAAA | 2113 | human |
| 2582 | CAGGAUU | | ATAATCCAGGATTCCAAAACACT | | |
| ACE2_ | UGGCCAAGGAGAG | 1914 | TGTCCAAAGACAACATGGCCAA | 2114 | human |
| 2778 | AGCAUCU | | GGAGAGAGCATCTTCATTGACAT | | |
| ACE2_ | UCAGGGAUAAUCU | 1915 | GGTCTCACAGGCTGTTCAGGGAT | 2115 | human |
| 2918 | AAAUGUA | | AATCTAAATGTAAATGTCTGTT | | |
| ACE2_ | UUCAAGAAGACAA | 1916 | TTCTGTCACCCGATTTTCAAGAA | 2116 | human |
| 1500 | UGAAACA | | GACAATGAAACAGAAATAAACT | | |
| ACE2_ | AGGACCCUUUACC | 1917 | ATTCGATATTACACAAGGACCCT | 2117 | human |
| 1769 | AAUUCCA | | TTACCAATTCCAGTTTCAAGAA | | |
| ACE2_ | UGUAGGAGCAAAG | 1918 | AGCATTGGAAAATGTTGTAGGA | 2118 | human |
| 1936 | AACAUGA | | GCAAAGAACATGAATGTAAGGCC | | |
| ACE2_ | AGGAGCAAAGAAC | 1919 | ATTGGAAAATGTTGTAGGAGCA | 2119 | human |
| 1939 | AUGAAUG | | AAGAACATGAATGTAAGGCCACT | | |
| ACE2_ | UGGAGAAAAUCCU | 1920 | AAATAAAGCAAGAAGTGGAGAA | 2120 | human |
| 2545 | UAUGCCU | | AATCCTTATGCCTCCATCGATAT | | |
| ACE2_ | GGUGAUUUUGUUG | 1921 | TGTTTTTCCTCTTGAGGTGATTTT | 2121 | human |
| 2660 | UAUGUAA | | GTTGTATGTAAATGTTAATTT | | |
| ACE2_ | UUCUGCAGCCACA | 1922 | GGAAATCATGTCACTTTCTGCAG | 2122 | human |
| 1447 | CCUAAGC | | CCACACCTAAGCATTTAAAATC | | |
| ACE2_ | AAAACUAUGACUC | 1923 | ATATCATTAAATGTCAAAACTAT | 2123 | human |
| 2734 | UGUUCAG | | GACTCTGTTCAGAAAAAAAATT | | |
| ACE2_ | AUCUAAAUGUAAA | 1924 | GGCTGTTCAGGGATAATCTAAAT | 2124 | human |
| 2927 | UGUCUGU | | GTAAATGTCTGTTGAATTTCTG | | |
| ACE2_ | UUAGUCUAGGGAA | 1925 | TCTCATGAGGAGGTTTTAGTCTA | 2125 | human |
| 167 | AGUCAUU | | GGGAAAGTCATTCAGTGGATGT | | |
| ACE2_ | GGAGCAAGUGUUG | 1926 | CAAGGATATATCATTGGAGCAA | 2126 | human |
| 2982 | GAUCUUG | | GTGTTGGATCTTGTATGGAATAT | | |
| ACE2_ | UUUGACUUCUGUU | 1927 | ATTTCTGTCTCTGGATTTGACTTC | 2127 | human |
| 2837 | CUGUUUC | | TGTTCTGTTTCTTAATAAGGA | | |
| ACE2_ | UUUUGUAUUAGAG | 1928 | GTTTCTTAATAAGGATTTTGTAT | 2128 | human |
| 2867 | UAUAUUA | | TAGAGTATATTAGGGAAAGTGT | | |
| ACE2_ | GAGGCCAUUAUAU | 1929 | GGTCGGCAAGCAGCTGAGGCCA | 2129 | human |
| 745 | GAAGAGU | | TTATATGAAGAGTATGTGGTCTT | | |
| ACE2_ | CCAAGAAUCUCCU | 1930 | GTGGCTAATTTGAAACCAAGAAT | 2130 | human |
| 2246 | UUAAUUU | | CTCCTTTAATTTCTTTGTCACT | | |
| ACE2_ | GAUGAUGUUCAGA | 1931 | GGATTCCAAAACACTGATGATGT | 2131 | human |
| 2612 | CCUCCUU | | TCAGACCTCCTTTTAGAAAAAT | | |
| ACE2_ | UCUGGAUUUGACU | 1932 | GTATTTATTTCTGTCTCTGGATTT | 2132 | human |
| 2831 | UCUGUUC | | GACTTCTGTTCTGTTTCTTAA | | |
| ACE2_ | UCCACCAUUGAGG | 1933 | GTAACTGCTGCTCAGTCCACCAT | 2133 | human |
| 272 | AACAGGC | | TGAGGAACAGGCCAAGACATTT | | |
| ACE2_ | UUAUUACUUGAAC | 1934 | AATCCACAAGAATGCTTATTACT | 2134 | human |
| 641 | CAGGUUU | | TGAACCAGGTTTGAATGAAATA | | |
| ACE2_ | UGAACCAGGUUUG | 1935 | AGAATGCTTATTACTTGAACCAG | 2135 | human |
| 649 | AAUGAAA | | GTTTGAATGAAATAATGGCAAA | | |
| ACE2_ | UGUGGGAUGGAGU | 1936 | GAACAAGAATTCTTTTGTGGGAT | 2136 | human |
| 2026 | ACCGACU | | GGAGTACCGACTGGAGTCCATA | | |
| ACE2_ | UGAGGUGAUUUUG | 1937 | CTATGTTTTTCCTCTTGAGGTGAT | 2137 | human |
| 2657 | UUGUAUG | | TTTGTTGTATGTAAATGTTAA | | |
| ACE2_ | UGUAUUUGCUCAC | 1938 | GTCTCTTAAATCTTTTGTATTTGC | 2138 | human |
| 3435 | AGUGUUU | | TCACAGTGTTTGAGCAGTGCT | | |
| ACE2_ | AUUACUUGAACCA | 1939 | TCCACAAGAATGCTTATTACTTG | 2139 | human |
| 643 | GGUUUGA | | AACCAGGTTTGAATGAAATAAT | | |
| ACE2_ | AUGGCAAGAGCAA | 1940 | GTCTTGAAAAATGAGATGGCAA | 2140 | human |
| 785 | AUCAUUA | | GAGCAAATCATTATGAGGACTAT | | |
| ACE2_ | UGGACAGAAACCA | 1941 | TTTGACAGTTCCCTTTGGACAGA | 2141 | human |
| 1072 | AACAUAG | | AACCAAACATAGATGTTACTGA | | |
| ACE2_ | UGUAUUAGAGUAU | 1942 | TCTTAATAAGGATTTTGTATTAG | 2142 | human |
| 2870 | AUUAGGG | | AGTATATTAGGGAAAGTGTGTA | | |
| ACE2_ | AUAUCAUUGGAGC | 1943 | GTTGAAAACAAGGATATATCATT | 2143 | human |
| 2974 | AAGUGUU | | GGAGCAAGTGTTGGATCTTGTA | | |
| ACE2_ | GUAAAUGUUAAUU | 1944 | GTGATTTTGTTGTATGTAAATGT | 2144 | human |
| 2676 | UCAUGGU | | TAATTTCATGGTATAGAAAATA | | |
| ACE2_ | CUUAAUAAGGAUU | 1945 | ACTTCTGTTCTGTTTCTTAATAAG | 2145 | human |
| 2856 | UUGUAUU | | GATTTTGTATTAGAGTATATT | | |
| ACE2_ | CCCAAGUUCAAAG | 1946 | TTGAATAGCGCCCAACCCAAGTT | 2146 | human |
| 122 | GCUGAUA | | CAAAGGCTGATAAGAGAGAAAA | | |
| ACE2_ | UCUGCCAUUUACU | 1947 | CACGATTGTTGGGACTCTGCCAT | 2147 | human |
| 1564 | UACAUGU | | TTACTTACATGTTAGAGAAGTG | | |
| ACE2_ | AAGGCCACUGCUC | 1948 | AAAGAACATGAATGTAAGGCCA | 2148 | human |
| 1960 | AACUACU | | CTGCTCAACTACTTTGAGCCCTT | | |
| ACE2_ | GGGACGAUGUCAA | 1949 | GATCTTGGCTCACAGGGGACGAT | 2149 | human |
| 212 | GCUCUUC | | GTCAAGCTCTTCCTGGCTCCTT | | |
| ACE2_ | CACGAAGCCGAAG | 1950 | TTGGACAAGTTTAACCACGAAGC | 2150 | human |
| 317 | ACCUGUU | | CGAAGACCTGTTCTATCAAAGT | | |
| ACE2_ | ACAAGAAAUUCAG | 1951 | CCAAATGTATCCACTACAAGAA | 2151 | human |
| 472 | AAUCUCA | | ATTCAGAATCTCACAGTCAAGCT | | |
| ACE2_ | UGUGUCUGAUAUC | 1952 | CACTGCACCTAAAAATGTGTCTG | 2152 | human |
| 2287 | AUUCCUA | | ATATCATTCCTAGAACTGAAGT | | |
| ACE2_ | CAGGGAUAAUCUA | 1953 | GTCTCACAGGCTGTTCAGGGATA | 2153 | human |
| 2919 | AAUGUAA | | ATCTAAATGTAAATGTCTGTTG | | |
| ACE2_ | CUGAUAGAAACUC | 1954 | ACTCCCAGAGCATGCCTGATAGA | 2154 | human |
| 3334 | AUUUCUA | | AACTCATTTCTACTGTTCTCTA | | |
| ACE2_ | UAUGAUAUGGCAU | 1955 | ATGGGGCATATCCAGTATGATAT | 2155 | human |
| 1358 | AUGCUGC | | GGCATATGCTGCACAACCTTTT | | |
| ACE2_ | CACGAUUGUUGGG | 1956 | GCTCAAACAAGCACTCACGATTG | 2156 | human |
| 1549 | ACUCUGC | | TTGGGACTCTGCCATTTACTTA | | |
| ACE2_ | GAUGUUCAGACCU | 1957 | TTCCAAAACACTGATGATGTTCA | 2157 | human |
| 2615 | CCUUUUA | | GACCTCCTTTTAGAAAAATCTA | | |
| ACE2_ | AUAAGAUGAUAAA | 1958 | CATGGTATAGAAAATATAAGAT | 2158 | human |
| 2705 | GAUAUCA | | GATAAAGATATCATTAAATGTCA | | |
| ACE2_ | UGAUAAAGAUAUC | 1959 | ATAGAAAATATAAGATGATAAA | 2159 | human |
| 2711 | AUUAAAU | | GATATCATTAAATGTCAAAACTA | | |
| ACE2_ | GUUUCUUAAUAAG | 1960 | TTTGACTTCTGTTCTGTTTCTTAA | 2160 | human |
| 2852 | GAUUUUG | | TAAGGATTTTGTATTAGAGTA | | |
| ACE2_ | UUUGUAUUAGAGU | 1961 | TTTCTTAATAAGGATTTTGTATT | 2161 | human |
| 2868 | AUAUUAG | | AGAGTATATTAGGGAAAGTGTG | | |
| ACE2_ | CUUGGAAUUAUAA | 1962 | AAAGTTCACTTGCTTCTTGGAAT | 2162 | human |
| 357 | CACCAAU | | TATAACACCAATATTACTGAAG | | |
| ACE2_ | AGCCGUAUCAAUG | 1963 | ATCAGGATGTCCCGGAGCCGTAT | 2163 | human |
| 2342 | AUGCUUU | | CAATGATGCTTTCCGTCTGAAT | | |
| ACE2_ | AUUUCUGUCUCUG | 1964 | TTGCTTTCAGTATTTATTTCTGTC | 2164 | human |
| 2822 | GAUUUGA | | TCTGGATTTGACTTCTGTTCT | | |
| ACE2_ | GAGCACAAAGCAG | 1965 | TGTTTGAGCAGTGCTGAGCACAA | 2165 | human |
| 3465 | ACACUCA | | AGCAGACACTCAATAAATGCTA | | |
| ACE2_ | AAAGAUAUCAUUA | 1966 | AAAATATAAGATGATAAAGATA | 2166 | human |
| 2715 | AAUGUCA | | TCATTAAATGTCAAAACTATGAC | | |
| ACE2_ | GUGACCUUGACUG | 1967 | CTTTCTTACTTCCACGTGACCTTG | 2167 | human |
| 90 | AGUUUUG | | ACTGAGTTTTGAATAGCGCCC | | |
| ACE2_ | UGUGGAACAUACC | 1968 | CCAGTTGATTGAAGATGTGGAAC | 2168 | human |
| 892 | UUUGAAG | | ATACCTTTGAAGAGATTAAACC | | |
| ACE2_ | UAUGCUAUGAGGC | 1969 | CGATCATCTGTTGCATATGCTAT | 2169 | human |
| 2162 | AGUACUU | | GAGGCAGTACTTTTTAAAAGTA | | |
| ACE2_ | UUCCAUAUGGCUG | 1970 | TAACCAGCCCCCTGTTTCCATAT | 2170 | human |
| 2434 | AUUGUUU | | GGCTGATTGTTTTTGGAGTTGT | | |
| ACE2_ | ACUACAAUGAGAG | 1971 | TGGCAAACAGTTTAGACTACAAT | 2171 | human |
| 687 | GCUCUGG | | GAGAGGCTCTGGGCTTGGGAAA | | |
| ACE2_ | AUUUGAAACCAAG | 1972 | ATGTGCGAGTGGCTAATTTGAAA | 2172 | human |
| 2238 | AAUCUCC | | CCAAGAATCTCCTTTAATTTCT | | |
| ACE2_ | UGUUGAAUUUCUG | 1973 | CTAAATGTAAATGTCTGTTGAAT | 2173 | human |
| 2944 | AAGUUGA | | TTCTGAAGTTGAAAACAAGGAT | | |
| ACE2_ | CCCAGUCUCUUAA | 1974 | CCTCTGAAGTGGGTACCCAGTCT | 2174 | human |
| 3416 | AUCUUUU | | CTTAAATCTTTTGTATTTGCTC | | |
| ACE2_ | GAUGGGAGUGAUA | 1975 | TGTTTTTGGAGTTGTGATGGGAG | 2175 | human |
| 2464 | GUGGUUG | | TGATAGTGGTTGGCATTGTCAT | | |
| ACE2_ | UGAUGAUGUUCAG | 1976 | AGGATTCCAAAACACTGATGAT | 2176 | human |
| 2611 | ACCUCCU | | GTTCAGACCTCCTTTTAGAAAAA | | |
| ACE2_ | UGCUUCUUGGAAU | 1977 | CTATCAAAGTTCACTTGCTTCTT | 2177 | human |
| 352 | UAUAACA | | GGAATTATAACACCAATATTAC | | |
| ACE2_ | CUAAAUGUAAAUG | 1978 | CTGTTCAGGGATAATCTAAATGT | 2178 | human |
| 2929 | UCUGUUG | | AAATGTCTGTTGAATTTCTGAA | | |
| ACE2_ | AAUGCUGGGGACA | 1979 | GTCCAAAACATGAATAATGCTG | 2179 | human |
| 407 | AAUGGUC | | GGGACAAATGGTCTGCCTTTTTA | | |
| ACE2_ | UUUUCAAGAAGAC | 1980 | TCTTCTGTCACCCGATTTTCAAG | 2180 | human |
| 1498 | AAUGAAA | | AAGACAATGAAACAGAAATAAA | | |
| ACE2_ | UCUUGGAGAUAAA | 1981 | AAGCCTAAAATCAGCTCTTGGAG | 2181 | human |
| 2098 | GCAUAUG | | ATAAAGCATATGAATGGAACGA | | |
| ACE2_ | AAUGAAAUGUACC | 1982 | TATGAATGGAACGACAATGAAA | 2182 | human |
| 2129 | UGUUCCG | | TGTACCTGTTCCGATCATCTGTT | | |
| ACE2_ | AAUAUAAGAUGAU | 1983 | TTTCATGGTATAGAAAATATAAG | 2183 | human |
| 2702 | AAAGAUA | | ATGATAAAGATATCATTAAATG | | |
| ACE2_ | UGUUCUGUUUCUU | 1984 | TCTGGATTTGACTTCTGTTCTGTT | 2184 | human |
| 2846 | AAUAAGG | | TCTTAATAAGGATTTTGTATT | | |
| ACE2_ | UUUGCCUACAGUG | 1985 | CAAGTACTATGGTGATTTGCCTA | 2185 | human |
| 3156 | AUGUUUG | | CAGTGATGTTTGGAATCGATCA | | |
| ACE2_ | AAUCUCAUGAGGA | 1986 | GCTGATAAGAGAGAAAATCTCA | 2186 | human |
| 150 | GGUUUUA | | TGAGGAGGTTTTAGTCTAGGGAA | | |
| ACE2_ | UAAGAUGAUAAAG | 1987 | ATGGTATAGAAAATATAAGATG | 2187 | human |
| 2706 | AUAUCAU | | ATAAAGATATCATTAAATGTCAA | | |
| ACE2_ | GAUGUUUGGAAUC | 1988 | TGATTTGCCTACAGTGATGTTTG | 2188 | human |
| 3168 | GAUCAUG | | GAATCGATCATGCTTTCTTCAA | | |
| ACE2_ | CCACACUUGCCCA | 1989 | TTTTAAAGGAACAGTCCACACTT | 2189 | human |
| 447 | AAUGUAU | | GCCCAAATGTATCCACTACAAG | | |
| ACE2_ | GAGACUAUGAAGU | 1990 | GGGATTATTGGAGAGGAGACTA | 2190 | human |
| 831 | AAAUGGG | | TGAAGTAAATGGGGTAGATGGCT | | |
| ACE2_ | CUUCAUUGACAUU | 1991 | CCAAGGAGAGAGCATCTTCATTG | 2191 | human |
| 2796 | GCUUUCA | | ACATTGCTTTCAGTATTTATTT | | |
| ACE2_ | AAAUGUCUGUUGA | 1992 | GGATAATCTAAATGTAAATGTCT | 2192 | human |
| 2937 | AUUUCUG | | GTTGAATTTCTGAAGTTGAAAA | | |
| ACE2_ | UGUAGCUGCAAGG | 1993 | GTGCCTGGGAACTGGTGTAGCTG | 2193 | human |
| 3049 | AUUGAGA | | CAAGGATTGAGAATGGCATGCA | | |
| ACE2_ | GUUGACAUAGAUA | 1994 | TACAGAGGATCAGGAGTTGACA | 2194 | human |
| 35 | CUCUUUG | | TAGATACTCTTTGGATTTCATAC | | |
| ACE2_ | UGUCCAAAACAUG | 1995 | TATTACTGAAGAGAATGTCCAAA | 2195 | human |
| 391 | AAUAAUG | | ACATGAATAATGCTGGGGACAA | | |
| ACE2_ | GAUGAUAAAGAUA | 1996 | GTATAGAAAATATAAGATGATA | 2196 | human |
| 2709 | UCAUUAA | | AAGATATCATTAAATGTCAAAAC | | |
| ACE2_ | UUGUAUUAGAGUA | 1997 | TTCTTAATAAGGATTTTGTATTA | 2197 | human |
| 2869 | UAUUAGG | | GAGTATATTAGGGAAAGTGTGT | | |
| ACE2_ | UUCACAGUAACUC | 1998 | GGATGACATGCTTTCTTCACAGT | 2198 | human |
| 3124 | AGUUCAA | | AACTCAGTTCAAGTACTATGGT | | |
| ACE2_ | UCAGGAGUUGACA | 1999 | CATGGCTACAGAGGATCAGGAG | 2199 | human |
| 29 | UAGAUAC | | TTGACATAGATACTCTTTGGATT | | |
| ACE2_ | UGUACUCUUUGAC | 2000 | GATTTTGGACAAATCTGTACTCT | 2200 | human |
| 1050 | AGUUCCC | | TTGACAGTTCCCTTTGGACAGA | | |
| ACE2_ | GGAGCUAAUGAAG | 2001 | TTTCTGCTAAGAAATGGAGCTAA | 2201 | human |
| 1400 | GAUUCCA | | TGAAGGATTCCATGAAGCTGTT | | |
| ACE2_ | UGAGCCCUUAUUU | 2002 | ACTGCTCAACTACTTTGAGCCCT | 2202 | human |
| 1981 | ACCUGGC | | TATTTACCTGGCTGAAAGACCA | | |
| ACE2_ | UGCUAUGAGGCAG | 2003 | ATCATCTGTTGCATATGCTATGA | 2203 | human |
| 2164 | UACUUUU | | GGCAGTACTTTTTAAAAGTAAA | | |
| ACE2_ | GUAUGUAAAUGUU | 2004 | TGAGGTGATTTTGTTGTATGTAA | 2204 | human |
| 2672 | AAUUUCA | | ATGTTAATTTCATGGTATAGAA | | |
| ACE2_ | UCUUCAUUGACAU | 2005 | GCCAAGGAGAGAGCATCTTCATT | 2205 | human |
| 2795 | UGCUUUC | | GACATTGCTTTCAGTATTTATT | | |
| ACE2_ | UUUGGUCUCACAG | 2006 | TAGGGAAAGTGTGTATTTGGTCT | 2206 | human |
| 2900 | GCUGUUC | | CACAGGCTGTTCAGGGATAATC | | |
| ACE2_ | UAAGAGAGAAAAU | 2007 | AAGTTCAAAGGCTGATAAGAGA | 2207 | human |
| 140 | CUCAUGA | | GAAAATCTCATGAGGAGGTTTTA | | |
| ACE2_ | CAUGAGAUGGGGC | 2008 | TTCCTGACAGCTCATCATGAGAT | 2208 | human |
| 1337 | AUAUCCA | | GGGGCATATCCAGTATGATATG | | |
| ACE2_ | UUCUAAUGAUUAC | 2009 | ATCTCTGTTCCATGTTTCTAATG | 2209 | human |
| 1735 | UCAUUCA | | ATTACTCATTCATTCGATATTA | | |
| ACE2_ | GUAUCAAUGAUGC | 2010 | GGATGTCCCGGAGCCGTATCAAT | 2210 | human |
| 2346 | UUUCCGU | | GATGCTTTCCGTCTGAATGACA | | |
| ACE2_ | GUUGAAAACAAGG | 2011 | TGTTGAATTTCTGAAGTTGAAAA | 2211 | human |
| 2959 | AUAUAUC | | CAAGGATATATCATTGGAGCAA | | |
| ACE2_ | GAAGAUGUGGAAC | 2012 | CGCGGCCAGTTGATTGAAGATGT | 2212 | human |
| 887 | AUACCUU | | GGAACATACCTTTGAAGAGATT | | |
| ACE2_ | UUUGAAGAGAUUA | 2013 | GATGTGGAACATACCTTTGAAGA | 2213 | human |
| 905 | AACCAUU | | GATTAAACCATTATATGAACAT | | |
| ACE2_ | AGAACAAGAAUUC | 2014 | CCTGGCTGAAAGACCAGAACAA | 2214 | human |
| 2010 | UUUUGUG | | GAATTCTTTTGTGGGATGGAGTA | | |
| ACE2_ | UAGCAAAGGAGAA | 2015 | TGCCTCCATCGATATTAGCAAAG | 2215 | human |
| 2575 | AAUAAUC | | GAGAAAATAATCCAGGATTCCA | | |
| ACE2_ | GAAAACAAGGAUA | 2016 | TGAATTTCTGAAGTTGAAAACAA | 2216 | human |
| 2962 | UAUCAUU | | GGATATATCATTGGAGCAAGTG | | |
| ACE2_ | CAUGCUUUCUUCA | 2017 | ATGTTTGGAATCGATCATGCTTT | 2217 | human |
| 3184 | AGGUGAC | | CTTCAAGGTGACAGGTCTAAAG | | |
| ACE2_ | AACCCAAGUUCAA | 2018 | TTTTGAATAGCGCCCAACCCAAG | 2218 | human |
| 120 | AGGCUGA | | TTCAAAGGCTGATAAGAGAGAA | | |
| ACE2_ | AUGAACAUCUUCA | 2019 | AGATTAAACCATTATATGAACAT | 2219 | human |
| 927 | UGCCUAU | | CTTCATGCCTATGTGAGGGCAA | | |
| ACE2_ | ACUGGGAUCAGAG | 2020 | GTCATCCTGATCTTCACTGGGAT | 2220 | human |
| 2504 | AUCGGAA | | CAGAGATCGGAAGAAGAAAAAT | | |
| ACE2_ | UCUUCAAGGUGAC | 2021 | GAATCGATCATGCTTTCTTCAAG | 2221 | human |
| 3191 | AGGUCUA | | GTGACAGGTCTAAAGAGAGAAG | | |
| ACE2_ | UCAGCAAAAUGGG | 2022 | TCAGCTGCAGGCTCTTCAGCAAA | 2222 | human |
| 517 | UCUUCAG | | ATGGGTCTTCAGTGCTCTCAGA | | |
| ACE2_ | GCAAACAGUUUAG | 2023 | TTGAATGAAATAATGGCAAACA | 2223 | human |
| 674 | ACUACAA | | GTTTAGACTACAATGAGAGGCTC | | |
| ACE2_ | AAACCAUUAUAUG | 2024 | ACCTTTGAAGAGATTAAACCATT | 2224 | human |
| 917 | AACAUCU | | ATATGAACATCTTCATGCCTAT | | |
| ACE2_ | UCUGUACUCUUUG | 2025 | TAGATTTTGGACAAATCTGTACT | 2225 | human |
| 1048 | ACAGUUC | | CTTTGACAGTTCCCTTTGGACA | | |
| ACE2_ | UUUGACAGUUCCC | 2026 | GACAAATCTGTACTCTTTGACAG | 2226 | human |
| 1057 | UUUGGAC | | TTCCCTTTGGACAGAAACCAAA | | |
| ACE2_ | GAAACAGAAAUAA | 2027 | TTTCAAGAAGACAATGAAACAG | 2227 | human |
| 1514 | ACUUCCU | | AAATAAACTTCCTGCTCAAACAA | | |
| ACE2_ | CAGUAUUUAUUUC | 2028 | CATTGACATTGCTTTCAGTATTT | 2228 | human |
| 2814 | UGUCUCU | | ATTTCTGTCTCTGGATTTGACT | | |
| ACE2_ | CAGUUCAAGUACU | 2029 | TTCTTCACAGTAACTCAGTTCAA | 2229 | human |
| 3136 | AUGGUGA | | GTACTATGGTGATTTGCCTACA | | |
| ACE2_ | GUCCAAAACAUGA | 2030 | ATTACTGAAGAGAATGTCCAAA | 2230 | human |
| 392 | AUAAUGC | | ACATGAATAATGCTGGGGACAAA | | |
| ACE2_ | UUGAUGAAUGCCU | 2031 | TATGTGAGGGCAAAGTTGATGA | 2231 | human |
| 959 | AUCCUUC | | ATGCCTATCCTTCCTATATCAGT | | |
| ACE2_ | UGCAUAUGCUAUG | 2032 | GTTCCGATCATCTGTTGCATATG | 2232 | human |
| 2158 | AGGCAGU | | CTATGAGGCAGTACTTTTTAAA | | |
| ACE2_ | UAUGCCUCCAUCG | 2033 | AGTGGAGAAAATCCTTATGCCTC | 2233 | human |
| 2558 | AUAUUAG | | CATCGATATTAGCAAAGGAGAA | | |
| ACE2_ | AGUUGACAUAGAU | 2034 | CTACAGAGGATCAGGAGTTGAC | 2234 | human |
| 34 | ACUCUUU | | ATAGATACTCTTTGGATTTCATA | | |
| ACE2_ | UUUAGUCUAGGGA | 2035 | ATCTCATGAGGAGGTTTTAGTCT | 2235 | human |
| 166 | AAGUCAU | | AGGGAAAGTCATTCAGTGGATG | | |
| ACE2_ | UCCACACUUGCCC | 2036 | TTTTTAAAGGAACAGTCCACACT | 2236 | human |
| 446 | AAAUGUA | | TGCCCAAATGTATCCACTACAA | | |
| ACE2_ | UCUUCUGUCACCC | 2037 | TTTAAAATCCATTGGTCTTCTGT | 2237 | human |
| 1483 | GAUUUUC | | CACCCGATTTTCAAGAAGACAA | | |
| ACE2_ | AUAGAAAAUAUAA | 2038 | TGTTAATTTCATGGTATAGAAAA | 2238 | human |
| 2696 | GAUGAUA | | TATAAGATGATAAAGATATCAT | | |
| ACE2_ | UAUGCUGCACAAC | 2039 | CAGTATGATATGGCATATGCTGC | 2239 | human |
| 1370 | CUUUUCU | | ACAACCTTTTCTGCTAAGAAAT | | |
| ACE2_ | UGGAUUUGACUUC | 2040 | ATTTATTTCTGTCTCTGGATTTGA | 2240 | human |
| 2833 | UGUUCUG | | CTTCTGTTCTGTTTCTTAATA | | |
| ACE2_ | GACUUCUGUUCUG | 2041 | TCTGTCTCTGGATTTGACTTCTGT | 2241 | human |
| 2840 | UUUCUUA | | TCTGTTTCTTAATAAGGATTT | | |
| ACE2_ | CUUUCAUUUAAUC | 2042 | GCATGCATTAGCTCACTTTCATT | 2242 | human |
| 3087 | CAUUGUC | | TAATCCATTGTCAAGGATGACA | | |
| ACE2_ | AAGGAUGACAUGC | 2043 | ATTTAATCCATTGTCAAGGATGA | 2243 | human |
| 3107 | UUUCUUC | | CATGCTTTCTTCACAGTAACTC | | |
| ACE2_ | AGGUAGAGGACAU | 2044 | AAGAATCCAGGGAACAGGTAGA | 2244 | human |
| 3233 | UGCUUUU | | GGACATTGCTTTTTCACTTCCAA | | |
| ACE2_ | UUUGUAUCUGUUG | 2045 | GAGGCCGAGAAGTTCTTTGTATC | 2245 | human |
| 1160 | GUCUUCC | | TGTTGGTCTTCCTAATATGACT | | |
| ACE2_ | AUUCCCAAAGACC | 2046 | GTCTTTAAAGGGGAAATTCCCAA | 2246 | human |
| 1619 | AGUGGAU | | AGACCAGTGGATGAAAAAGTGG | | |
| ACE2_ | AAGACCAGAACAA | 2047 | TATTTACCTGGCTGAAAGACCAG | 2247 | human |
| 2004 | GAAUUCU | | AACAAGAATTCTTTTGTGGGAT | | |
| ACE2_ | UAAAGCAUAUGAA | 2048 | ATCAGCTCTTGGAGATAAAGCAT | 2248 | human |
| 2107 | UGGAACG | | ATGAATGGAACGACAATGAAAT | | |
| ACE2_ | CUGGAUUUGACUU | 2049 | TATTTATTTCTGTCTCTGGATTTG | 2249 | human |
| 2832 | CUGUUCU | | ACTTCTGTTCTGTTTCTTAAT | | |
| ACE2_ | UAGGGAAAGUGUG | 2050 | TGTATTAGAGTATATTAGGGAAA | 2250 | human |
| 2885 | UAUUUGG | | GTGTGTATTTGGTCTCACAGGC | | |
| ACE2_ | GCAUGCAUUAGCU | 2051 | CAAGGATTGAGAATGGCATGCA | 2251 | human |
| 3072 | CACUUUC | | TTAGCTCACTTTCATTTAATCCA | | |
| ACE2_ | UUGGUCUUCCUAA | 2052 | AGTTCTTTGTATCTGTTGGTCTTC | 2252 | human |
| 1170 | UAUGACU | | CTAATATGACTCAAGGATTCT | | |
| ACE2_ | GUUUCUAAUGAUU | 2053 | GCATCTCTGTTCCATGTTTCTAAT | 2253 | human |
| 1733 | ACUCAUU | | GATTACTCATTCATTCGATAT | | |
| ACE2_ | UUACACAAGGACC | 2054 | CTCATTCATTCGATATTACACAA | 2254 | human |
| 1762 | CUUUACC | | GGACCCTTTACCAATTCCAGTT | | |
| ACE2_ | UUGAAACCAAGAA | 2055 | GTGCGAGTGGCTAATTTGAAACC | 2255 | human |
| 2240 | UCUCCUU | | AAGAATCTCCTTTAATTTCTTT | | |
| ACE2_ | UCUGAUAUCAUUC | 2056 | GCACCTAAAAATGTGTCTGATAT | 2256 | human |
| 2291 | CUAGAAC | | CATTCCTAGAACTGAAGTTGAA | | |
| ACE2_ | AGUGCCUGGGAAC | 2057 | ATCACTTGTAAGGACAGTGCCTG | 2257 | human |
| 3033 | UGGUGUA | | GGAACTGGTGTAGCTGCAAGGA | | |
| ACE2_ | UGAGAAUGGCAUG | 2058 | TGTAGCTGCAAGGATTGAGAAT | 2258 | human |
| 3064 | CAUUAGC | | GGCATGCATTAGCTCACTTTCAT | | |
| ACE2_ | UGCAUUAGCUCAC | 2059 | GGATTGAGAATGGCATGCATTA | 2259 | human |
| 3075 | UUUCAUU | | GCTCACTTTCATTTAATCCATTG | | |
| ACE2_ | UCACCCUCUGAAG | 2060 | ATTCCAACTGTATGTTCACCCTC | 2260 | human |
| 3397 | UGGGUAC | | TGAAGTGGGTACCCAGTCTCTT | | |
| ACE2_ | UGAGGAGGUUUUA | 2061 | AGAGAGAAAATCTCATGAGGAG | 2261 | human |
| 157 | GUCUAGG | | GTTTTAGTCTAGGGAAAGTCATT | | |
| ACE2_ | UUUGCUUGGUGAU | 2062 | ATGCCTCCCTGCTCATTTGCTTG | 2262 | human |
| 1012 | AUGUGGG | | GTGATATGTGGGGTAGATTTTG | | |
| ACE2_ | UGGGGCAUAUCCA | 2063 | CAGCTCATCATGAGATGGGGCAT | 2263 | human |
| 1344 | GUAUGAU | | ATCCAGTATGATATGGCATATG | | |
| ACE2_ | AUAUGAAUGGAAC | 2064 | TCTTGGAGATAAAGCATATGAAT | 2264 | human |
| 2113 | GACAAUG | | GGAACGACAATGAAATGTACCT | | |
| ACE2_ | UUUCAUGGUAUAG | 2065 | GTATGTAAATGTTAATTTCATGG | 2265 | human |
| 2687 | AAAAUAU | | TATAGAAAATATAAGATGATAA | | |
| ACE2_ | UACUGAAGAGAAU | 2066 | TTATAACACCAATATTACTGAAG | 2266 | human |
| 379 | GUCCAAA | | AGAATGTCCAAAACATGAATAA | | |
| ACE2_ | GGCAAGAGCAAAU | 2067 | CTTGAAAAATGAGATGGCAAGA | 2267 | human |
| 787 | CAUUAUG | | GCAAATCATTATGAGGACTATGG | | |
| ACE2_ | UGCUGCACAACCU | 2068 | GTATGATATGGCATATGCTGCAC | 2268 | human |
| 1372 | UUUCUGC | | AACCTTTTCTGCTAAGAAATGG | | |
| ACE2_ | AAAGACCAGAACA | 2069 | TTATTTACCTGGCTGAAAGACCA | 2269 | human |
| 2003 | AGAAUUC | | GAACAAGAATTCTTTTGTGGGA | | |
| ACE2_ | AACGACAAUGAAA | 2070 | AAAGCATATGAATGGAACGACA | 2270 | human |
| 2123 | UGUACCU | | ATGAAATGTACCTGTTCCGATCA | | |
| ACE2_ | UGUGCGAGUGGCU | 2071 | TTTTGGGGAGGAGGATGTGCGA | 2271 | human |
| 2224 | AAUUUGA | | GTGGCTAATTTGAAACCAAGAAT | | |
| ACE2_ | UUAGCAAAGGAGA | 2072 | ATGCCTCCATCGATATTAGCAAA | 2272 | human |
| 2574 | AAAUAAU | | GGAGAAAATAATCCAGGATTCC | | |
| ACE2_ | GGAACAGGUAGAG | 2073 | GAGAGAAGAATCCAGGGAACAG | 2273 | human |
| 3228 | GACAUUG | | GTAGAGGACATTGCTTTTTCACT | | |
| ACE2_ | CUUAAAUCUUUUG | 2074 | GTGGGTACCCAGTCTCTTAAATC | 2274 | human |
| 3424 | UAUUUGC | | TTTTGTATTTGCTCACAGTGTT | | |
| ACE2_ | UAUCCACUACAAG | 2075 | ACACTTGCCCAAATGTATCCACT | 2275 | human |
| 464 | AAAUUCA | | ACAAGAAATTCAGAATCTCACA | | |
| ACE2_ | AAACCAAACAUAG | 2076 | GTTCCCTTTGGACAGAAACCAAA | 2276 | human |
| 1079 | AUGUUAC | | CATAGATGTTACTGATGCAATG | | |
| ACE2_ | CAAGGAUUCUGGG | 2077 | CTTCCTAATATGACTCAAGGATT | 2277 | human |
| 1190 | AAAAUUC | | CTGGGAAAATTCCATGCTAACG | | |
| ACE2_ | ACCAGAACAAGAA | 2078 | TTACCTGGCTGAAAGACCAGAA | 2278 | human |
| 2007 | UUCUUUU | | CAAGAATTCTTTTGTGGGATGGA | | |
| ACE2_ | AUGUUAAUUUCAU | 2079 | TTTTGTTGTATGTAAATGTTAATT | 2279 | human |
| 2680 | GGUAUAG | | TCATGGTATAGAAAATATAAG | | |
| ACE2_ | GAUAUCAUUAAAU | 2080 | ATATAAGATGATAAAGATATCAT | 2280 | human |
| 2718 | GUCAAAA | | TAAATGTCAAAACTATGACTCT | | |
| ACE2_ | UUAAAUGUCAAAA | 2081 | ATGATAAAGATATCATTAAATGT | 2281 | human |
| 2725 | CUAUGAC | | CAAAACTATGACTCTGTTCAGA | | |
| ACE2_ | CAGGCUGUUCAGG | 2082 | GTGTATTTGGTCTCACAGGCTGT | 2282 | human |
| 2910 | GAUAAUC | | TCAGGGATAATCTAAATGTAAA | | |
| ACE2_ | UGUCAAGGAUGAC | 2083 | TTTCATTTAATCCATTGTCAAGG | 2283 | human |
| 3103 | AUGCUUU | | ATGACATGCTTTCTTCACAGTA | | |
| ACE2_ | AUGGUGAUUUGCC | 2084 | CTCAGTTCAAGTACTATGGTGAT | 2284 | human |
| 3149 | UACAGUG | | TTGCCTACAGTGATGTTTGGAA | | |
| ACE2_ | UUGGACAAAUCUG | 2085 | TATGTGGGGTAGATTTTGGACAA | 2285 | human |
| 1039 | UACUCUU | | ATCTGTACTCTTTGACAGTTCC | | |
| ACE2_ | UCAAGAAGCACUU | 2086 | TTACCAATTCCAGTTTCAAGAAG | 2286 | human |
| 1792 | UGUCAAG | | CACTTTGTCAAGCAGCTAAACA | | |
| ACE2_ | UUUAAUUUCUUUG | 2087 | AAACCAAGAATCTCCTTTAATTT | 2287 | human |
| 2258 | UCACUGC | | CTTTGTCACTGCACCTAAAAAT | | |
| ACE2_ | GCAAGGAUUGAGA | 2088 | GGAACTGGTGTAGCTGCAAGGA | 2288 | human |
| 3056 | AUGGCAU | | TTGAGAATGGCATGCATTAGCTC | | |
| ACE2_ | UUGGAAUCGAUCA | 2089 | TGCCTACAGTGATGTTTGGAATC | 2289 | human |
| 3173 | UGCUUUC | | GATCATGCTTTCTTCAAGGTGA | | |
| ACE2_ | UCUCAUGAGGAGG | 2090 | TGATAAGAGAGAAAATCTCATG | 2290 | human |
| 152 | UUUUAGU | | AGGAGGTTTTAGTCTAGGGAAAG | | |
| ACE2_ | CAUUAUAUGAAGA | 2091 | GCAAGCAGCTGAGGCCATTATAT | 2291 | human |
| 750 | GUAUGUG | | GAAGAGTATGTGGTCTTGAAAA | | |
| ACE2_ | UCUGGGAAAAUUC | 2092 | ATATGACTCAAGGATTCTGGGAA | 2292 | human |
| 1197 | CAUGCUA | | AATTCCATGCTAACGGACCCAG | | |
| ACE2_ | UUCAGGAUCCUUA | 2093 | CTGGGGAAGGGCGACTTCAGGA | 2293 | human |
| 1283 | UGUGCAC | | TCCTTATGTGCACAAAGGTGACA | | |
| ACE2_ | AGGCCCUCUGCAC | 2094 | AGCAGCTAAACATGAAGGCCCT | 2294 | human |
| 1825 | AAAUGUG | | CTGCACAAATGTGACATCTCAAA | | |

TABLE 12A
Host targets screened-TMPRSS-20 nucleotide targets and 45 nucleotide gene
target regions

| | | SEQ | | SEQ | |
|---|---|---|---|---|---|
| Sequence | | ID | | ID | |
| ID | Sequence | NO: | Gene Region | NO: | Species |
| TMPRSS | UGUGAAAAUGA | 2295 | ACTGTAAAGTTCAATTGTGAAAAT | 2331 | human |
| 2_3153 | AUAUCAUGC | | GAATATCATGCAAATAAATTA | | |
| TMPRSS | ACCUUCAUUUA | 2296 | GTCTCCAAGTAGTCCACCTTCATT | 2332 | human |
| 2_2577 | ACUCUUUGA | | TAACTCTTTGAAACTGTATCA | | |
| TMPRSS | UCGUCCUUGACG | 2297 | TAATCCACATGGTCTTCGTCCTTG | 2333 | human |
| 2_1626 | UCGUUUUA | | ACGTCGTTTTACAAGAAAACA | | |
| TMPRSS | GGAGCCGGAUA | 2298 | TCTTTCATGTTCTATGGAGCCGGA | 2334 | human |
| 2_1101 | CCAAGUAGA | | TACCAAGTAGAAAAAGTGATT | | |
| TMPRSS | UACCACAGUGA | 2299 | ATCTATAAAAAACTGTACCACAGT | 2335 | human |
| 2_810 | UGCCUGUUC | | GATGCCTGTTCTTCAAAAGCA | | |
| TMPRSS | UCAUGCAAAUA | 2300 | TTGTGAAAATGAATATCATGCAAA | 2336 | human |
| 2_3167 | AAUUAUGCA | | TAAATTATGCAATTTTTTTTT | | |
| TMPRSS | UUGAAACUGUA | 2301 | CCTTCATTTAACTCTTTGAAACTGT | 2337 | human |
| 2_593 | UCAUCUUUG | | ATCATCTTTGCCAAGTAAGA | | |
| TMPRSS | GCCGGCAAUGUC | 2302 | AAACTGAACACAAGTGCCGGCAA | 2338 | human |
| 2_780 | GAUAUCUA | | TGTCGATATCTATAAAAAACTG | | |
| TMPRSS | UGUAAUGGUGA | 2303 | CATCCTAAAAGGTGTTGTAATGGT | 2339 | human |
| 2_3054 | AAACGUCUU | | GAAAACGTCTTCCTTCTTTAT | | |
| TMPRSS | GGUGGCCUAUU | 2304 | TTGCCAAGTAAGAGTGGTGGCCTA | 2340 | human |
| 2_2625 | UCAGCUGCU | | TTTCAGCTGCTTTGACAAAAT | | |
| TMPRSS | ACAGCUAGGAC | 2305 | GAAATGAATGATTCTACAGCTAGG | 2341 | human |
| 2_2899 | UUAACCUUG | | ACTTAACCTTGAAATGGAAAG | | |
| TMPRSS | CAGUUUAAGGU | 2306 | GAAATCAAGGATGCTCAGTTTAAG | 2342 | human |
| 2_2485 | ACACUGUUU | | GTACACTGTTTCCATGTTATG | | |
| TMPRSS | GCCGCCAGAGCA | 2307 | TCAACTTGAACTCAAGCCGCCAGA | 2343 | human |
| 2_886 | GGAUUGUG | | GCAGGATTGTGGGCGGCGAGA | | |
| TMPRSS | CCAGCCAUGAUC | 2308 | GACAACCTGATCACACCAGCCATG | 2344 | human |
| 2_1398 | UGUGCCGG | | ATCTGTGCCGGCTTCCTGCAG | | |
| TMPRSS | UCCAUCAUCACC | 2309 | CACGTGTGCGGAGGCTCCATCATC | 2345 | human |
| 2_984 | CCCGAGUG | | ACCCCCGAGTGGATCGTGACA | | |
| TMPRSS | AUGAUCUGUGC | 2310 | CTGATCACACCAGCCATGATCTGT | 2346 | human |
| 2_1404 | CGGCUUCCU | | GCCGGCTTCCTGCAGGGGAAC | | |
| TMPRSS | GCUUUGAACUC | 2311 | GATAACAGCAAGATGGCTTTGAA | 2347 | human |
| 2_138 | AGGGUCACC | | CTCAGGGTCACCACCAGCTATT | | |
| TMPRSS | AGGAGAAAGGG | 2312 | GGTGGGGGGCCACCGAGGAGAAA | 2348 | human |
| 2_1297 | AAGACCUCA | | GGGAAGACCTCAGAAGTGCTGA | | |
| TMPRSS | CCUGGCAGGUCA | 2313 | TCCCGGGGGCCTGGCCCTGGCAGG | 2349 | human |
| 2_937 | GCCUGCAC | | TCAGCCTGCACGTCCAGAACG | | |
| TMPRSS | GAGGAGAAAGG | 2314 | GGGTGGGGGGCCACCGAGGAGAA | 2350 | human |
| 2_1296 | GAAGACCUC | | AGGGAAGACCTCAGAAGTGCTG | | |
| TMPRSS | CAGGUCAGCCUG | 2315 | GGGGCCTGGCCCTGGCAGGTCAG | 2351 | human |
| 2_942 | CACGUCCA | | CCTGCACGTCCAGAACGTCCAC | | |
| TMPRSS | CAUUGGACGGC | 2316 | CTTAACAATCCATGGCATTGGACG | 2352 | human |
| 2_1053 | AUUUGCGGG | | GCATTTGCGGGGATTTTGAGA | | |
| TMPRSS | GAGGCUCCAUCA | 2317 | ACGTCCACGTGTGCGGAGGCTCCA | 2353 | human |
| 2_979 | UCACCCCC | | TCATCACCCCCGAGTGGATCG | | |
| TMPRSS | CAUGAUCUGUG | 2318 | CCTGATCACACCAGCCATGATCTG | 2354 | human |
| 2_1403 | CCGGCUUCC | | TGCCGGCTTCCTGCAGGGGAA | | |
| TMPRSS | ACCAGCCAUGAU | 2319 | TGACAACCTGATCACACCAGCCAT | 2355 | human |
| 2_1397 | CUGUGCCG | | GATCTGTGCCGGCTTCCTGCA | | |
| TMPRSS | AAAGCCAUGCCA | 962 | TGGGTTTATACCAGGAAAGCCATG | 1162 | mouse |
| 2_2779 | GAAUUACC | | CCAGAATTACCAAATATGAAG | | |
| TMPRSS | UUUGUCUUCAA | 2320 | TTGTCCCAGACTTCCTTTGTCTTCA | 2356 | mouse |
| 2_1730 | CAACCUUCU | | ACAACCTTCTGCAAGAAAAC | | |
| TMPRSS | UGCACAAUGUA | 2321 | AATTTTAACTTCCTGTGCACAATG | 2357 | mouse |
| 2_1785 | CCUUUUGAG | | TACCTTTTGAGATGATTCGAA | | |
| TMPRSS | UGGGACAGCAA | 2322 | TTGCTTTGGAGGTTCTGGGACAGC | 2358 | mouse |
| 2_552 | CUGUUCUAC | | AACTGTTCTACGTCTGAGATG | | |
| TMPRSS | UUCCACUGUGA | 2323 | TTCTGAGCTGTGAGATTCCACTGT | 2359 | mouse |
| 2_3120 | AAUAUAUGA | | GAAATATATGAATAAAGTATA | | |
| TMPRSS | UCAGGCAACGU | 2324 | AAGCTGAATGTGAGCTCAGGCAA | 2360 | mouse |
| 2_873 | UGACCUCUA | | CGTTGACCTCTATAAAAAACTC | | |
| TMPRSS | GGGAACGUGAC | 2325 | AGACCTGGAGTATACGGGAACGT | 2361 | mouse |
| 2_1647 | GGUAUUUAC | | GACGGTATTTACAGATTGGATC | | |
| TMPRSS | UCUGCAAGAAA | 2326 | TGTCTTCAACAACCTTCTGCAAGA | 2362 | mouse |
| 2_1747 | ACCAAGGGC | | AAACCAAGGGCCTGAATTTTA | | |
| TMPRSS | UUUGGCUUUUA | 2327 | GAAGCTGCAGACACCTTTGGCTTT | 2363 | mouse |
| 2_1286 | AUGAUCUAG | | TAATGATCTAGTGAAGCCAGT | | |
| TMPRSS | UAAGCGAGAAC | 2328 | CCGCCTCCGGAGATTTAAGCGAGA | 2364 | mouse |
| 2_88 | UGGAGUAGG | | ACTGGAGTAGGTCGTGTACTT | | |
| TMPRSS | GUUGACAUGAC | 2329 | CTTGCTCTCCTGCATGTTGACATG | 2365 | mouse |
| 2_2243 | GGCCCUUUC | | ACGGCCCTTTCCAAGGGTGAT | | |
| TMPRSS | UGCUUCUGGGU | 2330 | TGATTTCAGTCACCTTGCTTCTGG | 2366 | mouse |
| 2_2539 | UGUGUUUCU | | GTTGTGTTTCTTCTCTTACTA | | |

TABLE 12B
Host targets screened - IL-6-20 nucleotide targets and 45 nucleotide
gene target regions

| Se- | | SEQ | | SEQ | |
|---|---|---|---|---|---|
| quence | | ID | | ID | |
| ID | Sequence | NO: | Gene Region | NO: | Species |
| IL6_ | UUGGAAAGUGUA | 2367 | GAGCCAGATCATTTCTTGGAAAG | 2389 | human |
| 933 | GGCUUACC | | TGTAGGCTTACCTCAAATAAAT | | |
| IL6_ | CUUGAAAUGUUA | 2368 | TTTTATGAAGTGTCACTTGAAAT | 2390 | mouse |
| 930 | UAUGUUAU | | GTTATATGTTATAGTTTTGAAA | | |
| IL6_ | UAUGAUUGAUAU | 2369 | AACTTTAAGTTAATTTATGATTG | 2391 | mouse |
| 896 | UUAUUAUU | | ATATTTATTATTTTTATGAAGT | | |
| IL6_ | UUAAAUAAGUAA | 2370 | TAATTTATTGATAATTTAAATAA | 2392 | mouse |
| 870 | ACUUUAAG | | GTAAACTTTAAGTTAATTTATG | | |
| IL6_ | GACACUAUUUUA | 2371 | CAATATGAATGTTGGGACACTAT | 2393 | mouse |
| 835 | AUUAUUUU | | TTTAATTATTTTTAATTTATTG | | |
| IL6_ | GGACACUAUUUU | 2372 | ACAATATGAATGTTGGGACACTA | 2394 | mouse |
| 834 | AAUUAUUU | | TTTTAATTATTTTTAATTTATT | | |
| IL6_ | UGGGACACUAUU | 2373 | TGACAATATGAATGTTGGGACAC | 2395 | mouse |
| 832 | UUAAUUAU | | TATTTTAATTATTTTTAATTTA | | |
| IL6_ | GUGGACAUUCCUC | 2374 | TAAGCATATCAGTTTGTGGACAT | 2396 | mouse |
| 741 | ACUGUGG | | TCCTCACTGTGGTCAGAAAATA | | |
| IL6_ | UGAAGAAUUUCU | 2375 | CATCTTGAAATCACTTGAAGAAT | 2397 | mouse |
| 669 | AAAAGUCA | | TTCTAAAAGTCACTTTGAGATC | | |
| IL6_ | AUGGGCACCUCAG | 2376 | TTCGGCAAATGTAGCATGGGCAC | 2398 | human |
| 635 | AUUGUUG | | CTCAGATTGTTGTTGTTAATGG | | |
| IL6_ | UCGGCAAAUGUA | 2377 | CAGCCTGAGGGCTCTTCGGCAAA | 2399 | human |
| 621 | GCAUGGGC | | TGTAGCATGGGCACCTCAGATT | | |
| IL6_ | UCGGCAAAUGUA | 2377 | CAGCCTGAGGGCTCTTCGGCAAA | 2399 | human |
| 621 | GCAUGGGC | | TGTAGCATGGGCACCTCAGATT | | |
| IL6_ | GAUAUAAUCAGG | 2378 | GATGCTACCAAACTGGATATAAT | 2400 | mouse |
| 386 | AAAUUUGC | | CAGGAAATTTGCCTATTGAAAA | | |
| IL6_ | UGAGGUAUACCU | 2379 | TGGTCTTTTGGAGTTTGAGGTAT | 2401 | human |
| 360 | AGAGUACC | | ACCTAGAGTACCTCCAGAACAG | | |
| IL6_ | GGUGAAAAUCAU | 2380 | TGAGGAGACTTGCCTGGTGAAAA | 2402 | human |
| 330 | CACUGGUC | | TCATCACTGGTCTTTTGGAGTT | | |
| IL6_ | GGUGAAAAUCAU | 2380 | TGAGGAGACTTGCCTGGTGAAAA | 2402 | human |
| 330 | CACUGGUC | | TCATCACTGGTCTTTTGGAGTT | | |
| IL6_ | CUUGCCUGGUGAA | 2381 | GATTCAATGAGGAGACTTGCCTG | 2403 | human |
| 323 | AAUCAUC | | GTGAAAATCATCACTGGTCTTT | | |
| IL6_ | CUUGCCUGGUGAA | 2381 | GATTCAATGAGGAGACTTGCCTG | 2403 | human |
| 323 | AAUCAUC | | GTGAAAATCATCACTGGTCTTT | | |
| IL6_ | UGUGCAAUGGCA | 2382 | AAATGAGAAAAGAGTTGTGCAA | 2404 | mouse |
| 284 | AUUCUGAU | | TGGCAATTCTGATTGTATGAACA | | |
| IL6_ | UGAACCUUCCAAA | 2383 | TGGCAGAAAACAACCTGAACCTT | 2405 | human |
| 263 | GAUGGCU | | CCAAAGATGGCTGAAAAAGATG | | |
| IL6_ | ACUGGCAGAAAAC | 2384 | AAGCAGCAAAGAGGCACTGGCA | 2406 | human |
| 246 | AACCUGA | | GAAAACAACCTGAACCTTCCAAA | | |
| IL6_ | ACUGGCAGAAAAC | 2384 | AAGCAGCAAAGAGGCACTGGCA | 2406 | human |
| 246 | AACCUGA | | GAAAACAACCTGAACCTTCCAAA | | |
| IL6_ | GAAAGCAGCAAA | 2385 | AAGAGTAACATGTGTGAAAGCA | 2407 | human |
| 229 | GAGGCACU | | GCAAAGAGGCACTGGCAGAAAAC | | |
| IL6_ | GAAAGCAGCAAA | 2385 | AAGAGTAACATGTGTGAAAGCA | 2407 | human |
| 229 | GAGGCACU | | GCAAAGAGGCACTGGCAGAAAAC | | |
| IL6_ | UCAGCCCUGAGAA | 2386 | ATCCTCGACGGCATCTCAGCCCT | 2408 | human |
| 187 | AGGAGAC | | GAGAAAGGAGACATGTAACAAG | | |
| IL6_ | UGCUAAUUUAAA | 2387 | TTTACCTCAATGAATTGCTAATTT | 2409 | mouse |
| 1045 | UAUGUUUU | | AAATATGTTTTTAAAGAAATC | | |
| IL6_ | CUUGGAAUGUAU | 2388 | CTAGCCAGATGGTTTCTTGGAAT | 2410 | mouse |
| 1015 | AAGUUUAC | | GTATAAGTTTACCTCAATGAAT | | |

TABLE 12C
Host targets screened - ACE2_-20 nucleotide targets and 45 nucleotide
gene target regions

| Se- | | SEQ | | SEQ | |
|---|---|---|---|---|---|
| quence | | ID | | ID | |
| ID | Sequence | NO: | Gene Region | NO: | Species |
| ACE2_ | CAACCCAAGUUC | 2411 | GTTTTGAATAGCGCCCAACCCAAG | 2435 | human |
| 119 | AAAGGCUG | | TTCAAAGGCTGATAAGAGAGA | | |
| ACE2_ | UCUAUCAAAGUU | 2412 | AAGCCGAAGACCTGTTCTATCAAA | 2436 | human |
| 336 | CACUUGCU | | GTTCACTTGCTTCTTGGAATT | | |
| ACE2_ | ACUUGCUUCUUG | 2413 | GTTCTATCAAAGTTCACTTGCTTCT | 2437 | human |
| 349 | GAAUUAUA | | TGGAATTATAACACCAATAT | | |
| ACE2_ | UUGGAAUUAUAA | 2414 | AAGTTCACTTGCTTCTTGGAATTA | 2438 | human |
| 358 | CACCAAUA | | TAACACCAATATTACTGAAGA | | |
| ACE2_ | AAUCCACAAGAA | 2415 | GTTTGTAACCCAGATAATCCACAA | 2439 | human |
| 626 | UGCUUAUU | | GAATGCTTATTACTTGAACCA | | |
| ACE2_ | GAUGGCAAGAGC | 2416 | GGTCTTGAAAAATGAGATGGCAA | 2440 | human |
| 784 | AAAUCAUU | | GAGCAAATCATTATGAGGACTA | | |
| ACE2_ | GAAGAGAUUAAA | 2417 | GTGGAACATACCTTTGAAGAGATT | 2441 | human |
| 908 | CCAUUAUA | | AAACCATTATATGAACATCTT | | |
| ACE2_ | AGAUUUUGGACA | 2418 | GGTGATATGTGGGGTAGATTTTGG | 2442 | human |
| 1034 | AAUCUGUA | | ACAAATCTGTACTCTTTGACA | | |
| ACE2_ | UUGGACAGAAAC | 2419 | CTTTGACAGTTCCCTTTGGACAGA | 2443 | human |
| 1071 | CAAACAUA | | AACCAAACATAGATGTTACTG | | |
| ACE2_ | CCUAAUAUGACU | 2420 | GTATCTGTTGGTCTTCCTAATATG | 2444 | human |
| 1178 | CAAGGAUU | | ACTCAAGGATTCTGGGAAAAT | | |
| ACE2_ | UCUGCUAAGAAA | 2421 | TGCTGCACAACCTTTTCTGCTAAG | 2445 | human |
| 1387 | UGGAGCUA | | AAATGGAGCTAATGAAGGATT | | |
| ACE2_ | GGGGAAAUCAUG | 2422 | TTCCATGAAGCTGTTGGGGAAATC | 2446 | human |
| 1430 | UCACUUUC | | ATGTCACTTTCTGCAGCCACA | | |
| ACE2_ | UGAAACAGAAAU | 2423 | TTTTCAAGAAGACAATGAAACAG | 2447 | human |
| 1513 | AAACUUCC | | AAATAAACTTCCTGCTCAAACA | | |
| ACE2_ | UCACGAUUGUUG | 2424 | TGCTCAAACAAGCACTCACGATTG | 2448 | human |
| 1548 | GGACUCUG | | TTGGGACTCTGCCATTTACTT | | |
| ACE2_ | GUGGAGGUGGAU | 2425 | TTACATGTTAGAGAAGTGGAGGTG | 2449 | human |
| 1591 | GGUCUUUA | | GATGGTCTTTAAAGGGGAAAT | | |
| ACE2_ | CUAAUGAUUACU | 2426 | CTCTGTTCCATGTTTCTAATGATTA | 2450 | human |
| 1737 | CAUUCAUU | | CTCATTCATTCGATATTACA | | |
| ACE2_ | UUCGAUAUUACA | 2427 | ATGATTACTCATTCATTCGATATT | 2451 | human |
| 1755 | CAAGGACC | | ACACAAGGACCCTTTACCAAT | | |
| ACE2_ | CUUUACCAAUUC | 2428 | TATTACACAAGGACCCTTTACCAA | 2452 | human |
| 1775 | CAGUUUCA | | TTCCAGTTTCAAGAAGCACTT | | |
| ACE2_ | CUGCACAAAUGU | 2429 | AAACATGAAGGCCCTCTGCACAA | 2453 | human |
| 1832 | GACAUCUC | | ATGTGACATCTCAAACTCTACA | | |
| ACE2_ | UAGAAAAUAUAA | 2430 | GTTAATTTCATGGTATAGAAAATA | 2454 | human |
| 2697 | GAUGAUAA | | TAAGATGATAAAGATATCATT | | |
| ACE2_ | AUGGCCAAGGAG | 2431 | TTGTCCAAAGACAACATGGCCAAG | 2455 | human |
| 2777 | AGAGCAUC | | GAGAGAGCATCTTCATTGACA | | |
| ACE2_ | AUUGACAUUGCU | 2432 | GGAGAGAGCATCTTCATTGACATT | 2456 | human |
| 2800 | UUCAGUAU | | GCTTTCAGTATTTATTTCTGT | | |
| ACE2_ | UUCAGUAUUUAU | 2433 | TTCATTGACATTGCTTTCAGTATTT | 2457 | human |
| 2812 | UUCUGUCU | | ATTTCTGTCTCTGGATTTGA | | |
| ACE2_ | UUUGGAAUCGAU | 2434 | TTGCCTACAGTGATGTTTGGAATC | 2458 | human |
| 3172 | CAUGCUUU | | GATCATGCTTTCTTCAAGGTG | | |

TABLE 12D
Host targets screened - FURIN-20 nucleotide targets and 45 nucleotide
gene target regions

| Se- | | SEQ | | SEQ | |
|---|---|---|---|---|---|
| quence | | ID | | ID | |
| ID | Sequence | NO: | Gene Region | NO: | Species |
| FURIN_ | UCAACCUGGG | 2459 | GGAAGCATGGGTTCCTCAACCTGG | 2483 | human |
| 443 | CCAGAUCUUC | | GCCAGATCTTCGGGGACTATT | | |
| FURIN_ | AUUACCACUU | 2460 | AGATCTTCGGGGACTATTACCACTT | 2484 | human |
| 470 | CUGGCAUCGA | | CTGGCATCGAGGAGTGACGA | | |
| FURIN_ | GCAGGCAAUU | 2461 | AACCACCCGGACTTGGCAGGCAAT | 2485 | human |
| 769 | AUGAUCCUGG | | TATGATCCTGGGGCCAGTTTT | | |
| FURIN_ | UUUGAUGUCA | 2462 | GATCCTGGGGCCAGTTTTGATGTCA | 2486 | human |
| 796 | AUGACCAGGA | | ATGACCAGGACCCTGACCCC | | |
| FURIN_ | GAGGCCAAUA | 2463 | ATTGCTCTCACCCTGGAGGCCAAT | 2487 | human |
| 1426 | AGAACCUCAC | | AAGAACCTCACATGGCGGGAC | | |
| FURIN_ | UUCAUGACAA | 2464 | TTTAATGACTGGGCCTTCATGACAA | 2488 | human |
| 1876 | CUCAUUCCUG | | CTCATTCCTGGGATGAGGAT | | |
| FURIN_ | UGGGACGCUG | 2465 | CGAAGCCAACAACTATGGGACGCT | 2489 | human |
| 1959 | ACCAAGUUCA | | GACCAAGTTCACCCTCGTACT | | |
| FURIN_ | UGACCAAGUU | 2466 | ACAACTATGGGACGCTGACCAAGT | 2490 | human |
| 1967 | CACCCUCGUA | | TCACCCTCGTACTCTATGGCA | | |
| FURIN_ | UUUUAAUUCA | 2467 | CCCTCCTTGGGCACTTTTTAATTCA | 2491 | human |
| 2711 | CCAAAGUAUU | | CCAAAGTATTTTTTTATCTT | | |
| FURIN_ | UUUAAUUCAC | 2468 | CCTCCTTGGGCACTTTTTAATTCAC | 2492 | human |
| 2712 | CAAAGUAUUU | | CAAAGTATTTTTTTATCTTG | | |
| FURIN_ | UGUUUGAGGA | 2469 | GGGGATCTCAGGGGCTGTTTGAGG | 2493 | human |
| 3524 | UAUAUUUUCA | | ATATATTTTCACTTTGTGATT | | |
| FURIN_ | UUUGAGGAUA | 2470 | GGATCTCAGGGGCTGTTTGAGGAT | 2494 | human |
| 3526 | UAUUUUCACU | | ATATTTTCACTTTGTGATTAT | | |
| FURIN_ | UGAGGAUAUA | 2471 | ATCTCAGGGGCTGTTTGAGGATAT | 2495 | human |
| 3528 | UUUUCACUUU | | ATTTTCACTTTGTGATTATTT | | |
| FURIN_ | UUUCACUUUG | 2472 | TGTTTGAGGATATATTTTCACTTTG | 2496 | human |
| 3539 | UGAUUAUUUC | | TGATTATTTCACTTTAGATG | | |
| FURIN_ | UAUUUCACUU | 2473 | TTTTCACTTTGTGATTATTTCACTTT | 2497 | human |
| 3553 | UAGAUGCUGA | | AGATGCTGATGATTTGTTT | | |
| FURIN_ | UUAGAUGCUG | 2474 | TGTGATTATTTCACTTTAGATGCTG | 2498 | human |
| 3562 | AUGAUUUGUU | | ATGATTTGTTTTTGTATTTT | | |
| FURIN_ | CUGGUUUUGU | 2475 | TCGTGGCCAGCCCGGCTGGTTTTGT | 2499 | human |
| 4101 | AAGAUGCUGG | | AAGATGCTGGGTTGGTGCAC | | |
| FURIN_ | UGGGUUGGUG | 2476 | GGTTTTGTAAGATGCTGGGTTGGTG | 2500 | human |
| 4118 | CACAGUGAUU | | CACAGTGATTTTTTTCTTGT | | |
| FURIN_ | CUUGUAAUUU | 2477 | CACAGTGATTTTTTTCTTGTAATTT | 2501 | human |
| 4143 | AAACAGGCCC | | AAACAGGCCCAGCATTGCTG | | |
| FURIN_ | UUAAACAGGC | 2478 | TTTTTTTCTTGTAATTTAAACAGGC | 2502 | human |
| 4151 | CCAGCAUUGC | | CCAGCATTGCTGGTTCTATT | | |
| FURIN_ | CCCAGCAUUG | 2479 | TGTAATTTAAACAGGCCCAGCATT | 2503 | human |
| 4160 | CUGGUUCUAU | | GCTGGTTCTATTTAATGGACA | | |
| FURIN_ | UGAGAUAAUG | 2480 | TCTATTTAATGGACATGAGATAAT | 2504 | human |
| 4190 | UUAGAGGUUU | | GTTAGAGGTTTTAAAGTGATT | | |
| FURIN_ | GAGAUAAUGU | 2481 | CTATTTAATGGACATGAGATAATG | 2505 | human |
| 4191 | UAGAGGUUUU | | TTAGAGGTTTTAAAGTGATTA | | |
| FURIN_ | UUAGAGGUUU | 2482 | GGACATGAGATAATGTTAGAGGTT | 2506 | human |
| 4200 | UAAAGUGAUU | | TTAAAGTGATTAAACGTGCAG | | |

TABLE 12E
Host targets screened - IL-6R-20 nucleotide targets and 45 nucleotide
gene target regions

| Se- | | SEQ | | SEQ | |
|---|---|---|---|---|---|
| quence | | ID | | ID | |
| ID | Sequence | NO: | Gene Region | NO: | Species |
| IL6R_ | UGAGUCAUGUG | 2507 | GACCGTCCGCCGCTCTGAGTCAT | 2529 | human |
| 38 | CGAGUGGGA | | GTGCGAGTGGGAAGTCGCACTG | | |
| IL6R_ | AGAGCCGGAAG | 2508 | GACCTGCCCGGGGGTAGAGCCG | 2530 | human |
| 437 | ACAAUGCCA | | GAAGACAATGCCACTGTTCACTG | | |
| IL6R_ | CUCAGCAAUGU | 2509 | TTCCGGAAGAGCCCCCTCAGCAA | 2531 | human |
| 666 | UGUUUGUGA | | TGTTGTTTGTGAGTGGGGTCCT | | |
| IL6R_ | GCUCUUGGUGA | 2510 | GACGACAAAGGCTGTGCTCTTGG | 2532 | human |
| 728 | GGAAGUUUC | | TGAGGAAGTTTCAGAACAGTCC | | |
| IL6R_ | AGUGUCGGGAG | 2511 | ATGTGCGTCGCCAGTAGTGTCGG | 2533 | human |
| 876 | CAAGUUCAG | | GAGCAAGTTCAGCAAAACTCAA | | |
| IL6R_ | UUCAGCAAAAC | 2512 | AGTGTCGGGAGCAAGTTCAGCA | 2534 | human |
| 891 | UCAAACCUU | | AAACTCAAACCTTTCAGGGTTGT | | |
| IL6R_ | UCAAACCUUUCA | 2513 | CAAGTTCAGCAAAACTCAAACCT | 2535 | human |
| 902 | GGGUUGUG | | TTCAGGGTTGTGGAATCTTGCA | | |
| IL6R_ | UUCAGGGUUGU | 2514 | GCAAAACTCAAACCTTTCAGGGT | 2536 | human |
| 910 | GGAAUCUUG | | TGTGGAATCTTGCAGCCTGATC | | |
| IL6R_ | CACUCCUGGAAC | 2515 | ACCTGGCAAGACCCCCACTCCTG | 2537 | human |
| 1011 | UCAUCUUU | | GAACTCATCTTTCTACAGACTA | | |
| IL6R_ | ACUCCUGGAACU | 2516 | CCTGGCAAGACCCCCACTCCTGG | 2538 | human |
| 1012 | CAUCUUUC | | AACTCATCTTTCTACAGACTAC | | |
| IL6R_ | GGUUUGAGCUC | 2517 | CTTTCTACAGACTACGGTTTGAG | 2539 | human |
| 1042 | AGAUAUCGG | | CTCAGATATCGGGCTGAACGGT | | |
| IL6R_ | UCAGAUAUCGG | 2518 | GACTACGGTTTGAGCTCAGATAT | 2540 | human |
| 1051 | GCUGAACGG | | CGGGCTGAACGGTCAAAGACAT | | |
| IL6R_ | CUGAACGGUCA | 2519 | AGCTCAGATATCGGGCTGAACG | 2541 | human |
| 1063 | AAGACAUUC | | GTCAAAGACATTCACAACATGGA | | |
| IL6R_ | AAAGACAUUCA | 2520 | TCGGGCTGAACGGTCAAAGACA | 2542 | human |
| 1073 | CAACAUGGA | | TTCACAACATGGATGGTCAAGGA | | |
| IL6R_ | UCCAGCAUCACU | 2521 | GGATGGTCAAGGACCTCCAGCA | 2543 | human |
| 1105 | GUGUCAUC | | TCACTGTGTCATCCACGACGCCT | | |
| IL6R_ | UUGGACAGAAG | 2522 | GGCCATGGGCACGCCTTGGACA | 2544 | human |
| 1229 | GUCUCCUGA | | GAAGGTCTCCTGAGAGGGTCACT | | |
| IL6R_ | CCUGAGAGGGU | 2523 | TTGGACAGAAGGTCTCCTGAGA | 2545 | human |
| 1244 | CACUGCAAA | | GGGTCACTGCAAAAGAGAATCTC | | |
| IL6R_ | CACUGCAAAAG | 2524 | GTCTCCTGAGAGGGTCACTGCAA | 2546 | human |
| 1255 | AGAAUCUCG | | AAGAGAATCTCGTTCCAACCTC | | |
| IL6R_ | UGCAAAAGAGA | 2525 | TCCTGAGAGGGTCACTGCAAAA | 2547 | human |
| 1258 | AUCUCGUUC | | GAGAATCTCGTTCCAACCTCCCT | | |
| IL6R_ | GUGGACCACGCC | 2526 | CCTGTCAATCTGAACGTGGACCA | 2548 | human |
| 1350 | UAAACUAA | | CGCCTAAACTAATTTTTGACTG | | |
| IL6R_ | UGGACCACGCCU | 2527 | CTGTCAATCTGAACGTGGACCAC | 2549 | human |
| 1351 | AAACUAAU | | GCCTAAACTAATTTTTGACTGC | | |
| IL6R_ | UGUGCCAGCUG | 2528 | CTAATTTTTGACTGCTGTGCCAG | 2550 | human |
| 1381 | GAGUGAUGA | | CTGGAGTGATGATAGGCTCACT | | |

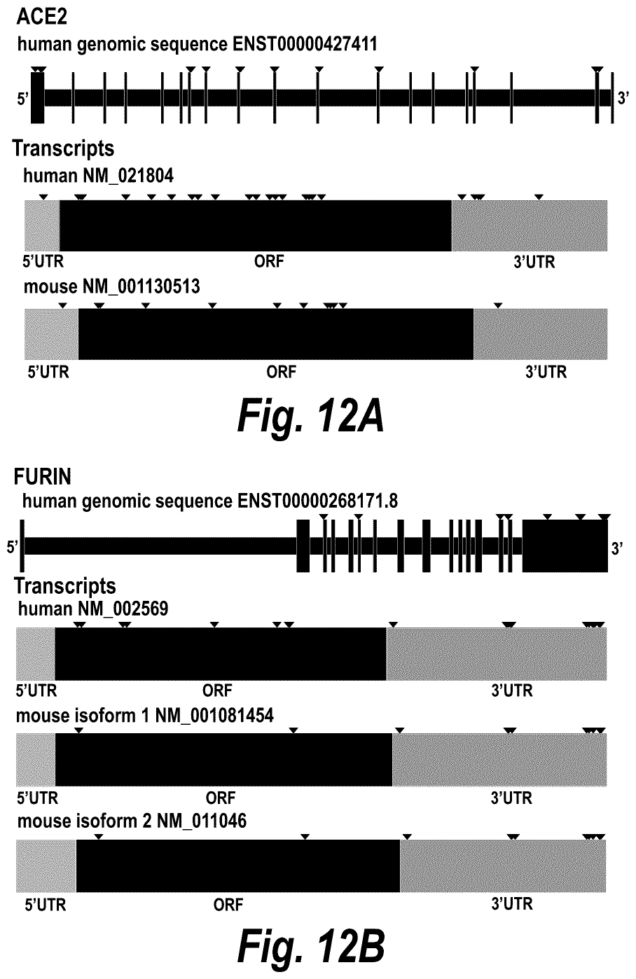

Using a novel algorithm, a panel of siRNAs targeting various regions of ACE2 and FURIN mRNA were designed (FIG. 12). For ASOs, a second step of selection involved testing the secondary structure (accessibility) of the target using the online algorithm lncASO. The sequences of ASOs targeting ACE2 and FURIN are summarized in Table 13A and Table 13B, respectively.

TABLE 13A
ASOs targeting host factors-ACE2 target

| | | SEQ | homology | | |
|---|---|---|---|---|---|
| | Sequence | ID | | | Mon- |
| Oligo ID | (anti sense) | NO: | Human | Mouse | key |
| ACE2_171 | GCAAGTGAACTTTGAT | 2551 | Y | Y | Y |
| ACE2_250 | AAAGGCAGACCATTTG | 2552 | Y | Y | N |
| ACE2_567 | GGCCTCAGCTGCTTGC | 2553 | Y | Y | Y |
| ACE2_694 | GCCGCGGCTGTAGTCA | 2554 | Y | N | N |
| ACE2_702 | ATCAACTGGCCGCGGC | 2555 | Y | N | N |
| ACE2_851 | TACCCCACATATCACC | 2556 | Y | Y | Y |
| ACE2_938 | AGGCCTGGTCCACCAT | 2557 | Y | N | N |
| ACE2_1326 | TCTTCTTGAAAATCGG | 2558 | Y | Y | N |
| ACE2_1425 | AAGACCATCCACCTCC | 2559 | Y | Y | Y |
| ACE2_1533 | GGGTCACAGTATGTTT | 2560 | Y | Y | Y |
| ACE2_1666 | GATGTCACATTTGTGC | 2561 | Y | Y | Y |
| ACE2_2806 | GAGTTCACGGAGGCCC | 2562 | Y | N | N |

TABLE 13B
ASOs targeting host factors-FURIN target

| | | SEQ | homology | | |
|---|---|---|---|---|---|
| | Sequence | ID | | | Mon- |
| Oligo ID | (anti sense) | NO: | Human | Mouse | key |
| FURIN_176 | CCGGGGCTGACTGGTG | 2563 | Y | Y | Y |
| FURIN_450 | AAGATCTGGCCCAGGT | 2564 | Y | Y | N |
| FURIN_963 | GTCACCTCGCCATCCA | 2565 | Y | Y | Y |
| FURIN_1044 | TCATCCTCGGGGCCCC | 2566 | Y | N | Y |
| FURIN_1184 | CGCAGTTGCAGCTGTC | 2567 | Y | N | Y |
| FURIN_1229 | TGGCGCTGCTGATGGA | 2568 | Y | Y | Y |
| FURIN_1400 | TGATGCCGGCTGCTAA | 2569 | Y | N | Y |
| FURIN_1610 | GCTGGGGGGCCACTGT | 2570 | Y | Y | N |
| FURIN_2213 | CACATGAGGCGTGGCA | 2571 | Y | Y | Y |
| FURIN_2217 | GTGGCACATGAGGCGT | 2572 | Y | Y | Y |
| FURIN_2649 | AGGGCGCTCTGGTCTT | 2573 | Y | Y | Y |
| FURIN_2653 | TCAGAGGGCGCTCTGG | 2574 | Y | N | Y |

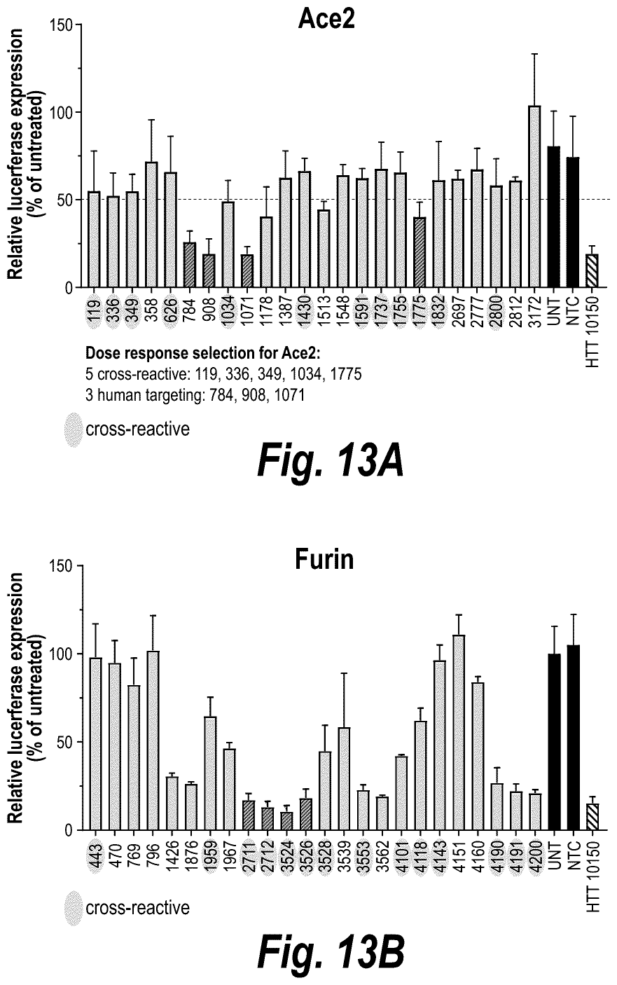

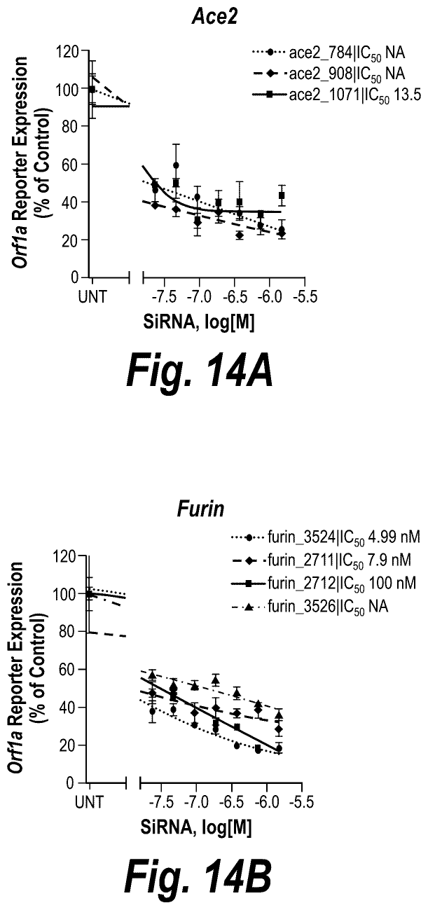

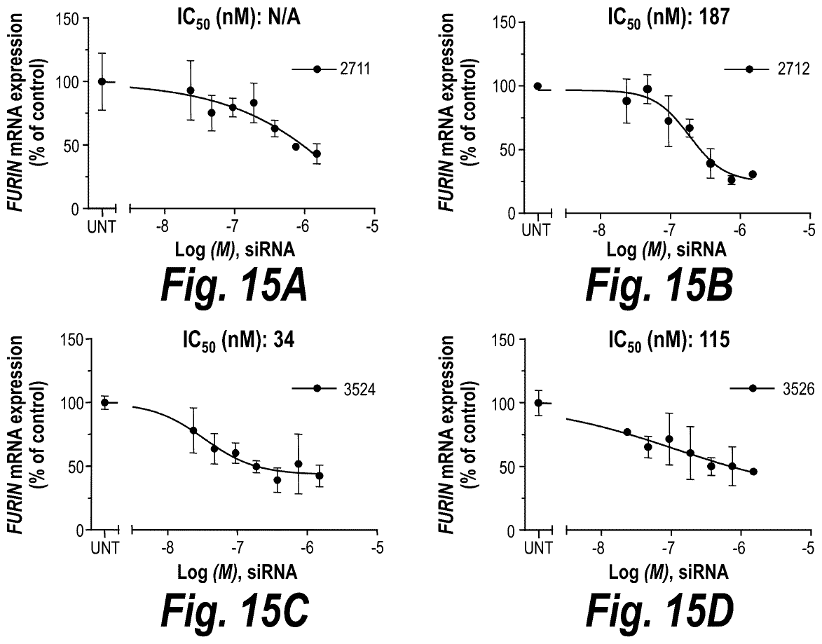

SiRNAs targeting ACE2 and FURIN, two endogenous genes necessary for viral entry and spread were tested for silencing efficacy. FIG. 13A-13B depict the identification of siRNA hits for ACE2 and FURIN, respectively. For each target, at least 3 siRNAs were identified that reduced target mRNA expression below 75% compared to untreated controls. siRNAs were tested in human Hacat cells and silencing was assessed using the QuantiGene assay and confirmed using psicheck reporter system. FIG. 14A-14B depict validation and determination of IC₅₀ values for siRNAs targeting ACE2 (FIG. 14A) and FURIN (FIG. 14B). SiRNAs targeting ACE2 and FURIN were tested for silencing efficacy in 8-point dose response studies. Each siRNA showed potent and efficacious target silencing with IC₅₀ values in the low nanomolar range. FIG. 15A-15D depict validation and determination of IC₅₀ values for four selected siRNAs, tested for silencing efficacy in 8-point dose response studies. Each siRNA showed potent and efficacious target silencing with IC₅₀ values in the low nanomolar range. siRNAs were tested in HaCat cells and silencing was assessed using QuantiGene.

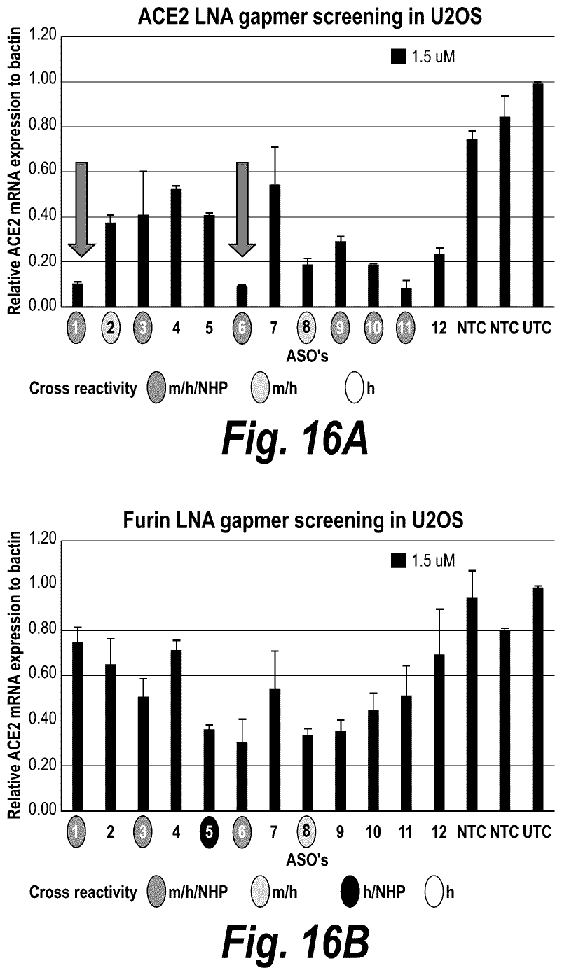

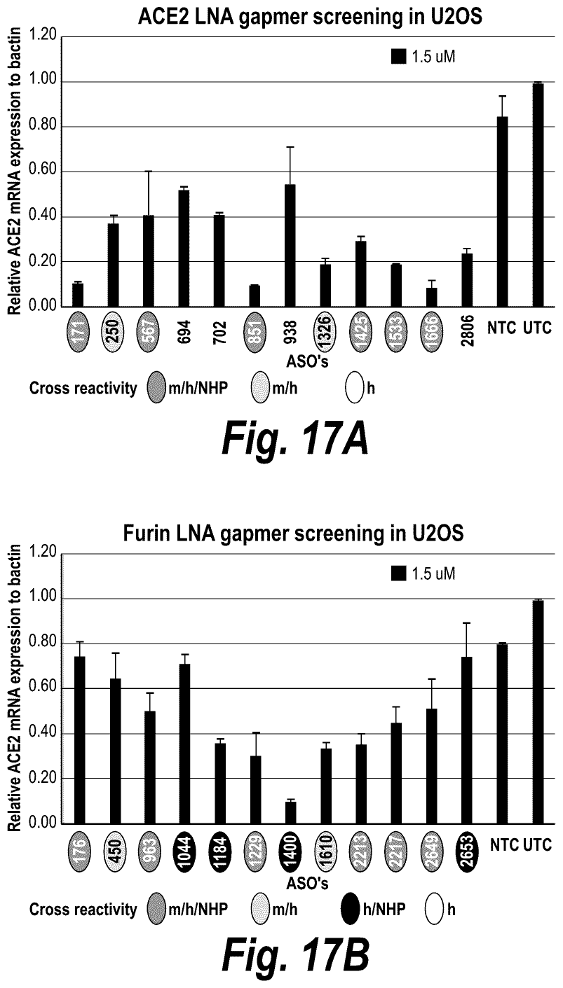

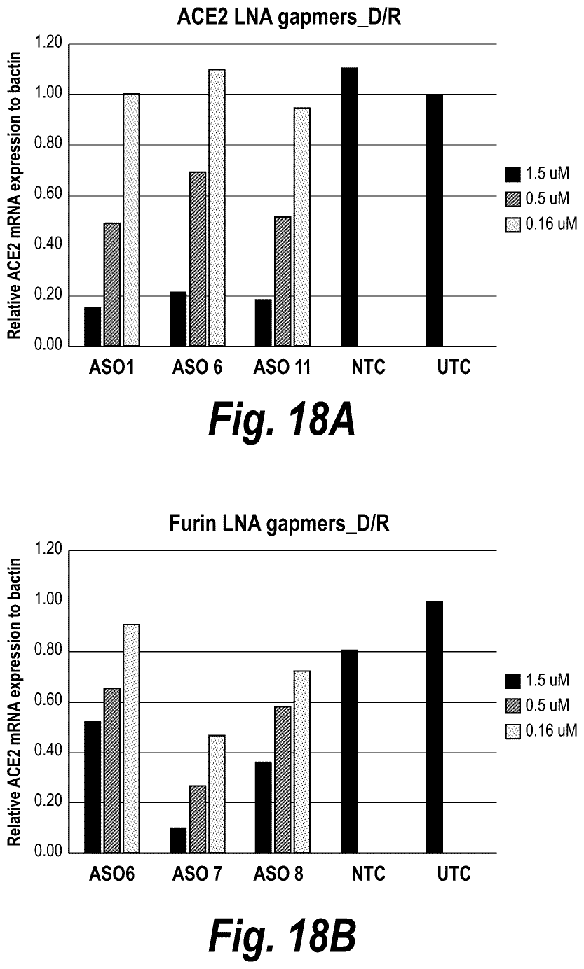

The identification of ASO hits for ACE2 and FURIN are shown in FIG. 16. Twelve LNA gapmers targeting ACE2 (FIG. 16A) and FURIN (FIG. 16B), two endogenous genes necessary for viral entry and spread, were tested for silencing efficacy. For each target, at least 3 ASOs were identified that reduced target mRNA expression below 75% compared to untreated controls. ASOs were tested in human U2OS cells and silencing was assessed using QRT-PCR assay. FIG. 17A-17B depict the identification of ASO hits for ACE2 and FURIN. Twelve LNA gapmers targeting ACE2 (FIG. 17A) and FURIN (FIG. 17B), two endogenous genes necessary for viral entry and spread for silencing efficacy. For each target, we identified at least 3 ASOs that reduced target mRNA expression below 75% compared to untreated controls. ASOs were tested in human U2OS cells and silencing was assessed using QRT-PCR assay. Concentration: 1.5 μM; Time point: 72 hours. FIG. 18 depict validation and determination of IC₅₀ values for ASOs targeting ACE2 (FIG. 18A) and FURIN (FIG. 18B). ASOs targeting ACE2 and FURIN were tested for silencing efficacy in 3-point dose response and gene expression was measured using QRT-PCR. Concentration: Top=1.5 μM; Time point: 5 days.

### Example 3. Delivery to the Lungs

In the present invention, methodologies are disclosed for a uniform and efficient delivery of fully stabilized siRNAs to the lung. Lung delivery was achieved after intratracheal administration (IT). This route might have significant advantages when using siRNA cocktails for prophylaxis and in the field as it is minimally invasive. Efficient delivery is observed to several cell types, including endothelial, epithelial, fibroblasts and immune cells in the lungs. Hydrophobically modified siRNAs often induce strong immune responses after local delivery and thus can't be widely used. Surprisingly therapeutic distribution of Phosphorothioate enriched, fully modified siRNAs was observed after intratracheal administration. Delivery to relevant cell types was observed after administration of both monovalent and divalent compounds, with increased delivery after administration of divalent versus monovalent entities.

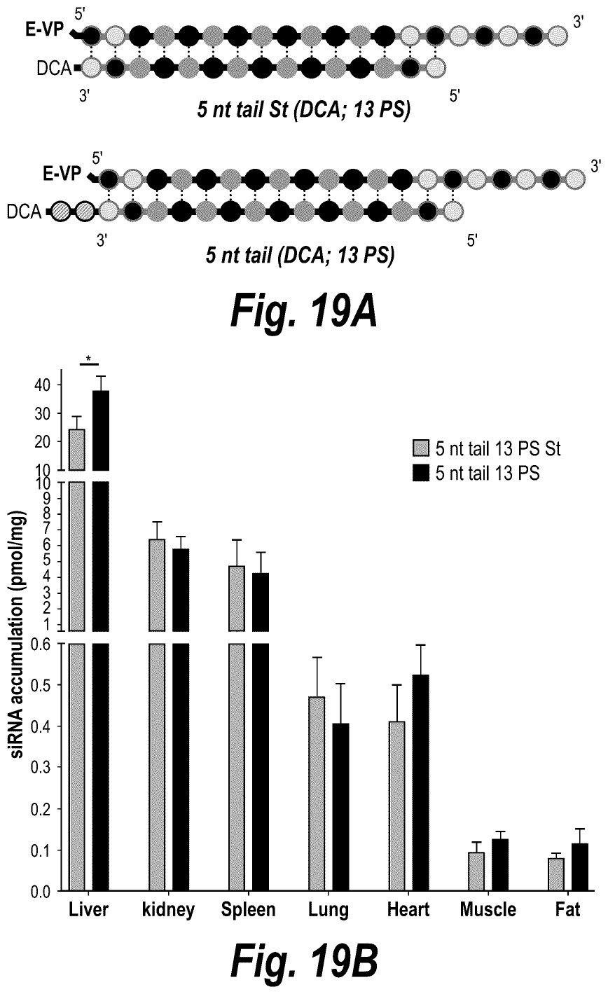

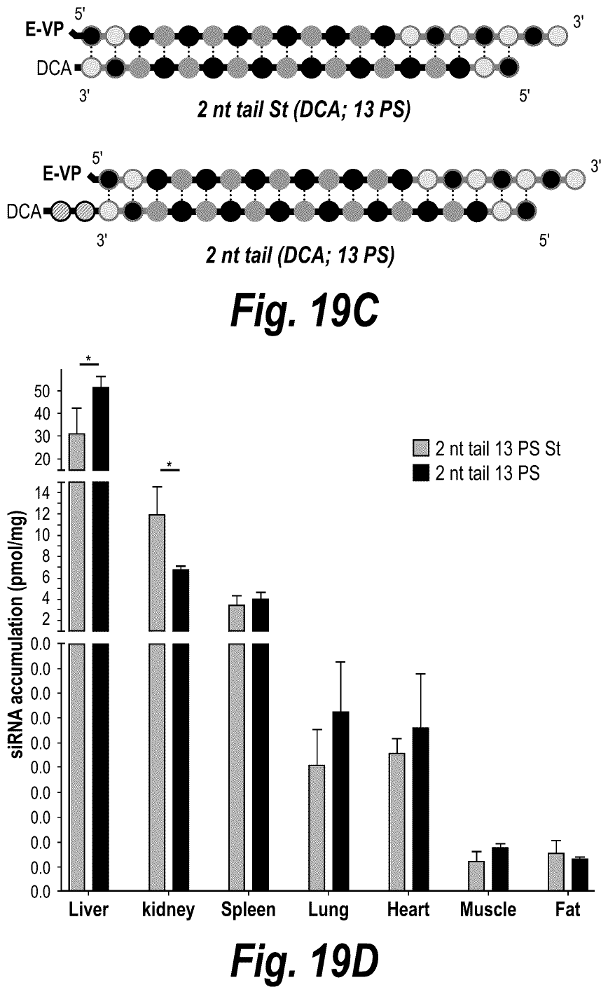

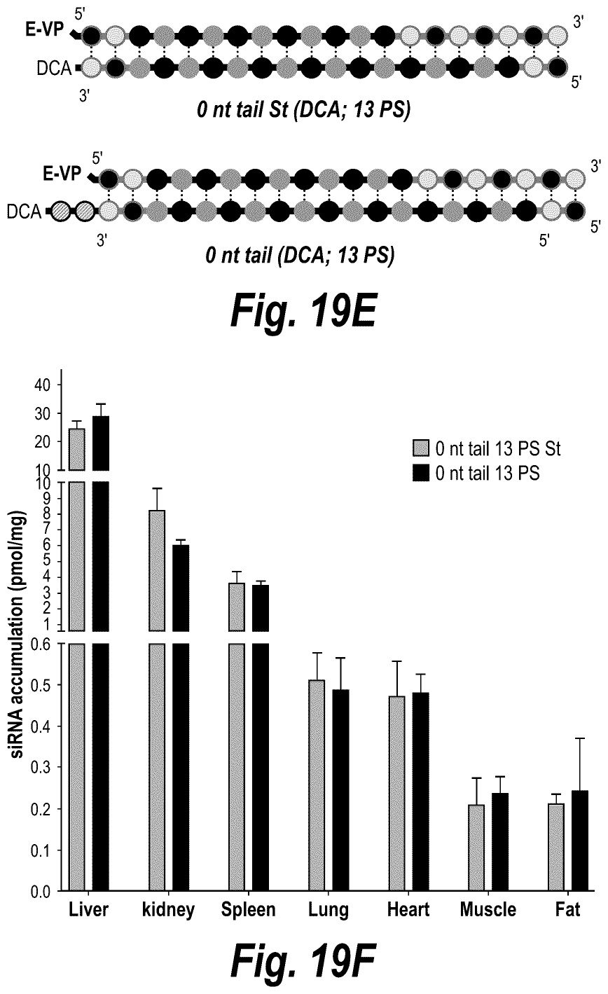

The presence of a two-thymidine linker between the conjugate and the siRNA does not impact siRNA tissue distribution profile (FIG. 19). Three different siRNA structural configurations were studied to evaluate the impact of the nature of the linker on distribution of DCA-conjugated siRNA in liver, kidney, spleen, lung, heart, muscle and fat 1-week after a single SC injection with 20 mg/kg (n=5-6 mice per group±SD), as measured by a PNA hybridization assay. Data show efficient delivery of all three siRNA configurations to the lung.

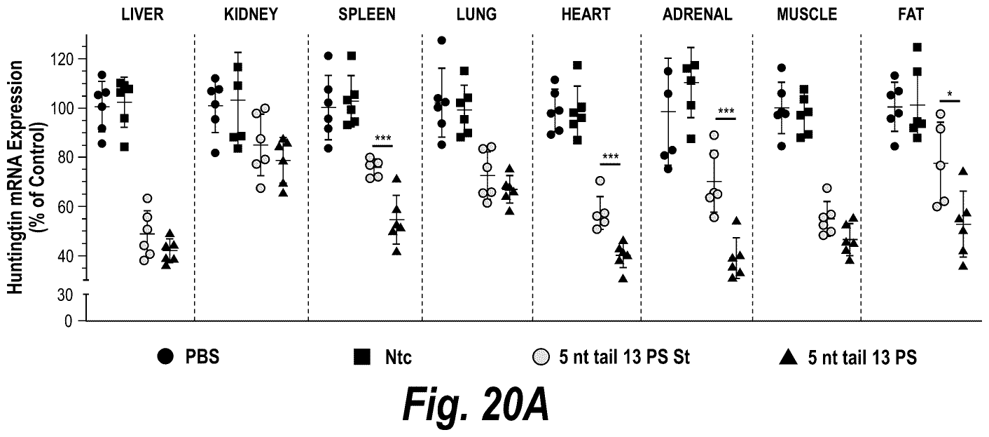

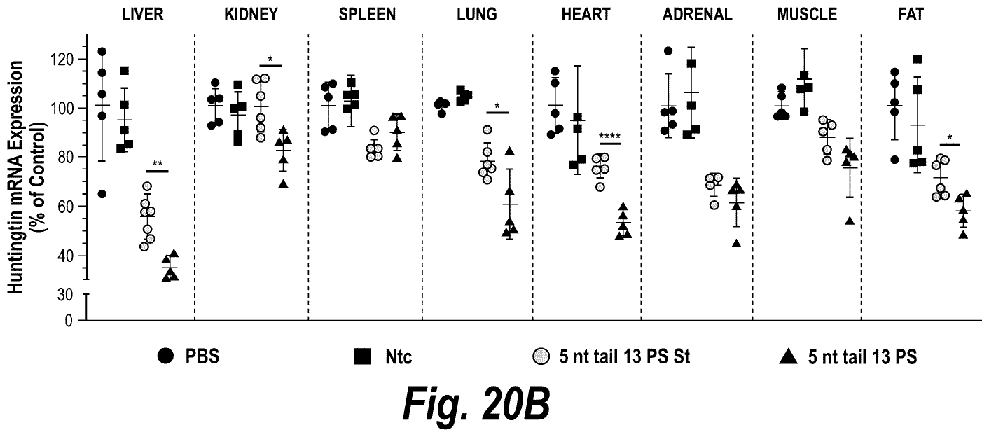

The presence of a two-thymidine linker was found to increase DCA-conjugated siRNA silencing in multiple tissues, as measured by huntingtin and cyclophilin B mRNA expression (FIG. 20). Six mice per group were injected with siRNA by SC injection (FVB/N mice); 20 mg/kg; and tissues were collected one week after injection. The mRNA levels were measured using QuantiGene® (Affymetrix), and normalized to a housekeeping gene, Hprt (Hypoxanthine-guanine phosphoribosyl transferase), and presented as percent of PBS (Phosphate buffered saline) control (mean±SD).

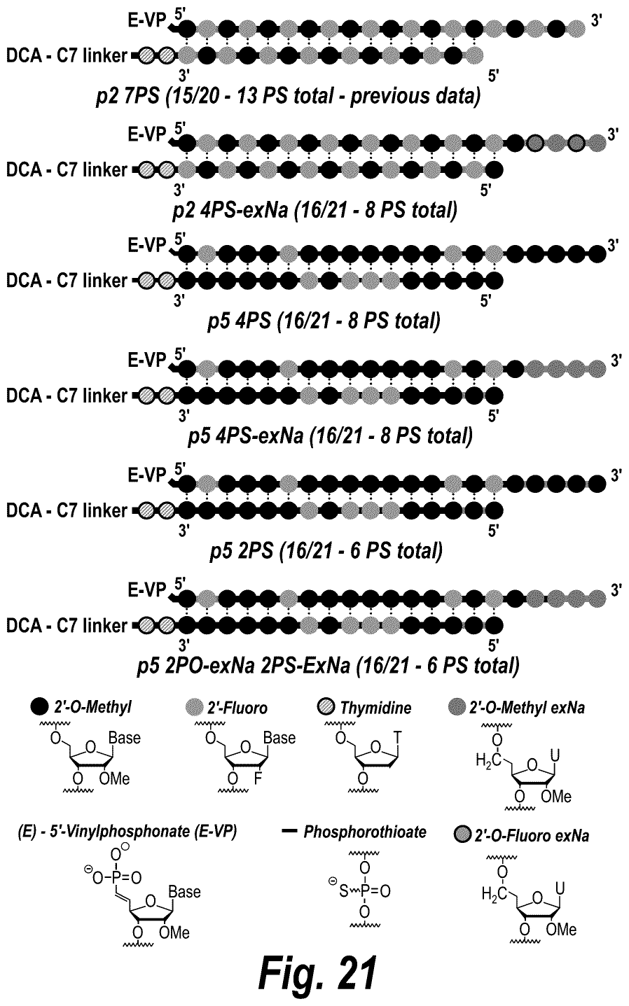

Six siRNAs were then designed containing different numbers of 3′ exNA modifications and phosphorothioates. (FIG. 21) for lung delivery via systemic (SC) delivery. In FIG. 22 the impact of the chemical composition on siRNA distribution and efficacy is evaluated. The data show increased accumulation of DCA-conjugated siRNAs with exNA modifications compared to those without exNAs in all tissues including the lungs. Injections were done SC, 20 mg/kg, in three mice, for 1 week, and distributions were assessed in the PNA hybridization assay. Results show for the p2 scaffold show that 4PS-exNa accumulation was comparable to 7PS in liver, spleen, and lung; 4PS-exNa >7 PS in heart, muscle, fat; 7PS >4PS-exNa in kidney. p5 scaffold: 4PS-exNa >2PO-exNa 2PS-exNa ≥4 PS >2 PS.

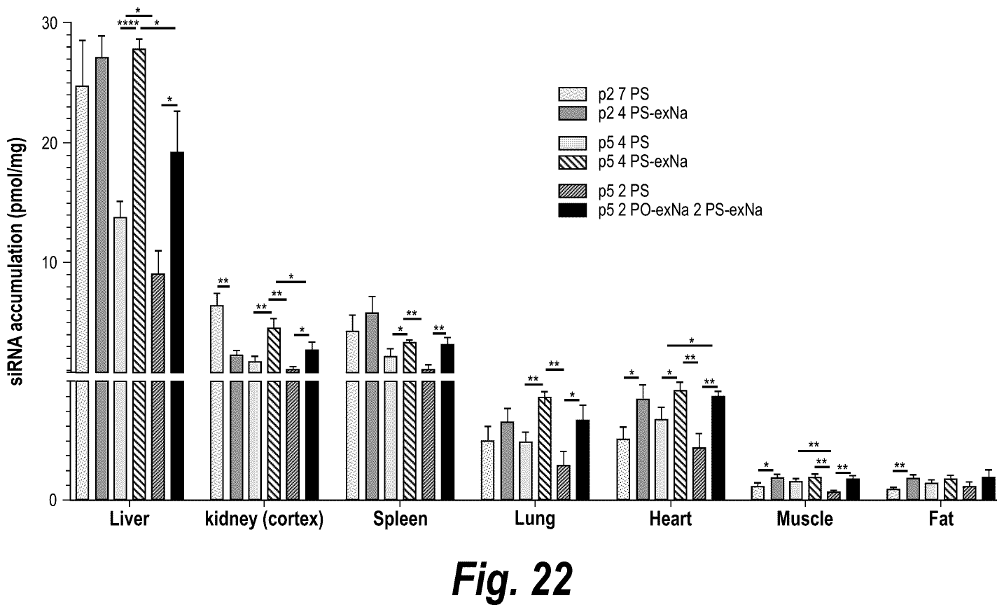

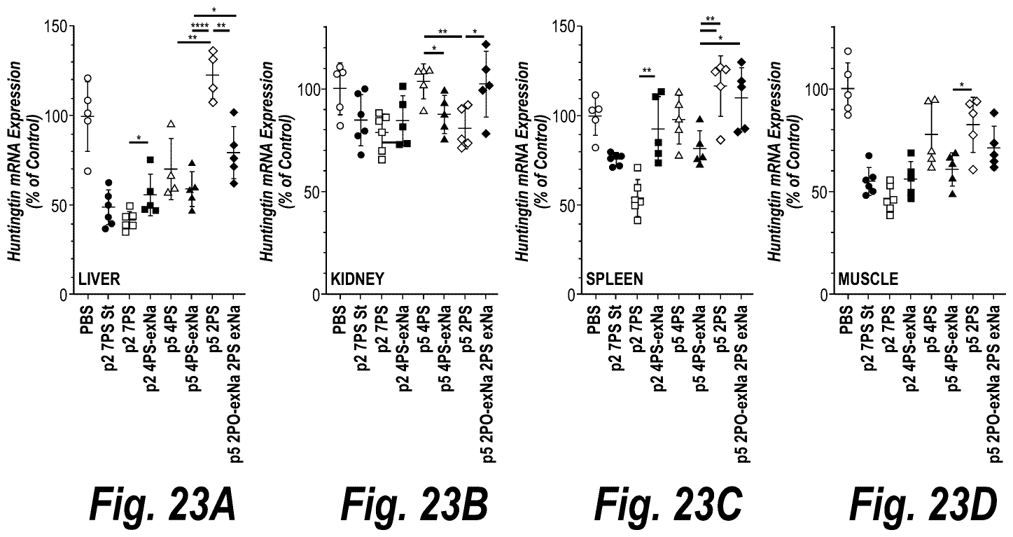

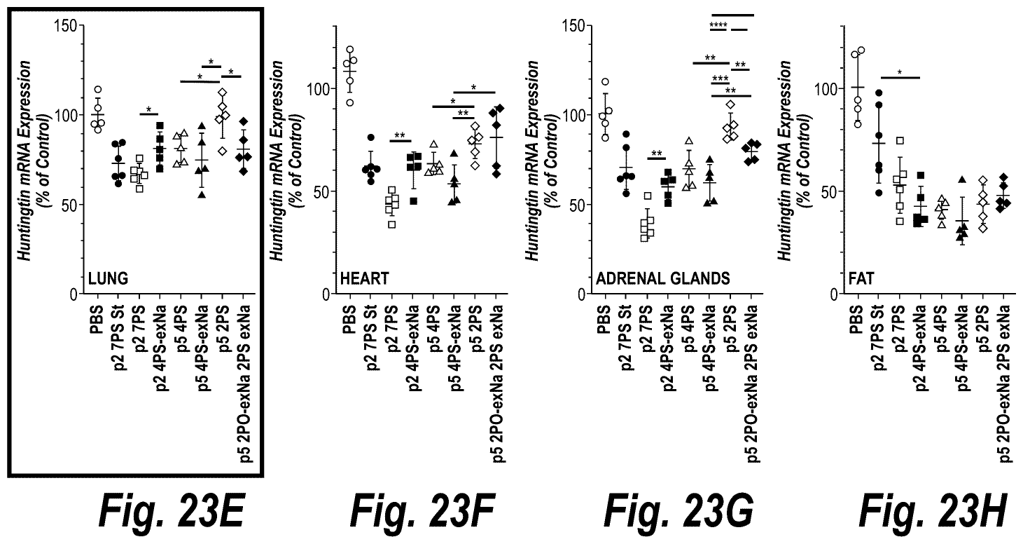

Increased silencing of DCA-conjugated siRNAs was achieved with exNA modifications compared to those without exNAs in all tissues including the lungs (FIG. 23). Target mRNA silencing (Htt) after systemic administration of siRNAs conjugated to DCA and containing different numbers of 3′ exNA modifications and phosphorothioates was assessed in various organ tissues including the liver, kidney, spleen, muscle, lung, heart, adrenal glands, and fat. The delivery was done by SC injection, 20 mg/kg, in five mice per group for one week, and quantification was done with the bDNA QuantiGene assay.

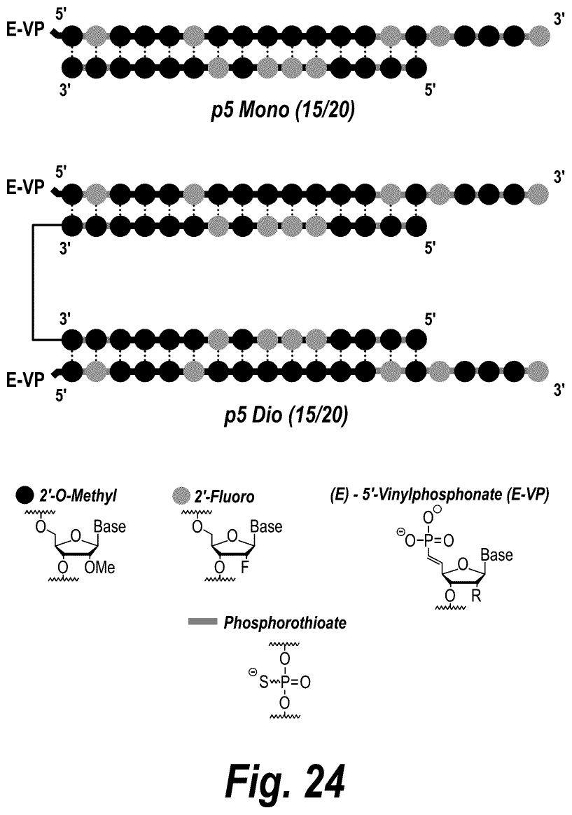

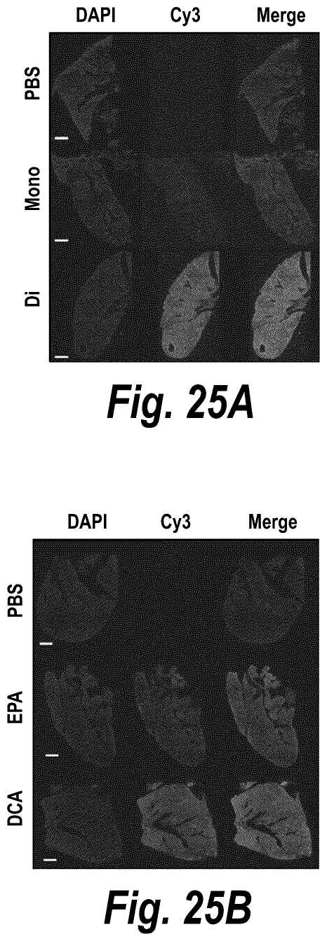

SiRNAs were then designed for lung delivery, to evaluate the impact of the chemical composition on siRNA distribution and efficacy (FIG. 24). The distribution and delivery throughout the lung of mono- and divalent siRNAs (Cy-3) is shown in FIG. 25A, and of DCA and EPA conjugated siRNAs (Cy-3) in FIG. 25B. Delivery via intratracheal injection (mono and divalent conjugates) were at 20 nmol for monovalent, and 40 nmol for divalent siRNAs, in two mice per group, and tissues were harvested after 24 h. Subcutaneous delivery (EPA and DCA conjugates) was at 40 nmol per construct in three mice per group and tissues were harvested after 48 h. Magnification is 5×, and the scale=1 mm.

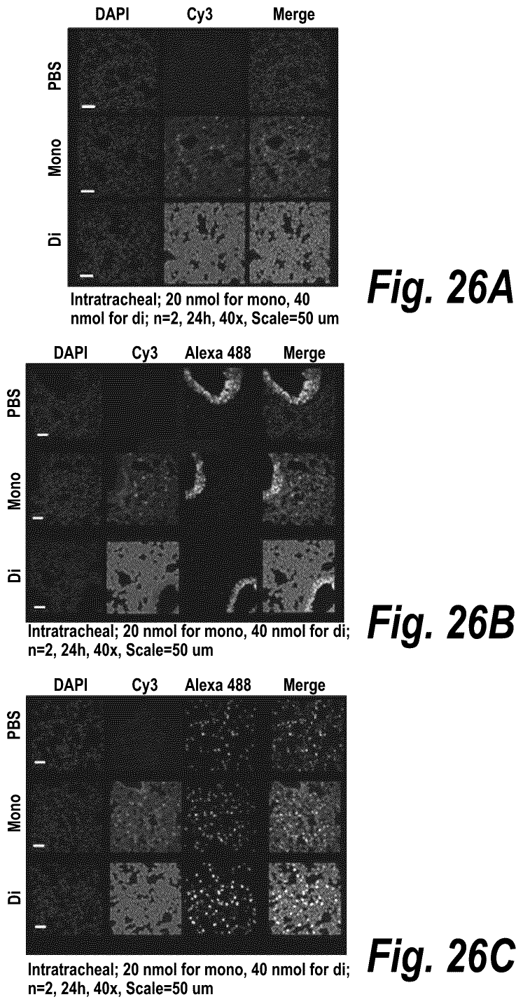

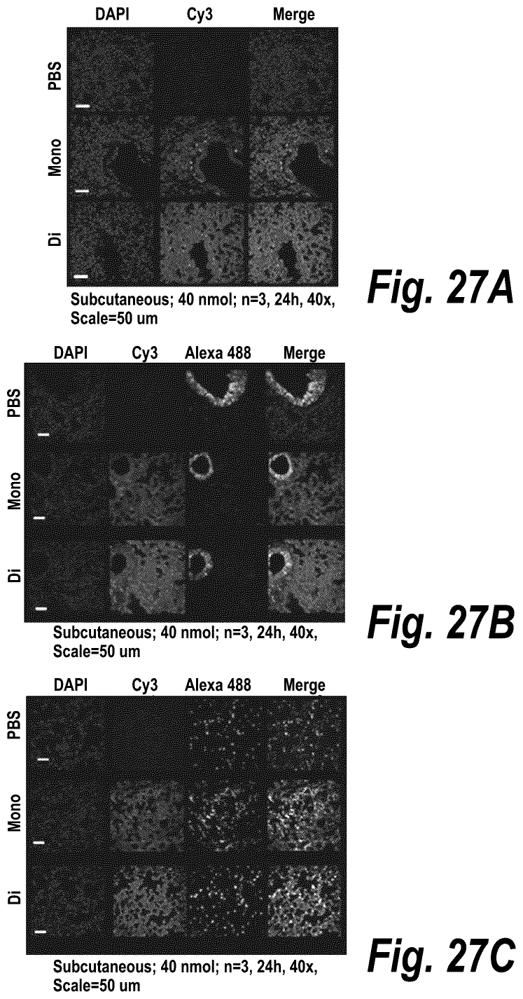

Divalent siRNAs distribute to all cells of the lungs and saturate both alveolar and epithelial (club) cells 24 hours after intratracheal administration. Accumulation of mono and di-valent siRNA (Cy-3, red) after intratracheal administration is shown in FIG. 26. Distribution is shown throughout the lung (FIG. 26A), in club cells (FIG. 26B; green), and in alveoli type II cells (FIG. 26C; green) as compared to PBS controls. FIG. 27 depict results obtained with EPA and DCA conjugates delivered by subcutaneous (SC) administration as assessed after 48 h.

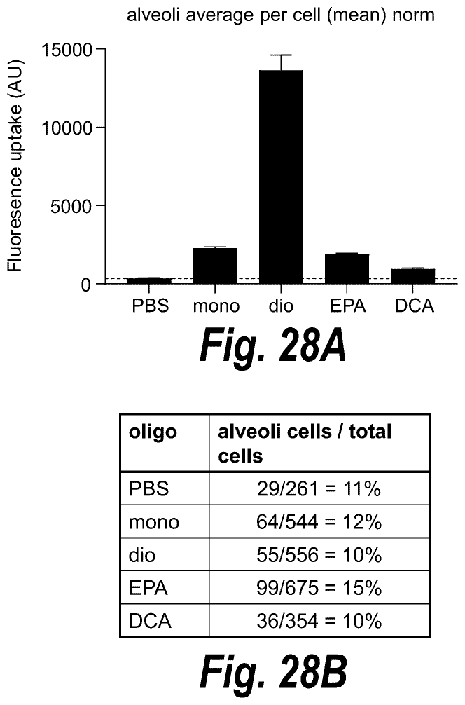

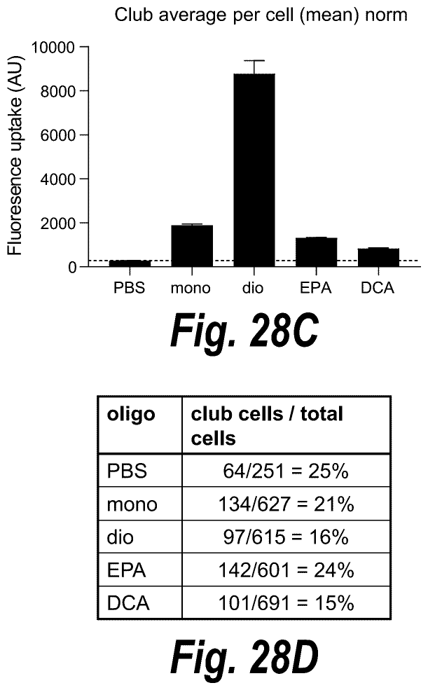

Divalent siRNAs showed the highest amount of uptake among mon-divalent, EPA-conjugated and DCA-conjugated siRNA's, both in alveolar cells as well as in club cells (FIG. 28A and FIG. 28C, respectively, as quantitated in FIG. 28B. and FIG. 28D, respectively). Quantitation was performed using cy3 fluorescence signal intensity and colocalization with markers of different cell types, siRNA accumulation was quantified after systemic (SC) administration of EPA and DCA conjugated siRNAs, and intratracheal administration of mono and di-valent siRNAs. All siRNAs delivered to cells throughout the lung but to different extents.

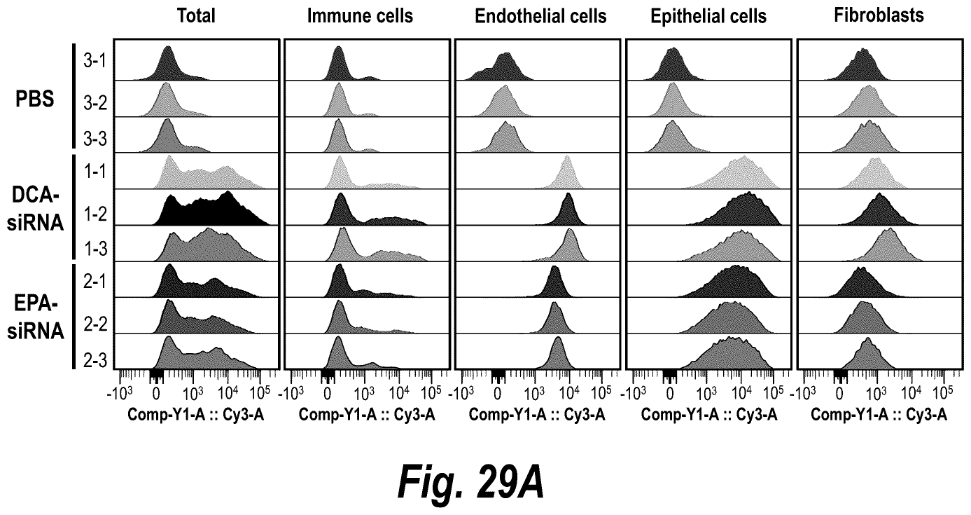

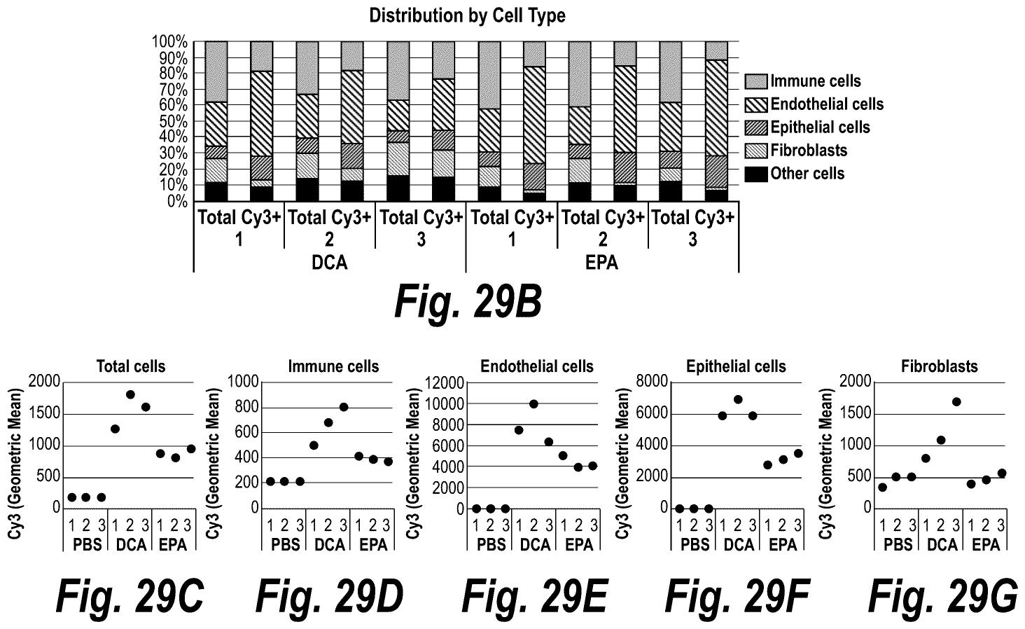

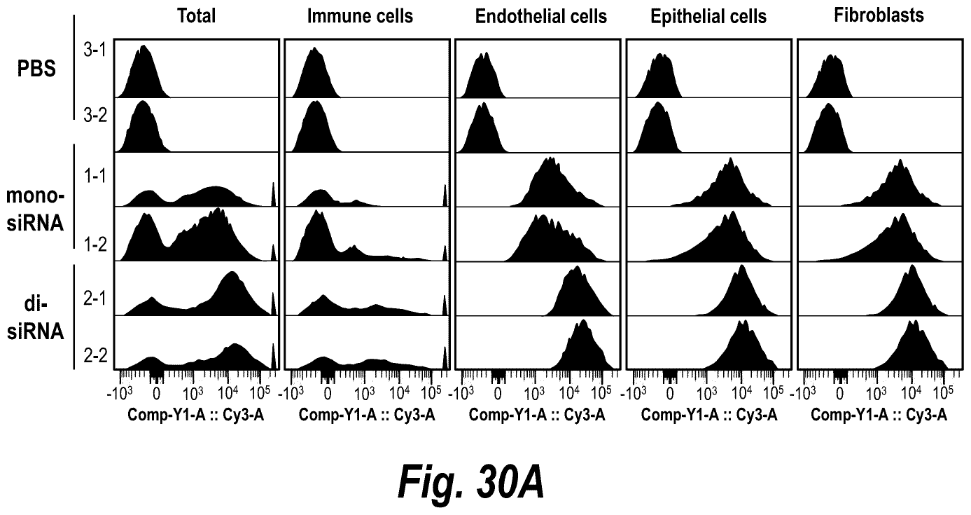

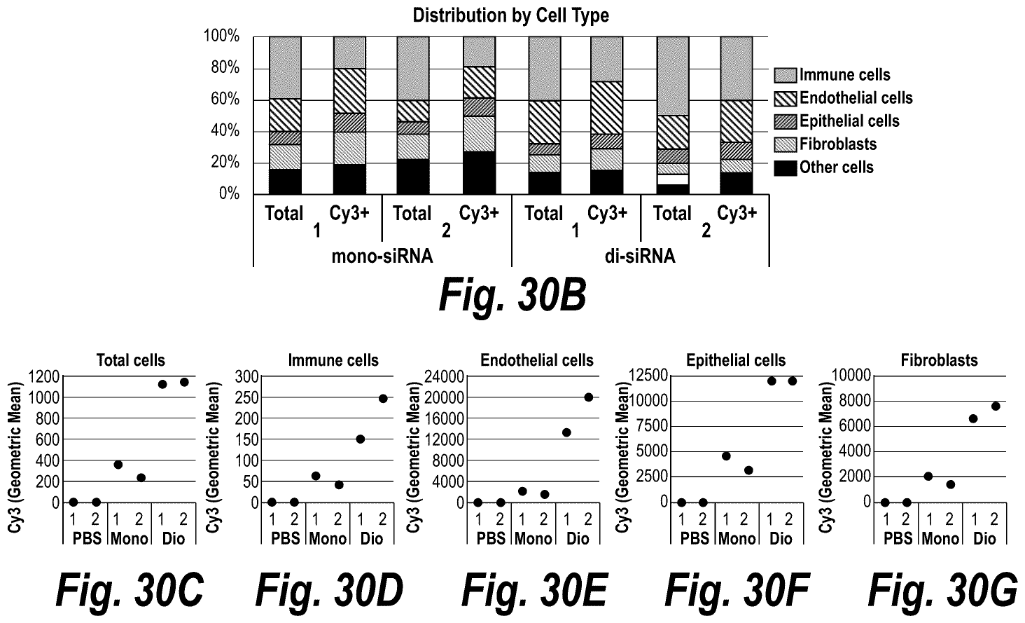

Monovalent, divalent, EPA-conjugated and DCA-conjugated siRNA 1 siRNAs delivered to cells throughout the lung, but to a different extent. Cy-3 signals were quantitated in total cells, immune cells, endothelial cells, epithelial cells and fibroblasts, after systemic (SC) administration of EPA and DCA conjugated siRNAs (FIG. 29A-29C) and after intratracheal (IT) administration of mono- and divalent siRNAs (FIG. 30A-30C).

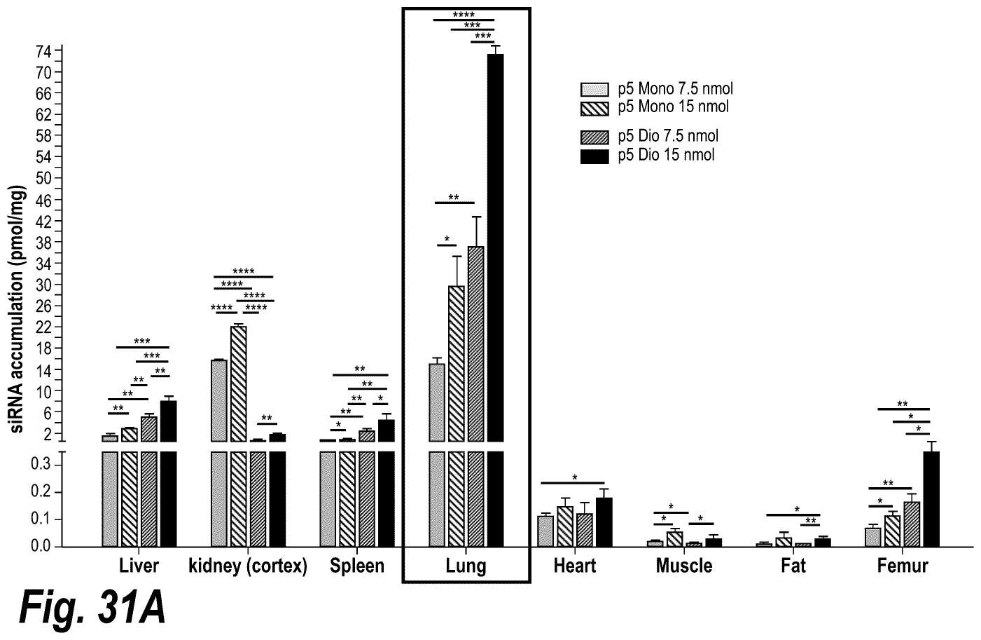

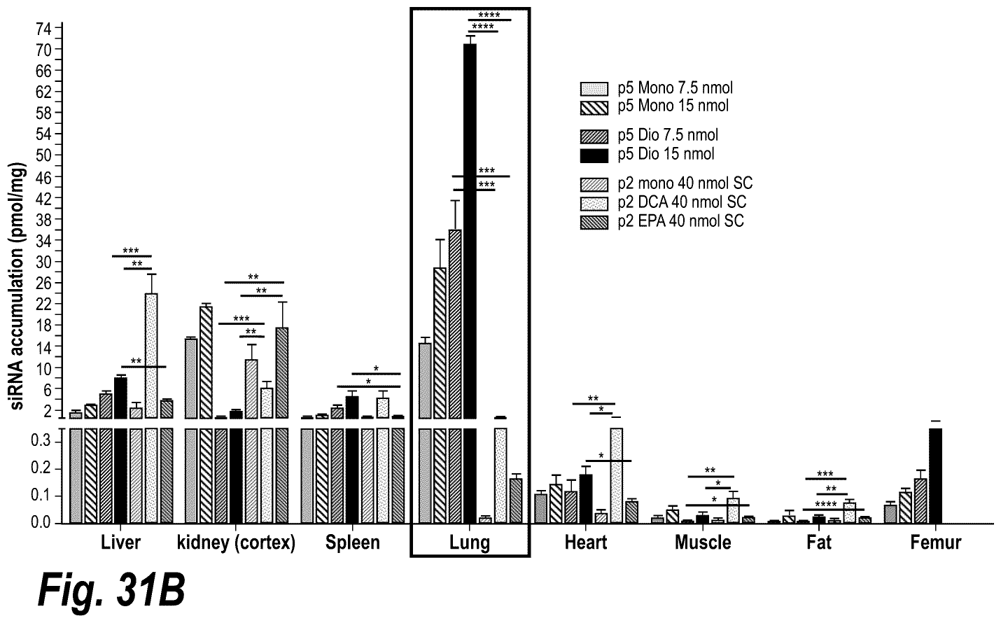

There is a clear increased accumulation of di-siRNA compared mono siRNA or DCA- or EPA-conjugated siRNA in the lungs. The distribution and accumulation of mono and di-siRNAs in various tissues were assessed after intratracheal injection, and for DCA- and EPA-conjugated siRNA after SC injection, as shown in FIG. 31. Amounts injected by intratracheal administration were 7.5 and 15 nmol, for mono- and divalent siRNA, respectively, and 40 nmol for EPA/DCA conjugated siRNA, in groups of three mice each, followed by quantitation of siRNA accumulation after a week using the PNA hybridization assay.

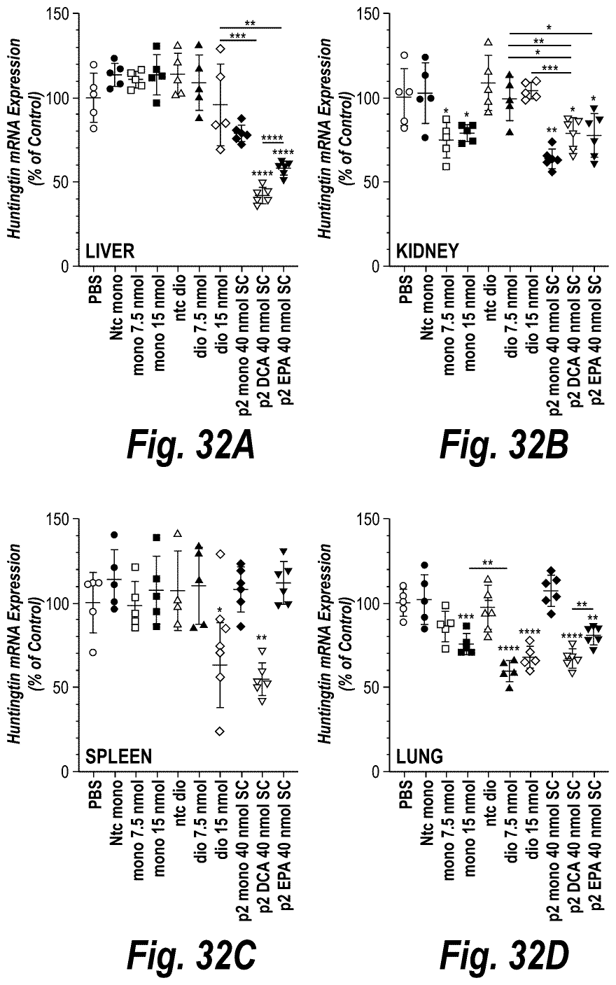

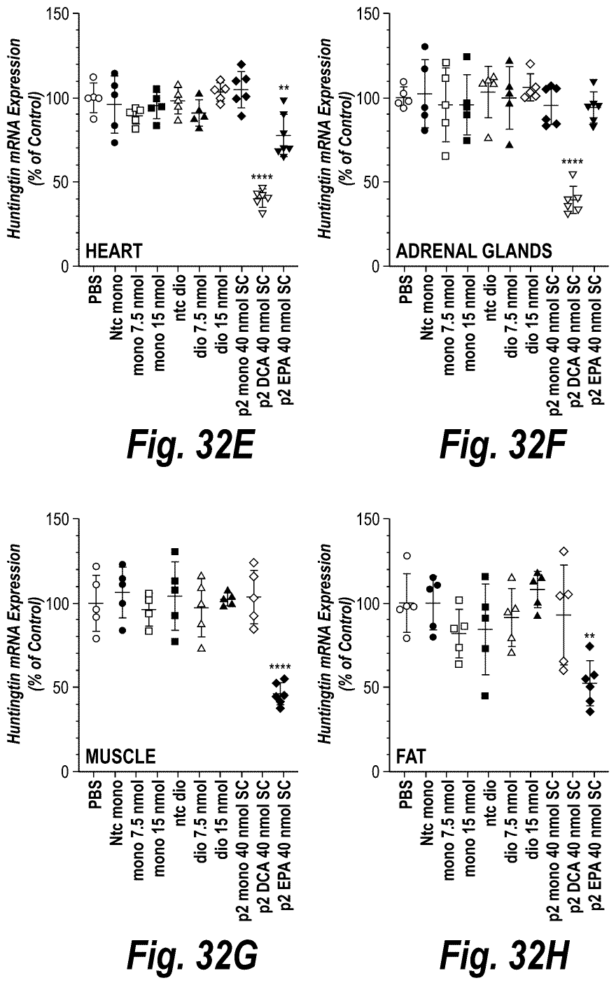

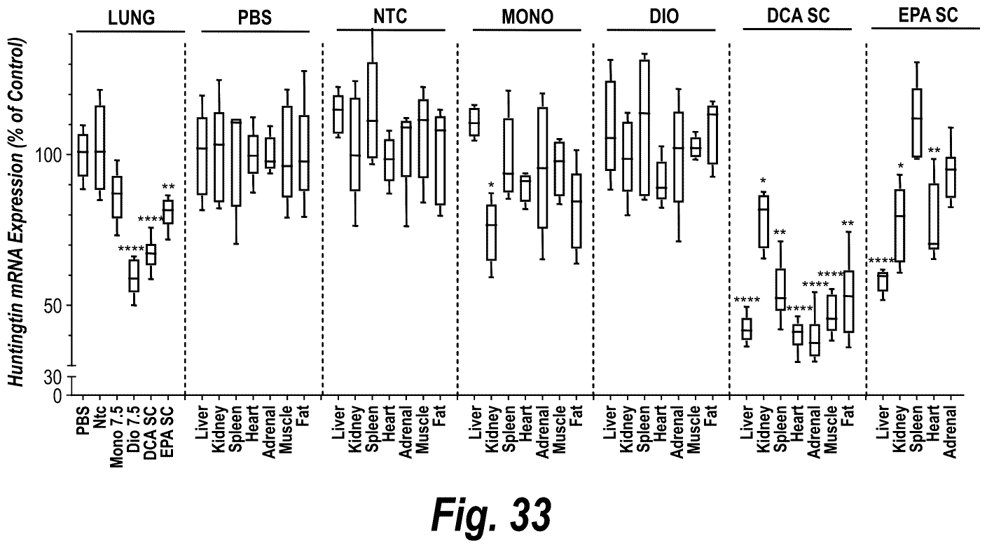

A low dose of di-siRNA achieved the best silencing in lungs without silencing the gene in other tissues. FIG. 32A-32H and FIG. 33 show target mRNA silencing (Htt) after intratracheal administration of mono and di-siRNAs (7.5 or 15 nmol, respectively) in liver, kidney, spleen, lung, heart, adrenal glands; muscle and fat tissues, showing that divalent siRNA selectively silences the Htt mRNA in the lung.

### Example 4. Additional SARS-CoV-2 Target Sites

### Materials and Methods for Example 4

siRNA treatment and infection Assay

siRNAs were complexed with Lipofectamine RNAiMAX (Invitrogen) according to the manufacturer's protocol and added to the wells of a 96-well plate at the desired final concentration (10 nM for screening, concentration range for dose response assays). To each of these wells was added A549 cells expressing the human ACE2 receptor at a final concentration of 15,000 cells/well. The plate was incubated for 36 hours at 37° C., 5% CO₂. After 36 hours of siRNA treatment, SARS CoV-2 virus was added to each well at a final MOI of 0.1 (unless otherwise specified) and incubated for 1 hour. One hour after virus addition, wells were washed with PBS and incubated for a further 48 hours followed by processing for molecular biology analysis.

### Viral RNA Abundance

Viral RNA abundance was measured from the supernatants of cells 48 hours post virus infection. Briefly, 100 μL of cell culture supernatant from cells was treated with Trizol-LS according to the manufacturer's recommendations. RNA was then isolated using standard protocols using chloroform and ethanol. The abundance of SARS-CoV-2 Nucleocapsid RNA was measured by real-time quantitative PCR using the QuantiFast Pathogen RT-PCR kit (Qiagen) with the 2019-nCoV CDC qPCR Probe (IDT).

### Viral Protein Abundance

Viral protein abundance was measured using immunofluorescence staining with anti-SARS-CoV-2 spike antibody to detect viral spike protein. Briefly, cells were fixed using 4% paraformaldehyde and serial ethanol dehydration followed by standard immunofixation procedures to detect proteins. Cells staining positive for the viral spike protein were counted using a fluorescence microscope.

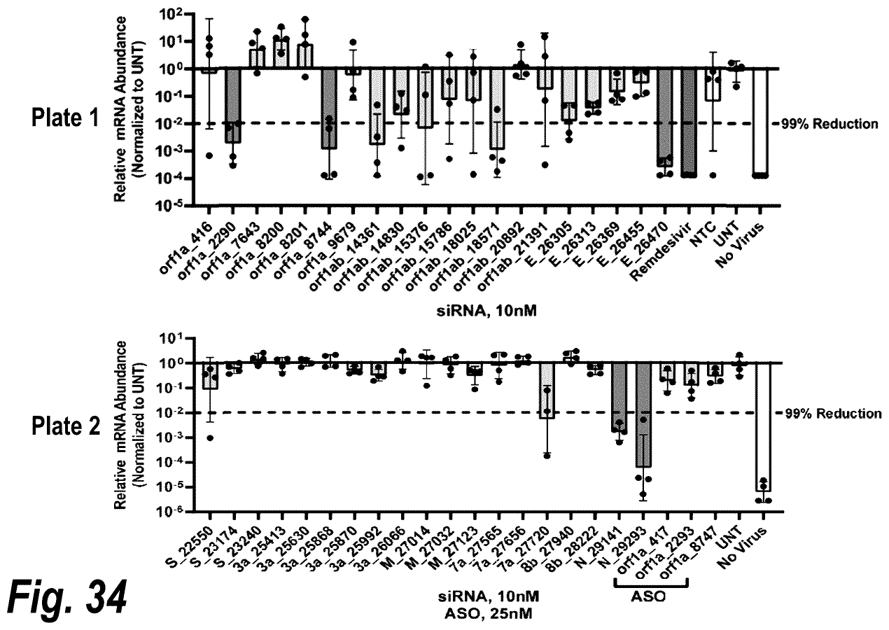

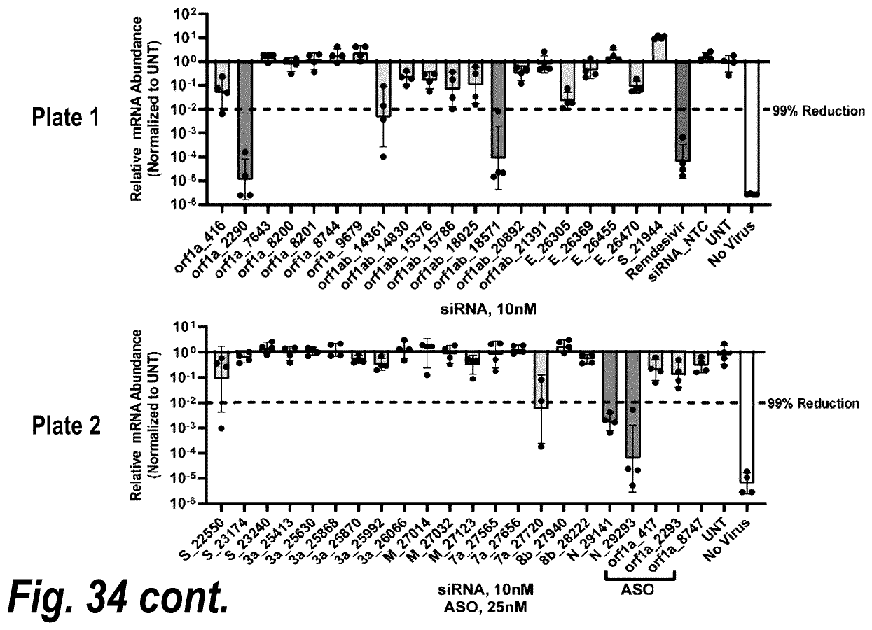

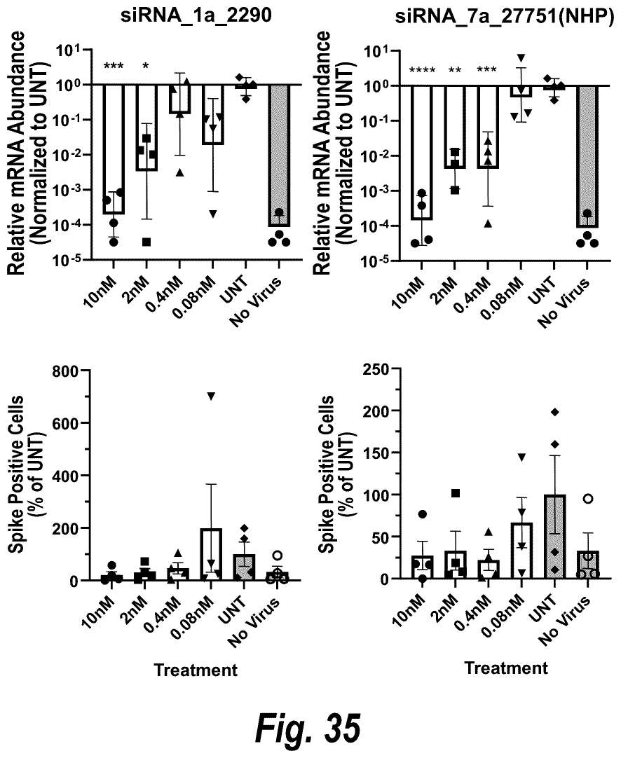

Additional siRNAs and ASOs were tested against various SARS-CoV2 genes. siRNAs and ASOs were tested in A549-ACE2 cells and silencing was assessed using the psi-check reporter system. siRNA concentration: 10 nM; ASO concentration: 25 nM; Time point: 72 hours. As shown in FIG. 34, numerous siRNAs and ASOs were capable of reducing SARS-CoV-2 mRNA levels by 99%, including several that reduce levels to a similar level as remdesivir, an approved therapy for COVID-19.

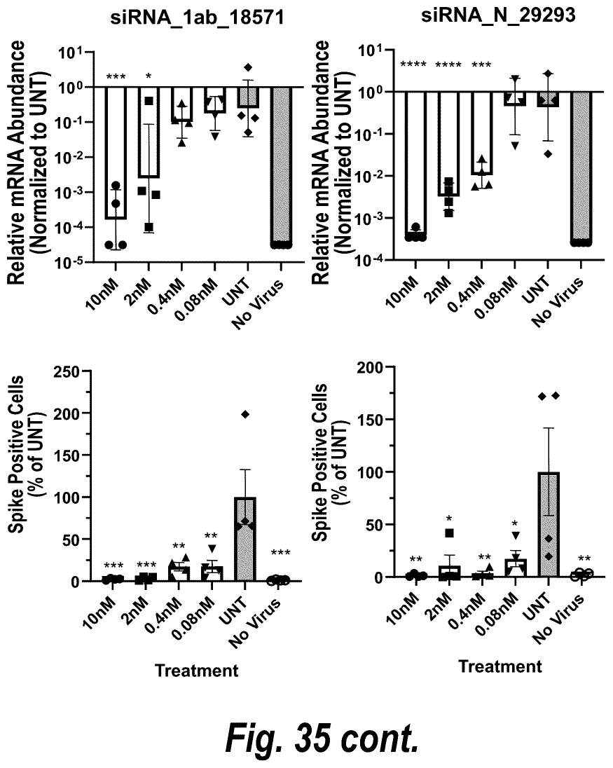

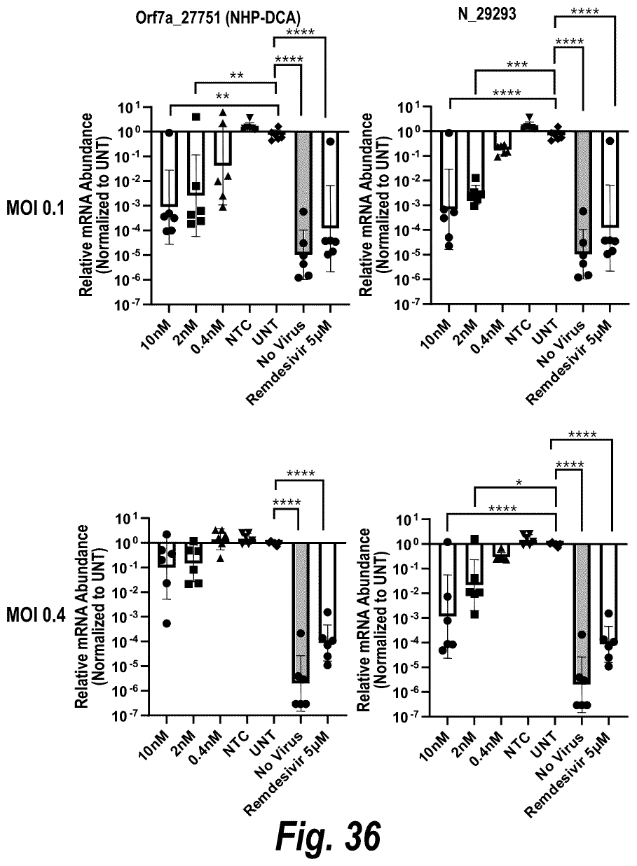

Based on the results of the screen performed in FIG. 34, several top hits were tested in dose response experiments. siRNAs 1a_2290, 7a_27751, 1ab_18571, and N_29293 were each tested at concentrations of 10 nM, 2 nM, 0.4 nM, and 0.08 nM. As shown in FIG. 35, each of the tested siRNAs were able to effectly silence SARS-CoV-2 mRNA at several doses. Moreover, the tested siRNAs led to reduction of SARS-CoV-2 spike protein positive cells.

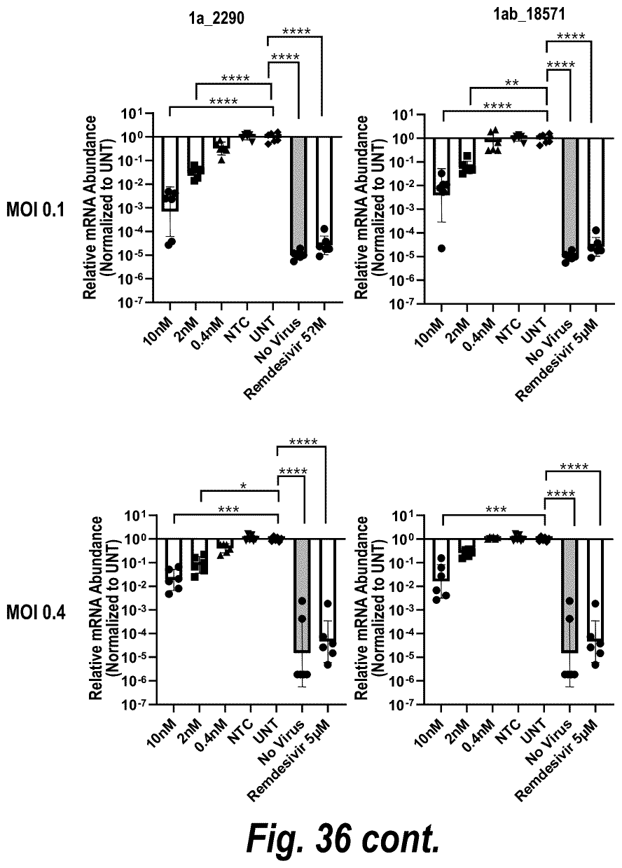

An additional dose response experiment was performed as described above for FIG. 35. In this experiment, cells were infected with SARS-CoV-2 at a multiplicity of infection (MOI) of 0.1 and 0.4. As shown in FIG. 36, the tested siRNAs were able to effectly silence SARS-CoV-2 mRNA at several doses and at both MOIs tested.

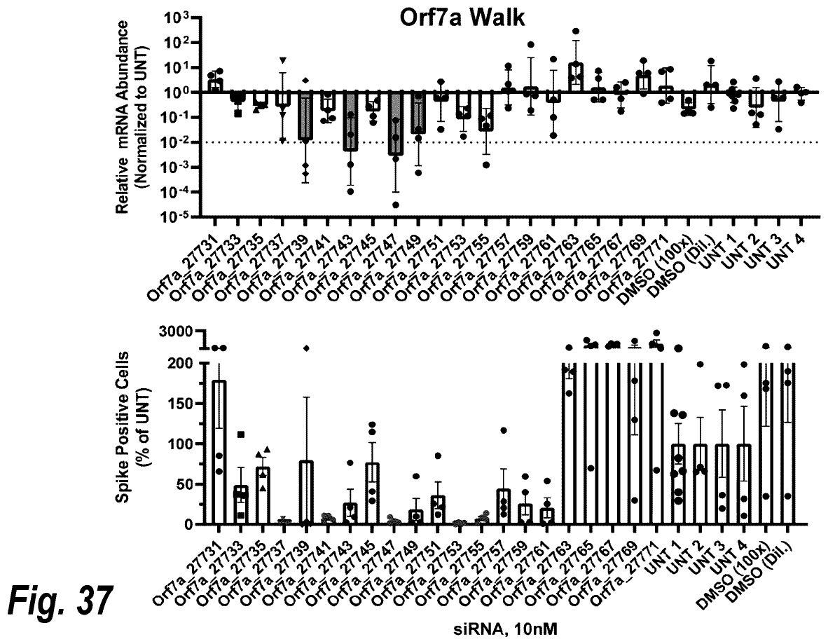

An additional screen of siRNAs was performed targeting the orf7a SARS-CoV2 gene. siRNAs were tested in in A549-ACE2 cells and the data reported was relative mRNA abundance of the targeted orf7a SARS-CoV2 gene and the percent of cells that are postive for the SARS-CoV2 spike protein. siRNA concentration: 10 nM; Time point: 72 hours. As shown in FIG. 37, numerous siRNAs were identified that effectively target orf7a.

The antisense and sense strands of the siRNAs tested in FIGS. 35 and 36 are shown below in Table 14.

TABLE 14
Antisense and Sense sequences of select siRNAs.

SEQ ID NO:

Orf7a_27751

| | | |
|---|---|---|
| Antisense | 5′ UGAAAGUUCAAUCAUUCUUUU 3′ | 2575 |
| Sense | 5′ AAUGAUUGAACUUUCA 3′ | 2576 |

N_29293

| | | |
|---|---|---|
| Antisense | 5′ UGAAAUUUGGAUCUUUGUUUU 3′ | 2577 |
| Sense | 5′ AAAGAUCCAAAUUUCA 3′ | 2578 |

Orf1a_2290

| | | |
|---|---|---|
| Antisense | 5′ UAAGCUUAAAGAAUGUCUUUU 3′ | 2579 |
| Sense | 5′ ACAUUCUUUAAGCUUA 3′ | 2580 |

Orf1ab_18571

| | | |
|---|---|---|
| Antisense | 5′ UCAAAUACGACUCUGUCUUUU 3′ | 2581 |
| Sense | 5′ ACAGAGUCGUAUUUGA 3′ | 2582 |

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
Atwell et al. J. Mol. Biol. 1997, 270: 26-35;
Ausubel et al. (eds.), CURRENT PROTOCOLS IN MOLECULAR BIOLOGY, John Wiley &Sons, NY (1993);
Ausubel, F. M. et al. eds., SHORT PROTOCOLS IN MOLECULAR BIOLOGY (4th Ed. 1999) John Wiley & Sons, NY. (ISBN 0-471-32938-X); CONTROLLED DRUG BIOAVAILABILITY, DRUG PRODUCT DESIGN AND PERFORMANCE, Smolen and Ball (eds.), Wiley, New York (1984);
Giege, R. and Ducruix, A. Barrett, CRYSTALLIZATION OF NUCLEIC ACIDS AND PROTEINS, a Practical Approach, 2nd ea., pp. 20 1-16, Oxford University Press, New York, New York, (1999);
Goodson, in MEDICAL APPLICATIONS OF CONTROLLED RELEASE, vol. 2, pp. 115-138 (1984);
Hammerling, et al., in: MONOCLONAL ANTIBODIES AND T-CELL HYBRIDOMAS 563-681 (Elsevier, N.Y., 1981;
Harlow et al., ANTIBODIES: A LABORATORY MANUAL, (Cold Spring Harbor Laboratory Press, 2nd ed. 1988);
Kabat et al., SEQUENCES OF PROTEINS OF IMMUNOLOGICAL INTEREST (National Institutes of Health, Bethesda, Md. (1987) and (1991);
Kabat, E. A., et al. (1991) SEQUENCES OF PROTEINS OF IMMUNOLOGICAL INTEREST, Fifth Edition, U.S. Department of Health and Human Services, NIH Publication No. 91-3242;
Kontermann and Dubel eds., ANTIBODY ENGINEERING (2001) Springer-Verlag. New York. 790 pp. (ISBN 3-540-41354-5).

Kriegler, Gene Transfer and Expression, A Laboratory Manual, Stockton Press, NY (1990);
Lu and Weiner eds., CLONING AND EXPRESSION VECTORS FOR GENE FUNCTION ANALYSIS (2001) BioTechniques Press. Westborough, MA. 298 pp. (ISBN 1-881299-21-X).
MEDICAL APPLICATIONS OF CONTROLLED RELEASE, Langer and Wise (eds.), CRC Pres., Boca Raton, Fla. (1974);
Old, R. W. & S. B. Primrose, PRINCIPLES OF GENE MANIPULATION: AN INTRODUCTION TO GENETIC ENGINEERING (3d Ed. 1985) Blackwell Scientific Publications, Boston. Studies in Microbiology; V.2:409 pp. (ISBN 0-632-01318-4).
Sambrook, J. et al. eds., MOLECULAR CLONING: A LABORATORY MANUAL (2d Ed. 1989) Cold Spring Harbor Laboratory Press, NY. Vols. 1-3. (ISBN 0-87969-309-6).
SUSTAINED AND CONTROLLED RELEASE DRUG DELIVERY SYSTEMS, J. R. Robinson, ed., Marcel Dekker, Inc., New York, 1978
Winnacker, E. L. FROM GENES TO CLONES: INTRODUCTION TO GENE TECHNOLOGY (1987) VCH Publishers, NY (translated by Horst Ibelgaufts). 634 pp. (ISBN 0-89573-614-4).

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1-109. (canceled)

110. A method of treating a disease or a disorder in a subject, comprising administering to the subject a therapeutically effective amount of a branched oligonucleotide compound comprising two or more oligonucleotides covalently bound to one another, wherein the branched oligonucleotide compound is delivered to a lung tissue in the subject.

111. The method of claim 110, wherein the branched oligonucleotide compound is administered by intratracheal (IT) injection, nasal delivery, or a combination thereof.

112. The method of claim 110, wherein the branched oligonucleotide compound accumulates in the lung tissue to a greater extent than a non-branched oligonucleotide compound when administered by intratracheal (IT) injection.

113-205. (canceled)

206. The method of claim 111, wherein the intratracheal injection comprises intratracheal inhalation or intratracheal instillation.

207. (canceled)

208. The method of claim 110, wherein the branched oligonucleotide compound is administered to the subject with a nebulizer.

209. The method of claim 110, wherein the branched oligonucleotide compound is formulated as a dry powder.

210. The method of claim 110, wherein administering the branched oligonucleotide compound to the subject causes a decrease in a target gene mRNA in one or more of club cells and alveoli cells of the lung tissue.

211. The method of claim 110, wherein the two or more oligonucleotides comprise two or more RNA molecules each comprising 15 to 35 nucleotides in length, and a sequence substantially complementary to a target nucleic acid sequence in the lung tissue, wherein the two or more RNA molecules are connected to one another by one or more moieties independently selected from a linker, a spacer, a branching point, and a combination thereof.

212. (canceled)

213. The method of claim 211, wherein each of the two or more RNA molecules comprises a dsRNA comprising a sense strand and an antisense strand, wherein each antisense strand independently comprises a sequence substantially complementary to the target nucleic acid sequence.

214-222. (canceled)

223. The method of claim 213, wherein the dsRNA comprises at least one single stranded nucleotide overhang.

224-225. (canceled)

226. The method of claim 213, wherein the dsRNA comprises at least one modified nucleotide.

227. The method of claim 226, wherein the at least modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, or a non-natural base comprising nucleotide.

228. The method of claim 213, wherein the dsRNA comprises at least one modified internucleotide linkage, wherein the at least one modified internucleotide linkage comprises a phosphorothioate internucleotide linkage.

229-243. (canceled)

244. The method of claim 213, wherein the antisense strand comprises a 5′ vinyl phosphonate.

245. The method of claim 213, wherein a functional moiety is linked to one or both of the 5′ end and the 3′ end of the sense strand.

246. (canceled)

247. The method of claim 245, wherein the functional moiety comprises a hydrophobic moiety.

248. The method of claim 247, wherein the hydrophobic moiety is selected from the group consisting of fatty acids, steroids, secosteroids, lipids, gangliosides, nucleoside analogs, endocannabinoids, vitamins, and a mixture thereof.

249. (canceled)

250. The method of claim 248, wherein the fatty acid is selected from the group consisting of eicosapentaenoic acid (EPA), docosahexaenoic acid (DHA), and docosanoic acid (DCA).

251-252. (canceled)

253. The method of claim 245, wherein the functional moiety is linked to one or both of the antisense strand and sense strand by a linker.

254-261. (canceled)

262. A method of delivering a branched oligonucleotide compound to a lung tissue of a subject, comprising administering the branched oligonucleotide compound to the subject via intratracheal (IT) injection, nasal delivery, or a combination thereof, wherein the branched oligonucleotide compound comprises two or more oligonucleotides covalently bound to one another.

263-268. (canceled)
